(19) United States
(12) Patent Application Publication
(10) Pub. No.: US 2026/0327221 A1
(43) Pub. Date: Oct. 22, 2026

(54) INHIBITORS OF CYCLIC GMP-AMP SYNTHASE AND USES THEREOF

(71) Applicant: Ventus Therapeutics U.S., Inc., Waltham, MA (US)

(72) Inventors: Ramsay Beveridge, Pointe-Claire (CA); Stephane Ciblat, Montreal (CA); Patrick Cyr, Saint-Constant (CA)

(73) Assignee: Ventus Therapeutics U.S., Inc., Waltham, MA (US)

(21) Appl. No.: 19/482,373

(22) PCT Filed: May 9, 2024
(86) PCT No.: PCT/US2024/028639

§ 371 Date: Nov. 7, 2025

Related U.S. Application Data
(60) Provisional application No. 63/501,381, filed on May 10, 2023.

(51) Int. Cl. C07D 417/14; A61K 31/433; A61K 31/4439; A61K 31/454; A61K 31/5377; A61K 31/541; A61K 31/547; A61K 31/554; A61K 31/675; C07D 495/08; C07D 495/10; C07F 9/6558

(52) U.S. Cl. CPC C07D 417/14; A61K 31/433; A61K 31/4439; A61K 31/454; A61K 31/5377; A61K 31/541; A61K 31/547; A61K 31/554; A61K 31/675; C07D 495/08; C07D 495/10; C07F 9/65586

(57) ABSTRACT

The present disclosure relates to compounds of Formula (I): or a pharmaceutically acceptable salt thereof, wherein Ring A, R¹, Ring C, R^{3A}, R⁴, m, and n are described herein, methods of preparation, methods of treatment, and pharmaceutical compositions comprising same. The present disclosure further relates to the use of the compounds of Formula (I), and pharmaceutically acceptable salts thereof, in the treatment of cGAS-related diseases and disorders.

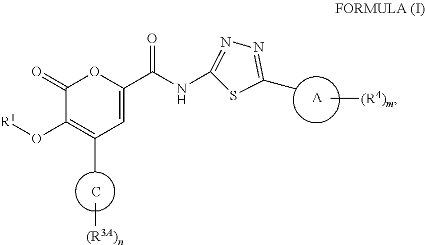

### RELATED APPLICATIONS

This application claims priority under 35 U.S.C. § 119(e) to U.S. provisional patent application, U.S. Ser. No. 63/501,381, filed May 10, 2023, the entire contents of which is incorporated herein by reference.

### BACKGROUND

Aberrant accumulation of cytosolic DNA induces type I interferons and other cytokines that are important for antimicrobial defense but can also induce autoimmunity. This DNA signaling pathway requires the stimulator of interferon genes (STING) adapter protein and the transcription factors NF-κB and IRF3, but the mechanism of DNA sensing was unclear until recently. It is now understood that mammalian cytosolic extracts synthesize cyclic GMP-AMP (cGAMP) in vitro from ATP and GTP in the presence of DNA rather than RNA (WO 2014/099824). DNA transfection or DNA virus infection of mammalian cells also trigger the production of cGAMP. cGAMP binds to STING, leading to IRF3 activation and induction of interferon-β (IFNβ). Thus, cGAMP is the first cyclic dinucleotide in metazoans, and cGAMP functions as an endogenous secondary messenger that induces interferon production in response to cytosolic DNA.

cGAMP synthase (cGAS) is an enzyme that intervenes in the synthesis of cyclic GMP-AMP and belongs to the nucleotidyltransferase family. Overexpression of cGAS activates the transcription factor IRF3 and induces IFNβ in a STING-dependent manner. Knockdown of cGAS inhibits IRF3 activation and IFNβ induction by DNA transfection or DNA virus infection, cGAS binds to DNA in the cytoplasm and catalyzes cGAMP synthesis. These findings indicate that cGAS is a cytosolic DNA sensor that induces interferons by producing the second messenger cGAMP.

The critical role of cGAS in cytosolic DNA sensing has been established in different pathogenic bacteria, viruses, and retroviruses (US 2021/0155625). Additionally, cGAS is essential in various other biological processes, such as cellular senescence and recognition of ruptured micronuclei in the surveillance of potential cancer cells.

There is a need for therapeutic agents that target cGAS. Small molecule inhibitors that are specific for cGAS would be of great value in treating diseases that arise from inappropriate cGAS activity and the resulting undesired type I interferon activity. This present disclosure is intended to fill this unmet need associated with current cGAS inhibition therapy.

### SUMMARY

Provided herein are cGAS inhibitors of Formula (I):

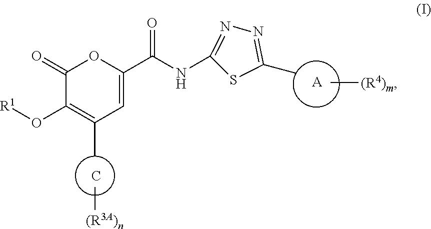

and pharmaceutically acceptable salts thereof, wherein Ring A, Ring C, R¹, R^{3A}, R⁴, m, and n are as described herein.

Further provided are methods of preparation, methods of treatment, and pharmaceutical compositions comprising same.

### Definitions

Definitions of specific functional groups and chemical terms are described in more detail below. The chemical elements are identified in accordance with the Periodic Table of the Elements, CAS version, Handbook of Chemistry and Physics. 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Laock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Compounds described herein can additionally encompasses individual isomers substantially free of other isomers, and alternatively, as mixtures of various isomers.

Unless otherwise stated, compounds described herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₂), n-octyl (C₅) and the like. Unless otherwise specified, each instance of an alkyl group is independently unsubstituted (an “unsubstituted alkyl”) or substituted (a “substituted alkyl”) with one or more substituents. In some embodiments, the alkyl group is an unsubstituted C₁₋₁₀ alkyl (e.g., —CH₃). In some embodiments, the alkyl group is a substituted C₁₋₁₀ alkyl.

“Haloalkyl” refers to a substituted alkyl group, as defined herein, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 10 carbon atoms and one or more carbon-carbon double bonds (e.g., 1, 2, 3, or 4 double bonds) (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), I-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In some embodiments, the alkenyl group is an unsubstituted C₂₋₁₀ alkenyl. In some embodiments, the alkenyl group is a substituted C₂₋₁₀ alkenyl.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In some embodiments, the alkynyl group is an unsubstituted C₂₋₁₀ alkynyl. In some embodiments, the alkynyl group is a substituted C₂₋₁₀alkynyl.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 9 ring carbon atoms (“C₃₋₉ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C⁵⁻¹⁰ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in some embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons designate the number of carbons in the polycyclic ring system. Unless otherwise specified, each instance of a carbocyclyl group is independently unsubstituted (an “unsubstituted carbocyclyl”) or substituted (a “substituted carbocyclyl”) with one or more substituents. In some embodiments, the carbocyclyl group is an unsubstituted C₃₋₁₄ carbocyclyl. In some embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In some embodiments, the cycloalkyl group is an unsubstituted C₃₋₁₄ cycloalkyl. In some embodiments, the cycloalkyl group is a substituted C₃₋₁₄ cycloalkyl.

“Heterocyclyl” or “heterocyclic” refers to a radical of a 3- to 14-membered non-aromatic ring system having ring carbon atoms and 1 to 4 ring heteroatoms, wherein each ring heteroatom is independently selected from nitrogen, oxygen, and sulfur (“3-14 membered heterocyclyl”). It is understood that the ring sulfur or ring nitrogen may exist in an oxygenated state, such as an N-oxide (N—O), sulfonyl (S(═O)₂) or sulfinyl (S═O) ring heteroatom. In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes (i) polycyclic ring systems wherein the heterocyclyl ring, as defined above, is fused (e.g., spiro-fused or ring fused) or bridged with one or more carbocyclyl groups wherein the point of attachment is either on the carbocyclyl or heterocyclyl ring, or (ii) polycyclic ring systems wherein the heterocyclyl ring, as defined above, is fused with one or more aryl or heteroaryl groups, wherein the point of attachment is on the heterocyclyl ring, and in such instances (i) and (ii), the number of ring members designate the number of ring members in the polycyclic ring system. Unless otherwise specified, each instance of heterocyclyl is independently unsubstituted (an “unsubstituted heterocyclyl”) or substituted (a “substituted heterocyclyl”) with one or more substituents. In some embodiments, the heterocyclyl group is an unsubstituted 3-14 membered heterocyclyl. In some embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetra-hydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydro-furo[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms designate the number of carbon atoms in the polycyclic ring system. Unless otherwise specified, each instance of an aryl group is independently unsubstituted (an “unsubstituted aryl”) or substituted (a “substituted aryl”) with one or more substituents. In some embodiments, the aryl group is an unsubstituted C₆₋₁₄ aryl. In some embodiments, the aryl group is a substituted C₆₋₁₄ aryl.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” also includes polycyclic ring systems wherein the heteroaryl ring, as defined above, (i) is fused with one or more carbocyclyl or heterocyclyl groups wherein the point of attachment is on the heteroaryl ring, or (ii) is fused with one or more aryl groups wherein the point of attachment is either on the aryl or heteroaryl ring, and in such instances (i) and (ii), the number of ring members designate the number of ring members in the fused polycyclic ring system. Polycyclic heteroaryl groups wherein one ring does not contain a ring heteroatom (e.g., indolyl, quinolinyl, carbazolyl, and the like), the point of attachment can be on either ring, i.e., either the ring bearing a ring heteroatom (e.g., 2-indolyl) or the ring that does not contain a ring heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In some embodiments, the heteroaryl group is an unsubstituted 5-14 membered heteroaryl. In some embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Ortho” position refers to the 2-position on an aryl or heteroaryl ring, relative to the 1-position which is the point of attachment.

“Halo” or “halogen” refers to fluorine (fluoro, —F), chlorine (chloro, —Cl), bromine (bromo, —Br), or iodine (iodo, —I) radicals.

“Saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, haloalkylene is the divalent moiety of haloalkyl alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl. By way of example, alkylene may be a C₁₋₆ (alkylene, which may be linear or branched. An alkylene may further be a C₁₋₄ alkylene. Exemplary C₁₋₄ alkylene groups include, but are not limited to, —CH₂—, —CH(CH₃)—, —C(CH₃)₂—, —CH₂CH₂—, —CH₂CH(CH₃)—, —CH₂C(CH₃)₂—. —CH₂CH₂CH₂—. —CH₂CH₂CH₂CH₂—, and the like.

“Salt” refers to any and all salts, including pharmaceutically acceptable salts.

“Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Examples of pharmaceutically acceptable acid addition salts include, but are not limited to, salts formed from inorganic acids, such as hydrochloric acid, hydrobromic acid, phosphoric acid, sulfuric acid and perchloric acid salts, or salts formed from organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “free base” refers to a neutral non-ionized form of a compound which is not a salt or pharmaceutically acceptable salt.

A “leaving group” is an art-understood term referring to a molecular fragment that departs with a pair of electrons in heterolytic bond cleavage, wherein the molecular fragment is an anion or neutral molecule. See, for example, Smith, March Advanced Organic Chemistry 6th ed. (501-502). Exemplary leaving groups include, but are not limited to, halo (e.g., chloro, bromo, iodo) and sulfonyl substituted hydroxyl groups (e.g., —O-tosyl, —O-mesyl, and —O-besyl).

A “patient” or “subject” is used interchangeably herein, and refers to a mammal, e.g., a human, mouse, rat, guinea pig, dog, cat, horse, cow, pig, or non-human primate, such as a monkey, chimpanzee, baboon, or rhesus. In certain embodiments, the patient or subject is a human.

“Effective amount” or “therapeutically effective amount” are used interchangeably herein, and refer to an amount of the compound sufficient to provide a therapeutic benefit in the treatment of a disease, disorder or condition, or to delay or minimize one or more symptoms associated with the disease, disorder or condition in a subject in need thereof. An effective amount can encompass an amount that improves overall therapy, reduces or avoids symptoms or causes of disease or condition, or enhances the therapeutic efficacy of another therapeutic agent. The effective amount of a compound may vary depending on such factors as the desired biological endpoint, the pharmacokinetics of the compound, the disease being treated, the mode of administration, and the age, health, and condition of the subject.

“Disease”, “disorder”, “condition”, or “state” are used interchangeably herein.

“Treating” or “treat” or “treatment” describes the management and care of a subject in need thereof, for the purpose of combating a disease, condition, or disorder in the subject, and includes the administration of a compound, or a pharmaceutically acceptable salt thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition, and therefore includes: (1) delaying the appearance of clinical symptoms of the state, disorder or condition developing in a subject that may be afflicted with or predisposed to the state, disorder or condition but does not yet experience or display clinical or subclinical symptoms of the state, disorder or condition, (2) arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Modulate”, “modulating” and the like, refer to the ability of a compound to change the activity of a particular biological process (e.g., cGAS activity) in a cell relative to a control.

“Inhibition”, “inhibiting”, “inhibit” and “inhibitor”, and the like, refer to the ability of a compound to reduce, slow, halt or prevent activity of a particular biological process (e.g., cGAS activity) in a cell relative to a control.

The phrase “at least one” refers to one instance or more than one instance.

The articles “a” and “an” are used in this disclosure to refer to one or more than one (i.e., to at least one) of the grammatical object of the article.

The term “and/or” is used in this disclosure to mean either “and” or “or” unless indicated otherwise.

### DETAILED DESCRIPTION

### i. Compounds

Provided herein are compounds of Formula (I):

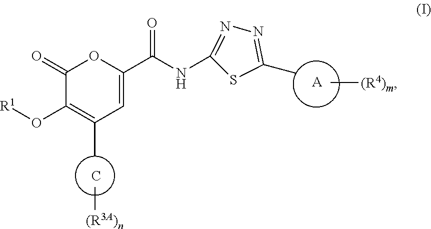

and pharmaceutically acceptable salts thereof, wherein:
   Ring A is a 5-membered monocyclic heteroaryl;
   R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄ alkyl, or C₁-C₄haloalkyl, wherein the alkyl and haloalkyl are independently substituted with 0, 1, 2, 3, or 4 R^{1D}; each R^{1C} is independently hydrogen, C₁-C₃ alkyl, C₁-C₃ haloalkyl, or —OR^{1F}; each R^{1D} is independently halogen, —OR^{1F}, or —N(R^{1F})₂; each R^{1F} is independently hydrogen, C₁-C₃ alkyl, or C₁-C₃ haloalkyl;
   L₃ is a bond, C₁-C₃ alkylene, or —(C₁-C₃ alkylene)-O—, wherein the alkylene is independently substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O;
   Ring C is a C₆-C₁₀ aryl or 5- to 10-membered heteroaryl;
   each R^{3A} is independently C₁-C₃ alkyl, C₂-C₃ alkenyl, C₂-C₃ alkynyl, halogen, -L₁-CN, -L₁-SOR^{3C}, -L₁-SO₂R^{3C}, -L₁-SR^{3B}, -L₁-PO(R^{3C})₂, -L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
   each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
   each R^{3C} is independently C₁-C₃ alkyl or C₁-C₃ haloalkyl;
   each R^{3D} is independently halogen, —OR^{3E}, —CN, C₁-C₃ alkyl, or C₁-C₃ haloalkyl;
   each R^{3E} is independently hydrogen, C₁-C₄ alkyl, or C₁-C₄ haloalkyl;
   each R⁴ is independently halogen, —CN, -L₂-OR^{4A}, -L₂-N(R^{4B})₂, C₁-C₆ alkyl, or C₁-C₆ haloalkyl;
   each R^{4A} and R^{4B} are independently hydrogen. C₁-C₃ alkyl, C₁-C₃ haloalkyl, or —C(═O)R^{4C}, wherein R^{4C} is C₁-C₆ alkyl or C₁-C₆ haloalkyl;
   each L₁ and L₂ is independently a bond, C₁-C₃ alkylene, or C₁-C₃ haloalkylene;
   m is 0, 1, or 2; and
   n is 0, 1, 2, 3, or 4.

In some embodiments, the compound is a compound of Formula (I), or a pharmaceutically acceptable salt thereof, wherein:
R¹ is C₁-C₆ alkyl substituted with 0, 1, 2, 3, or 4 R^{1A}; each R^{1A} is independently —OR^{1B}; each R^{1B} is independently hydrogen or C₁-C₄ alkyl substituted with 0, 1, 2, 3, or 4 R^{1D}; each R^{1D} is independently halogen or —OR^{1F}; and each R^{1F} is independently hydrogen or C₁-C₃ alkyl;
-L₃-(C₃-C₆ carbocyclyl), wherein the carbocyclyl is substituted with 0, 1, 2, 3, or 4 R^{1A}; each R^{1A} is independently —OR^{1B}; each R^{1B} is independently hydrogen or C₁-C₄ alkyl substituted with 0, 1, 2, 3, or 4 R^{1D}; each R^{1D} is independently halogen or —OR^{1F}; each R^{1F} is independently hydrogen or C₁-C₃ alkyl; and L₃ is a bond or —(C₁-C₃ alkylene)-O—; or
-L₃-(4- to 10-membered heterocyclyl), wherein the heterocyclyl is substituted with 0, 1, 2, 3, or 4 R^{1A}; each R^{1A} is independently —OR^{1B}, —(C₁-C₃ alkylene)-OR^{1B}, or —(C₁-C₃ alkylene)-SR^{1B}, or two instances of R^{1A} are taken together to form ═O; each R^{1B} is independently hydrogen or C₁-C₄ alkyl substituted with 0, 1, 2, 3, or 4 R^{1D}; each R^{1D} is independently halogen or —OR^{1F}; each R^{1F} is independently hydrogen or C₁-C₃ alkyl; L₃ is a bond, C₁-C₃ alkylene, or —(C₁-C₃ alkylene)-O—, wherein the alkylene is substituted with 0, 1, 2, 3, or 4 R^{1E}; and each R^{1E} is independently —(C₁-C₃ alkylene)-OR^{1B}, —OR^{1B}, or two instances of R^{1E} are taken together to form ═O;
each R^{3A} is independently C₁-C₃ alkyl, halogen. -L₁-CN, or -L₁-OR^{3B}, wherein the alkyl is independently substituted with 0, 1, 2, 3, or 4 R^{3D};
each R^{3B} is independently hydrogen or C₁-C₃ alkyl substituted with 0, 1, 2, 3, or 4 R^{3D};
each R^{3C} is independently C₁-C₃ alkyl;
each R^{3D} is independently halogen or —OR^{3E};
R^{3E} is C₁-C₃ alkyl;
each R⁴ is independently halogen, -L₂-N(R^{4B})₂, C₁-C₆ alkyl, or C₁-C₆ haloalkyl; and each R^{4B} is independently hydrogen, C₁-C₃ alkyl, or C₁-C₃ haloalkyl;
each L₁ and L₂ is independently a bond or C₁-C₃ alkylene;
m is 1 or 2; and
n is 1 or 2.

In some embodiments, the compound is a compound of Formula (I), or a pharmaceutically acceptable salt thereof, wherein:
R¹ is C₁-C₆ alkyl substituted with 1 or 2 R^{1A}; each R^{1A} is independently —OR^{1B}; and each R^{1B} is independently hydrogen or C₁-C₄ alkyl substituted with 0 R^{1D};
-L₃-(C₃-C₆ carbocyclyl), wherein the carbocyclyl is substituted with 1 R^{1A}; R^{1A} is —OR^{1B}; R^{1B} is hydrogen, and L₃ is bond; or
-L₃-(4- to 10-membered heterocyclyl), wherein the heterocyclyl is substituted with 0 R^{1A}; and L₃ is C₁-C₃ alkylene substituted with 0 R^{1E};
each R^{3A} is independently C₁-C₃ alkyl, halogen, -L₁-CN, or -L₁-OR^{3B}, wherein the alkyl is independently substituted with 0, 1, 2, 3, or 4 R^{3D};
each R^{3B} is independently hydrogen or C₁-C₃ alkyl substituted with 0, 1, 2, 3, or 4 R^{3D};
each R^{3C} is independently C₁-C₃ alkyl;
each R^{1D} is independently halogen or —OR^{3E}
R^{3E} is C₁-C₃ alkyl;
each R⁴ is independently -L₂-NH₂ or C₁-C₆ alkyl;
each L₁ and L₂ is independently a bond or C₁-C₃ alkylene;
m is 1 or 2; and
n is 1 or 2.

In some additional embodiments of Formula (I), the compound is of Formula (I′):

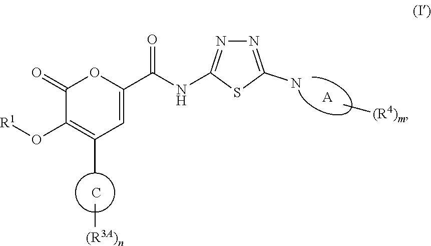

or a pharmaceutically acceptable salt thereof, wherein a nitrogen atom of the heteroaryl Ring A is directly linked to the thiadiazole moiety.

Applicants have found that compounds of Formula (I), comprising the combination of an —OR¹ group at the C₃ position of the pyrone ring, an aryl or heteroaryl moiety at the C₄ position of the pyrone ring, and a 5-membered monocyclic heteroaryl Ring A, show improvement in one or more desirable drug-like properties, such as improvement in unbound clearance, permeability, bioavailability, hcGAS potency, and/or solubility. Applicants have additionally found incorporating at least one -L¹-OR^{3B} group, -L₁-CN group, or halogen group, which are exemplary substituents of group R^{3A}, particularly at one or both ortho positions to the point of attachment of Ring C to the pyrone ring, may show additional improvements in one or more of these desirable properties. As a non-limiting example, as shown in Table D of the Examples, inclusion of an additional ortho -L¹-OR^{3B} group (e.g., —OCH₃) or halogen group to Compound 1 (“A2” activity) provides Compound 44 (“A1” activity) and Compound 41 (“A1” activity) with improved cGAS potency. Further non-limiting examples include Compound 24A*, comprising an ortho —CN and ortho C₁-C₃ alkyl group, and Compound 45, comprising an ortho —CN and ortho halogen group, each with “A1” activity.

For example, in certain embodiments, the compound of Formula (I) is of Formula (I″):

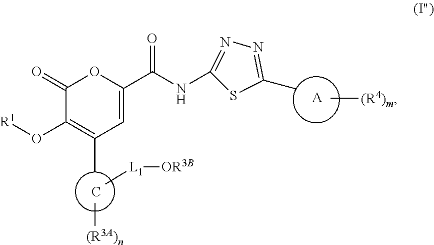

and pharmaceutically acceptable salts thereof, wherein n is 0, 1, 2, or 3. In certain embodiments, -L₁-OR^{3B} is ortho to the point of attachment to the pyrone ring. In certain embodiments, Ring C further comprises a second R^{3A} group ortho to the point of attachment selected from halogen, -L₁-OR^{3B}, or -L₁-CN, and optionally comprises additional R^{3A} groups, wherein n is 0, 1, or 2.

In other embodiments, the compound of Formula (I) is of Formula (I′″):

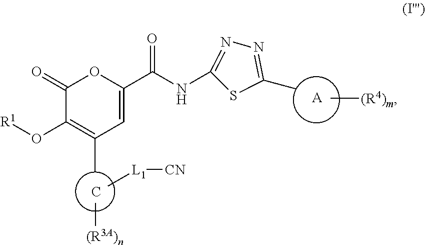

and pharmaceutically acceptable salts thereof, wherein n is 0, 1, 2, or 3. In certain embodiments, -L₁-CN is ortho to the point of attachment to the pyrone ring. In certain embodiments, Ring C further comprises a second R^{3A} group ortho to the point of attachment selected from halogen, -L₁-OR^{3B}, or -L₁-CN, and optionally comprises additional R^{3A} groups, wherein n is 0, 1, or 2.

Additional embodiments are further described below and herein.

(a) R¹, R^{1A}, R^{1B}, R^{1C}, R^{1D}, R^{1E}, R^{1F}, L₃, Ring C, n, R^{3A}, R^{3B}, R^{3C}, R^{3D}, R^{3E}, and L₁

As generally described herein, R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄ alkyl, or C₁-C₄haloalkyl, wherein the alkyl and haloalkyl are independently substituted with 0, 1, 2, 3, or 4 R^{1D}; each R^{1C} is independently hydrogen, C₁-C₃ alkyl, C₁-C₃haloalkyl, or —OR^{1F}; each R^{1D} is independently halogen, —OR^{1F}, or —N(R^{1F})₂; each R^{1F} is independently hydrogen, C₁-C₃ alkyl, or C₁-C₃ haloalkyl; L₃ is a bond, C₁-C₃ alkylene, or —(C₁-C₃ alkylene)-O—, wherein the alkylene is independently substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments, R¹ is C₁-C₆ alkyl substituted with 0, 1, 2, 3, or 4 R^{1A}. In some embodiments, R¹ is C₁-C₄ alkyl substituted with 0, 1, 2, 3, or 4 R^{1A}. In some embodiments, R¹ is C₁-C₃ alkyl substituted with 0, 1, 2, 3, or 4 R^{1A}. In some embodiments, R¹ is C₁-C₂ alkyl substituted with 0, 1, 2, 3, or 4 R^{1A}.

In some embodiments. R¹ is C₂-C₆ alkenyl substituted with 0, 1, 2, 3, or 4 R^{1A}. In some embodiments, R¹ is C₂-C₄ alkenyl substituted with 0, 1, 2, 3, or 4 R^{1A}. In some embodiments, R¹ is C₂-C₃ alkenyl substituted with 0, 1, 2, 3, or 4 R^{1A}.

In some embodiments, R¹ is C₂-C₆ alkynyl substituted with 0, 1, 2, 3, or 4 R^{1A}. In some embodiments, R¹ is C₂-C₄ alkynyl substituted with 0, 1, 2, 3, or 4 R^{1A}. In some embodiments. R¹ is C₂-C₃ alkynyl substituted with 0, 1, 2, or 3 R^{1A}.

In some embodiments. R¹ is -L₃-(C₃-C₆ carbocyclyl), wherein the carbocyclyl is substituted with 0, 1, 2, 3, or 4 R^{1A}. In some embodiments. R¹ is -L₃-(C₃-C₄ carbocyclyl), wherein the carbocyclyl is substituted with 0, 1, 2, 3, or 4 R^{1A}. In some embodiments, R¹ is -L₃-(C₅-C₆ carbocyclyl), wherein the carbocyclyl is substituted with 0, 1, 2, 3, or 4 R^{1A}.

In some embodiments, R¹ is -L₃-(4- to 10-membered heterocyclyl), wherein the heterocyclyl is substituted with 0, 1, 2, 3, or 4 R^{1A}. In some embodiments. R¹ is -L₃-(7- to 10-membered heterocyclyl), wherein the heterocyclyl is substituted with 0, 1, 2, 3, or 4 R^{1A}. In some embodiments. R¹ is -L-(4- to 6-membered heterocyclyl), wherein the heterocyclyl is substituted with 0, 1, 2, 3, or 4 R^{1A}. In some embodiments, R¹ is -L₃-(5- to 6-membered heterocyclyl), wherein the heterocyclyl is substituted with 0, 1, 2, 3, or 4 R^{1A}.

In some embodiments. R¹ is C₁-C₆ alkyl substituted with 0 R^{1A}.

In some embodiments, each R^{1B} is independently hydrogen. C₁-C₃ alkyl, or C₁-C₃ haloalkyl substituted with 0, 1, 2, 3, or 4 R^{1D}.

In some embodiments, R¹ is C₁-C₆ alkyl substituted with 1 R^{1A}; R^{1A} is halogen, —OR^{1B}, —N(R^{1B})₂, —(C₁-C₃ alkylene)-OR^{1B}, or —(C₁-C₃ alkylene)-SR^{1B}; each R^{1B} is independently hydrogen, C₁-C₃ alkyl, or C₁-C₃ haloalkyl substituted with 0, 1, 2, 3, or 4 R^{1D}; each R^{1D} is independently halogen, —OR^{1F}, or —N(R^{1F})₂; and each R^{1F} is independently hydrogen, C₁-C₃ alkyl, or C₁-C₃ haloalkyl.

In some embodiments. R¹ is C₁-C₆ alkyl substituted with 1 R^{1A}; R^{1A} is —OR^{1B}; and R^{1B} is hydrogen. C₁-C₃ alkyl, or C₁-C₃ haloalkyl substituted with 0 R^{1D}.

In some embodiments, R¹ is C₁-C₆ alkyl substituted with 1 R^{1A}; R^{1A} is —OR^{1B}; R^{1B} is C₁-C₃ alkyl or C₁-C₃ haloalkyl substituted with 1 R^{1D}; R^{1D} is —OR^{1F}; and R^{1F} is hydrogen, C₁-C₃ alkyl, or C₁-C₃ haloalkyl.

In some embodiments, R¹ is C₁-C₆ alkyl substituted with 1 R^{1A}; R^{1A} is —OR^{1B}; R^{1B} is C₁-C₃ alkyl or C₁-C₃ haloalkyl substituted with 1 R^{1D}; R^{1D} is —OR^{1F}; and R^{1F} is hydrogen.

In some embodiments. R¹ is C₁-C₆ alkyl substituted with 1 R^{1A}; R^{1A} is —OR^{1B}; R^{1B} is C₁-C₃ alkyl or C₁-C₃ haloalkyl substituted with 1 R^{1D}; R^{1D} is —OR^{1F}; and R^{1F} is C₁-C₃ alkyl.

In some embodiments. R¹ is C₁-C₆ alkyl substituted with 1 R^{1A}; R^{1A} is —OR^{1B}; and R^{1B} is hydrogen or C₁-C₃ alkyl substituted with 0 R^{1D}.

In some embodiments, R¹ is C₁-C₆ alkyl substituted with 1 R^{1A}; R^{1A} is —OR^{1B}; and R^{1B} is hydrogen.

In some embodiments, R¹ is C₁-C₆ alkyl substituted with 1 R^{1D}; R^{1A} is —OR^{1B}; and R^{1B} is C₁-C₃ alkyl substituted with 0 R^{1D}.

In some embodiments, R¹ is C₁-C₆ alkyl substituted with 1 R^{1A}; R^{1A} is —OR^{1B}; R^{1B} is C₁-C₃ alkyl substituted with 1 R^{1D}; R^{1D} is —OR^{1F}; and R^{1F} is hydrogen, C₁-C₃ alkyl, or C₁-C₃ haloalkyl.

In some embodiments, R¹ is C₁-C₆ alkyl substituted with 1 R^{1A}; R^{1A} is —OR^{1B}; R^{1B} is C₁-C₃ alkyl substituted with 1 R^{1D}; R^{1D} is —OR^{1F}; and R^{1F} is hydrogen.

In some embodiments. R¹ is C₁-C₆ alkyl substituted with 1 R^{1A}; R^{1A} is —OR^{1A}; R^{1B} is C₁-C₃ alkyl substituted with 1 R^{1D}; R^{1D} is —OR^{1F}; and R^{1F} is C₁-C₃alkyl.

In some embodiments. R¹ is C₁-C₆ alkyl substituted with 2 R^{1A}; each R^{1A} is independently —OR^{1B}; each R^{1B} is independently hydrogen, C₁-C₃ alkyl, or C₁-C₃ haloalkyl independently substituted with 0, 1, 2, 3, or 4 R^{1D} each R^{1D} is independently halogen, —OR^{1F}, or —N(R^{1F})₂; and each R^{1F} is independently hydrogen, C₁-C₃ alkyl, or C₁-C₃ haloalkyl.

In some embodiments, R¹ is C₁-C₆ alkyl substituted with 2 R^{1A}; each R^{1A} is independently —OR^{1B}; and each R^{1B} is independently hydrogen or C₁-C₃ alkyl substituted with 0 R^{1D}.

In some embodiments. R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}.

In some embodiments. R¹ is C₁-C₆ alkyl substituted with 2 R^{1A}; and two instances of R^{1A} are taken together to form ═O.

In some embodiments, R¹ is -L₃-(C₃-C₆ carbocyclyl), wherein the carbocyclyl is substituted with 0 R^{1A}; L₃ is a bond, C₁-C₃ alkylene, or —(C₁-C₃ alkylene)-O—, wherein the alkylene is substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments, R¹ is -L₃-(C₃-C₆ carbocyclyl), wherein the carbocyclyl is substituted with 1 R^{1A}; R^{1A} is halogen, —OR^{1B}, —N(R^{1B})₂, —(C₁-C₃ alkylene)-OR^{1B}, or —(C₁-C₃ alkylene)-SR^{1B}; R^{1B} is hydrogen, C₁-C₃ alkyl, or C₁-C₃ haloalkyl substituted with 0, 1, 2, 3, or 4 R^{1D}; each R^{1D} is independently halogen, —OR^{1F}, or —N(R^{1F})₂; each R^{1F} is independently hydrogen. C₁-C₃ alkyl, or C₁-C₃ haloalkyl; L₃ is a bond, C₁-C₃ alkylene, or —(C₁-C₃ alkylene)-O—, wherein the alkylene is substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments, R¹ is -L₃-(C₃-C₆ carbocyclyl), wherein the carbocyclyl is substituted with 1 R^{1A}; R^{1A} is halogen; L₃ is a bond, C₁-C₃ alkylene, or —(C₁-C₃ alkylene)-O—, wherein the alkylene is substituted with 0, 1, 2, 3, or 4 R^{1E}; and each R^{1E} is independently-(C₁-C₃ alkylene)-OR^{1B} or —OR^{1B}, or two instances of R^{1E} are taken together to form ═O.

In some embodiments. R¹ is -L₃-(C₃-C₆ carbocyclyl), wherein the carbocyclyl is substituted with 1 R^{1A}; R^{1A} is halogen; and L₃ is bond.

In some embodiments, R¹ is -L₃-(C₁-C₆ carbocyclyl), wherein the carbocyclyl is substituted with 1 R^{1A}; R^{1A} is —OR^{1B}; R^{1B} is hydrogen; and L₃ is bond or —(C₁-C₃ alkylene)-O—, wherein the alkylene is substituted with 0, 1, 2, 3, or 4 R^{1E}; and each R^{1E} is independently-(C₁-C₃ alkylene)-OR^{1B} or —OR^{1B}, or two instances of R^{1E} are taken together to form ═O.

In some embodiments, R¹ is -L₃-(C₃-C₆ carbocyclyl), wherein the carbocyclyl is substituted with 1 R^{1A}; R^{1A} is —OR^{1B}; R^{1B} is C₁-C₃ alkyl substituted with 0 R^{1D}; and L₃ is bond or —(C₁-C₃ alkylene)-O—, wherein the alkylene is substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments. R¹ is -L₃-(C₃-C₆ carbocyclyl), wherein the carbocyclyl is substituted with 2 R^{1A}; each R^{1A} is halogen, —OR^{1B}, —N(R^{1B})₂, —(C₁-C₃ alkylene)-OR^{1B}, or —(C₁-C₃ alkylene)-SR^{1B}; each R^{1B} is hydrogen, C₁-C₃ alkyl, or C₁-C₃ haloalkyl substituted with 0, 1, 2, 3, or 4 RID; each R^{1D} is independently halogen, —OR^{1F}, or —N(R^{1F})₂; each R^{1F} is independently hydrogen, C₁-C₃alkyl, or C₁-C₃ haloalkyl; L₃ is a bond, C₁-C₃ alkylene, or —(C₁-C₃ alkylene)-O—, wherein the alkylene is substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments. R¹ is -L₃-(C₃-C₆ carbocyclyl), wherein the carbocyclyl is substituted with 2 R^{1A}; each R^{1A} is —OR^{1B}; each R^{1B} is hydrogen. C₁-C₃ alkyl, or C₁-C₃ haloalkyl substituted with 0 R^{1D}; and L₃ is C₁-C₃ alkylene or —(C₁-C₃ alkylene)-O—, wherein the alkylene is substituted with 0 R^{1E}.

In some embodiments, R¹ is -L₃-(C₃-C₆ carbocyclyl), wherein the carbocyclyl is substituted with 2 R^{1A}; each R^{1A} is —OR^{1B}; each R^{1B} is hydrogen or C₁-C₃ alkyl substituted with 0 R^{1D}; and L₃ is C₁-C₃ alkylene substituted with 0 R^{1E}.

In some embodiments, R¹ is -L₃-(4- to 10-membered heterocyclyl), wherein the heterocyclyl is substituted with 0 R^{1A}; L₃ is a bond, C₁-C₃ alkylene, or —(C₁-C₃ alkylene)-O—, wherein the alkylene is substituted with 0, 1, 2, 3, or 4 R^{1E}; and each R^{1B} is independently —(C₁-C₃ alkylene)-OR^{1B} or —OR^{1B}, or two instances of R^{1E} are taken together to form ═O.

In some embodiments. R¹ is -L₃-(4- to 10-membered heterocyclyl), wherein the heterocyclyl is substituted with 0 R^{1A}; and L₃ is a bond.

In some embodiments, R¹ is -L₃-(4- to 10-membered heterocyclyl), wherein the heterocyclyl is substituted with 0 R^{1A}; L₃ is C₁-C₃ alkylene or —(C₁-C₃ alkylene)-O—, wherein the alkylene is substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments. R¹ is -L₃-(4- to 10-membered heterocyclyl), wherein the heterocyclyl is substituted with 0 R^{1A}; and L₃ is C₁-C₃ alkylene substituted with 0 R^{1E}.

In some embodiments. R¹ is -L₃-(4- to 10-membered heterocyclyl), wherein the heterocyclyl is substituted with 0 R^{1A}; L₃ is C₁-C₃ alkylene substituted with 1 R^{1E}; and R^{1B} is —(C₁-C₃ alkylene)-OR^{1B}, or —OR^{1B}.

In some embodiments, R¹ is -L₃-(4- to 10-membered heterocyclyl), wherein the heterocyclyl is substituted with 0 R^{1A}; L₃ is C₁-C₃ alkylene substituted with 1 R^{1E}; R^{1E} is —(C₁-C₃ alkylene)-OR^{1B}.

In some embodiments, R¹ is -L₃-(4- to 10-membered heterocyclyl), wherein the heterocyclyl is substituted with 0 R^{1A}; L₃ is C₁-C₃ alkylene substituted with 1 R^{1E}; and R^{1E} is —OR^{1B}.

In some embodiments, R¹ is -L₃-(4- to 10-membered heterocyclyl), wherein the heterocyclyl is substituted with 1 R^{1A}; R^{1A} is halogen, —OR^{1B}, —N(R^{1B})₂, —(C₁-C₃ alkylene)-OR^{1B}, or —(C₁-C₃ alkylene)-SR^{1B}; R^{1B} is hydrogen, C₁-C₃ alkyl, or C₁-C₃ haloalkyl substituted with 0, 1, 2, 3, or 4 RID; each R^{1D} is independently halogen, —OR^{1F}, or —N(R^{1F})₂; each R^{1F} is independently hydrogen, C₁-C₃ alkyl, or C₁-C₃ haloalkyl; L₃ is a bond, C₁-C₃ alkylene, or —(C₁-C₃ alkylene)-O—, wherein the alkylene is substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O.

In some embodiments, R¹ is -L₃-(4- to 10-membered heterocyclyl), wherein the heterocyclyl is substituted with 1 R^{1A}; R^{1A} is —(C₁-C₃ alkylene)-SR^{1B}; R^{1B} is hydrogen, C₁-C₃ alkyl, or C₁-C₃ haloalkyl substituted with 0, 1, 2, 3, or 4 R^{1D}; each R^{1D} is independently halogen, —OR^{1F}, or —N(R^{1F})₂; each R^{1F} is independently hydrogen, C₁-C₃ alkyl, or C₁-C₃ haloalkyl; and L₃ is C₁-C₃ alkylene substituted with 0 R^{1E}.

In some embodiments. R¹ is -L₃-(4- to 10-membered heterocyclyl), wherein the heterocyclyl is substituted with 1 R^{1A}; R^{1A} is —(C₁-C₃ alkylene)-SR^{1B}; R^{1B} is C₁-C₃ alkyl substituted with 0 R^{1D}; and L₃ is C₁-C₃ alkylene substituted with 0 R^{1E}.

In some embodiments, R¹ is -L₃-(4- to 10-membered heterocyclyl), wherein the heterocyclyl is substituted with 1 R^{1A}; R^{1A}—(C₁-C₃ alkylene)-OR^{1B}; R^{1B} is hydrogen, C₁-C₃ alkyl, or C₁-C₃ haloalkyl substituted with 0, 1, 2, 3, or 4 R^{1D}; each R^{1D} is independently halogen, —OR^{1F}, or —N(R^{1F})₂; each R^{1F} is independently hydrogen, C₁-C₃ alkyl, or C₁-C₃ haloalkyl; and L₃ is C₁-C₃ alkylene substituted with 0 R^{1E}.

In some embodiments. R¹ is -L₃-(4- to 10-membered heterocyclyl), wherein the heterocyclyl is substituted with 1 R^{1A}; R^{1A} is —(C₁-C₃ alkylene)-OR^{1B}; R^{1B} is hydrogen; and L₃ is C₁-C₃ alkylene substituted with 0 R^{1E}.

In some embodiments, R¹ is -L₃-(4- to 10-membered heterocyclyl), wherein the heterocyclyl is substituted with 1 R^{1A}; R^{1A}—(C₁-C₃ alkylene)-SR^{1B}; R^{1B} is hydrogen, C₁-C₃ alkyl, or C₁-C₃ haloalkyl substituted with 0, 1, 2, 3, or 4 R^{1D}; each R^{1D} is independently halogen, —OR^{1F}, or —N(R^{1F})₂; each R^{1F} is independently hydrogen, C₁-C₃ alkyl, or C₁-C₃ haloalkyl; and L₃ is C₁-C₃ alkylene substituted with 0 R^{1E}.

In some embodiments. R¹ is -L₃-(4- to 10-membered heterocyclyl), wherein the heterocyclyl is substituted with 1 R^{1A}; R^{1A} is —(C₁-C₃ alkylene)-SR^{1B}; R^{1B} is hydrogen; and L₃ is C₁-C₃ alkylene substituted with 0 R^{1E}.

In some embodiments. R¹ is -L₃-(4- to 10-membered heterocyclyl), wherein the heterocyclyl is substituted with 2 R^{1A}; two instances of R^{1A} are taken together to form ═O; and L₃ is C₁-C₃ alkylene substituted with 0 R^{1E}.

In some embodiments, at least one R^{1A} is independently halogen, —OR^{1B}, —N(R^{1B})₂, or —(C₁-C₃ alkylene)-OR^{1B}.

In some embodiments, at least one R^{1A} is independently halogen, —OR^{1B}, or —N(R^{1B})₂.

In some embodiments, at least one R^{1A} is independently halogen.

In some embodiments, at least one R^{1A} is independently —OR^{1B} or —N(R^{1B})₂.

In some embodiments, at least one R^{1A} is independently —OR^{1B} or —(C₁-C₃ alkylene)-OR^{1B}.

In some embodiments, at least one R^{1A} is independently —OR^{1B}.

In some embodiments, at least one R^{1A} is independently —OH. In some embodiments, at least one R^{1A} is independently —O(C₁-C₃ alkyl).

In some embodiments, at least one R^{1A} is independently —N(R^{1B})₂.

In some embodiments, at least one R^{1A} is independently —S(R^{1B}).

In some embodiments, at least one instance of R^{1A} is —C(═O)OR^{1B}.

In some embodiments, at least one instance of R^{1A} is —C(═O)N(R^{1C})₂.

In some embodiments, at least one R^{1C} is independently hydrogen.

In some embodiments, at least one R^{1C} is independently C₁-C₃ alkyl.

In some embodiments, at least one R^{1C} is independently C₁-C₃ haloalkyl.

In some embodiments, at least one R^{1C} is independently —OR^{1F}. In some embodiments, at least one R^{1C} is independently —OCH₃.

In some embodiments, at least one R^{1A} is independently —(C₁-C₃ alkylene)-OR^{1B}.

In some embodiments, at least one R^{1A} is independently —(C₁-C₃ alkylene)-SR^{1B}.

In some embodiments, two instances of R¹ are taken together to form ═O.

In some embodiments, at least one R^{1B} is independently hydrogen, C₁-C₃ alkyl, or C₁-C₃ haloalkyl substituted with 0, 1, 2, 3, or 4 R^{1D}; each R^{1D} is independently halogen, —OR^{1F}, or —N(R^{1F})₂; each R^{1F} is independently hydrogen, C₁-C₃ alkyl, or C₁-C₃ haloalkyl.

In some embodiments, at least one R^{1B} is independently hydrogen.

In some embodiments, at least one R^{1B} is independently C₁-C₃ alkyl or C₁-C₃ haloalkyl substituted with 0, 1, 2, 3, or 4 R^{1D}; each R^{1D} is independently halogen, —OR^{1F}, or —N(R^{1F})₂; and each RIF is independently hydrogen, C₁-C₃alkyl, or C₁-C₃ haloalkyl.

In some embodiments, at least one R^{1B} is independently C₁-C₃ alkyl or C₁-C₃ haloalkyl substituted with 0 R^{1D}.

In some embodiments, at least one R^{1B} is independently C₁-C₃ alkyl or C₁-C₃ haloalkyl substituted with 1 R^{1D}; R^{1D} is halogen. —OR^{1F}, or —N(R^{1F})₂; and each R^{1F} is independently hydrogen, C₁-C₃ alkyl, or C₁-C₃ haloalkyl.

In some embodiments, at least one R^{1B} is independently C₁-C₃ alkyl substituted with 0, 1, 2, 3, or 4 R^{1D}; each R^{1D} is independently halogen. —OR^{1F}, or —N(R^{1F})₂; and each R^{1F} is independently hydrogen, C₁-C₃ alkyl, or C₁-C₃ haloalkyl.

In some embodiments, at least one R^{1B} is independently C₁-C₃ alkyl substituted with 0 R^{1D}.

In some embodiments, at least one R^{1B} is independently C₁-C₃ alkyl substituted with 1 R^{1D}; R^{1D} is —OR^{1F}; and R^{1F} is C₁-C₃ alkyl.

In some embodiments, at least one R^{1B} is independently C₁-C₃ haloalkyl substituted with 0, 1, 2, 3, or 4 R^{1D}; each R^{1D} is independently halogen. —OR^{1F}, or —N(R^{1F})₂; and each R^{1F} is independently hydrogen, C₁-C₃ alkyl, or C₁-C₃ haloalkyl.

In some embodiments, at least one R^{1D} is independently halogen, —OR^{1F}, or —N(R^{1F})₂.

In some embodiments, at least one R^{1D} is independently halogen.

In some embodiments, at least one R^{1D} is independently —OR^{1F} or —N(R^{1F})₂.

In some embodiments, at least one R^{1D} is independently —OR^{1F}.

In some embodiments, at least one R^{1D} is independently —OH. In some embodiments, at least one R^{1D} is independently —O(C₁-C₃ alkyl).

In some embodiments, at least one R^{1D} is independently —N(R^{1F})₂.

In some embodiments, at least one R^{1F} is independently hydrogen, C₁-C₃ alkyl, or C₁-C₃ haloalkyl.

In some embodiments, at least one R^{1F} is independently hydrogen.

In some embodiments, at least one R^{1F} is independently C₁-C₃ alkyl or C₁-C₃ haloalkyl.

In some embodiments, at least one R^{1F} is independently C₁-C₃ alkyl.

In some embodiments, at least one R^{1F} is independently C₁-C₃ haloalkyl.

In some embodiments, at least one R^{1E} is independently —(C₁-C₃ alkylene)-OR^{1B} or —OR^{1B}.

In some embodiments, at least one R^{1E} is independently —(C₁-C₃ alkylene)-OR^{1B}.

In some embodiments, at least one R^{1E} is independently —OR^{1B}.

In some embodiments, two instances of R^{1E} are taken together to form ═O.

In some embodiments, R¹ is —CH₃, —CH₂—C(CH₃)₂—CH₂OCH₃, —CH₂CH₂OH, or —CH₂CH₂OCH₃.

In some embodiments, R¹ is —CH₃. In some embodiments, R¹ is —CH₂—C(CH₃)₂—CH₂OCH₃. In some embodiments, R¹ is —CH₂CH₂OH. In some embodiments, R¹ is —CH₂CH₂OCH.

In some embodiments, R¹ is —CH₂C(═O)N(H)OCH₃.

In some embodiments, when R¹ is -L₃-(4- to 10-membered heterocyclyl), the heterocyclyl ring comprises 1, 2, or 3 ring heteroatoms independently selected from O, N, and S. In some embodiments, when R¹ is -L₃-(4- to 10-membered heterocyclyl), the heterocyclyl ring comprises 1 or 2 ring heteroatoms independently selected from O, N, and S.

In some embodiments, when R¹ is -L₃-(7- to 10-membered heterocyclyl), the heterocyclyl ring comprises 1, 2, or 3 ring heteroatoms independently selected from O, N, and S. In some embodiments, when R¹ is -L₃-(7- to 10-membered heterocyclyl), the heterocyclyl ring comprises 1 or 2 ring heteroatoms independently selected from O, N, and S.

In some embodiments, when R¹ is -L₃-(4- to 6-membered heterocyclyl), the heterocyclyl ring comprises 1, 2, or 3 ring heteroatoms independently selected from O, N, and S. In some embodiments, when R¹ is -L₃-(4- to 6-membered heterocyclyl), the heterocyclyl ring comprises 1 or 2 ring heteroatoms independently selected from O, N, and S.

In some embodiments, when R¹ is -L₃-(5- to 6-membered heterocyclyl), the heterocyclyl ring comprises 1, 2, or 3 ring heteroatoms independently selected from O, N, and S. In some embodiments, when R¹ is -L₃-(5- to 6-membered heterocyclyl), the heterocyclyl ring comprises 1 or 2 ring heteroatoms independently selected from O, N, and S.

In some embodiments, when R¹ is -L₃-(4- to 10-membered heterocyclyl), the heterocyclyl ring is selected from:

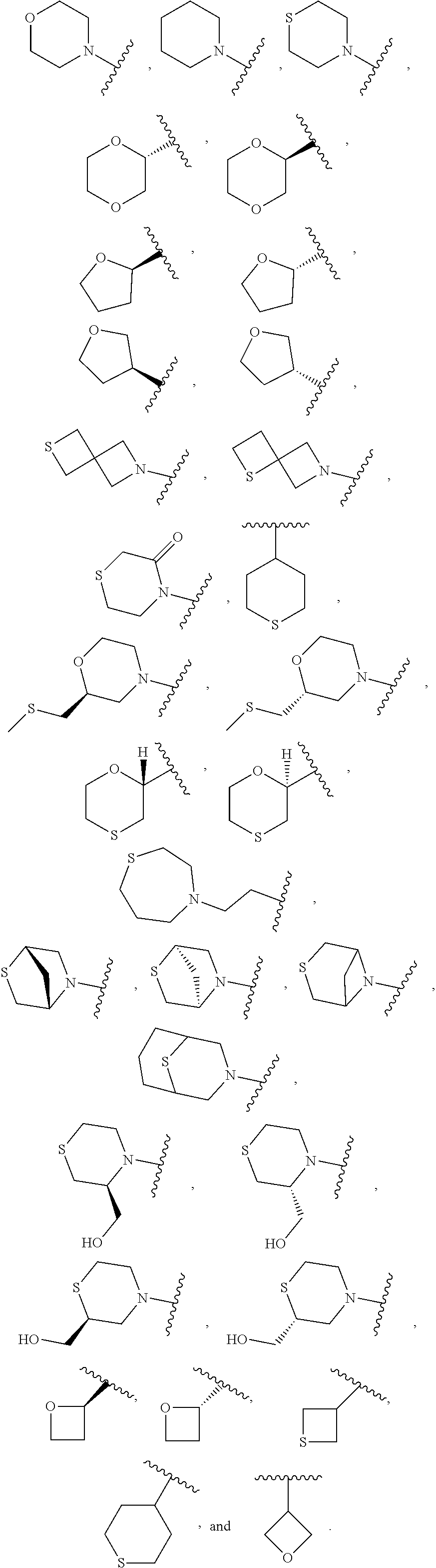

In some embodiments, when R¹ is -L₃-(C₃-C₆ carbocyclyl), the carbocyclyl ring is selected from:

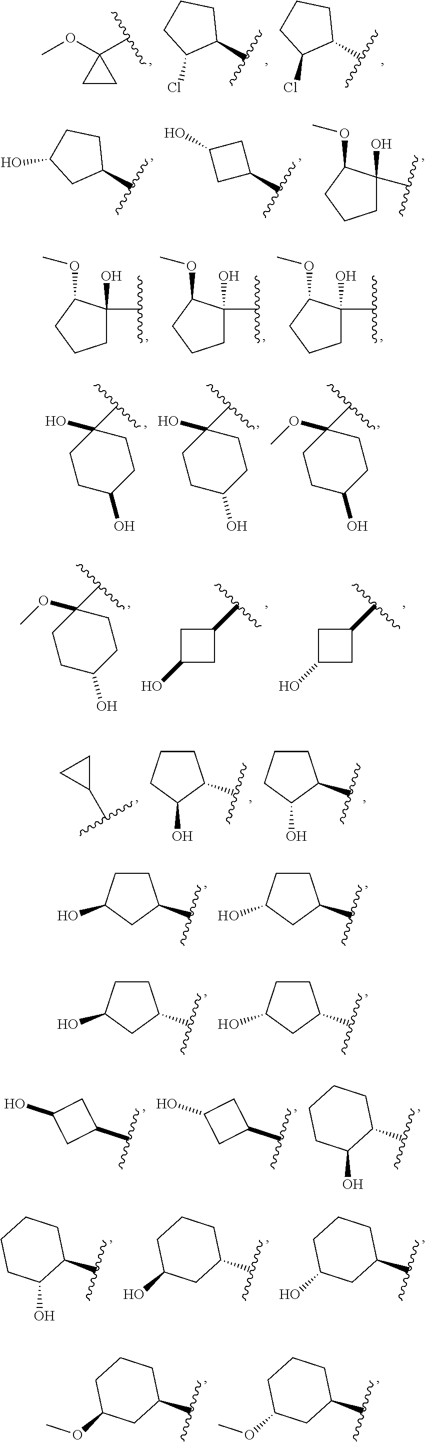

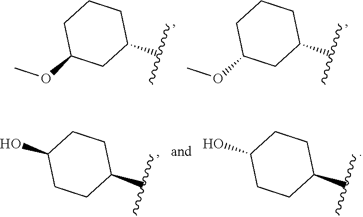

In some embodiments. R¹ is selected from:

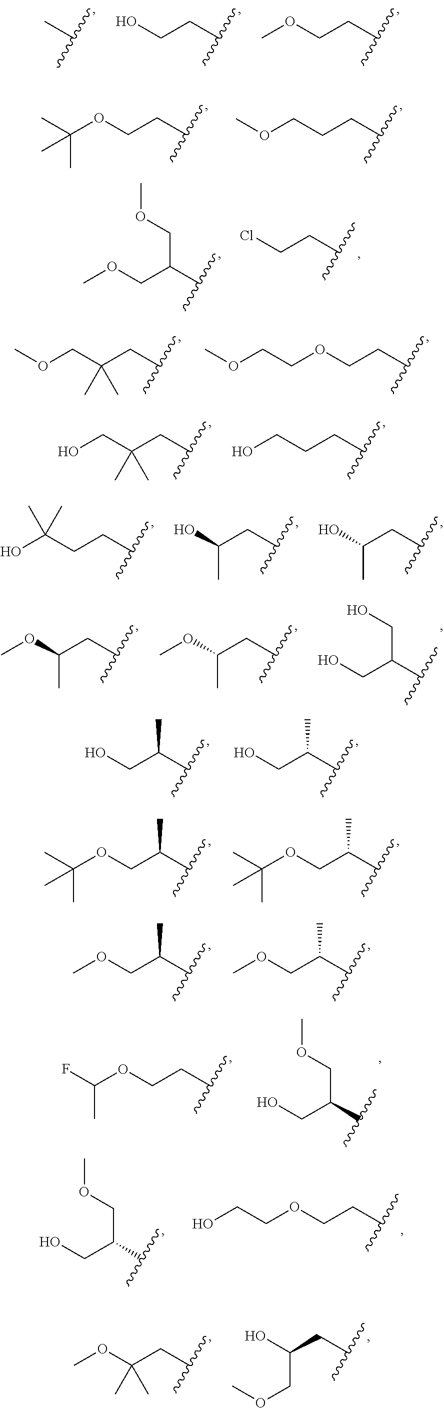

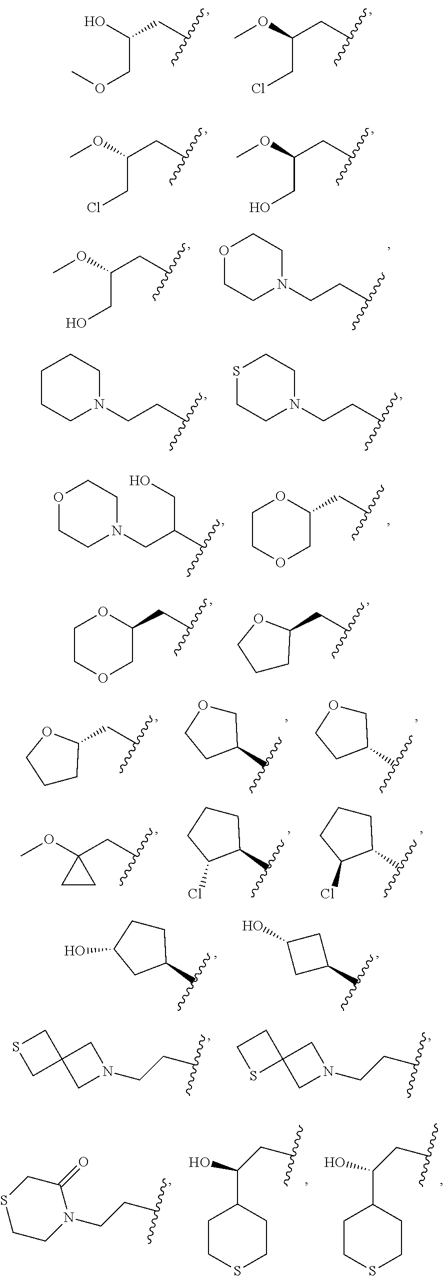

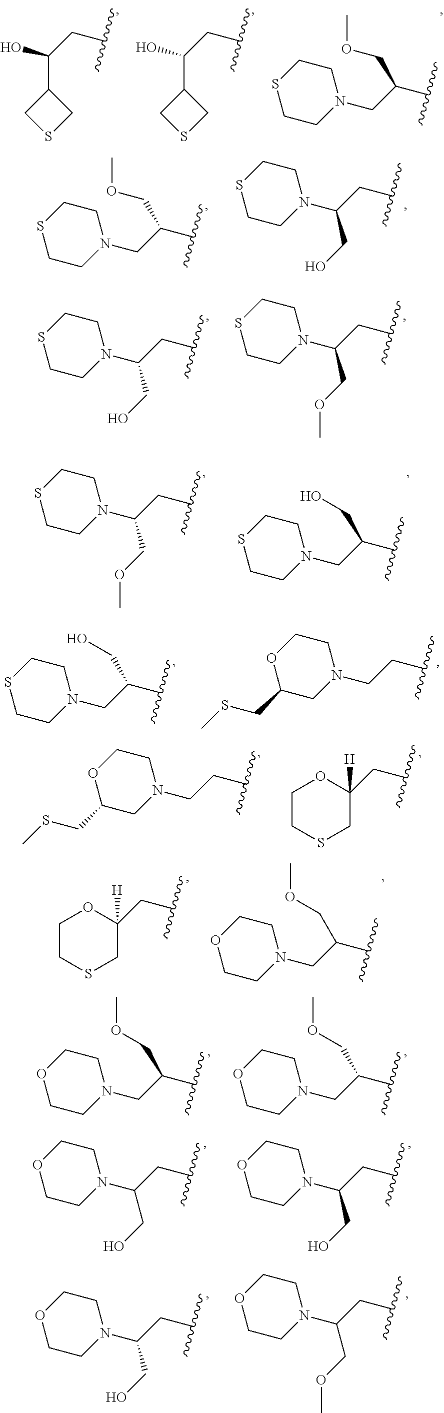

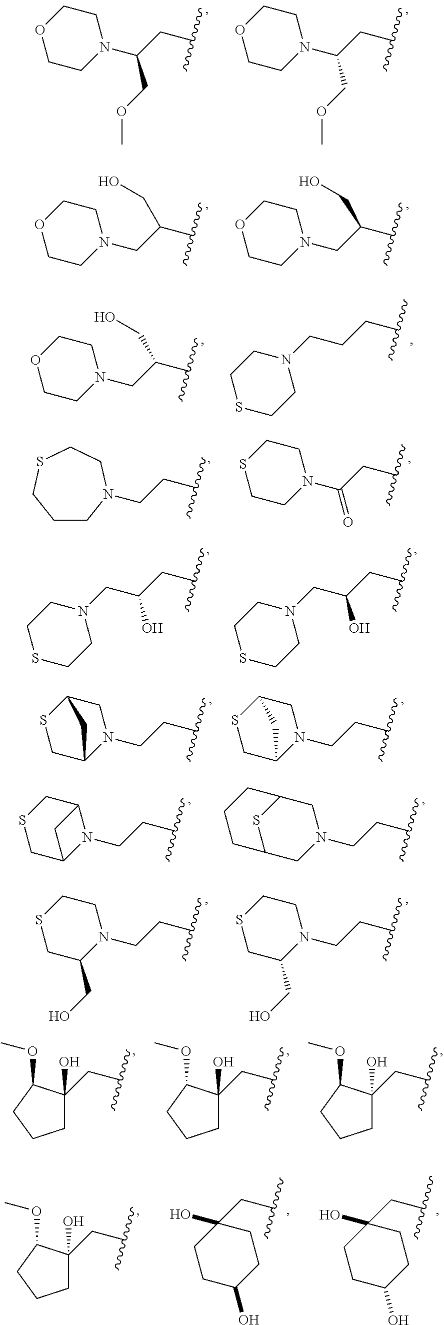

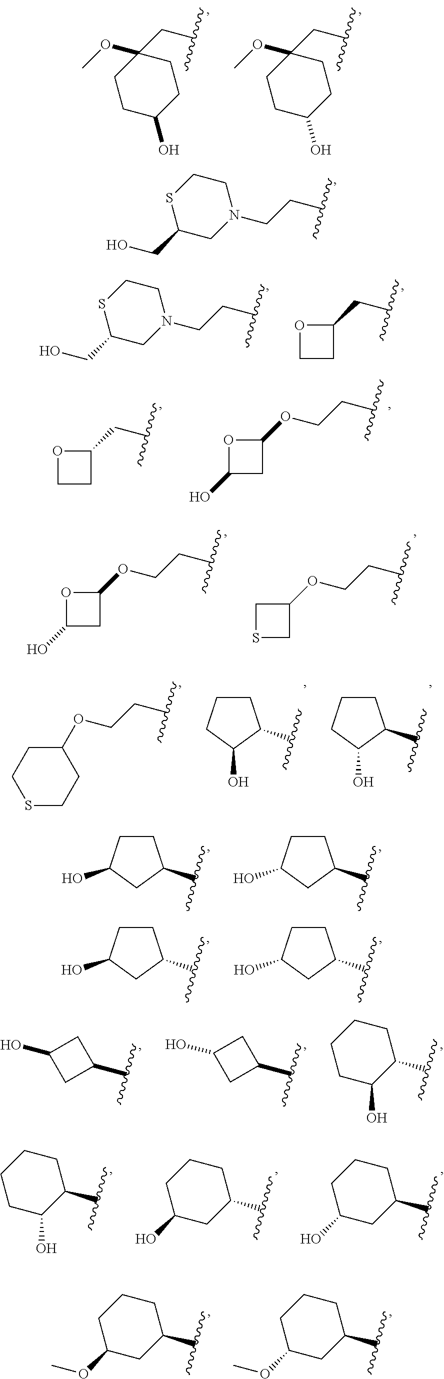

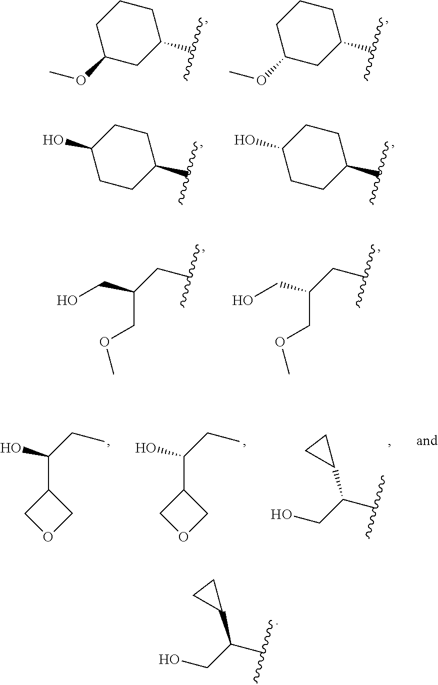

As generally described herein, Ring C is a C₆-C₁₀ aryl or 5- to 10-membered heteroaryl.

In some embodiments. Ring C is a monocyclic C₆ aryl (phenyl). In some embodiments, Ring C is phenyl, and n is 0. In some embodiments. Ring C is phenyl, and n is 1. In some embodiments. Ring C is phenyl, and n is 2. In some embodiments, Ring C is phenyl, and n is 3. In some embodiments, Ring C is phenyl, and n is 4.

In some embodiments, Ring C is of the formula (i-c):

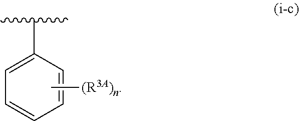

In some embodiments, Ring C is of one of the following formulae:

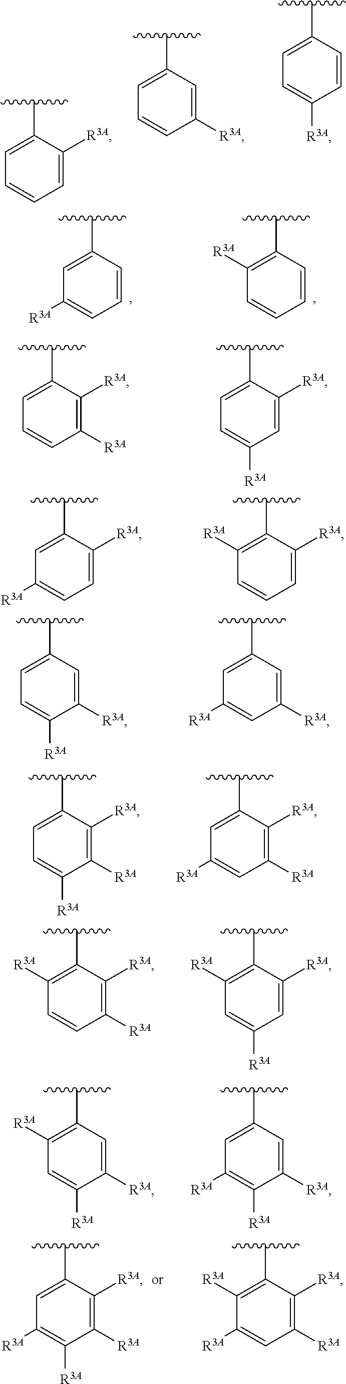

In some embodiments. Ring C is a monocyclic 5- to 6-membered heteroaryl.

In some embodiments, Ring C is 6-membered heteroaryl, wherein the heteroaryl has 1, 2, or 3 ring heteroatoms independently selected from N and O. In some embodiments, Ring C is 6-membered heteroaryl, wherein the heteroaryl has 1 or 2 ring heteroatoms independently selected from N and O. In some embodiments, Ring C is 6-membered heteroaryl, wherein the heteroaryl has 1 ring heteroatom selected from N and O. In some embodiments, Ring C is 6-membered heteroaryl, wherein the heteroaryl has 1, 2, or 3 ring N atoms. In some embodiments, Ring C is 6-membered heteroaryl, wherein the heteroaryl has 1 or 2 ring N atoms. In some embodiments. Ring C is 6-membered heteroaryl, wherein the heteroaryl has 1 ring N atom.

In some embodiments, Ring C is a pyridine ring. In some embodiments, Ring C is of the formula (ii-c):

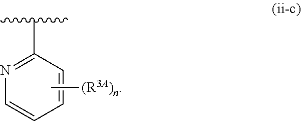

In some embodiments, Ring C is of the formula (ii-c), and n is 0. In some embodiments, Ring C is of the formula (ii-c), and n is 1. In some embodiments, Ring C is of the formula (ii-c), and n is 2. In some embodiments. Ring C is of the formula (ii-c), and n is 3. In some embodiments, Ring C is of the formula (ii-c), and n is 4.

In some embodiments. Ring C is of one of the following formulae:

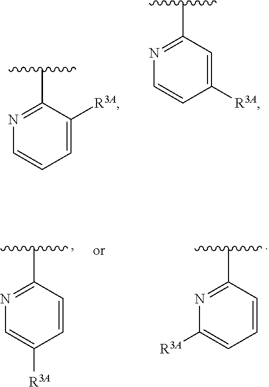

In some embodiments, Ring C is of the formula (iii-c):

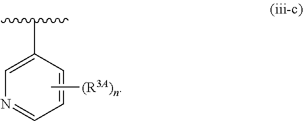

In some embodiments, Ring C is of the formula (iii-c), and n is 0. In some embodiments, Ring C is of the formula (iii-c), and n is 1. In some embodiments. Ring C is of the formula (iii-c), and n is 2. In some embodiments, Ring C is of the formula (iii-c), and n is 3. In some embodiments, Ring C is of the formula (iii-c), and n is 4.

In some embodiments. Ring C is of one of the following formulae:

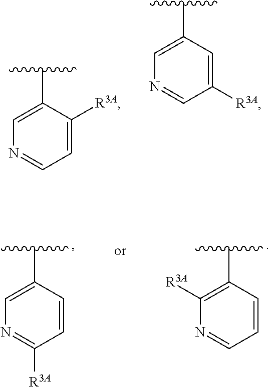

In some embodiments, Ring C is of the formula (iv-c):

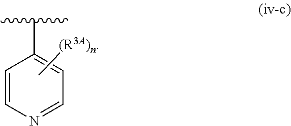

In some embodiments, Ring C is of the formula (iv-c), and n is 0. In some embodiments, Ring C is of the formula (ii-c), and n is 1. In some embodiments, Ring C is of the formula (iv-c), and n is 2. In some embodiments, Ring C is of the formula (iv-c), and n is 3. In some embodiments, Ring C is of the formula (iv-c), and n is 4.

In some embodiments. Ring C is of one of the following formulae:

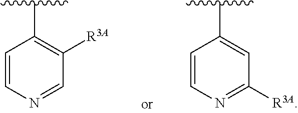

In some embodiments. Ring C is 5-membered heteroaryl. In some embodiments. Ring C is 5-membered heteroaryl, wherein the heteroaryl has 1, 2, or 3 ring heteroatoms independently selected from N, O, and S. In some embodiments, Ring C is 5-membered heteroaryl, wherein the heteroaryl has 1 or 2 ring heteroatoms independently selected from N, O, and S. In some embodiments. Ring C is 5-membered heteroaryl, wherein the heteroaryl has 1 ring heteroatom selected from N, O, and S. In some embodiments, Ring C is 5-membered heteroaryl, wherein the heteroaryl has 1, 2, or 3 ring heteroatoms independently selected from N and O. In some embodiments, Ring C is 5-membered heteroaryl, wherein the heteroaryl has 1 or 2 ring heteroatoms independently selected from N and O. In some embodiments, Ring C is 5-membered heteroaryl, wherein the heteroaryl has 1 ring heteroatom selected from N and O.

In some embodiments. Ring C is an pyrazole ring. In some embodiments, Ring C is of the formula (v-c):

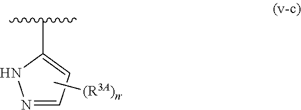

In some embodiments, Ring C is of the formula (v-c), and n is 0. In some embodiments, Ring C is of the formula (v-c), and n is 1. In some embodiments, Ring C is of the formula (v-c), and n is 2. In some embodiments, Ring C is of the formula (v-c), and n is 3.

In some embodiments, Ring C is of the formula:

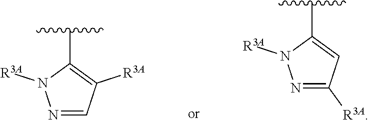

In some embodiments. Ring C is a pyrazole ring. In some embodiments, Ring C is of the formula (vi-c):

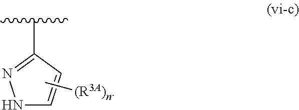

In some embodiments, Ring C is of the formula (vi-c), and n is 0. In some embodiments, Ring C is of the formula (vi-c), and n is 1. In some embodiments, Ring C is of the formula (vi-c), and n is 2. In some embodiments, Ring C is of the formula (vi-c), and n is 3.

In some embodiments, Ring C is of the formula:

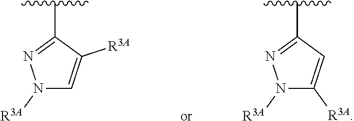

In some embodiments, compounds of Formula (I) comprise at least one R^{3A} substituent -L₁-OR^{3B}, and Ring C is substituted with 0, 1, 2, or 3 additional R^{3A} substituents. In some embodiments, the -L₁-OR^{3B} group is ortho to the point of attachment of Ring C to the pyrone ring. In certain embodiments, Ring C further comprises an additional R^{3A} group ortho to the point of attachment selected from halogen. -L₁₋OR^{3B}, or -L₁-CN.

In some embodiments, Ring C is of the formula:

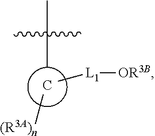

wherein n is 0, 1, 2, or 3; or

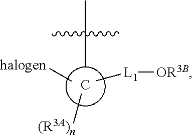

wherein n is 0, 1, or 2;

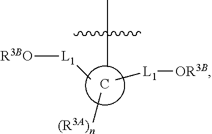

wherein n is 0, 1, or 2; or

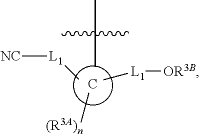

wherein n is 0, 1, or 2;
wherein the halogen, -L₁-OR^{3B}, and/or -L₁-CN groups are each ortho to the point of attachment of Ring C to the pyrone ring.

In some embodiments, compounds of Formula (I) comprise at least one R^{3A} substituent -L₁-CN, and Ring C is substituted with 0, 1, 2, or 3 additional R^{3A} substituents. In some embodiments, the -L₁-CN group is ortho to the point of attachment of Ring C to the pyrone ring. In certain embodiments, Ring C further comprises an additional R^{3A} group ortho to the point of attachment selected from halogen. -L₁₋OR^{3B}, or -L₁-CN.

In some embodiments, Ring C is of the formula:

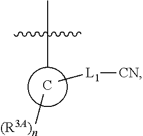

wherein n is 0, 1, 2, or 3; or

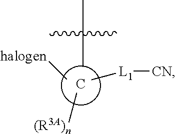

wherein n is 0, 1, or 2; or

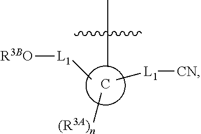

wherein n is 0, 1, or 2; or

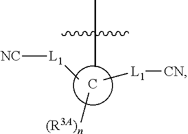

wherein n is 0, 1, or 2;
wherein the halogen, -L₁-OR^{3B}, and/or -L₁-CN groups are each ortho to the point of attachment of Ring C to the pyrone ring.

As generally defined herein, each R^{3A} is independently C₁-C₃ alkyl, C₂-C₃ alkenyl, C₂-C₃ alkynyl, halogen. -L₁-CN, -L₁-SOR^{3C}, -L₁-SO₂R^{3C}, -L₁-SR^{3B}, -L₁-PO(R^{3C})₂-L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, wherein the alkyl, alkenyl, alkynyl, carbocyclyl, heterocyclyl, aryl, and heteroaryl are independently substituted with 0, 1, 2, 3, or 4 R^{3D}.

In some embodiments, each R^{3A} is independently C₁-C₃ alkyl, C₂-C₃ alkenyl, C₂-C₃ alkynyl, halogen. -L₁-CN, -L₁-SOR^{3C}, -L₁-SO₂R^{3C}, -L₁-SR^{3B}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀ aryl), or -L₁-(5- to 10-membered heteroaryl), wherein the alkyl, alkenyl, alkynyl, carbocyclyl, heterocyclyl, aryl, and heteroaryl are independently substituted with 0, 1, 2, 3, or 4 R^{3D}.

In some embodiments, each R^{3A} is independently C₁-C₃ alkyl, halogen, -L₁-CN, -L₁-SO₂R^{3C}, -L₁-OR^{3B}, -L₁-N(R^{3B})₂, -L₁-(C₃-C₆ carbocyclyl), -L₁-(4- to 6-membered heterocyclyl), -L₁-(C₆₋₁₀aryl), or -L₁-(5- to 10-membered heteroaryl), wherein the alkyl, carbocyclyl, heterocyclyl, aryl, and heteroaryl are independently substituted with 0, 1, 2, 3, or 4 R^{3D}.

In some embodiments, at least one R^{3A} is independently C₁-C₃ alkyl substituted with 0, 1, 2, 3, or 4 R^{3D}.

In some embodiments, at least one R^{3A} is independently C₁-C₃ alkyl.

In some embodiments, at least one R^{3A} is independently C₁-C₃ alkyl substituted with 1 R^{3D}.

In some embodiments, at least one R^{3A} is independently C₁-C₃ alkyl substituted with 2 R^{3D}. In some embodiments, at least one R^{3A} is independently C₁-C₃ alkyl substituted with 3 R^{3D}.

In some embodiments, at least one R^{3A} is independently C₁-C₃ alkyl substituted with 4 R^{3D}.

In some embodiments, at least one R^{3A} is independently halogen.

In some embodiments, at least one R^{3A} is independently F or Cl.

In some embodiments, at least one R^{3A} is independently F. In some embodiments, at least one R^{3A} is independently Cl.

In some embodiments, at least one R^{3A} is independently -L₁-CN, -L₁-SO₂R^{3C}, -L₁-OR^{3B}, or -L¹-N(R^{3A})₂.

In some embodiments, at least one R^{3A} is independently -L₁-CN.

In some embodiments, at least one R^{3A} is independently —CN.

In some embodiments, at least one R^{3A} is independently —(C₁-C₃ alkylene)-CN.

In some embodiments, at least one R^{3A} is independently -L₁-SO₂R^{3C}.

In some embodiments, at least one R^{3A} is independently —SO₂R^{3C}.

In some embodiments, at least one R^{3A} is independently —(C₁-C₃ alkylene)-SO₂R^{3C}.

In some embodiments, at least one R^{3A} is independently -L₁-OR^{3B}.

In some embodiments, at least one R^{3A} is independently —OR^{3B}.

In some embodiments, at least one R^{3A} is independently -L₁-C(═O)N(R^{3B})₂ or -L₁-C(═O)OR^{3B}.

In some embodiments, at least one R^{3A} is independently —C(═O)N(R^{3B})₂ or —C(═O)OR^{3B}.

In some embodiments, at least one R^{3A} is independently —(C₁-C₃ alkylene)-OR^{3B}.

In some embodiments, at least one R^{3A} is independently -L₁-(C₃-C₆ carbocyclyl), -L₁-(4- to 6-membered heterocyclyl), -L₁-(C₆₋₁₀ aryl), or -L₁-(5- to 10-membered heteroaryl), wherein the carbocyclyl, heterocyclyl, aryl, and heteroaryl are independently substituted with 0, 1, 2, 3, or 4 R^{3D}.

In some embodiments, at least one R^{3A} is independently -L₁-(C₃-C₆ carbocyclyl), wherein the carbocyclyl is substituted with 0, 1, 2, 3, or 4 R^{3D}.

In some embodiments, at least one R^{3A} is independently -L₁-(C₃-C₆ carbocyclyl).

In some embodiments, at least one R^{3A} is independently -L₁-(C₃-C₆ carbocyclyl), wherein the carbocyclyl is substituted with 1 R^{3D}.

In some embodiments, at least one R^{3A} is independently -L₁-(C₃-C₆ carbocyclyl), wherein the carbocyclyl is substituted with 2 R^{3A}. In some embodiments, at least one R^{3A} is independently -L₁-(C₃-C₆ carbocyclyl), wherein the carbocyclyl is substituted with 3 R^{3D}. In some embodiments, at least one R^{3A} is independently -L₁-(C₃-C₆ carbocyclyl), wherein the carbocyclyl is substituted with 4 R^{3D}.

In some embodiments, at least one R^{3A} is independently —(C₃-C₆ carbocyclyl), wherein the carbocyclyl is substituted with 0, 1, 2, 3, or 4 R^{3D}.

In some embodiments, at least one R^{3A} is independently —(C₃-C₆ carbocyclyl).

In some embodiments, at least one R^{3A} is independently —(C₃-C₆ carbocyclyl), wherein the carbocyclyl is substituted with 1 R^{3D}.

In some embodiments, at least one R^{3A} is independently —(C₃-C₆ carbocyclyl), wherein the carbocyclyl is substituted with 2 R^{3D}. In some embodiments, at least one R^{3D} is independently —(C₃-C₆ carbocyclyl), wherein the carbocyclyl is substituted with 3 R^{3D}. In some embodiments, at least one R^{3A} is independently —(C₃-C₆ carbocyclyl), wherein the carbocyclyl is substituted with 4 R^{3D}.

In some embodiments, at least one R^{3A} is independently —(C₁-C₃ alkylene)-(C₃-C₆ carbocyclyl), wherein the carbocyclyl is substituted with 0, 1, 2, 3, or 4 R^{3D}.

In some embodiments, at least one R^{3A} is independently —(C₁-C₃ alkylene)-(C₃-C₆ carbocyclyl).

In some embodiments, at least one R^{3A} is independently —(C₁-C₃ alkylene)-(C₃-C₆ carbocyclyl), wherein the carbocyclyl is substituted with 1 R^{3D}.

In some embodiments, at least one R^{3A} is independently —(C₁-C₃ alkylene)-(C₃-C₆ carbocyclyl), wherein the carbocyclyl is substituted with 2 R^{3D}. In some embodiments, at least one R^{3A} is independently —(C₁-C₃ alkylene)-(C₃-C₆ carbocyclyl), wherein the carbocyclyl is substituted with 3 R^{3D}. In some embodiments, at least one R^{3A} is independently —(C₁-C₃ alkylene)-(C₁-C₆ carbocyclyl), wherein the carbocyclyl is substituted with 4 R^{3D}.

In some embodiments, at least one R^{3A} is independently -L₁-(4- to 6-membered heterocyclyl), wherein the heterocyclyl is substituted with 0, 1, 2, 3, or 4 R^{3D}.

In some embodiments, at least one R^{3A} is independently -L₁-(4- to 6-membered heterocyclyl).

In some embodiments, at least one R^{3A} is independently -L₁-(4- to 6-membered heterocyclyl), wherein the heterocyclyl is substituted with 1 R^{3D}.

In some embodiments, at least one R^{3A} is independently -L₁-(4- to 6-membered heterocyclyl), wherein the heterocyclyl is substituted with 2 R^{3D}. In some embodiments, at least one R^{3A} is independently -L₁-(4- to 6-membered heterocyclyl), wherein the heterocyclyl is substituted with 3 R^{3D}. In some embodiments, at least one R^{3A} is independently -L₁-(4- to 6-membered heterocyclyl), wherein the heterocyclyl is substituted with 4 R^{3D}.

In some embodiments, at least one R^{3A} is independently -(4- to 6-membered heterocyclyl), wherein the heterocyclyl is substituted with 0, 1, 2, 3, or 4 R^{1D}.

In some embodiments, at least one R^{3A} is independently -(4- to 6-membered heterocyclyl).

In some embodiments, at least one R^{3D} is independently -(4- to 6-membered heterocyclyl), wherein the heterocyclyl is substituted with 1 R^{3D}.

In some embodiments, at least one R^{3A} is independently -(4- to 6-membered heterocyclyl), wherein the heterocyclyl is substituted with 2 R^{3D}. In some embodiments, at least one R^{3D} is independently -(4- to 6-membered heterocyclyl), wherein the heterocyclyl is substituted with 3 R^{3D}.

In some embodiments, at least one R^{3A} is independently -(4- to 6-membered heterocyclyl), wherein the heterocyclyl is substituted with 4 R^{3D}.

In some embodiments, at least one R^{3A} is independently —(C₁-C₃ alkylene)-(4- to 6-membered heterocyclyl), wherein the heterocyclyl is substituted with 0, 1, 2, 3, or 4 R^{3D}.

In some embodiments, at least one R^{3A} is independently —(C₁-C₃ alkylene)-(4- to 6-membered heterocyclyl).

In some embodiments, at least one R^{3A} is independently —(C₁-C₃ alkylene)-(4- to 6-membered heterocyclyl), wherein the heterocyclyl is substituted with 1 R^{3D}.

In some embodiments, at least one R^{3A} is independently —(C₁-C₃ alkylene)-(4- to 6-membered heterocyclyl), wherein the heterocyclyl is substituted with 2 R^{3D}. In some embodiments, at least one R^{3A} is independently —(C₁-C₃ alkylene)-(4- to 6-membered heterocyclyl), wherein the heterocyclyl is substituted with 3 R^{3D}. In some embodiments, at least one R^{3A} is independently —(C₁-C₃ alkylene)-(4- to 6-membered heterocyclyl), wherein the heterocyclyl is substituted with 4 R^{3D}.

In some embodiments, at least one R^{3A} is independently -L₁-(C₆₋₁₀ aryl) substituted with 0, 1, 2, 3, or 4 R^{3D}.

In some embodiments, at least one R^{3A} is independently -L₁-(C₆ aryl) substituted with 0, 1, 2, 3, or 4 R^{3D}.

In some embodiments, at least one R^{3A} is independently -L₁-(C₆ aryl).

In some embodiments, at least one R^{3A} is independently -L₁-(C₆ aryl) substituted with 1 R^{3D}.

In some embodiments, at least one R^{3A} is independently -L₁-(C₆ aryl) substituted with 2 R^{3D}. In some embodiments, at least one R^{3A} is independently -L₁-(C₆ aryl) substituted with 3 R^{3D}. In some embodiments, at least one R^{3A} is independently -L₁-(C₆ aryl) substituted with 4 R^{3D}.

In some embodiments, at least one R^{3A} is independently —(C₆ aryl) substituted with 0, 1, 2, 3, or 4 R^{3D}.

In some embodiments, at least one R^{3A} is independently —(C₆ aryl).

In some embodiments, at least one R^{3A} is independently —(C₆ aryl) substituted with 1 R^{3D}.

In some embodiments, at least one R^{3A} is independently —(C₆ aryl) substituted with 2 R^{3D}. In some embodiments, at least one R^{3A} is independently —(C₆ aryl) substituted with 3 R^{3D}. In some embodiments, at least one R^{3A} is independently —(C₆ aryl) substituted with 4 R^{3D}.

In some embodiments, at least one R^{3A} is independently —(C₁-C₃ alkylene)-(C₆ aryl) substituted with 0, 1, 2, 3, or 4 R^{3D}.

In some embodiments, at least one R^{3A} is independently —(C₁-C₃ alkylene)-(C₆ aryl).

In some embodiments, at least one R^{3D} is independently —(C₁-C₃ alkylene)-(C₆ aryl) substituted with 1 R^{3D}.

In some embodiments, at least one R^{3A} is independently —(C₁-C₃ alkylene)-(C₆ aryl) substituted with 2 R^{3D}. In some embodiments, at least one R^{3A} is independently —(C₁-C₃ alkylene)-(C₆ aryl) substituted with 3 R^{3D}. In some embodiments, at least one R^{3D} is independently —(C₁-C₃ alkylene)-(C₆ aryl) substituted with 4 R^{3D}.

In some embodiments, at least one R^{3A} is independently -L₁-(5- to 10-membered heteroaryl) substituted with 0, 1, 2, 3, or 4 R^{3D}.

In some embodiments, at least one R^{3A} is independently -L₁-(5- to 10-membered heteroaryl).

In some embodiments, at least one R^{3A} is independently -L₁-(5- to 10-membered heteroaryl) substituted with 1 R^{3D}.

In some embodiments, at least one R^{3A} is independently -L₁-(5- to 10-membered heteroaryl) substituted with 2 R^{3D}. In some embodiments, at least one R^{3A} is independently -L₁-(5- to 10-membered heteroaryl) substituted with 3 R^{3D}. In some embodiments, at least one R^{3A} is independently -L₁-(5- to 10-membered heteroaryl) substituted with 4 R^{3D}.

In some embodiments, at least one R^{3A} is independently -(5- to 10-membered heteroaryl) substituted with 0, 1, 2, 3, or 4 R^{3D}.

In some embodiments, at least one R^{3A} is independently -(5- to 10-membered heteroaryl).

In some embodiments, at least one R^{3A} is independently -(5- to 10-membered heteroaryl) substituted with 1 R^{3D}.

In some embodiments, at least one R^{3A} is independently -(5- to 10-membered heteroaryl) substituted with 2 R^{3D}. In some embodiments, each R^{3A} is independently -(5- to 10-membered heteroaryl) substituted with 3 R^{3D}. In some embodiments, at least one R^{3D} is independently -(5- to 10-membered heteroaryl) substituted with 4 R^{3D}.

In some embodiments, at least one R^{3A} is independently —(C₁-C₃ alkylene)-(5- to 10-membered heteroaryl) substituted with 0, 1, 2, 3, or 4 R^{3D}.

In some embodiments, at least one R^{3A} is independently —(C₁-C₃ alkylene)-(5- to 10-membered heteroaryl).

In some embodiments, at least one R^{3A} is independently —(C₁-C₃ alkylene)-(5- to 10-membered heteroaryl) substituted with 1 R^{3D}.

In some embodiments, at least one R^{3A} is independently —(C₁-C₃ alkylene)-(5- to 10-membered heteroaryl) substituted with 2 R^{3D}. In some embodiments, at least one R^{3A} is independently —(C₁-C₃ alkylene)-(5- to 10-membered heteroaryl) substituted with 3 R^{3D}. In some embodiments, at least one R^{3A} is independently —(C₁-C₃ alkylene)-(5- to 10-membered heteroaryl) substituted with 4 R^{3D}.

In some embodiments, two R^{3A} groups are joined, with the atoms to which they are attached, to form C₆ aryl, 5- to 6-membered heteroaryl, C₃-C₆ carbocyclyl, or 4- to 6-membered heterocyclyl.

In some embodiments, two R^{3A} groups are joined, with the atoms to which they are attached, to form C₆ aryl.

In some embodiments, two R^{3A} groups are joined, with the atoms to which they are attached, to form 5- to 6-membered heteroaryl.

In some embodiments, two R^{3A} groups are joined, with the atoms to which they are attached, to form C₃-C₆ carbocyclyl.

In some embodiments, two R^{3A} groups are joined, with the atoms to which they are attached, to form 4- to 6-membered heterocyclyl.

As generally defined herein, each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D}.

In some embodiments, at least one R^{3B} is independently hydrogen.

In some embodiments, each R^{3B} is independently C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D}.

In some embodiments, each R³¹ is independently C₁-C₃ alkyl, C₃-C₆ carbocyclyl, or 4- to 6-membered heterocyclyl.

In some embodiments, at least one R^{3B} is independently C₁-C₃ alkyl substituted with 0, 1, 2, 3, or 4 R^{3D}.

In some embodiments, at least one R^{3B} is independently C₁-C₃ alkyl.

In some embodiments, at least one R^{3B} is independently C₁-C₃ alkyl substituted with 1 R^{3D}.

In some embodiments, at least one R^{3B} is independently C₁-C₃ alkyl substituted with 2 R^{3D}.

In some embodiments, at least one R^{3B} is independently C₁-C₃ alkyl substituted with 3 R^{3D}.

In some embodiments, at least one R^{3B} is independently C₁-C₃ alkyl substituted with 4 R^{3D}.

In some embodiments, at least one R^{3B} is independently C₃-C₆ carbocyclyl substituted with 0, 1, 2, 3, or 4 R^{3D}.

In some embodiments, at least one R^{3B} is independently C₃-C₆ carbocyclyl.

In some embodiments, at least one R^{3B} is independently C₃-C₆ carbocyclyl substituted with 1 R^{3D}.

In some embodiments, at least one R^{3B} is independently C₃-C₆ carbocyclyl substituted with 2 R^{3D}.

In some embodiments, at least one R^{3B} is independently C₃-C₆ carbocyclyl substituted with 3 R^{3D}.

In some embodiments, at least one R^{3B} is independently C₃-C₆ carbocyclyl substituted with 4 R^{3D}.

In some embodiments, at least one R^{3B} is independently 4- to 6-membered heterocyclyl substituted with 0, 1, 2, 3, or 4 R^{3D}.

In some embodiments, at least one R^{3B} is independently 4- to 6-membered heterocyclyl.

In some embodiments, at least one R^{3B} is independently 4- to 6-membered heterocyclyl substituted with 1 R^{3D}.

In some embodiments, at least one R^{3B} is independently 4- to 6-membered heterocyclyl substituted with 2 R^{3D}.

In some embodiments, at least one R^{3B} is independently 4- to 6-membered heterocyclyl substituted with 3 R^{3D}.

In some embodiments, at least one R^{3B} is independently 4- to 6-membered heterocyclyl substituted with 4 R^{3D}.

As generally defined herein, each R^{3C} is independently C₁-C₃ alkyl or C₁-C₃ haloalkyl.

In some embodiments, at least one R^{3C} is independently C₁-C₃ alkyl.

In some embodiments, at least one R^{3C} is independently C₁-C₃ haloalkyl.

As generally defined herein, each R^{3C} is independently halogen, —OR^{3E}, —CN, C₁-C₃ alkyl, or C₁-C₃ haloalkyl.

In some embodiments, each R^{3D} is independently halogen or —OC₁-C₃ alkyl.

In some embodiments, at least one R^{3D} is independently halogen.

In some embodiments, at least one R^{3D} is independently F or Cl.

In some embodiments, at least one R^{3D} is independently F. In some embodiments, at least one R^{3D} is independently Cl.

In some embodiments, at least one R^{3D} is independently —OR^{3E}.

In some embodiments, at least one R^{3D} is independently —OC₁-C₃ alkyl.

In some embodiments, at least one R^{3D} is independently —CN.

As generally defined herein, each R^{3E} is independently hydrogen, C₁-C₄ alkyl, or C₁-C₄ haloalkyl.

In some embodiments, at least one R^{3E} is independently hydrogen.

In some embodiments, at least one R^{3E} is independently C₁-C₃ alkyl.

In some embodiments, at least one R^{3E} is independently C₁-C₃ haloalkyl.

As generally defined herein, each L₁ is independently a bond, C₁-C₃ alkylene, or C₁-C₃ haloalkylene.

In some embodiments, each L₁ is independently a bond or C₁-C₃ alkylene.

In some embodiments, at least one L₁ is independently a bond.

In some embodiments, at least one L₁ is independently C₁-C₃ alkylene.

In some embodiments, at least one L₁ is independently branched C₁-C₃ alkylene.

In some embodiments, at least one L₁ is independently C₁ alkylene. In some embodiments, at least one L₁ is independently C₂ alkylene. In some embodiments, at least one L₁ is independently C₃ alkylene.

In some embodiments, at least one R^{3A} is independently —CN, —SO₂CH₃, —PO(CH₃)₂, —OCH₃, —OCHF₂, —CH₃, F, Cl, —OCH₂CH₂OCH₃, —CH₂CN, —CH₂OH, —CH₂OCH₃, or —OCH₂CH₂CN. In some embodiments, at least one R^{3A} is independently —CN, —OCH₃, —OCHF₂, —CH₃, or Cl. In some embodiments, at least one R^{3A} is independently —CN. In some embodiments, at least one R^{3A} is independently —SO₂CH₃. In some embodiments, at least one R^{3A} is independently —PO(CH₃)₂. In some embodiments, at least one R^{3A} is independently —OCH₃. In some embodiments, at least one R³ is independently —OCHF₂. In some embodiments, at least one R^{3A} is independently —CH₃. In some embodiments, at least one R^{3A} is independently F. In some embodiments, at least one R^{3A} is independently Cl. In some embxxiiments, at least one R^{3A} is independently —OCH₂CH₂OCH₃. In some embodiments, at least one R^{3A} is independently —CH₂CN. In some embodiments, at least one R^{3A} is independently —CH₂OH. In some embodiments, at least one R^{3A} is independently —CH₂OCH₃. In some embodiments, at least one R^{3A} is independently —OCH₂CH₂CN.

In some embodiments. Ring C is:

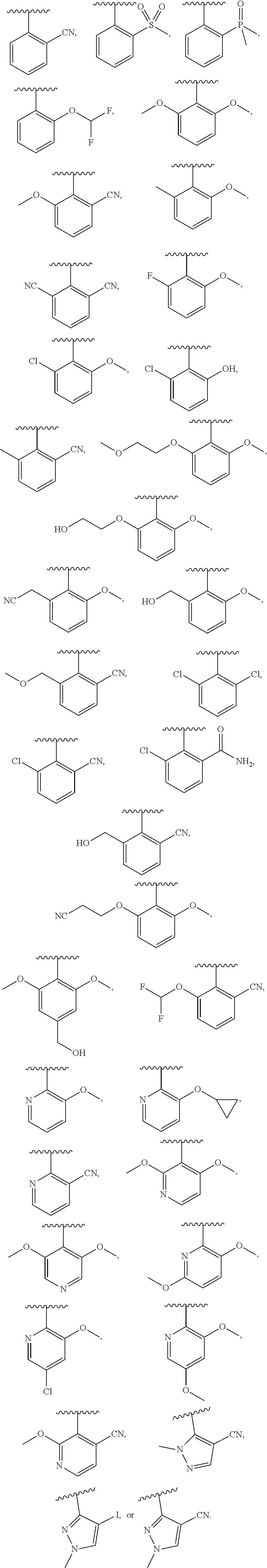

(b) Ring A. R⁴, L₂, and m

As generally defined herein, Ring A is a 5-membered monocyclic heteroaryl.

In some embodiments, Ring A is a 5-membered monocyclic heteroaryl comprising 1 nitrogen atom.

In some embodiments, Ring A is a 5-membered monocyclic heteroaryl comprising 2 nitrogen atoms.

In some embodiments, Ring A is a 5-membered monocyclic heteroaryl comprising 1 oxygen atom.

In some embodiments. Ring A is a 5-membered monocyclic heteroaryl comprising 1 nitrogen atom and 1 oxygen atom.

In some embodiments. Ring A is a 5-membered monocyclic heteroaryl comprising 1 sulfur atom.

In some embodiments, Ring A is a 5-membered monocyclic heteroaryl comprising 1 nitrogen atom and 1 sulfur atom.

In some embodiments, Ring A is a pyrrole, furan, thiophene, pyrazole, imidazole, isoxazole, oxazole, isothiazole, or thiazole ring.

In some embodiments. Ring A is:

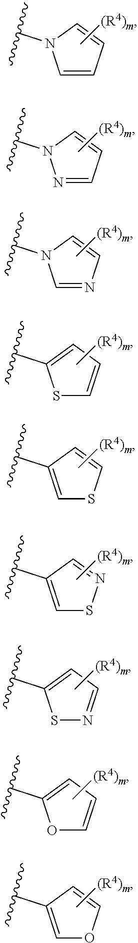

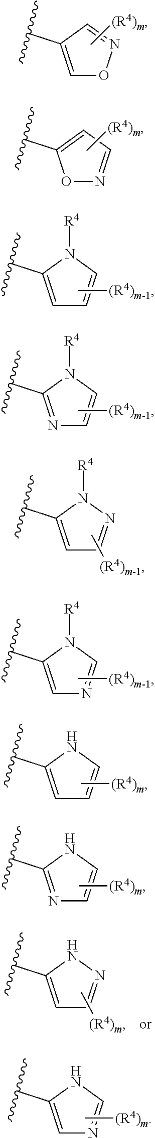

In som embodiments Ring A is:

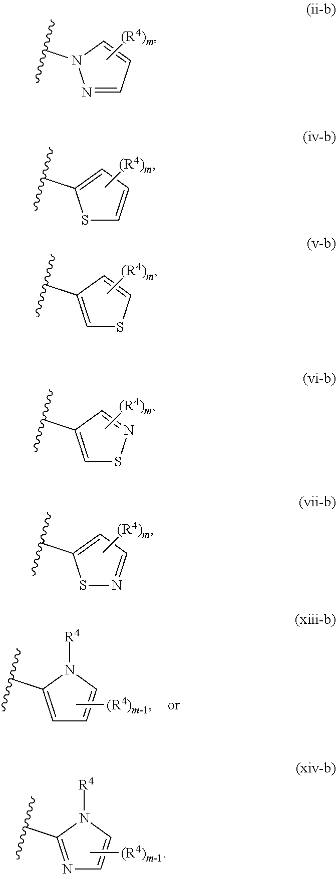

In some embodiments, Ring A is:

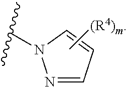

In some embodiments, Ring A is:

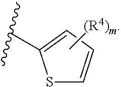

In some embodiments, Ring A is:

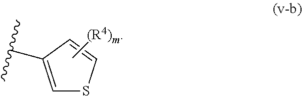

In some embodiments, Ring A is:

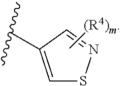

In some embodiments, Ring A is:

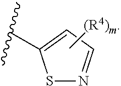

In some embodiments, Ring A is:

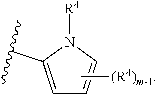

In some embodiments, Ring A is:

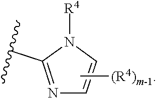

In some embodiments, Ring A is

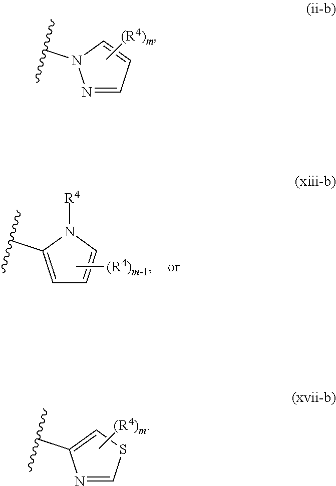

In some embodiments, Ring A is

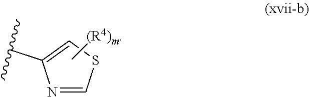

In some embodiments, Ring A is a 5-membered monocyclic heteroaryl directly linked to the thiadiazole via an N atom, as provided in formula (xviii-b):

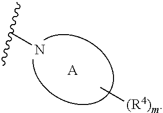

Exemplary Ring A ring systems that fall within the scope of formula (xviii-b) include, but are not limited to:

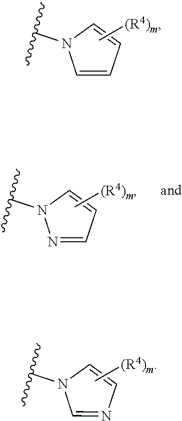

As generally defined herein, each R⁴ is independently halogen. —CN, -L₂-OR^{4A}, -L₂-N(R^{4B})₂, C₁-C₆ alkyl, or C₁-C₆ haloalkyl; each R^{4A} and R^{4B} are independently hydrogen, C₁-C₃ alkyl, C₁-C₃ haloalkyl, or —C(═O)R^{4C}, wherein R^{4C} is C₁-C₆ alkyl or C₁-C₆ haloalkyl; each L₂ is a bond, C₁-C₃ alkylene, or C₁-C₃ haloalkylene; and m is 0, 1 or 2.

In some embodiments, each R⁴ is independently halogen. —CN, -L₂-OR^{4A}, -L₂-N(R^{4B})₂, C₁-C₆ alkyl, or C₁-C₆ haloalkyl.

In some embodiments, at least one R⁴ is independently halogen.

In some embodiments, at least one R⁴ is independently —F or —Cl.

In some embodiments, at least one R⁴ is independently —F. In some embodiments, at least one R⁴ is independently —Cl.

In some embodiments, at least one R⁴ is independently —CN.

In some embodiments, at least one R⁴ is independently -L₂-OR^{4A}.

In some embodiments, at least one R⁴ is independently —OR^{4A}.

In some embodiments, at least one R⁴ is independently —(C₁-C₃ alkylene)-OR^{4A}.

In some embodiments, at least one R⁴ is independently —(C₁ alkylene)-OR^{4A}.

In some embodiments, at least one R⁴ is independently —(C₂ alkylene)-OR^{4A}.

In some embodiments, at least one R⁴ is independently —(C₃ alkylene)-OR^{4A}.

In some embodiments, at least one R⁴ is independently —OH.

In some embodiments, at least one R⁴ is independently —(C₁-C₃ alkylene)-OH.

In some embodiments, at least one R⁴ is independently —(C₁ alkylene)-OH.

In some embodiments, at least one R⁴ is independently —(C₂ alkylene)-OH.

In some embodiments, at least one R⁴ is independently —(C₃ alkylene)-OH.

In some embodiments, at least one R⁴ is independently —O(C₁-C₃ alkyl).

In some embodiments, at least one R⁴ is independently —(C₁-C₃ alkylene)-O(C₁-C₃ alkyl).

In some embodiments, at least one R⁴ is independently —(C₁ alkylene)-O(C₁-C₃ alkyl).

In some embodiments, at least one R⁴ is independently —(C₂ alkylene)-O(C₁-C₃ alkyl).

In some embodiments, at least one R⁴ is independently —(C₃ alkylene)-O(C₁-C₃ alkyl).

In some embodiments, at least one R⁴ is independently —N(R^{4B})₂.

In some embodiments, at least one R⁴ is independently -L₂-N(R^{4B})₂.

In some embodiments, at least one R⁴ is independently —(C₁-C₃ alkylene)-N(R^{4B})₂.

In some embodiments, at least one R⁴ is independently —NH₂.

In some embodiments, at least one R⁴ is independently -L₂-NH₂.

In some embodiments, at least one R⁴ is independently -L₂-NHC(O)CH₃.

In some embodiments, at least one R⁴ is independently —(C₁-C₃ alkylene)-NH₂.

In some embodiments, at least one R⁴ is independently —NH(R^{4B}).

In some embodiments, at least one R⁴ is independently -L₂-NH(R^{4B}).

In some embodiments, at least one R⁴ is independently —(C₁-C₃ alkylene)-NH(R^{4B}).

In some embodiments, at least one R⁴ is independently —N(C₁-C₃ alkyl)₂.

In some embodiments, at least one R⁴ is independently -L₂-N(C₁-C₃ alkyl)₂.

In some embodiments, at least one R⁴ is independently —(C₁-C₃ alkylene)-N(C₁-C₃ alkyl)₂.

In some embodiments, at least one R⁴ is independently C₁-C₆ alkyl.

In some embodiments, at least one R⁴ is independently methyl. In some embodiments, at least one R⁴ is independently ethyl. In some embodiments, at least one R⁴ is independently propyl. In some embodiments, at least one R⁴ is independently isopropyl. In some embodiments, at least one R⁴ is independently butyl. In some embodiments, at least one R⁴ is independently isobutyl. In some embodiments, at least one R⁴ is independently tert-butyl.

In some embodiments, at least one R⁴ is independently C₁-C₆ haloalkyl.

In some embodiments, at least one R⁴ is independently halomethyl. In some embodiments, at least one R⁴ is independently haloethyl. In some embodiments, at least one R⁴ is independently halopropyl. In some embodiments, at least one R⁴ is independently halo-isopropyl. In some embodiments, at least one R⁴ is independently halobutyl. In some embodiments, at least one R⁴ is independently halo-isobutyl. In some embodiments, at least one R⁴ is independently halo-tert-butyl.

In some embodiments, each instance of R⁴ is independently selected from the group consisting of —CH₃, —CH₂CH₃, —CHF₂, —CF₃, —C₁, —CN, —NH₂, and —CH₂OH.

In some embodiments, at least one instance of R⁴ is independently —CH₃ or —CH₂CH₃.

In some embodiments, at least one instance of R⁴ is independently —CHF₂ or —CF₃.

In some embodiments, Ring A is:

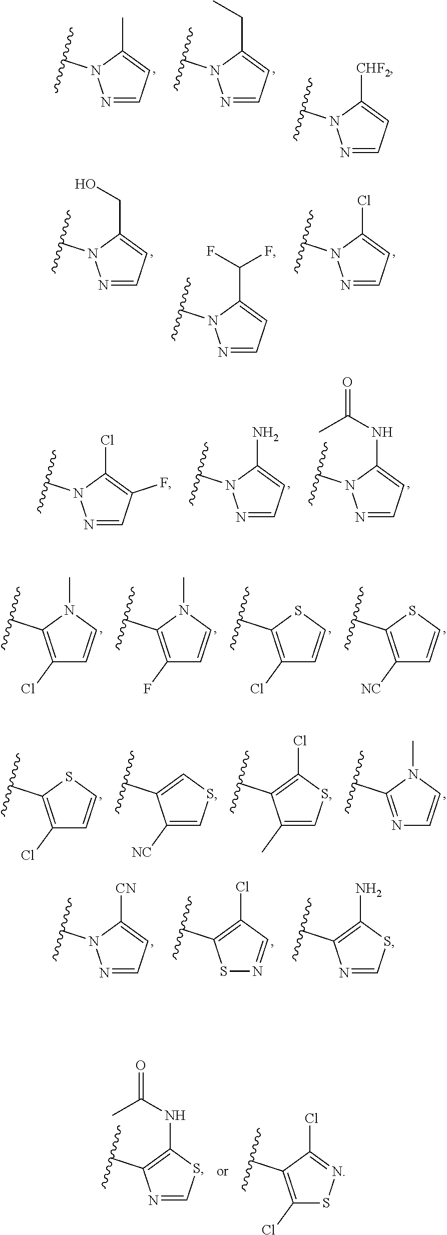

In some embodiments. Ring A ring systems of formula (xviii-b) are selected from:

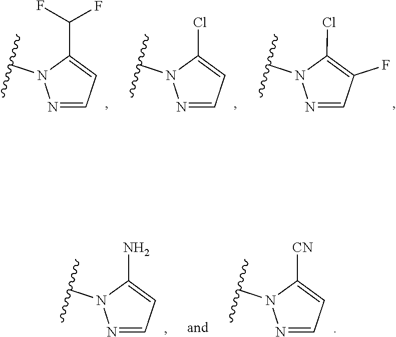

In some embodiments. Ring A is selected from:

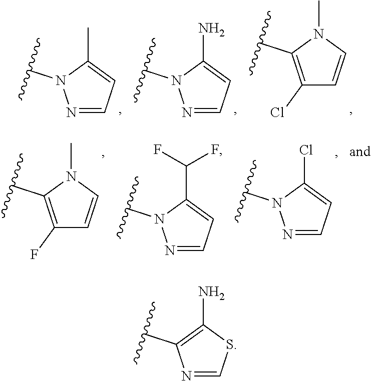

In some embodiments. Ring A is:

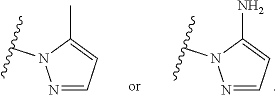

In some embodiments. Ring A is:

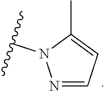 [SMILES: *n1nccc1C]

In some embodiments, Ring A is:

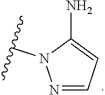 [SMILES: *n1nccc1N]

As generally defined herein, each L₂ is independently a bond. C₁-C₃ alkylene, or C₁-C₃ haloalkylene.

In some embodiments, each L₂ is independently a bond or C₁-C₃ alkylene.

In some embodiments, at least one L₂ is independently a bond.

In some embodiments, at least one L₂ is independently C₁-C₃ alkylene.

In some embodiments, at least one L₂ is independently C₁ alkylene. In some embodiments, at least one L₂ is independently C₂ alkylene. In some embodiments, at least one L₂ is independently C₃ alkylene.

As generally defined herein, m is 0, 1, or 2.

In some embodiments, m is 0.

In some embodiments, m is 1 or 2.

In some embodiments, m is 1. In some embodiments, m is 2.

### (c) Subgenera

It is understood that, for a compound of the present disclosure, variables Ring A, Ring C, R¹, R^{1A}, R^{1B}, R^{1C}, R^{1D}, R^{1E}, R^{1F}, R^{3A}, R^{3B}, R^{3C}, R^{3D}, R^{3E}, R⁴, R^{4A}, R^{4B}, L₁, L₂, L₃, m, and n can each be, where applicable, selected from the groups described herein, and any group described herein for any of variables Ring A, Ring C, R¹, R^{1A}, R^{1B}, R^{1C}, R^{1D}, R^{1E}, R^{1F}, R^{3A}, R^{3B}, R^{3C}, R^{3D}, R^{3E}, R⁴, R^{4A}, R^{4B}, L₁, L₂, L₃, m, and n can be combined, where applicable, with any group described herein for one or more of the remainder of variables Ring A, Ring C, R¹, R^{1A}, R^{1B}, R^{1C}, R^{1D}, R^{1E}, R^{1F}, R^{3A}, R^{3B}, R^{3C}, R^{3D}, R^{3E}, R⁴, R^{4A}, R^{4B}, L₁, L₂, L₃, m, and n. Additional exemplary combinations of the above described embodiments are further contemplated herein.

For example, in some embodiments, wherein Ring A is a group of formula (ii-b), the compound of Formula (I) is of Formula (I-a):

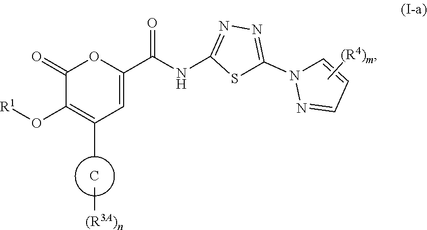

or a pharmaceutically acceptable salt thereof. In certain embodiments, R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}. In certain embodiments. R¹ is -L₃-(C₃-C₆ carbocyclyl), wherein the carbocyclyl is substituted with 1 R^{1A}; R^{1A} is —OR^{1B}; R^{1B} is hydrogen, and L₃ is bond. In certain embodiments, R¹ is -L₃-(4- to 10-membered heterocyclyl), wherein the heterocyclyl is substituted with 0 R^{1A}; and L₃ is C₁-C₃ alkylene substituted with 0 R^{1E}. In certain embodiments, Ring C is

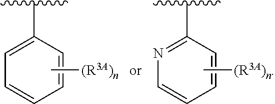

In certain embodiments, each instance of R^{3A} is independently -L₁-CN, -L₁-OR^{3B}, C₁-C₃ alkyl (e.g., —CH₃), or halo (e.g., Cl). In certain embodiments, each instance of R⁴ is independently C₁₋₃ alkyl (e.g., —CH₃) or —NH₂. In certain embodiments, m is 1 or 2. In certain embodiments, n is 1 or 2.

In some embodiments, wherein Ring A is a group of formula (xiii-b), the compound of Formula (I) is of Formula (I-b):

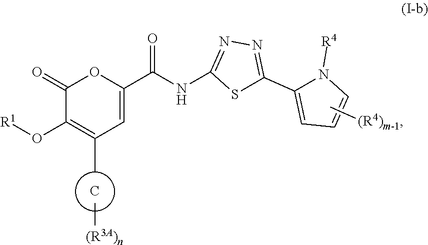

or a pharmaceutically acceptable salt thereof. In certain embodiments. R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}. In certain embodiments, R¹ is -L₃-(C₃-C₆ carbocyclyl), wherein the carbocyclyl is substituted with 1 R^{1A}; R^{1A} is —OR^{1B}; R^{1B} is hydrogen, and L₃ is bond. In certain embodiments, R¹ is -L₃-(4- to 10-membered heterocyclyl), wherein the heterocyclyl is substituted with 0 R^{1A}; and L₃ is C₁-C₃ alkylene substituted with 0 R^{1E}. In certain embodiments, Ring C is

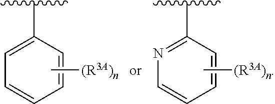

In certain embodiments, each instance of R^{3A} is independently -L₁-CN, -L₁-OR^{3B}, C₁-C₃ alkyl (e.g., —CH₃), or halo (e.g., Cl). In certain embodiments, each instance of R⁴ is independently C₁₋₃ alkyl (e.g., —CH₃) or —NH₂. In certain embodiments, m is 1 or 2. In certain embodiments, n is 1 or 2.

In some embodiments, wherein Ring A is a group of formula (xvii-b), the compound of Formula (I) is of Formula (I-c):

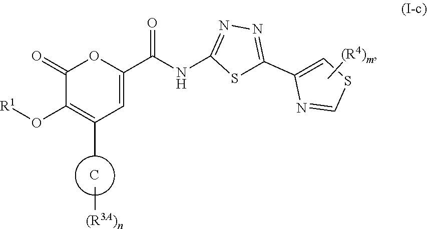

or a pharmaceutically acceptable salt thereof. In certain embodiments. R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}. In certain embodiments, R¹ is -L₃-(C₃-C₆ carbocyclyl), wherein the carbocyclyl is substituted with 1 R^{1A}; R^{1A} is —OR^{1B}; R^{1B} is hydrogen, and L₃ is bond. In certain embodiments, R¹ is -L₃-(4- to 10-membered heterocyclyl), wherein the heterocyclyl is substituted with 0 R^{1A}; and L₃ is C₁-C₃ alkylene substituted with 0 R^{1E}. In certain embodiments, Ring C is

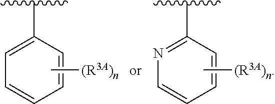

In certain embodiments, each instance of R^{3A} is independently -L₁-CN, -L₁-OR^{3B}. C₁-C₃ alkyl (e.g., —CH₃), or halo (e.g., Cl). In certain embodiments, each instance of R⁴ is independently C₁₋₃ alkyl (e.g., —CH₃) or —NH₂. In certain embodiments, m is 1 or 2. In certain embodiments, n is 1 or 2.

In some embodiments, wherein Ring C is of the formula (i-c), the compound of Formula (I) is of Formula (I-d):

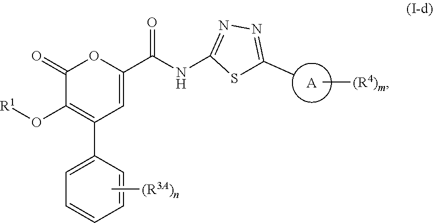

or a pharmaceutically acceptable salt thereof. In certain embodiments, R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}. In certain embodiments, R¹ is -L₃-(C₃-C₆ carbocyclyl), wherein the carbocyclyl is substituted with 1 R^{1A}; R^{1A} is —OR^{1B}; R^{1B} is hydrogen, and L₃ is bond. In certain embodiments, R¹ is -L₃-(4- to 10-membered heterocyclyl), wherein the heterocyclyl is substituted with 0 R^{1A}; and L₃ is C₁-C₃ alkylene substituted with 0 R^{1E}. In certain embodiments, each instance of R^{3A} is independently -L₁-CN, -L₁-OR^{1B}. C₁-C₃ alkyl (e.g., —CH₃), or halo (e.g., Cl). In certain embodiments, each instance of R⁴ is independently C₁₋₃ alkyl (e.g., —CH₃) or —NH₂. In certain embodiments, Ring A is

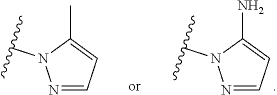

In certain embodiments, m is 1 or 2. In certain embodiments, n is 1 or 2.

In some embodiments, wherein Ring C is of the Formula (ii-c), (iii-c), or (iv-c), the compound of Formula (I) is of Formula (I-e-1), (I-e-2), or (I-e-3), respectively:

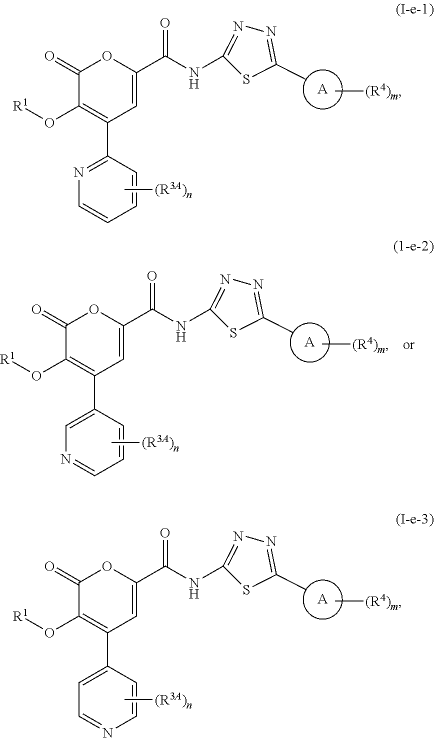

or a pharmaceutically acceptable salt thereof. In certain embodiments, R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}. In certain embodiments. R¹ is -L₃-(C₃-C₆ carbocyclyl), wherein the carbocyclyl is substituted with 1 R^{1A}; R^{1A} is —OR^{1B}; R^{1B} is hydrogen, and L₃ is bond. In certain embodiments, R¹ is -L₃-(4- to 10-membered heterocyclyl), wherein the heterocyclyl is substituted with 0 R^{1A}; and L₃ is C₁-C₃ alkylene substituted with 0 R^{1E}. In certain embodiments, each instance of R^{3A} is independently -L₁-CN. -L₁-OR^{3B}, C₁-C₃ alkyl (e.g., —CH₃), or halo (e.g., Cl). In certain embodiments, each instance of R⁴ is independently C₁₋₃ alkyl (e.g., —CH₃) or —NH₂. In certain embodiments, Ring A is

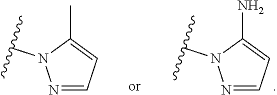

In certain embodiments, m is 1 or 2. In certain embodiments, n is 1 or 2.

In some embodiments, wherein Ring C is of the formula (v-c) or (vi-c), the compound of Formula (I) is of Formula (I-f-1) or (I-f-2), respectively:

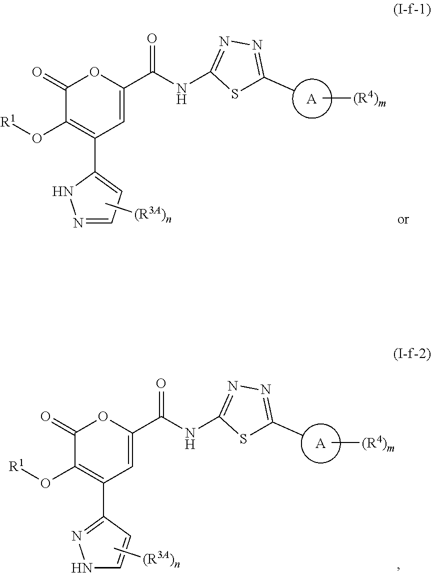

or a pharmaceutically acceptable salt thereof. In certain embodiments, R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}. In certain embodiments, R¹ is -L₃-(C₃-C₆ carbocyclyl), wherein the carbocyclyl is substituted with 1 R^{1A}; R^{1A} is —OR^{1B}; R^{1B} is hydrogen, and L₃ is bond. In certain embodiments, R¹ is -L₃-(4- to 10-membered heterocyclyl), wherein the heterocyclyl is substituted with 0 R^{1A}; and L₃ is C₁-C₃ alkylene substituted with 0 R^{1E}. In certain embodiments, each instance of R^{3A} is independently -L₁-CN, -L₁-OR^{3B}, C₁-C₃ alkyl (e.g., —CH₃), or halo (e.g., Cl). In certain embodiments, each instance of R⁴ is independently C₁₋₃ alkyl (e.g., —CH₃) or —NH₂. In certain embodiments, Ring A is

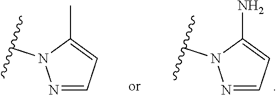

In certain embodiments, m is 1 or 2. In certain embodiments, n is 1 or 2.

In some embodiments, the compound of Formula (I) is selected from any one of the compounds of Table 1 or Table 2, or a pharmaceutically acceptable salt thereof.

In some embodiments, the compound of Formula (I) is selected from a pharmaceutically acceptable salt of any one of the compounds of Table 1 or Table 2.

In some embodiments, the compound of Formula (I) is a free base selected from any one of the compounds of Table 1 or Table 2.

The below Table 1 and Table 2 also provides the location of the Compound (#) in the Examples (Ex) by Example Number or as provided in Table A (TA) of the Examples.

TABLE 1
Compounds of Formula (I)

| # | Ex | Structure and Name |
|---|---|---|
| 1 | 1 | 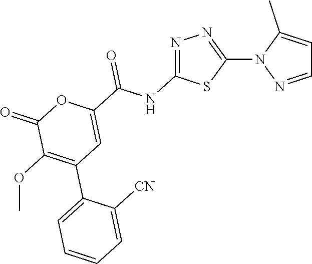 [SMILES: COc1c(-c2ccccc2C#N)cc(C(=O)Nc2nnc(-n3nccc3C)s2)oc1=O] |
| | | 4-(2-cyanophenyl)-3-methoxy-N- |
| | | (5-(5-methyl-1H-pyrazol-1-yl)- |
| | | 1,3,4-thiadiazol-2-yl)-2-oxo-2H- |
| | | pyran-6-carboxamide |
| 2 | TA | 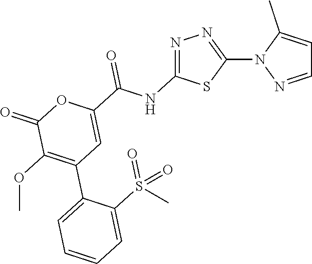 [SMILES: COc1c(-c2ccccc2S(C)(=O)=O)cc(C(=O)Nc2nnc(-n3nccc3C)s2)oc1=O] |
| | | 3-methoxy-N-(5-(5-methyl-1H- |
| | | pyrazol-1-yl)-1,3,4-thiadiazol-2- |
| | | yl)-4-(2-(methylsulfonyl)phenyl)- |
| | | 2-oxo-2H-pyran-6-carboxamide |
| 3 | 2 | 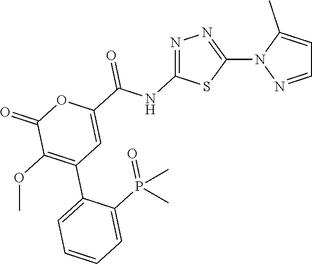 [SMILES: COc1c(-c2ccccc2P(C)(C)=O)cc(C(=O)Nc2nnc(-n3nccc3C)s2)oc1=O] |
| | | 4-(2- |
| | | (dimethylphosphoryl)phenyl)-3- |
| | | methoxy-N-(5-(5-methyl-1H- |
| | | pyrazol-1-yl)-1,3,4-thiadiazol-2- |
| | | yl)-2-oxo-2H-pyran-6- |
| | | carboxamide |
| 4 | 3 | 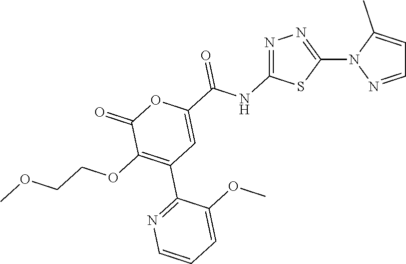 [SMILES: COCCOc1c(-c2ncccc2OC)cc(C(=O)Nc2nnc(-n3nccc3C)s2)oc1=O] |
| | | 3-(2-methoxyethoxy)-4-(3- |
| | | methoxypyridin-2-yl)-N-(5-(5- |
| | | methyl-1H-pyrazol-1-yl)-1,3,4- |
| | | thiadiazol-2-yl)-2-oxo-2H-pyran- |
| | | 6-carboxamide |
| 5 | 4 | 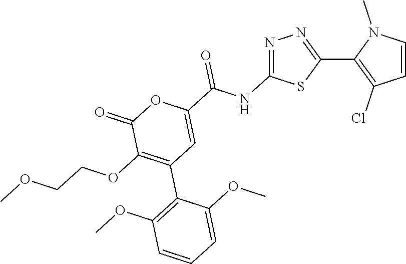 [SMILES: COCCOc1c(-c2c(OC)cccc2OC)cc(C(=O)Nc2nnc(-c3c(Cl)ccn3C)s2)oc1=O] |
| | | N-(5-(3-chloro-1-methyl-1H- |
| | | pyrrol-2-yl)-1,3,4-thiadiazol-2- |
| | | yl)-4-(2,6-dimethoxyphenyl)-3- |
| | | (2-methoxyethoxy)-2-oxo-2H- |
| | | pyran-6-carboxamide |
| 6 | 5 | 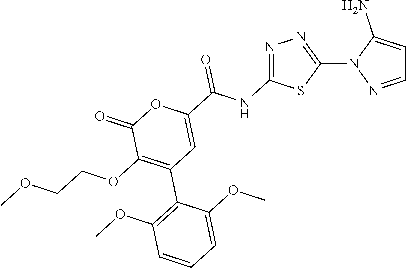 [SMILES: COCCOc1c(-c2c(OC)cccc2OC)cc(C(=O)Nc2nnc(-n3nccc3N)s2)oc1=O] |
| | | N-[5-(5-aminopyrazol-1-yl)- |
| | | 1,3,4-thiadiazol-2-yl]-4-(2,6- |
| | | dimethoxyphenyl)-5-(2- |
| | | methoxyethoxy)-6-oxopyran-2- |
| | | carboxamide |
| 6- Ac | 5 | 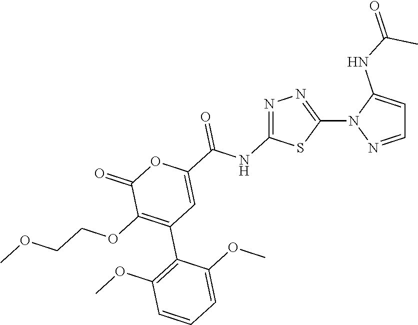 [SMILES: COCCOc1c(-c2c(OC)cccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2,6- |
| | | dimethoxyphenyl)-3-(2- |
| | | methoxyethoxy)-2-oxo-2H- |
| | | pyran-6-carboxamide |
| 7 | 6 | 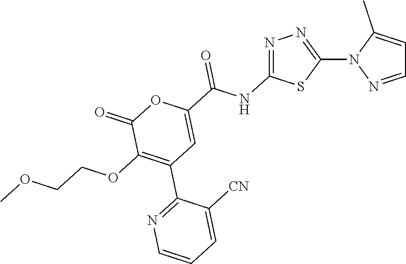 [SMILES: COCCOc1c(-c2ncccc2C#N)cc(C(=O)Nc2nnc(-n3nccc3C)s2)oc1=O] |
| | | 4-(3-cyanopyridin-2-yl)-3-(2- |
| | | methoxyethoxy)-N-(5-(5-methyl- |
| | | 1H-pyrazol-1-yl)-1,3,4- |
| | | thiadiazol-2-yl)-2-oxo-2H-pyran- |
| | | 6-carboxamide |
| 8 | 7 | 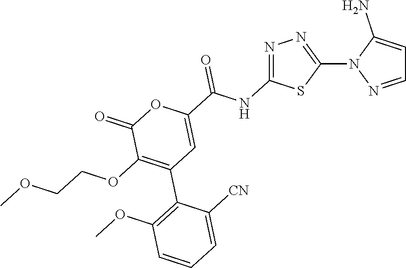 [SMILES: COCCOc1c(-c2c(C#N)cccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2-cyano- |
| | | 6-methoxyphenyl)-3-(2- |
| | | methoxyethoxy)-2-oxo-2H- |
| | | pyran-6-carboxamide |
| 8- Ac | 7 | 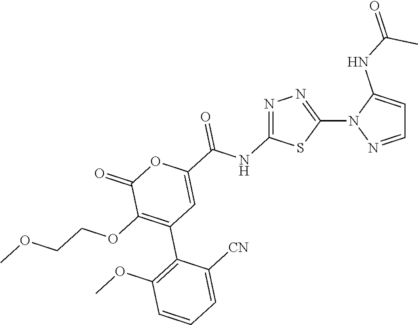 [SMILES: COCCOc1c(-c2c(C#N)cccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | cyano-6-methoxyphenyl)-3-(2- |
| | | methoxyethoxy)-2-oxo-2H- |
| | | pyran-6-carboxamide |
| 9 | 8 | 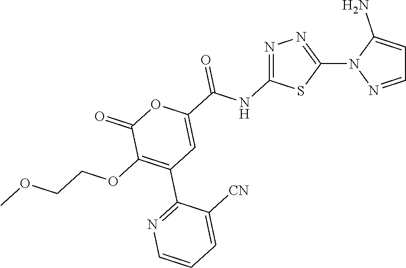 [SMILES: COCCOc1c(-c2ncccc2C#N)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(3- |
| | | cyanopyridin-2-yl)-3-(2- |
| | | methoxyethoxy)-2-oxo-2H- |
| | | pyran-6-carboxamide |
| 9- Ac | 8 | 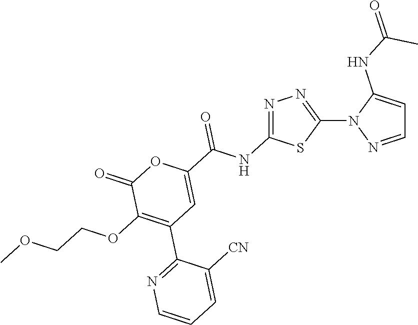 [SMILES: COCCOc1c(-c2ncccc2C#N)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(3- |
| | | cyanopyridin-2-yl)-3-(2- |
| | | methoxyethoxy)-2-oxo-2H- |
| | | pyran-6-carboxamide |
| 10 | 9 | 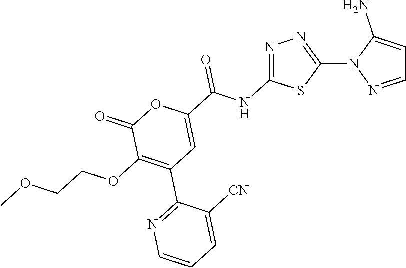 [SMILES: COCCOc1c(-c2ncccc2C#N)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-3-(2- |
| | | methoxyethoxy)-4-(3- |
| | | methoxypyridin-2-yl)-2-oxo-2H- |
| | | pyran-6-carboxamide |
| 10- Ac | 9 | 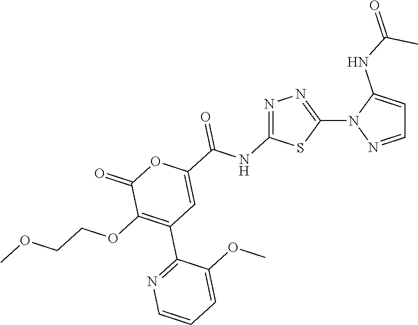 [SMILES: COCCOc1c(-c2ncccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-3-(2- |
| | | methoxyethoxy)-4-(3- |
| | | methoxypyridin-2-yl)-2-oxo-2H- |
| | | pyran-6-carboxamide |
| 11 | TA | 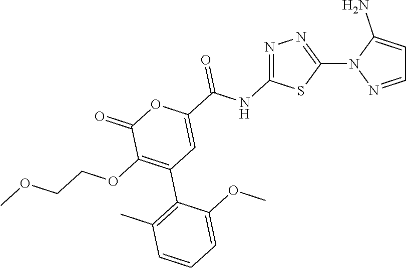 [SMILES: COCCOc1c(-c2c(C)cccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2- |
| | | methoxy-6-methylphenyl)-3-(2- |
| | | methoxyethoxy)-2-oxo-2H- |
| | | pyran-6-carboxamide |
| 12 | 10 | 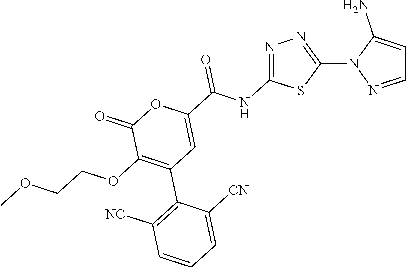 [SMILES: COCCOc1c(-c2c(C#N)cccc2C#N)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2,6- |
| | | dicyanophenyl)-3-(2- |
| | | methoxyethoxy)-2-oxo-2H- |
| | | pyran-6-carboxamide |
| 12- Ac | 10 | 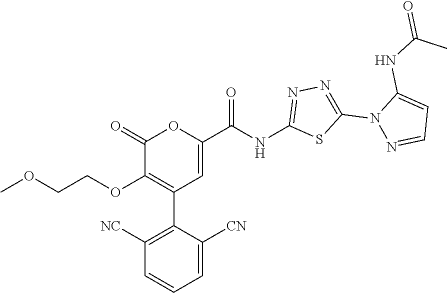 [SMILES: COCCOc1c(-c2c(C#N)cccc2C#N)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2,6- |
| | | dicyanophenyl)-3-(2- |
| | | methoxyethoxy)-2-oxo-2H- |
| | | pyran-6-carboxamide |
| 13 | TA | 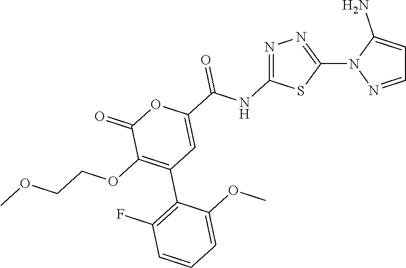 [SMILES: COCCOc1c(-c2c(F)cccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2- |
| | | fluoro-6-methoxyphenyl)-3-(2- |
| | | methoxyethoxy)-2-oxo-2H- |
| | | pyran-6-carboxamide |
| 14 | 11 | 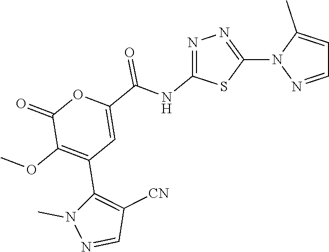 [SMILES: COc1c(-c2c(C#N)cnn2C)cc(C(=O)Nc2nnc(-n3nccc3C)s2)oc1=O] |
| | | 4-(4-cyano-1-methyl-1H-pyrazol- |
| | | 5-yl)-3-methoxy-N-(5-(5-methyl- |
| | | 1H-pyrazol-1-yl)-1,3,4- |
| | | thiadiazol-2-yl)-2-oxo-2H-pyran- |
| | | 6-carboxamide |
| 15 A* | 12 | 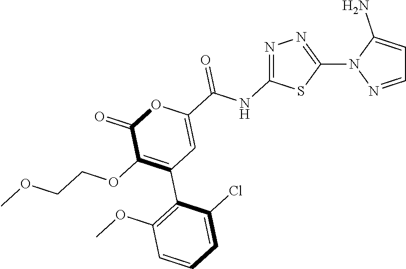 |
| | | (Ra)-N-(5-(5-amino-1H-pyazol- |
| | | 1-yl)-1,3,4-thiadiazol-2-yl)-4-(2- |
| | | chloro-6-methoxyphenyl)-3-(2- |
| | | methoxyethoxy)-2-oxo-2H- |
| | | pyran-6-carboxamide |
| 15 B* | 12 | 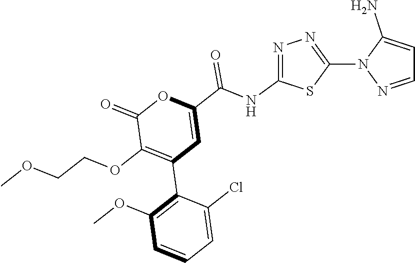 |
| | | (Sa)-N-(5-(5-amino-1H-pyrazol- |
| | | 1-yl)-1,3,4-thiadiazol-2-yl)-4-(2- |
| | | chloro-6-methoxyphenyl)-3-(2- |
| | | methoxyethoxy)-2-oxo-2H- |
| | | pyran-6-carboxamide |
| 15- Ac | 12 | 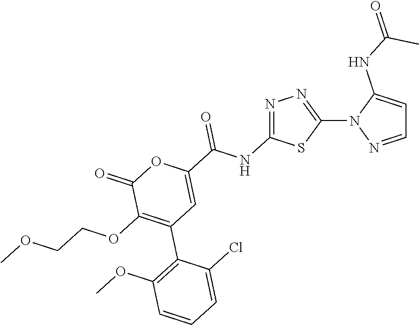 [SMILES: COCCOc1c(-c2c(Cl)cccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | chloro-6-methoxyphenyl)-3-(2- |
| | | methoxyethoxy)-2-oxo-2H- |
| | | pyran-6-carboxamide |
| 17 | 13 | 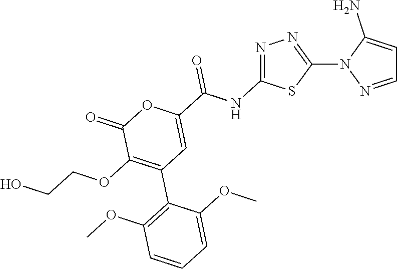 [SMILES: COc1cccc(OC)c1-c1cc(C(=O)Nc2nnc(-n3nccc3N)s2)oc(=O)c1OCCO] |
| | | N-(5-(5-amino-1H-pyrazol-1-yl)- |
| | | 1,3,4-thiadiazol-2-yl)-4-(2,6- |
| | | dimethoxyphenyl)-3-(2- |
| | | hydroxyethoxy)-2-oxo-2H-pyran- |
| | | 6-carboxamide |
| 17- Ac- OtBu | 13 | 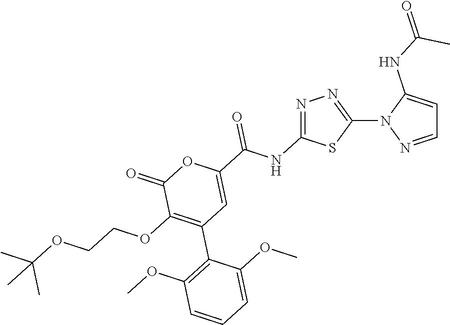 [SMILES: COc1cccc(OC)c1-c1cc(C(=O)Nc2nnc(-n3nccc3NC(C)=O)s2)oc(=O)c1OCCOC(C)(C)C] |
| | | N-(5-(5-acetamido-1H-pyrazol-1- |
| | | yl)-1,3,4-thiadiazol-2-yl)-3-(2- |
| | | (tert-butoxy)ethoxy)-4-(2,6- |
| | | dimethoxyphenyl)-2-oxo-2H- |
| | | pyran-6-carboxamide |
| 18 | 14 | 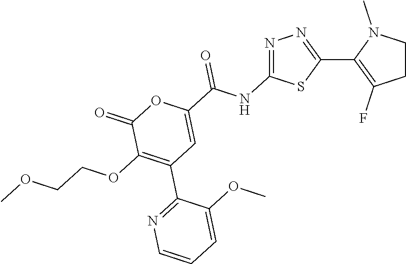 [SMILES: COCCOc1c(-c2ncccc2OC)cc(C(=O)Nc2nnc(C3=C(F)CCN3C)s2)oc1=O] |
| | | N-(5-(3-fluoro-1-methyl-1H- |
| | | pyrrol-2-yl)-1,3,4-thiadiazol-2- |
| | | yl)-3-(2-methoxyethoxy)-4-(3- |
| | | methoxypyridin-2-yl)-2-oxo-2H- |
| | | pyran-6-carboxamide |
| 19 | 15 | 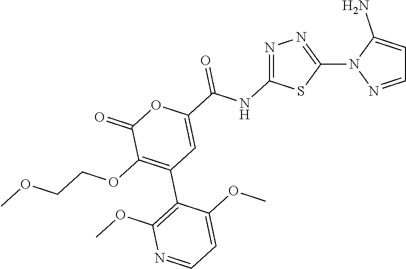 [SMILES: COCCOc1c(-c2c(OC)ccnc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2,4- |
| | | dimethoxypyridin-3-yl)-3-(2- |
| | | methoxyethoxy)-2-oxo-2H- |
| | | pyran-6-carboxamide |
| 19- Ac | 15 | 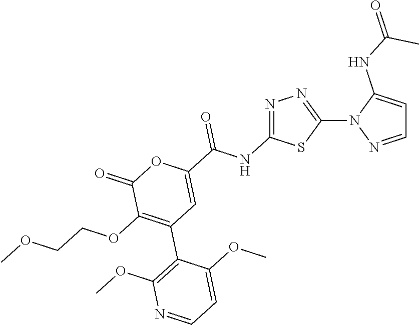 [SMILES: COCCOc1c(-c2c(OC)ccnc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2,4- |
| | | dimethoxypyridin-3-yl)-3-(2- |
| | | methoxyethoxy)-2-oxo-2H- |
| | | pyran-6-carboxamide |
| 20 | 16 | 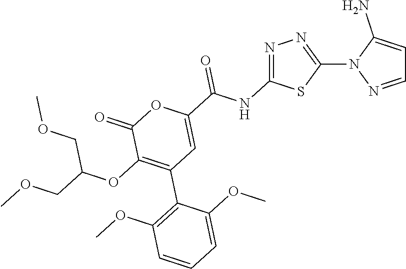 [SMILES: COCC(COC)Oc1c(-c2c(OC)cccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2,6- |
| | | dimethoxyphenyl)-3-((1,3- |
| | | dimethoxypropan-2-yl)oxy)-2- |
| | | oxo-2H-pyran-6-carboxamide |
| 20- Ac | 16 | 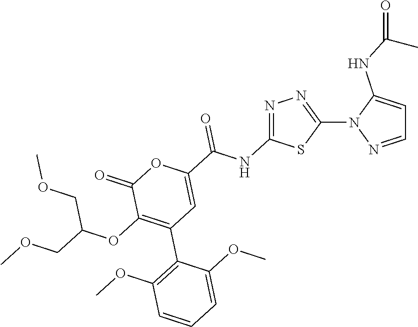 [SMILES: COCC(COC)Oc1c(-c2c(OC)cccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2,6- |
| | | dimethoxyphenyl)-3-((1,3- |
| | | dimethoxypropan-2-yl)oxy)-2- |
| | | oxo-2H-pyran-6-carboxamide |
| 21 | 17 | 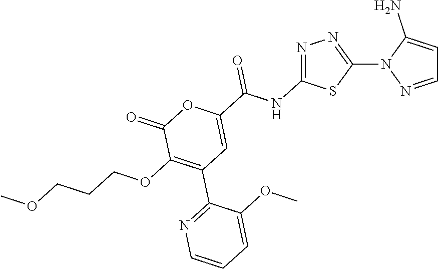 [SMILES: COCCCOc1c(-c2ncccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-3-(3- |
| | | methoxypropoxy)-4-(3- |
| | | methoxypyridin-2-yl)-2-oxo-2H- |
| | | pyran-6-carboxamide |
| 21- Ac | 17 | 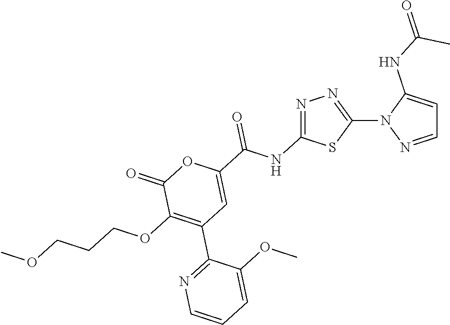 [SMILES: COCCCOc1c(-c2ncccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-3-(3- |
| | | methoxypropoxy)-4-(3- |
| | | methoxypyridin-2-yl)-2-oxo-2H- |
| | | pyran-6-carboxamide |
| 22 | 18 | 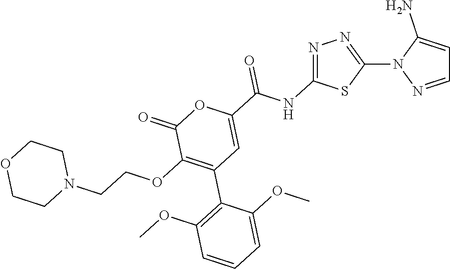 [SMILES: COc1cccc(OC)c1-c1cc(C(=O)Nc2nnc(-n3nccc3N)s2)oc(=O)c1OCCN1CCOCC1] |
| | | N-(5-(5-amino-1H-pyrazol-1-yl)- |
| | | 1,3,4-thiadiazol-2-yl)-4-(2,6- |
| | | dimethoxyphenyl)-3-(2- |
| | | morpholinoethoxy)-2-oxo-2H- |
| | | pyran-6-carboxamide |
| 22- Ac | 18 | 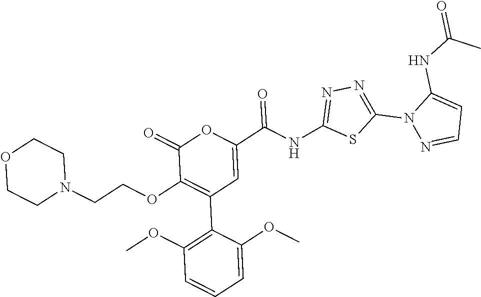 [SMILES: COc1cccc(OC)c1-c1cc(C(=O)Nc2nnc(-n3nccc3NC(C)=O)s2)oc(=O)c1OCCN1CCOCC1] |
| | | N-(5-(5-acetamido-1H-pyrazol-1- |
| | | yl)-1,3,4-thiadiazol-2-yl)-4-(2,6- |
| | | dimethoxyphenyl)-3-(2- |
| | | morpholinoethoxy)-2-oxo-2H- |
| | | pyran-6-carboxamide |
| 23 | 19 | 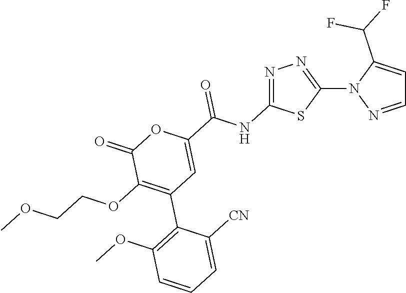 [SMILES: COCCOc1c(-c2c(C#N)cccc2OC)cc(C(=O)Nc2nnc(-n3nccc3C(F)F)s2)oc1=O] |
| | | 4-(2-cyano-6-methoxyphenyl)-N- |
| | | (5-(5-(difluoromethyl)-1H- |
| | | pyrazol-1-yl)-1,3,4-thiadiazol-2- |
| | | yl)-3-(2-methoxyethoxy)-2-oxo- |
| | | 2H-pyran-6-carboxamide |
| 24A* | 20 | 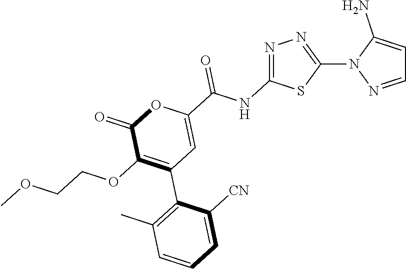 |
| | | (Ra)-N-(5-(5-amino-1H-pyrazol- |
| | | 1-yl)-1,3,4-thiadiazol-2-yl)-4-(2- |
| | | cyano-6-methylphenyl)-3-(2- |
| | | methoxyethoxy)-2-oxo-2H- |
| | | pyran-6-carboxamide |
| 24B* | 20 | 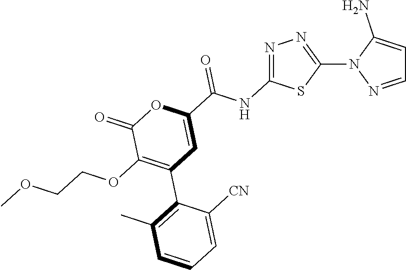 |
| | | (Sa)-N-(5-(5-amino-1H-pyrazol- |
| | | 1-yl)-1,3,4-thiadiazol-2-yl)-4-(2- |
| | | cyano-6-methylphenyl)-3-(2- |
| | | methoxyethoxy)-2-oxo-2H- |
| | | pyran-6-carboxamide |
| 24- Ac | 20 | 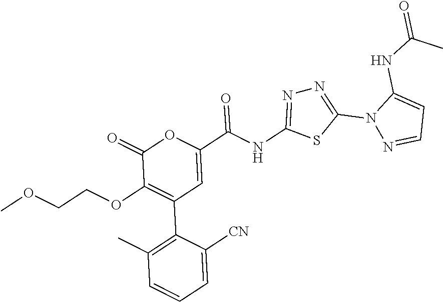 [SMILES: COCCOc1c(-c2c(C)cccc2C#N)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | cyano-6-methylphenyl)-3-(2- |
| | | methoxyethoxy)-2-oxo-2H- |
| | | pyran-6-carboxamide |
| 25 | 34 | 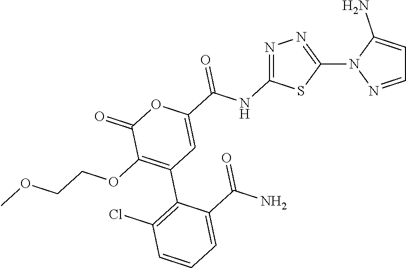 [SMILES: COCCOc1c(-c2c(Cl)cccc2C(N)=O)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2- |
| | | carbamoyl-6-chlorophenyl)-3-(2- |
| | | methoxyethoxy)-2-oxo-2H- |
| | | pyran-6-carboxamide |
| 25- Ac | 34 | 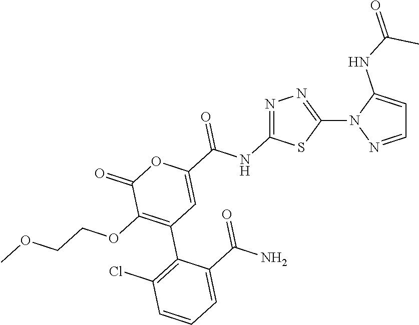 [SMILES: COCCOc1c(-c2c(Cl)cccc2C(N)=O)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | carbamoyl-6-chlorophenyl)-3-(2- |
| | | methoxyethoxy)-2-oxo-2H- |
| | | pyran-6-carboxamide |
| 26 | 21 | 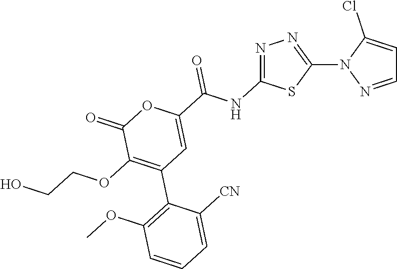 [SMILES: COc1cccc(C#N)c1-c1cc(C(=O)Nc2nnc(-n3nccc3Cl)s2)oc(=O)c1OCCO] |
| | | N-(5-(5-chloro-1H-pyrazol-1-yl)- |
| | | 1,3,4-thiadiazol-2-yl)-4-(2-cyano- |
| | | 6-methoxyphenyl)-3-(2- |
| | | hydroxyethoxy)-2-oxo-2H-pyran- |
| | | 6-carboxamide |
| 26- OtBu | 21 | 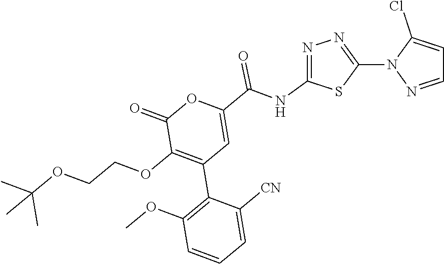 [SMILES: COc1cccc(C#N)c1-c1cc(C(=O)Nc2nnc(-n3nccc3Cl)s2)oc(=O)c1OCCOC(C)(C)C] |
| | | 3-(2-(tert-butoxy)ethoxy)-N-(5- |
| | | (5-chloro-1H-pyrazol-1-yl)-1,3,4- |
| | | thiadiazol-2-yl)-4-(2-cyano-6- |
| | | methoxyphenyl)-2-oxo-2H- |
| | | pyran-6-carboxamide |
| 27 | 22 | 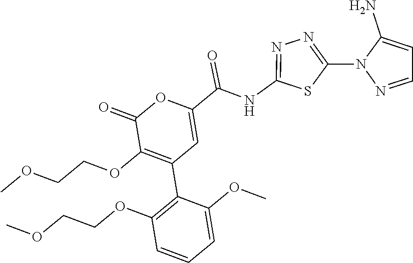 [SMILES: COCCOc1cccc(OC)c1-c1cc(C(=O)Nc2nnc(-n3nccc3N)s2)oc(=O)c1OCCOC] |
| | | N-(5-(5-amino-1H-pyrazol-1-yl)- |
| | | 1,3,4-thiadiazol-2-yl)-4-(2- |
| | | methoxy-6-(2- |
| | | methoxyethoxy)phenyl)-3-(2- |
| | | methoxyethoxy)-2-oxo-2H- |
| | | pyran-6-carboxamide |
| 27- Ac | 22 | 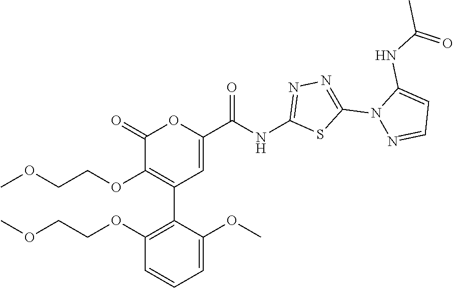 [SMILES: COCCOc1cccc(OC)c1-c1cc(C(=O)Nc2nnc(-n3nccc3NC(C)=O)s2)oc(=O)c1OCCOC] |
| | | N-(5-(5-acetamido-1H-pyrazol-1- |
| | | yl)-1,3,4-thiadiazol-2-yl)-4-(2- |
| | | methoxy-6-(2- |
| | | methoxyethoxy)phenyl)-3-(2- |
| | | methoxyethoxy)-2-oxo-2H- |
| | | pyran-6-carboxamide |
| 28 | 23 | 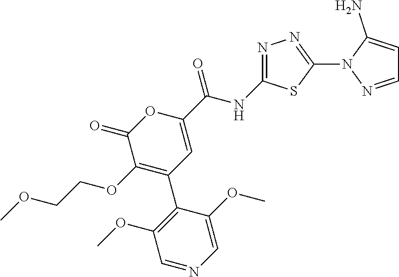 [SMILES: COCCOc1c(-c2c(OC)cncc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(3,5- |
| | | dimethoxypyridin-4-yl)-3-(2- |
| | | methoxyethoxy)-2-oxo-2H- |
| | | pyran-6-carboxamide |
| 28- Ac | 23 | 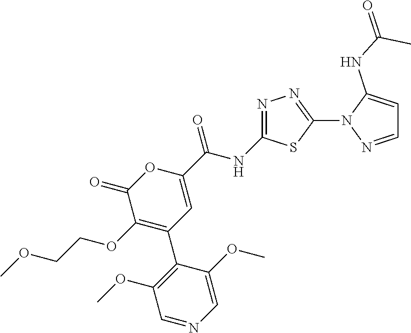 [SMILES: COCCOc1c(-c2c(OC)cncc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(3,5- |
| | | dimethoxypyridin-4-yl)-3-(2- |
| | | methoxyethoxy)-2-oxo-2H- |
| | | pyran-6-carboxamide |
| 29 | 24 | 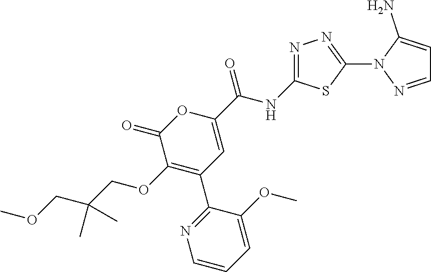 [SMILES: COCC(C)(C)COc1c(-c2ncccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-3-(3- |
| | | methoxy-2,2-dimethylpropoxy)- |
| | | 4-(3-methoxypyridin-2-yl)-2- |
| | | oxo-2H-pyran-6-carboxamide |
| 29- Ac | 24 | 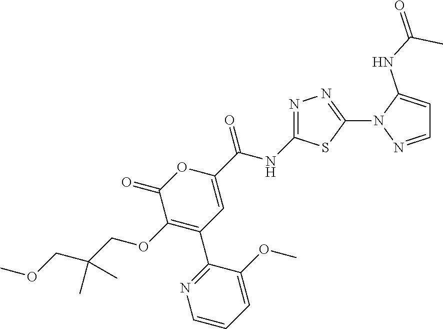 [SMILES: COCC(C)(C)COc1c(-c2ncccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-3-(3- |
| | | methoxy-2,2-dimethylpropoxy)- |
| | | 4-(3-methoxypyridin-2-yl)-2- |
| | | oxo-2H-pyran-6-carboxamide |
| 30 | TA | 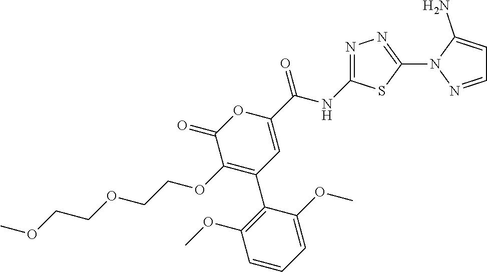 [SMILES: COCCOCCOc1c(-c2c(OC)cccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2,6- |
| | | dimethoxyphenyl)-3-(2-(2- |
| | | methoxyethoxy)ethoxy)-2-oxo- |
| | | 2H-pyran-6-carboxamide |
| 31 | 25 | 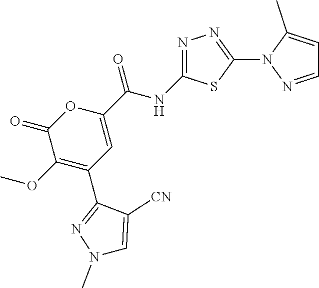 [SMILES: COc1c(-c2nn(C)cc2C#N)cc(C(=O)Nc2nnc(-n3nccc3C)s2)oc1=O] |
| | | 4-(4-cyano-1-methyl-1H-pyrazol- |
| | | 3-yl)-3-methoxy-N-(5-(5-methyl- |
| | | 1H-pyrazol-1-yl)-1,3,4- |
| | | thiadiazol-2-yl)-2-oxo-2H-pyran- |
| | | 6-carboxamide |
| 16 | 25 | 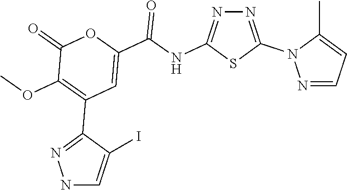 [SMILES: COc1c(-c2n[nH]cc2I)cc(C(=O)Nc2nnc(-n3nccc3C)s2)oc1=O] |
| | | 4-(4-iodo-1-methyl-1H-pyrazol- |
| | | 3-yl)-3-methoxy-N-(5-(5-methyl- |
| | | 1H-pyrazol-1-yl)-1,3,4- |
| | | thiadiazol-2-yl)-2-oxo-2H-pyran- |
| | | 6-carboxamide |
| 32 | 26 | 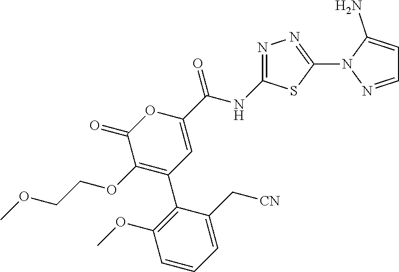 [SMILES: COCCOc1c(-c2c(CC#N)cccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2- |
| | | (cyanomethyl)-6- |
| | | methoxyphenyl)-3-(2- |
| | | methoxyethoxy)-2-oxo-2H- |
| | | pyran-6-carboxamide |
| 32- Ac | 26 | 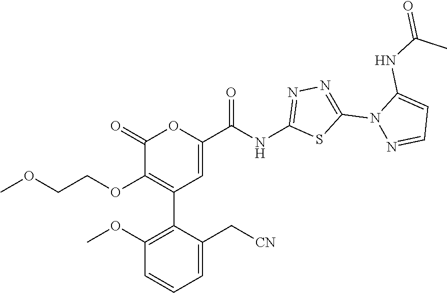 [SMILES: COCCOc1c(-c2c(CC#N)cccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | (cyanomethyl)-6- |
| | | methoxyphenyl)-3-(2- |
| | | methoxyethoxy)-2-oxo-2H- |
| | | pyran-6-carboxamide |
| 33 | 27 | 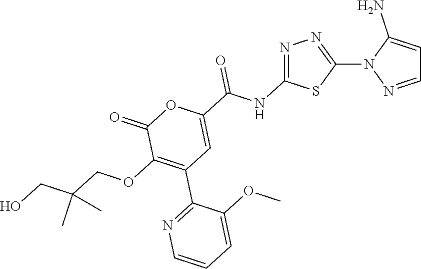 [SMILES: COc1cccnc1-c1cc(C(=O)Nc2nnc(-n3nccc3N)s2)oc(=O)c1OCC(C)(C)CO] |
| | | N-(5-(5-amino-1H-pyrazol-1-yl)- |
| | | 1,3,4-thiadiazol-2-yl)-3-(3- |
| | | hydroxy-2,2-dimethylpropoxy)- |
| | | 4-(3-methoxypyridin-2-yl)-2- |
| | | oxo-2H-pyran-6-carboxamide |
| 34A* | 28 | 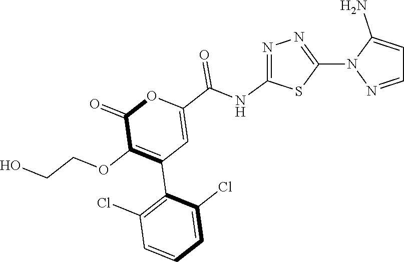 |
| | | (Sa)-N-(5-(5-amino-1H-pyrazol- |
| | | 1-yl)-1,3,4-thiadiazol-2-yl)-4-(2- |
| | | chloro-6-methoxyphenyl)-3-(2- |
| | | hydroxyethoxy)-2-oxo-2H-pyran- |
| | | 6-carboxamide |
| 34 B* | 28 | 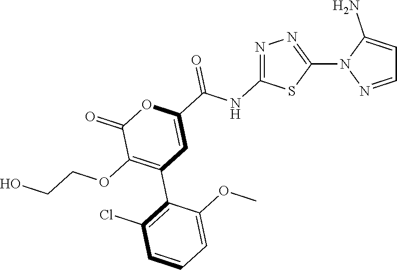 |
| | | (Ra)-N-(5-(5-amino-1H-pyrazol- |
| | | 1-yl)-1,3,4-thiadiazol-2-yl)-4-(2- |
| | | chloro-6-methoxyphenyl)-3-(2- |
| | | hydroxyethoxy)-2-oxo-2H-pyran- |
| | | 6-carboxamide |
| 34- Ac- OtBu | 28 | 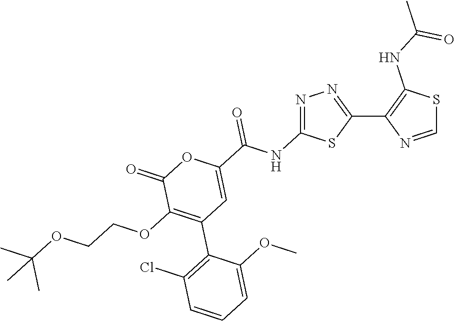 [SMILES: COc1cccc(Cl)c1-c1cc(C(=O)Nc2nnc(-c3ncsc3NC(C)=O)s2)oc(=O)c1OCCOC(C)(C)C] |
| | | N-(5-(5-acetamido-1H-pyrazol-1- |
| | | yl)-1,3,4-thiadiazol-2-yl)-3-(2- |
| | | (tert-butoxy)ethoxy)-4-(2-chloro- |
| | | 6-methoxphenyl)-2-oxo-2H- |
| | | pyran-6-carboxamide |
| 35 | 44 | 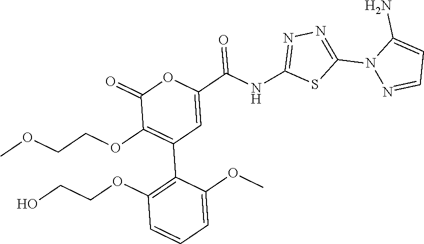 [SMILES: COCCOc1c(-c2c(OC)cccc2OCCO)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2-(2- |
| | | hydroxyethoxy)-6- |
| | | methoxyphenyl)-3-(2- |
| | | methoxyethoxy)-2-oxo-2H- |
| | | pyran-6-carboxamide |
| 35- Ac | 44 | 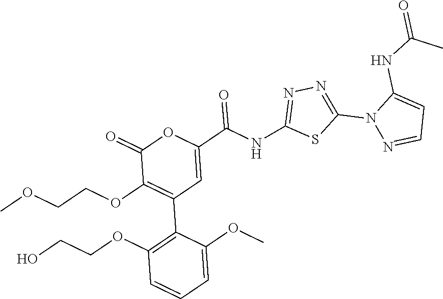 [SMILES: COCCOc1c(-c2c(OC)cccc2OCCO)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2-(2- |
| | | hydroxyethoxy)-6- |
| | | methoxyphenyl)-3-(2- |
| | | methoxyethoxy)-2-oxo-2H- |
| | | pyran-6-carboxamide |
| 36 | 29 | 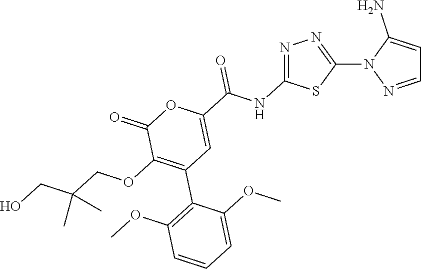 [SMILES: COc1cccc(OC)c1-c1cc(C(=O)Nc2nnc(-n3nccc3N)s2)oc(=O)c1OCC(C)(C)CO] |
| | | N-(5-(5-amino-1H-pyrazol-1-yl)- |
| | | 1,3,4-thiadiazol-2-yl)-4-(2,6- |
| | | dimethoxyphenyl)-3-(3-hydroxy- |
| | | 2,2-dimethylpropoxy)-2-oxo-2H- |
| | | pyran-6-carboxamide |
| 37A* | 30 | 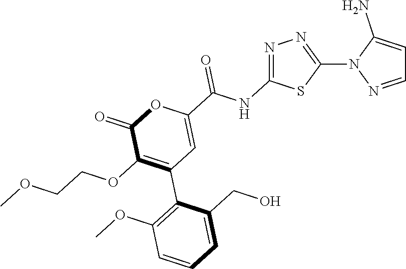 |
| | | (Sa)-N-(5-(5-amino-1H-pyrazol- |
| | | 1-yl)-1,3,4-thiadiazol-2-yl)-4-(2- |
| | | chloro-6-methoxyphenyl)-3-(2- |
| | | hydroxyethoxy)-2-oxo-2H-pyran- |
| | | 6-carboxamide |
| 37B* | 30 | 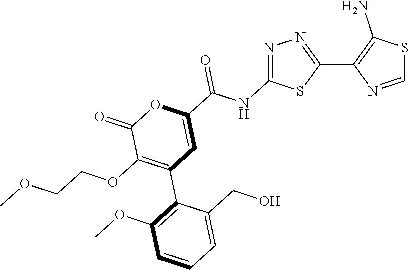 |
| | | (Ra)-N-(5-(5-amino-1H-pyrazol- |
| | | 1-yl)-1,3,4-thiadiazol-2-yl)-4-(2- |
| | | chloro-6-methoxyphenyl)-3-(2- |
| | | hydroxyethoxy)-2-oxo-2H-pyran- |
| | | 6-carboxamide |
| 37- Ac | 26 | 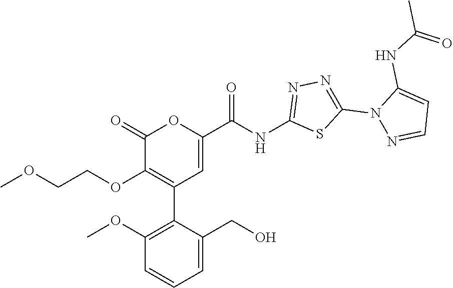 [SMILES: COCCOc1c(-c2c(CO)cccc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | (hydroxymethyl)-6- |
| | | methoxyphenyl)-3-(2- |
| | | methoxyethoxy)-2-oxo-2H- |
| | | pyran-6-carboxamide |
| 38A* | TA | 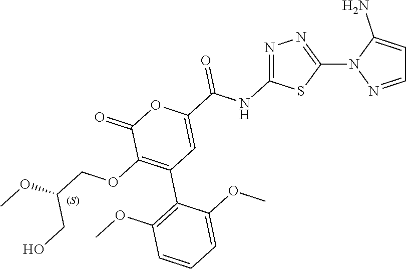 |
| | | (S)-N-(5-(5-amino-1H-pyrazol-1- |
| | | yl)-1,3,4-thiadiazol-2-yl)-4-(2,6- |
| | | dimethoxyphenyl)-3-(3-hydroxy- |
| | | 2-methoxypropoxy)-2-oxo-2H- |
| | | pyran-6-carboxamide |
| 38B* | TA | 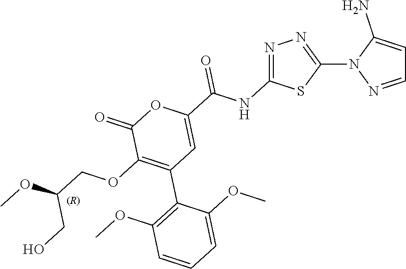 |
| | | (R)-N-(5-(5-amino-1H-pyrazol-1- |
| | | yl)-1,3,4-thiadiazol-2-yl)-4-(2,6- |
| | | dimethoxyphenyl)-3-(3-hydroxy- |
| | | 2-methoxypropoxy)-2-oxo-2H- |
| | | pyran-6-carboxamide |
| 39 | 31 | 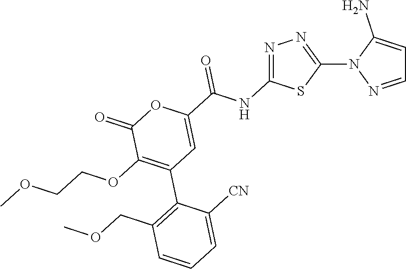 [SMILES: COCCOc1c(-c2c(C#N)cccc2COC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2-cyano- |
| | | 6-(methoxymethyl)phenyl)-3-(2- |
| | | methoxyethoxy)-2-oxo-2H- |
| | | pyran-6-carboxamide |
| 39- Ac | 31 | 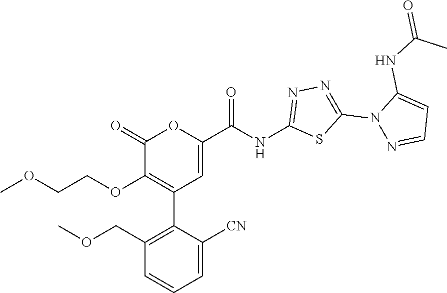 [SMILES: COCCOc1c(-c2c(C#N)cccc2COC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | cyano-6- |
| | | (methoxymethyl)phenyl)-3-(2- |
| | | methoxyethoxy)-2-oxo-2H- |
| | | pyran-6-carboxamide |
| 40 | 32 | 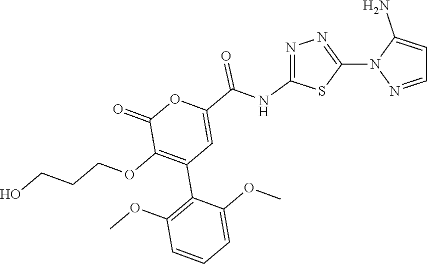 [SMILES: COc1cccc(OC)c1-c1cc(C(=O)Nc2nnc(-n3nccc3N)s2)oc(=O)c1OCCCO] |
| | | N-(5-(5-amino-1H-pyrazol-1-yl)- |
| | | 1,3,4-thiadiazol-2-yl)-4-(2,6- |
| | | dimethoxyphenyl)-3-(3- |
| | | hydroxypropoxy)-2-oxo-2H- |
| | | pyran-6-carboxamide |
| 40- Ac | 32 | 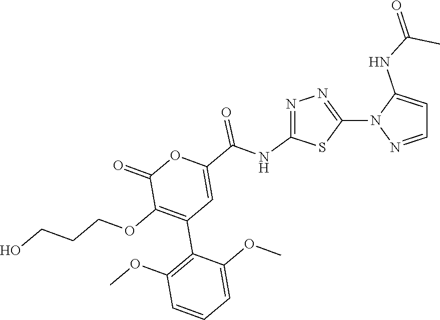 [SMILES: COc1cccc(OC)c1-c1cc(C(=O)Nc2nnc(-n3nccc3NC(C)=O)s2)oc(=O)c1OCCCO] |
| | | N-(5-(5-acetamido-1H-pyrazol-1- |
| | | yl)-1,3,4-thiadiazol-2-yl)-4-(2,6- |
| | | dimethoxyphenyl)-3-(3- |
| | | hydroxypropoxy)-2-oxo-2H- |
| | | pyran-6-carboxamide |
| 41 | TA | 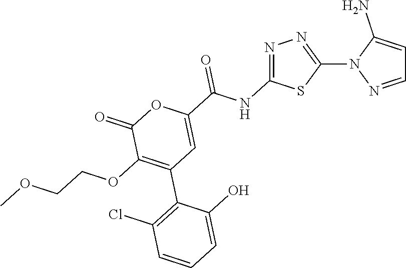 [SMILES: COCCOc1c(-c2c(O)cccc2Cl)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2- |
| | | chloro-6-hydroxyphenyl)-3-(2- |
| | | methoxyethoxy)-2-oxo-2H- |
| | | pyran-6-carboxamide |
| 42 | TA | 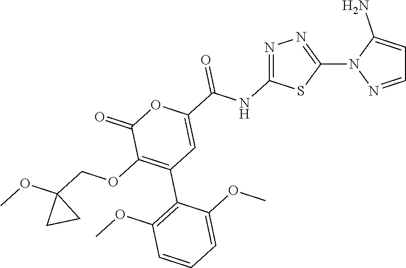 [SMILES: COc1cccc(OC)c1-c1cc(C(=O)Nc2nnc(-n3nccc3N)s2)oc(=O)c1OCC1(OC)CC1] |
| | | N-(5-(5-amino-1H-pyrazol-1-yl)- |
| | | 1,3,4-thiadiazol-2-yl)-4-(2,6- |
| | | dimethoxyphenyl)-3-((1- |
| | | methoxycyclopropoyl)methoxy)-2- |
| | | oxo-2H-pyran-6-carboxamide |
| 43A* | 33 | 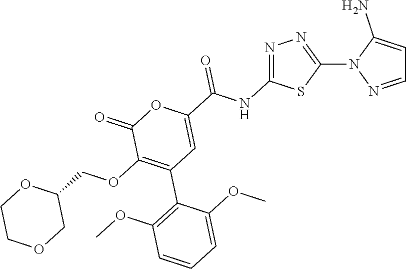 [SMILES: COc1cccc(OC)c1-c1cc(C(=O)Nc2nnc(-n3nccc3N)s2)oc(=O)c1OC[C@@H]1COCCO1] |
| | | (S)-3-((1,4-dioxan-2- |
| | | yl)methoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 43B* | 33 | 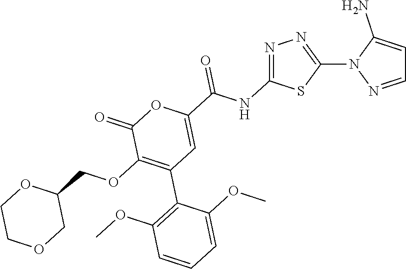 [SMILES: COc1cccc(OC)c1-c1cc(C(=O)Nc2nnc(-n3nccc3N)s2)oc(=O)c1OC[C@H]1COCCO1] |
| | | (R)-3-((1,4-dioxan-2- |
| | | yl)methoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 43- Ac | 33 | 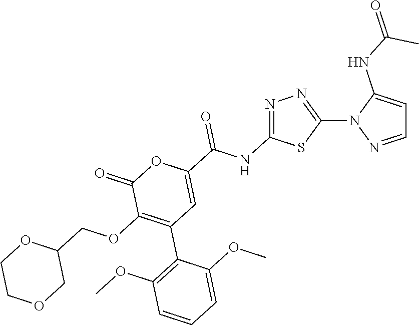 [SMILES: COc1cccc(OC)c1-c1cc(C(=O)Nc2nnc(-n3nccc3NC(C)=O)s2)oc(=O)c1OCC1COCCO1] |
| | | 3-((1,4-dioxan-2-yl)methoxy)-N- |
| | | (5-(5-acetamido-1H-pyrazol-1- |
| | | yl)-1,3,4-thiadiazol-2-yl)-4-(2,6- |
| | | dimethoxyphenyl)-2-oxo-2H- |
| | | pyran-6-carboxamide |
| 44 | TA | 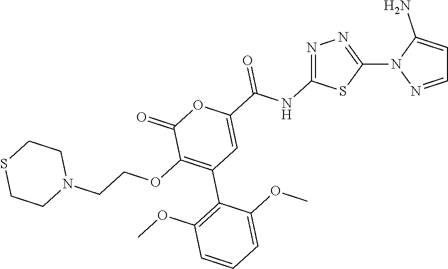 [SMILES: COc1cccc(OC)c1-c1cc(C(=O)Nc2nnc(-n3nccc3N)s2)oc(=O)c1OCCN1CCSCC1] |
| | | N-(5-(5-amino-1H-pyrazol-1-yl)- |
| | | 1,3,4-thiadiazol-2-yl)-4-(2,6- |
| | | dimethoxyphenyl)-2-oxo-3-(2- |
| | | thiomorpholinoethoxy)-2H- |
| | | pyran-6-carboxamide |
| 45 | 34 | + 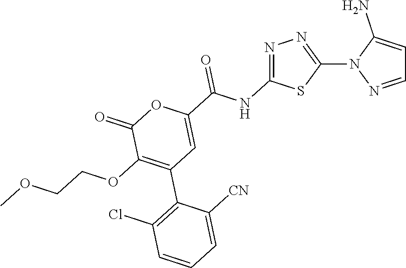 [SMILES: COCCOc1c(-c2c(Cl)cccc2C#N)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2- |
| | | chloro-6-cyanophenyl)-3-(2- |
| | | methoxyethoxy)-2-oxo-2H- |
| | | pyran-6-carboxamide |
| 45- Ac | 34 | 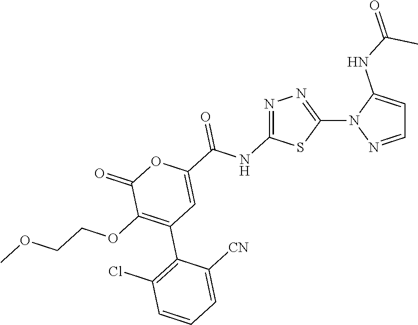 [SMILES: COCCOc1c(-c2c(Cl)cccc2C#N)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | chloro-6-cyanophenyl)-3-(2- |
| | | methoxyethoxy)-2-oxo-2H- |
| | | pyran-6-carboxamide |
| 46A | 35 | 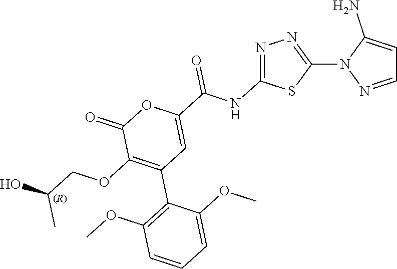 |
| | | (R)-N-(5-(5-amino-1H-pyrazol-1- |
| | | yl)-1,3,4-thiadiazol-2-yl)-4-(2,6- |
| | | dimethoxyphenyl)-3-(2- |
| | | hydroxypropoxy)-2-oxo-2H- |
| | | pyran-6-carboxamide |
| 46B | 35 | 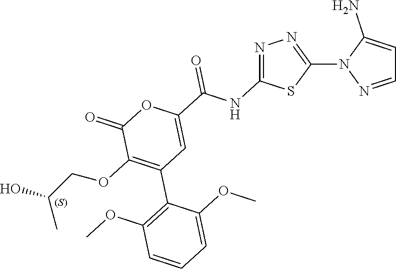 |
| | | (S)-N-(5-(5-amino-1H-pyrazol-1- |
| | | yl)-1,3,4-thiadiazol-2-yl)-4-(2,6- |
| | | dimethoxyphenyl)-3-(2- |
| | | hydroxypropoxy)-2-oxo-2H- |
| | | pyran-6-carboxamide |
| 47A | 36 | 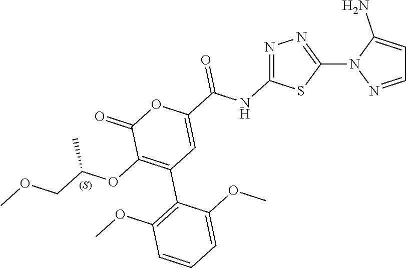 |
| | | (S)-N-(5-(5-amino-1H-pyrazol-1- |
| | | yl)-1,3,4-thiadiazol-2-yl)-4-(2,6- |
| | | dimethoxyphenyl)-3-((1- |
| | | methoxypropan-2-yl)oxy)-2-oxo- |
| | | 2H-pyran-6-carboxamide |
| 47A- Ac | 36 | 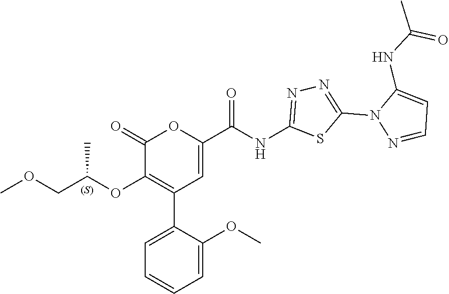 |
| | | (S)-N-(5-(5-acetamido-1H- |
| | | pyrazol-1-yl)-1,3,4-thiadiazol-2- |
| | | yl)-4-(2,6-dimethoxyphenyl)-3- |
| | | ((1-methoxypropan-2-yl)oxy)-2- |
| | | oxo-2H-pyran-6-carboxamide |
| 47B | 36 | 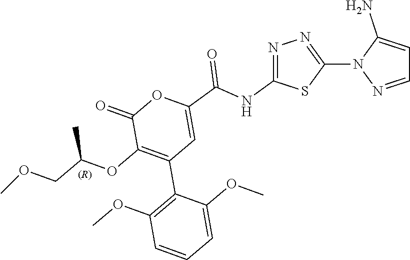 |
| | | (R)-N-(5-(5-amino-1H-pyrazol-1- |
| | | yl)-1,3,4-thiadiazol-2-yl)-4-(2,6- |
| | | dimethoxyphenyl)-3-((1- |
| | | methoxypropan-2-yl)oxy)-2-oxo- |
| | | 2H-pyran-6-carboxamide |
| 47B- Ac | 36 | 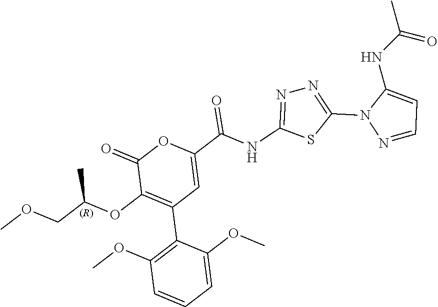 |
| | | (R)-N-(5-(5-acetamido-1H- |
| | | pyrazol-1-yl)-1,3,4-thiadiazol-2- |
| | | yl)-4-(2,6-dimethoxyphenyl)-3- |
| | | ((1-methoxypropan-2-yl)oxy)-2- |
| | | oxo-2H-pyran-6-carboxamide |
| 48 | 37 | 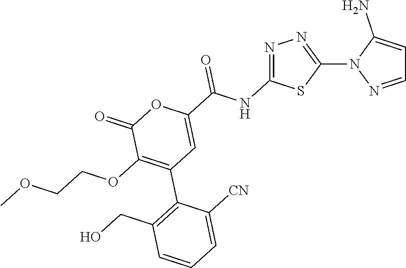 [SMILES: COCCOc1c(-c2c(C#N)cccc2CO)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2-cyano- |
| | | 6-(hydroxymethyl)phenyl)-3-(2- |
| | | methoxyethoxy)-2-oxo-2H- |
| | | pyran-6-carboxamide |
| 49 | TA | 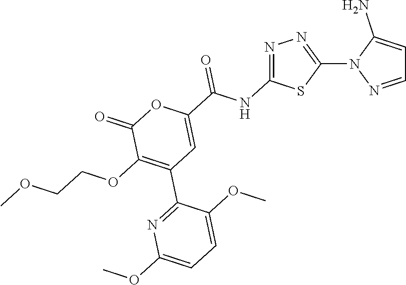 [SMILES: COCCOc1c(-c2nc(OC)ccc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(3,6- |
| | | dimethoxypyridin-2-yl)-3-(2- |
| | | methoxyethoxy)-2-oxo-2H- |
| | | pyran-6-carboxamide |
| 50 | 38 | 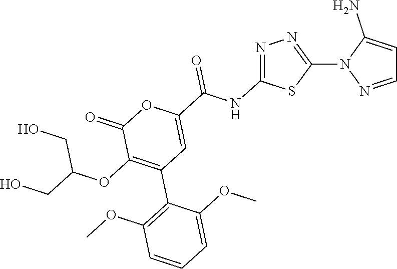 [SMILES: COc1cccc(OC)c1-c1cc(C(=O)Nc2nnc(-n3nccc3N)s2)oc(=O)c1OC(CO)CO] |
| | | N-(5-(5-amino-1H-pyrazol-1-yl)- |
| | | 1,3,4-thiadiazol-2-yl)-3-((1,3- |
| | | dihdyroxypropan-2-yl)oxy)-4- |
| | | (2,6-dimethoxyphenyl)-2-oxo- |
| | | 2H-pyran-6-carboxamide |
| 51 | 39 | 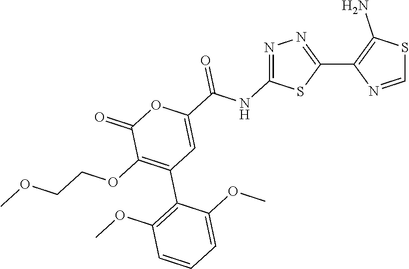 [SMILES: COCCOc1c(-c2c(OC)cccc2OC)cc(C(=O)Nc2nnc(-c3ncsc3N)s2)oc1=O] |
| | | N-(5-(5-aminothiazol-4-yl)-1,3,4- |
| | | thiadiazol-2-yl)-4-(2,6- |
| | | dimethoxyphenyl)-3-(2- |
| | | methoxyethoxy)-2-oxo-2H- |
| | | pyran-6-carboxamide |
| 51- Ac | 39 | 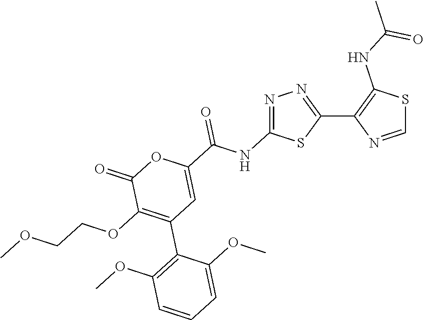 [SMILES: COCCOc1c(-c2c(OC)cccc2OC)cc(C(=O)Nc2nnc(-c3ncsc3NC(C)=O)s2)oc1=O] |
| | | N-(5-(5-acetamidothiazol-4-yl)- |
| | | 1,3,4-thiadiazol-2-yl)-4-(2,6- |
| | | dimethoxyphenyl)-3-(2- |
| | | methoxyethoxy)-2-oxo-2H- |
| | | pyran-6-carboxamide |
| 52A* | TA | 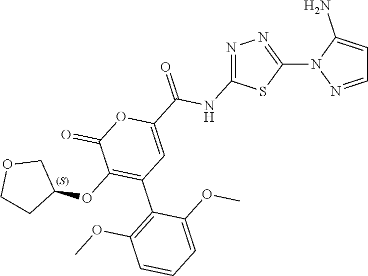 |
| | | (S)-N-(5-(5-amino-1H-pyrazol-1- |
| | | yl)-1,3,4-thiadiazol-2-yl)-4-(2,6- |
| | | dimethoxyphenyl)-2-oxo-3- |
| | | ((tetrahydrofuran-3-yl)oxy)-2H- |
| | | pyran-6-carboxamide |
| 52B* | TA | 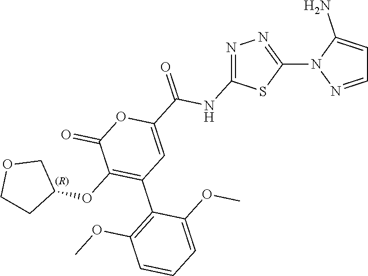 |
| | | (R)-N-(5-(5-amino-1H-pyrazol-1- |
| | | yl)-1,3,4-thiadiazol-2-yl)-4-(2,6- |
| | | dimethoxyphenyl)-2-oxo-3- |
| | | ((tetrahydrofuran-3-yl)oxy)-2H- |
| | | pyran-6-carboxamide |
| 53 | TA | 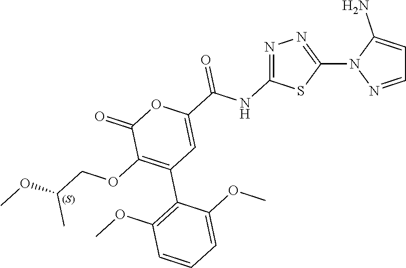 |
| | | (S)-N-(5-(5-amino-1H-pyrazol-1- |
| | | yl)-1,3,4-thiadiazol-2-yl)-4-(2,6- |
| | | dimethoxyphenyl)-3-(2- |
| | | methoxypropoxy)-2-oxo-2H- |
| | | pyran-6-carboxamide |
| 54 | TA | 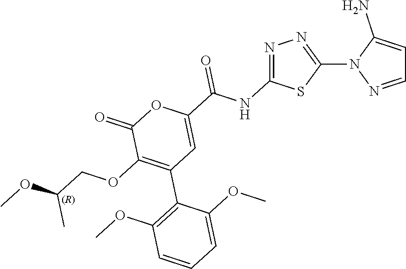 |
| | | (R)-N-(5-(5-amino-1H-pyrazol-1- |
| | | yl)-1,3,4-thiadiazol-2-yl)-4-(2,6- |
| | | dimethoxyphenyl)-3-(2- |
| | | methoxypropoxy)-2-oxo-2H- |
| | | pyran-6-carboxamide |
| 55A* | 40 | 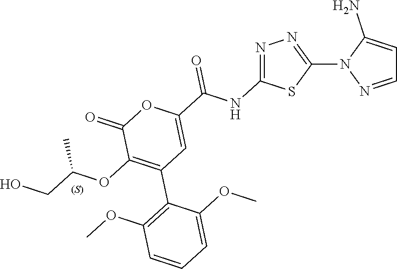 |
| | | (S)-N-(5-(5-amino-1H-pyrazol-1- |
| | | yl)-1,3,4-thiadiazol-2-yl)-4-(2,6- |
| | | dimethoxyphenyl)-3-((1- |
| | | hydroxypropan-2-yl)oxy)-2-oxo- |
| | | 2H-pyran-6-carboxamide |
| 55B* | 40 | 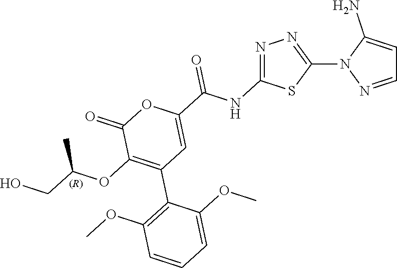 |
| | | (R)-N-(5-(5-amino-1H-pyrazol-1- |
| | | yl)-1,3,4-thiadiazol-2-yl)-4-(2,6- |
| | | dimethoxyphenyl)-3-((1- |
| | | hydroxypropan-2-yl)oxy)-2-oxo- |
| | | 2H-pyran-6-carboxamide |
| 56A* | TA | 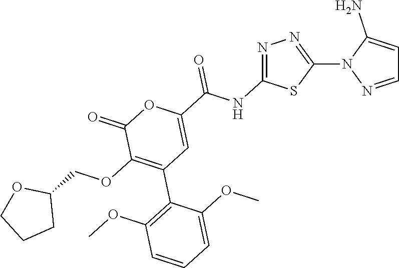 [SMILES: COc1cccc(OC)c1-c1cc(C(=O)Nc2nnc(-n3nccc3N)s2)oc(=O)c1OC[C@@H]1CCCO1] |
| | | (S)-N-(5-(5-amino-1H-pyrazol-1- |
| | | yl)-1,3,4-thiadiazol-2-yl)-4-(2,6- |
| | | dimethoxyphenyl)-2-oxo-3- |
| | | ((tetrahydrofuran-2-yl)methoxy)- |
| | | 2H-pyran-6-carboxamide |
| 56B* | TA | 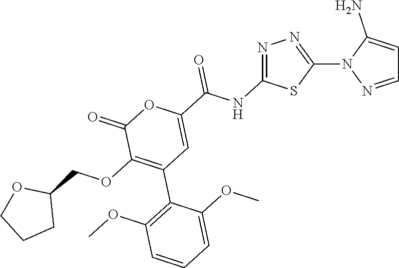 [SMILES: COc1cccc(OC)c1-c1cc(C(=O)Nc2nnc(-n3nccc3N)s2)oc(=O)c1OC[C@H]1CCCO1] |
| | | (R)-N-(5-(5-amino-1H-pyrazol-1- |
| | | yl)-1,3,4-thiadiazol-2-yl)-4-(2,6- |
| | | dimethoxyphenyl)-2-oxo-3- |
| | | ((tetrahydrofuran-2-yl)methoxy)- |
| | | 2H-pyran-6-carboxamide |
| 57A | TA | 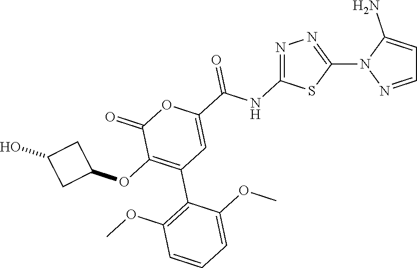 |
| | | N-(5-(5-amino-1H-pyrazol-1-yl)- |
| | | 1,3,4-thiadiazol-2-yl)-4-(2,6- |
| | | dimethoxyphenyl)-3-((1,3-trans)-3- |
| | | hydroxycyclobutoxy)-2-oxo-2H- |
| | | pyran-6-carboxamide |
| 57B | TA | 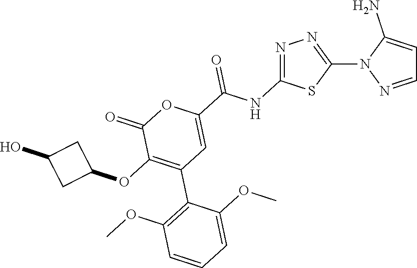 |
| | | N-(5-(5-amino-1H-pyrazol-1-yl)- |
| | | 1,3,4-thiadiazol-2-yl)-4-(2,6- |
| | | dimethoxyphenyl)-3-((1,3-cis)-3- |
| | | hydroyxcyclobutoxy)-2-oxo-2H- |
| | | pyran-6-carboxamide |
| 58 | TA | 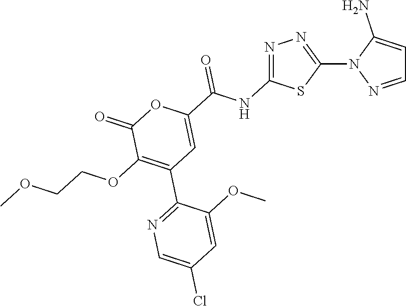 [SMILES: COCCOc1c(-c2ncc(Cl)cc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(5-chloro- |
| | | 3-methoxypyridin-2-yl)-3-(2- |
| | | methoxyethoxy)-2-oxo-2H-pyran- |
| | | 6-carboxamide |
| 59 | 41 | 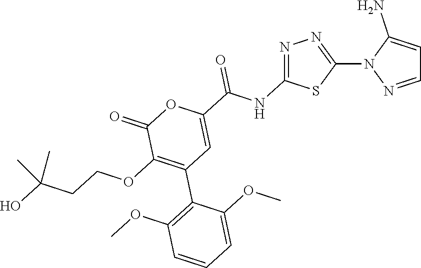 [SMILES: COc1cccc(OC)c1-c1cc(C(=O)Nc2nnc(-n3nccc3N)s2)oc(=O)c1OCCC(C)(C)O] |
| | | N-(5-(5-amino-1H-pyrazol-1-yl)- |
| | | 1,3,4-thiadiazol-2-yl)-4-(2,6- |
| | | dimethoxyphenyl)-3-(3-hydroxy-3- |
| | | methylbutoxy)-2-oxo-2H-pyran-6- |
| | | carboxamide |
| 59- Ac | 41 | 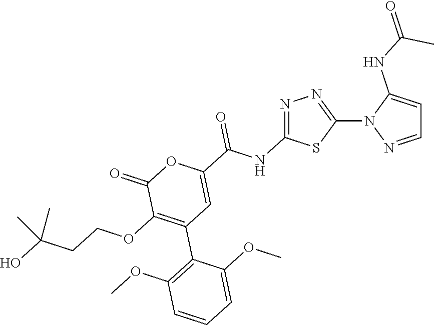 [SMILES: COc1cccc(OC)c1-c1cc(C(=O)Nc2nnc(-n3nccc3NC(C)=O)s2)oc(=O)c1OCCC(C)(C)O] |
| | | N-(5-(5-acetamido-1H-pyrazol-1- |
| | | yl)-1,3,4-thiadiazol-2-yl)-4-(2,6- |
| | | dimethoxyphenyl)-3-(3-hydroxy-3- |
| | | methylbutoxy)-2-oxo-2H-pyran-6- |
| | | carboxamide |
| 60 | 42 | 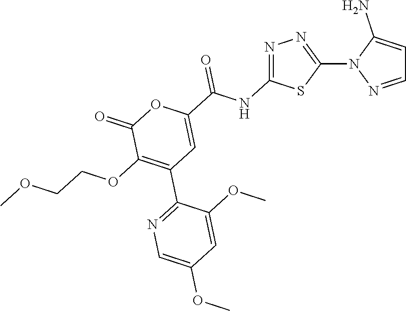 [SMILES: COCCOc1c(-c2ncc(OC)cc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(3,5- |
| | | dimethoxypyridin-2-yl)-3-(2- |
| | | methoxyethoxy)-2-oxo-2H-pyran- |
| | | 6-carboxamide |
| 60- Ac | 42 | 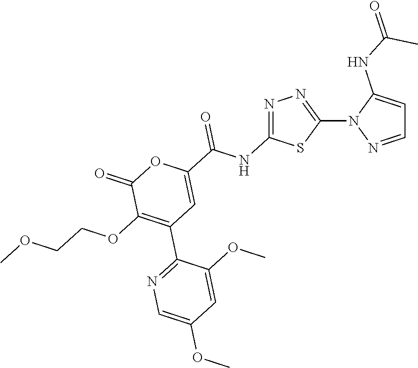 [SMILES: COCCOc1c(-c2ncc(OC)cc2OC)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(3,5- |
| | | dimethoxypyridin-2-yl)-3-(2- |
| | | methoxyethoxy)-2-oxo-2H-pyran- |
| | | 6-carboxamide |
| 61 | TA | 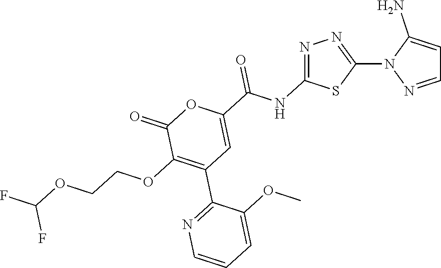 [SMILES: COc1cccnc1-c1cc(C(=O)Nc2nnc(-n3nccc3N)s2)oc(=O)c1OCCOC(F)F] |
| | | N-(5-(5-amino-1H-pyrazol-1-yl)- |
| | | 1,3,4-thiadiazol-2-yl)-3-(2- |
| | | (difluoromethoxy)ethoxy)-4-(3- |
| | | methoxypyridin-2-yl)-2-oxo-2H- |
| | | pyran-6-carboxamide |
| 62A* | 43 | 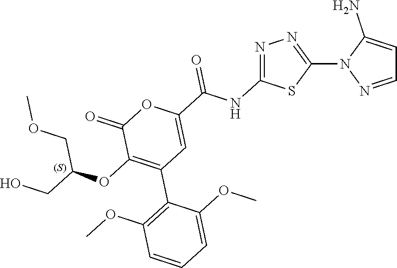 |
| | | (S)-N-(5-(5-amino-1H-pyrazol-1- |
| | | yl)-1,3,4-thiadiazol-2-yl)-4-(2,6- |
| | | dimethoxyphenyl)-3-((1-hydroxy- |
| | | 3-methoxypropan-2-yl)oxy)-2-oxo- |
| | | 2H-pyran-6-carboxamide |
| 62B* | 43 | 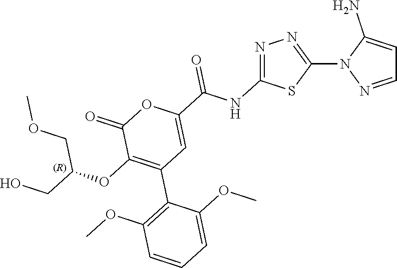 |
| | | (R)-N-(5-(5-amino-1H-pyrazol-1- |
| | | yl)-1,3,4-thiadiazol-2-yl)-4-(2,6- |
| | | dimethoxyphenyl)-3-((1-hydroxy- |
| | | 3-methoxypropan-2-yl)oxy)-2-oxo- |
| | | 2H-pyran-6-carboxamide |
| 63A* | 43 | 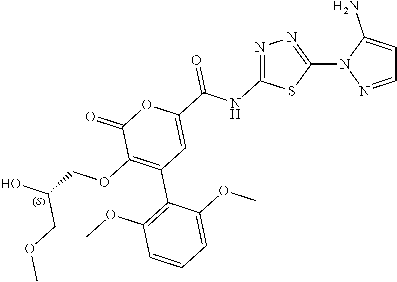 |
| | | (S)-N-(5-(5-amino-1H-pyrazol-1- |
| | | yl)-1,3,4-thiadiazol-2-yl)-4-(2,6- |
| | | dimethoxyphenyl)-3-(2-hydroxy-3- |
| | | methoxypropoxy)-2-oxo-2H-pyran- |
| | | 6-carboxamide |
| 63B* | 43 | 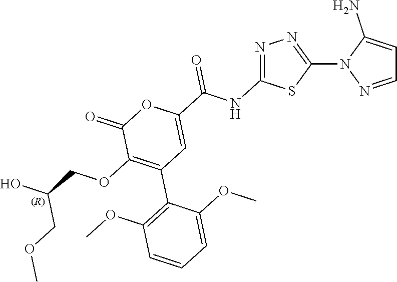 |
| | | (R)-N-(5-(5-amino-1H-pyrazol-1- |
| | | yl)-1,3,4-thiadiazol-2-yl)-4-(2,6- |
| | | dimethoxyphenyl)-3-(2-hydroxy-3- |
| | | methoxypropoxy)-2-oxo-2H-pyran- |
| | | 6-carboxamide |
| 64A* | TA | 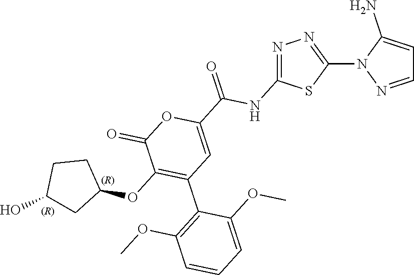 |
| | | N-(5-(5-amino-1H-pyrazol-1-yl)- |
| | | 1,3,4-thiadiazol-2-yl)-4-(2,6- |
| | | dimethoxyphenyl)-3-(((1R,3R)-3- |
| | | hydroxycyclopentyl)oxy)-2-oxo- |
| | | 2H-pyran-6-carboxamide |
| 64B* | TA | 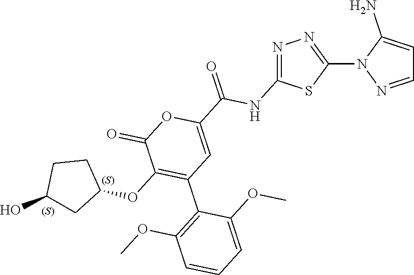 |
| | | N-(5-(5-amino-1H-pyrazol-1-yl)- |
| | | 1,3,4-thiadiaozl-2-yl)-4-(2,6- |
| | | dimethoxyphenyl)-3-(((1S,3S)-3- |
| | | hydroxycyclopentyl)oxy)-2-oxo- |
| | | 2H-pyran-6-carboxamide |
| 64C* | TA | 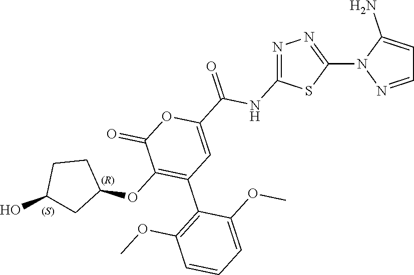 |
| | | N-(5-(5-amino-1H-pyrazol-1-yl)- |
| | | 1,3,4-thiadiaozl-2-yl)-4-(2,6- |
| | | dimethoxyphenyl)-3-(((1R,3S)-3- |
| | | hydroxycyclopentyl)oxy)-2-oxo- |
| | | 2H-pyran-6-carboxamide |
| 64D* | TA | 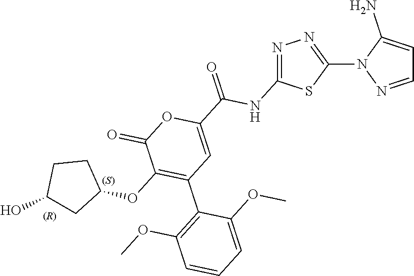 |
| | | N-(5-(5-amino-1H-pyrazol-1-yl)- |
| | | 1,3,4-thiadiazol-2-yl)-4-(2,6- |
| | | dimethoxyphenyl)-3-(((1S,3R)-3- |
| | | hydroxycyclopentyl)oxy)-2-oxo- |
| | | 2H-pyran-6-carboxamide |
| 65 | TA | 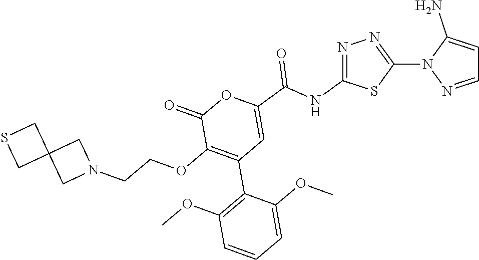 [SMILES: COc1cccc(OC)c1-c1cc(C(=O)Nc2nnc(-n3nccc3N)s2)oc(=O)c1OCCN1CC2(CSC2)C1] |
| | | 3-(2-(2-thia-6-azaspiro[3.3]heptan- |
| | | 6-yl)ethoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 66 | TA | 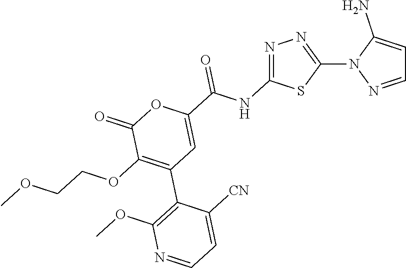 [SMILES: COCCOc1c(-c2c(C#N)ccnc2OC)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(4-cyano- |
| | | 2-methoxypyridin-3-yl)-3-(2- |
| | | methoxyethoxy)-2-oxo-2H-pyran- |
| | | 6-carboxamide |
| 67 | 44 | 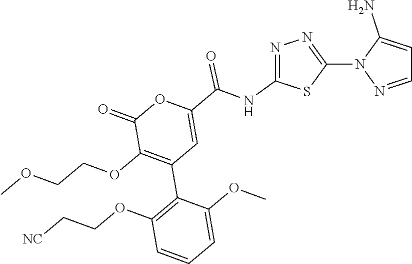 [SMILES: COCCOc1c(-c2c(OC)cccc2OCCC#N)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2-(2- |
| | | cyanoethoxy)-6-methoxyphenyl)-3- |
| | | (2-methoxyethoxy)-2-oxo-2H- |
| | | pyran-6-carboxamide |
| 67- Ac | 44 | 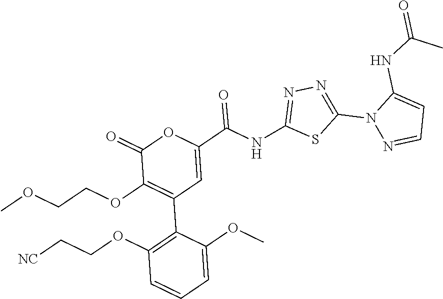 [SMILES: COCCOc1c(-c2c(OC)cccc2OCCC#N)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2-(2- |
| | | cyanoethoxy)-6-methoxyphenyl)-3- |
| | | (2-methoxyethoxy)-2-oxo-2H- |
| | | pyran-6-carboxamide |
| 68 | 45 | 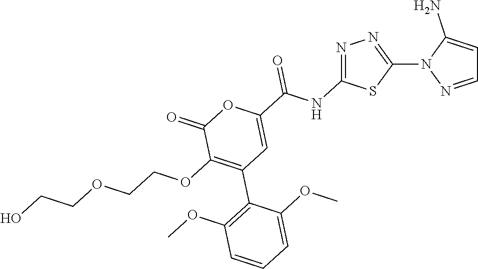 [SMILES: COc1cccc(OC)c1-c1cc(C(=O)Nc2nnc(-n3nccc3N)s2)oc(=O)c1OCCOCCO] |
| | | N-(5-(5-amino-1H-pyrazol-1-yl)- |
| | | 1,3,4-thiadiazol-2-yl)-4-(2,6- |
| | | dimethoxyphenyl)-3-(2-(2- |
| | | hydroxyethoxy)ethoxy)-2-oxo-2H- |
| | | pyran-6-carboxamide |
| 69 | TA | 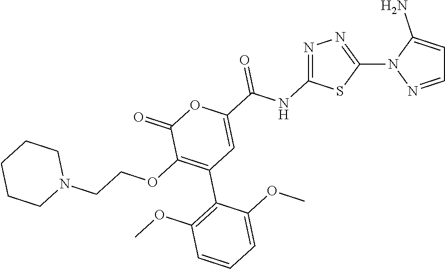 [SMILES: COc1cccc(OC)c1-c1cc(C(=O)Nc2nnc(-n3nccc3N)s2)oc(=O)c1OCCN1CCCCC1] |
| | | N-(5-(5-amino-1H-pyrazol-1-yl)- |
| | | 1,3,4-thiadiazol-2-yl)-4-(2,6- |
| | | dimethoxyphenyl)-2-oxo-3-(2- |
| | | (piperidin-1-yl)ethoxy)-2H-pyran- |
| | | 6-carboxamide |
| 70 | TA | 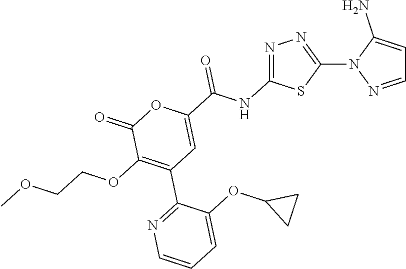 [SMILES: COCCOc1c(-c2ncccc2OC2CC2)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(3- |
| | | cyclopropoxypyridin-2-yl)-3-(2- |
| | | methoxyethoxy)-2-oxo-2H-pyran- |
| | | 6-carboxamide |
| 71A* | 46 | 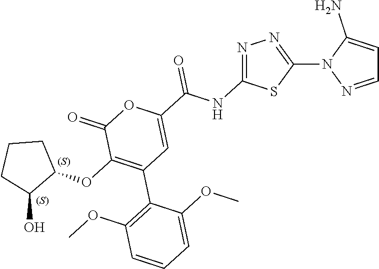 |
| | | N-(5-(5-amino-1H-pyrazol-1-yl)- |
| | | 1,3,4-thiadiazol-2-yl)-4-(2,6- |
| | | dimethoxyphenyl)-3-(((1S,2S)-2- |
| | | hydroxycyclopentyl)oxy)-2-oxo- |
| | | 2H-pyran-6-carboxamide |
| 71B* | 46 | 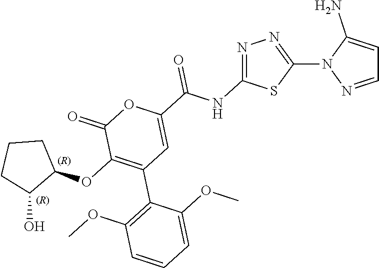 |
| | | N-(5-(5-amino-1H-pyrazol-1-yl)- |
| | | 1,3,4-thiadiazol-2-yl)-4-(2,6- |
| | | dimethoxyphenyl)-3-(((1R,2R)-2- |
| | | hydroxycyclopentyl)oxy)-2-oxo- |
| | | 2H-pyran-6-carboxamide |
| 71- Ac | 46 | 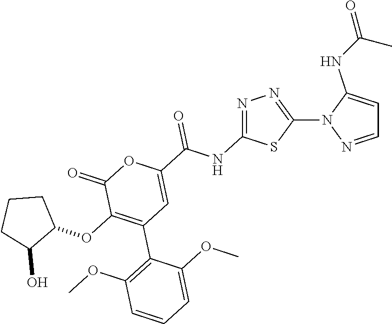 |
| | | N-(5-(5-acetamido-1H-pyrazol-1- |
| | | yl)-1,3,4-thiadiazol-2-yl)-4-(2,6- |
| | | dimethoxyphenyl)-3-(((1,2-trans)- |
| | | 2-hydroxycyclopentyl)oxy)-2-oxo- |
| | | 2H-pyran-6-carboxamide |
| 72 | TA | 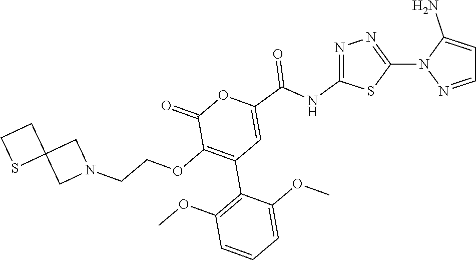 [SMILES: COc1cccc(OC)c1-c1cc(C(=O)Nc2nnc(-n3nccc3N)s2)oc(=O)c1OCCN1CC2(CCS2)C1] |
| | | 3-(2-(1-thia-6-azaspiro[3.3]heptan- |
| | | 6-yl)ethoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 73 | 47 | 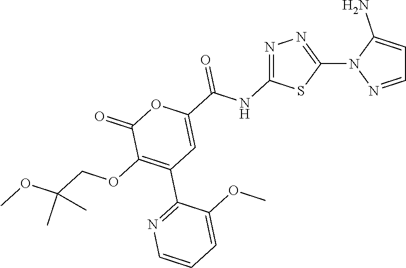 [SMILES: COc1cccnc1-c1cc(C(=O)Nc2nnc(-n3nccc3N)s2)oc(=O)c1OCC(C)(C)OC] |
| | | N-(5-(5-amino-1H-pyrazol-1-yl)- |
| | | 1,3,4-thiadiazol-2-yl)-3-(2- |
| | | methoxy-2-methylpropoxy)-4-(3- |
| | | methoxypyridin-2-yl)-2-oxo-2H- |
| | | pyran-6-carboxamide |
| 73- Ac | 47 | 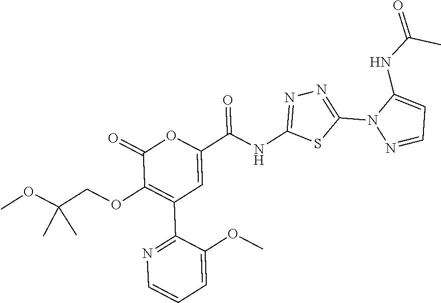 [SMILES: COc1cccnc1-c1cc(C(=O)Nc2nnc(-n3nccc3NC(C)=O)s2)oc(=O)c1OCC(C)(C)OC] |
| | | N-(5-(5-acetamido-1H-pyrazol-1- |
| | | yl)-1,3,4-thiadiazol-2-yl)-3-(2- |
| | | methoxy-2-methylpropoxy)-4-(3- |
| | | methoxypyridin-2-yl)-2-oxo-2H- |
| | | pyran-6-carboxamide |
| 74 | 48 | 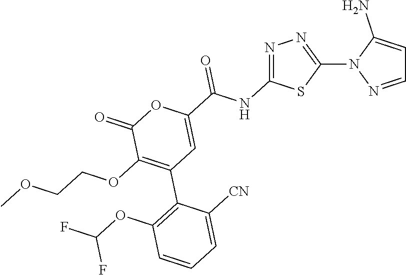 [SMILES: COCCOc1c(-c2c(C#N)cccc2OC(F)F)cc(C(=O)Nc2nnc(-n3nccc3N)s2)oc1=O] |
| | | N-(5-(5-amino-1H-pyrazol-1-yl)- |
| | | 1,3,4-thiadiazol-2-yl)-4-(2-cyano- |
| | | 6-(difluoromethoxy)phenyl)-3-(2- |
| | | methoxyethoxy)-2-oxo-2H-pyran- |
| | | 6-carboxamide |
| 74- Ac | 48 | 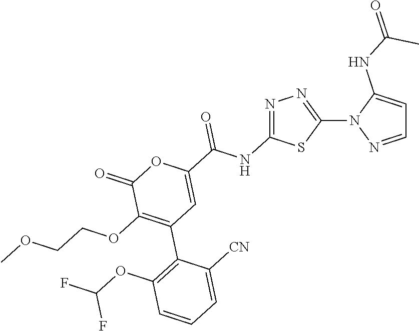 [SMILES: COCCOc1c(-c2c(C#N)cccc2OC(F)F)cc(C(=O)Nc2nnc(-n3nccc3NC(C)=O)s2)oc1=O] |
| | | N-(5-(5-acetamido-1H-pyrazol-1- |
| | | yl)-1,3,4-thiadiazol-2-yl)-4-(2- |
| | | cyano-6-(difluoromethoxy)phenyl)- |
| | | 3-(2-methoxyethoxy)-2-oxo-2H- |
| | | pyran-6-carboxamide |
| 75 | TA | 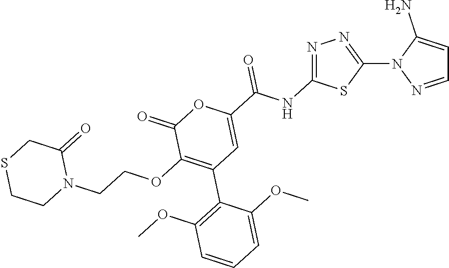 [SMILES: COc1cccc(OC)c1-c1cc(C(=O)Nc2nnc(-n3nccc3N)s2)oc(=O)c1OCCN1CCSCC1=O] |
| | | N-(5-(5-amino-1H-pyrazol-1-yl)- |
| | | 1,3,4-thiadiazol-2-yl)-4-(2,6- |
| | | dimethoxyphenyl)-2-oxo-3-(2-(3- |
| | | oxothiomorpholino)ethoxy)-2H- |
| | | pyran-6-carboxamide |
| 76A* | TA | 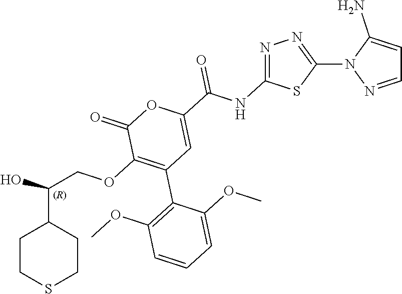 |
| | | (R)-N-(5-(5-amino-1H-pyrazol-1- |
| | | yl)-1,3,4-thiadiazol-2-yl)-4-(2,6- |
| | | dimethoxyphenyl)-3-(2-hydroxy-2- |
| | | (tetrahydro-2H-thiopyran-4- |
| | | yl)ethoxy)-2-oxo-2H-pyran-6- |
| | | carboxamide |
| 76B* | TA | 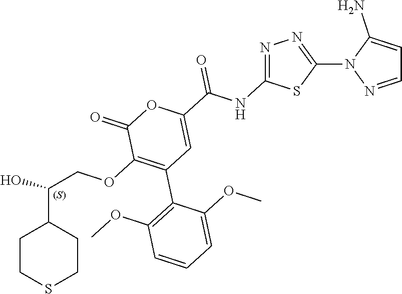 |
| | | (S)-N-(5-(5-amino-1H-pyrazol-1- |
| | | yl)-1,3,4-thiadiazol-2-yl)-4-(2,6- |
| | | dimethoxyphenyl)-3-(2-hydroxy-2- |
| | | (tetrahydro-2H-thiopyran-4- |
| | | yl)ethoxy)-2-oxo-2H-pyran-6- |
| | | carboxamide |
| 77A* | TA | 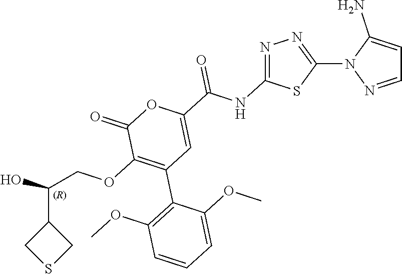 |
| | | (R)-N-(5-(5-amino-1H-pyrazol-1- |
| | | yl)-1,3,4-thiadiazol-2-yl)-4-(2,6- |
| | | dimethoxyphenyl)-3-(2-hydroxy-2- |
| | | (thietan-3-yl)ethoxy)-2-oxo-2H- |
| | | pyran-6-carboxamide |
| 77B* | TA | 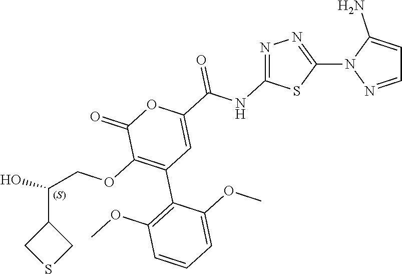 |
| | | (S)-N-(5-(5-amino-1H-pyraozl-1- |
| | | yl)-1,3,4-thiadiazol-2-yl)-4-(2,6- |
| | | dimethoxyphenyl)-3-(2-hydroxy-2- |
| | | (thietan-3-yl)ethoxy)-2-oxo-2H- |
| | | pyran-6-carboxamide |
| 78A* | TA | 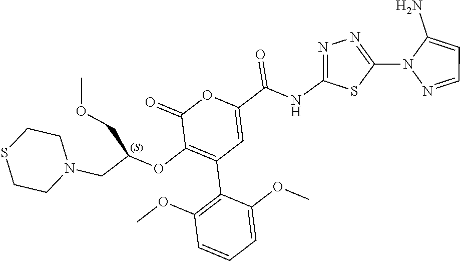 |
| | | (S)-N-(5-(5-amino-1H-pyrazol-1- |
| | | yl)-1,3,4-thiadiazol-2-yl)-4-(2,6- |
| | | dimethoxyphenyl)-3-((1-methoxy- |
| | | 3-thiomorpholinopropan-2-yl)oxy)- |
| | | 2-oxo-2H-pyran-6-carboxamide |
| 78B* | TA | 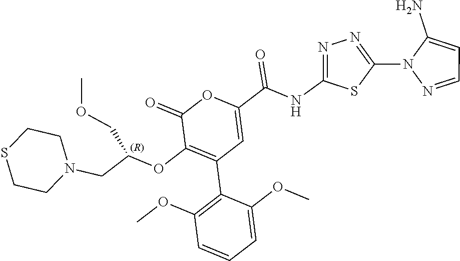 |
| | | (R)-N-(5-(5-amino-1H-pyrazol-1- |
| | | yl)-1,3,4-thiadiazol-2-yl)-4-(2,6- |
| | | dimethoxyphenyl)-3-((1-methoxy- |
| | | 3-thiomorpholinopropan-2-yl)oxy)- |
| | | 2-oxo-2H-pyran-6-carboxamide |
| 79A* | TA | 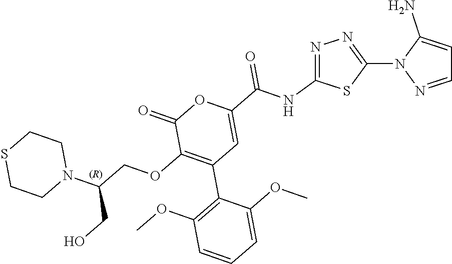 |
| | | (R)-N-(5-(5-amino-1H-pyrazol-1- |
| | | yl)-1,3,4-thiadiazol-2-yl)-4-(2,6- |
| | | dimethoxyphenyl)-3-(3-hydroxy-2- |
| | | thiomorpholinopropoxy)-2-oxo- |
| | | 2H-pyran-6-carboxamide |
| 79B* | TA | 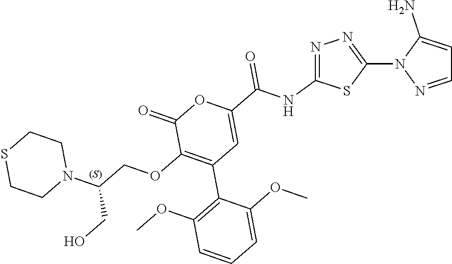 |
| | | (S)-N-(5-(5-amino-1H-pyrazol-1- |
| | | yl)-1,3,4-thiadiazol-2-yl)-4-(2,6- |
| | | dimethoxyphenyl)-3-(3-hydroxy-2- |
| | | thiomorpholinopropoxy)-2-oxo- |
| | | 2H-pyran-6-carboxamide |
| 80A* | TA | 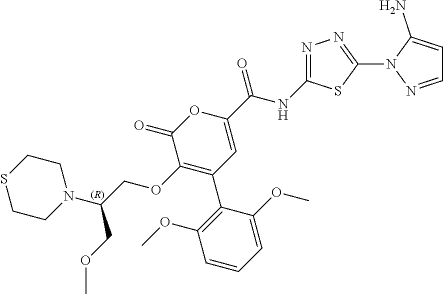 |
| | | (R)-N-(5-(5-amino-1H-pyrazol-1- |
| | | yl)-1,3,4-thiadiazol-2-yl)-4-(2,6- |
| | | dimethoxyphenyl)-3-(3-methoxy-2- |
| | | thiomorpholinopropoxy)-2-oxo- |
| | | 2H-pyran-6-carboxamide |
| 80B* | TA | 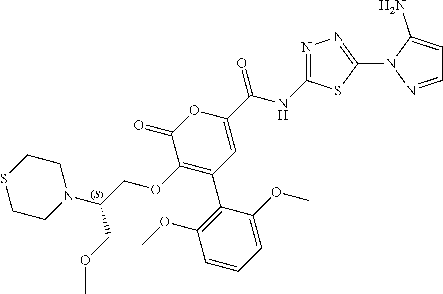 |
| | | (S)-N-(5-(5-amino-1H-pyrazol-1- |
| | | yl)-1,3,4-thiadiazol-2-yl)-4-(2,6- |
| | | dimethoxyphenyl)-3-(3-methoxy-2- |
| | | thiomorpholinopropoxy)-2-oxo- |
| | | 2H-pyran-6-carboxamide |
| 81A* | TA | 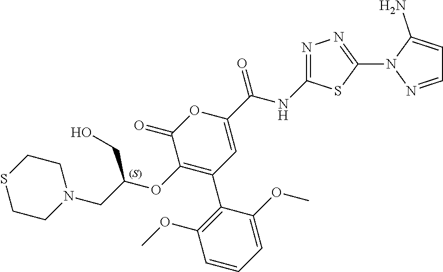 |
| | | (S)-N-(5-(5-amino-1H-pyrazol-1- |
| | | yl)-1,3,4-thiadiazol-2-yl)-4-(2,6- |
| | | dimethoxyphenyl)-3-((1-hydroxy- |
| | | 3-thiomorpholinopropan-2-yl)oxy)- |
| | | 2-oxo-2H-pyran-6-carboxamide |
| 81B* | TA | 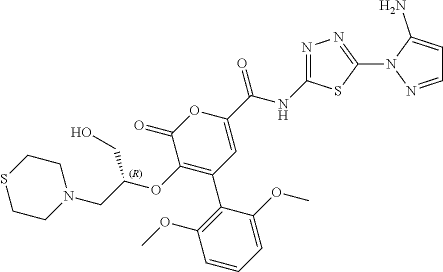 |
| | | (R)-N-(5-(5-amino-1H-pyrazol-1- |
| | | yl)-1,3,4-thiadiazol-2-yl)-4-(2,6- |
| | | dimethoxyphenyl)-3-((1-hydroxy- |
| | | 3-thiomorpholinopropan-2-yl)oxy)- |
| | | 2-oxo-2H-pyran-6-carboxamide |
| 82A* | TA | 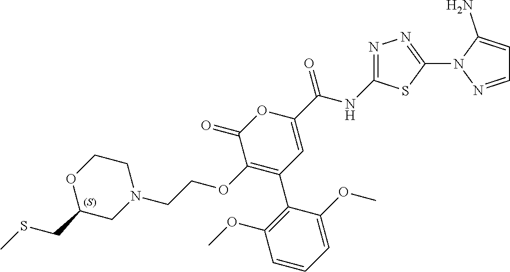 |
| | | (S)-N-(5-(5-amino-1H-pyrazol-1- |
| | | yl)-1,3,4-thiadiazol-2-yl)-4-(2,6- |
| | | dimethoxyphenyl)-3-(2-(2- |
| | | ((methylthio)methyl)morpholino)eth- |
| | | oxy)-2-oxo-2H-pyran-6- |
| | | carboxamide |
| 82B* | TA | 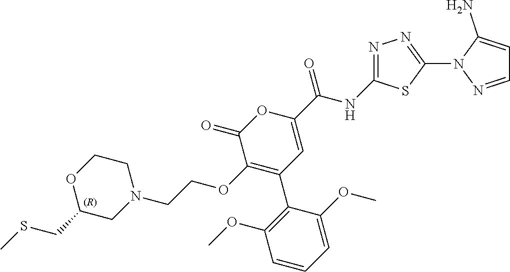 |
| | | (R)-N-(5-(5-amino-1H-pyrazol-1- |
| | | yl)-1,3,4-thiadiazol-2-yl)-4-(2,6- |
| | | dimethoxyphenyl)-3-(2-(2- |
| | | ((methylthio)methyl)morpholino)eth- |
| | | oxy)-2-oxo-2H-pyran-6- |
| | | carboxamide |
| 83A* | TA | 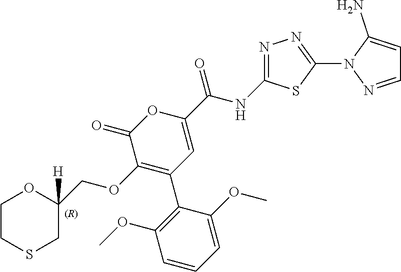 |
| | | (R)-3-((1,4-oxathian-2- |
| | | yl)methoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 83B* | TA | 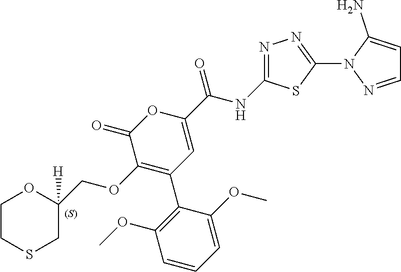 |
| | | (S)-3-((1,4-oxathian-2- |
| | | yl)methoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 84A* | TA | 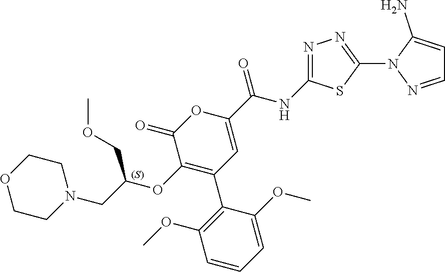 |
| | | (S)-N-(5-(5-amino-1H-pyrazol-1- |
| | | yl)-1,3,4-thiadiazol-2-yl)-4-(2,6- |
| | | dimethoxyphenyl)-3-((1-methoxy- |
| | | 3-morpholinopropan-2-yl)oxy)-2- |
| | | oxo-2H-pyran-6-carobxamide |
| 84B* | TA | 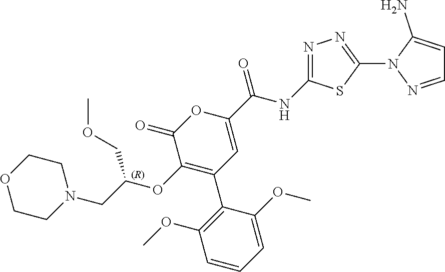 |
| | | (R)-N-(5-(5-amino-1H-pyrazol-1- |
| | | yl)-1,3,4-thiadiazol-2-yl)-4-(2,6- |
| | | dimethoxyphenyl)-3-((1-methoxy- |
| | | 3-morpholinopropan-2-yl)oxy)-2- |
| | | oxo-2H-pyran-6-carboxamide |
| 85A* | TA | 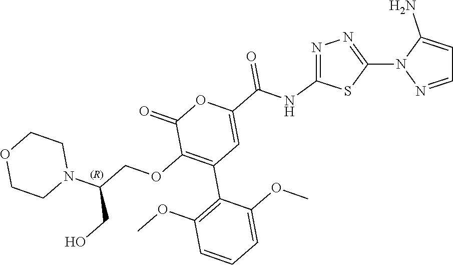 |
| | | (R)-N-(5-(5-amino-1H-pyrazol-1- |
| | | yl)-1,3,4-thiadiazol-2-yl)-4-(2,6- |
| | | dimethoxyphenyl)-3-(3-hydroxy-2- |
| | | morpholinopropoxy)-2-oxo-2H- |
| | | pyran-6-carboxamide |
| 85B* | TA | 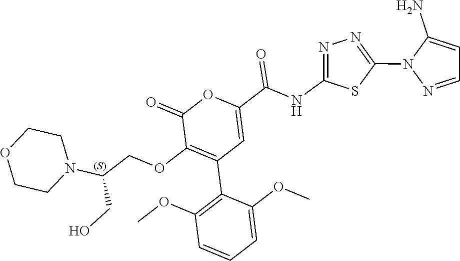 |
| | | (S)-N-(5-(5-amino-1H-pyrazol-1- |
| | | yl)-1,3,4-thiadiazol-2-yl)-4-(2,6- |
| | | dimethoxyphenyl)-3-(3-hydroxy-2- |
| | | morpholinopropoxy)-2-oxo-2H- |
| | | pyran-6-carboxamide |
| 86A* | TA | 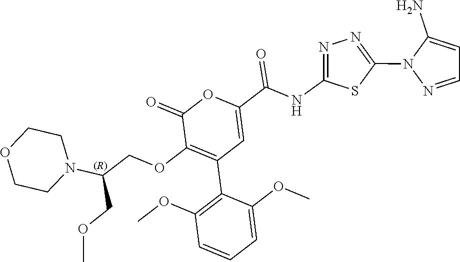 |
| | | (R)-N-(5-(5-amino-1H-pyrazol-1- |
| | | yl)-1,3,4-thiadiazol-2-yl)-4-(2,6- |
| | | dimethoxyphenyl)-3-(3-methoxy-2- |
| | | morpholinopropoxy)-2-oxo-2H- |
| | | pyran-6-carboxamide |
| 86B* | TA | 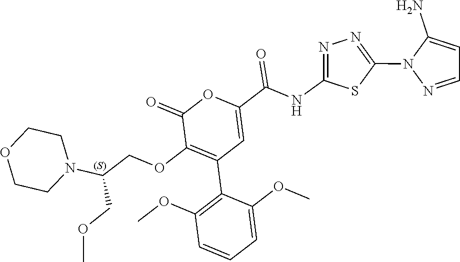 |
| | | (S)-N-(5-(5-amino-1H-pyrazol-1- |
| | | yl)-1,3,4-thiadiazol-2-yl)-4-(2,6- |
| | | dimethoxyphenyl)-3-(3-methoxy-2- |
| | | morpholinopropoxy)-2-oxo-2H- |
| | | pyran-6-carboxamide |
| 87A* | TA | 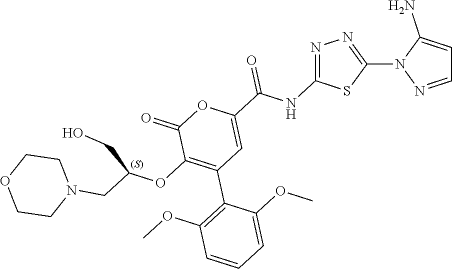 |
| | | (S)-N-(5-(5-amino-1H-pyrazol-1- |
| | | yl)-1,3,4-thiadiazol-2-yl)-4-(2,6- |
| | | dimethoxyphenyl)-3-((1-hydroxy- |
| | | 3-morpholinopropan-2-yl)oxy)-2- |
| | | oxo-2H-pyran-6-carboxamide |
| 87B* | TA | 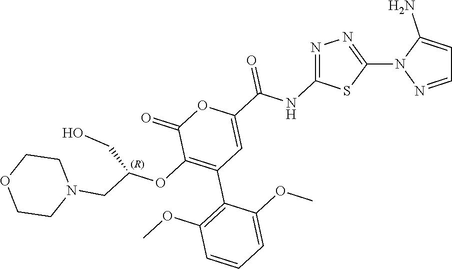 |
| | | (R)-N-(5-(5-amino-1H-pyrazol-1- |
| | | yl)-1,3,4-thiadiazol-2-yl)-4-(2,6- |
| | | dimethoxyphenyl)-3-((1-hydroxy- |
| | | 3-morpholinopropan-2-yl)oxy)-2- |
| | | oxo-2H-pyran-6-carboxamide |
| 88 | TA | 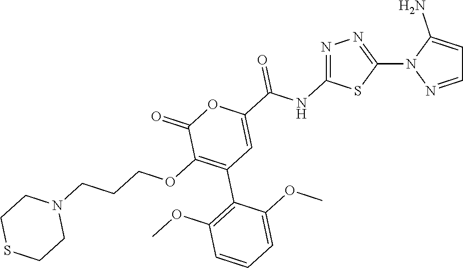 [SMILES: COc1cccc(OC)c1-c1cc(C(=O)Nc2nnc(-n3nccc3N)s2)oc(=O)c1OCCCN1CCSCC1] |
| | | N-(5-(5-amino-1H-pyrazol-1-yl)- |
| | | 1,3,4-thiadiazol-2-yl)-4-(2,6- |
| | | dimethoxyphenyl)-2-oxo-3-(3- |
| | | thiomorpholinopropoxy)-2H- |
| | | pyran-6-carboxamide |
| 89 | TA | 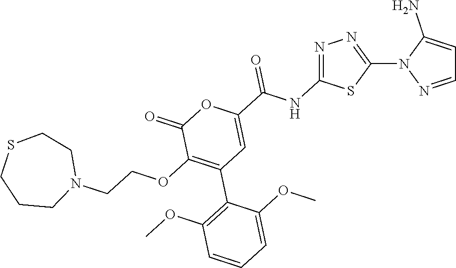 [SMILES: COc1cccc(OC)c1-c1cc(C(=O)Nc2nnc(-n3nccc3N)s2)oc(=O)c1OCCN1CCCSCC1] |
| | | 3-(2-(1,4-thiazepan-4-yl)ethoxy)- |
| | | N-(5-(5-amino-1H-pyrazol-1-yl)- |
| | | 1,3,4-thiadiazol-2-yl)-4-(2,6- |
| | | dimethoxyphenyl)-2-oxo-2H- |
| | | pyran-6-carboxamide |
| 90 | TA | 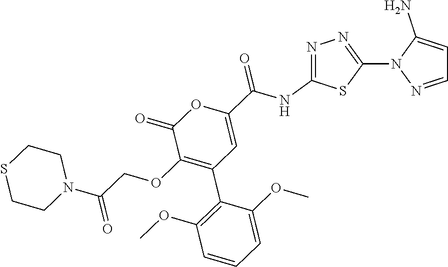 [SMILES: COc1cccc(OC)c1-c1cc(C(=O)Nc2nnc(-n3nccc3N)s2)oc(=O)c1OCC(=O)N1CCSCC1] |
| | | N-(5-(5-amino-1H-pyrazol-1-yl)- |
| | | 1,3,4-thiadiazol-2-yl)-4-(2,6- |
| | | dimethoxyphenyl)-2-oxo-3-(2-oxo- |
| | | 2-thiomorpholinoethoxy)-2H- |
| | | pyran-6-carboxamide |
| 91A* | TA | 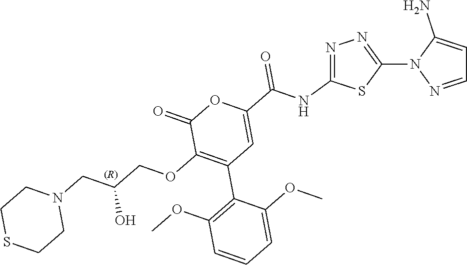 |
| | | (R)-N-(5-(5-amino-1H-pyrazol-1- |
| | | yl)-1,3,4-thiadiazol-2-yl)-4-(2,6- |
| | | dimethoxyphenyl)-3-(2-hydroxy-3- |
| | | thiomorpholinopropoxy)-2-oxo- |
| | | 2H-pyran-6-carboxamide |
| 91B* | TA | 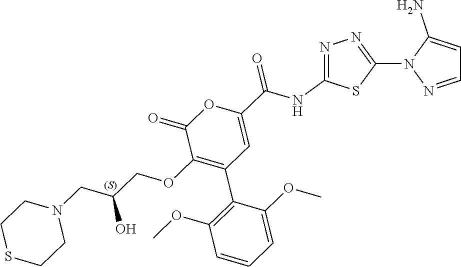 |
| | | (S)-N-(5-(5-amino-1H-pyrazol-1- |
| | | yl)-1,3,4-thiadiazol-2-yl)-4-(2,6- |
| | | dimethoxyphenyl)-3-(2-hydroxy-3- |
| | | thiomorpholinopropoxy)-2-oxo- |
| | | 2H-pyran-6-carboxamide |
| 92A* | TA | 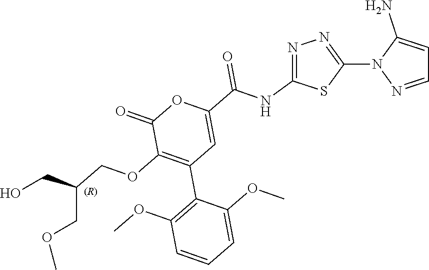 |
| | | (R)-N-(5-(5-amino-1H-pyrazol-1- |
| | | yl)-1,3,4-thiadiazol-2-yl)-4-(2,6- |
| | | dimethoxyphenyl)-3-(3-hydroxy-2- |
| | | (methoxymethyl)propoxy)-2-oxo- |
| | | 2H-pyran-6-carboxamide |
| 92B* | TA | 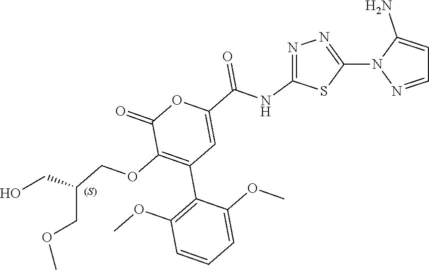 |
| | | (S)-N-(5-(5-amino-1H-pyrazol-1- |
| | | yl)-1,3,4-thiadiazol-2-yl)-4-(2,6- |
| | | dimethoxyphenyl)-3-(3-hydroxy-2- |
| | | (methoxymethyl)propoxy)-2-oxo- |
| | | 2H-pyran-6-carboxamide |
| 93A* | TA | 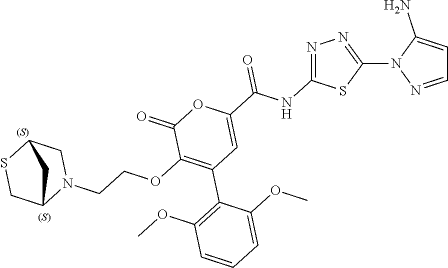 |
| | | 3-(2-((1S,4S)-2-thia-5- |
| | | azabicyclo[2.2.1]heptan-5- |
| | | yl)ethoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 93B* | TA | 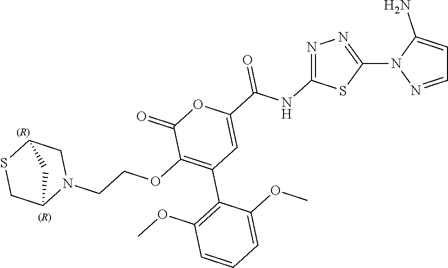 |
| | | 3-(2-((1R,4R)-2-thia-5- |
| | | azabicyclo[2.2.1]heptan-5- |
| | | yl)ethoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 94 | TA | 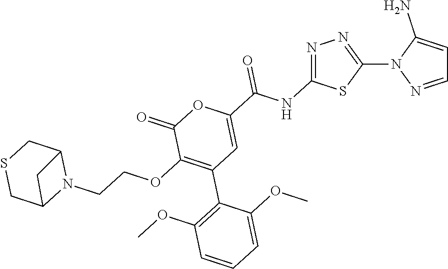 [SMILES: COc1cccc(OC)c1-c1cc(C(=O)Nc2nnc(-n3nccc3N)s2)oc(=O)c1OCCN1C2CSCC1C2] |
| | | 3-(2-(3-thia-6- |
| | | azabicyclo[3.1.1]heptan-6- |
| | | yl)ethoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide |
| 95 | TA | 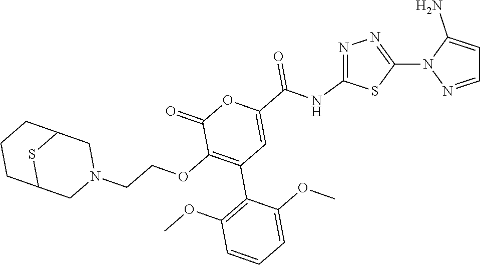 [SMILES: COc1cccc(OC)c1-c1cc(C(=O)Nc2nnc(-n3nccc3N)s2)oc(=O)c1OCCN1CC2CCCC(C1)S2] |
| | | 3-(2-(9-thia-3- |
| | | azabicyclo[3.3.1]nonan-3- |
| | | yl)ethoxy)-N-(5-(5-amino-1H- |
| | | pyrazol-1-yl)-1,3,4-thiadiazol-2- |
| | | yl)-4-(2,6-dimethoxyphenyl)-2- |
| | | oxo-2H-pyran-6-carboxamide/ |
| 96A* | TA | 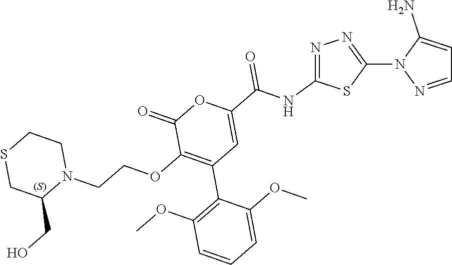 |
| | | (S)-N-(5-(5-amino-1H-pyrazol-1- |
| | | yl)-1,3,4-thiadiazol-2-yl)-4-(2,6- |
| | | dimethoxyphenyl)-3-(2-(3- |
| | | (hydroxymethyl)thiomorpholino)eth- |
| | | oxy)-2-oxo-2H-pyran-6- |
| | | carboxamide |
| 96B* | TA | 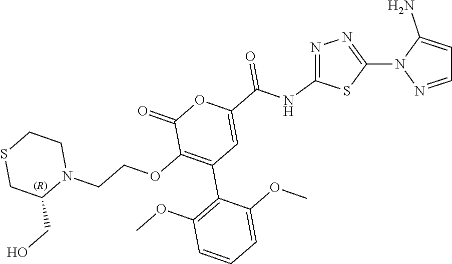 |
| | | (R)-N-(5-(5-amino-1H-pyrazol-1- |
| | | yl)-1,3,4-thiadiazol-2-yl)-4-(2,6- |
| | | dimethoxyphenyl)-3-(2-(3- |
| | | (hydroxymethyl)thiomorpholino)eth- |
| | | oxy)-2-oxo-2H-pyran-6- |
| | | carboxamide |
| 97A* | TA | 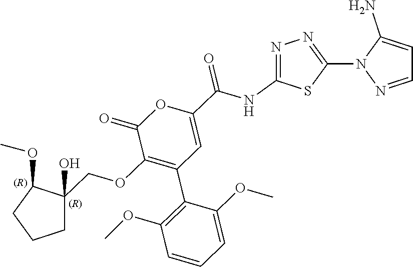 |
| | | N-(5-(5-amino-1H-pyrazol-1-yl)- |
| | | 1,3,4-thiadiazol-2-yl)-4-(2,6- |
| | | dimethoxyphenyl)-3-(((1R,2R)-1- |
| | | hydroxy-2- |
| | | methoxycyclopentyl)methoxy)-2- |
| | | oxo-2H-pyran-6-carboxamide |
| 97B* | TA | 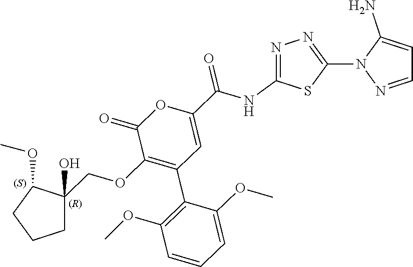 |
| | | N-(5-(5-amino-1H-pyrazol-1-yl)- |
| | | 1,3,4-thiadiazol-2-yl)-4-(2,6- |
| | | dimethoxyphenyl)-3-(((1R,2S)-1- |
| | | hydroxy-2- |
| | | methoxycyclopentyl)methoxy)-2- |
| | | oxo-2H-pyran-6-carboxamide |
| 97C* | TA | 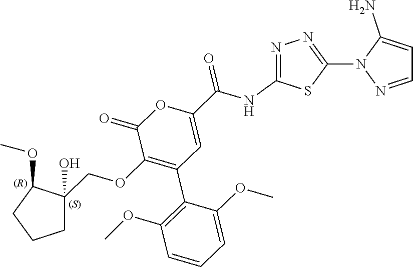 |
| | | N-(5-(5-amino-1H-pyrazol-1-yl)- |
| | | 1,3,4-thiadiazol-2-yl)-4-(2,6- |
| | | dimethoxyphenyl)-3-(((1S,2R)-1- |
| | | hydroxy-2- |
| | | methoxycyclopentyl)methoxy)-2- |
| | | oxo-2H-pyran-6-carboxamide |
| 97D* | TA | 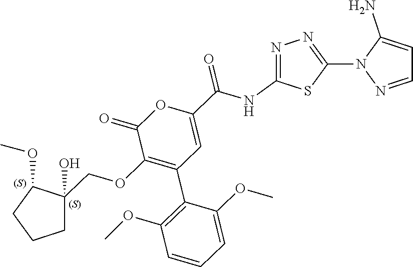 |
| | | N-(5-(5-amino-1H-pyrazol-1-yl)- |
| | | 1,3,4-thiadiazol-2-yl)-4-(2,6- |
| | | dimethoxyphenyl)-3-(((1S,2S)-1- |
| | | hydroxy-2- |
| | | methoxycyclopentyl)methoxy)-2- |
| | | oxo-2H-pyran-6-carboxamide |
| 98A* | TA | 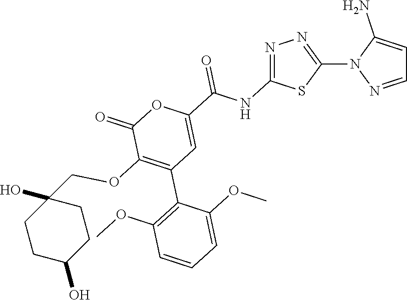 [SMILES: COc1cccc(OC)c1-c1cc(C(=O)Nc2nnc(-n3nccc3N)s2)oc(=O)c1OC[C@]1(O)CC[C@@H](O)CC1] |
| | | N-(5-(5-amino-1H-pyrazol-1-yl)- |
| | | 1,3,4-thiadiazol-2-yl)-3-(((1s,4s)- |
| | | 1,4- |
| | | dihydroxycyclohexyl)methoxy)-4- |
| | | (2,6-dimethoxyphenyl)-2-oxo-2H- |
| | | pyran-6-carboxamide |
| 98B* | TA | 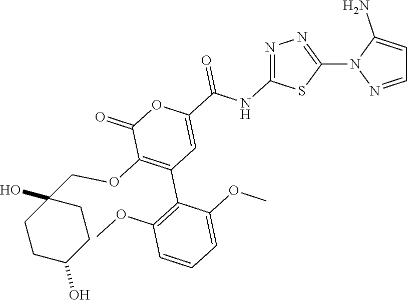 [SMILES: COc1cccc(OC)c1-c1cc(C(=O)Nc2nnc(-n3nccc3N)s2)oc(=O)c1OC[C@]1(O)CC[C@H](O)CC1] |
| | | N-(5-(5-amino-1H-pyrazol-1-yl)- |
| | | 1,3,4-thiadiazol-2-yl)-3-(((1r,4r)- |
| | | 1,4- |
| | | dihydroxycyclohexyl)methoxy)-4- |
| | | (2,6-dimethoxyphenyl)-2-oxo-2H- |
| | | pyran-6-carboxamide |
| 99A* | TA | 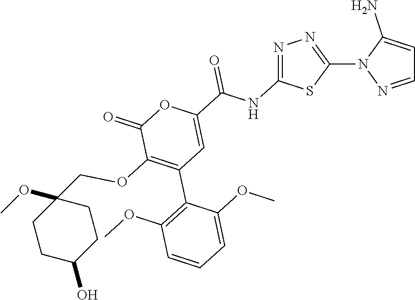 [SMILES: COc1cccc(OC)c1-c1cc(C(=O)Nc2nnc(-n3nccc3N)s2)oc(=O)c1OC[C@]1(OC)CC[C@@H](O)CC1] |
| | | N-(5-(5-amino-1H-pyrazol-1-yl)- |
| | | 1,3,4-thiadiazol-2-yl)-4-(2,6- |
| | | dimethoxyphenyl)-3-(((1s,4s)-4- |
| | | hydroxy-1- |
| | | methoxycyclohexyl)methoxy)-2- |
| | | oxo-2H-pyran-6-carboxamide |
| 99B* | TA | 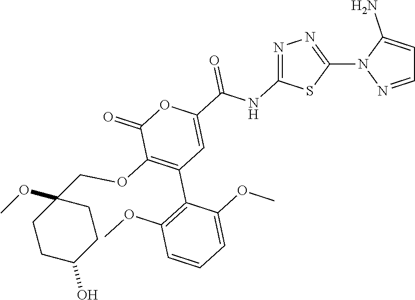 [SMILES: COc1cccc(OC)c1-c1cc(C(=O)Nc2nnc(-n3nccc3N)s2)oc(=O)c1OC[C@]1(OC)CC[C@H](O)CC1] |
| | | N-(5-(5-amino-1H-pyrazol-1-yl)- |
| | | 1,3,4-thiadiazol-2-yl)-4-(2,6- |
| | | dimethoxyphenyl)-3-(((1r,4r)-4- |
| | | hydroxy-1- |
| | | methoxycyclohexyl)methoxy)-2- |
| | | oxo-2H-pyran-6-carobxamide |
| 100A* | TA | 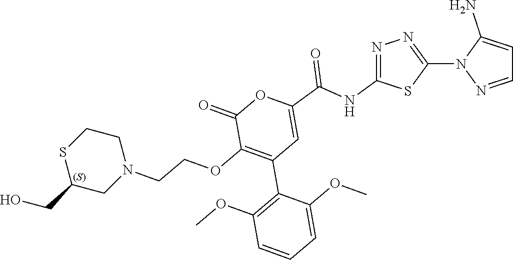 |
| | | (S)-N-(5-(5-amino-1H-pyrazol-1- |
| | | yl)-1,3,4-thiadiazol-2-yl)-4-(2,6- |
| | | dimethoxyphenyl)-3-(2-(2- |
| | | (hydroxymethyl)thiomorpholino)eth- |
| | | oxy)-2-oxo-2H-pyran-6- |
| | | carboxamide |
| 100B* | TA | 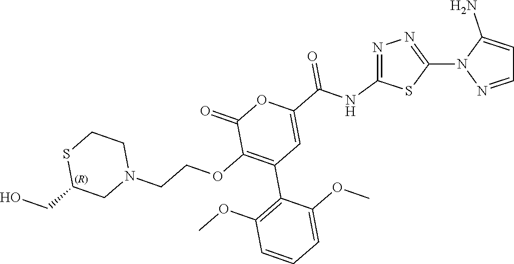 |
| | | (R)-N-(5-(5-amino-1H-pyrazol-1- |
| | | yl)-1,3,4-thiadiazol-2-yl)-4-(2,6- |
| | | dimethoxyphenyl)-3-(2-(2- |
| | | (hydroxymethyl)thiomorpholino)eth- |
| | | oxy)-2-oxo-2H-pyran-6- |
| | | carboxamide |
| 101A* | TA | 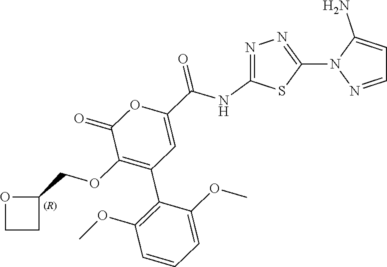 |
| | | (R)-N-(5-(5-amino-1H-pyrazol-1- |
| | | yl)-1,3,4-thiadiazol-2-yl)-4-(2,6- |
| | | dimethoxyphenyl)-3-(oxetan-2- |
| | | ylmethoxy)-2-oxo-2H-pyran-6- |
| | | carboxamide |
| 101B* | TA | 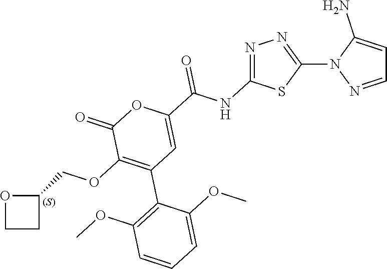 |
| | | (S)-N-(5-(5-amino-1H-pyrazol-1- |
| | | yl)-1,3,4-thiadiazol-2-yl)-4-(2,6- |
| | | dimethoxyphenyl)-3-(oxetan-2- |
| | | ylmethoxy)-2-oxo-2H-pyran-6- |
| | | carboxamide |
| 102A* | TA | 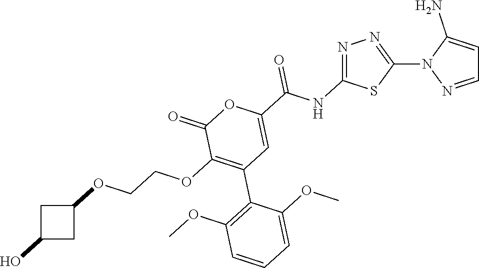 |
| | | N-(5-(5-amino-1H-pyrazol-1-yl)- |
| | | 1,3,4-thiadiazol-2-yl)-4-(2,6- |
| | | dimethoxyphenyl)-3-(2-((1s,3s)-3- |
| | | hydroxycyclobutoxy)ethoxy)-2- |
| | | oxo-2H-pyran-6-carobxamide |
| 102B* | TA | 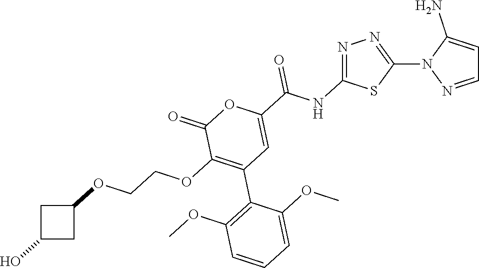 |
| | | N-(5-(5-amino-1H-pyrazol-1-yl)- |
| | | 1,3,4-thiadiazol-2-yl)-4-(2,6- |
| | | dimethoxyphenyl)-3-(2-((1r,3r)-3- |
| | | hydroxycyclobutoxy)ethoxy)-2- |
| | | oxo-2H-pyran-6-carboxamide |
| 103 | TA | 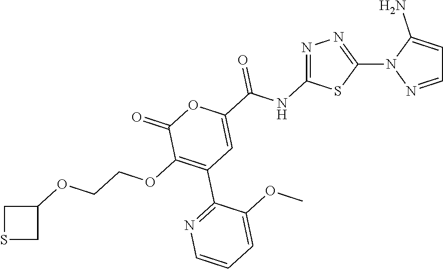 [SMILES: COc1cccnc1-c1cc(C(=O)Nc2nnc(-n3nccc3N)s2)oc(=O)c1OCCOC1CSC1] |
| | | N-(5-(5-amino-1H-pyrazol-1-yl)- |
| | | 1,3,4-thiadiazol-2-yl)-4-(3- |
| | | methoxypyridin-2-yl)-2-oxo-3-(2- |
| | | (thietan-3-yloxy)ethoxy)-2H- |
| | | pyran-6-carboxamide |
| 104 | TA | 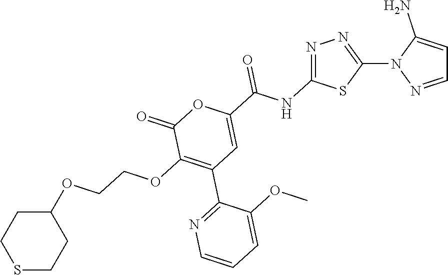 [SMILES: COc1cccnc1-c1cc(C(=O)Nc2nnc(-n3nccc3N)s2)oc(=O)c1OCCOC1CCSCC1] |
| | | N-(5-(5-amino-1H-pyrazol-1-yl)- |
| | | 1,3,4-thiadiazol-2-yl)-4-(3- |
| | | methoxypyridin-2-yl)-2-oxo-3-(2- |
| | | ((tetrahydro-2H-thiopyran-4- |
| | | yl)oxy)ethoxy)-2H-pyran-6- |
| | | carboxamide |
| 105A* | TA | 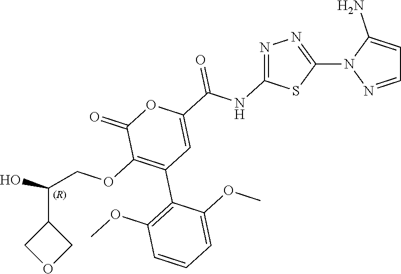 |
| | | (R)-N-(5-(5-amino-1H-pyrazol-1- |
| | | yl)-1,3,4-thiadiazol-2-yl)-4-92,6- |
| | | dimethoxyphenyl)-3-(2-hydroxy-2- |
| | | (oxetan-3-yl)ethoxy)-2-oxo-2H- |
| | | pyran-6-carboxamide |
| 105B* | TA | 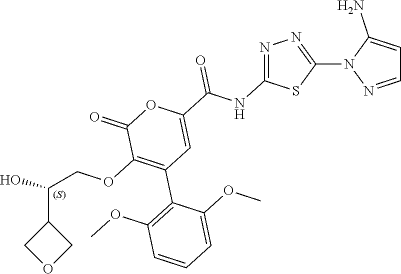 |
| | | (S)-N-(5-(5-amino-1H-pyrazol-1- |
| | | yl)-1,3,4-thiadiazol-2-yl)-4-(2,6- |
| | | dimethoxyphenyl)-3-(2-hydroxy-2- |
| | | (oxetan-3-yl)ethoxy)-2-oxo-2H- |
| | | pyran-6-carboxamide |
| 106A* | TA | 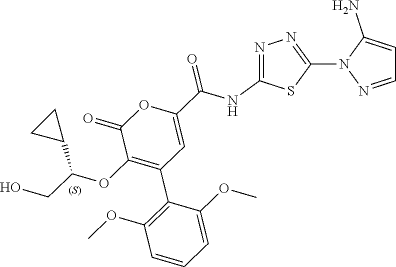 |
| | | (S)-N-(5-(5-amino-1H-pyrazol-1- |
| | | yl)-1,3,4-thiadiazol-2-yl)-3-(1- |
| | | cyclopropyl-2-hydroxyethoxy)-4- |
| | | (2,6-dimethoxyphenyl)-2-oxo-2H- |
| | | pyran-6-carboxamide |
| 106 B* | TA | 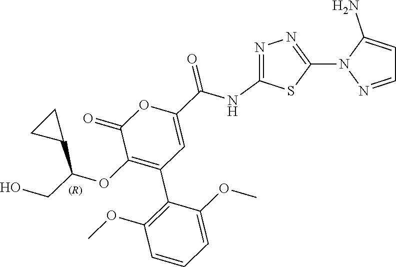 |
| | | (R)-N-(5-(5-amino-1H-pyrazol-1- |
| | | yl)-1,3,4-thiadiazol-2-yl)-3-(1- |
| | | cyclopropyl-2-hydroxyethoxy)-4- |
| | | 2,6-dimethoxyphenyl)-2-oxo-2H- |
| | | pyran-6-carboxamide |
| 55- Ac- OtBu | 40 | 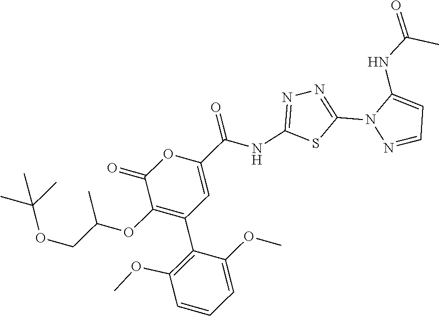 [SMILES: COc1cccc(OC)c1-c1cc(C(=O)Nc2nnc(-n3nccc3NC(C)=O)s2)oc(=O)c1OC(C)COC(C)(C)C] |
| | | N-(5-(5-acetamido-1H-pyrazol-1- |
| | | yl)-1,3,4-thiadiazol-2-yl)-3-((1- |
| | | (tert-butoxy)propan-2-yl)oxy)-4- |
| | | (2,6-dimethoxyphenyl)-2-oxo-2H- |
| | | pyran-6-carboxamide |
| — | — | — |

TABLE 2
Compounds of Formula (I)

| # | Ex | Structure and Name |
|---|---|---|
| 107 A | 49 | 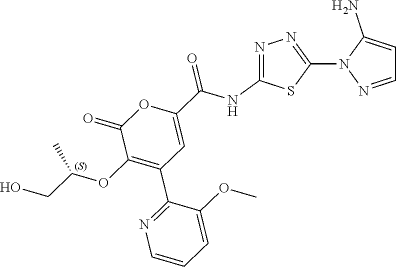 |
| | | (S)-N-(5-(5-amino-1H-pyrazol-1- |
| | | yl)-1,3,4-thiadiazol-2-yl)-3-((1- |
| | | hydroxypropan-2-yl)oxy)-4-(3- |
| | | methoxypyridin-2-yl)-2-oxo-2H- |
| | | pyran-6-carboxamide |
| 107 A- Ac | | 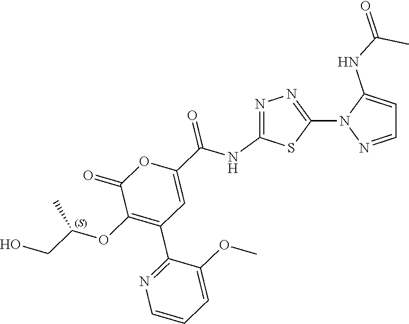 |
| | | (S)-N-(5-(5-acetamido-1H- |
| | | pyrazol-1-yl)-1,3,4-thiadiazol-2- |
| | | yl)-3-((1-hydroxypropan-2- |
| | | yl)oxy)-4-(3-methoxypyridin-2- |
| | | yl)-2-oxo-2H-pyran-- |
| | | carboxamide |
| 107 B | TA | 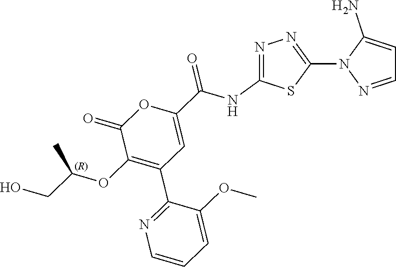 |
| | | (R)-N-(5-(5-amino-1H-pyrazol-1- |
| | | yl)-1,3,4-thiadiazol-2-yl)-3-((1- |
| | | hydroxypropan-2-yl)oxy)-4-(3- |
| | | methoxypyridin-2-yl)-2-oxo-2H- |
| | | pyran-6-carboxamide |
| 107 B- Ac | | 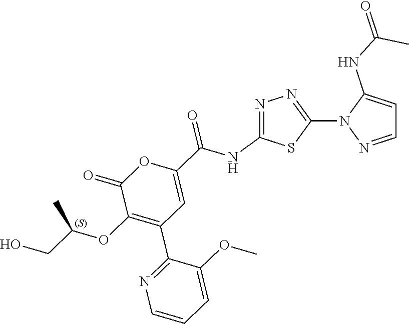 |
| | | (R)-N-(5-(5-acetamido)-1H- |
| | | pyrazol-1-yl)-1,3,4-thiadiazol-2- |
| | | yl)-3-((1-hydroxypropan-2- |
| | | yl)oxy)-4-(3-methoxypyridin-2- |
| | | yl)-2-oxo-2H-pyran-6- |
| | | carboxamide |
| 108 A | 50 | 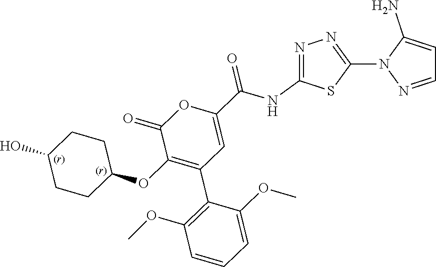 |
| | | N-(5-(5-amino-1H-pyrazol-1-yl)- |
| | | 1,3,4-thiadiazol-2-yl)-4-(2,6- |
| | | dimethoxyphenyl)-3-(((1r,4r)-4- |
| | | hydroxycyclohexyl)oxy)-2-oxo- |
| | | 2H-pyran-6-carboxamide |
| 108 B | TA | 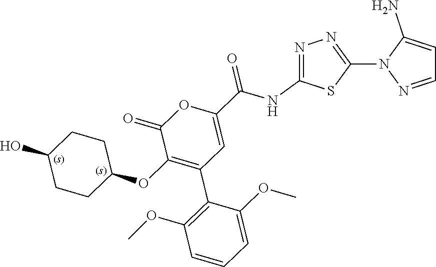 |
| | | N-(5-(5-amino-1H-pyrazol-1-yl)- |
| | | 1,3,4-thiadiazol-2-yl)-4-(2,6- |
| | | dimethoxyphenyl)-3-(((1s,4s)-4- |
| | | hydroxycyclohexyl)oxy)-2-oxo- |
| | | 2H-pyran-6-carboxamide |
| 109 A* | TA | 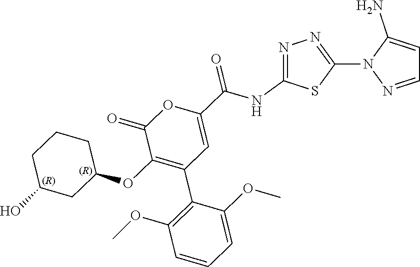 |
| | | N-(5-(5-amino-1H-pyrazol-1-yl)- |
| | | 1,3,4-thiadiazol-2-yl)-4-(2,6- |
| | | dimethoxyphenyl)-3-(((1R,3R)-3- |
| | | hydroxycyclohexyl)oxy)-2-oxo- |
| | | 2H-pyran-6-carboxamide |
| 109 B* | TA | 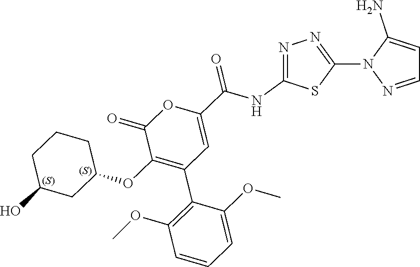 |
| | | N-(5-(5-amino-1H-pyrazol-1-yl)- |
| | | 1,3,4-thiadiazol-2-yl)-4-(2,6- |
| | | dimethoxyphenyl)-3-(((1S,3S)-3- |
| | | hydroxycyclohexyl)oxy)-2-oxo- |
| | | 2H-pyran-6-carboxamide |
| 110 A* | TA | 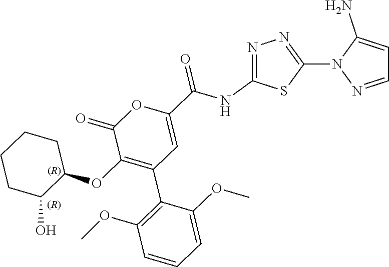 |
| | | N-(5-(5-amino-1H-pyrazol-1-yl)- |
| | | 1,3,4-thiadiazol-2-yl)-4-(2,6- |
| | | dimethoxyphenyl)-3-(((1R,2R)-2- |
| | | hydroxycyclohexyl)oxy)-2-oxo- |
| | | 2H-pyran-6-carboxamide |
| 110 B* | TA | 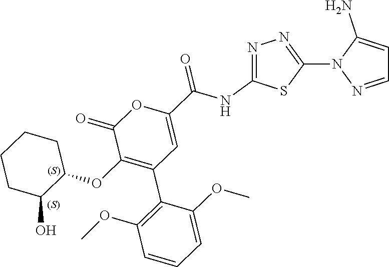 |
| | | N-(5-(5-amino-1H-pyrazol-1-yl)- |
| | | 1,3,4-thiadiazol-2-yl)-4-(2,6- |
| | | dimethoxyphenyl)-3-(((1S,2S)-2- |
| | | hydroxycyclohexyl)oxy)-2-oxo- |
| | | 2H-pyran-6-carboxamide |
| 111 A* | 51 | 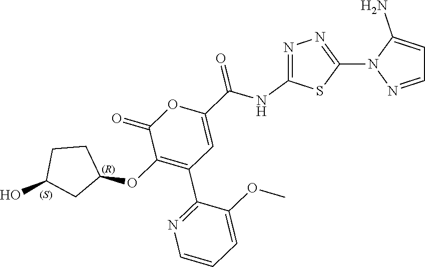 |
| | | N-(5-(5-amino-1H-pyrazol-1-yl)- |
| | | 1,3,4-thiadiazol-2-yl)-3-(((1R,3S)- |
| | | 3-hydroxycyclopentyl)oxy)-4-(3- |
| | | methoxypyridin-2-yl)-2-oxo-2H- |
| | | pyran-6-carboxamide |
| 111 B* | 51 | 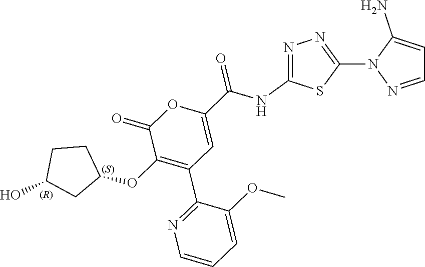 |
| | | N-(5-(5-amino-1H-pyrazol-1-yl)- |
| | | 1,3,4-thiadiazol-2-yl)-3-(((1S,3R)- |
| | | 3-hydroxycyclopentyl)oxy)-4-(3- |
| | | methoxypyridin-2-yl)-2-oxo-2H- |
| | | pyran-6-carboxamide |
| 112 A* | TA | 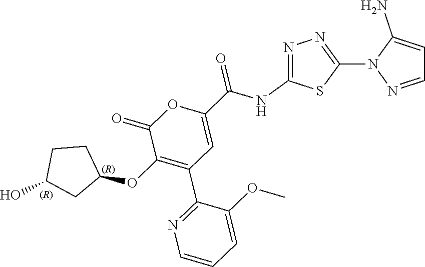 |
| | | N-(5-(5-amino-1H-pyrazol-1-yl)- |
| | | 1,3,4-thiadiazol-2-yl)-3-(((1R,3R)- |
| | | 3-hydroxycyclopentyl)oxy)-4-(3- |
| | | methoxypyridin-2-yl)-2-oxo-2H- |
| | | pyran-6-carboxamide |
| 112 B* | TA | 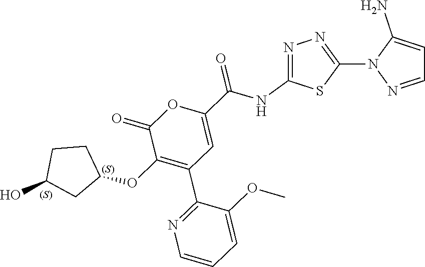 |
| | | N-(5-(5-amino-1H-pyrazol-1-yl)- |
| | | 1,3,4-thiadiazol-2-yl)-3-(((1S,3S)- |
| | | 3-hydroxycyclopentyl)oxy)-4-(3- |
| | | methoxypyridin-2-yl)-2-oxo-2H- |
| | | pyran-6-carboxamide |
| 113- Ac | 52 | 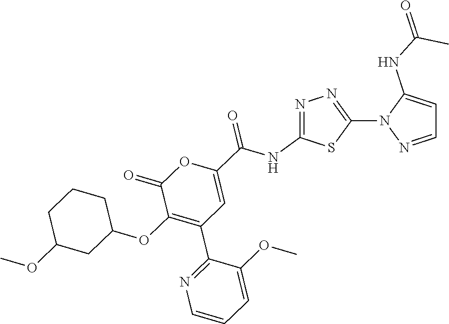 [SMILES: COc1cccnc1-c1cc(C(=O)Nc2nnc(-n3nccc3NC(C)=O)s2)oc(=O)c1OC1CCCC(OC)C1] |
| | | N-(5-(5-acetamido-1H-pyrazol-1- |
| | | yl)-1,3,4-thiadiazol-2-yl)-3-((3- |
| | | methoxycyclohexyl)oxy)-4-(3- |
| | | methoxypyridin-2-yl)-2-oxo-2H- |
| | | pyran-6-carboxamide |
| 113 A* | 52 | 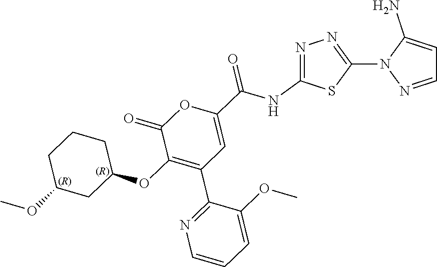 |
| | | N-(5-(5-amino-1H-pyrazol-1-yl)- |
| | | 1,3,4-thiadiazol-2-yl)-3-(((1R,3R)- |
| | | 3-methoxycyclohexyl)oxy)-4-(3- |
| | | methoxypyridin-2-yl)-2-oxo-2H- |
| | | pyran-6-carboxamide |
| 113 B* | 52 | 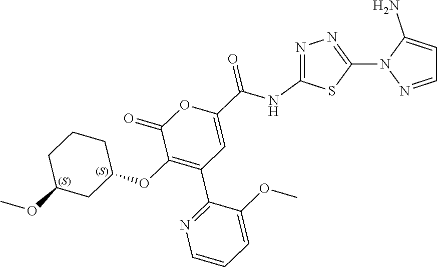 |
| | | N-(5-(5-amino-1H-pyrazol-1-yl)- |
| | | 1,3,4-thiadiazol-2-yl)-3-(((1S,3S)- |
| | | 3-methoxycyclohexyl)oxy)-4-(3- |
| | | methoxypyridin-2-yl)-2-oxo-2H- |
| | | pyran-6-carboxamide |
| 113 C* | 52 | 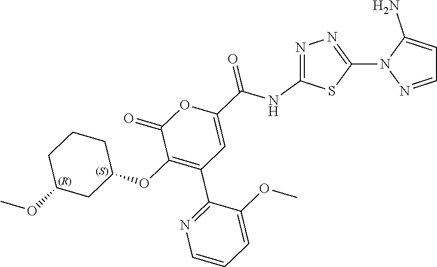 |
| | | N-(5-(5-amino-1H-pyrazol-1-yl)- |
| | | 1,3,4-thiadiazol-2-yl)-3-(((1S,3R)- |
| | | 3-methoxycyclohexyl)oxy)-4-(3- |
| | | methoxypyridin-2-yl)-2-oxo-2H- |
| | | pyran-6-carboxamide |
| 113 D* | 52 | 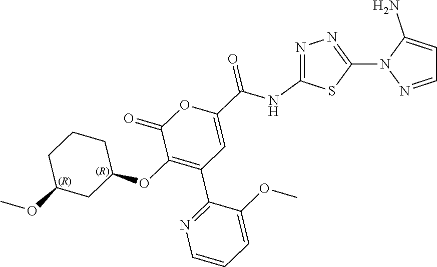 |
| | | N-(5-(5-amino-1H-pyrazol-1-yl)- |
| | | 1,3,4-thiadiazol-2-yl)-3-(((1R,3S)- |
| | | 3-methoxycyclohexyl)oxy)-4-(3- |
| | | methoxypyridin-2-yl)-2-oxo-2H- |
| | | pyran-6-carboxamide |
| — | — | — |

In some embodiments, the compound is Compound 6, Compound 7, Compound 8, Compound 9, Compound 10. Compound 17, Compound 22. Compound 24A*, Compound 24B*, Compound 40, Compound 43A*, Compound 43B*, Compound 44. Compound 45, Compound 46B, Compound 47B, Compound 55A*, Compound 55B*, Compound 62A*, Compound 62B*, Compound 63A*, Compound 63B*, Compound 71A*, Compound 71B*, or Compound 74, or a pharmaceutically acceptable salt of any of the foregoing.

### ii. Pharmaceutical Compositions

In some aspects, the present disclosure provides pharmaceutical compositions comprising a compound of Formula (I), or a pharmaceutically acceptable salt thereof, and a pharmaceutically acceptable carrier. Exemplary pharmaceutical acceptable carriers include excipients, diluents, and surfactants.

In some embodiments, the compounds of the present disclosure, or pharmaceutical compositions comprising same, can be administered in an amount effective to treat a disorder in a subject.

Administration can be accomplished via any mode of administration. Exemplary modes include systemic or local administration such as oral, nasal, parenteral, transdermal, subcutaneous, vaginal, buccal, rectal, or topical administration modes.

Depending on the intended mode of administration, the disclosed compounds and compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

Likewise, the disclosed compounds and compositions can also be administered by intravenous (both bolus and infusion), intraperitoneal, subcutaneous, or intramuscular in a form suitable for these types of administration. For example, parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Illustrative pharmaceutical compositions may be tablets or gelatin capsules comprising a compound of the present disclosure, or a pharmaceutically acceptable salt thereof, and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, or PEG200.

### iii. Methods of Treatment

In some aspects, the present disclosure provides a method of treating a disease or disorder disclosed herein in a subject in need thereof, comprising administering to the subject a compound of the present disclosure, or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject an effective amount of a compound of the present disclosure, or a pharmaceutically acceptable salt thereof, or a pharmaceutical composition of the present disclosure.

In some aspects, the present disclosure provides a method of modulating cGAS activity in a cell (e.g., in vitro or in vivo) comprising contacting the cell with an a compound of the present disclosure or a pharmaceutically acceptable salt thereof.

In some aspects, the present disclosure provides a method of modulating cGAS activity in a cell (e.g., in vitro or in vivo) comprising contacting the cell with an effective amount of a compound of the present disclosure or a pharmaceutically acceptable salt thereof.

In some embodiments, the disease or disorder is associated with implicated cGAS activity. In some embodiments, the disease or disorder is a disease or disorder in which cGAS activity is implicated.

In some aspects, the present disclosure provides a compound of the present disclosure, or a pharmaceutically acceptable salt thereof, for use in modulating cGAS activity (e.g., in vitro or in vivo).

In some aspects, the present disclosure provides a compound of the present disclosure, or a pharmaceutically acceptable salt thereof, for use in treating a disease or disorder disclosed herein.

In some aspects, the present disclosure provides use of a compound of the present disclosure, or a pharmaceutically acceptable salt thereof, in the manufacture of a medicament for modulating cGAS activity (e.g., in vitro or in vivo).

In some aspects, the present disclosure provides use of a compound of the present disclosure, or a pharmaceutically acceptable salt thereof, in the manufacture of a medicament for treating a disease or disorder disclosed herein.

In some aspects, the present disclosure provides use of a compound of the present disclosure, or a pharmaceutically acceptable salt thereof, in the manufacture of a medicament for treating a disease or disorder disclosed herein.

The present disclosure provides compounds that function as modulators of cGAS activity.

In some embodiments, modulation is inhibition.

In some embodiments, the disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an cGAS-related disease in a subject that has been determined to carry a germline or somatic non-silent mutation in cGAS.

In some aspects, the disease or disorder is cancer. In some embodiments, the cancer is bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, pancreatic cancer, penile cancer, testicular germ cell cancer, thymoma carcinoma, thymic carcinoma, lung cancer, ovarian cancer, or prostate cancer.

In some aspects, the disease or disorder is a central nervous system disorder. In certain embodiments, the central nervous system disorder is Parkinson's disease, Alzheimer's disease, traumatic brain injury, spinal cord injury, amyotrophic lateral sclerosis (ALS), multiple sclerosis, ataxia telangiectasia, or age-related macular degeneration.

In some aspects, the disease or disorder is kidney disease. In certain embodiments, the kidney disease is acute kidney disease, chronic kidney disease, or a rare kidney disease. In certain embodiments, the chronic kidney disease is diabetic nephropathy.

In some aspects, the disease or disorder is a skin disease. In certain embodiments, the skin disease is psoriasis, hidradenitis suppurativa (HS), or atopic dermatitis.

In some aspects, the disease or disorder is a rheumatic disease. In certain embodiments, the rheumatic disease is dermatomyositis, Still's disease, or juvenile idiopathic arthritis.

In some aspects, the disease or disorder is a liver disease. In certain embodiments, the liver disease is nonalcoholic steatohepatitis (NASH).

In some aspects, the disease or disorder is a cardiovascular disease. In certain embodiments, the cardiovascular disease is cardiomyopathy, atherosclerosis, or peripheral artery disease (PAD).

In some embodiments, the disease or disorder is a metabolic disease. In certain embodiments, the metabolic disease is obesity-induced insulin-resistance.

In some aspects, the disease or disorder is a cGAS-related disease in a subject that has been determined to carry a germline or somatic non-silent mutation in cGAS.

In some embodiments, the disease or disorder is an inflammatory, allergic or autoimmune disease such as systemic lupus erythematosus (SLE), cutaneous lupus erythematosus (CLE), Chilblain lupus, psoriasis, insulin-dependent diabetes mellitus (IDDM), scleroderma, Aicardi Goutières syndrome, dermatomyositis, systemic sclerosis, inflammatory bowel diseases, multiple sclerosis, rheumatoid arthritis, chronic kidney disease, or Sjogren's syndrome (SS).

In some embodiments, the disease or disorder is inflammation of any tissue or organ of the body, including musculoskeletal inflammation, vascular inflammation, neural inflammation, digestive system inflammation, ocular inflammation, inflammation of the reproductive system, and other inflammation.

In some embodiments, musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic). Ocular inflammation refers to inflammation of any structure of the eye, including the eye lids. Examples of ocular inflammation include blepharitis, blepharochalasis, conjunctivitis, dacryoadenitis, keratitis, keratoconjunctivitis sicca (dry eye), scleritis, trichiasis, and uveitis. Examples of inflammation of the nervous system include encephalitis, Guillain-Barre syndrome, meningitis, neuromyotonia, narcolepsy, multiple sclerosis, myelitis, and schizophrenia.

Examples of inflammation of the vasculature or lymphatic system include arthrosclerosis, arthritis, phlebitis, vasculitis, and lymphangitis.

Examples of inflammatory conditions of the digestive system include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system include cervicitis, chorioamnionitis, endometritis, epididymitis, omphalitis, oophoritis, orchitis, salpingitis, tubo-ovarian abscess, urethritis, vaginitis, vulvitis, and vulvodynia.

In some embodiments, the disease or disorder is an autoimmune conditions having an inflammatory component. Such conditions include systemic lupus erythematosus, cutaneous lupus erythematosus, acute disseminated alopecia universalise, Bechet's disease. Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis. Goodpasture's syndrome. Grave's disease, Guillain-Barre syndrome, Hashimoto's disease. Henoch-Schonlein purpura, Kawasaki's disease, microscopic colitis, microscopic polyarteritis, mixed connective tissue disease, multiple sclerosis, myasthenia gravis, opsoclonus myoclonus syndrome, optic neuritis. Ord's thyroiditis, pemphigus, polyarteritis nodosa, polymyalgia, rheumatoid arthritis, Reiter's syndrome, Sjogren's syndrome, Aicardi Goutires syndrome, temporal arteritis. Wegener's granulomatosis, warm autoimmune haemolytic anemia, interstitial cystitis. Lyme disease, morphea, psoriasis, sarcoidosis, scleroderma, ulcerative colitis, and vitiligo.

In some embodiments, the disease or disorder is a T-cell mediated hypersensitivity diseases having an inflammatory component. Such conditions include contact hypersensitivity, contact dermatitis (including that due to poison ivy), urticaria, skin allergies, respiratory allergies (hay fever, allergic rhinitis), and gluten-sensitive enteropathy (Celiac disease).

In some embodiments, other inflammatory conditions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome. Sezary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, leukemia and lymphomas in adults, acute leukemia of childhood, regional enteritis, autoimmune vasculitis, multiple sclerosis, chronic obstructive pulmonary disease, solid organ transplant rejection, and sepsis.

### iv. Methods of Preparation

Compounds of Formula (I) may be synthesized following General Schemes 1-8, as provided below. The Examples further described non-limiting examples of this general syntheses.

For example, as depicted in General Scheme 1, 3-hydroxy-2-oxo-2H-pyran-6-carboxylic acid of Formula (A), or salt thereof, may be protected as the alkyl ester of Formula (B), or salt thereof, wherein R^{a} is C_{1 -6} alkyl or C₁₋₆ haloalkyl, followed by halogenation at the C4 position to provide a compound of Formula (C), or salt thereof, wherein X is Cl, Br, or I. Hydroxyl protection with group R¹, as defined herein, may provide a compound of Formula (D), or salt thereof. Deprotection of the alkyl ester of Formula (D), or salt thereof, may provide a carboxylic acid compound of Formula (D), or salt thereof, wherein R^{a} is hydrogen.

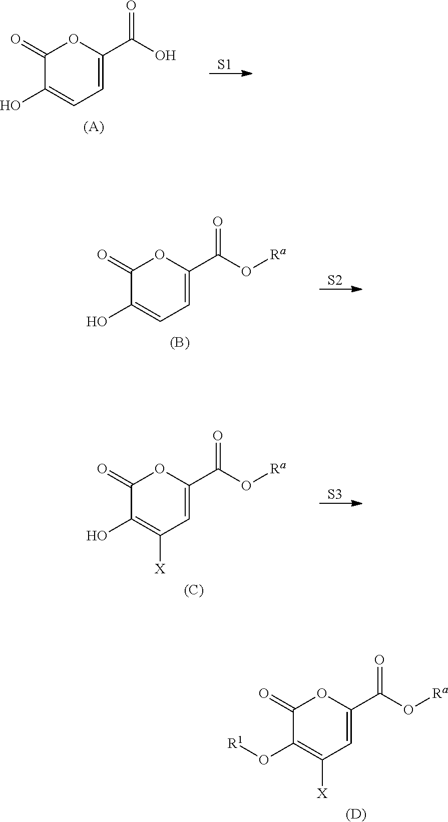

As depicted in General Scheme 2, reacting a hydrazine carbothioamide of Formula (G), or salt thereof, with a carboxylic acid containing compound of Formula (F-1), or salt thereof, or nitrile containing compound of Formula (F-2), or salt thereof, wherein R⁴ and m are as defined herein, may provide a 1,3,4-thiadiazol-2-amine of Formula (H-1), or salt thereof. Alternatively, as depicted in General Scheme 3, amine compounds of Formula (H-2), wherein the nitrogen atom of the heteroaryl Ring A is directly linked to the thiadiazole moiety and wherein R⁴ and m are as defined herein, may be prepared by coupling a 5-halo-1,3,4-thiadiazol-2-amine of Formula (M), or salt thereof, wherein Y is Cl, Br, or 1, with an amine of Formula (L), or salt thereof.

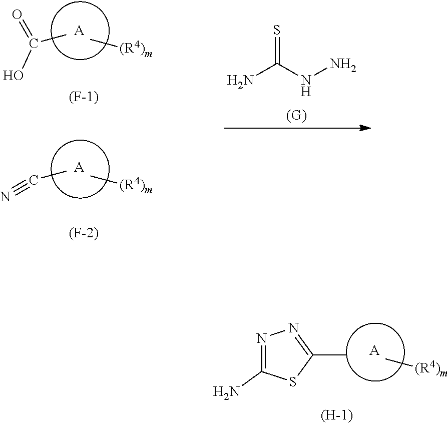

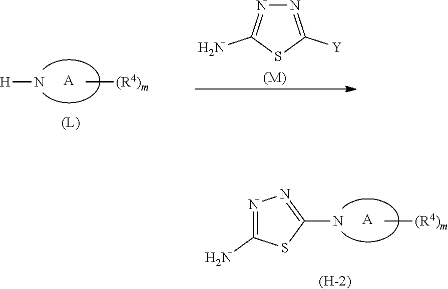

As depicted in General Scheme 4, cross-coupling of an aryl or heteroaryl boronic acid or boronic ester (K-1), or an aryl or heteroaryl stannane (K-2), or an aryl or heteroaryl sulfonate (K-3) with an alkyl ester of Formula (D), or salts thereof, wherein R^{a} is C₁₋₆ alkyl or C₁₋₆ haloalkyl, R^{C} is Ring C, R is H, an optionally substituted C₁₋₆ alkyl, or two R groups are joined to form a 5-6 membered ring, and each R′ is independently an optionally substituted C₁₋₆alkyl (wherein optional substitution includes, but is not limited to, —CO₂CH₃ groups) may provide a compound of Formula (N), or salt thereof. The compound of Formula (N), or salt thereof, may then be deprotected to provide a carboxylic acid compound of Formula (N), or salt thereof, wherein R^{a} is hydrogen.

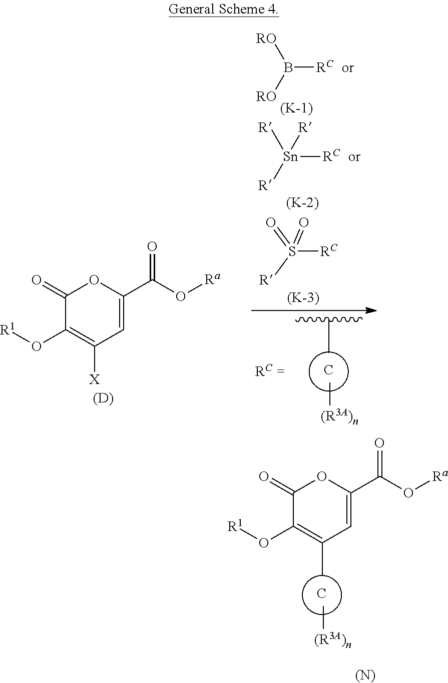

The above-described compounds of Formula (D) and (N), or salts thereof, wherein R^{a} is hydrogen, C₁₋₆ alkyl or C₁₋₆ haloalkyl, and amine compounds of Formula (H-1) or (H-2), or salts thereof, each may be used as intermediates in preparing compounds of Formula (I), or salts thereof.

For example, as depicted in General Scheme 5, peptide coupling the amine of Formula (H-1), or salt thereof, with the compound of Formula (D), or salt thereof, wherein R^{a} is hydrogen, C₁₋₆ alkyl or C₁₋₆ haloalkyl, may provide an amide compound of Formula (J-1), or salt thereof. The amide compound of Formula (J-1), or salt thereof, (also referred to herein as a “halo-pyrone reagent”), may then be cross-coupled with an aryl or heteroaryl boronic acid or boronic ester (K-1), or salt thereof, or an aryl or heteroaryl stannane (K-2), or salt thereof, or an aryl or heteroaryl sulfonate (K-3), or salt thereof, (also referred to herein as an “aryl boron reagent” or “aryl tin reagent” or “aryl sulfonate reagent”).

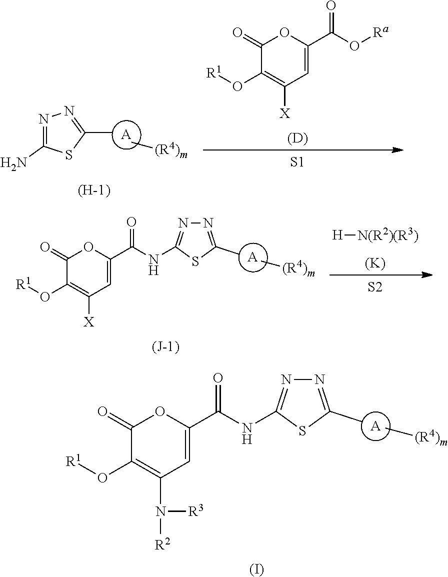

Alternatively, as depicted in General Scheme 6, peptide coupling of the amine of Formula (H-2), or salt thereof, with the compound of Formula (D), or salt thereof, wherein R^{a} is hydrogen. C₁₋₆ alkyl or C₁₋₆ haloalkyl, may provide an amide compound of Formula (J-2), or salt thereof. The amide compound of Formula (J-2), or salt thereof (also referred to herein as a “halo-pyrone reagent”), may then be cross-coupled with aryl or heteroaryl boronic acid or boronic ester (K-1), or salt thereof, or an aryl or heteroaryl stannane (K-2), or salt thereof, or an aryl or heteroaryl sulfonate (K-3), or salt thereof, (also referred to herein as an “aryl boron reagent” or “aryl tin reagent” or “aryl sulfonate reagent”), to provide a compound of Formula (I′), or salt thereof, wherein the nitrogen atom of the heteroaryl Ring A is directly linked to the thiadiazole moiety.

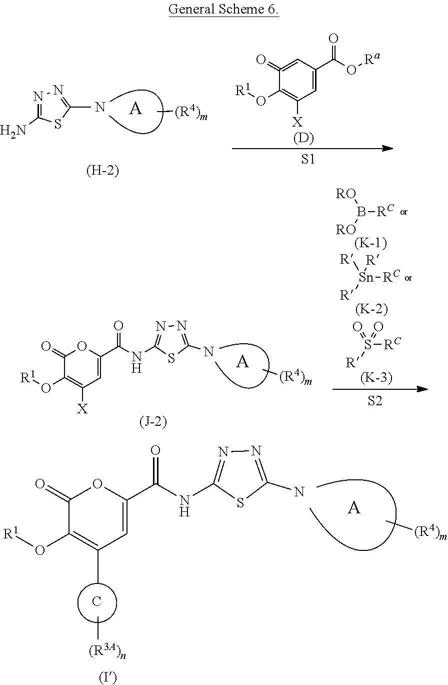

In other embodiments, such as depicted in General Scheme 7, peptide coupling of the amine of Formula (H-1), or salt thereof, (also referred to herein as an “ADT amine reagent”), with a compound of Formula (N), or salt thereof, (also referred to herein as an “aryl-pyrone reagent”), wherein R^{a} is hydrogen, C₁₋₆ alkyl or C₁₋₆ haloalkyl, may provide a compound of Formula (I), or salt thereof.

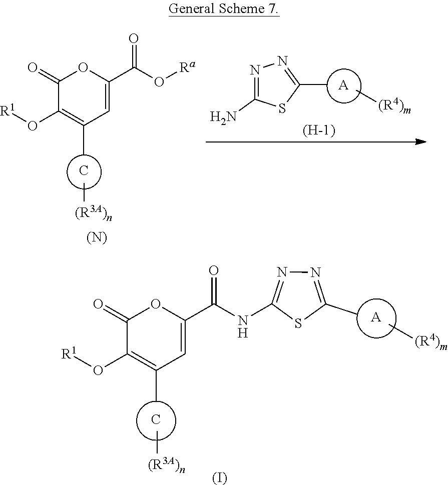

In yet other embodiments, such as depicted in General Scheme 8, peptide coupling of the amine of Formula (H-2), or salt thereof. (also referred to herein as an “ADT amine reagent”), with a compound of Formula (N), or salt thereof, wherein R^{a} is hydrogen, C₁₋₆ alkyl or C₁₋₆ haloalkyl. (also referred to herein as an “aryl-pyrone reagent”), may provide a compound of Formula (I′), or salt thereof, wherein the nitrogen atom of the heteroaryl Ring A is directly linked to the thiadiazole moiety.

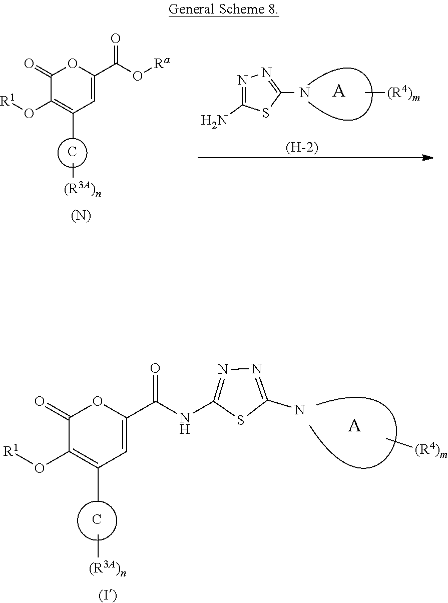

### v. Biological Assays

Compounds designed, selected and/or optimized by methods described above, once produced, can be characterized using a variety of assays known to those skilled in the art to determine whether the compounds have biological activity. For example, the compounds described herein can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the compounds described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, as well as assays for determining hcGAS potency and inhibitory activity, unbound clearance, solubility, and permeability.

In some embodiments, the compounds of the instant disclosure may be tested for their human-cGAS (h-cGAS) inhibitory activity using known procedures, such as the methodology reported in Lama et al., Nature Communications (2019) 10:2261 (2019). See also Examples, Assay Methods section.

In some embodiments, the compounds of the instant disclosure may be tested for unbound clearance (Clu) following known procedures, such as described in Miller et al., J. Med. Chem. (2020) 63:12156-12170. For example, unbound clearance (Clu) may be calculated by dividing total clearance (‘CL’ in mLUmin/kg) as measured in blood or plasma by the unbound fraction in plasma (fu).

In some embodiments, the solubility of compounds of the instant disclosure may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media may be measured using serial dilution and two hour incubation period, followed by filtration, and reported in uM by LC-MS/MS. Thermodynamic solubility in physiologically relevant media may be measured by LC-MS/MS, after a twenty-four hour incubation, followed by filtration, and reported in mg/mL.

In some embodiments, the permeability of compounds of the instant disclosure may be determined following known procedures, such as described in Wang et al. J Mass Spectrom. (2000) 35:71-76. For example, permeability across cell membranes may be measured using either Caco-2 or MDCK-MDR1 cell lines in Transwell plates, after measuring the compound in both apical and basolateral chambers, and reported as an apparent permeability Papp A-B in 106 cm/s.

### vi. Additional Embodiments

Additional embodiments are provided according to the following numbered Embodiments.
Embodiment 1. A compound of Formula (I):

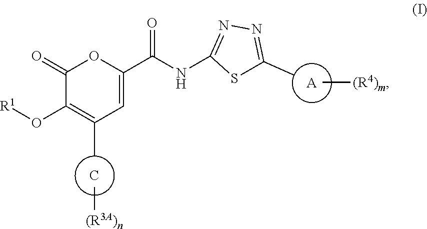

or a pharmaceutically acceptable salt thereof, wherein:
   Ring A is a 5-membered monocyclic heteroaryl;
   R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen. —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄ alkyl, or C₁-C₄ haloalkyl, wherein the alkyl and haloalkyl are independently substituted with 0, 1, 2, 3, or 4 R^{1D}; each R^{1C} is independently hydrogen, C₁-C₃ alkyl, C₁-C₃ haloalkyl, or —OR^{1F}; each R^{1D} is independently halogen, —OR^{1F}, or —N(R^{1F})₂; and each R^{1F} is independently hydrogen, C₁-C₃ alkyl, or C₁-C₃ haloalkyl;
   L₃ is a bond, C₁-C₃ alkylene, or —(C₁-C₃ alkylene)-O—, wherein the alkylene is independently substituted with 0, 1, 2, 3, or 4 R^{1E}; and each R_{1E} is independently —(C₁-C₃ alkylene)-OR^{1B} or —OR^{1B}, or two instances of R^{1E} are taken together to form ═O;
   Ring C is a C₆-C₁₀ aryl or 5- to 10-membered heteroaryl;
   each R^{3A} is independently C₁-C₃ alkyl, C₂-C₃ alkenyl, C₂-C₃ alkynyl, halogen, -L₁-CN, -L₁-SOR^{3C}, -L₁-SO₂R^{3B}, -L₁-SR^{3B}, -L₁-PO(R^{3C})₂, -L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
   each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R¹ is independently C₁-C₃ alkyl or C₁-C₃ haloalkyl;
each R^{1D} is independently halogen. —OR^{3E}, —CN, C₁-C₃ alkyl, or C₁-C₃ haloalkyl; each R^{3E} is independently hydrogen, C₁-C₄ alkyl, or C₁-C₄haloalkyl;
each R⁴ is independently halogen, —CN, -L₂-OR⁴-L₂-N(R^{4B})₂, C₁-C₆ alkyl, or C₁-C₃ haloalkyl;
each R^{4A} and R^{4B} are independently hydrogen, C₁-C₃ alkyl, C₁-C₃ haloalkyl, or —C(═O)R^{4C}, wherein R^{4C} is C₁-C₆ alkyl or C₁-C₆ haloalkyl;
each L₁ and L₂ is independently a bond, C₁-C₃ alkylene, or C₁-C₃ haloalkylene;
m is 0, 1, or 2; and
n is 0, 1, 2, 3, or 4.
Embodiment 2. The compound of Embodiment 1, or a pharmaceutically acceptable salt thereof, wherein the compound is of Formula (I′):

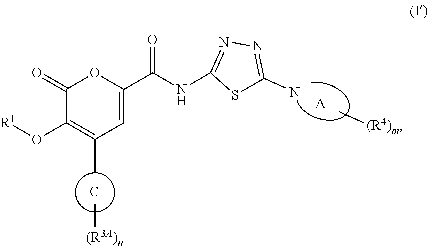

or a pharmaceutically acceptable salt thereof, wherein a nitrogen atom of the heteroaryl Ring A is directly linked to the thiadiazole moiety.
Embodiment 3. The compound of Embodiment 1 or 2, or a pharmaceutically acceptable salt thereof, wherein Ring C is phenyl substituted with 0, 1, 2, 3, or 4 R^{3A}.
Embodiment 4. The compound of Embodiment 1 or 2, or a pharmaceutically acceptable salt thereof, wherein Ring C is a 6-membered heteroaryl, wherein the heteroaryl has 1 ring N atom.
Embodiment 5. The compound of Embodiment 1 or 2, or a pharmaceutically acceptable salt thereof, wherein Ring C is a 5-membered heteroaryl, wherein the heteroaryl has 2 ring N atoms.
Embodiment 6. The compound of any one of the preceding Embodiments, or a pharmaceutically acceptable salt thereof, wherein Ring C is:

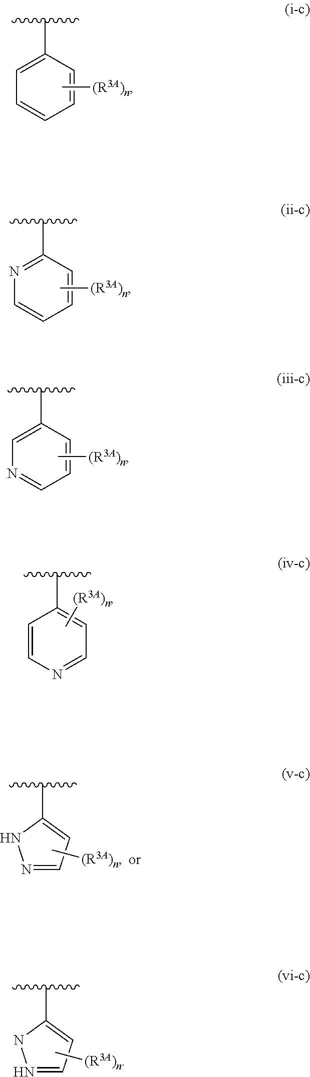

Embodiment 7. The compound of any one of the preceding Embodiments, wherein the PGP compound is of Formula (I-d):

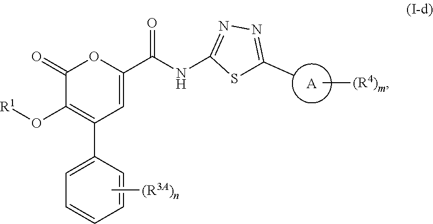

or a pharmaceutically acceptable salt thereof.
Embodiment 8. The compound of any one of the preceding Embodiments, wherein the compound is of Formula (I-e-1), (I-e-2), or (I-e-3):

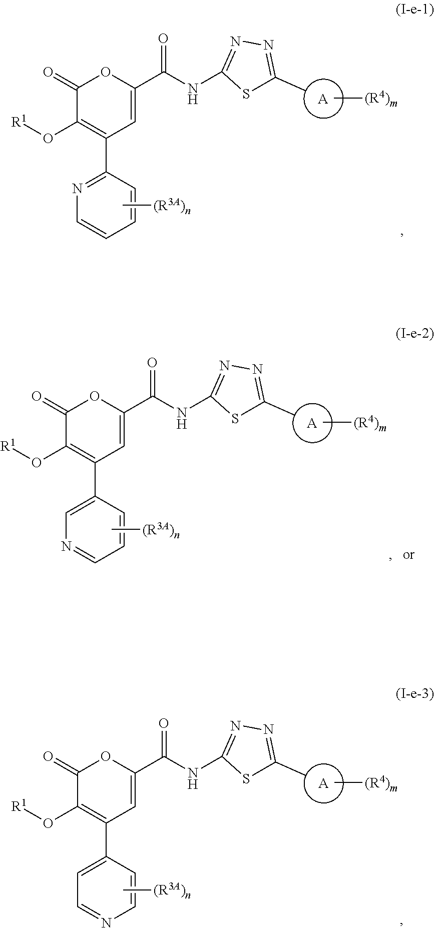

or a pharmaceutically acceptable salt thereof.
Embodiment 9. The compound of any one of the preceding Embodiments, wherein the compound is of Formula (I-f-1) or (I-f-2):

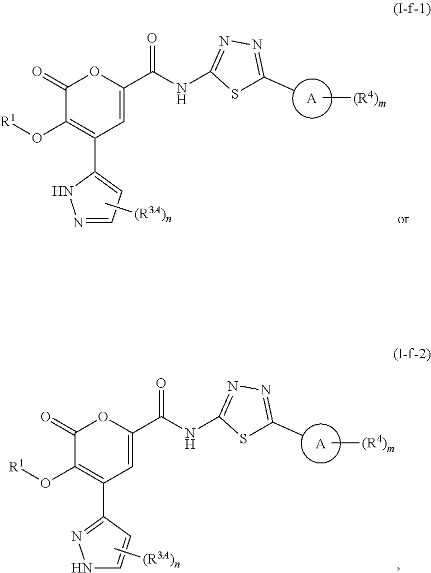

or a pharmaceutically acceptable salt thereof.
Embodiment 10. The compound of any one of the preceding Embodiments, or a pharmaceutically acceptable salt thereof, wherein at least one R^{3A} is independently —CN, —OCH₃, —OCHF₂, —CH₃, or Cl.
Embodiment 11. The compound of any one of the preceding Embodiments, or a pharmaceutically acceptable salt thereof, wherein Ring C is:

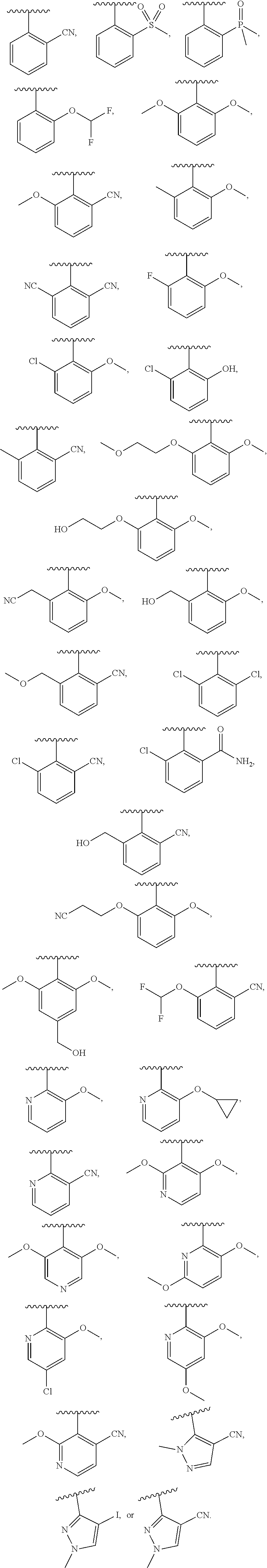

Embodiment 12. The compound of any one of the preceding Embodiments, or a pharmaceutically acceptable salt thereof, wherein Ring C is:

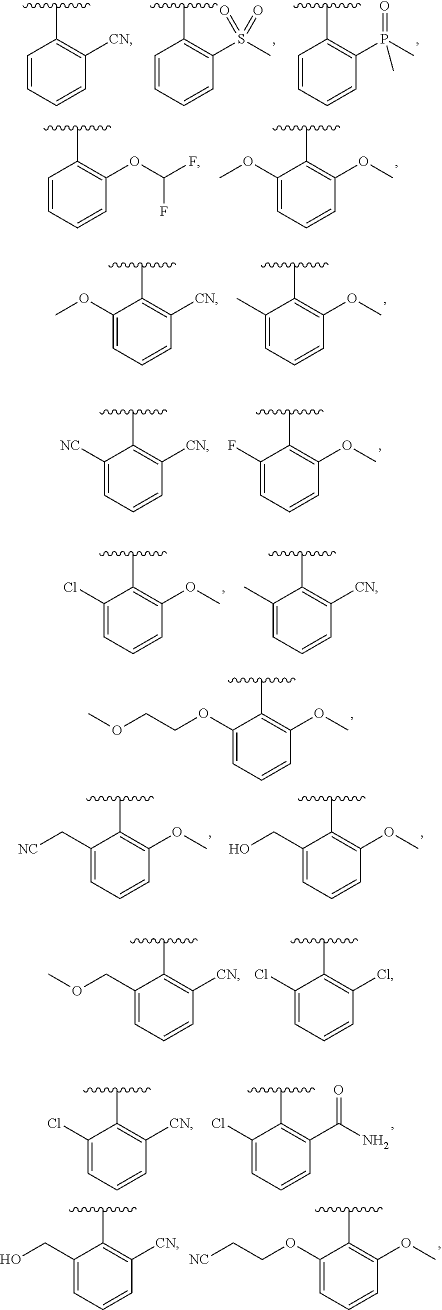

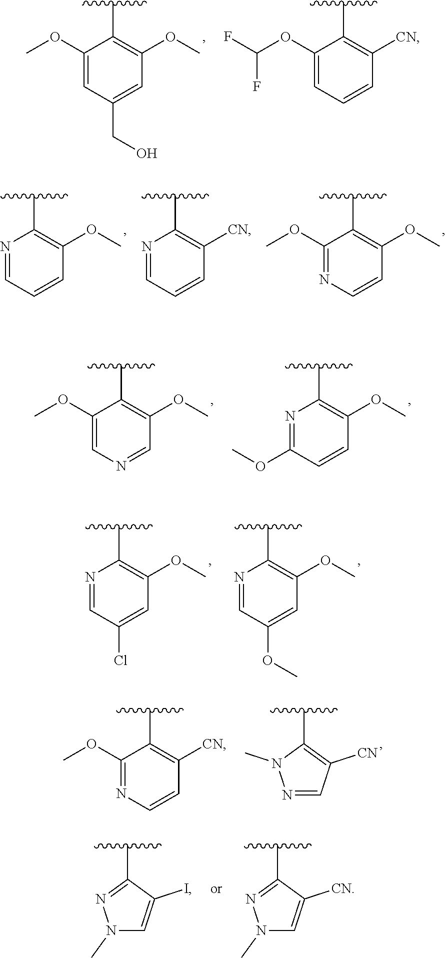

Embodiment 13. The compound of any one of the preceding Embodiments, or a pharmaceutically acceptable salt thereof, wherein R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}.
Embodiment 14. The compound of any one of the preceding Embodiments, or a pharmaceutically acceptable salt thereof, wherein R¹ is -L₃-(C₃-C₆ carbocyclyl), wherein the carbocyclyl is substituted with 1 R^{1A}; R^{1A} is —OR^{1B}; R^{1B} is hydrogen; and L₃ is bond or —(C₁-C₃ alkylene)-O—.
Embodiment 15. The compound of any one of the preceding Embodiments, or a pharmaceutically acceptable salt thereof, wherein R¹ is -L₃-(4- to 10-membered heterocyclyl), wherein the heterocyclyl is substituted with 0 R^{1A}; and L₃ is C₁-C₃ alkylene substituted with 0 R^{1E}.
Embodiment 16. The compound of any one of the preceding Embodiments, or a pharmaceutically acceptable salt thereof, wherein R¹ is:

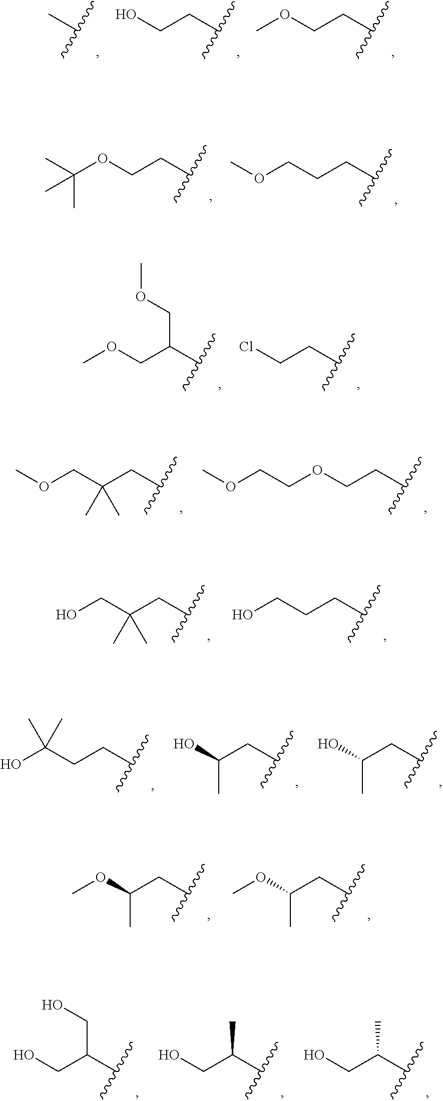

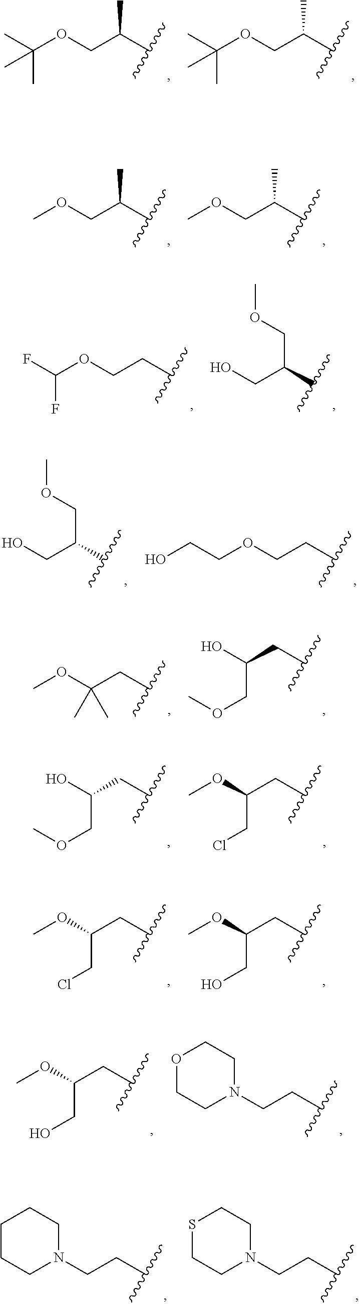

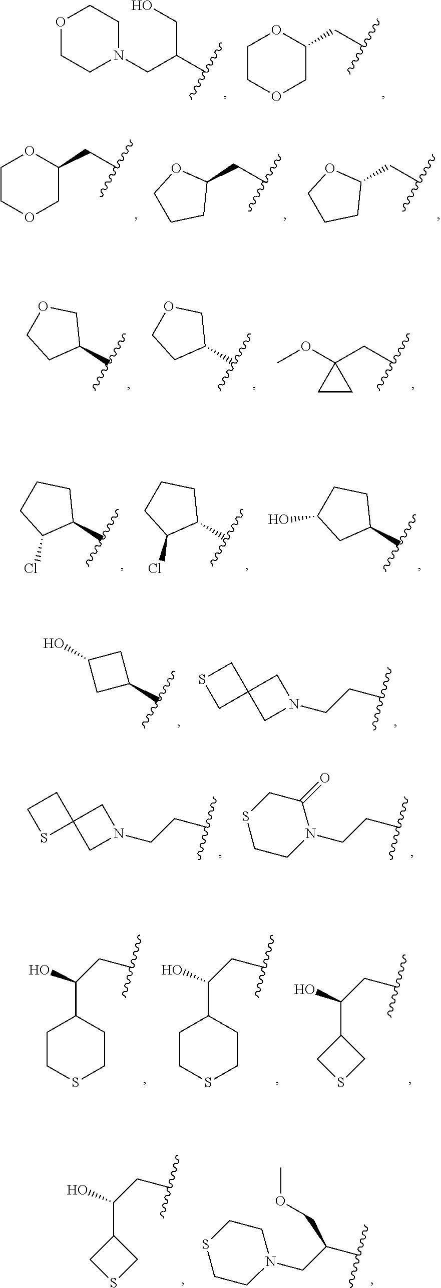

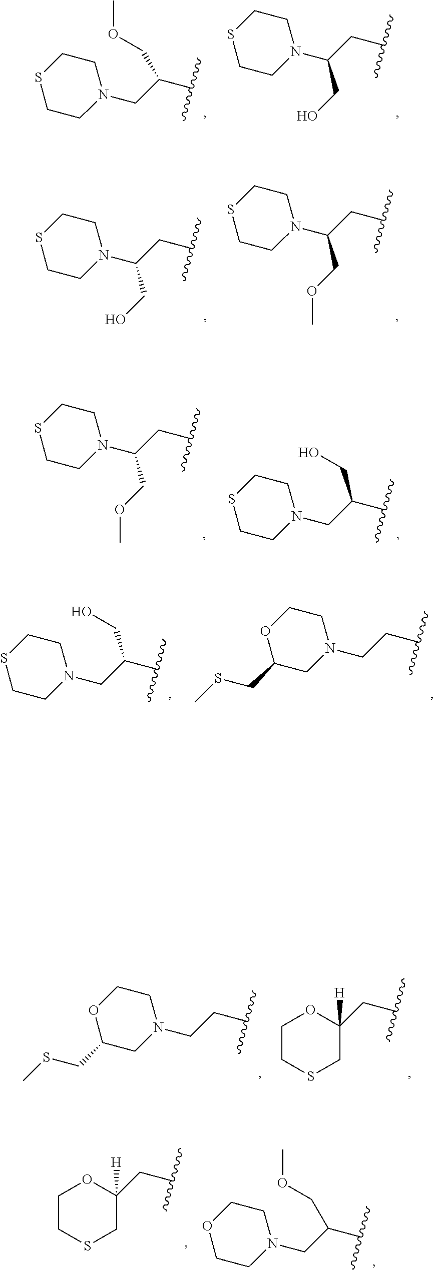

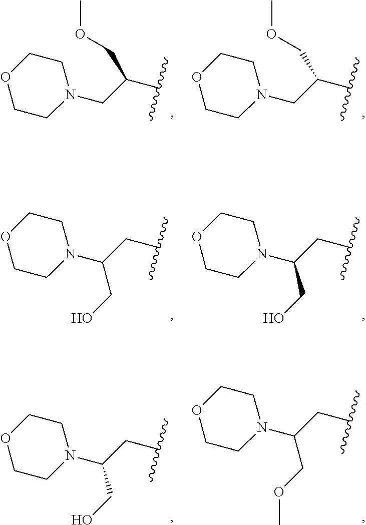

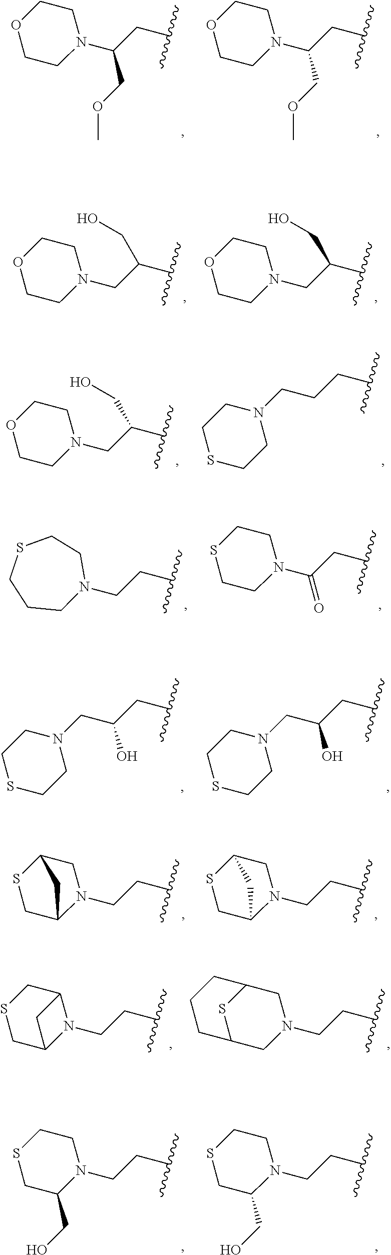

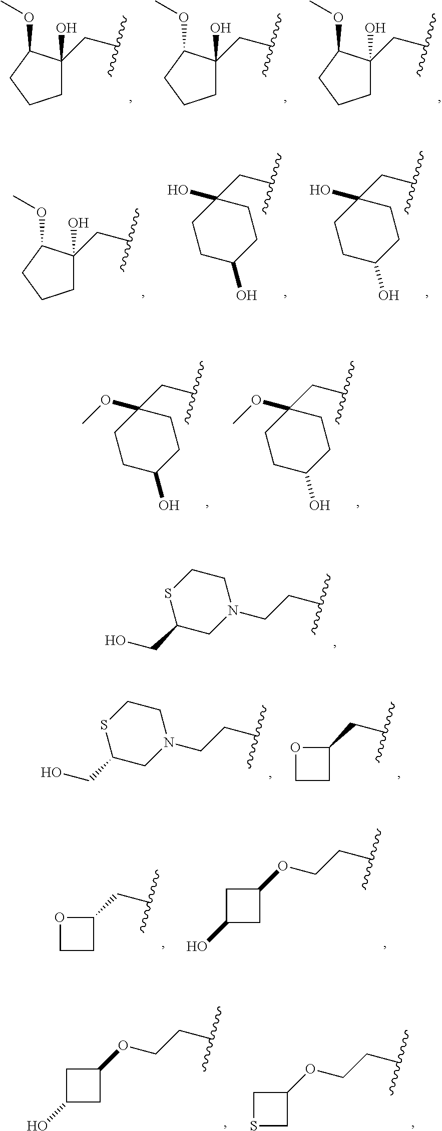

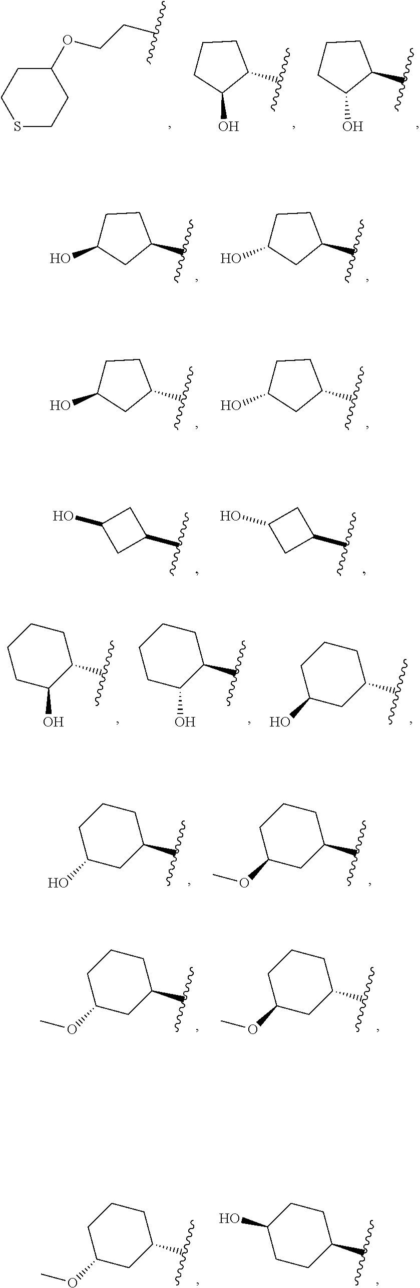

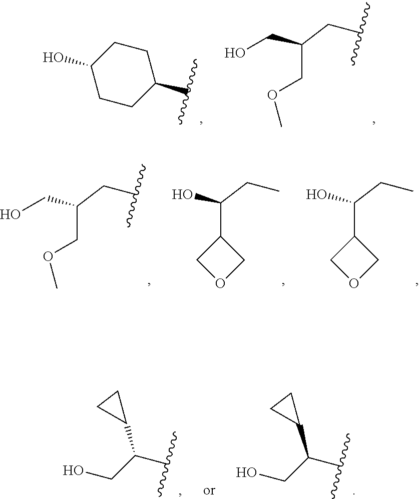

Embodiment 17. The compound of any one of the preceding Embodiments, or a pharmaceutically acceptable salt thereof, wherein R¹ is:

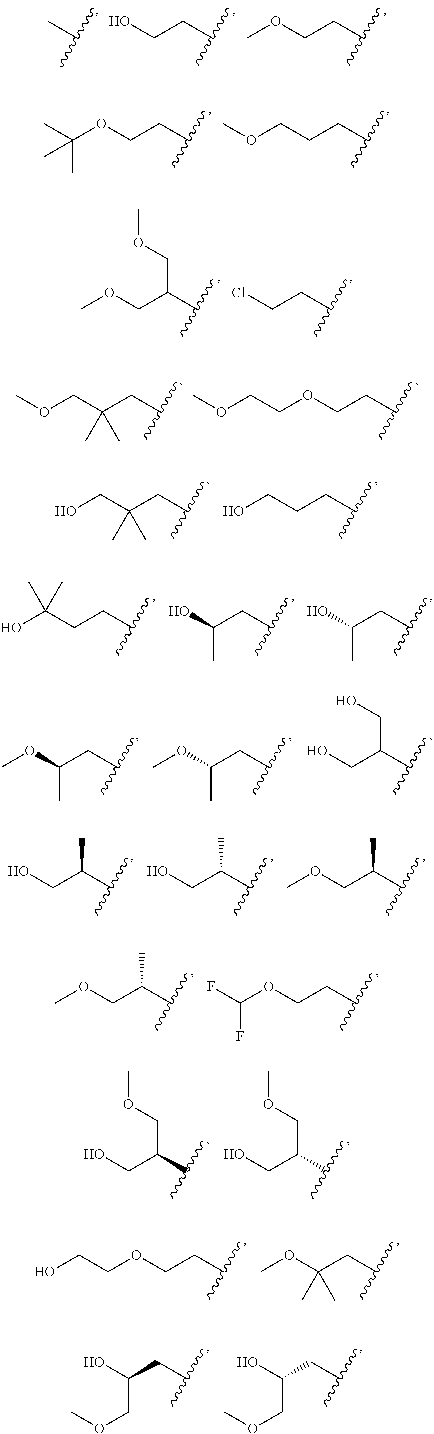

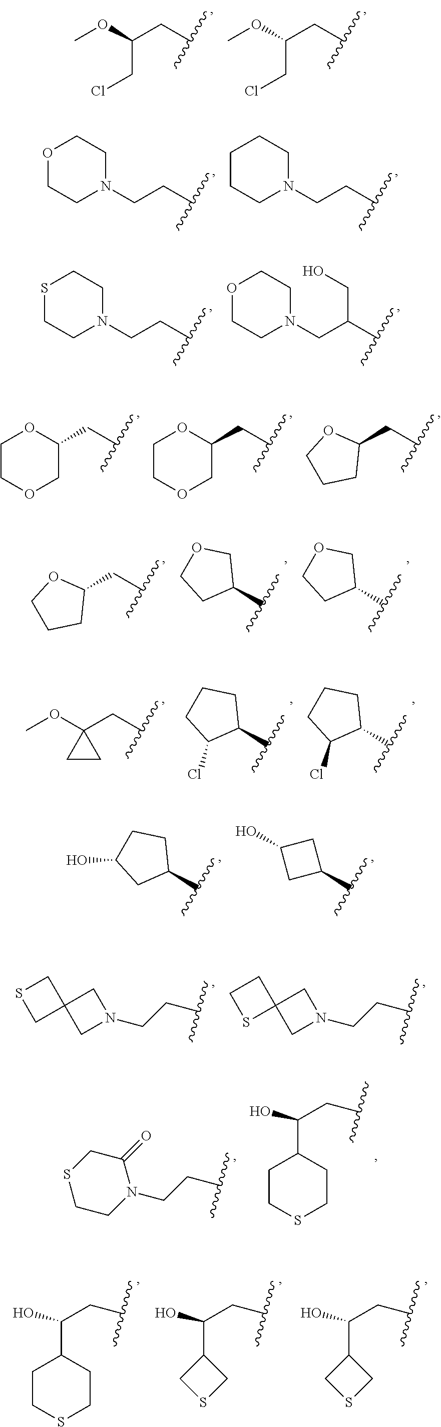

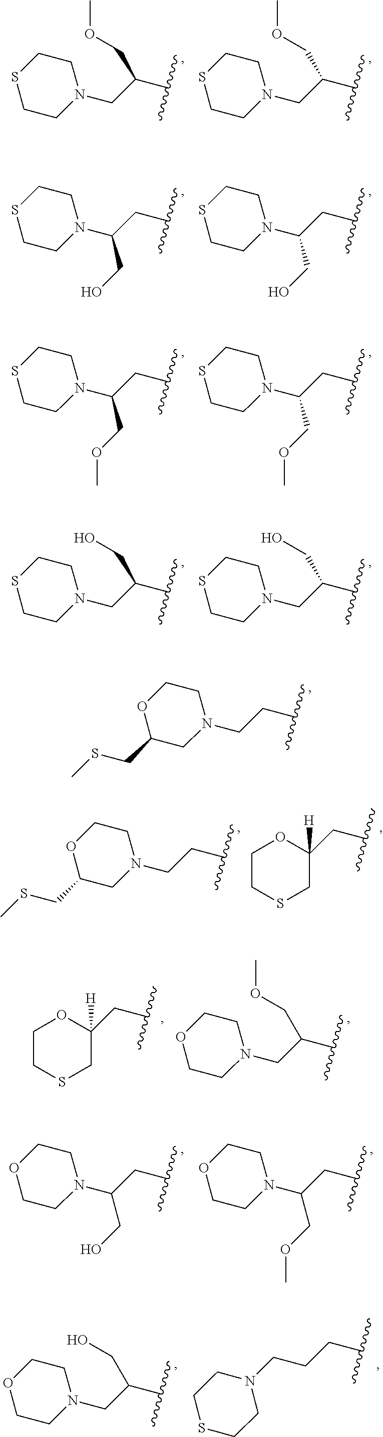

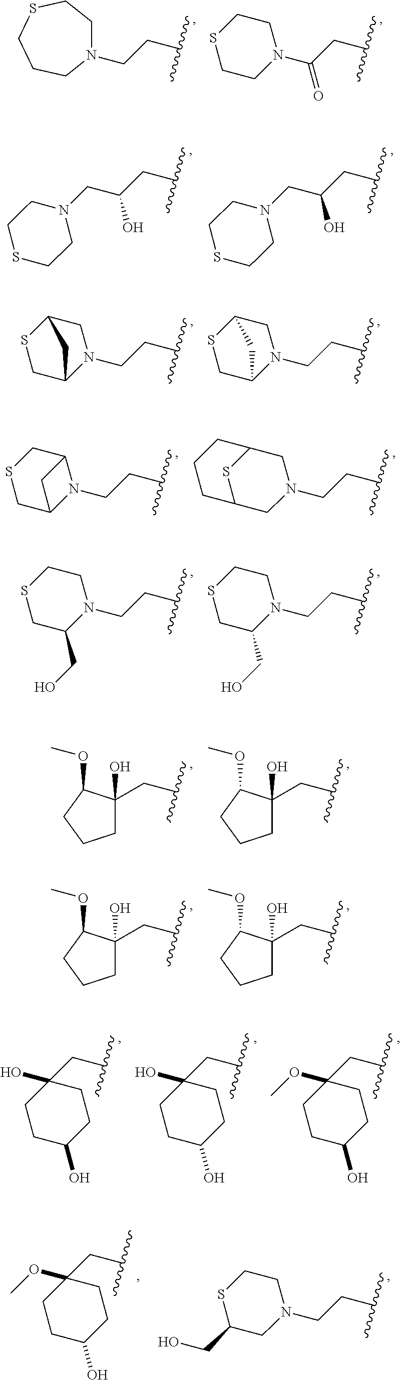

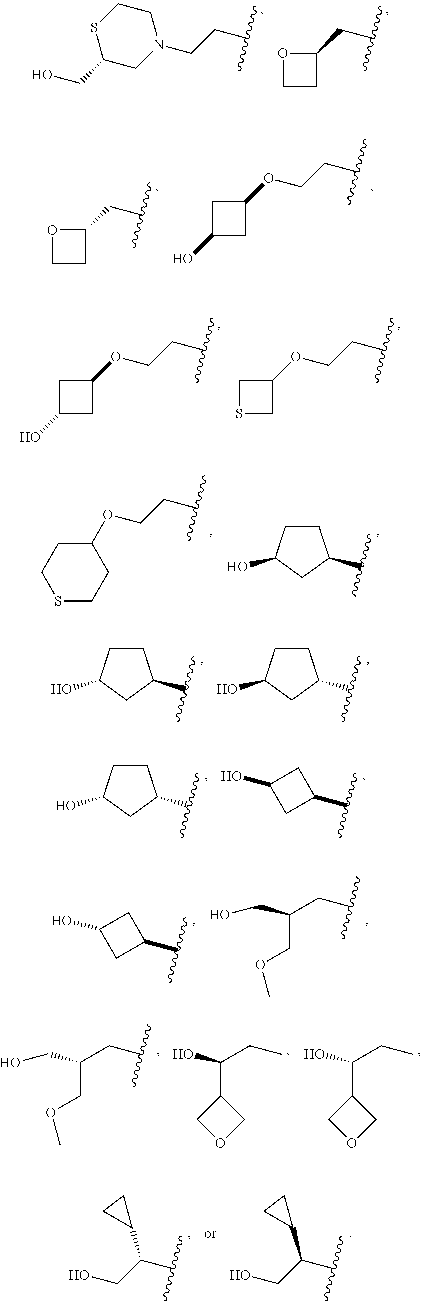

Embodiment 18. The compound of any one of the preceding Embodiments, or a pharmaceutically acceptable salt thereof, wherein Ring A is:

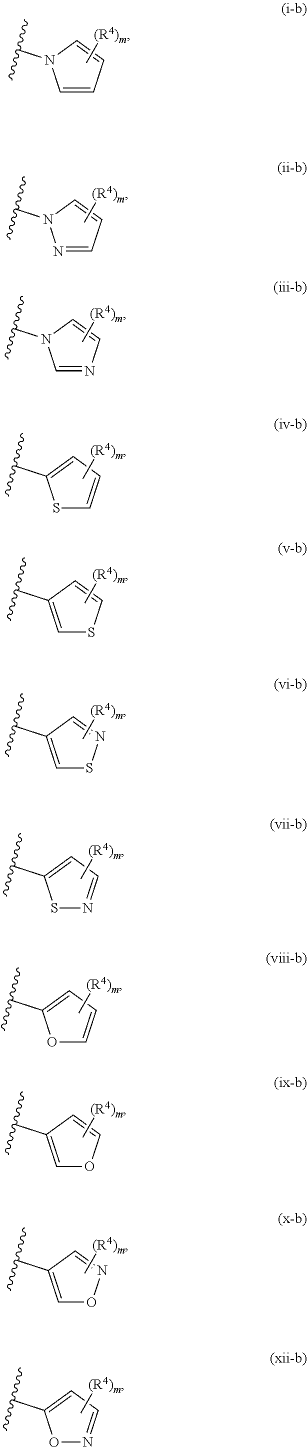

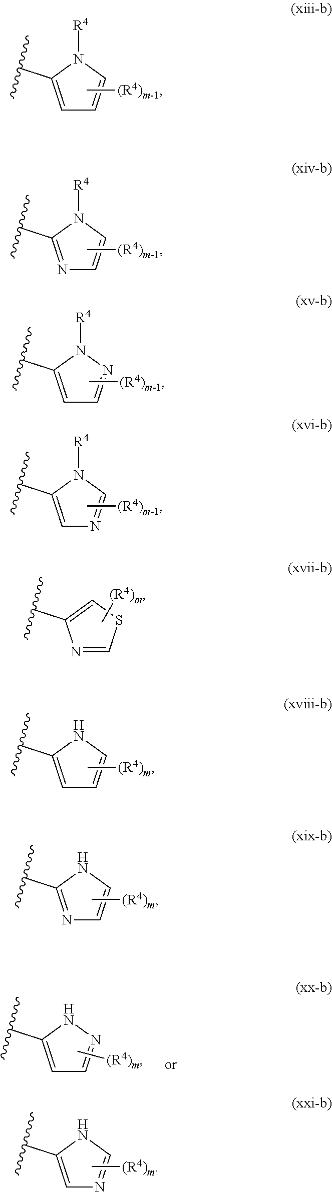

Embodiment 19. The compound of any one of the preceding Embodiments, or a pharmaceutically acceptable salt thereof, wherein Ring A is:

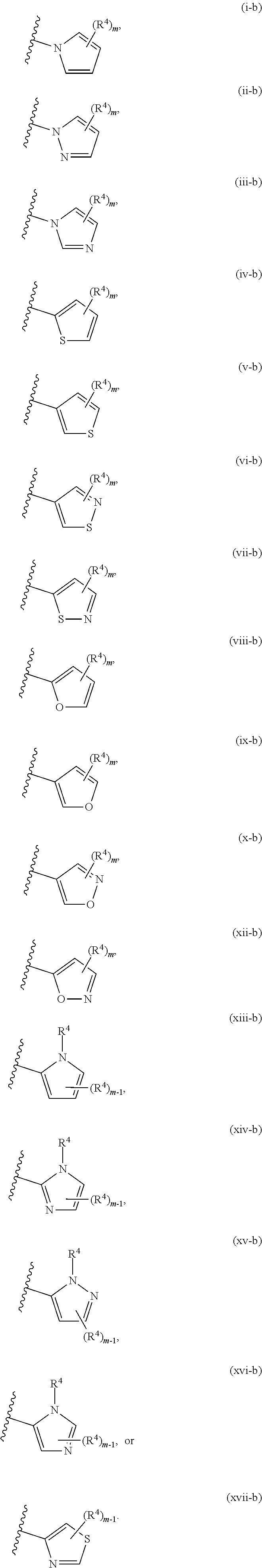

Embodiment 20. The compound of any one of the preceding Embodiments, or a pharmaceutically acceptable salt thereof, wherein Ring A is one of the following formulae:

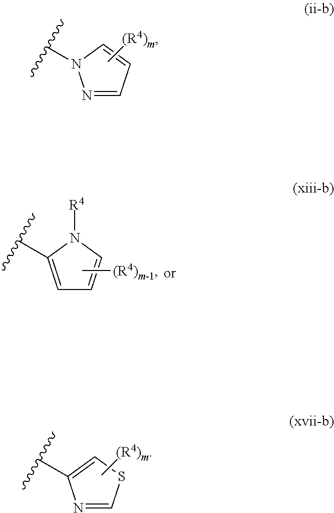

Embodiment 21. The compound of any one of the preceding Embodiments, or a pharmaceutically acceptable salt thereof, wherein each R⁴ is independently halogen, —CN, -L₂-OR^{4A}, -L₂-N(R^{4B})₂, C₁-C₆ alkyl, or C₁-C₆ haloalkyl.
Embodiment 22. The compound of any one of the preceding Embodiments, or a pharmaceutically acceptable salt thereof, wherein each R⁴ is independently —CH₃ or —NH₂.
Embodiment 23. The compound of any one of the preceding Embodiments, or a pharmaceutically acceptable salt thereof, wherein Ring A is:

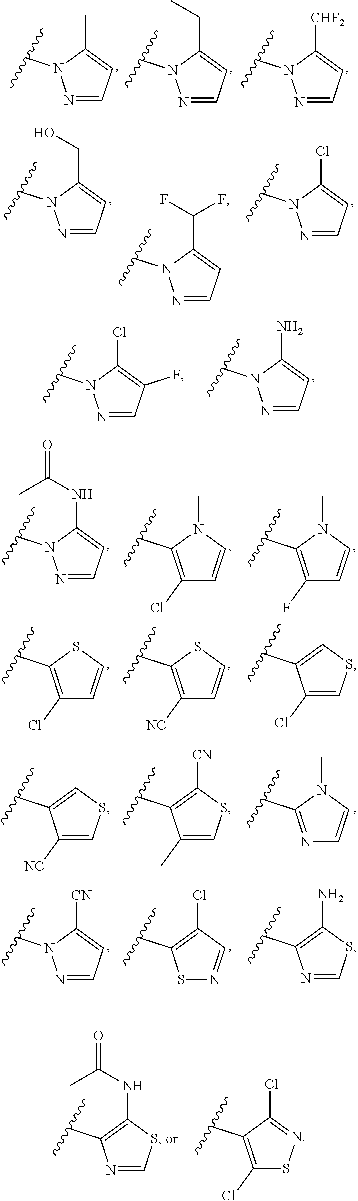

Embodiment 24. The compound of any one of the preceding Embodiments, or a pharmaceutically acceptable salt thereof, wherein Ring A is:

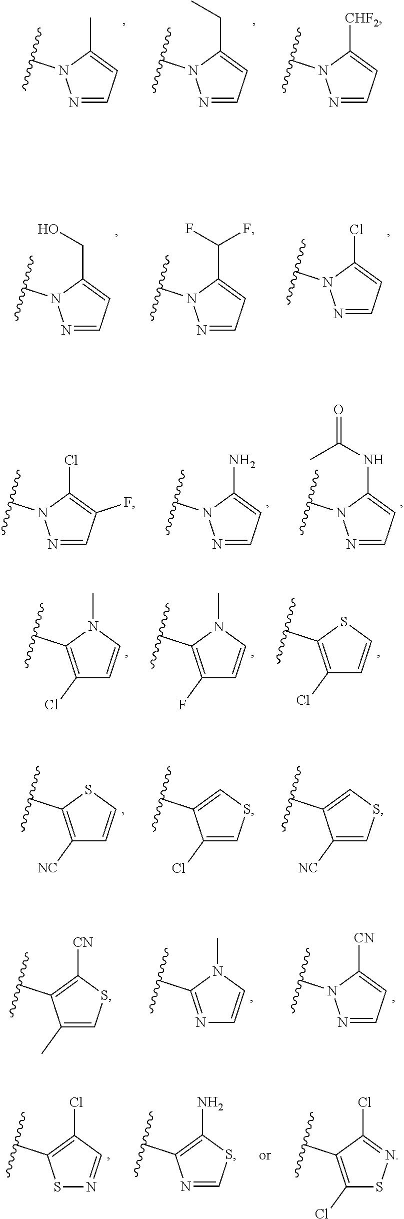

Embodiment 25. The compound of any one of the preceding Embodiments, or a pharmaceutically acceptable salt thereof, wherein Ring A is:

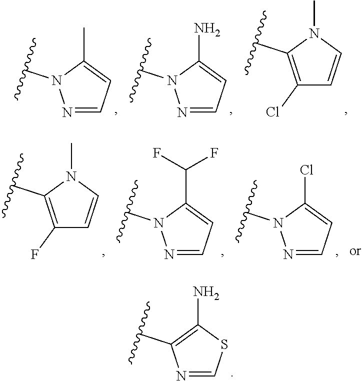

Embodiment 26. The compound of any one of the preceding Embodiments, or a pharmaceutically acceptable salt thereof, wherein m is 1.
Embodiment 27. The compound of any one of the preceding Embodiments, or a pharmaceutically acceptable salt thereof, wherein m is 2.
Embodiment 28. The compound of any one of the preceding Embodiments, or a pharmaceutically acceptable salt thereof, wherein n is 1.
Embodiment 29. The compound of any one of the preceding Embodiments, or a pharmaceutically acceptable salt thereof, wherein n is 2.
Embodiment 30. The compound of Embodiment 1, wherein the compound is selected from those in Table 1 or Table 2, and pharmaceutically acceptable salts thereof.
Embodiment 31. A pharmaceutical composition comprising the compound of any one of the preceding Embodiments, or a pharmaceutically acceptable salt thereof, and a pharmaceutically acceptable carrier.
Embodiment 32. A method of treating a disease or disorder in a subject in need thereof comprising administering to the subject a compound of any one of the preceding Embodiments, or a pharmaceutically acceptable salt thereof, or a pharmaceutical composition thereof.
Embodiment 33. A method of modulating cGAS activity in a cell comprising contacting the cell with a compound of any one of the preceding Embodiments, or a pharmaceutically acceptable salt thereof.
Embodiment 34. A method of preparing a compound of Formula (I):

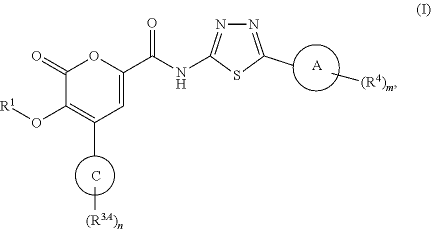

or a salt thereof, wherein Ring A, Ring C, R¹, R^{3A}, R⁴, m, and n are defined in Embodiment 1, the method comprising peptide coupling of a compound of Formula (H-1), or salt thereof, with a compound of Formula (N), or salt thereof:

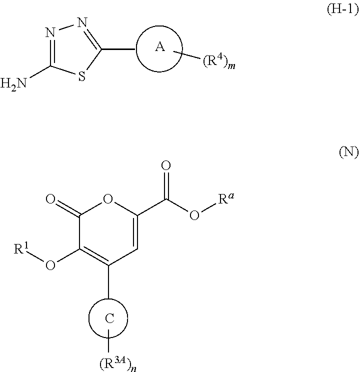

wherein R^{a} is hydrogen, C₁₋₆ alkyl, or C₁₋₆ haloalkyl, to provide a compound of Formula (I), or salt thereof.
Embodiment 35. The method of Embodiment 34, wherein the compound of Formula (H-1), or salt thereof, is of Formula (H-2):

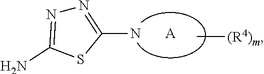

or salt thereof,
and wherein the method provides a compound of Formula (I′):

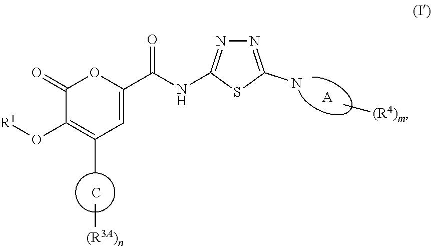

or salt thereof.
Embodiment 36. The method of Embodiment 34 or 35, further comprising cross-coupling a compound of Formula (K-1), (K-2), or (K-3), or salt thereof, with a compound of Formula (D), or salt thereof,

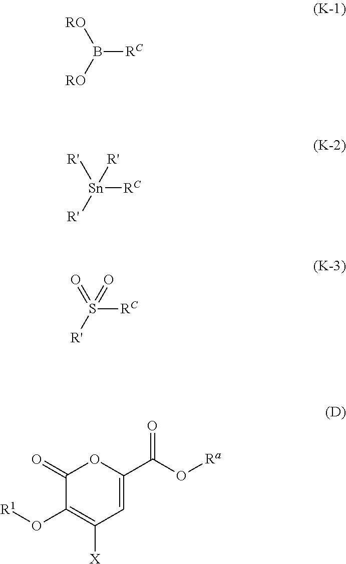

wherein:
   R^{a} is C₁₋₆ alkyl or C₁₋₆ haloalkyl;
   R^{C} is Ring C;
   X is Cl, Br, or I;
   each R is independently H, an optionally substituted C₁₋₆ alkyl, or two R groups are joined to form a 5-6 membered ring; and
   each R′ is independently an optionally substituted C₁₋₆ alkyl;
to provide a compound of Formula (N), or salt thereof.
Embodiment 37. A method of preparing a compound of Formula (I):

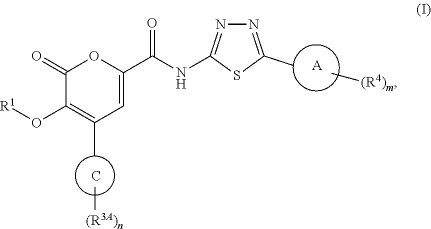

or a salt thereof, wherein Ring A, Ring C, R¹, R^{3A}, R⁴, m, and n are defined in Embodiment 1, the method comprising cross-coupling a compound of Formula (K-1), (K-2), or (K-3), or salt thereof, with a compound of Formula (J-1), or salt thereof:

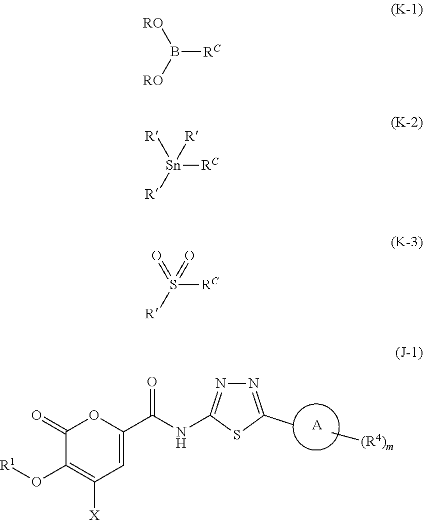

wherein:
   R^{C} is Ring C;
   X is Cl, Br, or I;
   each R is independently H, an optionally substituted C₁₋₆ alkyl, or two R groups are joined to form a 5-6 membered ring; and
   each R′ is independently an optionally substituted C₁₋₆ alkyl;
to provide a compound of Formula (I), or salt thereof.
Embodiment 38. The method of Embodiment 37, wherein the compound of Formula (J-1), or salt thereof, is of Formula (J-2):

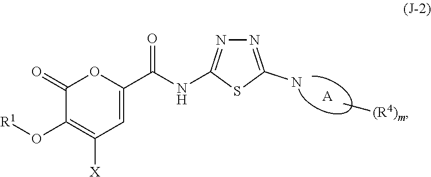

or salt thereof,
and wherein the method provides a compound of Formula (I′):

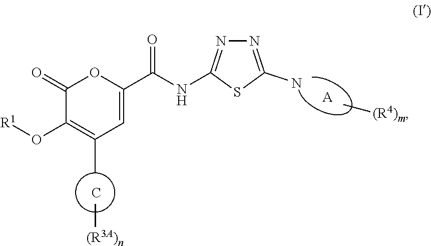

or salt thereof.
Embodiment 39. The method of Embodiment 37, further comprising peptide coupling of a compound of Formula (H-1), or salt thereof, with a compound of Formula (D), or salt thereof:

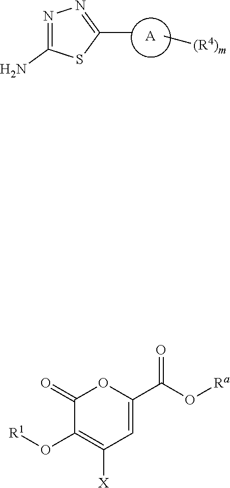

to provide a compound of Formula (J-1), or salt thereof.
Embodiment 40. The method of Embodiment 38, further comprising peptide coupling of a compound of Formula (H-2), or salt thereof, with a compound of Formula (D), or salt thereof:

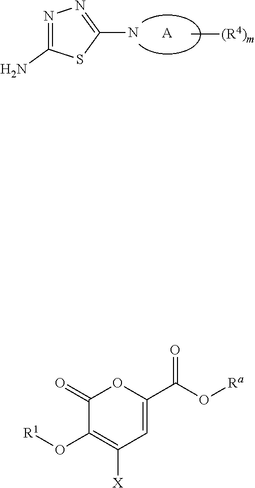

to provide a compound of Formula (J-2), or salt thereof.
Embodiment 41. The method of Embodiment 34 or 39, further comprising reacting a hydrazine carbothioamide of Formula (G), or salt thereof, with a carboxylic acid containing compound of Formula (F-1), or salt thereof, or nitrile containing compound of Formula (F-2), or salt thereof:

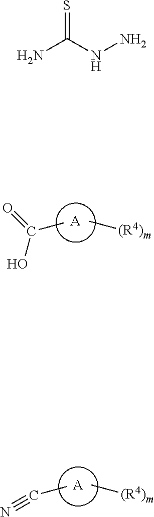

to provide a compound of Formula (H-1), or salt thereof.
Embodiment 42. The method of Embodiment 35 or 40, further comprising coupling a compound Formula (M), or salt thereof, wherein Y is Cl, Br, or I, with an amine of Formula (L), or salt thereof:

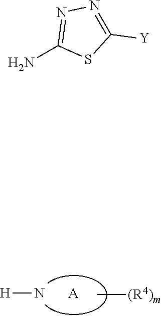

to provide a compound of Formula (H-2), or salt thereof.
Embodiment 43. The method of any one of Embodiments 36, 39, and 40, further comprising:
   (a) Protecting a compound of Formula (A), or salt thereof, to provide an alkyl ester of Formula (B):

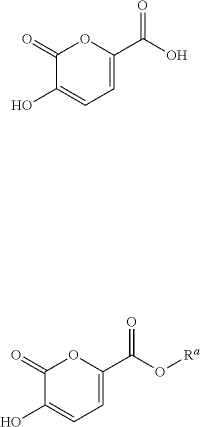

or salt thereof, wherein R^{a} is C₁₋₆ alkyl or C₁₋₆ haloalkyl;
   (b) Halogenating the compound of Formula (B), or salt thereof, to provide a compound of Formula (C):

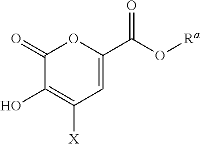

or salt thereof, wherein X is Cl, Br, or I;
   (c) Protecting the hydroxyl group of the compound of Formula (C), or salt thereof, to provide a compound of Formula (D):

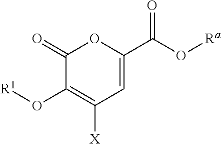

or salt thereof, wherein R¹ is as defined in Embodiment 1; and
   (d) optionally, deprotecting the compound of Formula (D), or salt thereof, to provide a carboxylic acid of Formula (D), wherein R^{a} is hydrogen.

### EXEMPLIFICATION

In order that this disclosure may be more fully understood, the following Examples are set forth. It should be understood that these examples are for illustrative purposes only and are not to be construed as limiting this disclosure in any manner.

### Analytical Methods

Nuclear magnetic resonance (NMR) spectra were recorded at 400 MHz as stated and at 300.3 K unless otherwise stated; the chemical shifts (δ) are reported in parts per million (ppm). Spectra were recorded using a Bruker Avance 400 instrument with 8, 16 or 32 scans. Typical NMR solvents include deuterated dimethylsulfoxide (DMSO-d6) and deuterated methanol (CD₃OD).

Liquid Chromatography-Mass Spectrometry (LCMS) chromatograms and spectra were recorded using a Shimadzu LCMS-2020. Injection volumes were 0.7-8.0 μl and the flow rates were typically 0.8 or 1.2 mL/min. Detection methods were diode array (DAD) or evaporative light scattering (ELSD) as well as positive ion electrospray ionization. MS range was 100-1000 Da. Mobile phases of water and/or acetonitrile (MeCN) may contain a modifier (typically 0.01-0.04%) such as trifluoroacetic acid (TFA), formic acid (FA), ammonia (NH₃·H₂O), or ammonium carbonate (NH₄HCO₃). ESI or ES=electrospray ionization; m/z=mass/charge; RT=retention time (minutes).

Purification/Separation Methods. The synthetic methods describe purification and/or separation chromatographic methods which have been employed in the purification and/or isolation of the exemplified compounds. Rf=retention factor; RT=retention time (minutes); Prep-HPLC=Preparative High-performance liquid chromatography.

### Synthetic Methods

The Asterix (*) next to the Compound Number (#) signifies that arbitrary stereochemistry has been assigned. Future tense (“may be” prepared/synthesized) language signify examples to be conducted.

### Example 1: 4-(2-cyanophenyl)-3-methoxy-N-(5-(5-methyl-1H-pyrazol-1-yl)-1,3,4-thiadiazol-2-yl)-2-oxo-2H-pyran-6-carboxamide (Compound 1)

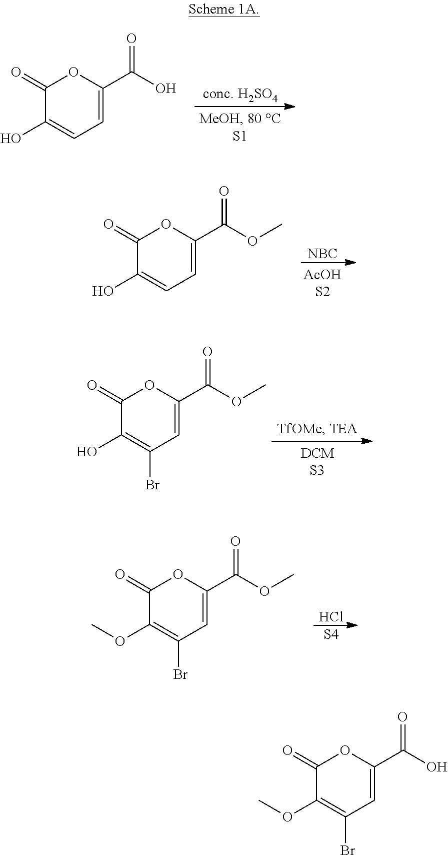

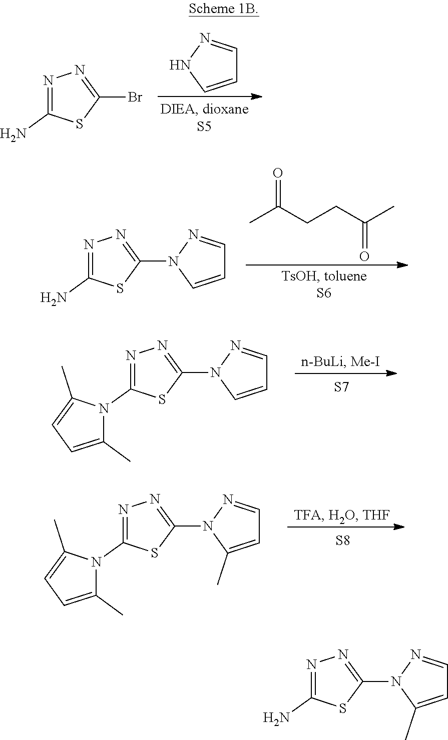

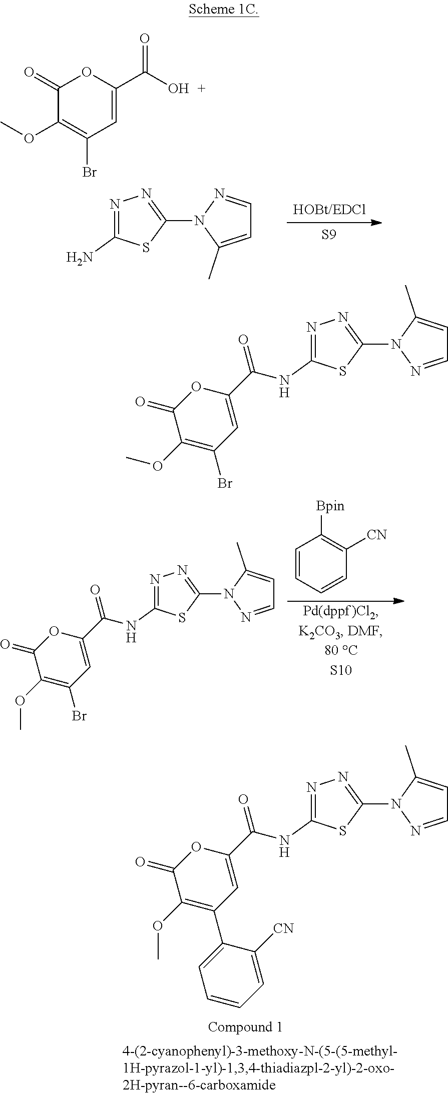

Step 1: Into a solution of 5-hydroxy-6-oxopyran-2-carboxylic acid (180 g, 1153 mmol, 1 equiv) in methanol (MeOH) (2000 mL) was added H₂SO₄ (10 mL, 56 mmol) at room temperature. Then the resulting mixture was stirred overnight at 80° C. The resulting mixture was concentrated under reduced pressure. The residue was then dissolved in ethyl acetate (EtOAc) (1000 mL) and the organic phase was washed water (3×300 mL), and dried over anhydrous Na₂SO₄. After filtration, the filtrate was concentrated under reduced pressure to provide methyl 5-hydroxy-6-oxopyran-2-carboxylate (85 g, 43% yield). LCMS (ES, m/z)=171 [M+1]+.

Step 2: To a stirred solution of methyl 5-hydroxy-6-oxopyran-2-carboxylate (1 g, 5.9 mmol, 1 equiv) in acetic acid (AcOH) (25 mL, 323 mmol) was added N-bromosuccinimide (NBS) (1.25 g, 7.02 mmol, 1.2 equiv) at room temperature. The resulting mixture was stirred for 2 h at 80° C. then diluted with water (70 mL). The resulting mixture was extracted with ethyl acetate (EtOAc) (3×70 mL). The combined organic layers were washed with brine (2×10 mL), and dried over anhydrous Na₂SO₄. After filtration, the filtrate was concentrated under reduced pressure. The resulting residue was purified by silica gel column chromatography, eluting with petroleum ether/ethyl acetate (PE/EtOAc) (3:7), to afford methyl 4-bromo-5-hydroxy-6-oxopyran-2-carboxylate (800 mg, 55% yield). LCMS (ES, m/z)=247 [M−1]−.

Step 3: To a stirred solution of methyl 4-bromo-5-hydroxy-6-oxopyran-2-carboxylate (4 g, 16 mmol, 1 equiv) in dichloromethane (DCM) (50 mL) was added diisopropylethylamine (DIEA) (11 g, 85.1 mmol, 5 equiv) and methyl trifluoromethanesulfonate (TfOMe) (13 g, 79.2 mmol, 5 equiv) dropwise at room temperature. The resulting mixture was stirred for 4 h at room temperature. The mixture was then diluted with water (200 mL) and extracted with DCM (3×200 mL). The combined organic layers were washed with brine (2×30 mL), and dried over anhydrous Na₂SO₄. After filtration, the filtrate was concentrated under reduced pressure. The resulting residue was purified by silica gel column chromatography, eluting with petroleum ether/ethyl acetate (PE/EtOAc) (3:2), to afford methyl 4-bromo-5-methoxy-6-oxopyran-2-carboxylate (3 g, 71% yield). LCMS (ES, m/z)=263 [M+1]+.

Step 4: To methyl 4-bromo-5-methoxy-6-oxopyran-2-carboxylate (10 g, 38.02 mmol, 1 equiv) was added HCl (6M) (200 mL, 65.8 mmol). The mixture was stirred for 4 h at 80° C. then concentrated under reduced pressure to provide 4-bromo-5-methoxy-6-oxopyran-2-carboxylic acid (9.5 g) which was used directly without further purification. LCMS (ES, mz)=249 [M+1]+.

Step 5: A mixture of 5-bromo-1,3,4-thiadiazol-2-amine (200 g, 1110 mmol, 1 equiv), diisopropylethylamine (DIEA) (431 g, 3333 mmol, 3 equiv) and pyrazole (90.76 g, 1333 mmol, 1.2 equiv) in 1,4-dioxane was stirred for 3 h at 80° C. The resulting mixture was concentrated under vacuum and the residue was dissolved in tetrahydrofuran (THF). The mixture was filtered, and the filter cake was washed with THF. The filtrate was concentrated under reduced pressure to afford 5-(pyrazol-1-yl)-1,3,4-thiadiazol-2-amine (100 g, 54% yield), which was used directly in the next step without further purification.

Step 6: A mixture of 5-(pyrazol-1-yl)-1,3,4-thiadiazol-2-amine (100 g, 598 mmol, 1 equiv), tosic acid (TsOH) (20.60 g, 119.6 mmol, 0.2 equiv) and 2,5-hexanedione (102 g, 897 mmol, 1.5 equiv) in toluene was stirred for 2 h at 110° C. The resulting mixture was concentrated under vacuum and the resulting residue was purified by silica gel column chromatography, eluting with petroleum ether/ethyl acetate (PE/EtOAc) (9:1), to afford 2-(2,5-dimethylpyrrol-1-yl)-5-(pyrazol-1-yl)-1,3,4-thiadiazole (90.3 g, 61% yield). LCMS (ES, m/z)=246.1 [M+1]+.

Step 7: A solution of 2-(2,5-dimethylpyrrol-1-yl)-5-(pyrazol-1-yl)-1,3,4-thiadiazole (50 g, 204 mmol, 1 equiv) in tetrahydrofuran (THF) was treated with n-butyl lithium (n-BuLi) (97.8 mL, 245 mmol, 1.2 equiv) for 1 h at −78° C. under N₂ (nitrogen gas) followed by the addition of methyl iodide (CH₃I) (34.7 g, 245 mmol, 1.2 equiv) dropwise at −78° C. The resulting mixture was stirred for 2 h at room temperature under N₂. The reaction was quenched with sat. NH₄Cl (aq.) at 0° C. The resulting mixture was extracted with ethyl acetate (EtOAc). The combined organic layers were washed with brine and dried over anhydrous Na₂SO₄. After filtration, the filtrate was concentrated under reduced pressure. The residue was purified by silica gel column chromatography, eluting with petroleum ether/ethyl acetate (PE/EtOAc) (9:1), to afford 2-(2,5-dimethylpyrrol-1-yl)-5-(5-methylpyrazol-1-yl)-1,3,4-thiadiazole (40 g, 76% yield). LCMS (ES, m/z)=260.0 [M+1]+.

Step 8: To a solution of 2-(2,5-dimethylpyrrol-1-yl)-5-(5-methylpyrazol-1-yl)-1,3,4-thiadiazole (7 g, 27 mmol, 1 equiv) in tetrahydrofuran (THF) (14 mL) and H₂O (28 mL) at room temperature was added trifluoroacetic acid (TFA) (28 mL). The resulting mixture was stirred for 2 h at 50° C. then concentrated under reduced pressure. The residue was purified by C18 reverse phase flash chromatography (acetonitrile (MeCN) in water, 10% to 20% gradient in 10 min; Wave Length: 254 nm) to afford 5-(5-methylpyrazol-1-yl)-1,3,4-thiadiazol-2-amine (3 g, 58% yield). LCMS (ES, m/z)=181.95 [M+1]+.

Step 9: To a stirred solution of 4-bromo-5-methoxy-6-oxopyran-2-carboxylic acid (12 g, 48.2 mmol, 1 equiv) in N,N-dimethylformamide (DMF) (150 mL) was added hydroxybenzotriazole (HOBt) (13.02 g, 96.38 mmol, 2 equiv), 1-ethyl-3-(3-dimethylaminopropyl)carbodiimide (EDCI) (27.81 g, 145.05 mmol, 3 equiv) and 5-(5-methylpyrazol-1-yl)-1,3,4-thiadiazol-2-amine (9 g, 49.7 mmol, 1 equiv) at room temperature. The resulting mixture was stirred for 1 h at room temperature then quenched by the addition of water (70 mL). The precipitated solids were collected by filtration and washed with acetonitrile (5×3 mL) to provide 4-bromo-3-methoxy-N-(5-(5-methyl-1H-pyrazol-1-yl)-1,3,4-thiadiazol-2-yl)-2-oxo-2H-pyran-6-carboxamide (11 g, 55% yield). LCMS (ES, m/z)=412 [M+1]+.

Step 10: To a solution of 4-bromo-3-methoxy-N-(5-(5-methyl-1H-pyrazol-1-yl)-1,3,4-thiadiazol-2-yl)-2-oxo-2H-pyran-6-carboxamide (100 mg, 0.24 mmol, 1 equiv) (“halo-pyrone reagent”) in N,N-dimethylformamide (DMF) (3 mL) was added [1,1′-bis(diphenylphosphino)ferrocene]dichloro palladium(II) (Pd(dppf)Cl₂) (40 mg, 0.055 mmol, 0.2 equiv), 2-(4,4,5,5-tetramethyl-1,3,2-dioxaborolan-2-yl)benzonitrile (80 mg, 0.35 mmol, 1.4 equiv) (“aryl boron reagent”) and K₂CO. (100 mg, 0.72 mmol, 3 equiv). The resulting mixture was stirred for 1 h at 80° C. under nitrogen atmosphere then cooled to rt and diluted with water (20 mL). The resulting mixture was extracted with ethyl acetate (EtOAc) (3×20 mL), and the combined organic layers were washed with brine (4 mL), dried over anhydrous Na₂SO₄ and filtered. After filtration, the filtrate was concentrated under reduced pressure and the resulting crude residue was purified by silica gel column chromatography, eluting with petroleum ether/ethyl acetate (PE/EtOAc) (1:1)), followed by additional purification by Prep-HPLC (Xselect CSH C18 OBD Column 30*150 mm; mobile phase, acetonitrile (MeCN) and water (0.05% TFA) (43% water (0.05% TFA) up to 53% in 10 min); Detector, UV254 nm) to provide 4-(2-cyanophenyl)-3-methoxy-N-(5-(5-methyl-1H-pyrazol-1-yl)-1,3,4-thiadiazol-2-yl)-2-oxo-2H-pyran-6-carboxamide (Compound 1) (13.4 mg, 12.6% yield). LCMS (ESI, m/z)=434.43 [M+H]⁺. ¹H NMR (400 MHz, DMSO-d₆) δ 8.04 (d, J=7.6 Hz, 1H), 7.89 (dd, J=8.4, 7.2 Hz, 1H), 7.79 (d, J=1.6 Hz, 1H), 7.75-7.63 (m, 2H), 7.56 (s, 1H), 6.45 (d, J=1.2 Hz, 1H), 3.95 (s, 3H), 2.68 (s, 3H).

### Example 2: 4-(2-(dimethylphosphoryl)phenyl)-3-methoxy-N-(5-(5-methyl-1H-pyrazol-1-yl)-1,3,4-thiadiazol-2-yl)-2-oxo-2H-pyran-6-carboxamide (Compound 3)

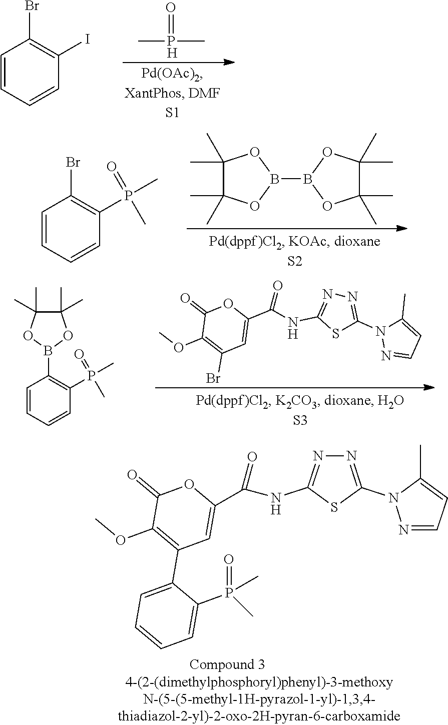

Step 1: To a stirred solution of 1-bromo-2-iodobenzene (500 mg, 1.77 mmol, 1 equiv) in N,N-dimethylformamide (DMF) (5 mL) was added palladium (11) acetate (Pd(OAc)₂) (40 mg, 0.18 mmol, 0.1 equiv), 4,5-bis(diphenylphosphino)-9,9-dimethylxanthene (XantPhos) (205 mg, 0.35 mmol, 0.2 equiv), K₃PO₄ (566 mg, 2.67 mmol, 1.5 equiv) and (methylphosphonoyl)methane (166 mg, 2.13 mmol, 1.2 equiv) at room temperature under nitrogen atmosphere. The resulting mixture was stirred for 3 h at 90° C. under nitrogen atmosphere then cooled to rt and concentrated under reduced pressure. The crude residue was then purified by C18 reverse phase flash chromatography (mobile phase, MeCN in water, 30% to 70% gradient in 10 min; detector, UV 254 nm) to provide 1-bromo-2-(dimethylphosphoryl)benzene (330 mg, 80% yield). LCMS (ES, m/z)=233.0 [M+H]+.

Step 2: To a stirred solution of 1-bromo-2-(dimethylphosphoryl)benzene (300 mg, 1.29 mmol, 1 equiv) in dioxane (3 mL) was added [1,1′-bis(diphenylphosphino)ferrocene]dichloro palladium(II) (Pd(dppf)Cl₂) (180 mg, 0.25 mmol, 0.2 equiv), potassium acetate (AcOK) (390 mg, 3.97 mmol, 3 equiv) and bis(pinacolato)diboron (3.30 g, 13.0 mmol, 10 equiv) at room temperature under nitrogen atmosphere. The resulting mixture was stirred for 4 h at 80° C. under nitrogen atmosphere then cooled to rt and directly purified by C18 reverse phase flash chromatography (mobile phase, MeCN in water, 10% to 50% gradient in 10 min; detector, UV 254 nm) to provide 2-[2-(dimethylphosphoryl)phenyl]-4,4,5,5-tetramethyl-1,3,2-dioxaborolane (160 mg, 44% yield). LCMS (ESI, m/z)=280.1 [M+1]+.

Step 3: 4-(2-(dimethylphosphoryl)phenyl)-3-methoxy-N-(5-(5-methyl-1H-pyrazol-1-yl)-1,3,4-thiadiazol-2-yl)-2-oxo-2H-pyran-6-carboxamide (Compound 3) was prepared according to Example 1, Step 10 using 2-[2-(dimethylphosphoryl)phenyl]-4,4,5,5-tetramethyl-1,3,2-dioxaborolane as the “aryl boron reagent” and 4-bromno-3-methoxy-N-(5-(5-methyl-1H-pyrazol-1-yl)-1,3,4-thiadiazol-2-yl)-2-oxo-2H-pyran-6-carboxamide (product of step 10, Example 1) as the “halo-pyrone reagent” and dioxane/H₂O (10:1) as solvent in place of N,N-dimethylformamide (DMF). LCMS (ES, m/z)=486.00 [M+H]⁺. ¹H NMR (400 MHz, DMSO-d6) δ 13.44 (br, 1H), 7.90-7.80 (m, 1H), 7.77 (d, J=1.6 Hz, 1H), 7.72-7.53 (m, 2H), 7.44-7.29 (m, 2H), 6.43 (d, J=1.2 Hz, 1H), 3.79 (s, 3H), 2.67 (s, 3H), 1.72-1.62 (m, 6H).

### Example 3: 3-(2-methoxyethoxy)-4-(3-methoxypyridin-2-yl)-N-(5-(5-methyl-1H-pyrazol-1-yl)-1,3,4-thiadiazol-2-yl)-2-oxo-2H-pyran-6-carboxamide (Compound 4)

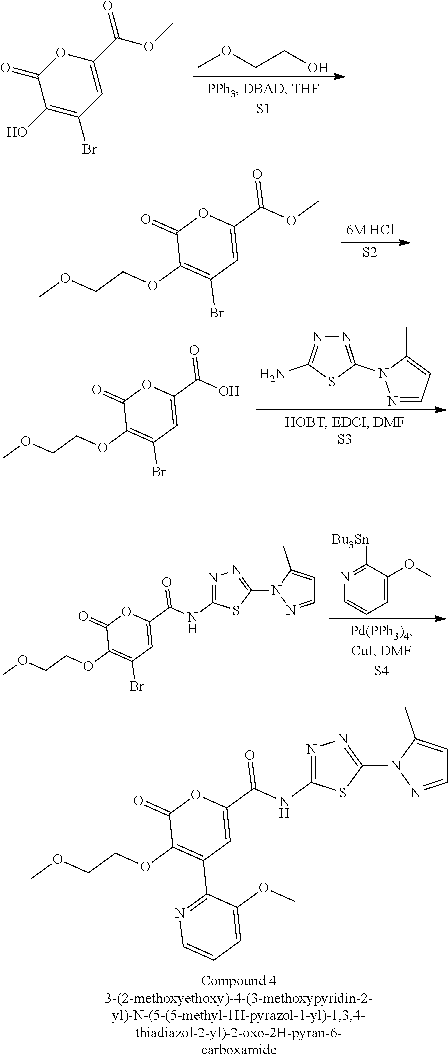

Step 1: To a stirred solution of methyl 4-bromo-5-hydroxy-6-oxopyran-2-carboxylate (product of Step 2, Example 1) (5 g, 20 mmol, 1 equiv) and 2-methoxyethanol (2 g, 26 mmol, 1.3 equiv) in tetrahydrofuran (THF) was added triphenyl phosphine (PPh₃) (8 g, 30 mmol, 1.5 equiv) in portions at room temperature. The resulting mixture was stirred for 10 min at 0° C. To this was then added di-tert-butyl azodicarboxylate (DBAD) (7 g, 30 mmol, 1.5 equiv) dropwise at room temperature. The resulting mixture was stirred overnight at room temperature. The mixture was then diluted with ethyl acetate (EtOAc) (600 mL), washed with water (3×200 mL), and dried over anhydrous Na₂SO₄. After filtration, the filtrate was concentrated under reduced pressure and the residue was purified by silica gel column chromatography, eluting with petroleum ether/ethyl acetate (PE/EtOAc) (1:3), to afford methyl 4-bromo-3-(2-methoxyethoxy)-2-oxo-2H-pyran-6-carboxylate (3.7 g, 60% yield). LCMS (ESI, m/z)=307,309 [M+1]+.

Step 2: A solution of methyl 4-bromo-3-(2-methoxyethoxy)-2-oxo-2H-pyran-6-carboxylate (1700 mg, 5.54 mmol, 1 equiv) in HCl (6M) (30 mL) was stirred for 3 h at 80° C. The resulting mixture was then concentrated under reduced pressure and diluted with ethyl acetate (EtOAc) (200 mL), washed with brine (3×10 mL), and dried over anhydrous Na₂SO₄. After filtration, the filtrate was concentrated under reduced pressure to provide 4-bromo-3-(2-methoxyethoxy)-2-oxo-2H-pyran-6-carboxylic acid, which was used directly in the next step without further purification. LCMS (ESI, mz)=293.0 [M+1]+.

Step 3: To a stirred solution of 4-bromo-3-(2-methoxyethoxy)-2-oxo-2H-pyran-6-carboxylic acid (1200 mg, 4.09 mmol, 1 equiv) and 5-(5-methylpyrazol-1-yl)-1,3,4-thiadiazol-2-amine (product of Step 8, Example 1) (820 mg, 4.52 mmol, 1 equiv) in N,N-dimethylformamide (DMF) (21 mL) was added hydroxybenzotriazole (HOBT) (1110 mg, 8.22 mmol, 2 equiv) and 1-ethyl-3-(3-dimethylaminopropyl)carbodiimide (EDCI) (2355 mg, 12.3 mmol, 3 equiv) at room temperature. The resulting mixture was stirred for 1 h at room temperature before being diluted with water (40 mL). The precipitated solids were collected by filtration to afford 4-bromo-5-(2-methoxyethoxy)-N-[5-(5-methylpyrazol-1-yl)-1,3,4-thiadiazol-2-yl]-6-oxopyran-2-carboxamide (1.3 g, 70% yield). LCMS (ESI, m/z)=458.05 [M+1]+.

Step 4: To a stirred solution of 4-bromo-5-(2-methoxyethoxy)-N-[5-(5-methylpyrazol-1-yl)-1,3,4-thiadiazol-2-yl]-6-oxopyran-2-carboxamide (“halo-pyrone reagent”) (80 mg, 0.18 mmol, 1 equiv) in N,N-dimethylformamide (DMF) (2 mL) was added 3-methoxy-2-(tributylstannyl)pyridine (“aryl tin reagent”) (110 mg, 0.28 mmol, 1.58 equiv), tetrakis (triphenylphosphine)palladium (0) (Pd(PPh₃)₄) (50 mg, 0.043 mmol, 0.25 equiv) and CuI (10 mg, 0.053 mmol, 0.30 equiv) at room temperature. The resulting mixture was stirred for 2 h at 110° C. under nitrogen atmosphere then cooled to it and diluted with water (20 mL). The resulting mixture was extracted with ethyl acetate (EtOAc) (3×30 mL) and then the combined organic layers were washed with brine (2×10 mL), and dried over anhydrous Na₂SO₄. After filtration, the filtrate was concentrated under reduced pressure and the resulting residue was purified by C18 reverse phase flash chromatography (mobile phase, MeCN in water (0.05% TFA), 10% to 50% gradient in 10 min; detector. UV 254 nm) to provide a residue which was further purified by trituration with acetonitrile (MeCN) (2 mL). The precipitated solids were collected by filtration and washed with MeCN (2×2 mL) to provide 3-(2-methoxyethoxy)-4-(3-methoxypyridin-2-yl)-N-(5-(5-methyl-1H-pyrazol-1-yl)-1,3,4-thiadiazol-2-yl)-2-oxo-2H-pyran-6-carboxamide (Compound 4) (28 mg, 32% yield). LCMS (ES, m/z)=484.95 [M+H]+. ¹H NMR (400 MHz, DMSO-d6) δ 13.42 (br, 1H), 8.31 (d, J=4.8 Hz, 1H), 7.78 (s, 1H), 7.65 (dd. J=8.4, 1.2 Hz, 1H), 7.56-7.48 (in, 2H), 6.44 (d, J=1.6 Hz 1H), 4.30-4.23 (m, 2H), 3.86 (s, 3H), 3.46-3.40 (m, 2H), 3.10 (s, 3H), 2.67 (s, 3H).

### Example 4: N-(5-(3-chloro-1-methyl-1H-pyrrol-2-yl)-1,3,4-thiadiazol-2-yl)-4-(2,6-dimethoxyphenyl)-3-(2-methoxyethoxy)-2-oxo-2H-pyran-6-carboxamide (Compound 5)

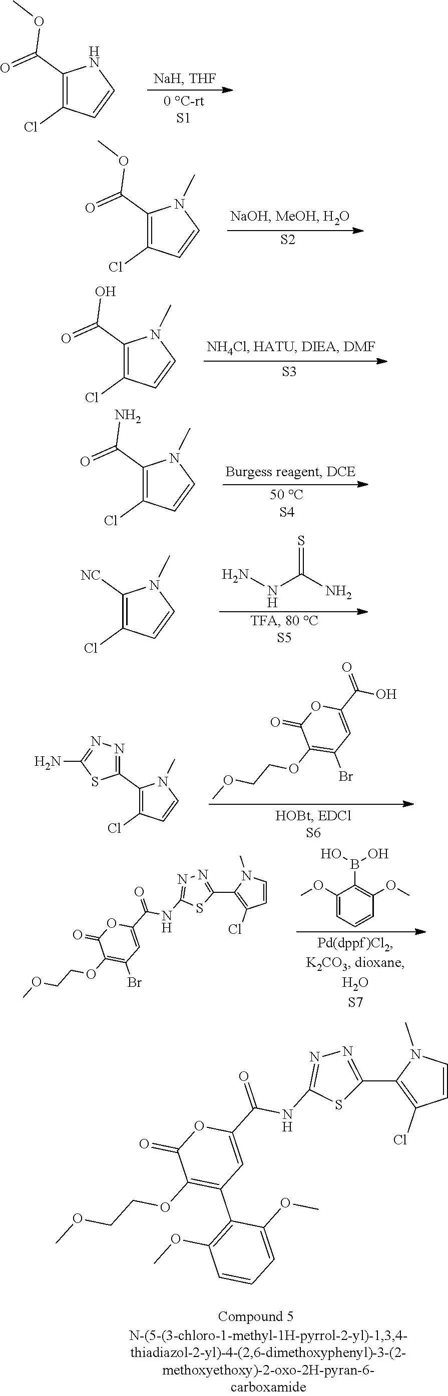

Step 1: Into a 250 mL round-bottom flask were added methyl 3-chloro-1H-pyrrole-2-carboxylate (2.40 g, 15.0 mmol, 1 equiv) and tetrahydrofuran (THF) (20 mL, 247 mmol, 16.4 equiv) at room temperature. To the above mixture was added NaH (1.44 g, 60.0 mmol, 4 equiv) in portions at 0° C. The resulting mixture was stirred for an additional 1 h at 0° C. To the above mixture was then added methyl iodide (MeI) (6.48 g, 45.6 mmol, 3 equiv) dropwise at 0° C. The resulting mixture was then stirred overnight at room temperature. The reaction was then quenched by the addition of HCl (1N) (40 mL) at 0° C. The resulting mixture was extracted with ethyl acetate (EtOAc) (3×40 mL). The combined organic layers were washed with water (2×20 mL), dried over anhydrous Na₂SO₄. After filtration, the filtrate was concentrated under reduced pressure. The residue was purified by silica gel column chromatography, eluting with petroleum ether/ethyl acetate (PE/EtOAc) (1:1), to afford methyl 3-chloro-1-methyl-1H-pyrrole-2-carboxylate (1.7 g, 71% yield). LCMS (ESI, m/z)=173.85 [M+1]+.

Step 2: Into a 40 mL vial was added methyl 3-chloro-1-methyl-1H-pyrrole-2-carboxylate (750 mg, 4.32 mmol, 1 equiv) and methanol (MeOH) (4.0 mL) at room temperature. To the above mixture was added NaOH (330 mg, 8.25 mmol, 2 equiv) in H₂O (4.0 mL) at room temperature. The resulting mixture was stirred for an additional 2 h at 50° C. The mixture was then acidified to pH 6 with HCl (3 M). The resulting mixture was extracted with ethyl acetate (EtOAc) (3×30 mL). The combined organic layers were washed with water (2×10 mL), and dried over anhydrous Na₂SO₄. After filtration, the filtrate was concentrated under reduced pressure to afford 3-chloro-1-methylpyrrole-2-carboxylic acid (580 mg, 80% yield). LCMS (ESI, m/z)=160.0 [M+1]+.

Step 3: Into a 40 mL vial was added 3-chloro-1-methylpyrrole-2-carboxylic acid (2.2 g, 13.8 mmol, 1 equiv), N,N-dimethylformamide (DMF) (20 mL), 1-[bis(dimethylamino)methylene]-1H-1,2,3-triazolo[4,5-b]pyridinium 3-oxid hexafluorophosphate (HATU) (15.7 g, 41.4 mmol, 3 equiv), diisopropylethylamine (DIEA) (5.42 g, 41.9 mmol, 3 equiv) and NH₄Cl (2.97 g, 55.6 mmol, 4 equiv) at room temperature. The resulting mixture was stirred for 3 h at 80° C. The reaction was then quenched with water at room temperature and the aqueous layer was extracted with ethyl acetate (EtOAc) (3×30 mL). The combined organic layers were washed with brine (3×30 mL) then concentrated under reduced pressure. The resulting residue was purified by C18 reverse phase flash chromatography (acetonitrile (MeCN) in water, 20% to 50% gradient in 10 min; detector, UV 254 nm) to afford 3-chloro-1-methylpyrrole-2-carboxamide (1.47 g, 60% yield). LCMS (ESI, m/z)=159.05 [M+1]+.

Step 4: Into a 40 mL vial was added 3-chloro-1-methylpyrrole-2-carboxamide (1.40 g, 8.83 mmol, 1 equiv), dichloroethane (DCE) (20 mL) and methyl N-(triethylammoniumsulfonyl)carbamate (Burgess reagent) (6.29 g, 26.4 mmol, 3 equiv) at room temperature. The resulting mixture was stirred for 2 h at 50° C. The reaction was then quenched with water at room temperature and extracted with dichloromethane (DCM) (3×20 mL). The combined organic layers were washed with water (3×20 mL), and dried over anhydrous Na₂SO₄. After filtration, the filtrate was concentrated under reduced pressure and the residue purified by silica gel column chromatography, eluting with petroleum ether/ethyl acetate (PE/EtOAc) (70:30), to afford 3-chloro-1-methylpyrrole-2-carbonitrile (940 mg, 68% yield).

Step 5: A mixture of 3-chloro-1-methylpyrrole-2-carbonitrile (100 mg, 0.71 mmol, 1 equiv) and thiosemicarbazide (200 mg, 2.19 mmol, 3.1 equiv) in trifluoroacetic acid (TFA) (5 mL) was stirred for 16 h at 80° C. The resulting mixture was concentrated under reduced pressure and the residue was purified by C18 reverse phase flash chromatography (acetonitrile (MeCN) in water, 30% to 40% gradient in 10 min; detector, UV 254 nm) to afford 5-(3-chloro-1-methylpyrrol-2-yl)-1,3,4-thiadiazol-2-amine (45 mg, 26% yield). LCMS (ESI, m/z)=215.0 [M+1]+.

Step 6: To a stirred solution of 4-bromo-3-(2-methoxyethoxy)-2-oxo-2H-pyran-6-carboxylic acid (product of Step 2, Example 3) (300 mg, 1.02 mmol, 1 equiv) and 5-(3-chloro-1-methylpyrrol-2-yl)-1,3,4-thiadiazol-2-amine (270 mg, 1.26 mmol, 1.23 equiv) in N,N-dimethylformamide (DMF) (6 mL) was added hydroxybenzotriazole (HOBT) (270 mg, 2 mmol, 1.95 equiv) and 1-ethyl-3-(3-dimethylaminopropyl)carbodiimide (EDCI) (600 mg, 3.13 mmol, 3.06 equiv) in portions at room temperature. The resulting mixture was stirred for 1 h at room temperature then water (30 mL) was added. The resulting solids were collected by filtration and washed with water (2×2 mL) to provide 4-bromo-N-[5-(3-chloro-1-methylpyrrol-2-yl)-1,3,4-thiadiazol-2-yl]-5-(2-methoxyethoxy)-6-oxopyran-2-carboxamide (390 mg, 78% yield). LCMS (ES, m/z)=491.10 [M+1]+.

Step 7: Into a 20 mL vial was added 4-bromo-N-[5-(3-chloro-1-methylpyrrol-2-yl)-1,3,4-thiadiazol-2-yl]-5-(2-methoxyethoxy)-6-oxopyran-2-carboxamide (“halo-pyrone reagent”) (80 mg, 0.16 mmol, 1 equiv), 2,6-dimethoxyphenylboronic acid (“aryl boron reagent”) (36 mg, 0.2 mmol, 1.2 equiv), [1,1′-bis(diphenylphosphino)ferrocene]dichloro palladium(II) (Pd(dppf)Cl₂)—CH₂Cl₂ complex (26.6 mg, 0.033 mmol, 0.2 equiv), K₃PO₄ (104 mg, 0.49 mmol, 3 equiv), dioxane (1.5 mL), and H₂O (0.3 mL). The resulting mixture was stirred for 30 min at 80° C. under nitrogen atmosphere. The mixture was then cooled to rt and extracted with ethyl acetate (EtOAc) (3×20 mL), and dried over anhydrous Na₂SO₄. After filtration, the filtrate was concentrated under reduced pressure and the crude material was purified by Prep-HPLC (XBridge Shield RP18 OBD Column, 30*150 mm; mobile phase, water (10 mmol/L NH₄HCO₃) and acetonitrile (MeCN) (33% MeCN up to 43% in 10 min); Detector, UV) to provide N-(5-(3-chloro-1-methyl-1H-pyrrol-2-yl)-1,3,4-thiadiazol-2-yl)-4-(2,6-dimethoxyphenyl)-3-(2-methoxyethoxy)-2-oxo-2H-pyran-6-carboxamide (Compound 5) (8.8 mg, 9.5% yield). LCMS (ES, m/z): 547.10 [M+H]⁺. ¹H NMR (400 MHz, DMSO-d6) δ 13.27 (s, 1H), 7.43 (t, J=8.4 Hz, 1H), 7.27 (s, 1H), 7.17 (d, J=2.9 Hz, 1H), 6.79 (d, J=8.5 Hz, 2H), 6.34 (d, J=2.9 Hz, 1H), 4.21-4.15 (m, 2H), 3.95 (s, 3H), 3.78 (s, 6H), 3.43-3.36 (m, 2H), 3.10 (s, 3H).

### Example 5: N-(5-(5-amino-1H-pyrazol-1-yl)-1,3,4-thiadiazol-2-yl)-4-(2,6-dimethoxyphenyl)-3-(2-methoxyethoxy)-2-oxo-2H-pyran-6-carboxamide (Compound 6) and N-(5-(5-acetamido-1H-pyrazol-1-yl)-1,3,4-thiadiazol-2-yl)-4-(2,6-dimethoxyphenyl)-3-(2-methoxyethoxy)-2-oxo-2H-pyran-6-carboxamide (Compound 6-Ac)

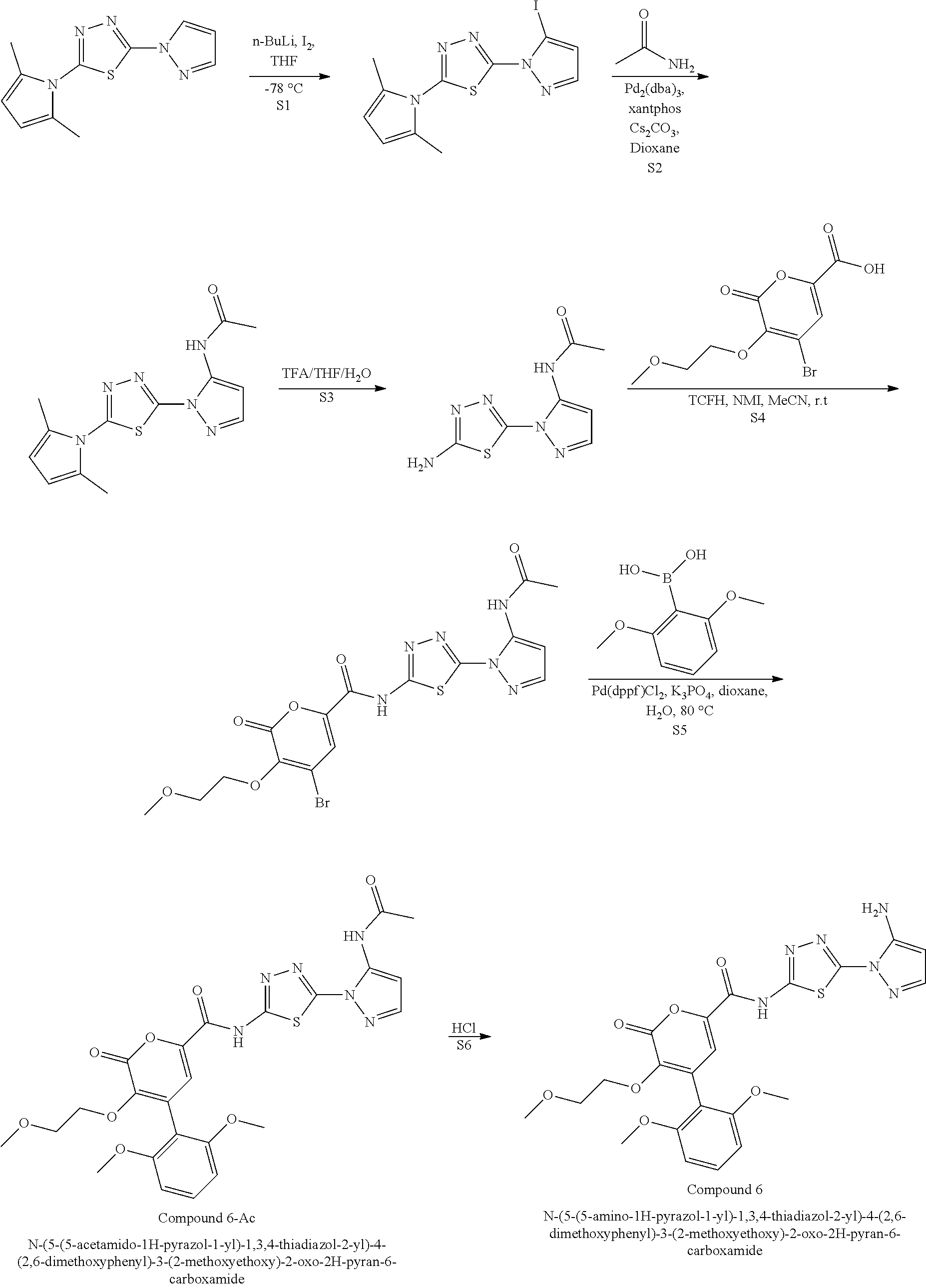

Step 1: To a stirred solution of 2-(2,5-dimethylpyrrol-1-yl)-5-(pyrazol-1-yl)-1,3,4-thiadiazole (6 g, 24.46 mmol, 1 equiv) in tetrahydrofuran (THF) (70 mL) was added n-butyl lithium (n-BuLi) (11.74 mL, 29.35 mmol, 1.2 equiv, 2.5 M) in portions at −78° C. under N₂ (nitrogen gas). The resulting mixture was stirred for 1 h at −78° C. under N₂. And then to the above mixture was added I₂ (18.62 g, 73.38 mmol, 3 equiv) in THF (20 mL) in portions over 30 min at −78° C. under N₂. The resulting mixture was stirred for additional 40 min at −78° C. and then the resulting mixture was stirred for 2 h at room temperature under N₂. The reaction was then quenched with sat. NH₄Cl (aq.) at room temperature. The mixture was extracted with ethyl acetate (EtOAc) (3×100 mL), washed with sat. Na₂S₂O₃ (2×100 mL), then with brine (2×100 mL), and dried over anhydrous Na₂SO₄. After filtration, the filtrate was concentrated under reduced pressure. The residue was purified by silica gel column chromatography, eluting with petroleum ether/ethyl acetate (PE/EtOAc) (15:1), to afford 2-(2,5-dimethylpyrrol-1-yl)-5-(5-iodopyrazol-1-yl)-1,3,4-thiadiazole (6.8 g, 75% yield). LCMS (ES, m/z)=372 [M+1]+.

Step 2: A mixture of 2-(2,5-dimethylpyrrol-1-yl)-5-(5-iodopyrazol-1-yl)-1,3,4-thiadiazole (2 g, 5.39 mmol, 1 equiv), tris(dibenzylidenaceton)dipalladium(0) dibenzylidenacetone (Pd₂(dba)₃) (494 mg, 0.539 mmol, 0.1 equiv), (9,9-dimethyl-9H-xanthene-4,5-diyl)bis(diphenylphosphane) (XantPhos) (624 mg, 1.078 mmol, 0.2 equiv), Cs₂CO₃ (3.53 g, 10.8 mmol, 2 equiv) and acetamide (962 mg, 16.3 mmol, 3 equiv) in dioxane (25 mL) was stirred for 1 h at 120° C. under N₂ then concentrated under vacuum. The resulting residue was purified by silica gel column chromatography, eluting with petroleum ether/ethyl acetate (PE/EtOAc) (8:1), to afford N-{2-[5-(2,5-dimethylpyrrol-1-yl)-1,3,4-thiadiazol-2-yl]pyrazol-3-yl}acetamide (1.1 g, 67% yield). LCMS (ES, m/z)=303 [M+1]+.

Step 3: To a stirred solution of N-{2-[5-(2,5-dimethylpyrrol-1-yl)-1,3,4-thiadiazol-2-yl]pyrazol-3-yl}acetamide (1.10 g, 3.64 mmol, 1 equiv) in tetrahydrofuran (THF) (4 mL) and H₂O (4 mL) was slowly added trifluoroacetic acid (TFA) (8 mL) dropwise at 0° C. The resulting mixture was stirred overnight at room temperature then volatiles removed under a stream of N₂. The residue was then diluted with dichloromethane (DCM) (3 mL). The precipitated solids were collected by filtration and washed with DCM (2×1 mL) to afford N-(1-(5-amino-1,3,4-thiadiazol-2-yl)-1H-pyrazol-5-yl)acetamide (400 mg, 49% yield). LCMS (ES, m/z)=225 [M+1]+.

Step 4: To a stirred solution of 4-bromo-3-(2-methoxyethoxy)-2-oxo-2H-pyran-6-carboxylic acid (product of Step 2, Example 3) (300 mg, 1.02 mmol, 1 equiv) and N-(1-(5-amino-1,3,4-thiadiazol-2-yl)-1H-pyrazol-5-yl)acetamide (253 mg, 1.13 mmol, 1.1 equiv) in acetonitrile (MeCN) (6 mL) was added chloro-N,N,N′,N′-tetramethylformamidinium hexafluorophosphate (TCFH) (575 mg, 2.05 mmol, 2 equiv) and N-methylimidazole (NMI) (842 mg, 10.2 mmol, 10 equiv) in portions at room temperature. The resulting mixture was stirred for 1 h at room temperature followed by addition of water (4 mL). The resulting mixture was filtered, the filtrate was concentrated under reduced pressure, and the residue was purified by trituration with MeCN (1 mL) and H₂O (1 mL) to provide 4-bromo-N-[5-(5-acetamidopyrazol-1-yl)-1,3,4-thiadiazol-2-yl]-5-(2-methoxyethoxy)-6-oxopyran-2-carboxamide (160 mg, 31% yield). LCMS (ES, m/z)=499 [M+H]+.

Step 5: To a stirred solution of 4-bromo-N-[5-(5-acetamidopyrazol-1-yl)-1,3,4-thiadiazol-2-yl]-5-(2-methoxyethoxy)-6-oxopyran-2-carboxamide (“halo-pyrone reagent”) (300 mg, 0.60 mmol, 1 equiv) and 2,6-dimethoxyphenylboronic acid (“aryl boron reagent”) (164 mg, 0.90 mmol, 1.5 equiv) in dioxane (3 mL) and H₂O (0.6 mL) was added [1,1′-bis(diphenylphosphino)ferrocene]dichloro palladium(II) (Pd(dppf)Cl₂) (87 mg, 0.12 mmol, 0.2 equiv) and K₃PO₄ (382 mg, 1.80 mmol, 3 equiv) in portions at room temperature. The resulting mixture was stirred for 1 h at 80° C. under nitrogen atmosphere then cooled to rt and purified directly by C18 reverse phase flash chromatography (mobile phase, MeCN in water, 10% to 50% gradient in 10 min; detector, UV 254 nm) to provide N-(5-(5-acetamido-1H-pyrazol-1-yl)-1,3,4-thiadiazol-2-yl)-4-(2,6-dimethoxyphenyl)-3-(2-methoxyethoxy)-2-oxo-2H-pyran-6-carboxamide (Compound 6-Ac) (200 mg, 60% yield). LCMS (ES, m/z)=557.0 [M+H]+.

Step 6: To N-(5-(5-acetamido-1H-pyrazol-1-yl)-1,3,4-thiadiazol-2-yl)-4-(2,6-dimethoxyphenyl)-3-(2-methoxyethoxy)-2-oxo-2H-pyran-6-carboxamide (Compound 6-Ac) (200 mg, 0.36 mmol, 1 equiv) was added cone. HCl (5 mL) at room temperature. The resulting mixture was stirred for 2 h at room temperature. Water was then added and the resulting solids collected by filtration and washed with water (3×20 mL). The collected solids were further purified by C18 reverse phase flash chromatography (mobile phase, MeCN in water (10 mmol/L NH₄HCO₃), 10% to 50% gradient in 10 min; detector, UV 254 nm) to provide N-[5-(5-aminopyrazol-1-yl)-1,3,4-thiadiazol-2-yl]-4-(2,6-dimethoxyphenyl)-5-(2-methoxyethoxy)-6-oxopyran-2-carboxamide (Compound 6) (25 mg, 13% yield). LCMS (ES, m/z): 515.10 [M+1]+; 1H NMR (400 MHz, DMSO-d6) δ 7.55-7.35 (m, 2H), 7.27 (s, 1H), 6.79 (d, J=8.4 Hz, 2H), 6.65 (s, 2H), 5.44 (d, J=1.9 Hz, 1H), 4.18 (t, J=4.6 Hz, 2H), 3.77 (s, 6H), 3.39 (s, 2H), 3.10 (s, 3H).

### Example 6: 4-(3-cyanopyridin-2-yl)-3-(2-methoxyethoxy)-N-(5-(5-methyl-1H-pyrazol-1-yl)-1,3,4-thiadiazol-2-yl)-2-oxo-2H-pyran-6-carboxamide (Compound 7)

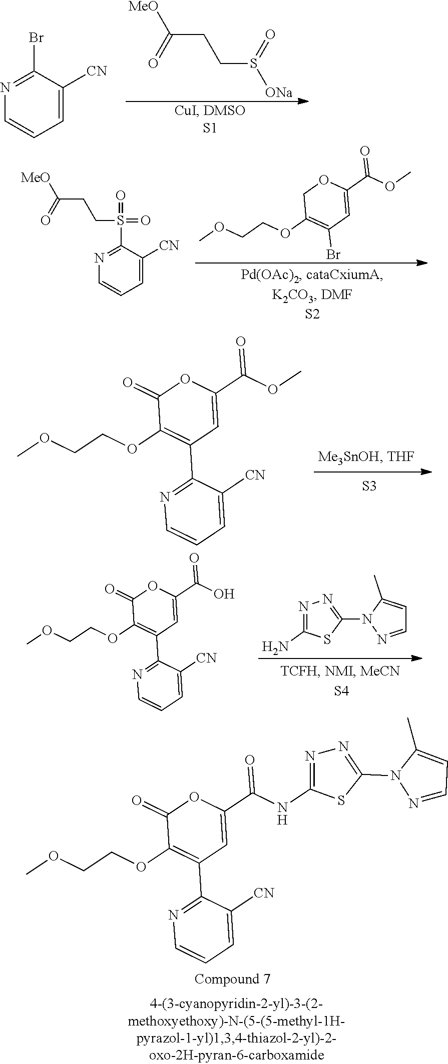

Step 1: To a stirred solution of 2-bromopyridine-3-carbonitrile (3 g, 16 mmol, 1 equiv) and methyl 3-(sodiooxysulfinyl)propanoate (8.56 g, 49.2 mmol, 3 equiv) in DMSO (20 mL) was added CuI (9.37 g, 49.2 mmol, 3 equiv) at room temperature. The resulting mixture was stirred for 1 h at 110° C. under nitrogen atmosphere. The mixture was then cooled to rt and filtered. The filtrate was diluted with ethyl acetate (EtOAc) (50 mL) and water (20 mL) and then extracted with EtOAc (3×100 mL). The organic layers were combined and washed with water (3×10 mL) then brine (20 mL), dried over Na₂SO₄ and concentrated under vacuum. The resulting residue was purified by silica gel column chromatography (eluting with petroleum ether/ethyl acetate (PE/EtOAc) (1:1)) to afford methyl 3-(3-cyanopyridin-2-ylsulfonyl)propanoate (2.8 g, 67% yield). LCMS (ES, m/z)=407.10 [M+H]+.

Step 2: To a stirred solution of methyl 4-bromo-5-(2-methoxyethoxy)-6-oxopyran-2-carboxylate (product of Step 1, Example 3) (2.7 g, 8.8 mmol, 1 equiv) in N,N-dimethylformamide (DMF) (30 mL) was added palladium (II) acetate (Pd(OAc)₂) (0.5 g, 2.2 mmol, 0.2 equiv), butylbis[(3R,5S,7s)-adamantan-1-yl]phosphane (cataCXiumA) (0.9 g, 2.5 mmol, 0.3 equiv), K₂CO₃ (3.6 g, 26 mmol, 3 equiv) and methyl 3-(3-cyanopyridin-2-ylsulfonyl)propanoate (2.70 g, 10.6 mmol, 1.2 equiv) at room temperature. The resulting mixture was stirred for 2 h at 100° C. under nitrogen atmosphere. The mixture was then cooled to rt and diluted with ethyl acetate (EtOAc) (100 mL) and water (30 mL). The mixture was then extracted with EtOAc (3×100 mL) and the organic layers combined. The combined organic extracts were washed with water (3×20 mL) then brine (50 mL), and concentrated under reduced pressure. The resulting residue was then purified by C18 reverse phase flash chromatography (mobile phase, MeCN in water (0.1% TFA), 0% to 100% gradient in 10 min; detector, UV 254 nm) to provide methyl 4-(3-cyanopyridin-2-yl)-5-(2-methoxyethoxy)-6-oxopyran-2-carboxylate (480 mg, 16% yield). LCMS (ES, m/z)=331 [M+H]+.

Step 3: To a stirred solution of methyl 4-(3-cyanopyridin-2-yl)-5-(2-methoxyethoxy)-6-oxopyran-2-carboxylate (100 mg, 0.30 mmol, 1 equiv) in tetrahydrofuran (THF) (5 mL) was added trimethyltin hydroxide (170 mg, 0.94 mmol, 3 equiv) at room temperature. The resulting mixture was stirred for 1 h at room temperature then concentrated under reduced pressure to provide crude 4-(3-cyanopyridin-2-yl)-5-(2-methoxyethoxy)-6-oxopyran-2-carboxylic acid, which was used directly in the next step without further purification. LCMS (ES, m/z)=317 [M+H]+.

Step 4: To a stirred solution of 5-(5-methylpyrazol-1-yl)-1,3,4-thiadiazol-2-amine (product of Step 8, Example 1; “ADT amine reagent”) (65 mg, 0.36 mmol, 1.19 equiv) in acetonitrile (MeCN) (2 mL) was added chloro-N,N,N′,N′-tetramethylformamidinium hexafluorophosphate (TCFH) (130 mg, 0.46 mmol, 1.54 equiv), N-methylimidazole (NMI) (50 mg, 0.61 mmol, 2.03 equiv) and 4-(3-cyanopyridin-2-yl)-5-(2-methoxyethoxy)-6-oxopyran-2-carboxylic acid (“aryl-pyrone reagent”) (95 mg, 0.30 mmol, 1 equiv) at room temperature. The resulting mixture was stirred for 2 h at room temperature then concentrated under reduced pressure. The resulting residue was purified by C18 reverse phase flash chromatography (mobile phase, MeCN in water, 10% to 50% gradient in 10 min; detector, UV 254 nm) followed by additional purification by Prep-HPLC (XBridge Prep OBD C18 Column, 30*150 mm; Mobile Phase A: Water (10 mmol/L NH₄HCO₃), Mobile Phase B: acetonitrile (MeCN); Flow rate: 60 mL/min; Gradient: 17% B to 27% B in 8 min, 27% B; Wave Length: 220/254 nm) to afford 4-(3-cyanopyridin-2-yl)-3-(2-methoxyethoxy)-N-(5-(5-methyl-1H-pyrazol-1-yl)-1,3,4-thiadiazol-2-yl)-2-oxo-2H-pyran-6-carboxamide (Compound 7) (1.1 mg, 0.7% yield). LCMS (ES, m/z)=480.05 [M+H]+; ¹H NMR (400 MHz, DMSO-d6) δ 9.02 (d, J=5.2 Hz, 1H), 8.51 (d, J=8.0 Hz, 1H), 7.76 (s, 1H), 7.75-7.72 (m, 1H), 7.57 (s, 1H), 6.43 (s, 1H), 4.37-4.34 (m, 2H), 3.36-3.34 (m, 2H), 2.98 (s, 3H), 2.67 (s, 3H).

### Example 7: N-(5-(5-amino-1H-pyrazol-1-yl)-1,3,4-thiadiazol-2-yl)-4-(2-cyano-6-methoxyphenyl)-3-(2-methoxyethoxy)-2-oxo-2H-pyran-6-carboxamide (Compound 8) and N-(5-(5-acetamido-1H-pyrazol-1-yl)-1,3,4-thiadiazol-2-yl)-4-(2-cyano-6-methoxyphenyl)-3-(2-methoxyethoxy)-2-oxo-2H-pyran-6-carboxamide (Compound 8-Ac)

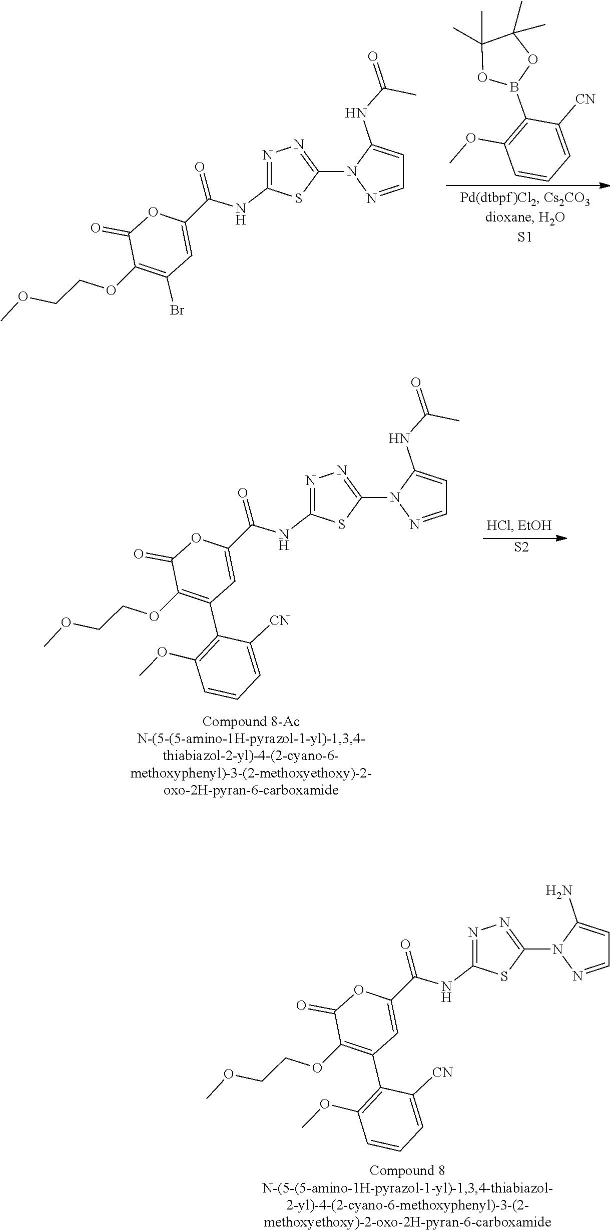

Step 1: Into a mixture of 3-methoxy-2-(4,4,5,5-tetramethyl-1,3,2-dioxaborolan-2-yl)benzonitrile (“aryl boron reagent”) (700 mg, 2.70 mmol, 1 equiv) in N,N-dimethylformamide (DMF) (10 mL) and H₂O (1 mL) was added 4-bromo-N-[5-(5-acetamidopyrazol-1-yl)-1,3,4-thiadiazol-2-yl]-5-(2-methoxyethoxy)-6-oxopyran-2-carboxamide (product of Step 4, Example 5, “halo-pyrone reagent”) (1.35 g, 2.70 mmol, 1 equiv), [1,1′-bis(di-tert-butylphosphino)ferrocene]dichloropalladium(II) (Pd(dtbpf)Cl₂) (352 mg, 0.54 inmol, 0.2 equiv) and K₃PO₄ (1.15 g, 5.40 mmol, 2 equiv) at room temperature. The resulting mixture was stirred for 3 h at 80° C. under nitrogen atmosphere. The mixture was then cooled to rt and diluted with ethyl acetate (EtOAc) (200 mL) then washed with water (3×10 mL) and then brine (1×20 mL). The organic extract was then dried over sodium sulfate and concentrated under reduced pressure and purified by silica gel column chromatography (eluting with CH₂Cl₂/MeOH (10:1)) followed by additional purification by C18 reverse phase flash chromatography (mobile phase, MeCN in water (10 mmol/L NH₄HCO₃), 10% to 50% gradient in 10 min; detector, UV 254 nm) to provide N-(5-(5-acetamido-1H-pyrazol-1-yl)-1,3,4-thiadiazol-2-yl)-4-(2-cyano-6-methoxyphenyl)-3-(2-methoxyethoxy)-2-oxo-2H-pyran-6-carboxamide (Compound 8-Ac) (120 mg, 8% yield). LCMS (ES, m/z)=552.05 [M+H]+.

Step 2: To a solution of N-(5-(5-acetamido-1H-pyrazol-1-yl)-1,3,4-thiadiazol-2-yl)-4-(2-cyano-6-methoxyphenyl)-3-(2-methoxyethoxy)-2-oxo-2H-pyran-6-carboxamide (Compound 8-Ac) (30 mg, 0.05 mmol, 1 equiv) in EtOH (1 mL) was added conc. HCl (1 mL) at 0° C. The resulting mixture was then stirred for 2 h at room temperature then concentrated under vacuum. The resulting residue was then purified by C18 reverse phase flash chromatography (mobile phase, MeCN in water (0.1% TFA), 10% to 50% gradient in 10 min; detector, UV 254 nm) to provide N-(5-(5-amino-1H-pyrazol-1-yl)-1,3,4-thiadiazol-2-yl)-4-(2-cyano-6-methoxyphenyl)-3-(2-methoxyethoxy)-2-oxo-2H-pyran-6-carboxamide (Compound 8) (1.7 mg, 5.9% yield). LCMS (ES, m/z)=510.30 [M+H]+; ¹H NMR (400 MHz, DMSO-d6) δ 7.67 (t, J=8.0 Hz, 1H), 7.56-7.54 (m, 2H), 7.48 (d, J=2.0 Hz, 1H), 7.44 (s, 1H), 6.65 (s, 2H), 5.45 (d, J=2.0 Hz, 1H), 4.34-4.27 (m, 2H), 3.87 (s, 3H), 3.51 (s, 2H), 3.04 (s, 3H).

### Example 8: N-(5-(5-amino-1H-pyrazol-1-yl)-1,3,4-thiadiazol-2-yl)-4-(3-cyanopyridin-2-yl)-3-(2-methoxyethoxy)-2-oxo-2H-pyran-6-carboxamide (Compound 9) and N-(5-(5-acetamido-1H-pyrazol-1-yl)-1,3,4-thiadiazol-2-yl)-4-(3-cyanopyridin-2-yl)-3-(2-methoxyethoxy)-2-oxo-2H-pyran-6-carboxamide (Compound 9-Ac)

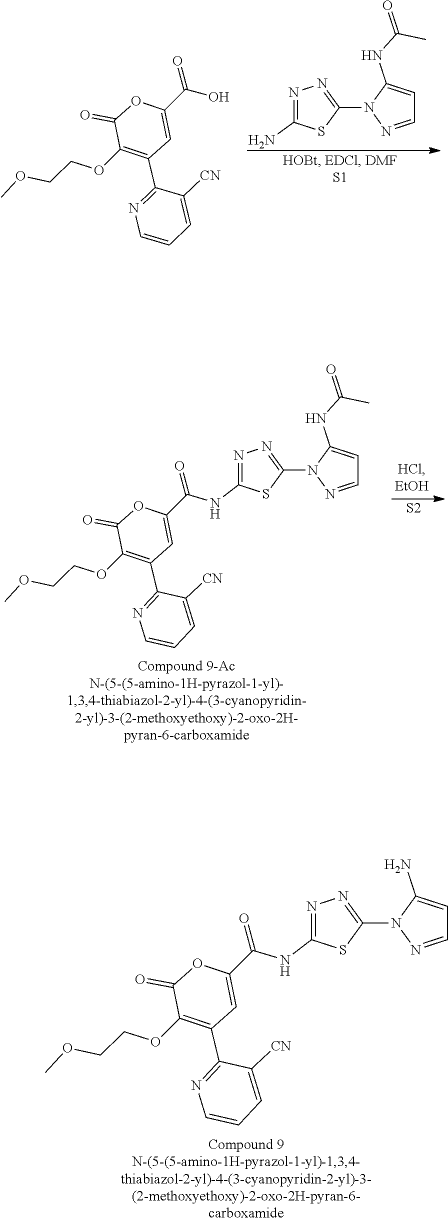

Step 1: To a stirred solution of 4-(3-cyanopyridin-2-yl)-5-(2-methoxyethoxy)-6-oxopyran-2-carboxylic acid (product of Step 3, Example 6; “aryl-pyrone reagent”) (100 mg, 0.32 mmol, 1 equiv) and N-(1-(5-amino-1,3,4-thiadiazol-2-yl)-1H-pyrazol-5-yl)acetamide (product of Step 3, Example 5; “ADT amine reagent”) (71 mg, 0.32 mmol, 1 equiv) in N,N-dimethylformamide (DMF) (1 mL) was added hydroxybenzotriazole (HOBT) (85 mg, 0.63 mmol, 2 equiv) and 1-ethyl-3-(3-dimethylaminopropyl)carbodiimide (EDCI) (182 mg, 0.95 mmol, 3 equiv) at room temperature. The resulting mixture was stirred for 1 h at room temperature then diluted with water (10 mL) and extracted with ethyl acetate (EtOAc) (3×20 mL). The combined organic layers were washed with water (3×2 mL) then with brine (10 mL), and dried over anhydrous Na₂SO₄. After filtration, the filtrate was concentrated under reduced pressure and the crude residue was purified by silica gel column chromatography, eluting with petroleum ether/ethyl acetate (PE/EtOAc) (1:5), to afford N-(5-(5-acetamido-1H-pyrazol-1-yl)-1,3,4-thiadiazol-2-yl)-4-(3-cyanopyridin-2-yl)-3-(2-methoxyethoxy)-2-oxo-2H-pyran-6-carboxamide (Compound 9-Ac) (80 mg, 46% yield). LCMS (ES, m/z)=523.20 [M+H]+.

Step 2: N-(5-(5-amino-1H-pyrazol-1-yl)-1,3,4-thiadiazol-2-yl)-4-(3-cyanopyridin-2-yl)-3-(2-methoxyethoxy)-2-oxo-2H-pyran-6-carboxamide (Compound 9) was prepared according to Step 2 of Example 7 by acidic deprotection of N-(5-(5-acetamido-1H-pyrazol-1-yl)-1,3,4-thiadiazol-2-yl)-4-(3-cyanopyridin-2-yl)-3-(2-methoxyethoxy)-2-oxo-2H-pyran-6-carboxamide (Compound 9-Ac). LCMS (ES, m/z)=481.05 [M+H]+; ¹H NMR (400 MHz, DMSO-d6) δ 13.46 (br, 1H), 9.03 (d, J=5.6 Hz, 1H), 8.51 (d, J=5.6 Hz, 1H), 7.75 (dd, J=8.0, 4.8 Hz, 1H), 7.61 (s, 1H), 7.48 (d, J=1.6 Hz, 1H), 6.65 (s, 2H), 5.45 (d, J=1.6 Hz, 1H), 4.41-4.34 (m, 2H), 3.37-3.32 (m, 2H), 2.98 (s, 3H).

### Example 9: N-(5-(5-amino-1H-pyrazol-1-yl)-1,3,4-thiadiazol-2-yl)-3-(2-methoxyethoxy)-4-(3-methoxypyridin-2-yl)-2-oxo-2H-pyran-6-carboxamide (Compound 10) and N-(5-(5-acetamido-1H-pyrazol-1-yl)-1,3,4-thiadiazol-2-yl)-3-(2-methoxyethoxy)-4-(3-methoxypyridin-2-yl)-2-oxo-2H-pyran-6-carboxamide (Compound 10-Ac)

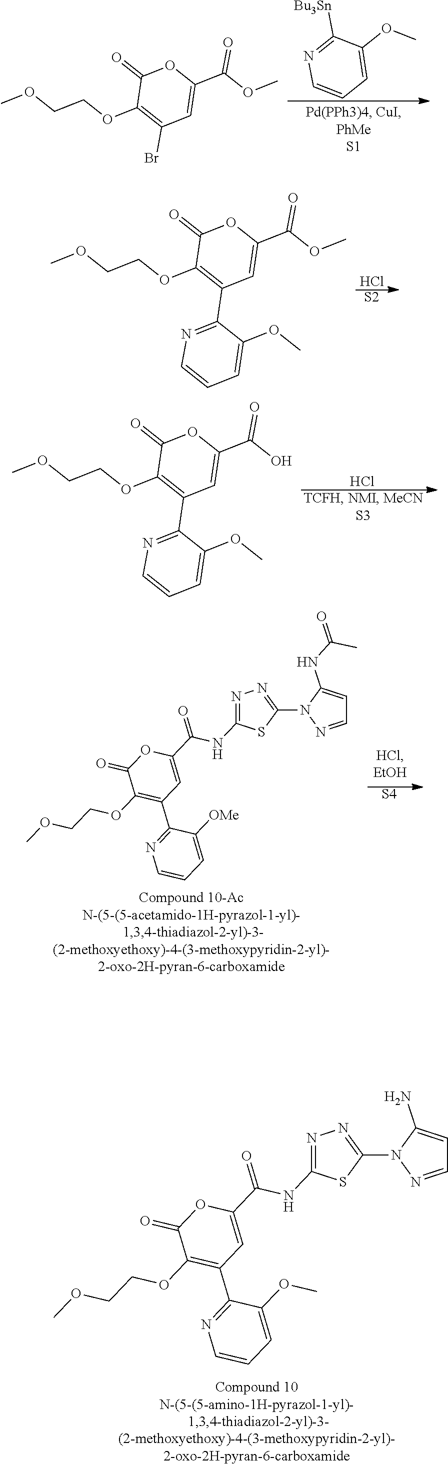

Step 1: To a stirred solution of methyl 4-bromo-3-(2-methoxyethoxy)-2-oxo-2H-pyran-6-carboxylate (product of Step 1, Example 3) (500 mg, 1.63 mmol, 1 equiv) in toluene (6 mL) was added tetrakis (triphenylphosphine)palladium (0) (Pd(PPh₃)₄) (376 mg, 0.32 mmol, 0.2 equiv), CuI (106 mg, 0.56 mmol, 0.34 equiv) and 3-methoxy-2-(tributylstannyl)pyridine (1.30 g, 3.27 mmol, 2 equiv) at room temperature under nitrogen atmosphere. The resulting mixture was stirred for 1 h at 100° C. The reaction was then cooled to rt and saturated KF (aq.) was added and extracted with ethyl acetate (EtOAc) (3×50 mL). The combined organic layers were washed with brine (20 mL), and dried over anhydrous Na₂SO₄. After filtration, the filtrate was concentrated under reduced pressure and the crude residue was purified by silica gel column chromatography (eluting with petroleum ether/ethyl acetate (PE/EtOAc) (1:1)) to afford methyl 3-(2-methoxyethoxy)-4-(3-methoxypyridin-2-yl)-2-oxo-2H-pyran-6-carboxylate (450 mg, 74% yield). LCMS (ES, m/z)=336.1[M+H]+.

Step 2: A solution of methyl 3-(2-methoxyethoxy)-4-(3-methoxypyridin-2-yl)-2-oxo-2H-pyran-6-carboxylate (400 mg, 1.19 mmol, 1 equiv) in hydrochloric acid (8 mL, 6 M) was stirred for 1 h at 80° C. then cooled to rt and concentrated under reduced pressure to afford crude 3-(2-methoxyethoxy)-4-(3-methoxypyridin-2-yl)-2-oxo-2H-pyran-6-carboxylic acid (390 mg, 100% yield) which was used directly in the next step without further purification. LCMS (ES, m/z)=322.1[M+H]+.

Step 3: To a solution of 3-(2-methoxyethoxy)-4-(3-methoxypyridin-2-yl)-2-oxo-2H-pyran-6-carboxylic acid (“aryl-pyrone reagent”) (320 mg, 1 mmol, 1 equiv) in MeCN (6 mL) was added chloro-N,N,N′,N′-tetramethylformamidinium hexafluorophosphate (TCFH) (545 mg, 1.94 mmol, 2 equiv) and N-methylimidazole (NMI) (320 mg, 3.89 mmol, 4 equiv) and N-(1-(5-amino-1,3,4-thiadiazol-2-yl)-1H-pyrazol-5-yl)acetamide (product of Step 3, Example 5; “ADT amine reagent”) (257 mg, 1.14 mmol, 1.2 equiv) at room temperature and then the resulting mixture was stirred for 1 h at room temperature. The reaction mixture was then filtered and the filter cake was washed with water (3×3 mL). The filtrate was concentrated under reduced pressure and the resulting residue was purified by C18 reverse phase flash chromatography (mobile phase, MeCN in water (0.1% NH₃·H₂O), 10% to 21% gradient in 10 min; detector, UV 254 nm) to provide N-(5-(5-acetamido-1H-pyrazol-1-yl)-1,3,4-thiadiazol-2-yl)-3-(2-methoxyethoxy)-4-(3-methoxypyridin-2-yl)-2-oxo-2H-pyran-6-carboxamide (Compound 10-Ac) (110 mg, 19% yield). LCMS (ES, m/z)=528.0 [M+1]+.

Step 4: To N-(5-(5-acetamido-1H-pyrazol-1-yl)-1,3,4-thiadiazol-2-yl)-3-(2-methoxyethoxy)-4-(3-methoxypyridin-2-yl)-2-oxo-2H-pyran-6-carboxamide (Compound 10-Ac) (50 mg, 0.076 mmol, 1 equiv) was added HCl (0.5 mL, 12 M) and ethanol (EtOH) (0.5 mL) at room temperature. The resulting mixture was stirred overnight at room temperature then concentrated under vacuum. The resulting residue was purified by C18 reverse phase flash chromatography (mobile phase, MeCN in water (10 mmol/L NH₄HCO₃), 10% to 20% gradient in 10 min; detector, UV 254 nm) to provide N-(5-(5-amino-1H-pyrazol-1-yl)-1,3,4-thiadiazol-2-yl)-3-(2-methoxyethoxy)-4-(3-methoxypyridin-2-yl)-2-oxo-2H-pyran-6-carboxamide (Compound 10) (18 mg, 47% yield). LCMS (ES, m/z)=486.05 [M+1]+; ¹H NMR (400 MHz, DMSO-d6) δ 3.38 (s, 1H), 8.31-8.30 (m, 1H), 7.70-7.65 (s, 1H), 7.53-7.45 (m, 3H), 6.66 (s, 2H), 5.44 (s, 1H), 4.27-4.26 (m, 2H), 3.96-3.89 (m, 3H), 3.59 (s, 2H), 3.18-3.08 (m, 3H).

### Example 10: N-(5-(5-amino-1H-pyrazol-1-yl)-1,3,4-thiadiazol-2-yl)-4-(2,6-dicyanophenyl)-3-(2-methoxyethoxy)-2-oxo-2H-pyran-6-carboxamide (Compound 12) and N-(5-(5-acetamido-1H-pyrazol-1-yl)-1,3,4-thiadiazol-2-yl)-4-(2,6-dicyanophenyl)-3-(2-methoxyethoxy)-2-oxo-2H-pyran-6-carboxamide (Compound 12-Ac)

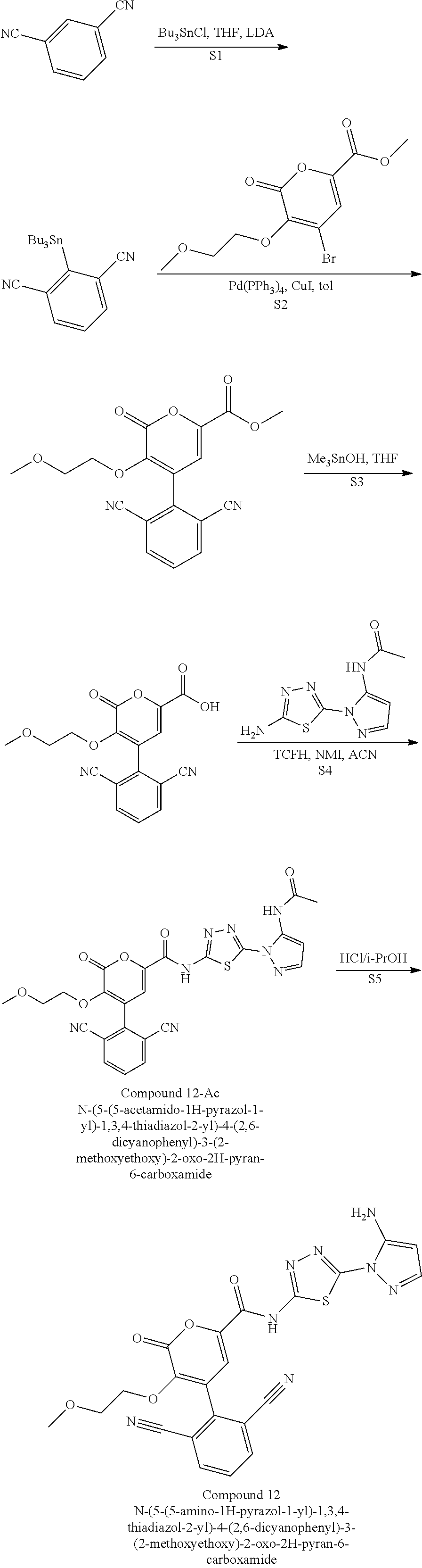

Step 1: To a solution of isophthalonitrile (2 g, 16 mmol, 1 equiv) in tetrahydrofuran (THF) (100 mL) was added a solution of lithium diisopropyl amide (LDA) (12.8 mL of a 2M solution in n-hexane, 25.6 mmol, 1.6 equiv) in n-hexane dropwise at −78° C. under nitrogen atmosphere. The resulting mixture was stirred for 0.5 h at −78° C. then tributyltin chloride (Bu₃SnCl) (6.7 g, 21 mmol, 1.3 equiv) was added at −78° C. The resulting mixture was stirred for additional 3 h at −78° C. before being poured into water (100 mL) and extracted with ethyl acetate (EtOAc) (3×200 mL). The combined organic layers were washed with water (300 mL), and dried over anhydrous Na₂SO₄. After filtration, the filtrate was concentrated under reduced pressure and the residue was purified by silica gel column chromatography (eluting with PE/EA (1:1)) to afford 2-(tributylstannyl)isophthalonitrile (4.5 g, 69% yield). ¹H NMR (300 MHz, Chloroform-d) δ 7.87-7.81 (m, 2H), 7.54-7.48 (m, 1H), 1.74-1.49 (m, 6H), 1.49-1.22 (m, 12H), 1.00-0.82 (m, 9H).

Step 2: To a stirred solution of methyl 4-bromo-3-(2-methoxyethoxy)-2-oxo-2H-pyran-6-carboxylate (product of Step 1, Example 3) (3 g, 9.8 mmol, 1 equiv) and 2-(tributylstannyl)isophthalonitrile (6.12 g, 14.7 mmol, 1.50 equiv) in toluene (30 mL) was added tetrakis (triphenylphosphine)palladium (0) (Pd(PPh₃)₄) (2.3 g, 2.0 mmol, 0.2 equiv) and CuI (0.95 g, 5.0 mmol, 0.51 equiv) at room temperature. The resulting mixture was stirred for 2 h at 100° C. under nitrogen atmosphere, then cooled to rt, filtered, and concentrated under reduced pressure. The residue obtained was then purified by C18 reverse phase flash chromatography (mobile phase. MeCN in water (5 mmol/L NH₄HCO₃), 0% to 100% gradient in 10 min; detector, UV 254 nm) to provide methyl 4-(2,6-dicyanophenyl)-3-(2-methoxyethoxy)-2-oxo-2H-pyran-6-carboxylate (1.7 g, 25% yield). LCMS (ES, m/z)=355.1 [M+H]+.

Step 3: To a solution of methyl 4-(2,6-dicyanophenyl)-3-(2-methoxyethoxy)-2-oxo-2H-pyran-6-carboxylate (1.7 g, 4.8 mmol, 1 equiv) in tetrahydrofuran (THF) (10 mL) was added trimethylstannanol (0.9 g, 5.0 mmol, 1 equiv) at room temperature. The resulting mixture was stirred overnight at room temperature then poured into water and acidified to pH 5 with HCl (aq.). The resulting mixture was extracted with ethyl acetate (EtOAc) (3×100 mL) and the combined organic layers were washed with water (50 mL) then brine (50 mL), and dried over anhydrous Na₂SO₄. After filtration, the filtrate was concentrated under reduced pressure and the residue obtained was purified by C18 reverse phase flash chromatography (mobile phase, MeCN in water (5 mmol/L NH₄HCO₃), 0% to 0% gradient in 10 min; detector, UV 254 nm) to provide 4-(2,6-dicyanophenyl)-3-(2-methoxyethoxy)-2-oxo-2H-pyran-6-carboxylic acid (320 mg, 20% yield). LCMS (ES, m/z)=341.1 [M+H]+.

Step 4: N-(5-(5-acetamido-1H-pyrazol-1-yl)-1,3,4-thiadiazol-2-yl)-4-(2,6-dicyanophenyl)-3-(2-methoxyethoxy)-2-oxo-2H-pyran-6-carboxamide (Compound 12-Ac) was prepared according to Step 3 of Example 9 using 4-(2,6-dicyanophenyl)-3-(2-methoxyethoxy)-2-oxo-2H-pyran-6-carboxylic acid in place of 3-(2-methoxyethoxy)-4-(3-methoxypyridin-2-yl)-2-oxo-2H-pyran-6-carboxylic acid as the “aryl-pyrone reagent” and using N-(1-(5-amino-1,3,4-thiadiazol-2-yl)-1H-pyrazol-5-yl)acetamide (product of Step 3, Example 5; “ADT amine reagent”). LCMS (ES, m/z)=547 [M+H]+.

Step 5: N-(5-(5-amino-1H-pyrazol-1-yl)-1,3,4-thiadiazol-2-yl)-4-(2,6-dicyanophenyl)-3-(2-methoxyethoxy)-2-oxo-2H-pyran-6-carboxamide (Compound 12) was prepared following Example 9 Step 4 by acidic deprotection of N-(5-(5-acetamido-1H-pyrazol-1-yl)-1,3,4-thiadiazol-2-yl)-4-(2,6-dicyanophenyl)-3-(2-methoxyethoxy)-2-oxo-2H-pyran-6-carboxamide (Compound 12-Ac). LCMS (ES, m/z)=505.0 [M+1]+; ¹H NMR (400 MHz, DMSO-d6) δ 13.55 (s, 1H), 8.39 (d, J=7.6 Hz, 2H), 7.91 (dd, J=8.0, 2.0 Hz, 1H), 7.70 (s, 1H), 7.48 (s, 1H), 6.66 (s, 2H), 5.45 (s, 1H), 4.50-4.32 (m, 2H), 3.34-3.33 (m, 2H), 3.01 (s, 3H).

### Example 11: 4-(4-cyano-1-methyl-1H-pyrazol-5-yl)-3-methoxy-N-(5-(5-methyl-1H-pyrazol-1-yl)-1,3,4-thiadiazol-2-yl)-2-oxo-2H-pyran-6-carboxamide (Compound 14)

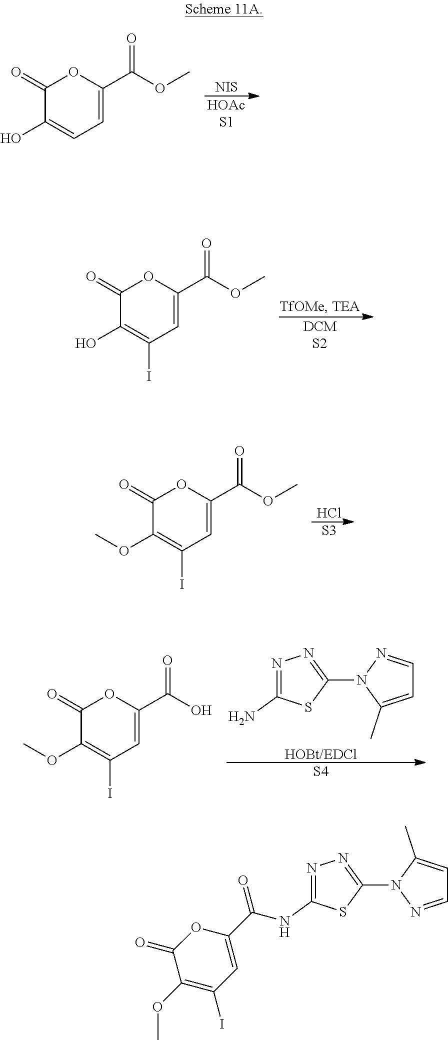

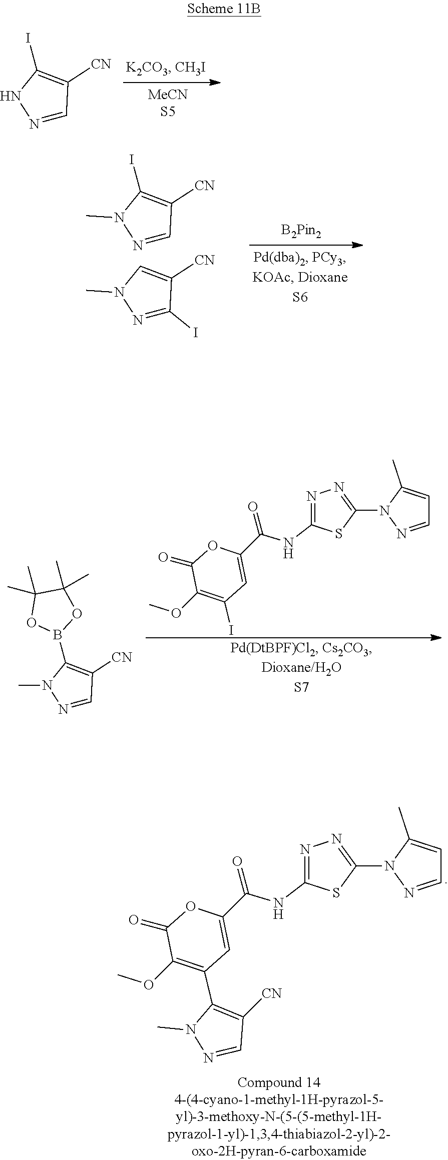

Step 1: Into a solution of methyl 5-hydroxy-6-oxopyran-2-carboxylate (25 g, 147 mmol, 1 equiv) in acetic acid (AcOH) (300 mL) was added N-iodosuccinimide (NIS) (39 g, 173 mmol, 1.18 equiv) in portions at room temperature. The resulting mixture was stirred for 20 h at 80° C. then concentrated under reduced pressure. The residue was dissolved in ethyl acetate (EtOAc) (1 L) and washed with water (3×100 mL), and dried over anhydrous Na₂SO₄. After filtration, the filtrate was concentrated under reduced pressure. The residue was purified using silica gel column chromatography, eluting with petroleum ether/ethyl acetate (PE/EtOAc) (3:2), to afford methyl 5-hydroxy-4-iodo-6-oxopyran-2-carboxylate (20 g, 46% yield). LCMS (ESI, m/z)=295 [M−1]−.

Step 2: Into a solution of methyl 5-hydroxy-4-iodo-6-oxopyran-2-carboxylate (20 g, 67.6 mmol, 1 equiv) in dichloromethane (DCM) (250 mL) was added diisopropylethylamine (DIEA) (26 g, 201 mmol, 3 equiv) at room temperature. To the above mixture was added triflate ester (33 g, 201 mmol, 3 equiv) dropwise at 0° C. The resulting mixture was stirred overnight at room temperature then poured into water and extracted with DCM (3×500 mL). The combined organic layers were washed with water (3×100 mL), and dried over anhydrous Na₂SO₄. After filtration, the filtrate was concentrated under reduced pressure and the residue was purified by silica gel column chromatography, eluting with petroleum ether/ethyl acetate (PE/EtOAc) (4:1), to afford methyl 4-iodo-5-methoxy-6-oxopyran-2-carboxylate (19 g, 91% yield). LCMS (ESI, m/z)=311 [M+1]+.

Step 3: A solution of methyl 4-iodo-5-methoxy-6-oxopyran-2-carboxylate (5 g, 16 mmol, 1 equiv) in HCl (6M) (100 mL) was stirred for 4 h at 80° C. The resulting mixture was then concentrated under reduced pressure to provide 4-iodo-5-methoxy-6-oxopyran-2-carboxylic acid (3.8 g, 80% yield). LCMS (ESI, m/z)=295 [M−1]−.

Step 4: A solution of 4-iodo-5-methoxy-6-oxopyran-2-carboxylic acid (2.60 g, 8.78 mmol, 1 equiv), hydroxybenzotriazole (HOBT) (1.80 g, 13.3 mmol, 1.5 equiv), 1-ethyl-3-(3-dimethylaminopropyl)carbodiimide (EDCI) (3.60 g, 18.8 mmol, 2.1 equiv) and 5-(5-methylpyrazol-1-yl)-1,3,4-thiadiazol-2-amine (product of Step 8, Example 1) (1.40 g, 7.72 mmol, 0.9 equiv) in N,N-dimethylformamide (DMF) (40 mL) was stirred for 1 h at room temperature. The reaction was quenched by the addition of water (20 mL). The precipitated solids were collected by filtration and washed with acetonitrile (MeCN) (5×1 mL) to provide 4-iodo-3-methoxy-N-(5-(5-methyl-1H-pyrazol-1-yl)-1,3,4-thiadiazol-2-yl)-2-oxo-2H-pyran-6-carboxamide (3.1 g, 77% yield). LCMS (ESI, m/z)=460 [M+1]+.

Step 5: To a solution of 3-iodo-2H-pyrazole-4-carbonitrile (1 g, 4.6 mmol, 1 equiv) and K₂CO₃ (1.26 g, 9.13 mmol, 2 equiv) in acetonitrile (MeCN) (12 mL) was added methyl iodide (MeI) (1.94 g, 13.7 mmol, 3 equiv) dropwise at room temperature and then the resulting solution was stirred for 1 h at room temperature. The mixture was then diluted with water (40 mL) and extracted with ethyl acetate (EtOAc) (4×30 mL). The combined organic layers were dried over anhydrous Na₂SO₄, filtered, and concentrated under reduced pressure. The resulting residue was purified by silica gel column chromatography, eluting with petroleum ether/ethyl acetate (PE/EtOAc) (6:1), to afford 5-iodo-1-methylpyrazole-4-carbonitrile (540 mg, 34% yield) and 3-iodo-1-methyl-1H-pyrazole-4-carbonitrile (340 mg, 21% yield). LCMS (ES, m/z)=234.1 [M+H]+.

Step 6: A solution of tricyclohexylphospine (PCy₃) (130 mg, 0.46 mmol, 0.2 equiv) and bis (dibenzylideneacetone)palladium(0) (Pd(dba)₂) (280 mg, 0.49 mmol, 0.21 equiv) in 1,4-dioxane (5 mL) was stirred for 30 min at room temperature under nitrogen atmosphere. To the above mixture was then added 5-iodo-1-methylpyrazole-4-carbonitrile (540 mg, 2.32 mmol, 1 equiv), bis(pinacolato)diboron (880 mg, 3.46 mmol, 1.5 equiv) and potassium acetate (KOAc) (486 mg, 4.95 mmol, 2 equiv) under nitrogen, and then the resulting solution was stirred for 1 h at 120° C. under nitrogen atmosphere. The mixture was then cooled to rt and diluted with water (20 mL), then extracted with ethyl acetate (EtOAc) (3×50 mL). The combined organic layers were dried over anhydrous Na₂SO₄, filtered, and concentrated under reduced pressure to provide 1-methyl-5-(4,4,5,5-tetramethyl-1,3,2-dioxaborolan-2-yl)pyrazole-4-carbonitrile (500 mg, 93% yield) which was used directly in the next step without further purification. LCMS (ES, m/z)=234.1 [M+H]+.

Step 7: A solution of 1-methyl-5-(4,4,5,5-tetramethyl-1,3,2-dioxaborolan-2-yl)pyrazole-4-carbonitrile (“aryl boron reagent”) (500 mg, 2.14 mmol, 1 equiv), [1,1′-bis(di-tert-butylphosphino)ferrocene]dichloropalladium(II) (Pd(DtBPF)Cl₂) (285 mg, 0.44 mmol, 0.2 equiv), Cs₂CO₃ (1.40 g, 4.29 mmol, 2 equiv) and 4-iodo-3-methoxy-N-(5-(5-methyl-1H-pyrazol-1-yl)-1,3,4-thiadiazol-2-yl)-2-oxo-2H-pyran-6-carboxamide (product of Step 4, Example 11; “halo-pyrone reagent”) (800 mg, 1.74 mmol, 0.8 equiv) in a mixture of 1,4-dioxane/H₂O (5 mL, 6:1) was stirred for 1 h at 80° C. under nitrogen atmosphere. The mixture was then concentrated under vacuum and the residue was purified by silica gel column chromatography (eluting with CH₂Cl₂/methanol (MeOH) (10:1)) followed by additional purification by Prep-HPLC (XBridge Prep OBD C18 Column, 30*150 mm; Mobile Phase A: water (10 mmol/L NH₄HCO₃), Mobile Phase B: acetonitrile (MeCN); Flow rate: 60 mL/min: Gradient: 12% B to 22% B in 10 min; Wave Length: 220/254 nm) to afford 4-(4-cyano-1-methyl-1H-pyrazol-5-yl)-3-methoxy-N-(5-(5-methyl-1H-pyrazol-1-yl)-1,3,4-thiadiazol-2-yl)-2-oxo-2H-pyran-6-carboxamide (Compound 14) (1.5 mg, 0.2% yield). LCMS (ES, m/z)=439.0 [M+H]+; ¹H NMR (300 MHz, DMSO-d6) δ 8.70 (s, 1H), 7.62 (d, J=1.5 Hz, 1H), 7.54 (s, 1H), 6.30 (d, J=1.5 Hz, 1H), 4.03 (s, 3H), 4.00 (s, 3H), 2.60 (s, 3H).

### Example 12: (Ra)—N-(5-(5-amino-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15A*), (Sa)-N-(5-(5-amino-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15B*), and N-(5-(5-acetamido-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15-Ac)

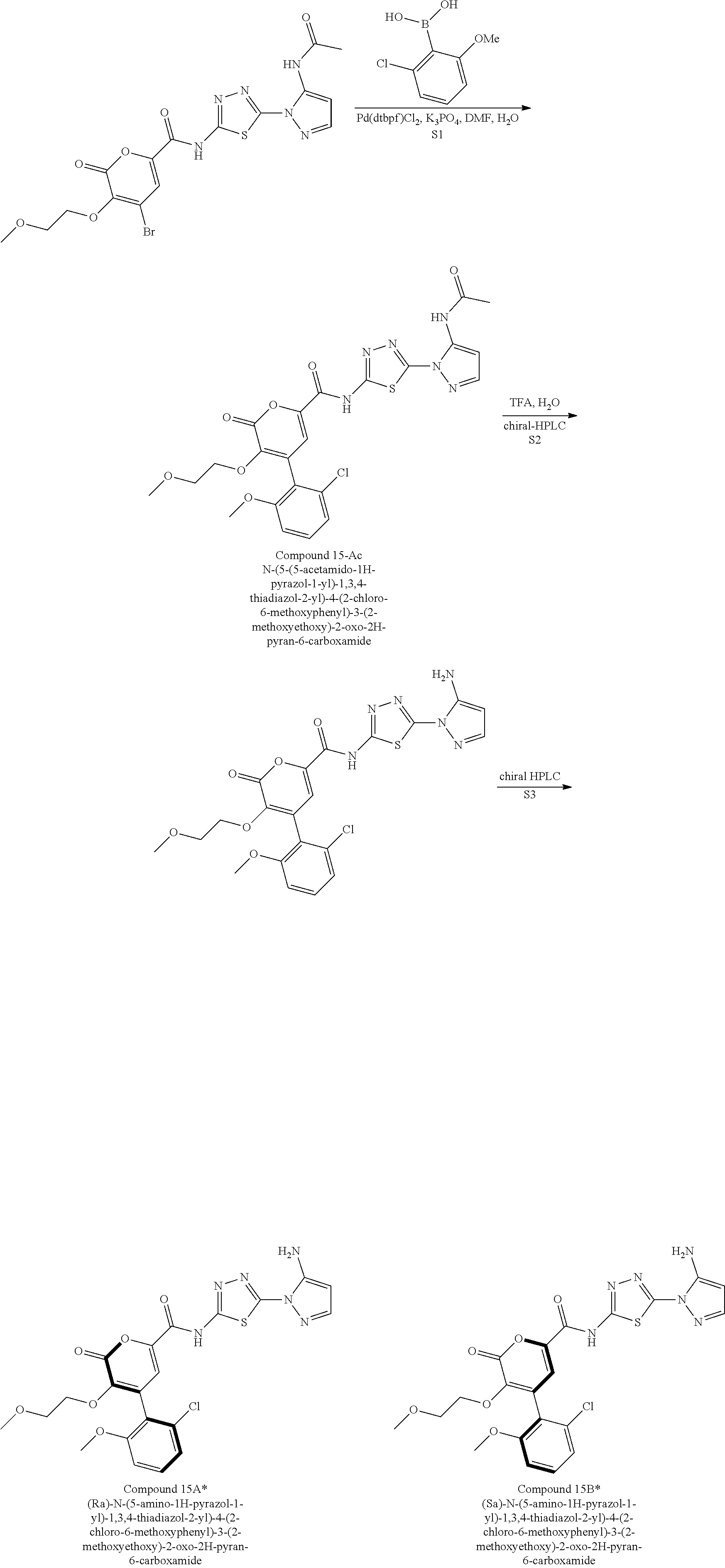

Step 1: A mixture of 4-bromo-N-[5-(5-acetamidopyrazol-1-yl)-1,3,4-thiadiazol-2-yl]-5-(2-methoxyethoxy)-6-oxopyran-2-carboxamide (product of Step 4, Example 5; “halo-pyrone reagent”) (365 mg, 0.73 mmol, 1 equiv), [1,1′-bis(di-tert-butylphosphino)ferrocene]dichloropalladium(II) (Pd(dthpf)Cl₂) (95 mg, 0.15 mmol, 0.20 equiv), K₃PO₄ (466 mg, 2.20 mmol, 3 equiv) and 2-chloro-6-methoxyphenylboronic acid (“aryl boron reagent”) (822 mg, 4.41 mmol, 6 equiv) in N,N-dimethylformamide (DMF) (15.2 mL) and water (1.68 mL) was stirred overnight at 100° C. under nitrogen atmosphere. The mixture was then cooled to it and diluted with water (30 mL). The mixture was then extracted with ethyl acetate (EtOAc) (3×50 mL), and the organic layers were combined and washed with water (3×10 mL), dried over anhydrous Na₂SO₄, filtered, and concentrated under reduced pressure. The resulting residue was then purified by silica gel column chromatography (eluting with petroleum ether/ethyl acetate (PE/EtOAc) (1:1)) to afford N-(5-(5-acetamido-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15-Ac) (82 mg, 17% yield). LCMS (ES, m/z)=561.1[M+H]+.

Steps 2-3: To a solution of N-(5-(5-acetamido-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15-Ac) (300 mg, 0.54 mmol, 1 equiv) in tetrahydrofuran (THF) (I mL) and water (1 mL) was added trifluoroacetic acid (TFA) (4 mL). The resulting solution was stirred for 3 h at 80° C. under nitrogen atmosphere. The mixture was then cooled to rt and diluted with water (50 mL) and extracted with ethyl acetate (EtOAc) (3×50 mL). The combined organic layers were then dried over anhydrous Na₂SO₄, filtered, and concentrated under reduced pressure. The crude material was purified by Prep-HPLC (Xselect CSH C18 OBD Column 30*150 mm; Mobile Phase A: water (10 mmol/L NH₄HCO₃), Mobile Phase B: acetonitrile (MeCN); Flow rate: 60 mL/min; Gradient: 18% B to 28% B in 10 min, 28% B to 28% B in 12 min, 28% B; Wave Length: 254/220 nm; RT(min): 11.670) followed by separation of axially chiral atropisomers by Prep-chiral-HPLC (CHIRALPAK IE-3, 4.6*50 mm 3 um; Mobile Phase A: Hexanes (0.1% TFA):(MeOH:DCM=1:1)=5:95; Flow rate: 1 mL/min; Gradient: 0% B to 0% B) followed by additional Prep Chiral HPLC (CHIRALPAK IE, 2*25 cm; Mobile Phase A: Hexanes (0.1% TFA), Mobile Phase B: MeOH:DCM=1:1; Flow rate: 20 mL/min; Gradient: isocratic; Wave Length: 220/254 nm) to afford (Ra)—N-(5-(5-amino-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15A*) (2.0 mg, 0.7% yield), as the first eluting peak (chiral RT=6.42 min), and (Sa)-N-(5-(5-amino-1H-pyrazol-1-yl)-1,3,4-thiadiazol-2-yl)-4-(2-chloro-6-methoxyphenyl)-3-(2-methoxyethoxy)-2-oxo-2H-pyran-6-carboxamide (Compound 15B*) (2.9 mg, 1% yield), as the second eluting peak (chiral RT=8.86 min). Stereochemistry arbitrarily assigned.

Compound 15A*: LCMS (ES, m/z)=519.0 [M+H]+, ¹H NMR (400 MHz, DMSO-d6) δ 13.33 (s, 1H), 7.58 (s, 2H), 7.48 (s, 1H), 7.27-7.16 (m, 2H), 6.67 (s, 2H), 5.46 (s, 1H), 4.30-4.27 (m, 2H), 3.81 (s, 3H), 3.51-3.48 (m, 2H), 3.07 (s, 3H)

Compound 15B*: LCMS (ES, m/z)=519.0 [M+H]+, ¹H NMR (400 MHz, DMSO-d6) δ 13.33 (s, 1H), 7.58 (s, 2H), 7.48 (s, 1H), 7.27-7.16 (m, 2H), 6.67 (s, 2H), 5.46 (s, 1H), 4.30-4.27 (m, 2H), 3.81 (s, 3H), 3.51-3.48 (m, 2H), 3.07 (s, 3H).

### Example 13: N-(5-(5-amino-1H-pyrazol-1-yl)-1,3,4-thiadiazol-2-yl)-4-(2,6-dimethoxyphenyl)-3-(2-hydroxyethoxy)-2-oxo-2H-pyran-6-carboxamide (Compound 17) and N-(5-(5-acetamido-1H-pyrazol-1-yl)-1,3,4-thiadiazol-2-yl)-3-(2-(tert-butoxy)ethoxy)-4-(2,6-dimethoxyphenyl)-2-oxo-2H-pyran-6-carboxamide (Compound 17-Ac-OtBu)

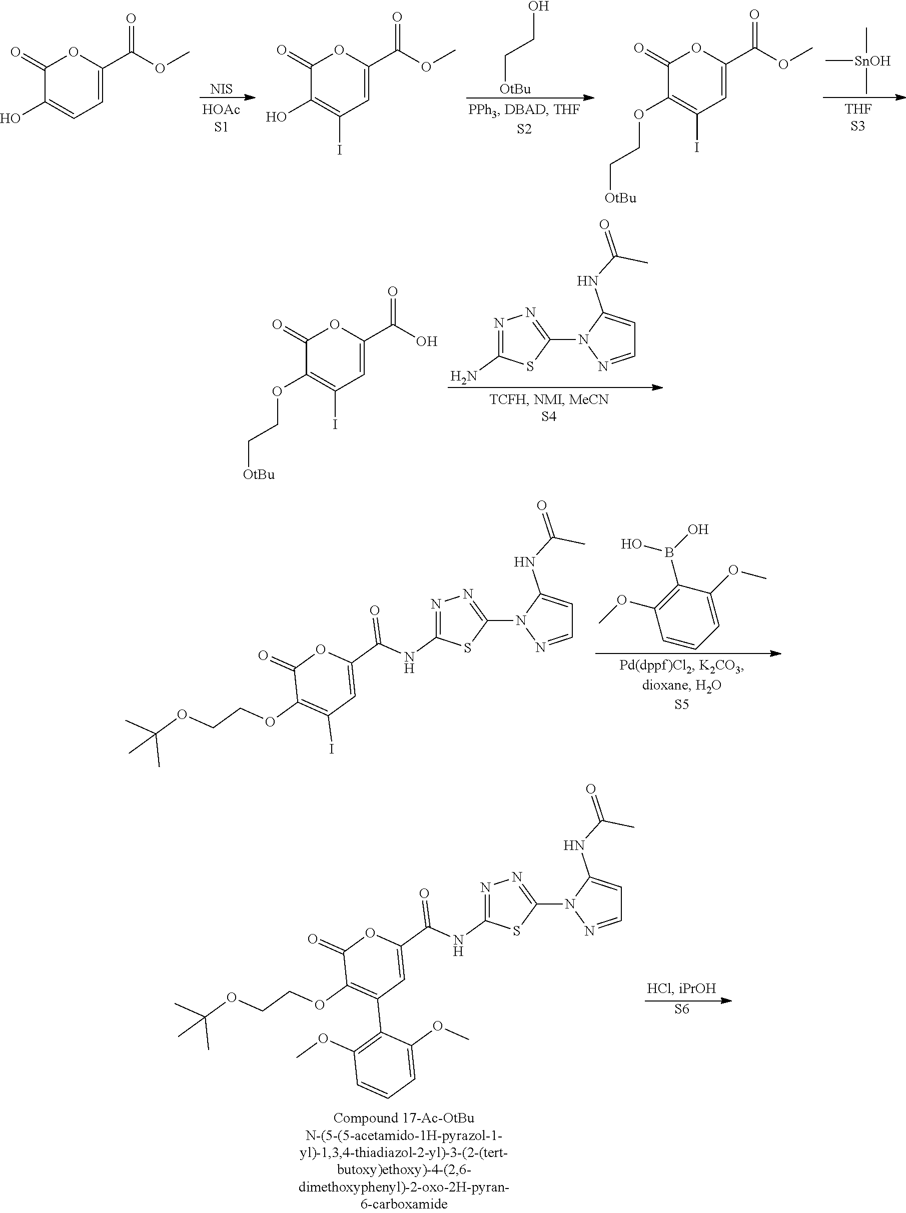

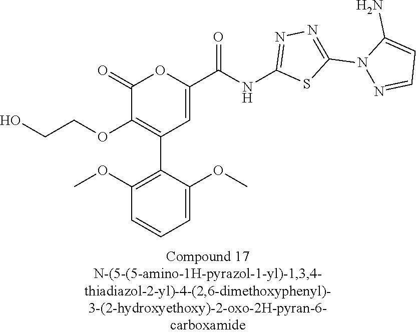 [SMILES: COc1cccc(OC)c1-c1cc(C(=O)Nc2nnc(-n3nccc3N)s2)oc(=O)c1OCCO]

Step 1: Methyl 5-hydroxy-4-iodo-6-oxopyran-2-carboxylate was synthesized according to Step 1 of Example 11. LCMS (ESI, m/z)=295 [M−1]−.

Step 2: To a stirred mixture of methyl 5-hydroxy-4-iodo-6-oxopyran-2-carboxylate (1000 mg, 3.378 mmol, 10 equiv) and triphenyl phosphine (PPh₃) (1328 mg, 5.063 mmol, 1.500 equiv) in tetrahydrofuran (THF) (15 mL) was added di-tert-butyl azodicarboxylate (DBAD) (1166 mg, 5.064 mmol, 1.500 equiv) in portions at 0° C. To the above mixture was added 2-(t-butoxy)ethanol (478 mg, 4.04 mmol, 1.20 equiv) in portions at 0° C. The resulting mixture was then stirred overnight at room temperature. The reaction was then quenched by the addition of water (50 mL) and the mixture was extracted with ethyl acetate (EtOAc) (3×50 mL). The combined organic layers were washed with brine (10 mL), and dried over anhydrous Na₂SO₄. After filtration, the filtrate was concentrated under reduced pressure and the residue was purified by silica gel column chromatography (eluting with petroleum ether/ethyl acetate (PE/EtOAc) (10:1)) to afford methyl 5-[2-(tert-butoxy)ethoxy]-4-iodo-6-oxopyran-2-carboxylate (1 g, 75% yield).

Step 3: Into a solution of methyl 5-[2-(tert-butoxy)ethoxy]-4-iodo-6-oxopyran-2-carboxylate (2 g, 5.0 mmol, 1 equiv) in tetrahydrofuran (THF) (5 mL) was added trimethyltin hydroxide (990 mg, 5.48 mmol, 1.08 equiv) at room temperature. The resulting mixture was stirred for 16 h at room temperature then concentrated under reduced pressure. The resulting residue was purified by C18 reverse phase flash chromatography (mobile phase, MeCN in water, 10% to 20% gradient in 10 min; detector, UV 254 nm) to provide 5-[2-(tert-butoxy)ethoxy]-4-iodo-6-oxopyran-2-carboxylic acid (1.73 g, 81% yield).

Step 4: A solution of 5-[2-(tert-butoxy)ethoxy]-4-iodo-6-oxopyran-2-carboxylic acid (289 mg, 0.76 mmol, 1 equiv) in MeCN (5 mL) was added N-methylimidazole (NMI) (186 mg, 2.27 mmol, 3 equiv), N-(1-(5-amino-1,3,4-thiadiazol-2-yl)-1H-pyrazol-5-yl)acetamide (product of Step 3, Example 5)(170 mg, 0.76 mmol, 1 equiv) and chloro-N,N,N,N-tetramethylformamidinium hexafluorophosphate (TCFH) (110 mg, 0.39 mmol, 1.50 equiv). The resulting mixture was stirred for 1 h at room temperature under nitrogen atmosphere. The mixture was then concentrated under reduced pressure and the resulting residue was purified by C18 reverse phase flash chromatography (mobile phase, MeCN in water (10 mmol/L NH₄HCO₃), 0% to 28% gradient in 10 min; detector, UV 254 nm) to provide 5-[2-(tert-butoxy)ethoxy]-N-[5-(5-acetamidopyrazol-1-yl)-1,3,4-thiadiazol-2-yl]-4-iodo-6-oxopyran-2-carboxamide (90 mg, 20% yield). LCMS (ES, m/z)=589.1 [M+H]+.

Step 5: A solution of 5-[2-(tert-butoxy)ethoxy]-N-[5-(5-acetamidopyrazol-1-yl)-1,3,4-thiadiazol-2-yl]-4-iodo-6-oxopyran-2-carboxamide (“halo-pyrone reagent”) (159 mg, 0.27 mmol, 1 equiv), [1,1′-bis(diphenylphosphino)ferrocene]dichloro palladium(II) (Pd(dppf)Cl₂)CH₂Cl₂ complex (44 mg, 0.054 mmol, 0.20 equiv), K₃PO₄ (172 mg, 0.81 mmol, 3 equiv) and 2,6-dimethoxyphenylboronic acid (“aryl boron reagent”) (98 mg, 0.54 mmol, 2 equiv) in a mixture solution of 1,4-dioxane (5 mL) and H₂O (1 mL) was stirred for 1 h at 70° C. under nitrogen atmosphere. The mixture was then cooled to rt and concentrated under reduced pressure and the residue was purified by C18 reverse phase flash chromatography (mobile phase, MeCN in water (0.1% FA), 0% to 60% gradient in 10 min; detector, UV 254 nm) to provide N-(5-(5-acetamido-1H-pyrazol-1-yl)-1,3,4-thiadiazol-2-yl)-3-(2-(tert-butoxy)ethoxy)-4-(2,6-dimethoxyphenyl)-2-oxo-2H-pyran-6-carboxamide (Compound 17-Ac-OtBu) (84 mg, 52% yield). LCMS (ES, m/z)=599.1 [M+H]+.

Step 6: To a solution of N-(5-(5-acetamido-1H-pyrazol-1-yl)-1,3,4-thiadiazol-2-yl)-3-(2-(tert-butoxy)ethoxy)-4-(2,6-dimethoxyphenyl)-2-oxo-2H-pyran-6-carboxamide (Compound 17-Ac-OtBu) (100 mg, 0.17 mmol, 1 equiv) in isopropanol (iPrOH) (6 mL) was added conc. HCl (1 mL). The resulting solution was stirred for 5 h at 60° C. then cooled to rt and concentrated under reduced pressure. The crude material was purified by Prep-HPLC (C18; mobile phase, methanol (MeOH) in water (0.1% HCl), 0% to 50% gradient in 20 min; detector. UV 254/nm) to afford N-(5-(5-amino-1H-pyrazol-1-yl)-1,3,4-thiadiazol-2-yl)-4-(2,6-dimethoxyphenyl)-3-(2-hydroxyethoxy)-2-oxo-2H-pyran-6-carboxamide (Compound 17) (22 mg, 26% yield). LCMS (ES, m/z)=501.1 [M+H]+: ¹H NMR (300 MHz, DMSO-d6) δ 13.21 (s, 1H), 7.47 (d, J=1.8 Hz, 1H), 7.41 (d, J=8.4 Hz, 1H), 7.27 (s, 1H), 6.78 (s, 1H), 6.76 (s, 1H), 6.65 (s, 2H), 5.43 (d, J=1.8 Hz, 1H), 4.63 (br, 1H), 4.06 (t, J=5.4 Hz, 2H), 3.75 (s, 6H), 3.45 (t, J=5.4 Hz, 2H).

### Example 14: N-(5-(3-fluoro-1-methyl-1H-pyrrol-2-yl)-1,3,4-thiadiazol-2-yl)-3-(2-methoxyethoxy)-4-(3-methoxypyridin-2-yl)-2-oxo-2H-pyran-6-carboxamide (Compound 18)

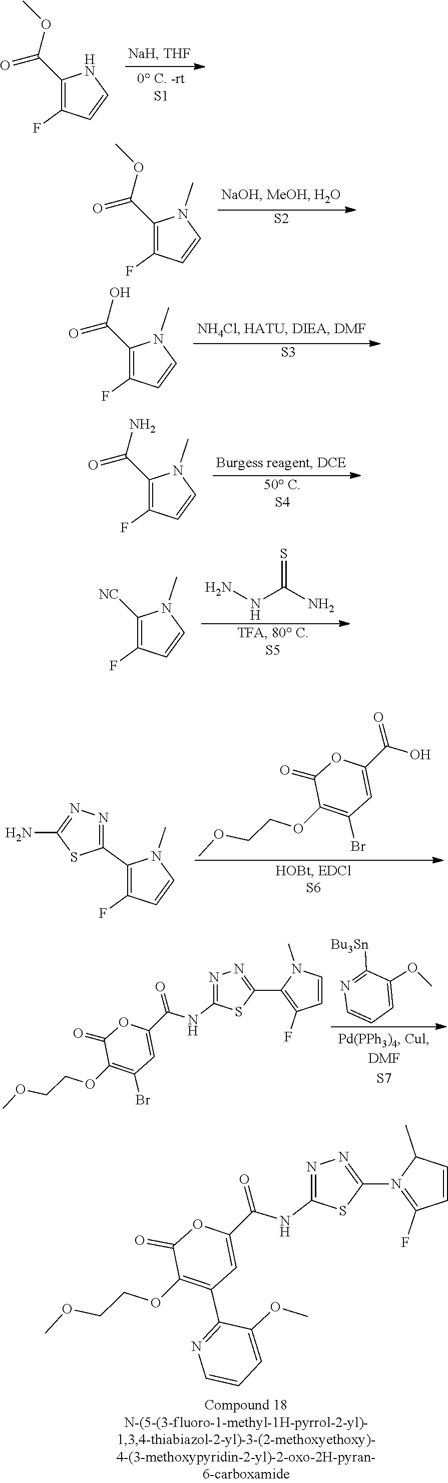

Steps 1-6:4-bromo-N-(5-(3-fluoro-1-methyl-1H-pyrrol-2-yl)-1,3,4-thiadiazol-2-yl)-3-(2-methoxyethoxy)-2-oxo-2H-pyran-6-carboxamide was prepared according to Steps 1-6 of Example 4 using methyl 3-fluoro-1H-pyrrole-2-carboxylate acid in place of methyl 3-chloro-1H-pyrrole-2-carboxylate. LCMS (ESI, m/z)=473.1 [M+H]+.

Step 7: To a stirred solution of 4-bromo-N-[5-(3-fluoro-1-methylpyrrol-2-yl)-1,3,4-thiadiazol-2-yl]-5-(2-methoxyethoxy)-6-oxopyran-2-carboxamide (“halo-pyrone reagent”) (40 mg, 0.085 mmol, 1 equiv) and 3-methoxy-2-(tributylstannyl)pyridine (“aryl tin reagent”) (150 mg, 0.38 mmol, 4.5 equiv) in N,N-dimethylformamide (DMF) (1 mL) was added tetrakis (triphenylphosphine)palladium (0) (Pd(PPh₃)₄) (30 mg, 0.026 mmol, 0.3 equiv) and CuI (7 mg, 0.04 mmol, 0.4 equiv) at room temperature. The resulting mixture was stirred for 3 h at 100° C. under nitrogen atmosphere then cooled to rt and directly purified by C18 reverse phase flash chromatography (mobile phase, MeCN in water (0.1% TFA), 20% to 40% gradient in 15 min; detector, UV 254 nm) to provide a residue which was then triturated with acetonitrile (1 mL). The precipitated solids were collected by filtration and washed with MeCN (3×0.2 mL) to provide N-(5-(3-fluoro-1-methyl-1H-pyrrol-2-yl)-1,3,4-thiadiazol-2-yl)-3-(2-methoxyethoxy)-4-(3-methoxypyridin-2-yl)-2-oxo-2H-pyran-6-carboxamide (Compound 18) (5.8 mg, 13% yield). LCMS (ES, m/z)=502.1 [M+H]+; ¹H NMR (400 MHz, DMSO-d6) δ 8.31 (d, J=4.8 Hz, 1H), 7.65 (dd, J=8.4, 1.2 Hz, 1H), 7.59-7.43 (m, 2H), 7.01 (d, J=5.6 Hz, 1H), 6.15 (d, J=2.4 Hz, 1H), 4.39-4.17 (m, 2H), 3.95 (s, 3H), 3.87 (s, 3H), 3.51-3.41 (m, 2H), 3.10 (s, 3H).

### Example 15: N-(5-(5-amino-1H-pyrazol-1-yl)-1,3,4-thiadiazol-2-yl)-4-(2,4-dimethoxypyridin-3-yl)-3-(2-methoxyethoxy)-2-oxo-2H-pyran-6-carboxamide (Compound 19) and N-(5-(5-acetamido-1H-pyrazol-1-yl)-1,3,4-thiadiazol-2-yl)-4-(2,4-dimethoxypyridin-3-yl)-3-(2-methoxyethoxy)-2-oxo-2H-pyran-6-carboxamide (Compound 19-Ac)

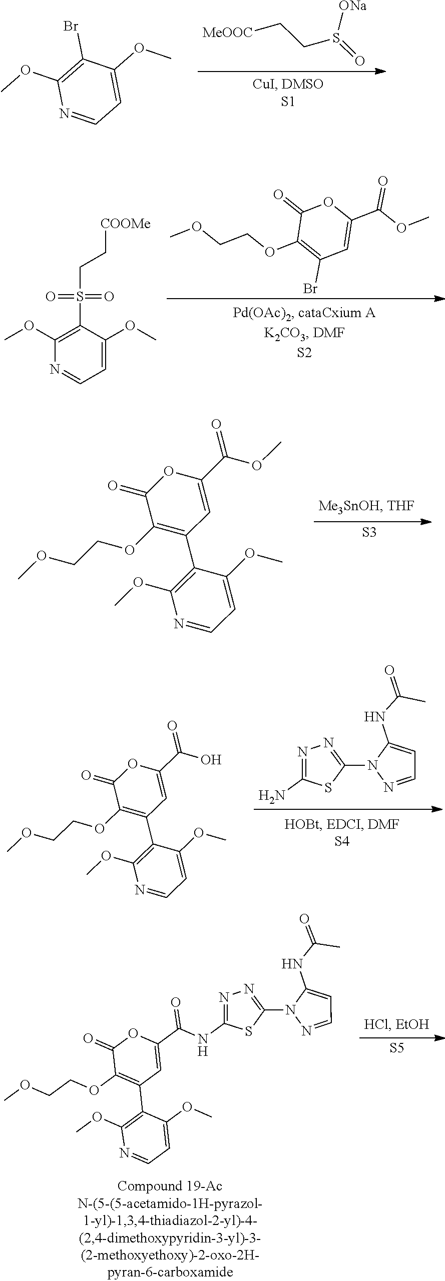

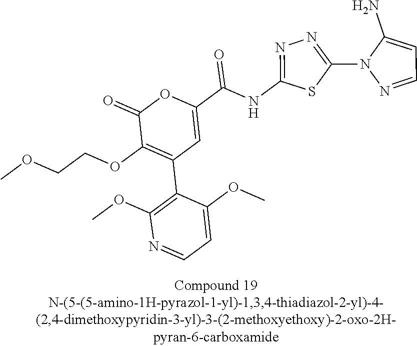 [SMILES: COCCOc1c(-c2c(OC)ccnc2OC)cc(C(=O)Nc2nnc(-n3nccc3N)s2)oc1=O]

Steps 1-3: 4-(2,4-dinethoxypyridin-3-yl)-3-(2-methoxyethoxy)-2-oxo-2H-pyran-6-carboxylic acid was prepared according to Steps 1-3 of Example 6 using 3-bromo-2,4-dimethoxypyridine in place of 2-bromopyridine-3-carbonitrile. LCMS (ESI, m/z)=352.2 [M+H]⁺.

Step 4: N-(5-(5-acetamido-1H-pyrazol-1-yl)-1,3,4-thiadiazol-2-yl)-4-(2,4-dimethoxypyridin-3-yl)-3-(2-methoxyethoxy)-2-oxo-2H-pyran-6-carboxamide (Compound 19-Ac) was prepared according to Step 1 of Example 8 using 4-(2,4-dinethoxypyridin-3-yl)-3-(2-methoxyethoxy)-2-oxo-2H-pyran-6-carboxylic acid (“aryl-pyrone reagent”) in place of 4-(3-cyanopyridin-2-yl)-5-(2-methoxyethoxy)-6-oxopyran-2-carboxylic acid and using N—(I-(5-amino-1,3,4-thiadiazol-2-yl)-1H-pyrazol-5-yl)acetamide (product of Step 3, Example 5; “ADT amine reagent”). LCMS (ESI, m/z)=558.2 [M+H]⁺.

Step 5: N-(5-(5-amino-1H-pyrazol-1-yl)-1,3,4-thiadiazol-2-yl)-4-(2,4-dimethoxypyridin-3-yl)-3-(2-methoxyethoxy)-2-oxo-2H-pyran-6-carboxamide (Compound 19) was prepared according to Step 2 of Example 8 from acidic deprotection of N-(5-(5-acetamido-1H-pyrazol-1-yl)-1,3,4-thiadiazol-2-yl)-4-(2,4-dimethoxypyridin-3-yl)-3-(2-methoxyethoxy)-2-oxo-2H-pyran-6-carboxamide (Compound 19-Ac). LCMS (ES, m/z)=516.1 [M+H]+; ¹H NMR (40 MHz, DMSO-d6) δ 8.17 (d, J=6.0 Hz, 1H), 7.33 (d, J=2.0 Hz, 1H), 6.92-6.90 (m, 2H), 6.60 (s, 2H), 5.37 (d, J=2.0 Hz, 1H), 4.16-4.13 (m, 2H), 3.85 (s, 6H), 3.38 (t, J=4.8 Hz, 2H), 3.09 (s, 3H).

### Example 16: N-(5-(5-amino-1H-pyrazol-1-yl)-1,3,4-thiadiazol-2-yl)-4-(2,6-dimethoxyphenyl)-3-((1,3-dimethoxypropan-2-yl)oxy)-2-oxo-2H-pyran-6-carboxamide (Compound 20) and N-(5-(5-acetamido-1H-pyrazol-1-yl)-1,3,4-thiadiazol-2-yl)-4-(2,6-dimethoxyphenyl)-3-((1,3-dimethoxypropan-2-yl)oxy)-2-oxo-2H-pyran-6-carboxamide (Compound 20-Ac)

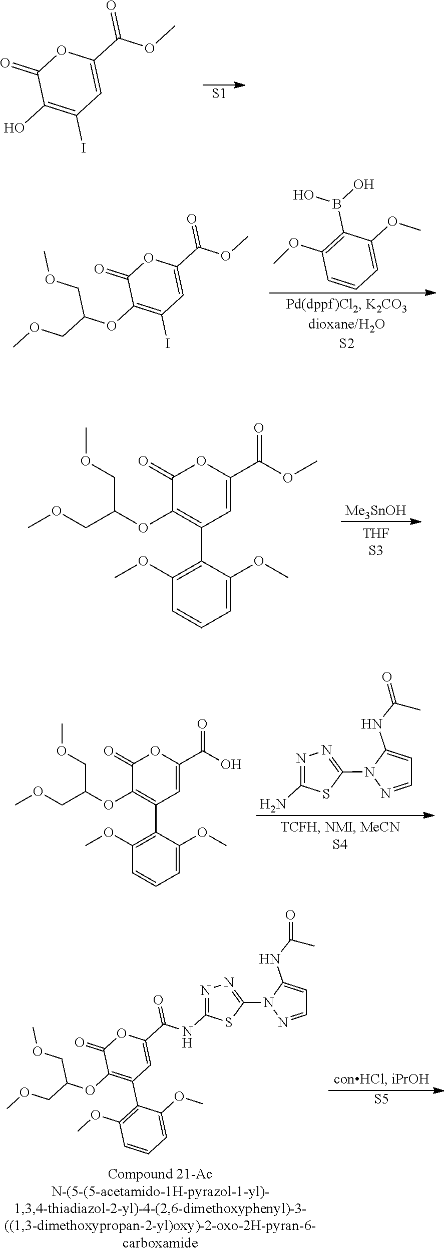

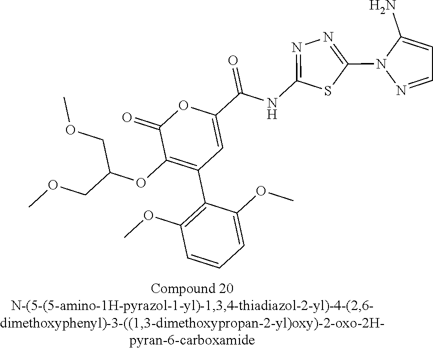 [SMILES: COCC(COC)Oc1c(-c2c(OC)cccc2OC)cc(C(=O)Nc2nnc(-n3nccc3N)s2)oc1=O]

Step 1: To a stirred solution of methyl 5-hydroxy-4-iodo-6-oxopyran-2-carboxylate (product of Step 1, Example 11) (500 mg, 1.69 mmol, 1 equiv) and 1,3-dimethoxypropan-2-ol (304 mg, 2.53 mmol, 1.50 equiv) in tetrahydrofuran (THF) (5 mL) was added triphenyl phosphine (PPh₃) (665 mg, 2.53 mmol, 1.5 equiv) and diethyl azodicarboxylate (DEAD) (441 mg, 2.53 mmol, 1.5 equiv) at 0° C. under nitrogen atmosphere. The resulting mixture was stirred for 1 h at room temperature under nitrogen atmosphere then water (50 mL) was added and the mixture extracted with ethyl acetate (EtOAc) (3×100 mL). The combined organic layers were washed with brine, dried over anhydrous Na₂SO₄, filtered, and concentrated under reduced pressure. The resulting residue was purified by C18 reverse phase flash chromatography (mobile phase, MeCN in water (10 mmol/L NH₄HCO₃), 5% to 95% gradient in 30 min; detector, UV 254 nm) to provide methyl 5-[(1,3-dimethoxypropan-2-yl)oxy]-4-iodo-6-oxopyran-2-carboxylate (550 mg, 82% yield). LCMS (ES, m/z)=398.9 [M+H]+.

Step 2: A solution of methyl 5-[(1,3-dimethoxypropan-2-yl)oxy]-4-iodo-6-oxopyran-2-carboxylate (300 mg, 0.753 mmol, 1 equiv), 2,6-dimethoxyphenylboronic acid (274 mg, 1.51 mmol, 2 equiv), [1,1′-bis(diphenylphosphino)ferrocene]dichloro palladium(II) (Pd(dppf)Cl₂) (110 mg, 0.151 mmol, 0.2 equiv) and K₂CO₃ (3.12 g, 2.26 mmol, 3 equiv) in dioxane (3 mL) and H₂O (0.3 mL) was stirred for 2 h at 70° C. under nitrogen atmosphere. The mixture was then cooled to rt and concentrated under vacuum. The resulting residue was purified by C18 reverse phase flash chromatography (mobile phase, MeCN in water (10 mmol/L NH₄HCO₃), 0% to 50% gradient in 20 min; detector, UV 254 nm) to provide methyl 4-(2,6-dimethoxyphenyl)-5-[(1,3-dimethoxypropan-2-yl)oxy]-6-oxopyran-2-carboxylate (150 mg, 49% yield). LCMS (ES, m/z)=409.1 [M+H]⁺.

Step 3: To a solution of methyl 4-(2,6-dimethoxyphenyl)-5-[(1,3-dimethoxypropan-2-yl)oxy]-6-oxopyran-2-carboxylate (110 mg, 0.269 mmol, 1equiv) in tetrahydrofuran (THF) (2 mL) was added trimethylstannanol (97 mg, 0.54 mmol, 2 equiv). The mixture was stirred for 1 h at room temperature under nitrogen atmosphere then concentrated under vacuum. The resulting residue was purified by C18 reverse phase flash chromatography (mobile phase, MeCN in water (0.1% FA), 0% to 60% gradient in 20 min, detector. UV 254 nm) to provide 4-(2,6-dimethoxyphenyl)-5-[(1,3-dimethoxypropan-2-yl)oxy]-6-oxopyran-2-carboxylic acid (60 mg, 56% yield). LCMS (ES, m/z)=395.1 [M+H]+.

Step 4: N-(5-(5-acetamido-1H-pyrazol-1-yl)-1,3,4-thiadiazol-2-yl)-4-(2,6-dimethoxyphenyl)-3-((1,3-dimethoxypropan-2-yl)oxy)-2-oxo-2H-pyran-6-carboxamide (Compound 20-Ac) was prepared according to Step 1 of Example 8 using 4-(2,6-dimethoxyphenyl)-5-[(1,3-dimethoxypropan-2-yl)oxy]-6-oxopyran-2-carboxylic acid (“aryl-pyrone reagent”) in place of 4-(3-cyanopyridin-2-yl)-5-(2-methoxyethoxy)-6-oxopyran-2-carboxylic acid and coupling with N-(1-(5-amino-1,3,4-thiadiazol-2-yl)-1H-pyrazol-5-yl)acetamide (product of Step 3, Example 5; “ADT amine reagent”). LCMS (ES, m/z)=601.1 [M+H]+.

Step 5: N-(5-(5-amino-1H-pyrazol-1-yl)-1,3,4-thiadiazol-2-yl)-4-(2,6-dimethoxyphenyl)-3-((1,3-dimethoxypropan-2-yl)oxy)-2-oxo-2H-pyran-6-carboxamide (Compound 20) was prepared according to Step 2 of Example 7 by acidic deprotection of N-(5-(5-acetamido-1H-pyrazol-1-yl)-1,3,4-thiadiazol-2-yl)-4-(2,6-dimethoxyphenyl)-3-((1,3-dimethoxypropan-2-yl)oxy)-2-oxo-2H-pyran-6-carboxamide (Compound 20-Ac), using isopropanol (iPrOH) in place of ethanol (EtOH) and 60° C. instead of rt. LCMS (ES, m/z)=559.1 [M+H]+; ¹H NMR (300 MHz, DMSO-d6) δ 7.49-7.39 (m, 2H), 7.27 (s, 1H), 6.80 (s, 1H), 6.77 (s, 1H), 5.52 (d, J=2.0 Hz, 1H), 4.61-4.57 (m, 1H), 3.76 (s, 6H), 3.27-3.24 (m, 4H), 3.07 (s, 6H).

### Example 17: N-(5-(5-amino-1H-pyrazol-1-yl)-1,3,4-thiadiazol-2-yl)-3-(3-methoxypropoxy)-4-(3-methoxypyridin-2-yl)-2-oxo-2H-pyran-6-carboxamide (Compound 21) and N-(5-(5-acetamido-1H-pyrazol-1-yl)-1,3,4-thiadiazol-2-yl)-3-(3-methoxypropoxy)-4-(3-methoxypyridin-2-yl)-2-oxo-2H-pyran-6-carboxamide (Compound 21-Ac)

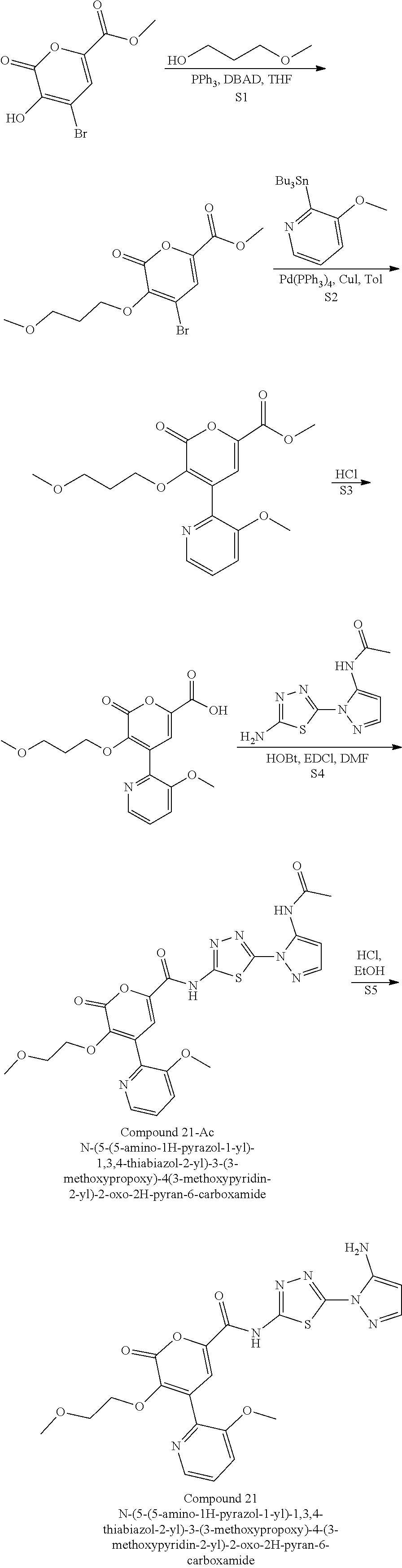

Step 1: To a stirred solution of methyl 4-bromo-5-hydroxy-6-oxopyran-2-carboxylate (product of Step 2, Example 1) (1000 mg, 4.016 mmol, 1 equiv), 3-methoxypropan-1-ol (434 mg, 4.82 mmol, 1.2 equiv) and triphenyl phosphine (PPh₃) (1578 mg, 6.016 mmol, 1.5 equiv) in tetahydrofuran (THF) (15 mL) was added di-tert-butyl azodicarboxylate (DBAD) (1386 mg, 6.019 mmol, 1.5 equiv) in portions at 0° C. under nitrogen atmosphere. The resulting mixture was stirred overnight at room temperature under nitrogen atmosphere. The mixture was then diluted with ethyl acetate (EtOAc) (90 mL), washed with water (3×10 mL), then brine (10 mL), dried over anhydrous Na₂SO₄, filtered, and concentrated under reduced pressure. The resulting residue was purified by silica gel column chromatography (eluting with petroleum ether/ethyl acetate (PE/EtOAc) (12:1)) to afford methyl 4-bromo-5-(3-methoxypropoxy)-6-oxopyran-2-carboxylate (1200 mg, 93% yield). LCMS (ES, m/z)=321.0 [M+H]+.

Steps 2-4: N-(5-(5-acetamido-1H-pyrazol-1-yl)-1,3,4-thiadiazol-2-yl)-3-(3-methoxypropoxy)-4-(3-methoxypyridin-2-yl)-2-oxo-2H-pyran-6-carboxamide (Compound 21-Ac) was prepared according to Steps 1-3 of Example 9 using methyl 4-bromo-5-(3-methoxypropoxy)-6-oxopyran-2-carboxylate in place of methyl 4-bromo-3-(2-methoxyethoxy)-2-oxo-2H-pyran-6-carboxylate. LCMS (ES, m/z)=542.0 [M+H]+.

Step 5: N-(5-(5-amino-1H-pyrazol-1-yl)-1,3,4-thiadiazol-2-yl)-3-(3-methoxypropoxy)-4-(3-methoxypyridin-2-yl)-2-oxo-2H-pyran-6-carboxamide (Compound 21) was prepared according to Step 4 of Example 9 by acidic deprotection of N-(5-(5-acetamido-1H-pyrazol-1-yl)-1,3,4-thiadiazol-2-yl)-3-(3-methoxypropoxy)-4-(3-methoxypyridin-2-yl)-2-oxo-2H-pyran-6-carboxamide (Compound 21-Ac). LCMS (ES, m/z)=500.1 [M+H]+; ¹H NMR (400 MHz, DMSO-d₆ ) δ 13.30 (s, 1H), 8.31 (d, J=3.6 Hz, 1H), 7.67 (dd, J=8.8, 1.2 Hz, 1H), 7.54-7.48 (m, 3H), 6.65 (s, 2H), 5.45 (d, J=1.6 Hz, 1H), 4.17 (t, J=6.4 Hz, 2H), 3.87 (s, 3H), 3.16 (t, J=6.4 Hz, 2H), 3.11 (s, 3H), 1.74-1.68 (m, 2H).

### Example 18: N-(5-(5-amino-1H-pyrazol-1-yl)-1,3,4-thiadiazol-2-yl)-4-(2,6-dimethoxyphenyl)-3-(2-morpholinoethoxy)-2-oxo-2H-pyran-6-carboxamide (Compound 22) and N-(5-(5-acetamido-1H-pyrazol-1-yl)-1,3,4-thiadiazol-2-yl)-4-(2,6-dimethoxyphenyl)-3-(2-morpholinoethoxy)-2-oxo-2H-pyran-6-carboxamide (Compound 22-Ac)

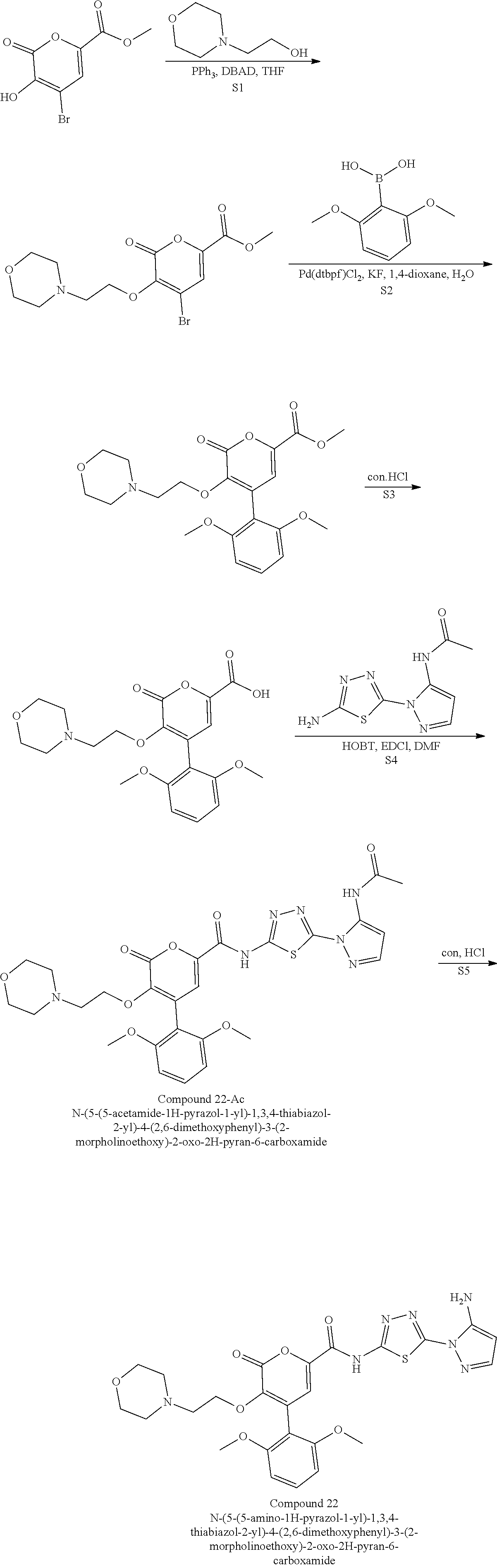

Step 1: To a solution of methyl 4-bromo-5-hydroxy-6-oxopyran-2-carboxylate (product of Step 2, Example 1) (500 mg, 2.01 mmol, 1 equiv) in tetrahydrofuran (THF) (12.5 mL) was added triphenyl phosphine (PPh₃) (790 mg, 3.01 mmol, 1.5 equiv) at room temperature. To the above mixture was added di-tert-butyl azodicarboxylate (DBAD) (693 mg, 3.01 mmol, 1.5 equiv) in portions for 10 min at 0° C. To the above mixture was then added 4-morpholineethanol (316 mg, 2.41 mmol, 1.2 equiv) at room temperature. The mixture was stirred for 1 h at room temperature then diluted with water (50 mL). The solution was extracted with ethyl acetate (EtOAc) (3×20 mL), dried over anhydrous Na₂SO₄, filtered, concentrated under reduced pressure, and the residue was purified by silica gel column chromatography (eluting with petroleum ether/ethyl acetate (PE/EtOAc) (1:1)) to afford methyl 4-bromo-5-[2-(morpholin-4-yl)ethoxy]-6-oxopyran-2-carboxylate (530 mg, 59% yield). LCMS (ES, m/z)=362.0 [M+H]+.

Step 2: To a solution of methyl 4-bromo-5-[2-(morpholin-4-yl)ethoxy]-6-oxopyran-2-carboxylate (345 mg, 0.95 mmol, 1 equiv) in 1,4-dioxane (15.0 mL) and H₂O (3.0 mL) was added 2,6-dimethoxyphenylboronic acid (258 mg, 1.42 mmol, 1.49 equiv), [1,1′-bis(di-tert-butylphosphino)ferrocene]dichloropalladium(II) (Pd(dtbpf)Cl₂) (124 mg, 0.19 mmol, 0.2 equiv) and KF (166 mg, 2.85 mmol, 3 equiv) at room temperature. The resulting mixture was stirred for 2 h at 80° C. under nitrogen atmosphere then cooled to rt and diluted with water (50 mL). The solution was extracted with ethyl acetate (EtOAc) (3×50 mL), dried over anhydrous Na₂SO₄, filtered, and concentrated under reduced pressure. The resulting residue was purified by silica gel column chromatography (eluting with petroleum ether/ethyl acetate (PE/EtOAc) (5:1)) to afford methyl 4-(2,6-dimethoxyphenyl)-5-[2-(morpholin-4-yl)ethoxy]-6-oxopyran-2-carboxylate (270 mg, 68% yield). LCMS (ES, m/z)=420.1 [M+H]+.

Step 3: A solution of methyl 4-(2,6-dimethoxyphenyl)-5-[2-(morpholin-4-yl)ethoxy]-6-oxopyran-2-carboxylate (243 mg, 0.58 mmol, 1 equiv) in conc. HCl (12 mL) was stirred for 1 h at 80° C. The mixture was then cooled to rt and concentrated under reduced pressure to provide 4-(2,6-dimethoxyphenyl)-5-[2-(morpholin-4-yl)ethoxy]-6-oxopyran-2-carboxylic acid (307 mg, 76% yield) which was used directly in the next step without further purification. LCMS (ES, m/z)=406.1 [M+H]+.

Step 4: N-(5-(5-acetamido-1H-pyrazol-1-yl)-1,3,4-thiadiazol-2-yl)-4-(2,6-dimethoxyphenyl)-3-(2-morpholinoethoxy)-2-oxo-2H-pyran-6-carboxamide (Compound 22-Ac) was prepared according to was prepared according to Step 1 of Example 8 using 4-(2,6-dimethoxyphenyl)-5-[2-(morpholin-4-yl)ethoxy]-6-oxopyran-2-carboxylic acid as the “aryl-pyrone reagent” in place of 4-(3-cyanopyridin-2-yl)-5-(2-methoxyethoxy)-6-oxopyran-2-carboxylic acid and using N-(1-(5-amino-1,3,4-thiadiazol-2-yl)-1H-pyrazol-5-yl)acetamide as the “ADT amine reagent”.

Step 5: N-(5-(5-amino-1H-pyrazol-1-yl)-1,3,4-thiadiazol-2-yl)-4-(2,6-dimethoxyphenyl)-3-(2-morpholinoethoxy)-2-oxo-2H-pyran-6-carboxamide (Compound 22) was prepared according to Step 2 of Example 8 by acidic deprotection of N-(5-(5-acetamido-1H-pyrazol-1-yl)-1,3,4-thiadiazol-2-yl)-4-(2,6-dimethoxyphenyl)-3-(2-morpholinoethoxy)-2-oxo-2H-pyran-6-carboxamide (Compound 22-Ac). LCMS (ES, m/z)=570.1 [M+H]+; ¹H NMR (400 MHz, DMSO-d6) δ 7.48-7.46 (m, 2H), 7.34 (s, 1H), 6.83-6.81 (m, 2H), 6.71 (br, 1H), 5.45 (d, J=1.6 Hz, 1H), 4.38-4.36 (m, 2H) 3.78 (s, 6H), 3.52-3.39 (m, 4H), 3.36-3.47 (m, 6H).

### Example 19: 4-(2-cyano-6-methoxyphenyl)-N-(5-(5-(difluoromethyl)-1H-pyrazol-1-yl)-1,3,4-thiadiazol-2-yl)-3-(2-methoxyethoxy)-2-oxo-2H-pyran-6-carboxamide (Compound 23)

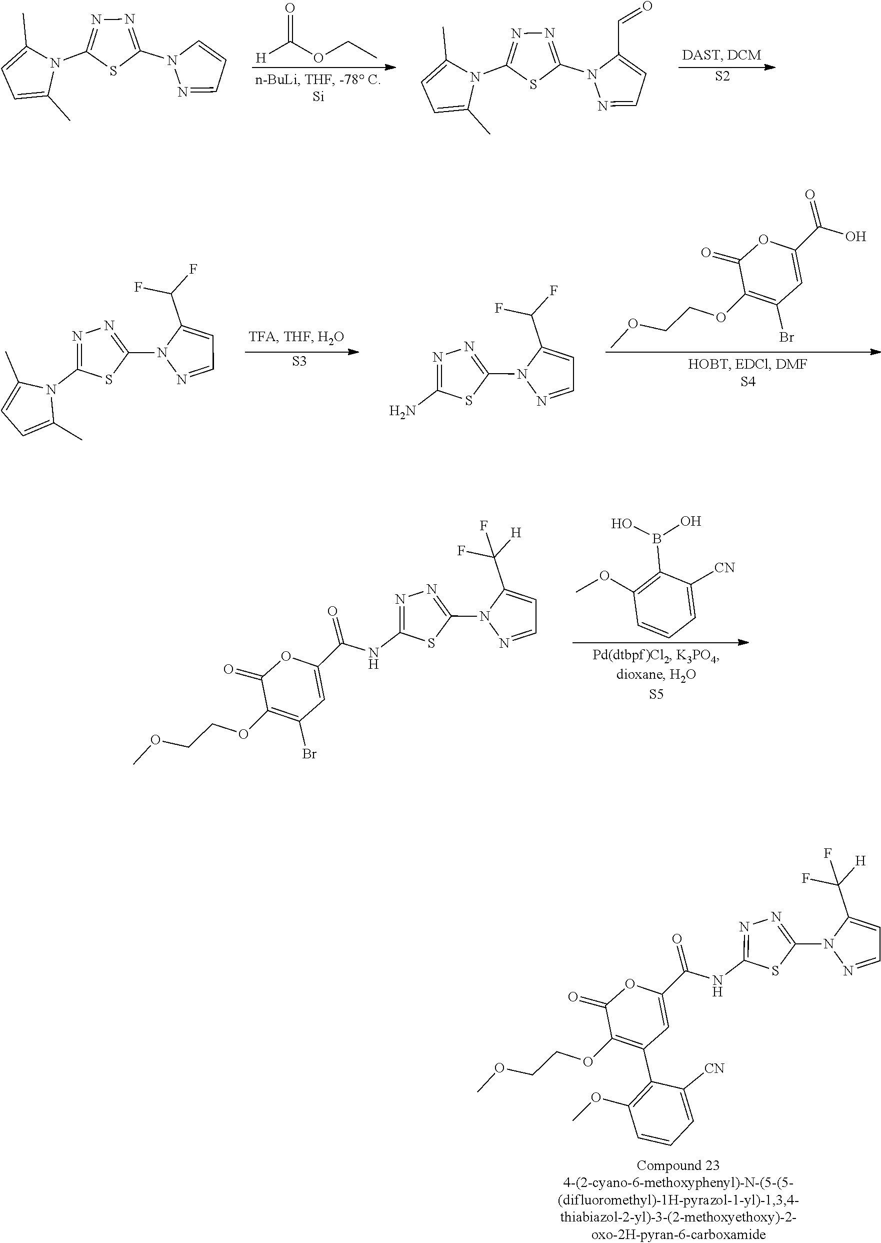

Step 1: Into a solution of 2-(2,5-dimethylpyrrol-1-yl)-5-(pyrazol-1-yl)-1,3,4-thiadiazole (5 g, 20 mmol, 1 equiv) in tetrahydrofuran (THF) (100 mL) was added n-butyl lithium (n-BuLi) (8.97 mL, 22.4 mmol, 1.1 equiv) dropwise over 5 min at −78° C. The resulting mixture was stirred for 1 h at −78° C. To the mixture was then added ethyl formate (4.53 g, 61.1 mmol, 3equiv) dropwise over 5 min at −78° C. The mixture was stirred for an additional 1 h at −78° C. then quenched with sat. NH₄Cl (aq.) at 0° C. The aqueous layer was extracted with ethyl acetate (EtOAc) (3×100 mL). The resulting mixture was concentrated under reduced pressure and the resulting residue was purified by silica gel column chromatography, eluting with petroleum ether/ethyl acetate (PE/EtOAc) (8:1), to afford 2-[5-(2,5-dimethylpyrrol-1-yl)-1,3,4-thiadiazol-2-yl]pyrazole-3-carbaldehyde (3.5 g, 56% yield). LCMS (ESI, m/z)=274.2 [M+1]+.

Step 2: To a stirred solution of 2-[5-(2,5-dimethylpyrrol-1-yl)-1,3,4-thiadiazol-2-yl]pyrazole-3-carbaldehyde (700 mg, 2.56 mmol, 1 equiv) in dichloromethane (DCM) (5 mL) was added diethylaminosulfur trifluoride (DAST) (826 mg, 5.12 mmol, 2equiv) at 0° C. The resulting mixture was stirred for 2 h at room temperature under air atmosphere. The reaction was then quenched by the addition of Water (2 mL) at room temperature and extracted with ethyl acetate (EtOAc) (3×50 mL). The combined organic layers were concentrated under reduced pressure and the residue purified by silica gel column chromatography, eluting with petroleum ether/ethyl acetate (PE/EtOAc) (5:1), to afford 2-[5-(difluoromethyl)pyrazol-1-yl]-5-(2,5-dimethylpyrrol-1-yl)-1,3,4-thiadiazole (300 mg, 40% yield).

Step 3: Into a solution of 2-[5-(difluoromethyl)pyrazol-1-yl]-5-(2,5-dimethylpyrrol-1-yl)-1,3,4-thiadiazole (300 mg, 1.02 mmol, 1 equiv) in tetrahydrofuran (THF) (1 mL) and H₂O (0.5 mL) was added trifluoroacetic acid (TFA) (0.5 mL) at 0° C. The resulting mixture was stirred for 2 h at 60° C. under air atmosphere. The mixture was then concentrated under reduced pressure and the residue purified by silica gel column chromatography, eluting with petroleum ether/ethyl acetate (PE/EtOAc) (5:1), to afford 5-[5-(difluoromethyl)pyrazol-1-yl]-1,3,4-thiadiazol-2-amine (190 mg, 86% yield). LCMS (ESI, m/z)=218.0 [M+1]+.

Step 4: 4-bromo-N-(5-(5-(difluoromethyl)-1H-pyrazol-1-yl)-1,3,4-thiadiazol-2-yl)-3-(2-methoxyethoxy)-2-oxo-2H-pyran-6-carboxamide prepared according to Step 3 of Example 3 using 5-[5-(difluoromethyl)pyrazol-1-yl]-1,3,4-thiadiazol-2-amine in place of 5-(5-methylpyrazol-1-yl)-1,3,4-thiadiazol-2-amine. LCMS (ES, m/z)=491.9 [M+H]+.

Step 5: 4-(2-cyano-6-methoxyphenyl)-N-(5-(5-(difluoromethyl)-1H-pyrazol-1-yl)-1,3,4-thiadiazol-2-yl)-3-(2-methoxyethoxy)-2-oxo-2H-pyran-6-carboxamide (Compound 23) prepared according to Step 1 of Example 7 using 4-bromo-N-(5-(5-(difluoromethyl)-1H-pyrazol-1-yl)-1,3,4-thiadiazol-2-yl)-3-(2-methoxyethoxy)-2-oxo-2H-pyran-6-carboxamide as the “halo-pyrone reagent”
in place of 4-bromo-N-[5-(5-acetamidopyrazol-1-yl)-1,3,4-thiadiazol-2-yl]-5-(2-methoxyethoxy)-6-oxopyran-2-carboxamide and 2-cyano-6-methoxyphenylboronic acid as the “aryl boron reagent” in place of 3-methoxy-2-(4,4,5,5-tetramethyl-1,3,2-dioxaborolan-2-yl)benzonitrile. LCMS (ES, m/z)=545.2 [M+H]+; ¹H NMR (400 MHz, DMSO-d₆) δ 8.03 (d, J=2.0 Hz, 1H), 7.68-7.58 (m, 2H), 7.57-7.52 (m, 2H), 7.39 (s, 1H), 7.06 (d, J=13.6 Hz, 1H), 4.33-4.26 (m, 2H), 3.87 (s, 3H), 3.37-3.25 (m, 2H), 3.05 (s, 3H).

### Example 20: (Ra)—N-(5-(5-amino-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24A*), (Sa)-N-(5-(5-amino-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24B*), and N-(5-(5-acetamido-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24-Ac)

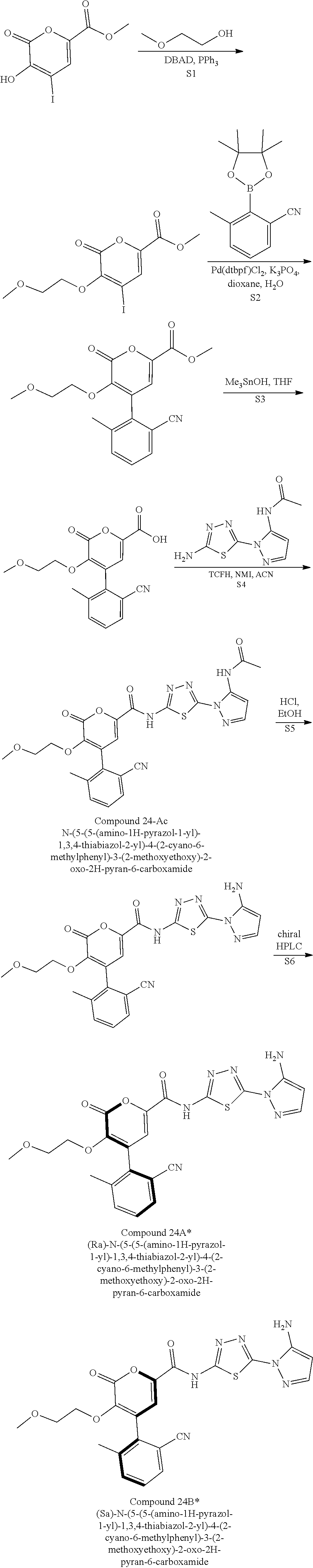

Step 1: Methyl 4-iodo-3-(2-methoxyethoxy)-2-oxo-2H-pyran-6-carboxylate was prepared according to Steps 1-2 of Example 1 and Step 1 of Example 3 using methyl 3-hydroxy-4-iodo-2-oxo-2H-pyran-6-carboxylate in place of methyl 3-hydroxy-4-bromo-2-oxo-2H-pyran-6-carboxylate. LCMS (ES, m/z)=354.8 [M+H]+.

Step 2: Methyl 4-(2-cyano-6-methylphenyl)-3-(2-methoxyethoxy)-2-oxo-2H-pyran-6-carboxylate was prepared according to Step 2 of Example 18 using methyl 4-iodo-3-(2-methoxyethoxy)-2-oxo-2H-pyran-6-carboxylate in place of methyl 4-bromo-5-[2-(morpholin-4-yl)ethoxy]-6-oxopyran-2-carboxylate and 3-methyl-2-(4,4,5,5-tetramethyl-1,3,2-dioxaborolan-2-yl)benzonitrile in place of 2,6-dimethoxyphenylboronic acid. ¹H NMR (400 MHz, DMSO-d6) δ 7.76-7.60 (m, 2H), 7.58-7.45 (m, 1H), 1.36 (s, 3H), 1.17 (s, 12H).

Steps 3-4: N-(5-(5-acetamido-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24-Ac) was prepared according to Steps 3-4 of Example 16 using methyl 4-(2-cyano-6-methylphenyl)-3-(2-methoxyethoxy)-2-oxo-2H-pyran-6-carboxylate in place of methyl 4-(2,6-dimethoxyphenyl)-5-[(1,3-dimethoxypropan-2-yl)oxy]-6-oxopyran-2-carboxylate to provide 4-(2-cyano-6-methylphenyl)-3-(2-methoxyethoxy)-2-oxo-2H-pyran-6-carboxylic acid as the “aryl-pyrone reagent” and using 4-(3-cyanopyridin-2-yl)-5-(2-methoxyethoxy)-6-oxopyran-2-carboxylic acid and using N-(1-(5-amino-1,3,4-thiadiazol-2-yl)-1H-pyrazol-5-yl)acetamide (product of Step 3, Example 5) as the “ADT amine reagent”.

Steps 5-6: N-(5-(5-acetamido-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24-Ac) was deprotected under acidic conditions following Step 5 of Example 16 to provide N-(5-(5-amino-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24), which was separated into constituent axially chiral atropisomers by chiral prep-HPLC (CHIRALPAK IF, 2*25 cm; Mobile Phase A: Hexanes (0.1% TFA), Mobile Phase B: MeOH:DCM=1:1; Flow rate: 20 mL/min; Gradient: 80% B to 80% B in 15 min; Wave Length: 220/254 nm) to afford (Ra)—N-(5-(5-amino-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24A*) (29 mg, 3% yield) as the first eluting peak (Chiral RT=6.64 min), and (Sa)-N-(5-(5-amino-1H-pyrazol-1-yl)-1,3,4-thiadiazol-2-yl)-4-(2-cyano-6-methylphenyl)-3-(2-methoxyethoxy)-2-oxo-2H-pyran-6-carboxamide (Compound 24B*) (22 mg, 2.4% yield) as the second eluting peak (Chiral RT=11.2 min). Stereochemistry arbitrarily assigned.

Compound 24A*: LCMS (ES, m/z)=494.1 [M+H]+, ¹H NMR (400 MHz, DMSO-d6) δ 7.84 (d, J=7.2 Hz, 1H), 7.72 (d, J=7.6 Hz, 1H), 7.58 (d, J=7.6 Hz, 1H), 7.51-7.44 (m, 2H), 6.65 (s, 2H), 5.45 (d, J=2.0 Hz, 1H), 4.34-4.30 (m, 2H), 3.35 (d, J=4.4 Hz, 2H), 3.03 (s, 3H), 2.28 (s, 3H).

Compound 24B*: LCMS (ES, m/z)=494.1 [M+H]+. ¹H NMR (400 MHz, DMSO-d6) δ 13.36 (s, 1H), 7.84 (d, J=7.6 Hz, 1H), 7.72 (d, J=7.6 Hz, 1H), 7.58 (t, J=8.0 Hz, 1H), 7.51-7.44 (m, 2H), 6.65 (s, 2H), 5.45 (d, J=1.6 Hz, 1H), 4.34-4.30 (m, 2H), 3.35 (d, J=4.4 Hz, 2H), 3.03 (s, 3H), 2.28 (s, 3H).

### Example 21: N-(5-(5-chloro-1H-pyrazol-1-yl)-1,3,4-thiadiazol-2-yl)-4-(2-cyano-6-methoxyphenyl)-3-(2-hydroxyethoxy)-2-oxo-2H-pyran-6-carboxamide (Compound 26) and 3-(2-(tert-butoxy)ethoxy)-N-(5-(5-chloro-1H-pyrazol-1-yl)-1,3,4-thiadiazol-2-yl)-4-(2-cyano-6-methoxyphenyl)-2-oxo-2H-pyran-6-carboxamide (Compound 26-OtBu)

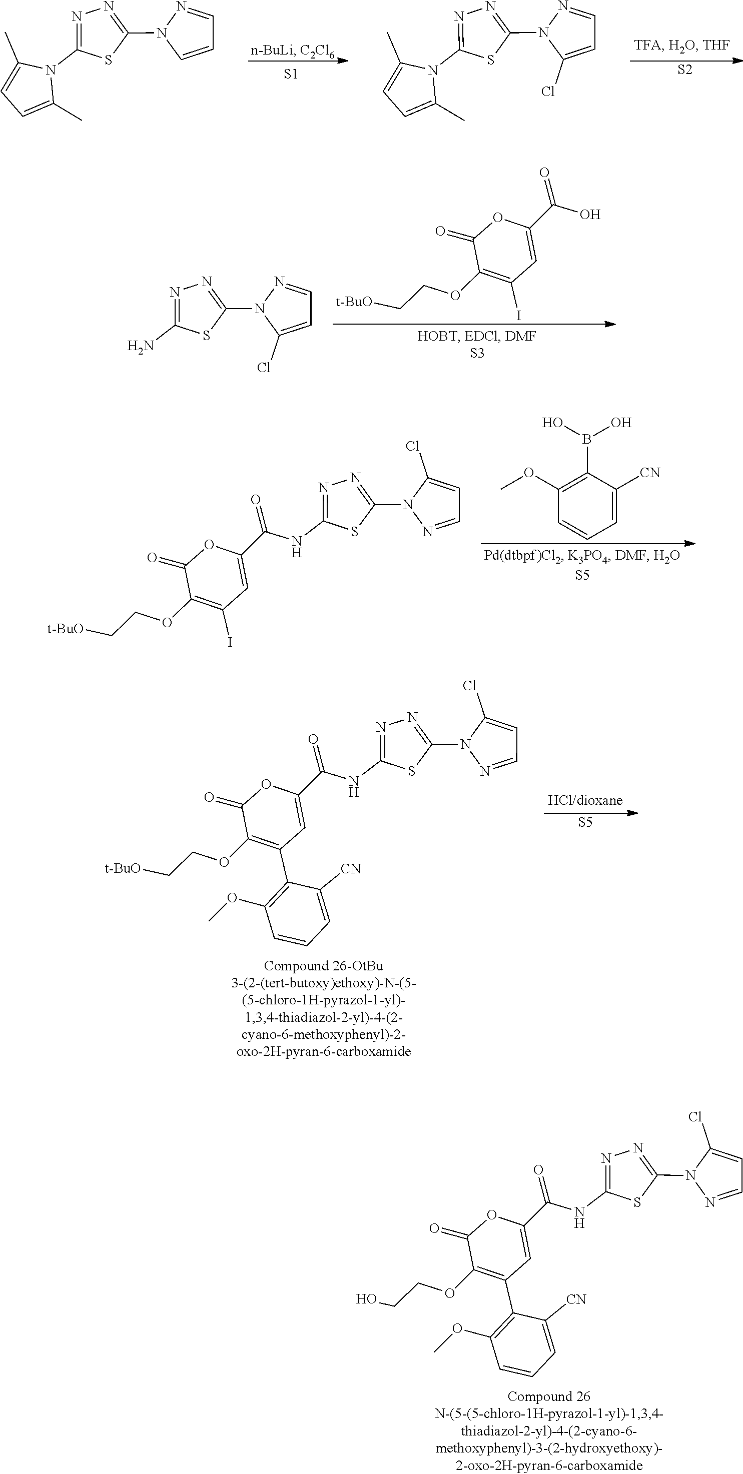

Step 1: A solution of 2-(2,5-dimethylpyrrol-1-yl)-5-(pyrazol-1-yl)-1,3,4-thiadiazole (1000 mg, 4.077 mmol, 1 equiv) in tetrahydrofuran (THF) was added dropwise n-butyl lithium (n-BuLi) (2.4 mL, 6.0 mmol, 1.5 equiv) at −78° C. under N₂ (nitrogen gas). The solution was stirred at −78° C. for 1 h followed by the addition of hexachloroethane (C₂Cl₆) (1000 mg, 4.22 mmol, 1 equiv) dropwise at −78° C. The resulting mixture was stirred for 2 h at room temperature under N₂ (nitrogen gas) then quenched with sat. NH₄Cl (aq.) at 0° C. The aqueous layer was extracted with ethyl acetate (EtOAc) (3×30 mL). The extracts were concentrated under reduced pressure and the resulting residue was purified by silica gel column chromatography, eluting with petroleum ether/ethyl acetate (PE/EtOAc) (9:1), to afford 2-(5-chloropyrazol-1-yl)-5-(2,5-dimethylpyrrol-1-yl)-1,3,4-thiadiazole (880 mg, 76% yield). LCMS (ESI, m/z)=280.0 [M+1]+.

Step 2: Into a solution of 2-(5-chloropyrazol-1-yl)-5-(2,5-dimethylpyrrol-1-yl)-1,3,4-thiadiazole (880 mg, 3.15 mmol, 1 equiv) in tetrahydrofuran (THF) (1 mL) and H₂O (2 mL) was added trifluoroacetic acid (TFA) (2 mL) at room temperature. The resulting mixture was stirred for 3 h at 60° C. then concentrated under reduced pressure. The residue was purified by C18 reverse phase flash chromatography (acetonitrile (MeCN) in water, 10% to 30% gradient in 7 min; detector, UV 254 nm) to afford 5-(5-chloropyrazol-1-yl)-1,3,4-thiadiazol-2-amine (390 mg, 61% yield). LCMS (ESI, m/z)=202.0 [M+1]+.

Step 3: To a solution of 5-(5-chloropyrazol-1-yl)-1,3,4-thiadiazol-2-amine (200 mg, 0.99 mmol, 1 equiv) in N,N-dimethylformamide (DMF) (10 mL) was added hydroxybenzotriazole (HOBT) (380 mg, 2.81 mmol, 2.83 equiv), 1-ethyl-3-(3-dimethylaminopropyl)carbodiimide (EDCI) (148 mg, 0.77 mmol, 0.78 equiv) at room temperature. The resulting mixture was stirred for 5 min at room temperature then 5-[2-(tert-butoxy)ethoxy]-4-iodo-6-oxopyran-2-carboxylic acid (product of Step 3, Example 13) (346 mg, 0.904 mmol, 0.91 equiv) was added at room temperature. The mixture was stirred until completion at rt by LCMS then directly purified by C18 reverse phase flash chromatography (mobile phase, MeCN in water (0.1% TFA), 40% to 50% gradient in 10 min; detector, UV 254 nm) to provide 5-[2-(tert-butoxy)ethoxy]-N-[5-(5-chloropyrazol-1-yl)-1,3,4-thiadiazol-2-yl]-4-iodo-6-oxopyran-2-carboxamide (320 mg, 57% yield). LCMS (ES, m/z)=566.1 [M+H]+.

Step 4: To a solution of 5-[2-(tert-butoxy)ethoxy]-N-[5-(5-chloropyrazol-1-yl)-1,3,4-thiadiazol-2-yl]-4-iodo-6-oxopyran-2-carboxamide (“halo-pyrone reagent”) (280 mg, 0.495 mmol, 1 equiv) in N,N-dimethylformamide (DMF) (7 mL) and H₂O (0.7 mL) was added 2-cyano-6-methoxyphenylboronic acid (“aryl boron reagent”) (392 mg, 2.213 mmol, 4.5 equiv), K₃PO₄ (314 mg, 1.48 mmol, 3 equiv) and [1,1′-bis(di-tert-butylphosphino)ferrocene]dichloropalladium(II) (Pd(dtbpf)Cl₂) (97 mg, 0.15 mmol, 0.3 equiv) at room temperature. The reaction mixture was then irradiated in a microwave reactor for 20 min at 120° C. under a nitrogen atmosphere. The mixture was cooled to rt and diluted with ethyl acetate (EtOAc) (100 mL) and washed with water (3×10 mL). The organic extract was dried over anhydrous Na₂SO₄, filtered, and concentrated under reduced pressure. The resulting residue was purified by silica gel column chromatography (eluting with MeCN/H₂O (7:1)) to afford 3-(2-(tert-butoxy)ethoxy)-N-(5-(5-chloro-1H-pyrazol-1-yl)-1,3,4-thiadiazol-2-yl)-4-(2-cyano-6-methoxyphenyl)-2-oxo-2H-pyran-6-carboxamide (Compound 26-OtBu) (128 mg, 45% yield). LCMS (ES, m/z)=569.1 [M−H]—.

Step 5: To a solution of 4M HCl in 1,4-dioxane (8 mL) was added 3-(2-(tert-butoxy)ethoxy)-N-(5-(5-chloro-1H-pyrazol-1-yl)-1,3,4-thiadiazol-2-yl)-4-(2-cyano-6-methoxyphenyl)-2-oxo-2H-pyran-6-carboxamide (Compound 26-OtBu) (130 mg, 0.228 mmol, 1 equiv). The resulting solution was stirred for 1 h at 80° C. then cooled to rt and concentrated under reduced pressure. The resulting residue was purified by C18 reverse phase flash chromatography (mobile phase, MeCN in water (0.1% TFA), 40% to 50% gradient in 20 min; detector, UV 254 nm) to provide N-(5-(5-chloro-1H-pyrazol-1-yl)-1,3,4-thiadiazol-2-yl)-4-(2-cyano-6-methoxyphenyl)-3-(2-hydroxyethoxy)-2-oxo-2H-pyran-6-carboxamide (Compound 26) (5.6 mg, 4.7% yield). LCMS (ES, m/z)=515.0[M+H]+; ¹H NMR (400 MHz, DMSO-d6) δ 8.02 (s, 1H), 7.69-7.47 (m, 4H), 6.86 (s, 1H), 4.22-4.19 (m, 2H), 3.87 (s, 3H), 3.47-3.45 (m, 2H).

### Example 22: N-(5-(5-amino-1H-pyrazol-1-yl)-1,3,4-thiadiazol-2-yl)-4-(2-methoxy-6-(2-methoxyethoxy)phenyl)-3-(2-methoxyethoxy)-2-oxo-2H-pyran-6-carboxamide (Compound 27) and N-(5-(5-acetamido-1H-pyrazol-1-yl)-1,3,4-thiadiazol-2-yl)-4-(2-methoxy-6-(2-methoxyethoxy)phenyl)-3-(2-methoxyethoxy)-2-oxo-2H-pyran-6-carboxamide (Compound 27-Ac)

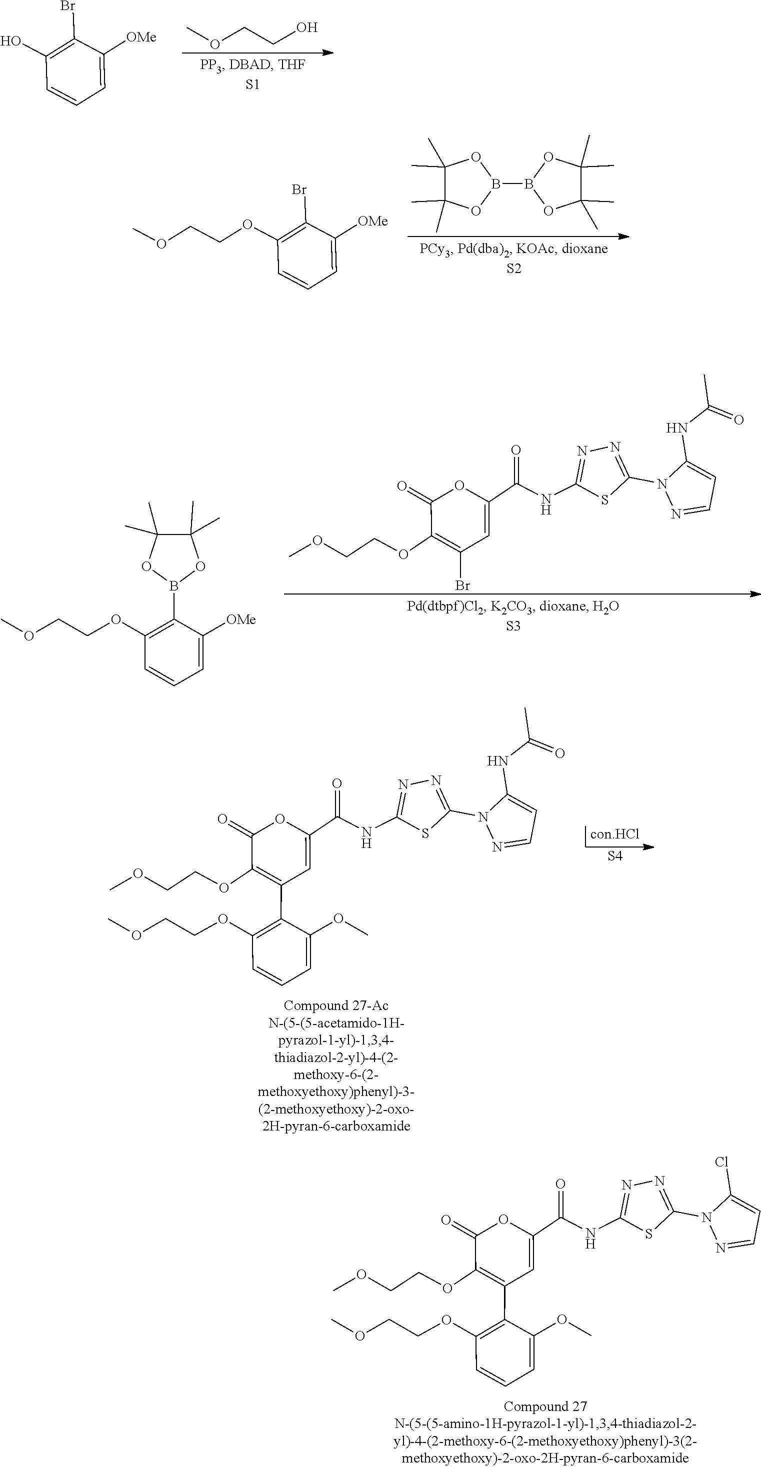

Step 1: To a stirred mixture of 2-bromo-3-methoxyphenol (2 g, 9.85 mmol, 1 equiv) in tetrahydrofuran (THF) (20 mL) was added triphenyl phosphine (PPh₃) (3.87 g, 14.8 mmol, 1.5 equiv) and di-tert-butyl azodicarboxylate (DBAD) (3.40 g, 14.8 mmol, 1.5 equiv) in tetrahydrofuran (THF) (5 ml) at 0° C. The resulting mixture was stirred for 10 min at room temperature then 2-methoxyethanol (899 mg, 11.8 mmol, 1.2 equiv) was added at room temperature. The resulting mixture was stirred overnight at room temperature then quenched by the addition of water (100 mL) at room temperature. The resulting mixture was extracted with ethyl acetate (EtOAc) (3×100 mL) and the combined organic layers were washed with brine (2×100 mL), dried over anhydrous Na₂SO₄, filtered, and concentrated under reduced pressure. The resulting residue was purified by silica gel column chromatography (eluting with petroleum ether/ethyl acetate (PE/EtOAc) (10:1)) to afford 2-bromo-1-methoxy-3-(2-methoxyethoxy)benzene (970 mg, 38% yield). LCMS (ES, m/z)=261.1 [M+H]+.

Step 2: A mixture of his (dibenzylideneacetone)palladium(0) (Pd(dba)₂) (44 mg, 0.077 mmol, 0.10 equiv) and tricyclohexylphospine (PCy₃) (52 mg, 0.18 mmol, 0.2 equiv) in dioxane (2 mL) was stirred for 30 min at room temperature under nitrogen atmosphere. To the above mixture was added a solution of 2-bromo-1-methoxy-3-(2-methoxyethoxy) benzene (200 mg, 0.766 mmol, 1 equiv), potassium acetate (KOAc) (150 mg, 1.528 mmol, 2 equiv) and bis(pinacolato)diboron (290 mg, 1.142 mmol, 1.5 equiv) in dioxane (2 mL) at room temperature. The resulting mixture was stirred for 1 h at 110° C. then cooled to rt and quenched by the addition of water (50 mL). The mixture was extracted with ethyl acetate (EtOAc) (3×50 mL) and the combined organic layers were washed with brine (50 mL), dried over anhydrous Na₂SO₄, filtered, and concentrated under reduced pressure. The resulting residue was purified by silica gel column chromatography (eluting with petroleum ether/ethyl acetate (PE/EtOAc) (10:1)) to afford 2-[2-methoxy-6-(2-methoxyethoxy)phenyl]-4,4,5,5-tetramethyl-1,3,2-dioxaborolane (140 mg, 59% yield). LCMS (ES, m/z)=309.2 [M+H]+.

Step 3: To a stirred mixture of 2-[2-methoxy-6-(2-methoxyethoxy)phenyl]-4,4,5,5-tetramethyl-1,3,2-dioxaborolane (100 mg, 0.324 mmol, 1 equiv) and 4-bromo-N-[5-(5-acetamidopyrazol-1-yl)-1,3,4-thiadiazol-2-yl]-5-(2-methoxyethoxy)-6-oxopyran-2-carboxamide (135 mg, 0.270 mmol, 0.8 equiv) in dioxane (1.2 mL) and H₂O (0.2 mL) was added [1,1′-bis(di-tert-butylphosphino)ferrocene]dichloropalladium(II) (Pd(dtbpf)Cl₂) (35 mg, 0.054 mmol, 0.17 equiv) and K₂CO₃ (75 mg, 0.543 mmol, 1.7 equiv) at room temperature under nitrogen atmosphere. The resulting mixture was stirred for 1 h at 80° C. under nitrogen atmosphere then cooled to rt and diluted with water (50 mL). The mixture was extracted with ethyl acetate (EtOAc) (3×50 mL), and the combined organic layers were washed with brine (50 mL), dried over anhydrous Na₂SO₄, filtered, and concentrated under reduced pressure. The resulting residue was purified by C18 reverse phase flash chromatography (mobile phase. MeCN in water (10 mmol/L NH₄HCO₃), 10% to 50% gradient in 10 min; detector, UV 254 nm) to afford N-(5-(5-acetamido-1H-pyrazol-1-yl)-1,3,4-thiadiazol-2-yl)-4-(2-methoxy-6-(2-methoxyethoxy)phenyl)-3-(2-methoxyethoxy)-2-oxo-2H-pyran-6-carboxamide (Compound 27-Ac) (65 mg, 33% yield). LCMS (ES, m/z)=601.2 [M+H]+.

Step 4: A solution of N-(5-(5-acetamido-1H-pyrazol-1-yl)-1,3,4-thiadiazol-2-yl)-4-(2-methoxy-6-(2-methoxyethoxy)phenyl)-3-(2-methoxyethoxy)-2-oxo-2H-pyran-6-carboxamide (Compound 27-Ac) (28 mg, 0.047 mmol, 1 equiv) in conc. HCl (1 mL) was stirred overnight at room temperature. The mixture was then concentrated under reduced pressure and the resulting residue purified by C18 reverse phase flash chromatography (mobile phase, MeCN in water (0.05% TFA), 40% to 50% gradient in 10 min: detector, UV 254 nm) to provide N-(5-(5-amino-1H-pyrazol-1-yl)-1,3,4-thiadiazol-2-yl)-4-(2-methoxy-6-(2-methoxyethoxy)phenyl)-3-(2-methoxyethoxy)-2-oxo-2H-pyran-6-carboxamide (Compound 27) (8.5 mg, 32% yield). LCMS (ES, m/z)=559.1 [M+H]+; ¹H NMR (400 MHz, DMSO-d6) δ 13.22 (br, 1H), 7.48 (d, J=2.0 Hz, 1H), 7.40 (d, J=8.4 Hz, 1H), 7.32 (s, 1H), 6.83-6.76 (m, 2H), 6.65 (s, 2H), 5.45 (d, J=2.0 Hz, 1H), 4.27-4.07 (m, 4H), 3.78 (s, 3H), 3.60-3.50 (m, 2H), 3.40 (t, J=4.4 Hz, 2H), 3.20 (s, 3H), 3.11 (s, 3H).

### Example 23: N-(5-(5-amino-1H-pyrazol-1-yl)-1,3,4-thiadiazol-2-yl)-4-(3,5-dimethoxypyridin-4-yl)-3-(2-methoxyethoxy)-2-oxo-2H-pyran-6-carboxamide (Compound 28) and N-(5-(5-acetamido-1H-pyrazol-1-yl)-1,3,4-thiadiazol-2-yl)-4-(3,5-dimethoxypyridin-4-yl)-3-(2-methoxyethoxy)-2-oxo-2H-pyran-6-carboxamide (Compound 28-Ac)

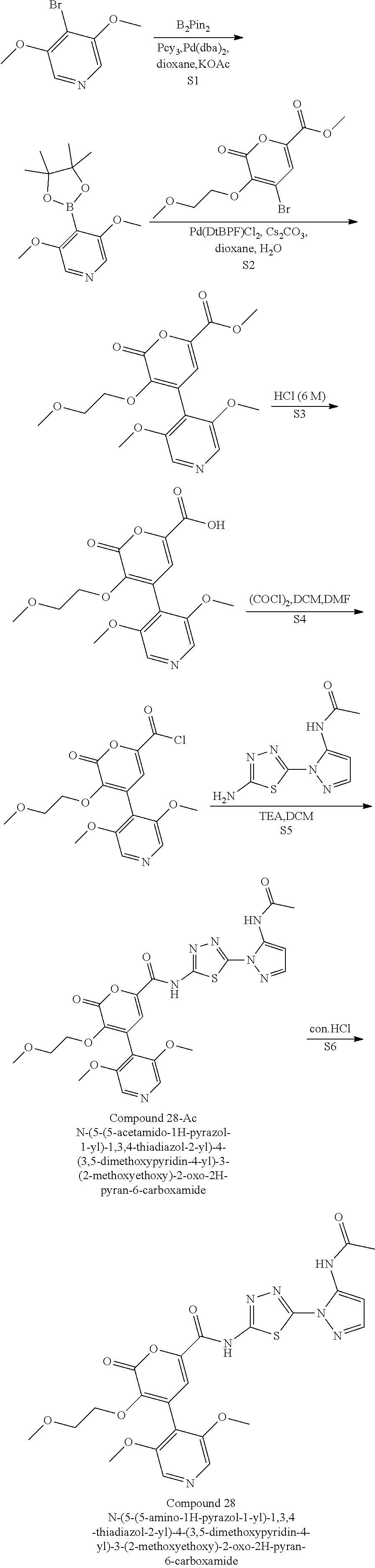

Step 1: Under nitrogen, a solution of his (dibenzylideneacetone)palladium(0) (Pd(dba)₂) (132 mg, 0.23 mmol, 0.1 equiv) and tricyclohexylphospine (PCy₃) (154 mg, 0.55 mmol, 0.2 equiv) in dioxane (15 mL) was stirred for 30 min at room temperature. To the above mixture was then added 4-bromo-3,5-dimethoxypyridine (500 mg, 2.29 mmol, 1 equiv), bis(pinacolato)diboron (873 mg, 3.44 mmol, 1.5 equiv) and potassium acetate (KOAc) (450 mg, 4.58 mmol, 2 equiv) at room temperature. The resulting mixture was stirred for 1 h at 120° C. then cooled to rt and quenched by the addition of water (50 mL). The mixture was extracted with ethyl acetate (EtOAc) (3×100 mL), and the combined organic layers were washed with brine (100 mL), dried over anhydrous Na₂SO₄, filtered, and concentrated under reduced pressure to provide crude 3,5-dimethoxy-4-(4,4,5,5-tetramethyl-1,3,2-dioxaborolan-2-yl)pyridine (500 mg, 82% yield), which was used directly in the next step without further purification. LCMS (ES, m/z)=266.1 [M+H]+.

Step 2: Under nitrogen, to a solution of 3,5-dimethoxy-4-(4,4,5,5-tetramethyl-1,3,2-dioxaborolan-2-yl)pyridine (500 mg, 1.89 mmol, 1 equiv) in 1,4-dioxane (15 mL) and H₂O (2.5 mL) was added methyl 4-bromo-5-(2-methoxyethoxy)-6-oxopyran-2-carboxylate (product of Step 1, Example 3) (579 mg, 1.88 mmol, 1 equiv), [1,1′-bis(di-tert-butylphosphino)ferrocene]dichloropalladium(II) (Pd(dtbpf)Cl₂) (246 mg, 0.38 mmol, 0.2 equiv) and Cs₂CO₃ (1.23 g, 3.78 mmol, 2equiv) at room temperature. The mixture was stirred for 1 h at 80° C. then cooled to rt and quenched by the addition of water (100 mL). The solution was extracted with ethyl acetate (EtOAc) (3×200 mL), and the combined organic layers were washed with brine (100 mL), dried over anhydrous Na₂SO₄, filtered, and concentrated under reduced pressure. The resulting residue was purified by silica gel column chromatography (eluting with CH₂Cl₂/MeOH (9:1)) to afford methyl 4-(3,5-dimethoxypyridin-4-yl)-5-(2-methoxyethoxy)-6-oxopyran-2-carboxylate (130 mg, 17% yield). LCMS (ES, m/z)=365.8 [M+H]+.

Step 3: A solution of methyl 4-(3,5-dimethoxypyridin-4-yl)-5-(2-methoxyethoxy)-6-oxopyran-2-carboxylate (120 mg, 0.33 mmol, 1 equiv) in HCl (2 mL, 6 M) was stirred for 1 h at 80° C. The mixture was then cooled to it and concentrated under reduced pressure to provide 4-(3,5-dimethoxypyridin-4-yl)-5-(2-methoxyethoxy)-6-oxopyran-2-carboxylic acid HCl salt (140 mg, 99% yield) which was used directly in the next step without further purification. LCMS (ES, m/z)=352.1 [M+H]+.

Steps 4-5: To a solution of 4-(3,5-dimethoxypyridin-4-yl)-5-(2-methoxyethoxy)-6-oxopyran-2-carboxylic acid HCl salt (60 mg, 0.17 mmol, 1 equiv) in dichloromethane (DCM) (2.5 mL) was added (COCl)₂ (87 mg, 0.68 mmol, 4 equiv) and N,N-dimethylformamide (DMF) (one drop) at 0° C. The resulting mixture was stirred for 1 h at room temperature then concentrated under reduced pressure to provide crude 4-(3,5-dimethoxypyridin-4-yl)-3-(2-methoxyethoxy)-2-oxo-2H-pyran-6-carbonyl chloride. To a solution of 4-(3,5-dimethoxypyridin-4-yl)-5-(2-methoxyethoxy)-6-oxopyran-2-carbonyl chloride (“aryl-pyrone reagent”) (60 mg, 0.16 mmol, 1 equiv) in DCM (2.5 mL) was added N-(1-(5-amino-1,3,4-thiadiazol-2-yl)-1H-pyrazol-5-yl)acetamide (product of Step 3, Example 5; “ADT amine reagent”) (40 mg, 0.18 mmol, 1.1 equiv) and triethylamine (TEA) (33 mg, 0.33 mmol, 2 equiv) at room temperature and the resulting mixture was stirred for 1 h at room temperature. The mixture was then concentrated under reduced pressure and the resulting residue was purified by C18 reverse phase flash chromatography (mobile phase, MeCN in water (0.1% TFA), 35% to 42% gradient in 10 min; detector, UV 254 nm) to provide N-(5-(5-acetamido-1H-pyrazol-1-yl)-1,3,4-thiadiazol-2-yl)-4-(3,5-dimethoxypyridin-4-yl)-3-(2-methoxyethoxy)-2-oxo-2H-pyran-6-carboxamide (Compound 28-Ac) (40 mg, 42% yield). LCMS (ES, m/z)=558.1 [M+H]+.

Step 6: A solution of N-(5-(5-acetamido-1H-pyrazol-1-yl)-1,3,4-thiadiazol-2-yl)-4-(3,5-dimethoxypyridin-4-yl)-3-(2-methoxyethoxy)-2-oxo-2H-pyran-6-carboxamide (Compound 28-Ac) (56 mg, 0.10 mmol, 1 equiv) in conc. HCl (2 mL) was stirred for 4 h at room temperature. The mixture was then concentrated under reduced pressure and the resulting residue was purified by C18 reverse phase flash chromatography (mobile phase, MeCN in water (5 mmol/L NH₄HCO₃), 20% to 35% gradient in 10 min; detector, UV 254 nm) followed by additional purification by Prep-HPLC (X Bridge Prep OBD C18 Column, 30*150 mm; Mobile Phase A: water (10 mmol/L NH₄HCO₃), Mobile Phase B: acetonitrile (MeCN); Flow rate: 60 mL/min mL/min; Gradient: isocratic 44-54; Wave Length: 254 nm/220 nm) to afford N-(5-(5-amino-1H-pyrazol-1-yl)-1,3,4-thiadiazol-2-yl)-4-(3,5-dimethoxypyridin-4-yl)-3-(2-methoxyethoxy)-2-oxo-2H-pyran-6-carboxamide (Compound 28) (9.0 mg, 17% yield). LCMS (ES, m/z)=516.1 [M+H]+; ¹H NMR (400 MHz, DMSO-d6) δ 8.22 (s, 2H), 7.33 (d, J=1.6 Hz, 1H), 6.88 (s, 1H), 6.60 (s, 2H), 5.38 (d, J=1.6 Hz, 1H), 4.15 (t, J=4.8 Hz, 2H), 3.90 (s, 6H), 3.38 (t, J=4.8 Hz, 2H), 3.09 (s, 3H).

### Example 24: N-(5-(5-amino-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 29) and N-(5-(5-acetamido-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 29-Ac)

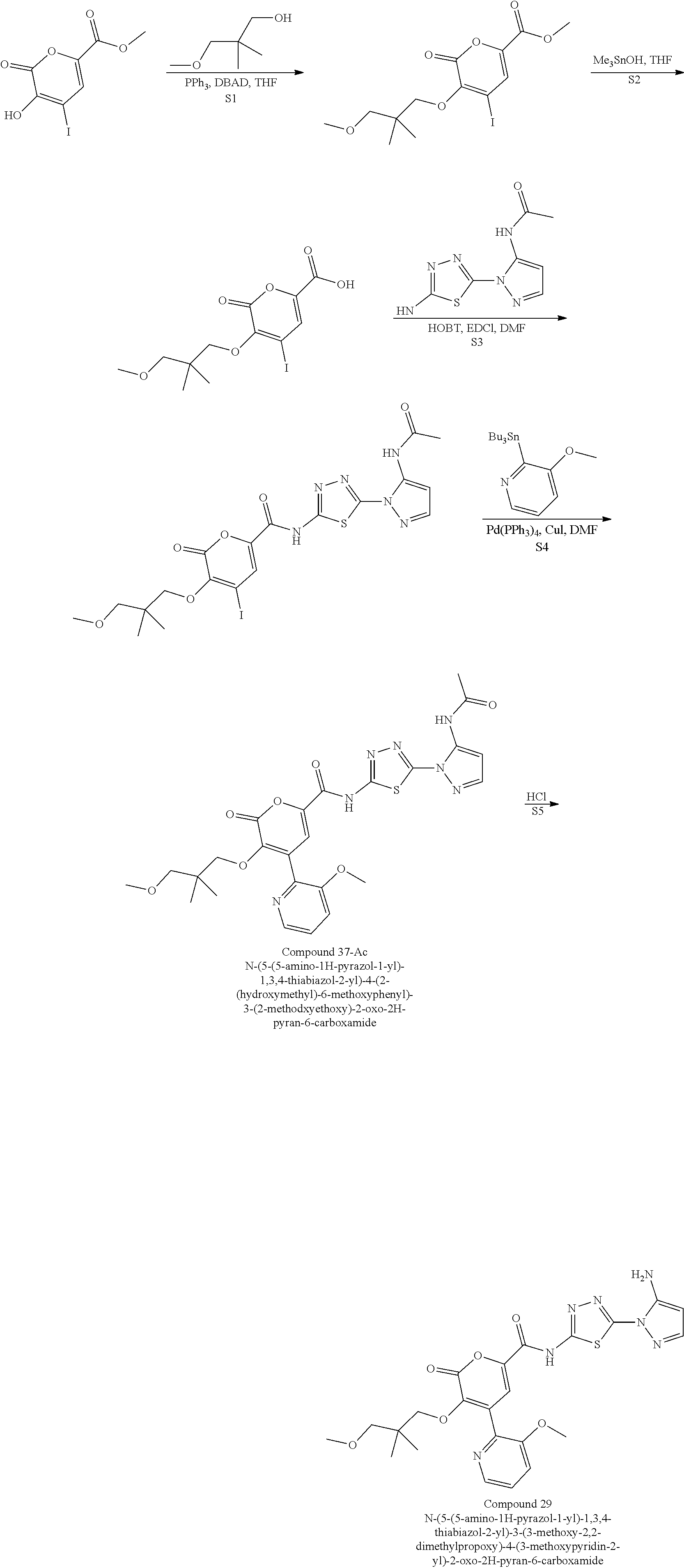

Step 1: To a stirred solution of methyl 5-hydroxy-4-iodo-6-oxopyran-2-carboxylate (product of Step 1, Example 11) (1 g, 3.4 mmol, 1 equiv), 3-methoxy-2,2-dimethylpropan-1-ol (0.48 g, 4.05 mmol, 1.2 equiv) and triphenyl phosphine (PPh₃) (1.33 g, 5.07 mmol, 1.5 equiv) in tetrahydrofuran (THF) (10 mL) was added di-tert-butyl azodicarboxylate (DBAD) (1.56 g, 6.76 mmol, 2 equiv) in portions at 0° C. under nitrogen atmosphere. The resulting mixture was stirred overnight at room temperature under nitrogen atmosphere then quenched with water (50 mL) at room temperature and extracted with ethyl acetate (EtOAc) (3×50 mL). The combined organic layers were dried over anhydrous Na₂SO₄, filtered, and concentrated under reduced pressure. The resulting residue was purified by silica gel column chromatography (eluting with petroleum ether/ethyl acetate (PE/EtOAc) (10:1)) to afford methyl 4-iodo-5-(3-methoxy-2,2-dimethylpropoxy)-6-oxopyran-2-carboxylate (700 mg, 52% yield). LCMS (ES, m/z)=397.2 [M+H]+.

Step 2: Into a solution of methyl 4-iodo-5-(3-methoxy-2,2-dimethylpropoxy)-6-oxopyran-2-carboxylate (400 mg, 1.01 mmol, 1 equiv) in tetrahydrofuran (THF) (8 mL) was added trimethylstannanol (366 mg, 2.02 mmol, 2 equiv). The resulting mixture was stirred for 1 h at 40° C. then cooled to rt and quenched with water (20 mL). The solution was extracted with ethyl acetate (EtOAc) (3×40 mL) and the combined organic layers were dried over anhydrous Na₂SO₄, filtered, and concentrated under reduced pressure. The resulting residue was purified by C18 reverse phase flash chromatography (mobile phase, MeCN in water (0.1% TFA), 10% to 30% gradient in 10 min; detector, UV 254 nm) to afford 4-iodo-5-(3-methoxy-2,2-dimethylpropoxy)-6-oxopyran-2-carboxylic acid (400 mg, 98% yield). LCMS (ES, m/z)=383.1 [M+H]+.

Step 3: To a stirred solution of 4-iodo-5-(3-methoxy-2,2-dimethylpropoxy)-6-oxopyran-2-carboxylic acid (400 mg, 1.05 mmol, 1 equiv) in N,N-dimethylformamide (DMF) (8 mL) was added hydroxybenzotriazole (HOBT) (283 mg, 2.09 mmol, 2 equiv), l-ethyl-3-(3-dimethylaminopropyl)carbodiimide (EDCI) (402 mg, 2.10 mmol, 2 equiv) and N-(1-(5-amino-1,3,4-thiadiazol-2-yl)-1H-pyrazol-5-yl)acetamide (product of Step 3, Example 5) (305 mg, 1.36 mmol, 1.30 equiv) at room temperature. The resulting mixture was stirred for 2 h at room temperature then quenched with water (20 mL). The solution was extracted with ethyl acetate (EtOAc) (3×30 mL), and the combined organic layers were dried over anhydrous Na₂SO₄, filtered, and concentrated under reduced pressure. The resulting residue was purified by silica gel column chromatography (eluting with CH₂Cl₂/MeOH (12:1)) to afford N-[5-(5-acetamidopyrazol-1-yl)-1,3,4-thiadiazol-2-yl]-4-iodo-5-(3-methoxy-2,2-dimethylpropoxy)-6-oxopyran-2-carboxamide (300 mg, 49% yield). LCMS (ES, m/z)=589.4 [M+H]+.

Step 4: A solution of N-[5-(5-acetamidopyrazol-1-yl)-1,3,4-thiadiazol-2-yl]-4-iodo-5-(3-methoxy-2,2-dimethylpropoxy)-6-oxopyran-2-carboxamide (“halo-pyrone reagent”) (200 mg, 0.34 mmol, 1 equiv), 3-methoxy-2-(tributylstannyl)pyridine (“aryl tin reagent”) (259 mg, 0.65 mmol, 2 equiv), tetrakis (triphenylphosphine)palladium (0) (Pd(PPh₃)₄) (78 mg, 0.067 mmol, 0.2 equiv), CuI (20 mg, 0.105 mmol, 0.31 equiv) and CsF (100 mg, 0.66 mmol, 1.94 equiv) in N,N-dimethylformamide (DMF) (5 mL) was stirred for 1 h at 100° C. under nitrogen atmosphere. The reaction was then cooled to rt and quenched by the addition of saturated aqueous KF (20 mL), and extracted with ethyl acetate (EtOAc) (3×50 mL). The combined organic layers were dried over anhydrous Na₂SO₄, filtered, and concentrated under reduced pressure. The resulting residue was purified by C18 reverse phase flash chromatography (mobile phase, MeCN in water (0.1% TFA), 10% to 50% gradient in 10 min; detector, UV 254 nm) to afford N-(5-(5-acetamido-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 29-Ac) (60 mg, 31% yield). LCMS (ES, m/z)=570.3 [M+H]+.

Step 5: A solution of N-(5-(5-acetamido-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 29-Ac) (60 mg, 0.105 mmol, 1 equiv) in conc. HCl (5 mL) was stirred for 6 h at room temperature. The mixture was then concentrated under vacuum and the residue was purified by C18 reverse phase flash chromatography (C18-M Column 20-35 μm 100Å 48 g; Mobile Phase A: water (50 mmol/L TFA), Mobile Phase B: acetonitrile (MeCN); Flow rate: 25 mL/min mL/min: Gradient: 30% B to 50% B in 20 min; Wave Length: 254 nm/220 nm nm) to afford N-(5-(5-amino-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 29) (27 mg, 48% yield). LCMS (ES, m/z)=528.1 [M+H]+; ¹H NMR (400 MHz, DMSO-d6) δ 13.30 (s, 1H), 8.31 (d, J=4.4 Hz, 1H), 7.66 (d, J=8.8 Hz, 1H), 7.54-7.48 (m, 3H), 6.74-6.65 (m, 2H), 5.45 (d, J=2.0 Hz, 1H), 3.86 (s, 5H), 3.07 (s, 3H), 2.82 (s, 2H), 0.67 (s, 6H).

### Example 25: 4-(4-cyano-1-methyl-1H-pyrazol-3-yl)-3-methoxy-N-(5-(5-methyl-1H-pyrazol-1-yl)-1,3,4-thiadiazol-2-yl)-2-oxo-2H-pyran-6-carboxamide (Compound 31) and 4-(4-iodo-1-methyl-1H-pyrazol-3-yl)-3-methoxy-N-(5-(5-methyl-1H-pyrazol-1-yl)-1,3,4-thiadiazol-2-yl)-2-oxo-2H-pyran-6-carboxamide (Compound 16)

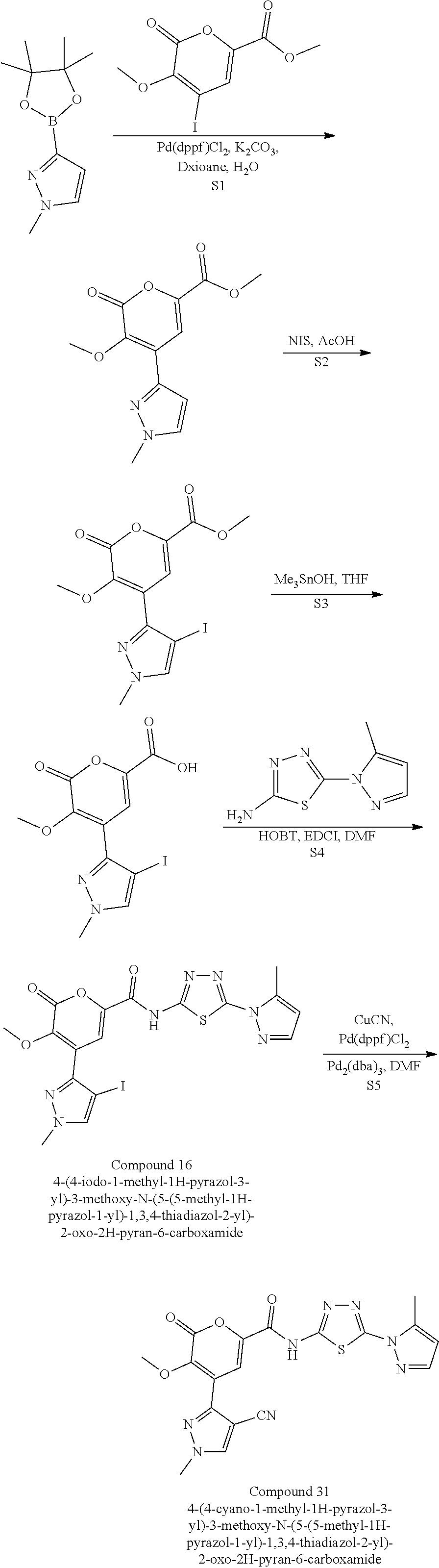

Step 1: A solution of 1-methyl-3-(4,4,5,5-tetramethyl-1,3,2-dioxaborolan-2-yl)pyrazole (1000 mg, 4.806 mmol, 1.5 equiv), methyl 4-iodo-5-methoxy-6-oxopyran-2-carboxylate (1000 mg, 3.225 mmol, 1 equiv), [1,1′-bis(diphenylphosphino)ferrocene]dichloropalladium(II) (Pd(dppf)Cl₂) (472 mg, 0.64 nmol, 0.2 equiv) and K₂CO₃ (1337 mg, 9.674 mmol, 3 equiv) in dioxane (24 mL) and H₂O (4 mL) was stirred for 2 h at 80° C. under nitrogen atmosphere. The mixture was then cooled to rt and quenched with water (50 mL), extracted with ethyl acetate (EtOAc) (3×50 mL), and the combined organic layers dried over anhydrous Na₂SO₄, filtered, and concentrated under reduced pressure. The resulting residue was purified by silica gel column chromatography (eluting with CH₂Cl₂/MeOH (10:1)) to afford methyl 5-methoxy-4-(1-methylpyrazol-3-yl)-6-oxopyran-2-carboxylate (800 mg, 94% yield). LCMS (ES, m/z)=265.2 [M+H]+.

Step 2: To a solution of methyl 5-methoxy-4-(1-methylpyrazol-3-yl)-6-oxopyran-2-carboxylate (800 mg, 3.03 mmol, 1 equiv) in acetic acid (AcOH) (12 mL) was added N-iodosuccinimide (NIS) (1362 mg, 6.054 mmol, 2 equiv). The resulting solution was stirred for 3 h at 40° C. then cooled to rt and diluted with ethyl acetate (EtOAc) (200 mL). The solution was washed with water (3×20 mL), dried over anhydrous Na₂SO₄, filtered, and concentrated under reduced pressure. The resulting residue was purified by silica gel column chromatography (eluting with petroleum ether/ethyl acetate (PE/EtOAc) (1:1)) to afford methyl 4-(4-iodo-1-methylpyrazol-3-yl)-5-methoxy-6-oxopyran-2-carboxylate (800 mg, 68% yield). LCMS (ES, m/z)=390.9 [M+H]+.

Step 3: To a solution of methyl 4-(4-iodo-1-methylpyrazol-3-yl)-5-methoxy-6-oxopyran-2-carboxylate (300 mg, 0.77 mmol, 1 equiv) in tetrahydrofuran (THF) (6 mL) was added trimethylstannanol (279 mg, 1.54 mmol, 2.01 equiv) and the resulting mixture was stirred for 5 h at 40° C. The mixture was then cooled to rt and concentrated under vacuum and the crude residue purified by C18 reverse phase flash chromatography (mobile phase, MeCN in water (0.1% TFA), 10% to 30% gradient in 10 min: detector. UV 254 nm) to afford 4-(4-iodo-1-methylpyrazol-3-yl)-5-methoxy-6-oxopyran-2-carboxylic acid (210 mg, 73% yield). LCMS (ES, m/z)=377.1 [M+H]+.

Step 4: To a stirred solution of 4-(4-iodo-1-methylpyrazol-3-yl)-5-methoxy-6-oxopyran-2-carboxylic acid (“aryl-pyrone reagent”) (200 mg, 0.53 mmol, 1 equiv), hydroxybenzotriazole (HOBT) (143 mg, 1.06 mmol, 1.99 equiv) and 1-ethyl-3-(3-dimethylaminopropyl)carbodiimide (EDCI) (207 mg, 1.08 mmol, 2.03 equiv) in N,N-dimethylformamide (DMF) (5 mL) was added 5-(5-methylpyrazol-1-yl)-1,3,4-thiadiazol-2-amine (product of Step 8, Example 1; “ADT amine reagent”) (116 mg, 0.64 mmol, 1.20 equiv) at room temperature. The resulting mixture was stirred for 1 h at room temperature then water (10 mL) was added. The resulting solids were collected by filtration and washed with water (3×1 mL) and acetonitrile (MeCN) (2×1 mL) to afford 4-(4-iodo-1-methyl-1H-pyrazol-3-yl)-3-methoxy-N-(5-(5-methyl-1H-pyrazol-1-yl)-1,3,4-thiadiazol-2-yl)-2-oxo-2H-pyran-6-carboxamide (Compound 16) (300 mg, 100% yield). LCMS (ES, m/z)=540.3 [M+H]+.

Step 5: A mixture of 4-(4-iodo-1-methyl-1H-pyrazol-3-yl)-3-methoxy-N-(5-(5-methyl-1H-pyrazol-1-yl)-1,3,4-thiadiazol-2-yl)-2-oxo-2H-pyran-6-carboxamide (Compound 16) (100 mg, 0.18 mmol, 1 equiv), CuCN (32 mg, 0.36 mmol, 1.93 equiv), [1,1′-bis(diphenylphosphino)ferrocene]dichloro palladium(II) (Pd(dppf)Cl₂) (14 mg, 0.019 mmol, 0.1 equiv) and tris(dibenzylidenaceton)dipalladium(0) dibenzylidenacetone (Pd₂(dba)₃) (17 mg, 0.019 mmol, 0.1 equiv) in N,N-dimethylformamide (DMF) (1 mL) was stirred for 4 h at 150° C. under nitrogen atmosphere. The reaction mixture was then cooled to rt and quenched with water (20 mL). The solution was extracted with ethyl acetate (EtOAc) (3×40 mL), and the combined organic layers dried over anhydrous Na₂SO₄, filtered, and concentrated under reduced pressure. The resulting residue was purified by C18 reverse phase flash chromatography (Flash C18-M Column 20-35 m 100Å 48 g; Mobile Phase A: water (50 mmol/L TFA), Mobile Phase B: acetonitrile (MeCN): Flow rate: 25 mL/min mL/min; Gradient: 30/6 B to 50% B in 20 min; Wave Length: 254 nm/220 nm) to afford 4-(4-cyano-1-methyl-1H-pyrazol-3-yl)-3-methoxy-N-(5-(5-methyl-1H-pyrazol-1-yl)-1,3,4-thiadiazol-2-yl)-2-oxo-2H-pyran-6-carboxamide (Compound 31) (4.4 mg, 5.2% yield). LCMS (ES, m/z)=439.1 [M+H]+; ¹H NMR (400 MHz, DMSO-d6) δ 8.73 (s, 1H), 7.78-7.73 (m, 2H), 6.44 (s, 1H), 4.10 (s, 3H), 4.01 (s, 3H), 2.66 (s, 3H).

### Example 26: N-(5-(5-amino-1H-pyrazol-1-yl)-1,3,4-thiadiazol-2-yl)-4-(2-(cyanomethyl)-6-methoxyphenyl)-3-(2-methoxyethoxy)-2-oxo-2H-pyran-6-carboxamide (Compound 32), N-(5-(5-acetamido-1H-pyrazol-1-yl)-1,3,4-thiadiazol-2-yl)-4-(2-(cyanomethyl)-6-methoxyphenyl)-3-(2-methoxyethoxy)-2-oxo-2H-pyran-6-carboxamide (Compound 32-Ac), and N-(5-(5-acetamido-1H-pyrazol-1-yl)-1,3,4-thiadiazol-2-yl)-4-(2-(hydroxymethyl)-6-methoxyphenyl)-3-(2-methoxyethoxy)-2-oxo-2H-pyran-6-carboxamide (Compound 37-Ac)

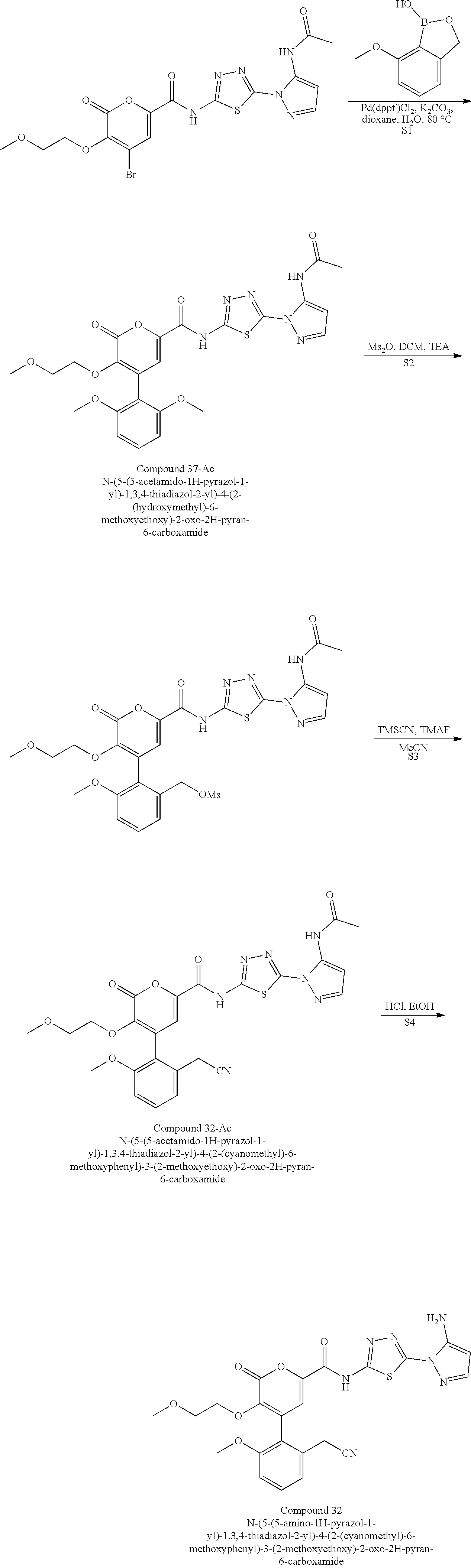

Step 1: A solution of 4-bromo-N-[5-(5-acetamidopyrazol-1-yl)-1,3,4-thiadiazol-2-yl]-5-(2-methoxyethoxy)-6-oxopyran-2-carboxamide (product of Step 4, Example 5; (“halo-pyrone reagent”)) (300 mg, 0.601 mmol, 1 equiv), [1,1′-bis(diphenylphosphino)ferrocene]dichloro palladium(II) (Pd(dppf)Cl₂) CH₂Cl₂ complex (88 mg, 0.11 mmol, 0.2 equiv), K₂CO₃ (254 mg, 1.84 mmol, 3 equiv) and 7-methoxy-3H-2,1-benzoxaborol-1-ol (“aryl boron reagent”) (120 mg, 0.732 mmol, 1.2 equiv) in dioxane (6 mL) and H₂O (1 mL) was stirred for 1 h at 80° C. under nitrogen atmosphere. The mixture was then cooled to rt and concentrated under vacuum, and the residue was purified by silica gel column chromatography (eluting with CH₂Cl₂/MeOH (10:1)) to afford N-(5-(5-acetamido-1H-pyrazol-1-yl)-1,3,4-thiadiazol-2-yl)-4-(2-(hydroxymethyl)-6-methoxyphenyl)-3-(2-methoxyethoxy)-2-oxo-2H-pyran-6-carboxamide (Compound 37-Ac) (150 mg, 45% yield). LCMS (ES, m/z)=557.1 [M+H]+.

Step 2: To a solution of N-(5-(5-acetamido-1H-pyrazol-1-yl)-1,3,4-thiadiazol-2-yl)-4-(2-(hydroxymethyl)-6-methoxyphenyl)-3-(2-methoxyethoxy)-2-oxo-2H-pyran-6-carboxamide (Compound 37-Ac) (150 mg, 0.27 mmol, 1 equiv) in dichloromethane (DCM) (4 mL) was added methanesulfonyl methanesulfonate (240 mg, 1.38 mmol, 5 equiv) and triethylamine (TEA) (80 mg, 0.79 mmol, 3 equiv) and the solution was stirred for 2 h at room temperature. The mixture was then concentrated under reduced pressure and the resulting residue was purified by silica gel column chromatography (eluting with CH₂Cl₂/MeOH (10:1)) to afford [2-(6-{[5-(5-acetamidopyrazol-1-yl)-1,3,4-thiadiazol-2-yl]carbamoyl)-3-(2-methoxyethoxy)-2-oxopyran-4-yl}-3-methoxyphenyl]methyl methanesulfonate (110 mg, 64% yield). LCMS (ES, m/z)=635.1 [M+H]+.

Step 3: To a solution of [2-(6-(15-(5-acetamidopyrazol-1-yl)-1,3,4-thiadiazol-2-yl]carbamoyl-3-(2-methoxyethoxy)-2-oxopyran-4-yl)-3-methoxyphenylmethyl methanesulfonate (100 mg, 0.16 mmol, 1 equiv) in acetonitrile (MeCN) (5 mL) was added tetramethylammonium fluoride (TMAF) (59 mg, 0.633 mmol, 4 equiv) and trimethylsilyl cyanide (TMSCN) (188 mg, 1.90 mmol, 12 equiv). The resulting solution was stirred for 3 h at 80° C. then cooled to rt and concentrated under reduced pressure. The resulting residue was purified by silica gel column chromatography (eluting with CH₂Cl₂/MeOH (10:1)) to afford N-(5-(5-acetamido-1H-pyrazol-1-yl)-1,3,4-thiadiazol-2-yl)-4-(2-(cyanomethyl)-6-methoxyphenyl)-3-(2-methoxyethoxy)-2-oxo-2H-pyran-6-carboxamide (Compound 32-Ac) (80 mg, 90% yield). LCMS (ES, m/z)=566.1 [M+H]+.

Step 4: To a solution of N-(5-(5-acetamido-1H-pyrazol-1-yl)-1,3,4-thiadiazol-2-yl)-4-(2-(cyanomethyl)-6-methoxyphenyl)-3-(2-methoxyethoxy)-2-oxo-2H-pyran-6-carboxamide (Compound 32-Ac) (80 mg, 0.14 mmol, 1 equiv) in ethanol (EtOH) (1 mL) was added conc. HCl (1 mL). The resulting solution was stirred for 3 h at room temperature then concentrated under reduced pressure. The resulting residue was purified by C18 reverse phase flash chromatography (mobile phase, MeCN in water (0.1% TFA), 10% to 50% gradient in 10 min; detector, UV 254 nm) followed by additional purification by Prep-HPLC (X Bridge Prep OBD C18 Column, 30*150 mm; Mobile Phase A: water (10 mmol/L NH₄HCO₃), Mobile Phase B: acetonitrile (MeCN); Flow rate: 60 mL/min mL/min; Gradient: 14% B to 24% B in 7.8 min, Wave Length: 254 nm/220 nm nm) to afford N-(5-(5-amino-1H-pyrazol-1-yl)-1,3,4-thiadiazol-2-yl)-4-(2-(cyanomethyl)-6-methoxyphenyl)-3-(2-methoxyethoxy)-2-oxo-2H-pyran-6-carboxamide (Compound 32) (2.6 mg, 3.4% yield). LCMS (ES, m/z)=524.2 [M+H]+: ¹H NMR (400 MHz, DMSO-d6) δ 7.48 (dd. J=8.0, 1.2 Hz, 1H), 7.33 (s, 1H), 7.15 (s, 1H), 7.13 (s, 1H), 6.85 (s, 1H), 6.59 (s, 2H), 5.38 (d, J=1.6 Hz, 1H), 4.18-4.14 (m, 1H), 4.09-4.05 (m, 1H), 3.83-3.78 (m, 2H), 3.76 (s, 3H), 3.34-3.31 (m, 2H), 3.03 (s, 3H).

### Example 27: N-(5-(5-amino-1H-pyrazol-1-yl)-1,3,4-thiadiazol-2-yl)-3-(3-hydroxy-2,2-dimethylpropoxy)-4-(3-methoxypyridin-2-yl)-2-oxo-2H-pyran-6-carboxamide (Compound 33)

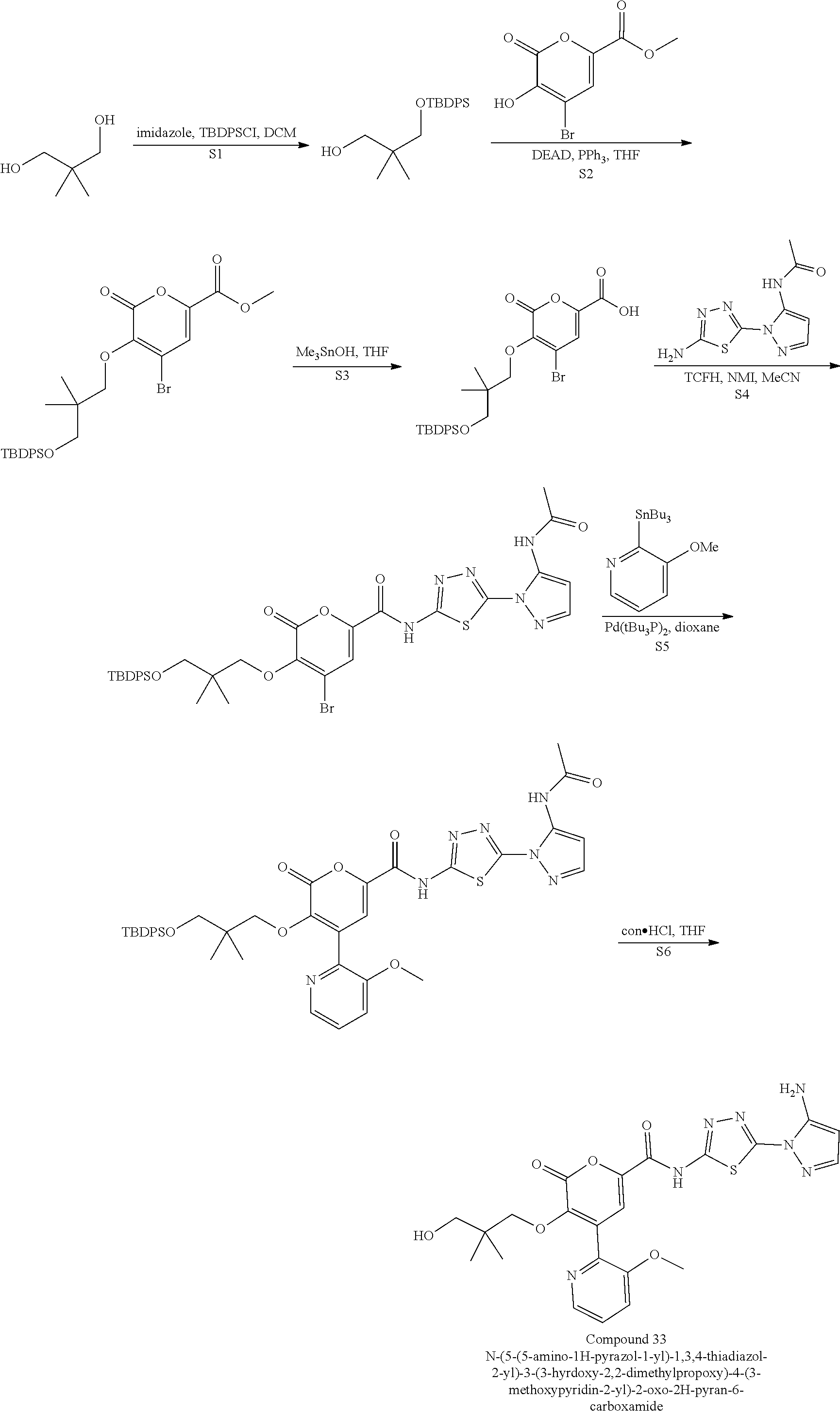

Step 1: To a solution of 2,2-dimethylpropane-1,3-diol (1 g, 9.62 mmol, 1 equiv) in dichloromethane (DCM) (20 mL) was added imidazole (1.96 g, 28.8 mmol, 3 equiv) and tert-butyl(chloro)diphenylsilane (TBDPSCl) (2.63 g, 9.60 mmol, 1 equiv). The resulting solution was stirred at rt for 3 h then washed with water and concentrated under reduced pressure. The resulting residue was purified by silica gel column chromatography (eluting with petroleum ether/ethyl acetate (PE/EtOAc) (5:1)) to afford 3-((tert-butyldiphenylsilyl)oxy)-2,2-dimethylpropan-1-ol (1.97 g, 60% yield). LCMS (ES, m/z)=343.1 [M+H]+.

Step 2: To a solution of 3-[(tert-butyldiphenylsilyl)oxy]-2,2-dimethylpropan-1-ol (300 mg, 0.88 mmol, 1 equiv), methyl 4-bromo-5-hydroxy-6-oxopyran-2-carboxylate (product of Step 2, Example 1) (261 mg, 1.05 mmol, 1.2 equiv) and triphenyl phosphine (PPh₃) (344 mg, 1.31 mmol, 1.5 equiv) in tetrahydrofuran (THF) (10 mL) was added diethyl azodicarboxylate (DEAD) (228 mg, 1.31 mmol, 1.5 equiv) at 0° C. The mixture was then stirred for 1 h at 80° C. then cooled to rt and concentrated under reduced pressure. The resulting residue was purified by silica gel column chromatography (eluting with petroleum ether/ethyl acetate (PE/EtOAc) (5:1)) to afford methyl 4-bromo-5-{3-[(tert-butyldiphenylsilyl)oxy]-2,2-dimethylpropoxy}-6-oxopyran-2-carboxylate (500 mg, 99% yield). LCMS (ES, m/z)=573.1 [M+H]+.

Step 3: To a solution of methyl 4-bromo-5-{3-[(tert-butyldiphenylsilyl)oxy]-2,2-dimethylpropoxy}-6-oxopyran-2-carboxylate (500 mg, 0.87 mmol, 1 equiv) in tetrahydrofuran (THF) (10 mL) was added trimethylstannylhydroxide (315 mg, 1.74 mmol, 2 equiv). The resulting mixture was stirred for 2 h at room temperature then concentrated under reduced pressure. The resulting residue was purified by C18 reverse phase flash chromatography (mobile phase, MeCN in water (0.1% HCl), 0% to 87% gradient in 20 min; detector, UV 220 nm) to provide 4-bromo-5-{3-[(tert-butyldiphenylsilyl)oxy]-2,2-dimethylpropoxy}-6-oxopyran-2-carboxylic acid (400 mg, 82% yield). LCMS (ES, m/z)=559.1 [M+H]+.

Step 4: To a solution of 4-bromo-5-{3-[(tert-butyldiphenylsilyl)oxy]-2,2-dimethylpropoxy}-6-oxopyran-2-carboxylic acid (400 mg, 0.72 mmol, 1 equiv) and N-(1-(5-amino-1,3,4-thiadiazol-2-yl)-1H-pyrazol-5-yl)acetamide (product of Step 3, Example 5) (176 mg, 0.79 mmol, 1.1 equiv) in acetonitrile (MeCN) (5 mL) was added chloro-N,N,N′,N′-tetramethylformamidinium hexafluorophosphate (TCFH) (300 mg, 1.07 mmol, 1.5 equiv) and N-methylimidazole (NMI) (176 mg, 2.14 mmol, 3 equiv). The mixture was stirred for 1 h at room temperature then the resulting solids collected by filtration and purified by C18 reverse phase flash chromatography (mobile phase. MeCN in water (0.1% HCl), 0% to 100% gradient in 20 min; detector, UV 220 nm) to provide 4-bromo-5-{3-[(tert-butyldiphenylsilyl)oxy]-2,2-dimethylpropoxy}-N-[5-(5-acetamidopyrazol-1-yl)-1,3,4-thiadiazol-2-yl]-6-oxopyran-2-carboxamide (320 mg, 58% yield). LCMS (ES, m/z)=765.1 [M+H]+.

Step 5: To a solution of 4-bromo-5-{3-[(tert-butyldiphenylsilyl)oxy]-2,2-dimethylpropoxy}-N-[5-(5-acetamidopyrazol-1-yl)-1,3,4-thiadiazol-2-yl]-6-oxopyran-2-carboxamide (“halo-pyrone reagent”) (300 mg, 0.392 mmol, 1 equiv), 3-methoxy-2-(tributylstannyl)pyridine (“aryl tin reagent”) (234 mg, 0.588 mmol, 1.5 equiv) and bis(tri-tert-butylphosphine)palladium(0) (Pd(t-Bu₃P)₂) (40 mg, 0.078 mmol, 0.20 equiv) in 1,4-dioxane (5 mL) was stirred for 1 h at 80° C. under nitrogen atmosphere. The mixture was then cooled to rt and concentrated under reduced pressure. The resulting residue was purified by C18 reverse phase flash chromatography (mobile phase, MeOH in water (0.1% HCl), 0% to 70% gradient in 20 min; detector, UV 220 nm) to provide 5-{3-[(tert-butyldiphenylsilyl)oxy]-2,2-dimethylpropoxy}-N-[5-(5-acetamidopyrazol-1-yl)-1,3,4-thiadiazol-2-yl]-4-(3-methoxypyridin-2-yl)-6-oxopyran-2-carboxamide (60 mg, 19% yield). LCMS (ES, m/z)=794.1 [M+H]+.

Step 6: To a solution of 5-{3-[(tert-butyldiphenylsilyl)oxy]-2,2-dimethylpropoxy}-N-[5-(5 acetamidopyrazol-1-yl)-1,3,4-thiadiazol-2-yl]-4-(3-methoxypyridin-2-yl)-6-oxopyran-2-carboxamide (60 mg, 0.076 mmol, 1 equiv) in tetrahydrofuran (THF) (3 mL) was added conc. HCl (1.5 mL). The resulting solution was stirred for 1 h at 60° C. under nitrogen atmosphere then cooled to rt and concentrated under reduced pressure. The resulting residue was purified by C18 reverse phase flash chromatography (mobile phase, MeOH in water (0.1% HCl), 0% to 50% gradient in 20 min; detector, UV 254 nm) to provide N-(5-(5-amino-1H-pyrazol-1-yl)-1,3,4-thiadiazol-2-yl)-3-(3-hydroxy-2,2-dimethylpropoxy)-4-(3-methoxypyridin-2-yl)-2-oxo-2H-pyran-6-carboxamide (Compound 33) (3.5 mg, 9% yield). LCMS (ES, m/z)=514.1 [M+H]+; ¹H NMR (300 MHz, DMSO-d6) δ 8.28 (d, J=4.8 Hz, 1H), 7.61 (d, J=8.7 Hz, 1H), 7.50 (dd. J=8.4, 4.5 Hz, 1H), 7.40 (d, J=1.8 Hz, 1H), 7.21 (s, 1H), 5.44 (d, J=1.8 Hz, 1H), 3.85 (s, 3H), 3.78 (s, 2H), 2.99 (s, 2H), 0.61 (s, 6H).

### Example 28: (Sa)-N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 34A*), (Ra)—N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 34B*), and N-(5-(5-acetamido-1H-pyrazol-1-yl)-1,3,4-thiadiazol-2-yl)-3-(2-(tert-butoxy)ethoxy)-4-(2-chloro-6-methoxyphenyl)-2-oxo-2H-pyran-6-carboxamide (Compound 34-Ac-OtBu)

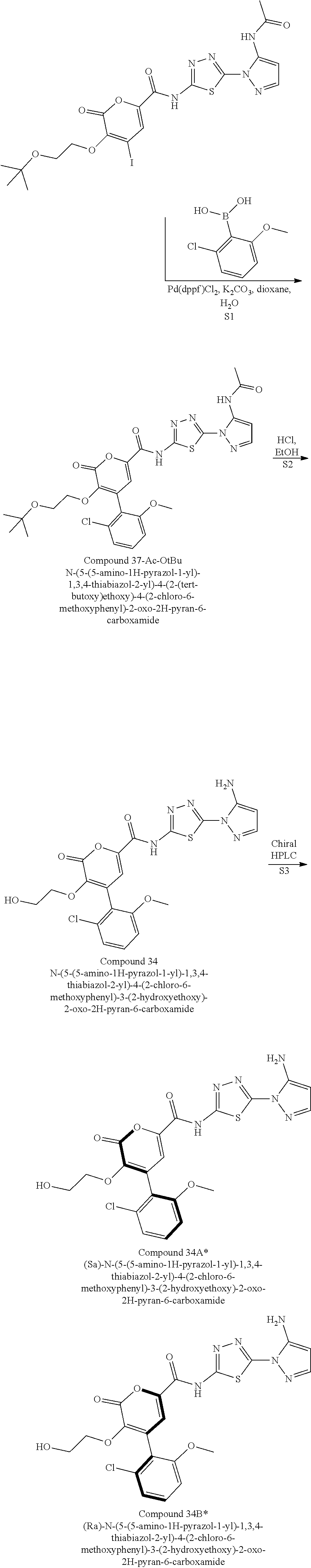

Step 1: To a stirred solution of 5-[2-(tert-butoxy)ethoxy]-N-[5-(5-acetamidopyrazol-1-yl)-1,3,4-thiadiazol-2-yl]-4-iodo-6-oxopyran-2-carboxamide (product of Step 4 of Example 13; “halo-pyrone reagent”) (100 mg, 0.017 mmol, 1 equiv) in N,N-dimethylformamide (DMF) (2 mL) and H₂O (0.5 mL) was added 2-chloro-6-methoxyphenylboronic acid (“aryl boron reagent”) (100 mg, 0.054 mmol, 3 equiv), [1,1′-bis(diphenylphosphino)ferrocene]dichloro palladium(II) (Pd(dppf)Cl₂) (30 mg, 0.004 mmol, 0.2 equiv) and K₂CO₃ (70 mg, 0.051 mmol, 3 equiv) at room temperature. The resulting mixture was stirred for 2 h at 80° C. under nitrogen atmosphere then the mixture was cooled to rt and purified directly by C18 reverse phase flash chromatography (mobile phase, MeCN in water (0.1% TFA), 10% to 50% gradient in 10 min; detector, UV 254 nm) to provide N-(5-(5-acetamido-1H-pyrazol-1-yl)-1,3,4-thiadiazol-2-yl)-3-(2-(tert-butoxy)ethoxy)-4-(2-chloro-6-methoxyphenyl)-2-oxo-2H-pyran-6-carboxamide (Compound 34-Ac-OtBu) (50 mg, 48% yield). LCMS (ES, m/z)=603.1 [M+1]+.

Steps 2-3: A solution of N-(5-(5-acetamido-1H-pyrazol-1-yl)-1,3,4-thiadiazol-2-yl)-3-(2-(tert-butoxy)ethoxy)-4-(2-chloro-6-methoxyphenyl)-2-oxo-2H-pyran-6-carboxamide (Compound 34-Ac-OtBu) (70 mg, 0.12 mmol, 1 equiv) in conc. HCl (1.40 mL) was stirred for 4 h at room temperature. The solution was then concentrated under reduced pressure and purified by C18 reverse phase flash chromatography (mobile phase. MeCN in water (0.1% TFA), 10% to 50% gradient in 10 min; detector, UV 254 nm) to provide N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 34). Separation of axially chiral atropisomers of N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide was performed by Prep Chiral HPLC (CHIRALPAK IF, 2*25 cm, 5 μm. Mobile Phase A: Hexanes (0.2% FA), Mobile Phase B: MeOH:DCM=1:1; Flow rate: 20 mL/min; Gradient: 80% B to 80% B in 9 min; Wave Length: 220/254 nm) to afford (Sa)-N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 34A*) (35 mg, 33% yield) as the first eluting peak (chiral RT=5.66 min), and (Ra)—N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 34B*) (25 mg, 24% yield) as the second eluting peak (chiral RT=7.70 min). Stereochemistry arbitrarily assigned.

Compound 34A*: LCMS (ES, m/z)=505.1 [M+H]+, ¹H NMR (400 MHz, DMSO-d6) δ 13.26 (br, 1H), 7.53-7.45 (m, 2H), 7.33 (s, 1H), 7.24-7.14 (m, 2H), 6.65 (s, 2H), 5.44 (d, J=2.0 Hz, 1H), 4.63 (s, 1H), 4.16-4.10 (m, 1H), 4.07-4.00 (m, 1H), 3.81 (s, 3H), 3.46-3.36 (m, 2H).

Compound 34B*: LCMS (ES, m/z)=505.1 [M+H]+, ¹H NMR (400 MHz, DMSO-d6) δ 13.26 (br, 1H), 7.51-7.47 (m, 2H), 7.31 (s, 1H), 7.22-7.15 (m, 2H), 6.65 (s, 2H), 5.44 (d, J=2.0 Hz, 1H), 4.64-4.62 (m, 1H), 4.18-4.13 (m, 1H), 4.09-4.04 (m, 1H), 3.81 (s, 3H), 3.46-3.36 (m, 2H).

### Example 29: N-(5-(5-amino-1H-pyrazol-1-yl)-1,3,4-thiadiazol-2-yl)-4-(2,6-dimethoxyphenyl)-3-(3-hydroxy-2,2-dimethylpropoxy)-2-oxo-2H-pyran-6-carboxamide (Compound 36)

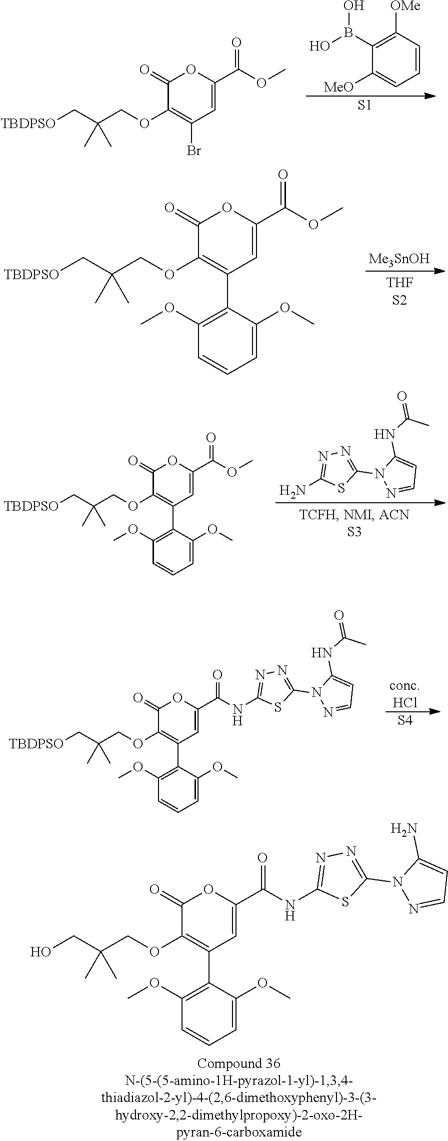

Step 1: A solution of methyl 4-bromo-5-{3-[(tert-butyldiphenylsilyl)oxy]-2,2-dimethylpropoxy}-6-oxopyran-2-carboxylate (product of Step 2 of Example 27) (560 mg, 0.976 mmol, 1 equiv), 2,6-dimethoxyphenylboronic acid (355 mg, 1.952 mmol, 2 equiv), [1,1′-bis(diphenylphosphino)ferrocene]dichloro palladium(II) (Pd(dppf)Cl₂)—CH₂Cl₂ complex (159 mg, 0.195 mmol, 0.2 equiv) and K₂CO₃ (404 mg, 2.928 mmol, 3 equiv) in 1,4-dioxane (10 mL) and H₂O (2 mL) was stirred for 2 h at 80° C. under nitrogen atmosphere. The mixture was then cooled to rt and concentrated under reduced pressure and the residue was purified by C18 reverse phase flash chromatography (mobile phase, MeCN in water (10 mmol/L NH₄HCO₃), 0% to 36% gradient in 10 min; detector, UV 254 nm) to provide methyl 5-{3-[(tert-butyldiphenylsilyl)oxy]-2,2-dimethylpropoxy}-4-(2,6-dimethoxyphenyl)-6-oxopyran-2-carboxylate (420 mg, 68% yield). LCMS (ES, m/z)=631.1 [M+H]+.

Step 2: To a solution of methyl 5-{3-[(tert-butyldiphenylsilyl)oxy]-2,2-dimethylpropoxy}-4-(2,6-dimethoxyphenyl)-6-oxopyran-2-carboxylate (490 mg, 0.777 mmol, 1 equiv) in tetrahydrofuran (THF) (10 mL) was added Me₃SnOH (1.21 g, 1.554 mmol, 2equiv). The resulting mixture was stirred for 2 h at room temperature under nitrogen atmosphere. The mixture was then concentrated under reduced pressure and the residue purified by C18 reverse phase flash chromatography (conditions: column, C18; mobile phase, MeCN in Water (0.1% HCl), 0% to 97% gradient in 20 min; detector, UV 220 nm) to provide 5-{3-[(tert-butyldiphenylsilyl)oxy]-2,2-dimethylpropoxy}-4-(2,6-dimethoxyphenyl)-6-oxopyran-2-carboxylic acid (440 mg, 92% yield). LCMS (ES, m/z)=617.1 [M+H]+.

Step 3: To a solution of 5-{3-[(tert-butyldiphenylsilyl)oxy]-2,2-dimethylpropoxy}-4-(2,6-dimethoxyphenyl)-6-oxopyran-2-carboxylic acid (“aryl-pyrone reagent”) (440 mg, 0.713 mmol, 1 equiv) and N-(1-(5-amino-1,3,4-thiadiazol-2-yl)-1H-pyrazol-5-yl)acetamide (product of Step 3, Example 5; “ADT amine reagent”) (239 mg, 1.069 mmol, 1.5 equiv) in acetonitrile (MeCN) (10 mL) was added chloro-N,N,N′,N′-tetramethylformamidinium hexafluorophosphate (TCFH) (300 mg, 1.069 mmol, 1.5 equiv) and N-methylimidazole (NMI) (234 mg, 2.85 mmol, 4 equiv). The mixture was stirred for 2 h at room temperature then the resulting solids were collected and purified by C18 reverse phase flash chromatography (mobile phase, MeOH in water (0.1% HCl), 0% to 41% gradient in 20 min; detector. UV 254 run) to provide 5-{3-[(tert-butyldiphenylsilyl)oxy]-2,2-dimethylpropoxy}-4-(2,6-dimethoxyphenyl)-N-[5-(5-acetamidopyrazol-1-yl)-1,3,4-thiadiazol-2-yl]-6-oxopyran-2-carboxamide (190 mg, 32% yield). LCMS (ES, m/z)=823.1 [M+H]+.

Step 4: To a solution of 5-{3-[(tert-butyldiphenylsilyl)oxy]-2,2-dimethylpropoxy}-4-(2,6-dimethoxyphenyl)-N-[5-(5-acetamidopyrazol-1-yl)-1,3,4-thiadiazol-2-yl]-6-oxopyran-2-carboxamide (180 mg, 0.219 mmol, 1 equiv) in tetrahydrofuran (THF) (4 mL) was added conc. HCl (2 mL) and the mixture was stirred for 1 h at room temperature. The solution was then concentrated under reduced pressure and the resulting residue purified by C18 reverse phase flash chromatography (mobile phase. MeCN in water (0.1% TFA), 0% to 42% gradient in 10 min; detector. UV 254 nm) to provide N-(5-(5-amino-1H-pyrazol-1-yl)-1,3,4-thiadiazol-2-yl)-4-(2,6-dimethoxyphenyl)-3-(3-hydroxy-2,2-dimethylpropoxy)-2-oxo-2H-pyran-6-carboxamide (Compound 36) (42 mg, 35% yield). LCMS (ES, m/z)=543.1 [M+H]+; ¹H NMR (300 MHz, DMSO-d6) δ 7.48 (s, 1H), 7.445 (dd, J=8.1, 2.4 Hz, 1H), 7.28 (s, 1H), 6.79 (s, 1H), 6.76 (s, 1H), 5.55 (s, 1H), 3.76 (s, 8H), 2.97 (s, 2H), 0.59 (s, 6H).

### Example 30: (Sa)-N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 37A*) and (Ra)—N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 37B*)

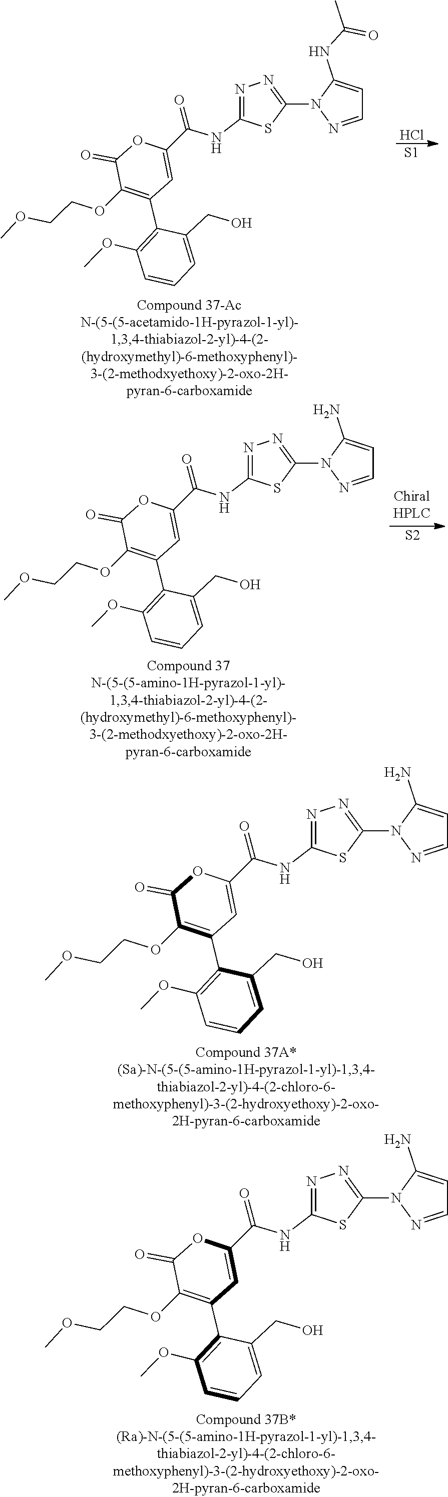

Step 1: A solution of N-(5-(5-acetamido-1H-pyrazol-1-yl)-1,3,4-thiadiazol-2-yl)-4-(2-(hydroxymethyl)-6-methoxyphenyl)-3-(2-methoxyethoxy)-2-oxo-2H-pyran-6-carboxamide (Compound 37-Ac; product of Step 1 of Example 26) (40 mg, 0.072 mmol, 1 equiv) in conc. HC (1 mL) was stirred for 3 h at room temperature. The mixture was concentrated under reduced pressure and the residue purified by C18 reverse phase flash chromatography (mobile phase. MeCN in water (0.1% TFA), 10% to 50% gradient in 10 min: detector. UV 254 nm) to give N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 37). LCMS (ES, m/z)=515.2 [M+H]+.

Step 2: Separation of axially chiral atropisomers of N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 37) was performed by Prep Chiral HPLC (CHIRAL ART Cellulose-SC, 2*25 cm; Mobile Phase A: Hexanes (0.2% FA), Mobile Phase B: EtOH:DCM=1:1; Flow rate: 20 mL/min: Gradient: 60% B to 60% B in 10 min; Wave Length: 220/254 nm) to afford (Sa)-N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 37A*) (5.1 mg, 13% yield) as the first eluting peak (chiral RT=7.35 min), and (Ra)—N-(5-(5-amino-1H-pyrazol-1-yl)-1,3,4-thiadiazol-2-yl)-4-(2-chloro-6-methoxyphenyl)-3-(2-hydroxyethoxy)-2-oxo-2H-pyran-6-carboxamide (Compound 37B*) (5.9 mg, 15% yield) as the second eluting peak (chiral RT=9.35 min). Stereochemistry arbitrarily assigned.

Compound 37A*: LCMS (ES, m/z)=515.2 [M+H]+, ¹H NMR (400 MHz, DMSO-d6) δ 13.22 (s, 1H), 7.45-7.40 (m, 2H), 7.24 (s, 1H), 7.17 (d, J=7.6 Hz, 1H), 7.04 (d, J=8.4 Hz, 1H), 6.62 (s, 2H), 5.43 (d, J=2.0 Hz, 1H), 5.11-5.00 (m, 1H), 4.33 (d, J=4.0 Hz, 2H), 4.19-4.15 (m, 1H), 4.07-3.94 (m, 1H), 3.76 (s, 3H), 3.33-3.29 (m, 2H), 3.04 (s, 3H).

Compound 37B*: LCMS (ES, m/z)=515.2 [M+H]+, ¹H NMR (400 MHz, DMSO-d6) δ 13.22 (s, 1H), 7.45-7.40 (m, 2H), 7.24 (s, 1H), 7.17 (d, J=7.6 Hz, 1H), 7.04 (d, J=8.4 Hz, 1H), 6.62 (s, 2H), 5.43 (d, J=2.0 Hz, 1H), 5.11-5.00 (m, 1H), 4.33 (d, J=4.0 Hz, 2H), 4.19-4.15 (m, 1H), 4.07-3.94 (m, 1H), 3.76 (s, 3H), 3.33-3.29 (m, 2H), 3.04 (s, 3H).

### Example 31: N-(5-(5-amino-1H-pyrazol-1-yl)-1,3,4-thiadiazol-2-yl)-4-(2-cyano-6-(methoxymethyl)phenyl)-3-(2-methoxyethoxy)-2-oxo-2H-pyran-6-carboxamide (Compound 39) and N-(5-(5-acetamido-1H-pyrazol-1-yl)-1,3,4-thiadiazol-2-yl)-4-(2-cyano-6-(methoxymethyl)phenyl)-3-(2-methoxyethoxy)-2-oxo-2H-pyran-6-carboxamide (Compound 39-Ac)

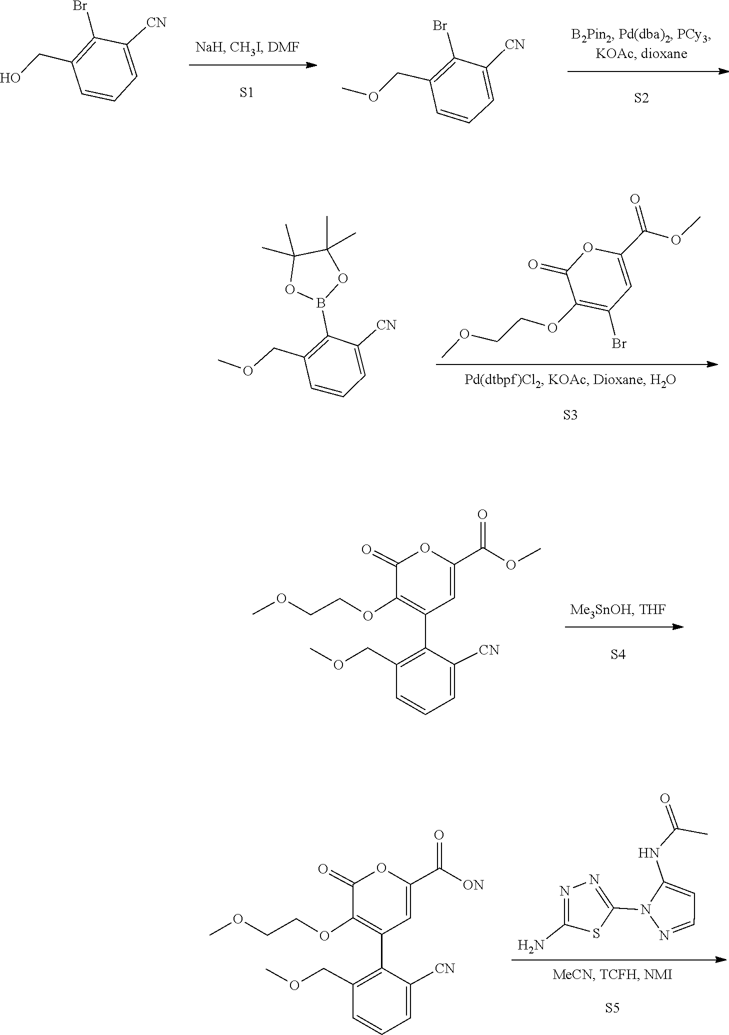

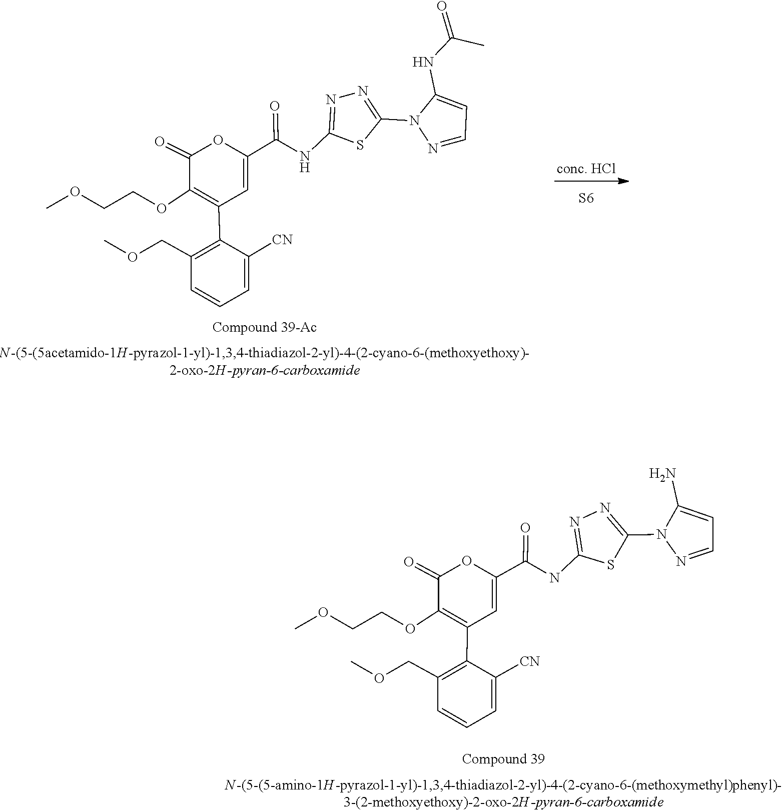

Step 1: To a solution of 2-bromo-3-(hydroxymethyl)benzonitrile (2 g, 9.4 mmol, 1 equiv) in tetrahydrofuran (THF) (80 mL) was added NaH (723 mg, 18.10 mmol, 1.92 equiv, 60% in mineral oil) at 0° C., and then the resulting mixture was stirred for 5 min at 0° C. To the above mixture was then added CH₃I (5.36 g, 37.7 mmol, 4equiv) at room temperature and the resulting mixture was stirred for 1 h at room temperature. The mixture was then quenched by the addition of Water/Ice (100 mL) at 0° C. and extracted with ethyl acetate (EtOAc) (3×200 mL). The combined organic layers were washed with brine (3×50 mL), dried over anhydrous Na₂SO₄, filtered, and concentrated under reduced pressure. The resulting residue was purified by silica gel column chromatography (eluting with petroleum ether/ethyl acetate (PE/EtOAc) (1:19)) to afford 2-bromo-3-(methoxymethyl)benzonitrile (1.6 g, 68% yield). LCMS (ES, m/z)=226.2 [M+H]+. ¹H NMR (400 MHz, DMSO-d6) δ 7.89 (dd, J=7.6, 1.6 Hz, 1H), 7.82-7.69 (m, 1H), 7.64-7.61 (m, 1H), 4.49 (s, 2H), 3.40 (s, 3H).

Step 2: 3-(methoxymethyl)-2-(4,4,5,5-tetramethyl-1,3,2-dioxaborolan-2-yl)benzonitrile was prepared according to Step 2 of Example 22 using 2-bromo-3-(methoxymethyl)benzonitrile in place of 2-bromo-1-methoxy-3-(2-methoxyethoxy) benzene. LCMS (ES, m/z)=273.9 [M+H]+.

Step 3: To a solution of 3-(methoxymethyl)-2-(4,4,5,5-tetramethyl-1,3,2-dioxaborolan-2-yl)benzonitrile (800 mg, 2.93 mmol, 1 equiv) in dioxane (18 mL) and H₂O (3 mL) was added methyl 4-bromo-5-(2-methoxyethoxy)-6-oxopyran-2-carboxylate (product of Step 1, Example 3) (900 mg, 2.93 mmol, 1 equiv) and [1,1′-bis(di-tert-butylphosphino)ferrocene]dichloropalladium(II) (Pd(dtbpf)Cl₂) (380 mg, 0.583 mmol, 0.2 equiv) and Cs₂CO₃ (1.91 g, 5.86 mmol, 2 equiv) at room temperature under nitrogen. The resulting mixture was stirred for 1 h at 80° C. then cooled to it and diluted with Water (100 mL). The solution was extracted with ethyl acetate (EtOAc) (3×200 mL), and the combined organic layers were washed with brine (150 mL), dried over anhydrous Na₂SO₄, filtered, and concentrated under reduced pressure. The resulting residue was purified by silica gel column chromatography (eluting with petroleum ether/ethyl acetate (PE/EtOAc) (2:3)) to afford methyl 4-[2-cyano-6-(methoxymethyl)phenyl]-5-(2-methoxyethoxy)-6-oxopyran-2-carboxylate (700 mg, 51% yield). LCMS (ES, m/z)=374.1 [M+H]+.

Steps 4-5: N-(5-(5-acetamido-1H-pyrazol-1-yl)-1,3,4-thiadiazol-2-yl)-4-(2-cyano-6-(methoxymethyl)phenyl)-3-(2-methoxyethoxy)-2-oxo-2H-pyran-6-carboxamide (Compound 39-Ac) was prepared according to Step 3-4 of Example 10 by hydrolysis of methyl 4-[2-cyano-6-(methoxymethyl)phenyl]-5-(2-methoxyethoxy)-6-oxopyran-2-carboxylate (instead of methyl 4-(2,6-dicyanophenyl)-3-(2-methoxyethoxy)-2-oxo-2H-pyran-6-carboxylate) to provide 4-(2-cyano-6-(methoxymethyl)phenyl)-3-(2-methoxyethoxy)-2-oxo-2H-pyran-6-carboxylic acid (the “aryl-pyrone reagent”) and coupling of this reagent with N-(1-(5-amino-1,3,4-thiadiazol-2-yl)-1H-pyrazol-5-yl)acetamide (product of Step 3, Example 5; “ADT amine reagent”). LCMS (ES, m/z)=565.8 [M+H]+.

Step 6: N-(5-(5-amino-1H-pyrazol-1-yl)-1,3,4-thiadiazol-2-yl)-4-(2-cyano-6-(methoxymethyl)phenyl)-3-(2-methoxyethoxy)-2-oxo-2H-pyran-6-carboxamide (Compound 39) was prepared by acidic deprotection of N-(5-(5-acetamido-1H-pyrazol-1-yl)-1,3,4-thiadiazol-2-yl)-4-(2-cyano-6-(methoxymethyl)phenyl)-3-(2-methoxyethoxy)-2-oxo-2H-pyran-6-carboxamide (Compound 39-Ac) following Step 5 of Example 10. LCMS (ES, m/z)=524.6 [M+H]+; ¹H NMR (400 MHz, DMSO-d6) δ 7.96 (d, J=7.2 Hz, 1H), 7.87 (d, J=7.6, 1H), 7.70 (dd. J=8.0, 1.2 Hz, 1H), 7.45 (d, J=2.0 Hz, 1H), 7.35 (s, 1H), 5.44 (d, J=2.0 Hz, 1H), 4.37 (s, 2H), 4.33-4.25 (m, 2H), 3.33-3.31 (m, 2H), 3.30 (s, 3H), 3.01 (s, 3H).

### Example 32: N-(5-(5-amino-1H-pyrazol-1-yl)-1,3,4-thiadiazol-2-yl)-4-(2,6-dimethoxyphenyl)-3-(3-hydroxypropoxy)-2-oxo-2H-pyran-6-carboxamide (Compound 40) and N-(5-(5-acetamido-1H-pyrazol-1-yl)-1,3,4-thiadiazol-2-yl)-4-(2,6-dimethoxyphenyl)-3-(3-hydroxypropoxy)-2-oxo-2H-pyran-6-carboxamide (Compound 40-Ac)

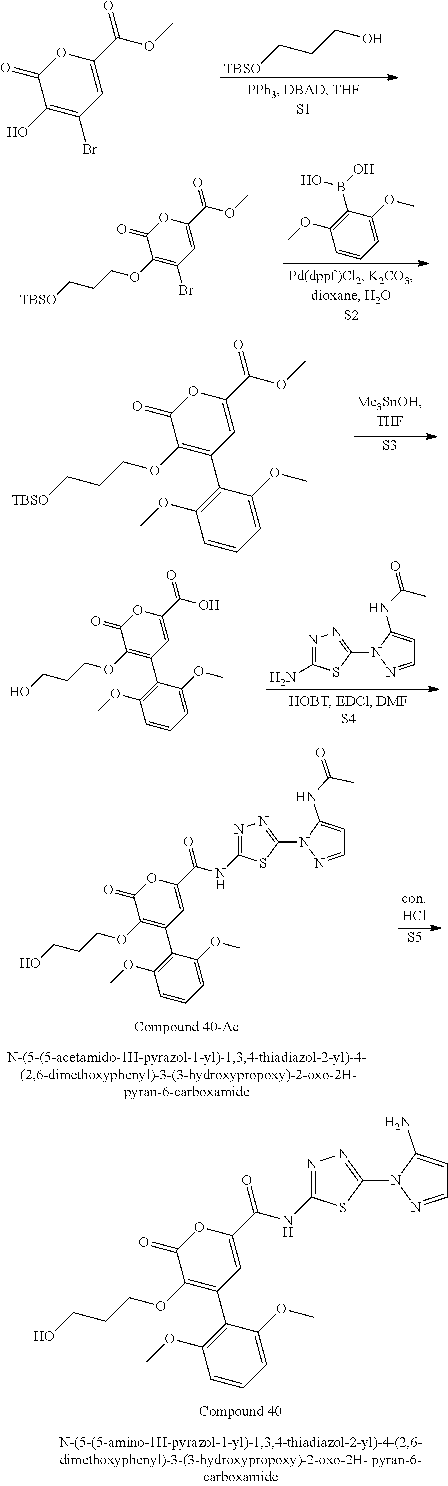

Step 1: To a stirred solution of methyl 4-bromo-5-hydroxy-6-oxopyran-2-carboxylate (product of Step 2, Example 1) (1 g, 4.0 mmol, 1 equiv) and 3-[(tert-butyldimethylsilyl)oxy]propan-1-ol (1 g, 5.2 mmol, 1.3 equiv) in tetrahydrofuran (THF) (4 mL) was added triphenyl phosphine (PPh₃) (1.05 g, 4.00 mmol, 1 equiv) and di-tert-butyl azodicarboxylate (DBAD) (0.93 g, 4.04 mmol, 1.01 equiv) in portions at 0° C. The resulting mixture was stirred for 2 h at room temperature then poured into water and extracted with ethyl acetate (EtOAc) (3×50 mL). The combined organic extracts were washed with brine (3×5 mL), dried over anhydrous Na₂SO₄, filtered, and concentrated under reduced pressure. The resulting residue was then purified by silica gel column chromatography (eluting with petroleum ether/ethyl acetate (PE/EtOAc) (12:1)) to afford methyl 4-bromo-5-{3-[(tert-butyldimethylsilyl)oxy]propoxy}-6-oxopyran-2-carboxylate (1 g, 59% yield). LCMS (ES, m/z)=421.2, 423.2 [M+H]+.

Step 2: methyl 3-(3-((tert-butyldimethylsilyl)oxy)propoxy)-4-(2,6-dinethoxyphenyl)-2-oxo-2H-pyran-6-carboxylate was prepared according to Step 1 of Example 29 using methyl 4-bromo-5-{3-[(tert-butyldimethylsilyl)oxy]propoxy}-6-oxopyran-2-carboxylate in place of methyl 4-bromo-5-{3-[(tert-butyldiphenylsilyl)oxy]-2,2-dimethylpropoxy}-6-oxopyran-2-carboxylate. LCMS (ES, m/z)=479.3 [M+H]+.

Step 3: To a solution of methyl 5-{3-[(tert-butyldimethylsilyl)oxy]propoxy}-4-(2,6-dimethoxyphenyl)-6-oxopyran-2-carboxylate (650 mg, 1.36 mmol, 1 equiv) in tetrahydrofuran (THF) (9 mL) was added trimethylstannanol (365 mg, 2.02 mmol, 1.49 equiv). The resulting solution was stirred overnight at room temperature then concentrated under reduced pressure. The residue was then purified by C18 reverse phase flash chromatography (conditions: column. C18; mobile phase, MeCN in Water, 10% to 50% gradient in 10 min: detector, UV 254 nm) to provide 4-(2,6-dimethoxyphenyl)-5-(3-hydroxypropoxy)-6-oxopyran-2-carboxylic acid (400 mg, 84% yield). LCMS (ES, m/z): 351.3 [M+H]+.

Step 4: N-(5-(5-acetamido-1H-pyrazol-1-yl)-1,3,4-thiadiazol-2-yl)-4-(2,6-dimethoxyphenyl)-3-(3-hydroxypropoxy)-2-oxo-2H-pyran-6-carboxamide (Compound 40-Ac) was prepared according to Step 1 of Example 8 using 4-(2,6-dimethoxyphenyl)-5-(3-hydroxypropoxy)-6-oxopyran-2-carboxylic acid (“aryl-pyrone reagent”) in place of 4-(3-cyanopyridin-2-yl)-5-(2-methoxyethoxy)-6-oxopyran-2-carboxylic acid and using N-(1-(5-amino-1,3,4-thiadiazol-2-yl)-1H-pyrazol-5-yl)acetamide (product of Step 3, Example 5; “ADT amine reagent”). LCMS (ES, m/z)=557.4 [M+H]+.

Step 5: N-(5-(5-amino-1H-pyrazol-1-yl)-1,3,4-thiadiazol-2-yl)-4-(2,6-dimethoxyphenyl)-3-(3-hydroxypropoxy)-2-oxo-2H-pyran-6-carboxamide (Compound 40) was prepared by acidic deprotection of N-(5-(5-acetamido-1H-pyrazol-1-yl)-1,3,4-thiadiazol-2-yl)-4-(2,6-dimethoxyphenyl)-3-(3-hydroxypropoxy)-2-oxo-2H-pyran-6-carboxamide (Compound 40-Ac) following Step 2 of Example 8. LCMS (ES, m/z)=515.2 [M+H]+; ¹H NMR (400 MHz, DMSO-d6) δ 13.20 (s, 1H), 7.48-7.40 (m, 2H), 7.27-7.24 (m, 1H), 6.80 (d, J=8.4 Hz, 2H), 6.66 (s, 2H), 5.45 (d, J=1.6 Hz, 1H), 4.35-4.30 (m, 1H), 4.11 (t, J=6.4 Hz, 2H), 3.77 (s, 6H), 3.32-3.27 (m, 2H), 1.62-1.55 (m, 2H).

### Example 33: (S)-3-((1,4-dioxan-2-yl)methoxy)-N-(5-(5-amino-1H-pyrazol-1-yl)-1,3,4-thiadiazol-2-yl)-4-(2,6-dimethoxyphenyl)-2-oxo-2H-pyran-6-carboxamide (Compound 43A*) and (R)-3-((1,4-dioxan-2-yl)methoxy)-N-(5-(5-amino-1H-pyrazol-1-yl)-1,3,4-thiadiazol-2-yl)-4-(2,6-dimethoxyphenyl)-2-oxo-2H-pyran-6-carboxamide (Compound 43B*), and 3-((1,4-dioxan-2-yl)methoxy)-N-(5-(5-acetamido-1H-pyrazol-1-yl)-1,3,4-thiadiazol-2-yl)-4-(2,6-dimethoxyphenyl)-2-oxo-2H-pyran-6-carboxamide (Compound 43-Ac)

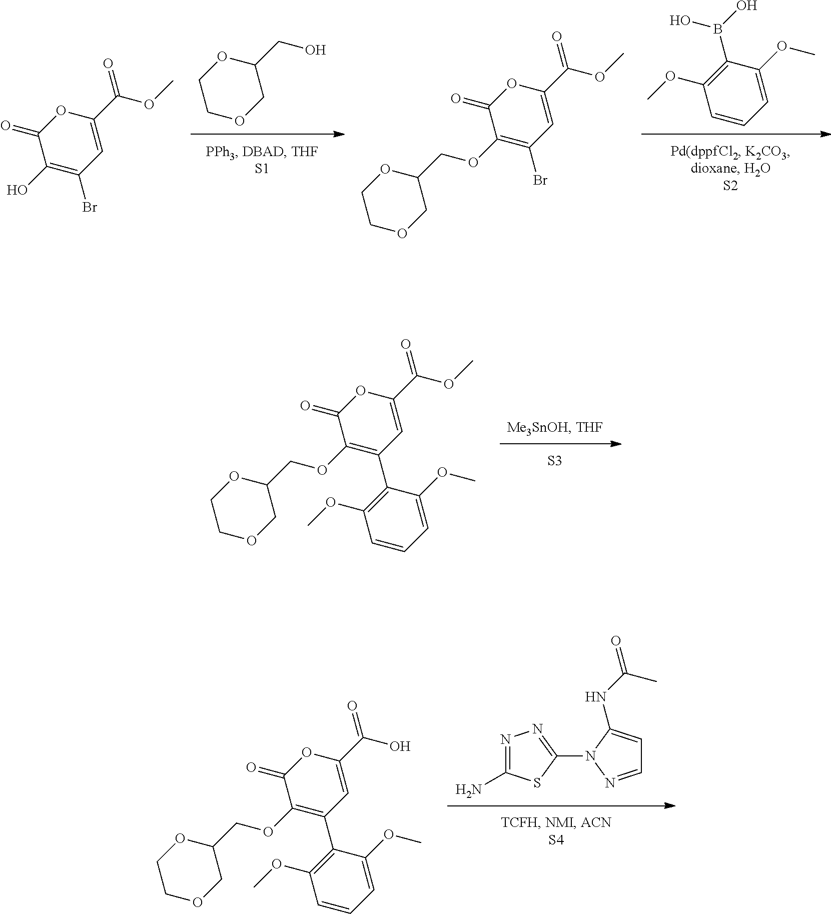

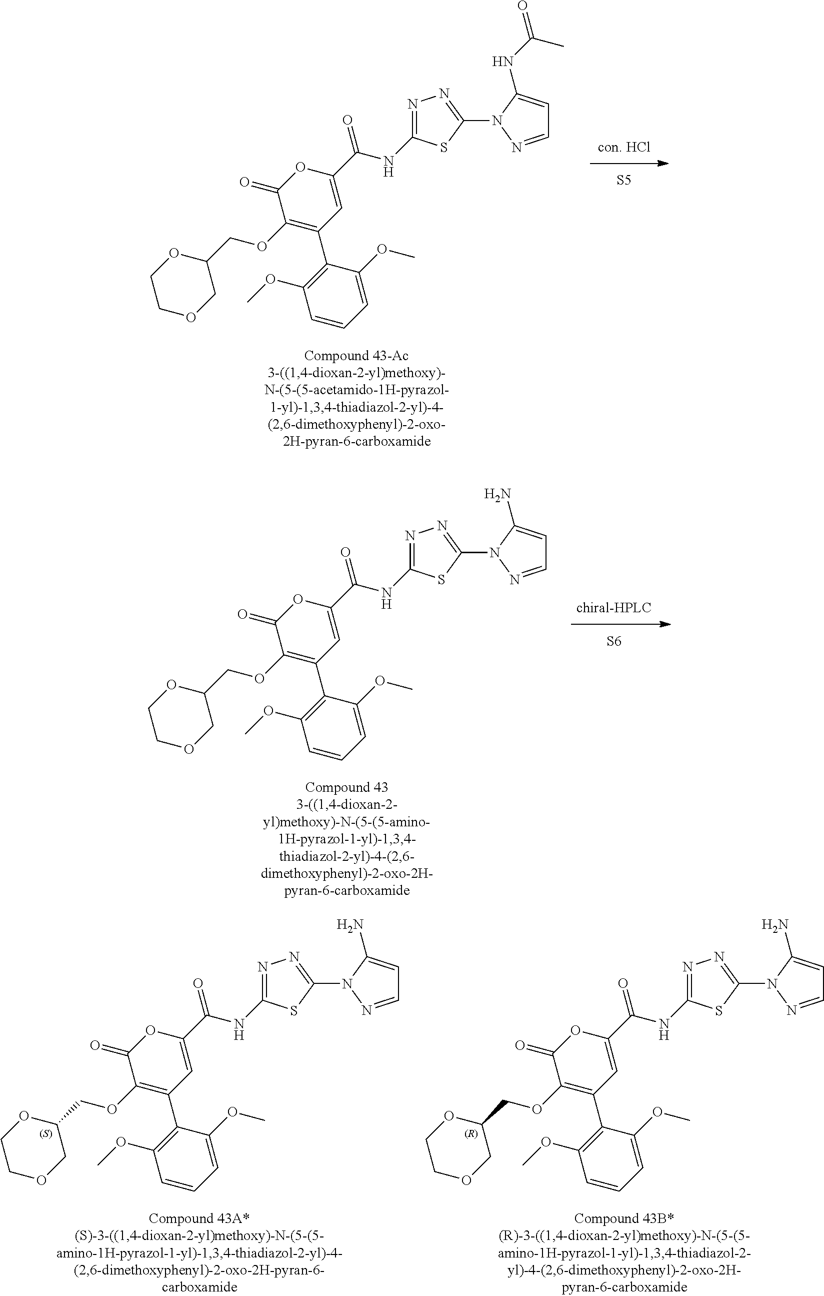

Step 1: Methyl 4-bromo-5-(1,4-dioxan-2-ylmethoxy)-6-oxopyran-2-carboxylate was prepared according to Step 1 of Example 3 using 1,4-dioxan-2-ylmethanol in place of 2-methoxyethanol. LCMS (ES, m/z)=349.0 [M+H]+.

Step 2: Methyl 4-(2,6-dimethoxyphenyl)-5-(1,4-dioxan-2-ylmethoxy)-6-oxopyran-2-carboxylate was prepared according to Step 2 of Example 18 using methyl 4-bromo-5-(1,4-dioxan-2-ylmethoxy)-6-oxopyran-2-carboxylate in place of methyl 4-bromo-5-[2-(morpholin-4-yl)ethoxy]-6-oxopyran-2-carboxylate. LCMS (ES, m/z)=407.2 [M+H]+.

Steps 3-4: 3-((1,4-dioxan-2-yl)methoxy)-N-(5-(5-acetamido-1H-pyrazol-1-yl)-1,3,4-thiadiazol-2-yl)-4-(2,6-dimethoxyphenyl)-2-oxo-2H-pyran-6-carboxamide (Compound 43-Ac) was prepared following Steps 3-4 of Example 16 by hydrolysis of methyl 4-(2,6-dimethoxyphenyl)-5-(1,4-dioxan-2-ylmethoxy)-6-oxopyran-2-carboxylate (instead of methyl 4-(2,6-dimethoxyphenyl)-5-[(1,3-dimethoxypropan-2-yl)oxy]-6-oxopyran-2-carboxylate) to provide 3-((1,4-dioxan-2-yl)methoxy)-4-(2,6-dimethoxyphenyl)-2-oxo-2H-pyran-6-carboxylic acid (“aryl-pyrone reagent”), followed by coupling of this reagent with N-(1-(5-amino-1,3,4-thiadiazol-2-yl)-1H-pyrazol-5-yl)acetamide (product of Step 3, Example 5; “ADT amine reagent”). LCMS (ES, m/z)=599.1 [M+H]+.

Steps 5-6:3-((1,4-dioxan-2-yl)methoxy)-N-(5-(5-amino-1H-pyrazol-1-yl)-1,3,4-thiadiazol-2-yl)-4-(2,6-dimethoxyphenyl)-2-oxo-2H-pyran-6-carboxamide (Compound 43) was prepared according to Step 5 of Example 16 by acidic deprotection of 3-((1,4-dioxan-2-yl)methoxy)-N-(5-(5-acetamido-1H-pyrazol-1-yl)-1,3,4-thiadiazol-2-yl)-4-(2,6-dimethoxyphenyl)-2-oxo-2H-pyran-6-carboxamide (Compound 43-Ac). The enantiomers of 3-((1,4-dioxan-2-yl)methoxy)-N-(5-(5-amino-1H-pyrazol-1-yl)-1,3,4-thiadiazol-2-yl)-4-(2,6-dimethoxyphenyl)-2-oxo-2H-pyran-6-carboxamide were separated by chiral prep-HPLC (CHIRALPAK IE, 2*25 cm; Mobile Phase A: Hexanes (0.1% TFA), Mobile Phase B: MeOH:DCM; Flow rate: 20 mL/min: Gradient: 50% B to 50% B in 28 min; Wave Length: 220/254 nm) to afford (S)-3-((1,4-dioxan-2-yl)methoxy)-N-(5-(5-amino-1H-pyrazol-1-yl)-1,3,4-thiadiazol-2-yl)-4-(2,6-dimethoxyphenyl)-2-oxo-2H-pyran-6-carboxamide (Compound 43A*, 8% yield) as the first eluting peak (chiral RT=20.01 min), and (R)-3-((1,4-dioxan-2-yl)methoxy)-N-(5-(5-amino-1H-pyrazol-1-yl)-1,3,4-thiadiazol-2-yl)-4-(2,6-dimethoxyphenyl)-2-oxo-2H-pyran-6-carboxamide (Compound 43B*, 15% yield) as the second eluting peak (chiral RT=24.67 min). Stereochemistry arbitrarily assigned.

Compound 43A*: LCMS (ES, m/z)=557.3 [M+H]+, ¹H NMR (400 MHz, DMSO-d6) δ 7.49-7.46 (m, 1H), 7.43 (d, J=2.0 Hz, 1H), 7.28 (s, 1H), 6.80 (d, J=8.4 Hz, 2H), 6.65 (s, 2H), 5.44 (d, J=2.0 Hz, 1H), 4.11-4.07 (m, 1H), 4.01-3.96 (m, 1H), 3.78 (d, J=1.2 Hz, 6H), 3.65-3.55 (m, 2H), 3.55-3.46 (m, 2H), 3.46-3.38 (m, 2H), 3.09-3.03 (m, 1H).

Compound 43B*: LCMS (ES, m/z)=557.2 [M+H]+, ¹H NMR (400 MHz, DMSO-d6) δ 7.51-7.40 (m, 2H), 7.30 (s, 1H), 6.80 (d, J=8.4 Hz, 2H), 6.66 (s, 2H), 5.45 (d, J=2.0 Hz, 1H), 4.11-4.08 (m, 1H), 4.00-3.96 (m, 1H), 3.78 (d, J=1.2 Hz, 6H), 3.66-3.56 (m, 2H), 3.55-3.48 (m, 2H), 3.44-3.40 (m, 2H), 3.09-3.04 (m, 1H).

### Example 34: N-(5-(5-amino-1H-pyrazol-1-yl)-1,3,4-thiadiazol-2-yl)-4-(2-chloro-6-cyanophenyl)-3-(2-methoxyethoxy)-2-oxo-2H-pyran-6-carboxamide (Compound 45), N-(5-(5-acetamido-1H-pyrazol-1-yl)-1,3,4-thiadiazol-2-yl)-4-(2-chloro-6-cyanophenyl)-3-(2-methoxyethoxy)-2-oxo-2H-pyran-6-carboxamide (Compound 45-Ac), N-(5-(5-acetamido-1H-pyrazol-1-yl)-1,3,4-thiadiazol-2-yl)-4-(2-carbamoyl-6-chlorophenyl)-3-(2-methoxyethoxy)-2-oxo-2H-pyran-6-carboxamide (Compound 25-Ac), and N-(5-(5-amino-1H-pyrazol-1-yl)-1,3,4-thiadiazol-2-yl)-4-(2-carbamoyl-6-chlorophenyl)-3-(2-methoxyethoxy)-2-oxo-2H-pyran-6-carboxamide (Compound 25)

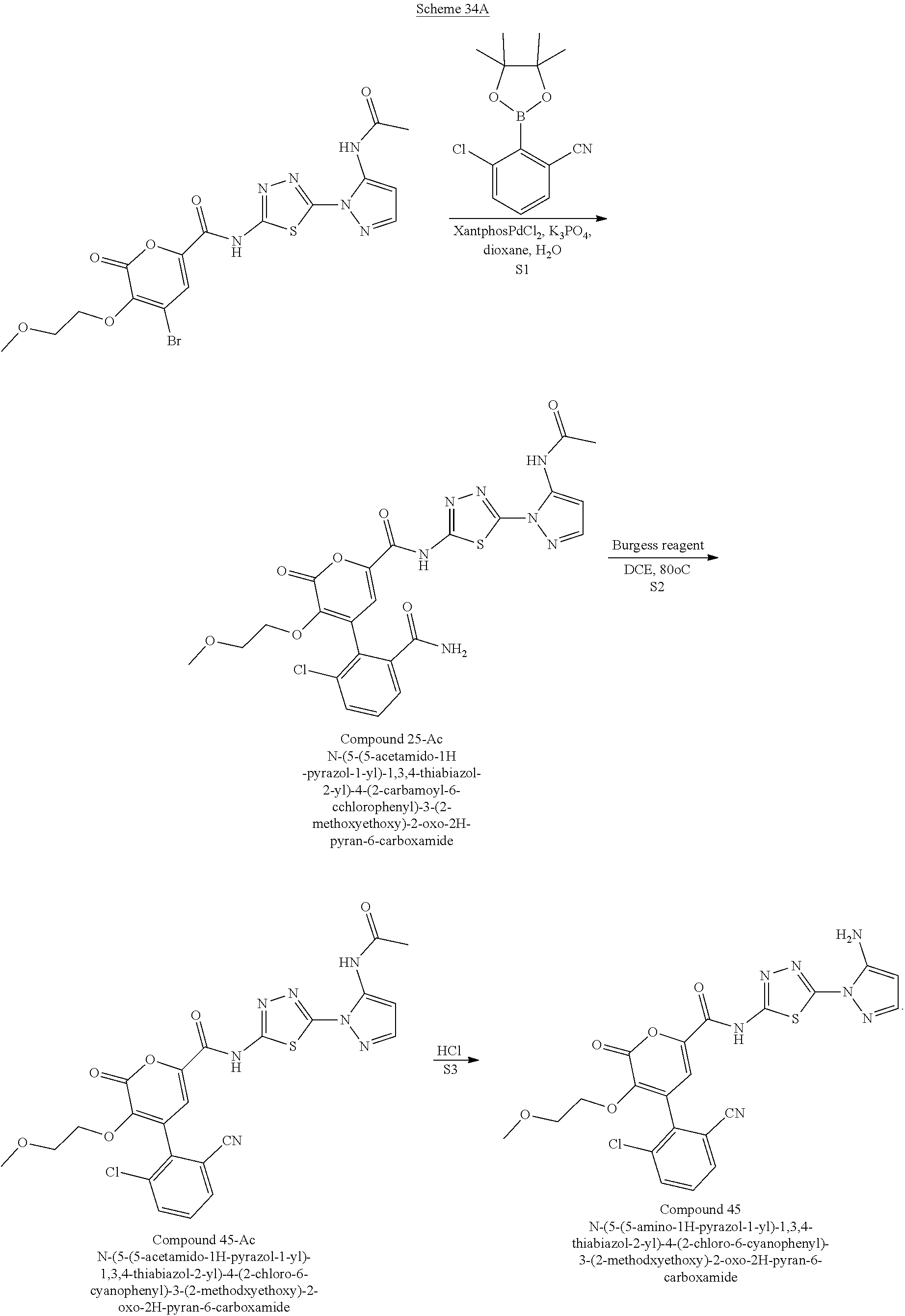

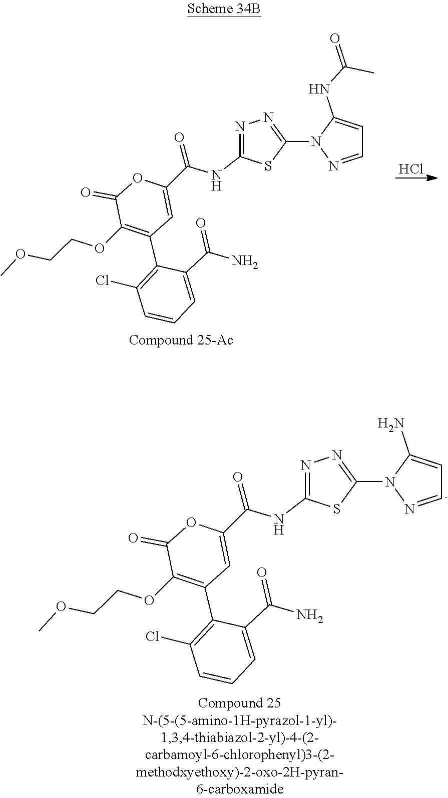

Step 1: To a solution of 4-bromo-N-[5-(5-acetamidopyrazol-1-yl)-1,3,4-thiadiazol-2-yl]-5-(2-methoxyethoxy)-6-oxopyran-2-carboxamide (product of Step 4, Example 5; “halo-pyrone reagent”) (200 mg, 0.401 mmol, 1 equiv) in dioxane (10 mL) was added 3-chloro-2-(4,4,5,5-tetramethyl-1,3,2-dioxaborolan-2-yl)benzonitrile (“aryl boron reagent”) (211 mg, 0.802 mmol, 2 equiv), dichloro[9,9-dimethyl-4,5-bis(diphenylphosphino)xanthene]palladium(II) (XantphosPdCl₂) (61 mg, 0.08 mmol, 0.2 equiv), K₃PO₄ (170 mg, 0.802 mmol, 2 equiv) and H₂O (1 mL) at room temperature. The resulting mixture was stirred overnight at 60° C. under nitrogen atmosphere then concentrated under reduced pressure. The resulting residue was purified by silica gel column chromatography (eluting with CH₂Cl₂/MeOH (10:1)) to afford N-(5-(5-acetamido-1H-pyrazol-1-yl)-1,3,4-thiadiazol-2-yl)-4-(2-carbamoyl-6-chlorophenyl)-3-(2-methoxyethoxy)-2-oxo-2H-pyran-6-carboxamide (Compound 25-Ac) (240 mg, 73% yield). LCMS (ES, m/z)=574.1 [M+H]+.

Step 2: To a solution of N-(5-(5-acetamido-1H-pyrazol-1-yl)-1,3,4-thiadiazol-2-yl)-4-(2-carbamoyl-6-chlorophenyl)-3-(2-methoxyethoxy)-2-oxo-2H-pyran-6-carboxamide (Compound 25-Ac) (250 mg, 0.436 mmol, 1 equiv) in dichloroethane (DCE) (5 mL) was added methyl N-(triethylammoniumsulfonyl) carbamate (Burgess reagent) (208 mg, 0.872 mmol, 2 equiv) at room temperature. The resulting mixture was stirred for 1 h at 80° C. then cooled to rt and diluted with water. The aqueous layer was extracted with CH₂Cl₂, dried over anhydrous Na₂SO₄, filtered, and concentrated under reduced pressure. The resulting residue was purified by C18 reverse phase flash chromatography (mobile phase, MeCN in water, 10% to 50% gradient in 10 min: detector, UV 254 nm) to provide N-(5-(5-acetamido-1H-pyrazol-1-yl)-1,3,4-thiadiazol-2-yl)-4-(2-chloro-6-cyanophenyl)-3-(2-methoxyethoxy)-2-oxo-2H-pyran-6-carboxamide (Compound 45-Ac) (100 mg, 39% yield). LCMS (ES, m/z)=554.1 [M−H]—.

Step 3: To a solution of N-(5-(5-acetamido-1H-pyrazol-1-yl)-1,3,4-thiadiazol-2-yl)-4-(2-chloro-6-cyanophenyl)-3-(2-methoxyethoxy)-2-oxo-2H-pyran-6-carboxamide (Compound 45-Ac) (50 mg, 0.090 mmol, 1 equiv) in ethanol (EtOH) (1.5 mL) was added conc. HCl (4.5 mL) at room temperature. The resulting mixture was stirred for 3 h at room temperature then concentrated under reduced pressure. The resulting residue was purified by C18 reverse phase flash chromatography (mobile phase, MeCN in water (0.1% TFA), 20% to 50% gradient in 20 min: detector, UV 254 nm) to provide N-(5-(5-amino-1H-pyrazol-1-yl)-1,3,4-thiadiazol-2-yl)-4-(2-chloro-6-cyanophenyl)-3-(2-methoxyethoxy)-2-oxo-2H-pyran-6-carboxamide (Compound 45) (21 mg, 37% yield). LCMS (ES, m/z)=514.1 [M+H]+; ¹H NMR (400 MHz, DMSO-d6) δ 13.42 (br, 1H), 8.05-8.01 (m, 2H), 7.75-7.71 (m, 1H), 7.56 (s, 1H), 7.48 (d, J=1.6 Hz, 1H), 6.65 (s, 2H), 5.45 (d, J=1.6 Hz, 1H), 4.46-4.31 (m, 2H), 3.38-3.36 (m, 2H), 3.04 (s, 3H).

N-(5-(5-amino-1H-pyrazol-1-yl)-1,3,4-thiadiazol-2-yl)-4-(2-carbamoyl-6-chlorophenyl)-3-(2-methoxyethoxy)-2-oxo-2H-pyran-6-carboxamide (Compound 25) may be prepared following above Step 3 of Example 34 by acidic deprotection of N-(5-(5-acetamido-1H-pyrazol-1-yl)-1,3,4-thiadiazol-2-yl)-4-(2-carbamoyl-6-chlorophenyl)-3-(2-methoxyethoxy)-2-oxo-2H-pyran-6-carboxamide (Compound 25-Ac).

### Example 35: (R)—N-(5-(5-amino-1H-pyrazol-1-yl)-1,3,4-thiadiazol-2-yl)-4-(2,6-dimethoxyphenyl)-3-(2-hydroxypropoxy)-2-oxo-2H-pyran-6-carboxamide (Compound 46A), (S)—N-(5-(5-amino-1H-pyrazol-1-yl)-1,3,4-thiadiazol-2-yl)-4-(2,6-dimethoxyphenyl)-3-(2-hydroxypropoxy)-2-oxo-2H-pyran-6-carboxamide (Compound 46B), (R)—N-(5-(5-acetamido-1H-pyrazol-1-yl)-1,3,4-thiadiazol-2-yl)-3-(2-(benzyloxy)propoxy)-4-(2,6-dimethoxyphenyl)-2-oxo-2H-pyran-6-carboxamide (Compound 46A-Ac-OBn), and (S)—N-(5-(5-acetamido-1H-pyrazol-1-yl)-1,3,4-thiadiazol-2-yl)-3-(2-(benzyloxy)propoxy)-4-(2,6-dimethoxyphenyl)-2-oxo-2H-pyran-6-carboxamide (Compound 46B-Ac-OBn)

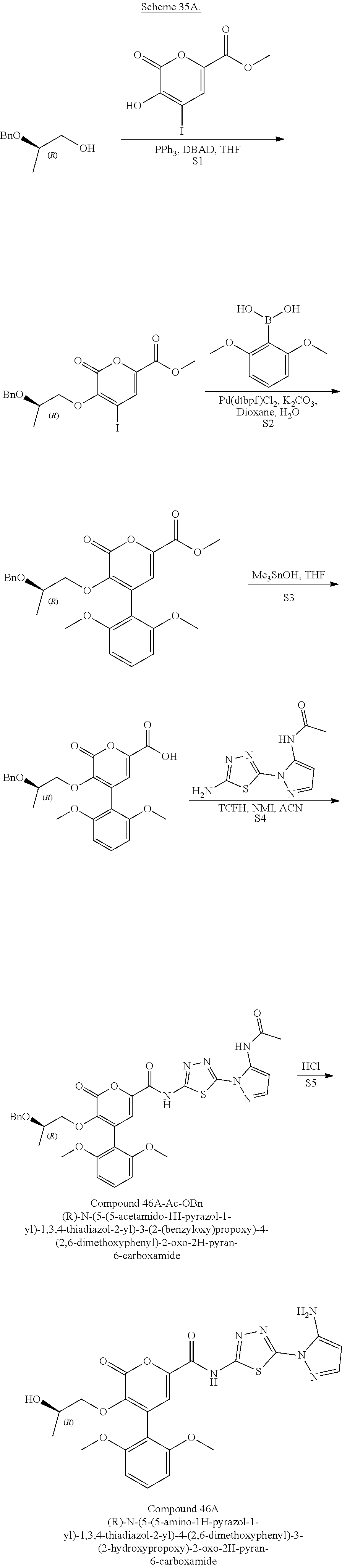

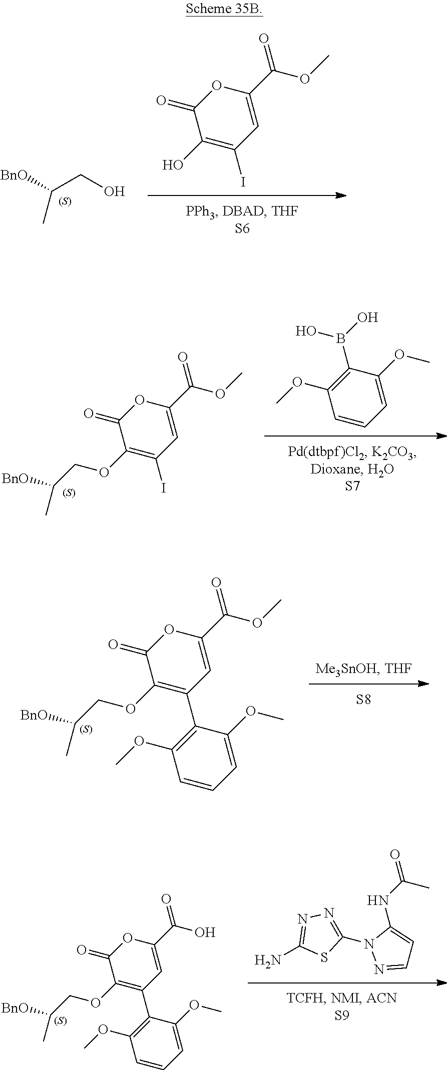

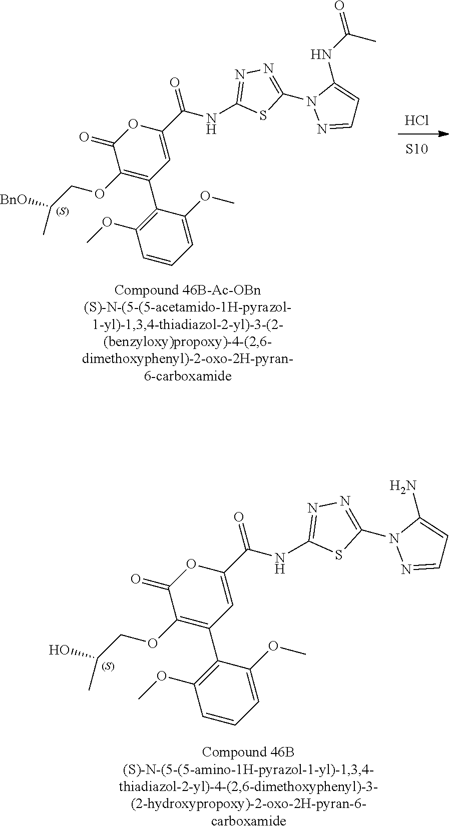

Step 1: Methyl 5-[(2R)-2-(benzyloxy)propoxy]-4-iodo-6-oxopyran-2-carboxylate was prepared according to Step 1 of Example 3 using (R)-2-(benzyloxy)propan-1-ol in place of 2-methoxyethanol and methyl 4-iodo-5-hydroxy-6-oxopyran-2-carboxylate in place of methyl 3-hydroxy-4-bromo-2-oxo-2H-pyran-6-carboxylate. LCMS (ES, m/z)=445.0 [M+H]+.

Step 2: Methyl 5-[(2R)-2-(benzyloxy)propoxy]-4-(2,6-dimethoxyphenyl)-6-oxopyran-2-carboxylate was prepared according to Step 2 of Example 18 using methyl 5-[(2R)-2-(benzyloxy)propoxy]-4-iodo-6-oxopyran-2-carboxylate in place of methyl 4-bromo-5-[2-(morpholin-4-yl)ethoxy]-6-oxopyran-2-carboxylate. LCMS (ES, m/z)=455.0 [M+H]+.

Steps 3-4: (R)—N-(5-(5-acetamido-1H-pyrazol-1-yl)-1,3,4-thiadiazol-2-yl)-3-(2-(benzyloxy)propoxy)-4-(2,6-dimethoxyphenyl)-2-oxo-2H-pyran-6-carboxamide (Compound 46A-Ac-OBn) was prepared following Steps 3-4 of Example 16 by hydrolysis of methyl 5-[(2R)-2-(benzyloxy)propoxy]-4-(2,6-dimethoxyphenyl)-6-oxopyran-2-carboxylate (instead of methyl 4-(2,6-dimethoxyphenyl)-5-[(1,3-dimethoxypropan-2-yl)oxy]-6-oxopyran-2-carboxylate) to provide (R)-3-(2-(benzyloxy)propoxy)-4-(2,6-dimethoxyphenyl)-2-oxo-2H-pyran-6-carboxylic acid (“aryl-pyrone reagent”), followed by coupling of this reagent with N-(1-(5-amino-1,3,4-thiadiazol-2-yl)-1H-pyrazol-5-yl)acetamide (product of Step 3, Example 5; “ADT amine reagent”).

Step 5: (R)—N-(5-(5-amino-1H-pyrazol-1-yl)-1,3,4-thiadiazol-2-yl)-4-(2,6-dimethoxyphenyl)-3-(2-hydroxypropoxy)-2-oxo-2H-pyran-6-carboxamide (Compound 46A) was prepared by acidic deprotection of (R)—N-(5-(5-acetamido-1H-pyrazol-1-yl)-1,3,4-thiadiazol-2-yl)-3-(2-(benzyloxy)propoxy)-4-(2,6-dimethoxyphenyl)-2-oxo-2H-pyran-6-carboxamide (Compound 46A-Ac-OBn) following Step 5 of Example 16. LCMS (ES, m/z)=515.2 [M+H]+, ¹H NMR (400 MHz, DMSO-d6) δ 7.41-7.35 (m, 1H), 7.33 (br, 1H), 6.86 (d, J=1.6 Hz, 1H), 6.76 (d, J=8.4 Hz, 2H), 6.61 (br, 2H), 5.39 (d, J=1.6 Hz, 1H), 3.99-3.94 (m, 1H), 3.75 (s, 6H), 3.65-3.52 (m, 2H), 0.86 (d, J=4.8 Hz, 3H).

Steps 6-10: (S)—N-(5-(5-amino-1H-pyrazol-1-yl)-1,3,4-thiadiazol-2-yl)-4-(2,6-dimethoxyphenyl)-3-(2-hydroxypropoxy)-2-oxo-2H-pyran-6-carboxamide (Compound 46B) was prepared following Steps 1-5 of above Example 35, but using (2S)-2-(benzyloxy) propan-1-ol in place of (2R)-2-(benzyloxy) propan-1-ol for Step 1. LCMS (ES, m/z)=515.1 [M+H]+. ¹H NMR (400 MHz, DMSO-d6) δ 13.21 (s, 1H), 7.51-7.38 (m, 2H), 7.27 (s, 1H), 6.83-6.75 (m, 1.7 Hz, 2H), 6.65 (s, 2H), 5.44 (t, J=1.8 Hz, 1H), 4.64-4.59 (m, 1H), 4.07-3.97 (m, 1H), 3.77 (t, J=2.2 Hz, 6H), 3.72-3.53 (m, 2H), 0.88-0.81 (m, 3H).

### Example 36: (S)—N-(5-(5-amino-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47A), (S)—N-(5-(5-acetamido-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47A-Ac), (R)—N-(5-(5-amino-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47B), (R)—N-(5-(5-acetamido-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47B-Ac)

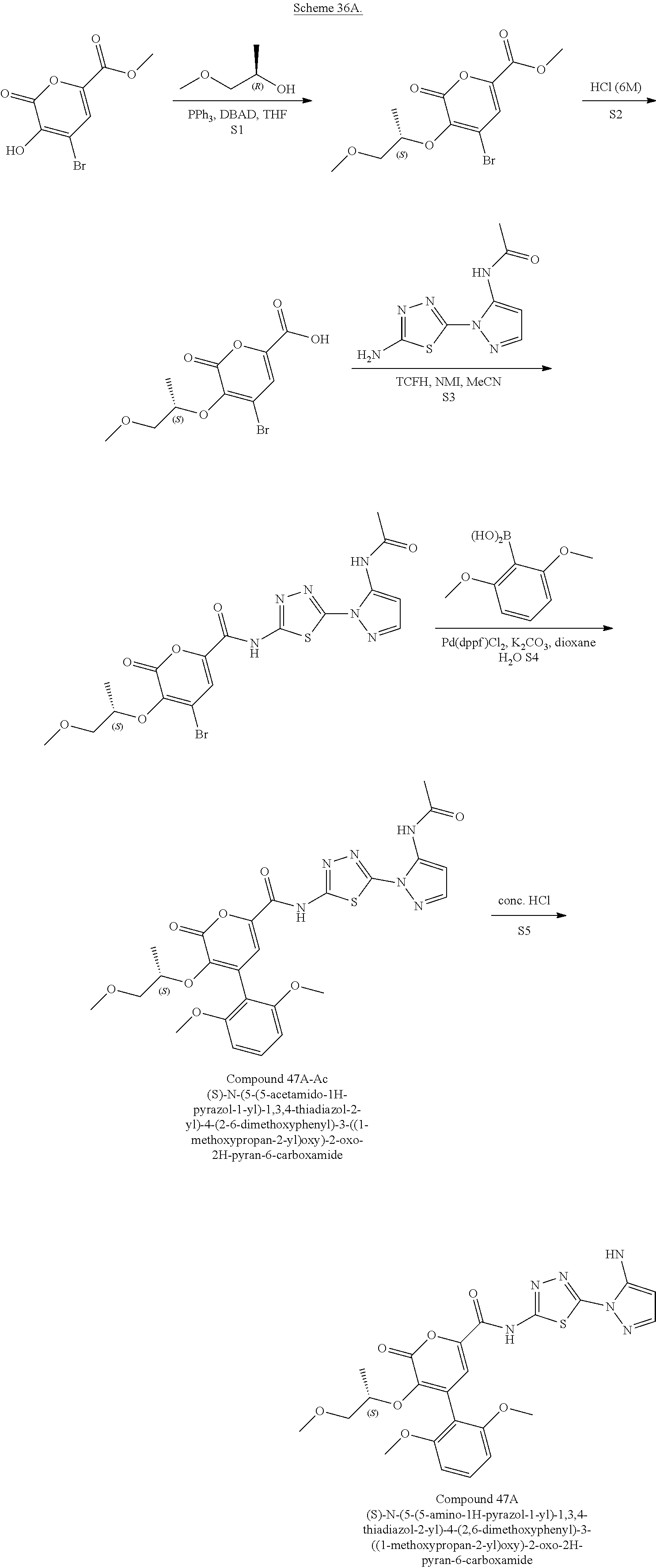

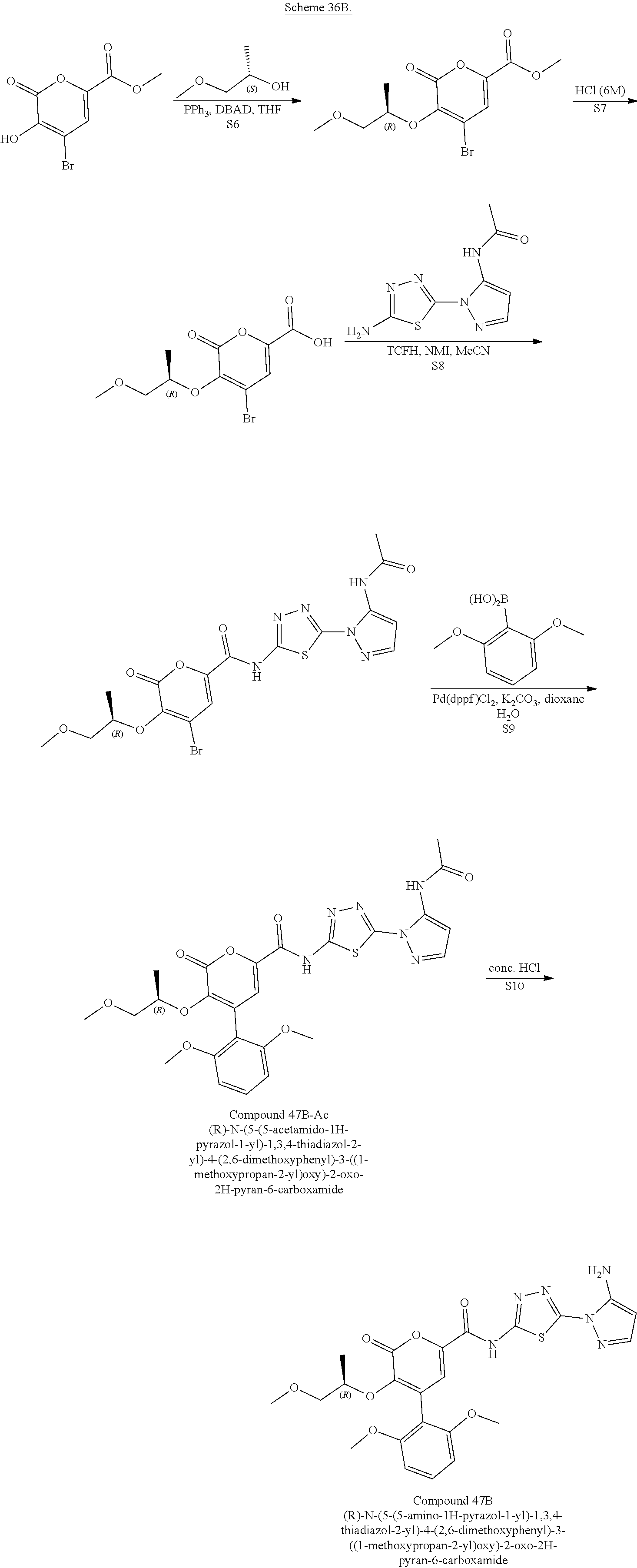

Step 1: To a stirred solution of methyl 4-bromo-5-hydroxy-6-oxopyran-2-carboxylate (product of Step 2, Example 1) (2 g, 8.0 mmol, 1 equiv) in tetrahydrofuran (THF) (20 mL) was added (2R)-1-methoxypropan-2-ol (1.45 g, 16.1 mmol, 2 equiv) and triphenyl phosphine (PPh₃) (6.36 g, 24.3 mmol, 3 equiv) at room temperature. To the above mixture was added di-tert-butyl azodicarboxylate (DBAD) (5.56 g, 24.2 mmol, 3 equiv) at 0° C. The resulting mixture was stirred for 4 h at room temperature then diluted with water (20 mL) and extracted with ethyl acetate (EtOAc) (3×50 mL). The combined organic layers were washed with brine (10 mL), dried over anhydrous Na₂SO₄, filtered, and concentrated under reduced pressure. The resulting residue was purified by silica gel column chromatography (eluting with petroleum ether/ethyl acetate (PE/EtOAc) (1:1)) to afford methyl 4-bromo-5-([(2S)-1-methoxypropan-2-yl]oxy-6-oxopyran-2-carboxylate (5.7 g, >100% yield). LCMS (ES, m/z)=321.0 [M+H]+.

Step 2: A solution of methyl 4-bromo-5-([(2S)-1-methoxypropan-2-yl]oxy-6-oxopyran-2-carboxylate (2.5 g, 7.8 mmol, 1 equiv) in hydrogen chloride (10 mL, 6M) was stirred for 1 h at 80° C. The resulting mixture was concentrated under reduced pressure and the residue was purified by C18 reverse phase flash chromatography (mobile phase, MeCN in Water, 10% to 50% gradient in 10 min; detector. UV 254 nm) to provide 4-bromo-5-([(2S)-1-methoxypropan-2-yl]oxy-6-oxopyran-2-carboxylic acid (260 mg, 9.8% yield). LCMS (ES, m/z)=307.0 [M+H]+.

Step 3: To a stirred solution of 4-bromo-5-([(2S)-1-methoxypropan-2-yl]oxy-6-oxopyran-2-carboxylic acid (300 mg, 0.977 mmol, 1 equiv) in acetonitrile (MeCN) (6 mL) was added chloro-N,N,N′,N′-tetramethylformamidinium hexafluorophosphate (TCFH) (420 mg, 1.50 mmol, 1.5 equiv), N-methylimidazole (NMI) (240 mg, 2.92 mmol, 3 equiv) and N-(1-(5-amino-1,3,4-thiadiazol-2-yl)-1H-pyrazol-5-yl)acetamide (product of Step 3, Example 5) (180 mg, 0.803 mmol, 0.8 equiv) at room temperature. The resulting mixture was stirred overnight at room temperature then diluted with water (20 mL) and extracted with ethyl acetate (EtOAc) (3×30 mL). The combined organic layers were washed with brine (10 mL), dried over anhydrous Na₂SO₄, filtered, and concentrated under reduced pressure. The resulting residue was purified by silica gel column chromatography (eluting with petroleum ether/ethyl acetate (PE/EtOAc) (1:1)) to afford 4-bromo-N-[5-(5-acetamidopyrazol-1-yl)-1,3,4-thiadiazol-2-yl]-5-([(2S)-1-methoxypropan-2-yl]oxy-6-oxopyran-2-carboxamide (100 mg, 20% yield). LCMS (ES, m/z)=513.0 [M+H]+.

Step 4: To a stirred solution of 4-bromo-N-[5-(5-acetamidopyrazol-1-yl)-1,3,4-thiadiazol-2-yl]-5-([(2S)-1-methoxypropan-2-yl]oxy-6-oxopyran-2-carboxamide (“halo-pyrone reagent”) (90 mg, 0.175 mmol, 1 equiv) and [1,1′-bis(di-tert-butylphosphino)ferrocene]dichloropalladium(II) (Pd(dtbpf)Cl₂) (36 mg, 0.055 mmol, 0.3 equiv) in dioxane (1.5 mL) and H₂O (0.3 mL) was added K₂CO₃ (73 mg, 0.528 mmol, 3 equiv) and 2,6-dimethoxyphenylboronic acid (“aryl boron reagent”) (64 mg, 0.352 mmol, 2 equiv) at room temperature. The resulting mixture was stirred for 1 h at 80° C. under nitrogen atmosphere then cooled to rt and diluted with water (20 mL). The solution was then extracted with ethyl acetate (EtOAc) (3×30 mL), and the combined organic layers were washed with brine (10 mL), dried over anhydrous Na₂SO₄, filtered, and concentrated under reduced pressure. The resulting residue was purified by C18 reverse phase flash chromatography (mobile phase, MeCN in water (0.05% TFA), 10% to 50% gradient in 10 min; detector, UV 254 nm) to provide (S)—N-(5-(5-acetamido-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47A-Ac) (40 mg, 40% yield). LCMS (ES, m/z)=571.1 [M+H]+.

Step 5: A solution of (S)—N-(5-(5-acetamido-1H-pyrazol-1-yl)-1,3,4-thiadiazol-2-yl)-4-(2,6-dimethoxyphenyl)-3-((I-methoxypropan-2-yl)oxy)-2-oxo-2H-pyran-6-carboxamide (Compound 47A-Ac) (20 mg, 0.035 mmol, 1 equiv) in conc. HCl (0.5 mL) was stirred for 2 h at room temperature. The mixture was then concentrated under reduced pressure and the residue was purified by C18 reverse phase flash chromatography (mobile phase, MeCN in water (0.1% TFA), 10% to 50% gradient in 10 min; detector, UV 254 nm) to provide (S)—N-(5-(5-amino-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47A) (2.2 mg, 12% yield). ¹H NMR (400 MHz, DMSO-d6) δ 13.15 (br, 1H), 7.49 (d, J=2.0 Hz, 1H), 7.43 (d, J=8.4 Hz, 1H), 7.28 (s, 1H), 6.80-6.78 (m, 2H), 6.66 (s, 1H), 5.45 (d, J=2.0 Hz, 1H), 4.62-4.53 (m, 1H), 3.77 (d, J=5.2 Hz, 6H), 3.28-3.11 (m, 2H), 3.08 (s, 3H), 0.85 (d, J=6.4 Hz, 3H).

Steps 6-10: (S)—N-(5-(5-amino-1H-pyrazol-1-yl)-1,3,4-thiadiazol-2-yl)-4-(2,6-dimethoxyphenyl)-3-((1-methoxypropan-2-yl)oxy)-2-oxo-2H-pyran-6-carboxamide (Compound 47B) was prepared following Steps 1-5 of above Example 36, but using (2S)-1-methoxypropan-2-ol in place of (2R)-1-methoxypropan-2-ol for Step 1. LCMS (ES, m/z)=515.1 [M+H]+. ¹H NMR (400 MHz, DMSO-d6) δ 13.22 (br. S, 1H), 7.46 (d, J=2.0 Hz, 1H), 7.41 (d, J=8.4 Hz, 1H), 7.24 (s, 1H), 6.80-6.78 (m, 2H), 6.65 (s, 1H), 5.44 (d, J=2.0 Hz, 1H), 4.62-4.53 (m, 1H), 3.77 (d, J=5.2 Hz, 6H), 3.27-3.12 (m, 2H), 3.08 (s, 3H), 0.98 (d, J=6.4 Hz, 3H).

### Example 37: N-(5-(5-amino-1H-pyrazol-1-yl)-1,3,4-thiadiazol-2-yl)-4-(2-cyano-6-(hydroxymethyl)phenyl)-3-(2-methoxyethoxy)-2-oxo-2H-pyran-6-carboxamide (Compound 48)

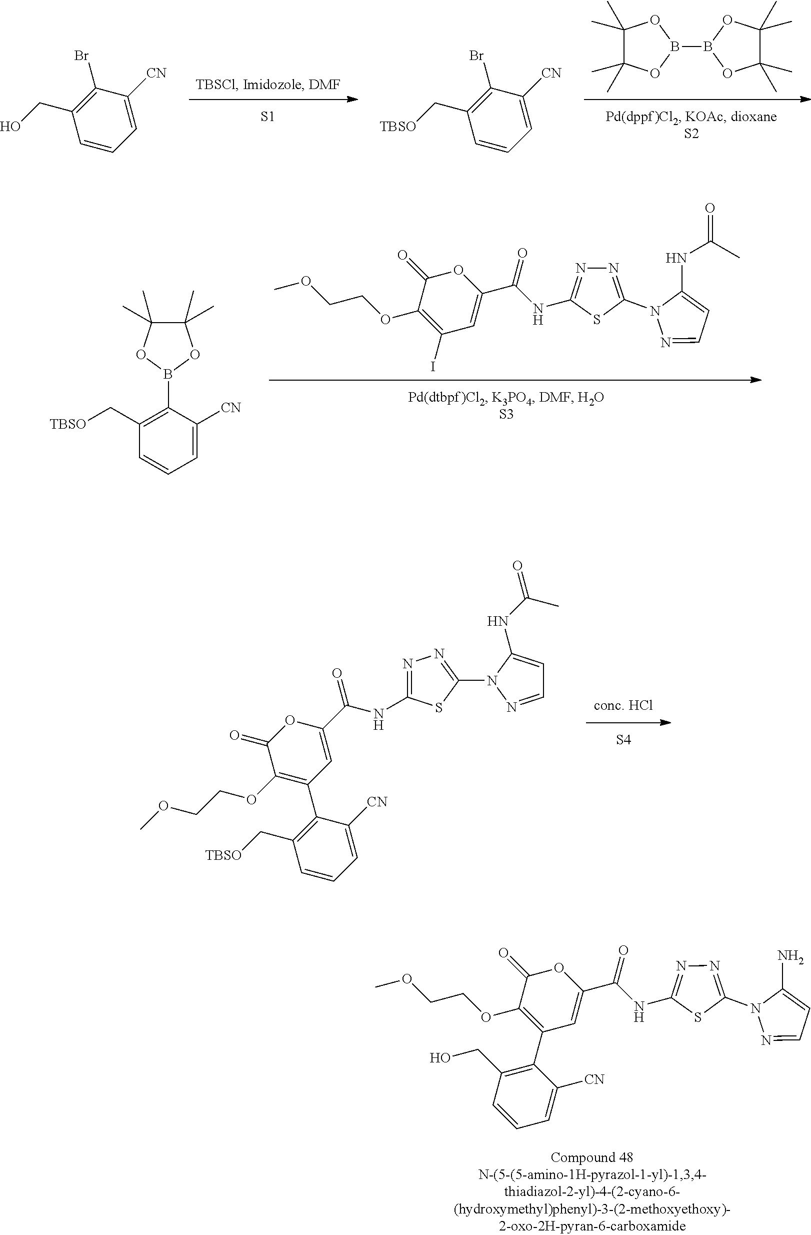

Step 1: To a solution of 2-bromo-3-(hydroxymethyl)benzonitrile (600 mg, 2.83 mmol, 1 equiv) in N,N-dimethylfonnamide (DMF) (5 mL) was added imidazole (577 mg, 8.48 mmol, 3 equiv) and tert-butyldimethylsilyl chloride (TBSCl) (849 mg, 5.63 mmol, 2 equiv) at room temperature. The resulting mixture was stirred for 16 h at room temperature then diluted with water and extracted with ethyl acetate (EtOAc) (3×50 mL). The organic layers were combined, washed with water (2×10 mL), then brine (10 mL), dried over anhydrous Na₂SO₄, filtered, and concentrated under reduced pressure. The resulting residue was purified by silica gel column chromatography (eluting with CH₂Cl₂/PE (1:9)) to afford 2-bromo-3-(((tert-butyldimethylsilyl)oxy)methyl)benzonitrile (900 mg, 78% yield). ¹H NMR (400 MHz, DMSO-d6) δ 7.90-7.84 (m, 1H), 7.84-7.78 (m, 1H), 7.71-7.61 (m, 1H), 4.75 (s, 2H), 0.93 (s, 9H), 0.13 (s, 6H).

Step 2: To a solution of 2-bromo-3-(((tert-butyldimethylsilyl)oxy)methyl)benzonitrile (900 mg, 2.76 mmol, 1 equiv) in dioxane (20 mL) was added bis(pinacolato)diboron (1057 mg, 4.165 mmol, 1.5 equiv), [1,1′-bis(diphenylphosphino)ferrocene]dichloro palladium(II) (Pd(dppf)Cl₂)—CH₂Cl₂ complex (540 mg, 0.663 mmol, 0.2 equiv) and potassium acetate (KOAc) (836 mg, 8.52 mmol, 3 equiv) at room temperature. The resulting mixture was stirred for 16 h at 100° C. under nitrogen atmosphere then cooled to rt and diluted with water. The mixture was extracted with ethyl acetate (EtOAc) (3×50 mL) and the organic layers were combined, dried over anhydrous Na₂SO₄, filtered, and concentrated under reduced pressure. The resulting residue was purified by silica gel column chromatography (eluting with CH₂Cl₂/PE (1:1)) to afford 3-([(tert-butyldimethylsilyl)oxy]methyl-2-(4,4,5,5-tetramethyl-1,3,2-dioxaborolan-2-yl)benzonitrile (500 mg, 39% yield). LCMS (ES, m/z)=374.2 [M+H]+.

Step 3: To a solution of 3-([(tert-butyldimethylsilyl)oxy]methyl-2-(4,4,5,5-tetramethyl-1,3,2-dioxaborolan-2-yl)benzonitrile (500 mg, 1.34 mmol, 1 equiv) in N,N-dimethylformamide (DMF) (20 mL) was added N-[5-(5-acetamidopyrazol-1-yl)-1,3,4-thiadiazol-2-yl]-4-iodo-5-(2-methoxyethoxy)-6-oxopyran-2-carboxamide (prepared following Steps 1-4 of Example 5, but using 4-iodo-3-(2-methoxyethoxy)-2-oxo-2H-pyran-6-carboxylic acid instead of 4-bromo-3-(2-methoxyethoxy)-2-oxo-2H-pyran-6-carboxylic acid) (300 mg, 0.549 mmol, 0.4 equiv), [1,1′-bis(di-tert-butylphosphino)ferrocene]dichloropalladium(II) (Pd(dtbpf)Cl₂) (168 mg, 0.258 mmol, 0.2 equiv), K₃PO₄ (543 mg, 2.56 mmol, 2 equiv) and H₂O (2 mL) at room temperature. The resulting mixture was stirred for 2 h at 100° C. under nitrogen atmosphere then cooled to rt and concentrated under reduced pressure. The crude residue was purified by silica gel column chromatography (eluting with CH₂Cl₂/MeOH (85:15)) followed by additional purification by C18 reverse phase flash chromatography (mobile phase, MeCN in water (0.1% TFA), 20% to 50% gradient in 20 min: detector, UV 254 nm) to provide 4-[2-cyano-6-(hydroxymethyl)phenyl]-N-[5-(5-acetamidopyrazol-1-yl)-1,3,4-thiadiazol-2-yl]-5-(2-methoxyethoxy)-6-oxopyran-2-carboxamide (150 mg, 15% yield). LCMS (ES, m/z)=666.2 [M+H]+.

Step 4: A solution of 4-[2-cyano-6-(hydroxymethyl)phenyl]-N-[5-(5-acetamidopyrazol-1-yl)-1,3,4-thiadiazol-2-yl]-5-(2-methoxyethoxy)-6-oxopyran-2-carboxamide (158 mg, 0.286 mmol, 1 equiv) in conc. HCl (10 mL) was stirred for 4 h at room temperature then concentrated under reduced pressure. The crude residue was purified by C18 reverse phase flash chromatography (mobile phase, MeCN in water (0.1% TFA), 20% to 50% gradient in 10 min: detector, UV 254 nm) to provide N-(5-(5-amino-1H-pyrazol-1-yl)-1,3,4-thiadiazol-2-yl)-4-(2-cyano-6-(hydroxymethyl)phenyl)-3-(2-methoxyethoxy)-2-oxo-2H-pyran-6-carboxamide (Compound 48) (19 mg, 13% yield). LCMS (ES, m/z)=510.1 [M+H]+; ¹H NMR (400 MHz, DMSO-d6) δ 13.38 (s, 1H), 7.95-7.88 (m, 2H), 7.73-7.66 (m, 1H), 7.51-7.43 (m, 2H), 6.65 (s, 2H), 5.45 (d, J=2.0 Hz, 2H), 4.57-4.40 (m, 2H), 4.39-4.23 (m, 2H), 3.34 (t, J=4.4 Hz, 2H), 3.03 (s, 3H).

### Example 38: N-(5-(5-amino-1H-pyrazol-1-yl)-1,3,4-thiadiazol-2-yl)-4-(2-cyano-6-(hydroxymethyl)phenyl)-3-(2-methoxyethoxy)-2-oxo-2H-pyran-6-carboxamide (Compound 50) and N-(5-(5-acetamido-H-pyrazol-1-yl)-1,3,4-thiadiazol-2-yl)-3-((1,3-bis(benzyloxy)propan-2-yl)oxy)-4-(2,6-dimethoxyphenyl)-2-oxo-2H-pyran-6-carboxamide (Compound 50-Ac-OBn)

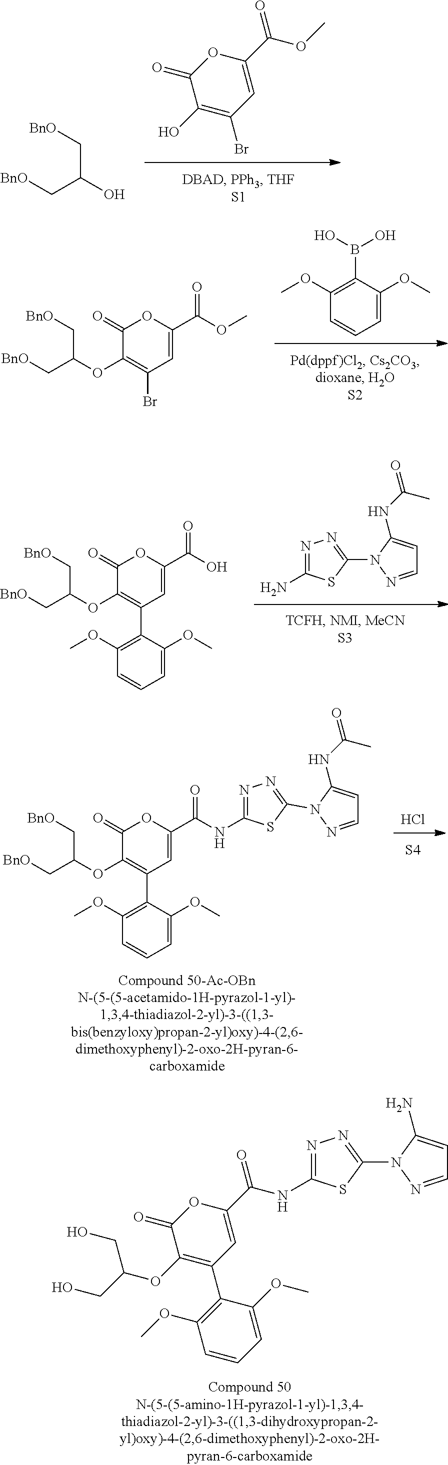

Step 1: Methyl 5-([1,3-bis(benzyloxy)propan-2-yl]oxy-4-bromo-6-oxopyran-2-carboxylate was prepared according to Step 1 of Example 3 using 1,3-bis(benzyloxy)propan-2-ol in place of 2-methoxyethanol. LCMS (ES, m/z)=503.0 [M+H]+.

Step 2: To a solution of methyl 5-([1,3-bis(benzyloxy)propan-2-yl]oxy-4-bromo-6-oxopyran-2-carboxylate (1.6 g, 3.2 mmol, 1 equiv) in dioxane (20 mL) and H₂O (4 mL) was added 2,6-dimethoxyphenylboronic acid (578 mg, 3.18 mmol, 1 equiv), [1,1′-bis(diphenylphosphino)ferrocene]dichloro palladium(II) (Pd(dppf)Cl₂)—CH₂Cl₂ complex (517 mg, 0.64 mmol, 0.2 equiv) and Cs₂CO₃ (2071 mg, 6.358 mmol, 2 equiv) at room temperature. The resulting mixture was stirred for 1.5 h at 100° C. under nitrogen atmosphere then cooled to rt, diluted with water, and acidified to pH 5 with HCl (1M). The resulting mixture was extracted with ethyl acetate (EtOAc) (3×30 mL), and the combined organic layers were dried over anhydrous Na₂SO₄, filtered, and concentrated under reduced pressure. The crude residue was purified by C18 reverse phase flash chromatography (mobile phase, MeCN in water, 50% to 100% gradient in 10 min; detector, UV 254 nm) to provide 5-([1,3-bis(benzyloxy)propan-2-yl]oxy-4-(2,6-dimethoxyphenyl)-6-oxopyran-2-carboxylic acid (500 mg, 26% yield). LCMS (ES, m/z)=547.1 [M+H]+.

Step 3: N-(5-(5-acetamido-1H-pyrazol-1-yl)-1,3,4-thiadiazol-2-yl)-3-((1,3-bis(benzyloxy)propan-2-yl)oxy)-4-(2,6-dimethoxyphenyl)-2-oxo-2H-pyran-6-carboxamide (Compound 50-Ac-OBn) was prepared according to Step 4 of Example 16 using 5-([1,3-bis(benzyloxy)propan-2-yl]oxy-4-(2,6-dimethoxyphenyl)-6-oxopyran-2-carboxylic acid (“aryl-pyrone reagent”) in place of 4-(2,6-dimethoxyphenyl)-5-[(1,3-dimethoxypropan-2-yl)oxy]-6-oxopyran-2-carboxylic acid and N-(1-(5-amino-1,3,4-thiadiazol-2-yl)-1H-pyrazol-5-yl)acetamide (product of Step 3, Example 5; “ADT amine reagent”).

Step 4: N-(5-(5-amino-1H-pyrazol-1-yl)-1,3,4-thiadiazol-2-yl)-4-(2-cyano-6-(hydroxymethyl)phenyl)-3-(2-methoxyethoxy)-2-oxo-2H-pyran-6-carboxamide (Compound 50) was prepared by acidic deprotection of N-(5-(5-acetamido-1H-pyrazol-1-yl)-1,3,4-thiadiazol-2-yl)-3-((1,3-bis(benzyloxy)propan-2-yl)oxy)-4-(2,6-dimethoxyphenyl)-2-oxo-2H-pyran-6-carboxamide (Compound 50-Ac-OBn) following Step 5 of Example 16. LCMS (ES, m/z)=531.1 [M+H]+: ¹H NMR (400 MHz, DMSO-d6) δ 13.19 (s, 1H), 7.48 (d, J=2.0 Hz, 1H), 7.42 (d, J=8.4 Hz, 1H), 7.27 (s, 1H), 6.78 (d, J=8.4 Hz, 2H), 6.68-6.63 (m, 2H), 5.45 (d, J=2.0 Hz, 1H), 4.45 (s, 2H), 4.35-4.32 (m, 1H), 3.77 (s, 6H), 3.43-3.35 (m, 2H), 3.34-3.30 (m, 2H).

### Example 39: N-(5-(5-aminothiazol-4-yl)-1,3,4-thiadiazol-2-yl)-4-(2,6-dimethoxyphenyl)-3-(2-methoxyethoxy)-2-oxo-2H-pyran-6-carboxamide (Compound 51) and N-(5-(5-acetamidothiazol-4-yl)-1,3,4-thiadiazol-2-yl)-4-(2,6-dimethoxyphenyl)-3-(2-methoxyethoxy)-2-oxo-2H-pyran-6-carboxamide (Compound 51-Ac)

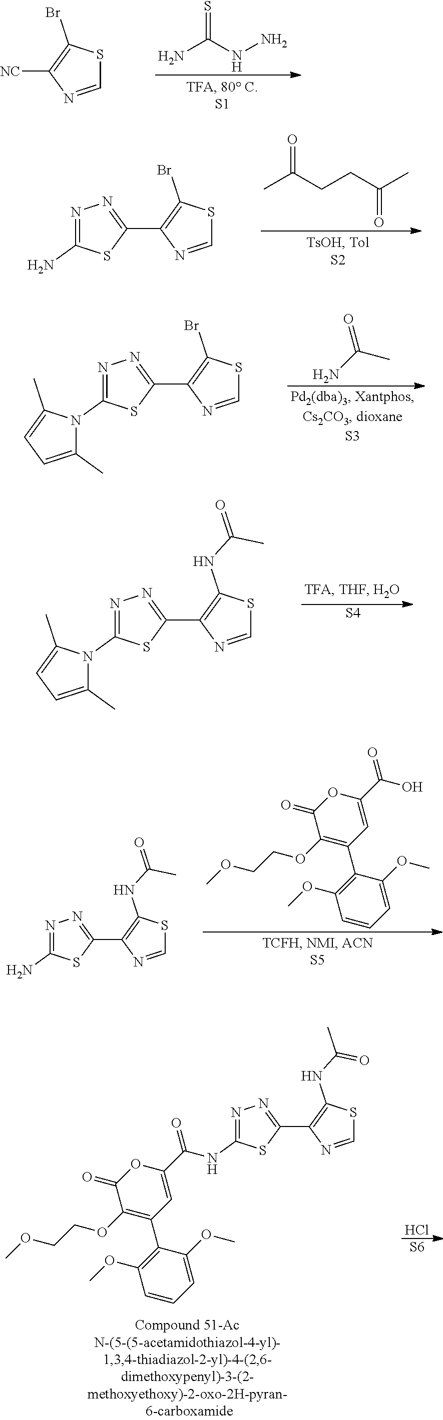

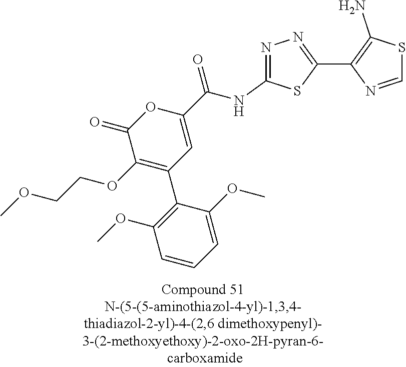 [SMILES: COCCOc1c(-c2c(OC)cccc2OC)cc(C(=O)Nc2nnc(-c3ncsc3N)s2)oc1=O]

Step 1: To a solution of 5-bromo-1,3-thiazole-4-carbonitrile (1 g, 5.3 mmol, 1 equiv) in trifluoroacetic acid (TFA) (20 mL) was added thiosemicarbazide (968 mg, 10.6 mmol, 2 equiv) at room temperature. The resulting mixture was stirred overnight at 80° C. then cooled to rt and concentrated under reduced pressure. The crude residue was purified by C18 reverse phase flash chromatography (mobile phase, MeCN in water, 10% to 50% gradient in 10 min: detector, UV 254 nm) to provide 5-(5-bromo-1,3-thiazol-4-yl)-1,34-thiadiazol-2-amine (900 mg, 58% yield). LCMS (ES, m/z)=263.0 [M+H]+.

Step 2: To a solution of 5-(5-bromo-1,3-thiazol-4-yl)-1,3,4-thiadiazol-2-amine (800 mg, 3 mmol, 1 equiv) in toluene (30 mL) was added 2,5-hexanedione (520 mg, 4.56 mmol, 1.5 equiv) and p-toluenesulfonic acid monohydrate (TsOH—H₂O) (116 mg, 0.61 mmol, 0.2 equiv) at room temperature. The resulting mixture was stirred for 2 h at 110° C. then cooled to rt and concentrated under reduced pressure. The crude residue was purified by silica gel column chromatography (eluting with petroleum ether/ethyl acetate (PE/EtOAc) (5:1)) to afford 2-(5-bromo-1,3-thiazol-4-yl)-5-(2,5-dimethylpyrrol-1-yl)-1,3,4-thiadiazole (500 mg, 46% yield). LCMS (ES, m/z)=341.0 [M+H]+.

Step 3: To a solution of 2-(5-bromo-1,3-thiazol-4-yl)-5-(2,5-dimethylpyrrol-1-yl)-1,3,4-thiadiazole (400 mg, 1.17 mmol, 1 equiv) in dioxane (8 mL) was added acetamide (346 mg, 5.86 mmol, 5 equiv), tris(dibenzylidenaceton)dipalladium(0) dibenzylidenacetone (Pd₂(dba)₃) (215 mg, 0.23 mmol, 0.20 equiv), (9,9-dimethyl-9H-xanthene-4,5-diyl)bis(diphenylphosphane) (XantPhos) (136 mg, 0.23 mmol, 0.2 equiv) and Cs₂CO₃ (764 mg, 2.34 mmol, 2 equiv) at room temperature. The resulting mixture was stirred for 1 h at 120° C. under nitrogen atmosphere then cooled to rt and concentrated under reduced pressure. The crude residue was purified by silica gel column chromatography (eluting with petroleum ether/ethyl acetate (PE/EtOAc) (5:1)) to afford N-(4-[5-(2,5-dimethylpyrrol-1-yl)-1,3,4-thiadiazol-2-yl]-1,3-thiazol-5-ylacetamide (300 mg, 72% yield). LCMS (ES, m/z)=318.1 [M−H]−.

Step 4: To a solution of N-(4-[5-(2,5-dimethylpyrrol-1-yl)-1,3,4-thiadiazol-2-yl]-1,3-thiazol-5-ylacetamide (300 mg, 0.94 mmol, 1 equiv) in tetrahydrofuran (THF) (5 mL) was added H₂O (1 mL) and trifluoroacetic acid (TFA) (5 mL) at room temperature. The resulting mixture was stirred for 2 h at 60° C. then cooled to rt and concentrated under reduced pressure. The crude residue was purified by C18 reverse phase flash chromatography (mobile phase, MeCN in water, 0% to 30% gradient in 10 min; detector, UV 254 nm) to provide N-[4-(5-amino-1,3,4-thiadiazol-2-yl)-1,3-thiazol-5-yl]acetamide (70 mg, 29% yield). LCMS (ES, m/z)=242.0 [M+H]+.

Step 5: To a solution of 4-(2,6-dimethoxyphenyl)-5-(2-methoxyethoxy)-6-oxopyran-2-carboxylic acid (“aryl-pyrone reagent”, prepared following Step 3-4 of Example 31, but using 2,6-dimethoxyphenylboronic acid instead of 3-(methoxymethyl)-2-(4,4,5,5-tetramethyl-1,3,2-dioxaborolan-2-yl)benzonitrile) (101 mg, 0.29 mmol, 1 equiv) in acetonitrile (MeCN) (2 mL) was added chloro-N,N,N′,N′-tetramethylformamidinium hexafluorophosphate (TCFH) (162 mg, 0.58 mmol, 2 equiv), N-methylimidazole (NMI) (47 mg, 0.58 mmol, 2 equiv) and N-[4-(5-amino-1,3,4-thiadiazol-2-yl)-1,3-thiazol-5-yl]acetamide (“ADT amine reagent”) (70 mg, 0.29 mmol, 1 equiv) at room temperature. The resulting mixture was stirred for 2 h at room temperature then concentrated under reduced pressure and the crude residue was purified by C18 reverse phase flash chromatography (mobile phase, MeCN in water (0.1% TFA), 10% to 70% gradient in 20 min; detector, UV 254 nm) to provide N-(5-(5-acetamidothiazol-4-yl)-1,3,4-thiadiazol-2-yl)-4-(2,6-dimethoxyphenyl)-3-(2-methoxyethoxy)-2-oxo-2H-pyran-6-carboxamide (Compound 51-Ac) (40 mg, 23% yield). LCMS (ES, m/z)=574.05 [M+H]+.

Step 6: A solution of N-(5-(5-acetamidothiazol-4-yl)-1,3,4-thiadiazol-2-yl)-4-(2,6-dimethoxyphenyl)-3-(2-methoxyethoxy)-2-oxo-2H-pyran-6-carboxamide (Compound 51-Ac) (30 mg, 0.052 mmol, 1 equiv) in conc. HCl (2 mL) was stirred for 1 h at 40° C. The mixture was then cooled to rt and concentrated under reduced pressure. The crude residue was purified by C18 reverse phase flash chromatography (mobile phase, MeCN in water (0.1% TFA), 10% to 50% gradient in 15 min; detector, UV 254 nm) to provide N-(5-(5-aminothiazol-4-yl)-1,3,4-thiadiazol-2-yl)-4-(2,6-dimethoxyphenyl)-3-(2-methoxyethoxy)-2-oxo-2H-pyran-6-carboxamide (Compound 51) (7.6 mg, 26% yield). LCMS (ES, m/z)=532.1 [M+H]+; ¹H NMR (400 MHz, DMSO-d6) δ 13.01 (br, 1H), 8.21 (s, 1H), 7.44-7.40 (m, 1H), 7.34-7.21 (m, 3H), 6.80-6.77 (m, 2H), 4.22-4.14 (m, 2H), 3.78 (s, 6H), 3.40-3.38 (m, 2H), 3.11 (s, 3H).

### Example 40: (S)—N-(5-(5-amino-1H-pyrazol-1-yl)-1,3,4-thiadiazol-2-yl)-4-(2,6-dimethoxyphenyl)-3-((1-hydroxypropan-2-yl)oxy)-2-oxo-2H-pyran-6-carboxamide (Compound 55A*), (R)—N-(5-(5-amino-1H-pyrazol-1-yl)-1,3,4-thiadiazol-2-yl)-4-(2,6-dimethoxyphenyl)-3-((1-hydroxypropan-2-yl)oxy)-2-oxo-2H-pyran-6-carboxamide (Compound 55B*), and N-(5-(5-acetamido-1H-pyrazol-1-yl)-1,3,4-thiadiazol-2-yl)-3-((1-(tert-butoxy)propan-2-yl)oxy)-4-(2,6-dimethoxyphenyl)-2-oxo-2H-pyran-6-carboxamide (Compound 55-Ac-OtBu)

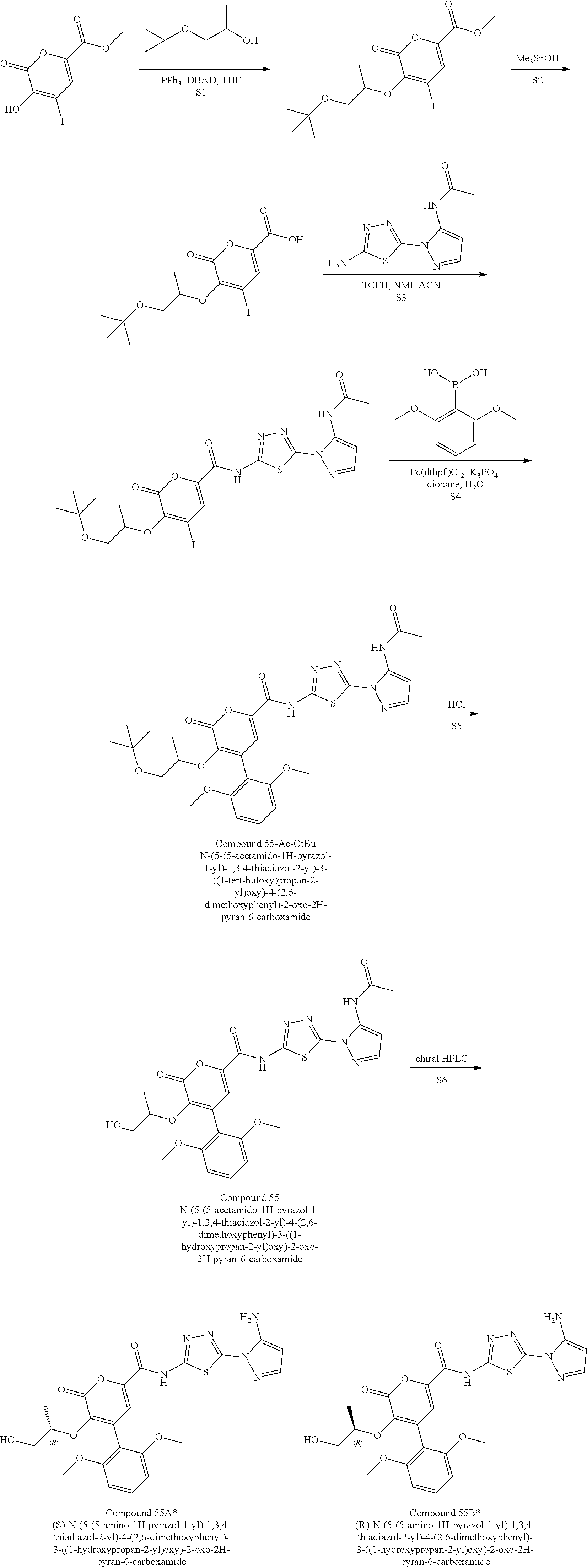

Steps 1-5: N-(5-(5-amino-1H-pyrazol-1-yl)-1,3,4-thiadiazol-2-yl)-4-(2,6-dimethoxyphenyl)-3-((1-hydroxypropan-2-yl)oxy)-2-oxo-2H-pyran-6-carboxamide (Compound 55) was prepared according to Steps 1-6 of Example 13 using propylene glycol t-butyl ether in place of 2-(t-butoxy)ethanol.

Step 6: The two enantiomers of N-(5-(5-amino-1H-pyrazol-1-yl)-1,3,4-thiadiazol-2-yl)-4-(2,6-dimethoxyphenyl)-3-((1-hydroxypropan-2-yl)oxy)-2-oxo-2H-pyran-6-carboxamide (Compound 55) was separated by chiral prep-HPLC (CHIRALPAK IE, 2*25 cm; Mobile Phase A: Hexanes (0.2% FA), Mobile Phase B: MeOH:DCM=1:1; Flow rate: 20 mL/min; Gradient: 50% B to 50% B in 33.2 min: Wave Length: 220/254 nm) to afford (S)—N-(5-(5-amino-1H-pyrazol-1-yl)-1,3,4-thiadiazol-2-yl)-4-(2,6-dimethoxyphenyl)-3-((1-hydroxypropan-2-yl)oxy)-2-oxo-2H-pyran-6-carboxamide (Compound 55A*) as the first eluting peak (chiral RT=25.12 min) and (R)—N-(5-(5-amino-1H-pyrazol-1-yl)-1,3,4-thiadiazol-2-yl)-4-(2,6-dimethoxyphenyl)-3-((1-hydroxypropan-2-yl)oxy)-2-oxo-2H-pyran-6-carboxamide (Compound 55B*) as the second eluting peak (chiral RT=30.22 min). Stereochemistry arbitrarily assigned.

Compound 55A*: LCMS (ES, m/z)=515.1 [M+H]+, ¹H NMR (400 MHz, DMSO-d6) δ 13.19 (s, 1H), 7.48 (d, J=2.0 Hz, 1H), 7.42-7.40 (m, 1H), 7.27 (s, 1H), 6.83-6.75 (m, 2H), 6.65 (s, 2H), 5.44 (d, J=2.0 Hz, 1H), 4.56 (br, 1H), 4.41-4.37 (m, 1H), 3.76 (s, 6H), 3.11-3.10 (m, 1H), 0.96 (d, J=6.4 Hz, 3H).

Compound 55B*: LCMS (ES, m/z)=515.1 [M+H]+, ¹H NMR (400 MHz, DMSO-d6) δ 13.19 (s, 1H), 7.48 (d, J=2.0 Hz, 1H), 7.42-7.40 (m, 1H), 7.27 (s, 1H), 6.83-6.75 (m, 2H), 6.65 (s, 2H), 5.44 (d, J=2.0 Hz, 1H), 4.56 (br, 1H), 4.41-4.37 (m, 1H), 3.76 (s, 6H), 3.11-3.10 (m, 1H), 0.96 (d, J=6.4 Hz, 3H).

### Example 41: N-(5-(5-amino-1H-pyrazol-1-yl)-1,3,4-thiadiazol-2-yl)-4-(2,6-dimethoxyphenyl)-3-(3-hydroxy-3-methylbutoxy)-2-oxo-2H-pyran-6-carboxamide (Compound 59) and N-(5-(5-acetamido-1H-pyrazol-1-yl)-1,3,4-thiadiazol-2-yl)-4-(2,6-dimethoxyphenyl)-3-(3-hydroxy-3-methylbutoxy)-2-oxo-2H-pyran-6-carboxamide (Compound 59-Ac)

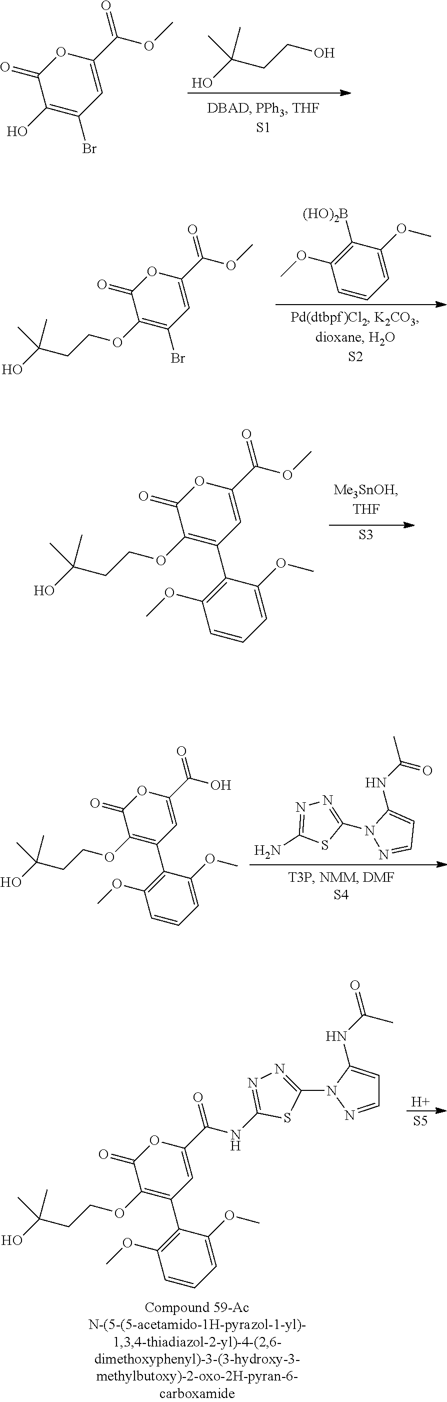

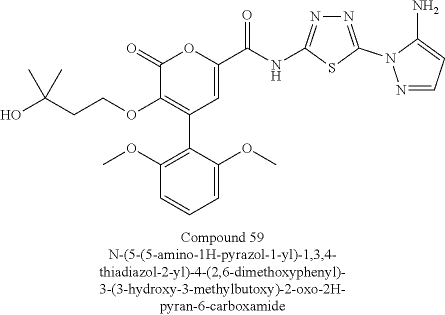 [SMILES: COc1cccc(OC)c1-c1cc(C(=O)Nc2nnc(-n3nccc3N)s2)oc(=O)c1OCCC(C)(C)O]

Steps 1-3: 4-(2,6-dimethoxyphenyl)-5-(3-hydroxy-3-methylbutoxy)-6-oxopyran-2-carboxylic acid was prepared according to Steps 1-3 of Example 32 using 2,4-dihydroxy-2-methylbutane in place of 3-[(tert-butyldimethylsilyl)oxy]propan-1-ol. LCMS (ES, m/z)=379.1 [M+H]+.

Step 4: To a stirred mixture of 4-(2,6-dimethoxyphenyl)-5-(3-hydroxy-3-methylbutoxy)-6-oxopyran-2-carboxylic acid (“aryl-pyrone reagent”) (60 mg, 0.16 mmol, 1 equiv) in N,N-dimethylformamide (DMF) (3.60 mL) was added a solution of propylphosphonic (T3P) in ethyl acetate (50% yield) (600 mg, 0.943 mmol, 6 equiv) and N-methylmorpholine (NMM) (96 mg, 0.95 mmol, 6 equiv) at room temperature. The resulting mixture was stirred for 0.5 h at room temperature, then N-(1-(5-amino-1,3,4-thiadiazol-2-yl)-1H-pyrazol-5-yl)acetamide (product of Step 3, Example 5; “ADT amine reagent”)(26 mg, 0.12 mmol, 0.7 equiv) was added and the resulting mixture was stirred for 1 h at room temperature then diluted with water (4 mL). The resulting solids were collected by filtration and washed with acetonitrile (3×4 mL) to provide N-(5-(5-acetamido-1H-pyrazol-1-yl)-1,3,4-thiadiazol-2-yl)-4-(2,6-dimethoxyphenyl)-3-(3-hydroxy-3-methylbutoxy)-2-oxo-2H-pyran-6-carboxamide (Compound 59-Ac) (25 mg, 2.4% yield). LCMS (ES, m/z)=585.1 [M+H]+.

Step 5: To a stirred mixture of N-(5-(5-acetamido-1H-pyrazol-1-yl)-1,3,4-thiadiazol-2-yl)-4-(2,6-dimethoxyphenyl)-3-(3-hydroxy-3-methylbutoxy)-2-oxo-2H-pyran-6-carboxamide (Compound 59-Ac) (20 mg, 0.034 mmol, 1 equiv) in MeOH (3.3 mL) was added acetyl chloride (0.55 mL) dropwise at room temperature. The resulting mixture was stirred for 1 h at room temperature then concentrated under reduced pressure. The crude residue was purified by C18 reverse phase flash chromatography (mobile phase. MeCN in water, 10% to 50% gradient in 10 min; detector, UV 254 nm) to provide N-(5-(5-amino-1H-pyrazol-1-yl)-1,3,4-thiadiazol-2-yl)-4-(2,6-dimethoxyphenyl)-3-(3-hydroxy-3-methylbutoxy)-2-oxo-2H-pyran-6-carboxamide (Compound 59) (9 mg, 48% yield). LCMS (ES, m/z)=543.2 [M+H]+; ¹H NMR (400 MHz, DMSO-d6) δ 13.23 (br, 1H), 7.53-7.37 (m, 2H), 7.25 (s, 1H), 6.79 (d, J=8.4 Hz, 2H), 6.66 (s, 2H), 5.44 (d, J=2.0 Hz, 1H), 4.22 (s, 1H), 4.15 (t, J=7.6 Hz, 2H), 3.77 (s, 6H), 1.55 (t, J=7.6 Hz, 2H), 0.98 (s, 6H).

### Example 42: N-(5-(5-amino-1H-pyrazol-1-yl)-1,3,4-thiadiazol-2-yl)-4-(3,5-dimethoxypyridin-2-yl)-3-(2-methoxyethoxy)-2-oxo-2H-pyran-6-carboxamide (Compound 60) and N-(5-(5-acetamido-1H-pyrazol-1-yl)-1,3,4-thiadiazol-2-yl)-4 (3,5-dimethoxypyridin-2-yl)-3-(2-methoxyethoxy)-2-oxo-2H-pyran-6-carboxamide (Compound 60-Ac)

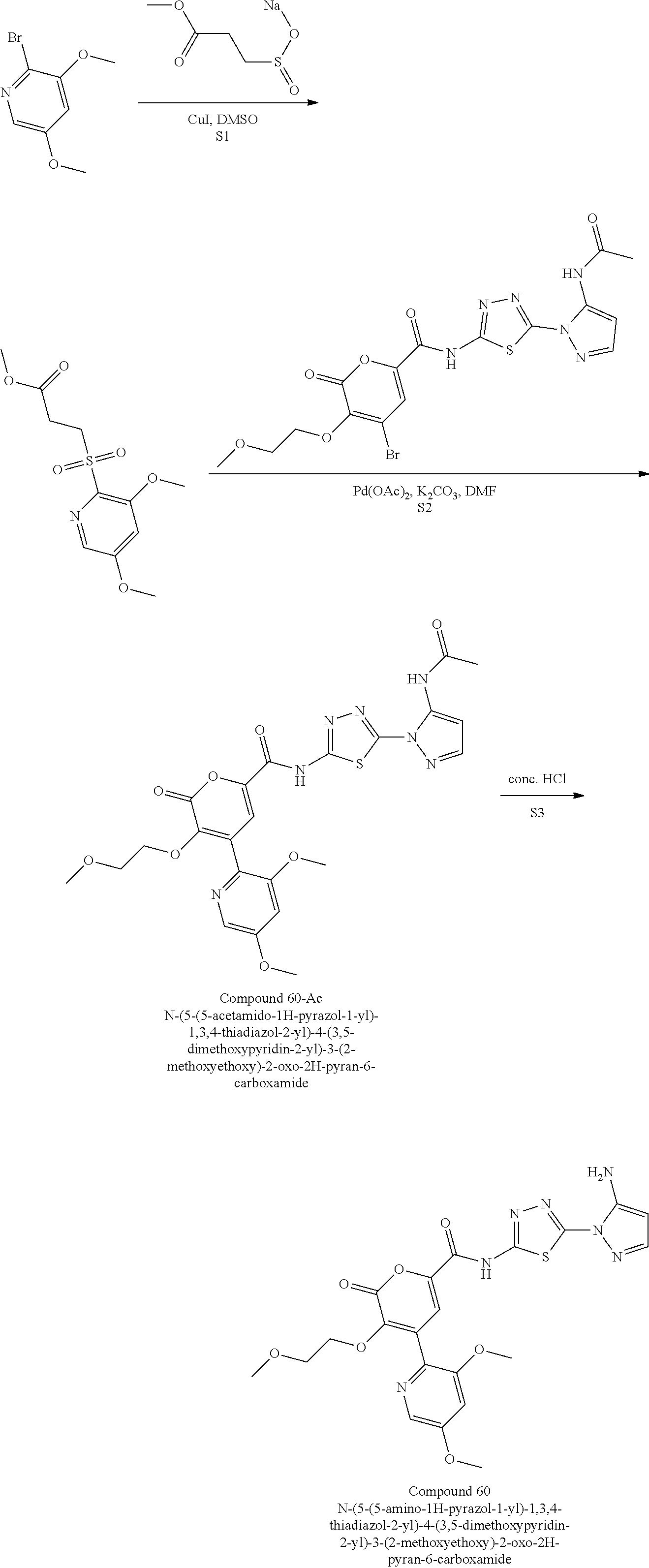

Steps 1-2: 4-(3,5-dimethoxypyridin-2-yl)-N-[5-(5-acetamidopyrazol-1-yl)-1,3,4-thiadiazol-2-yl]-5-(2-methoxyethoxy)-6-oxopyran-2-carboxamide was prepared according to Steps 1-2 of Example 6 using 2-bromo-3,5-dimethoxypyridine in place of 2-bromopyridine-3-carbonitrile and 4-bromo-N-[5-(5-acetamidopyrazol-1-yl)-1,3,4-thiadiazol-2-yl]-5-(2-methoxyethoxy)-6-oxopyran-2-carboxamide (product of Step 4, Example 5) in place of methyl 4-bromo-5-(2-methoxyethoxy)-6-oxopyran-2-carboxylate. LCMS (ESI, m/z)=558.1 [M+H]+.

Step 3: N-(5-(5-amino-1H-pyrazol-1-yl)-1,3,4-thiadiazol-2-yl)-4-(3,5-dimethoxypyridin-2-yl)-3-(2-methoxyethoxy)-2-oxo-2H-pyran-6-carboxamide (Compound 60) was prepared according to Step 2 of Example 7 by acidic deprotection of N-(5-(5-acetamido-1H-pyrazol-1-yl)-1,3,4-thiadiazol-2-yl)-4-(3,5-dimethoxypyridin-2-yl)-3-(2-methoxyethoxy)-2-oxo-2H-pyran-6-carboxamide (Compound 60-Ac). LCMS (ES, m/z)=516.6 [M+H]+; ¹H NMR (400 MHz, DMSO-d6) δ 13.29 (s, 1H), 8.06 (d, J=2.4 Hz, 1H), 7.48 (d, J=2.0 Hz, 2H), 7.22 (d, J=2.4 Hz, 1H), 6.65 (s, 2H), 5.45 (d, J=2.0 Hz, 1H), 4.24 (t, J=4.4 Hz, 2H), 3.94 (s, 3H), 3.88 (s, 3H), 3.45 (t, J=4.4 Hz, 2H), 3.13 (s, 3H).

### Example 43: (S)—N-(5-(5-amino-1H-pyrazol-1-yl)-1,3,4-thiadiazol-2-yl)-4-(2,6-dimethoxyphenyl)-3-((1-hydroxy-3-methoxypropan-2-yl)oxy)-2-oxo-2H-pyran-6-carboxamide (Compound 62A*), (R)—N-(5-(5-amino-1H-pyrazol-1-yl)-1,3,4-thiadiazol-2-yl)-4-(2,6-dimethoxyphenyl)-3-((1-hydroxy-3-methoxypropan-2-yl)oxy)-2-oxo-2H-pyran-6-carboxamide (Compound 62B*), (S)—N-(5-(5-amino-1H-pyrazol-1-yl)-1,3,4-thiadiazol-2-yl)-4-(2,6-dimethoxyphenyl)-3-(2-hydroxy-3-methoxypropoxy)-2-oxo-2H-pyran-6-carboxamide (Compound 63A*), and (R)—N-(5-(5-amino-1H-pyrazol-1-yl)-1,3,4-thiadiazol-2-yl)-4-(2,6-dimethoxyphenyl)-3-(2-hydroxy-3-methoxypropoxy)-2-oxo-2H-pyran-6-carboxamide (Compound 63B*)

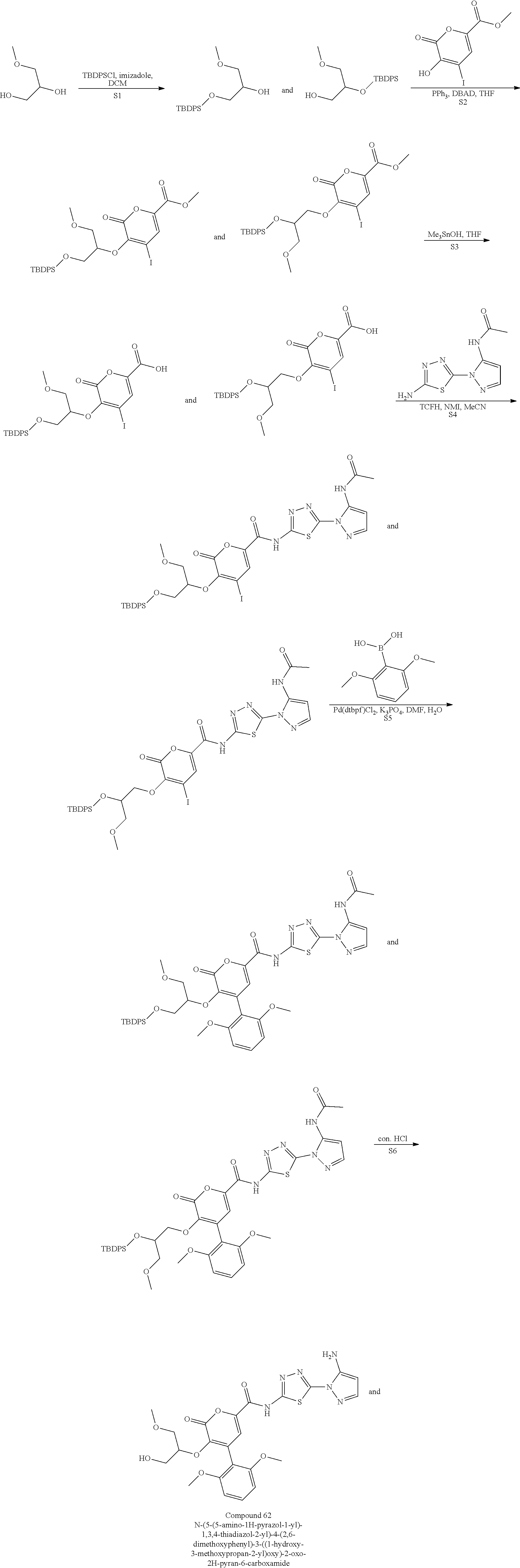

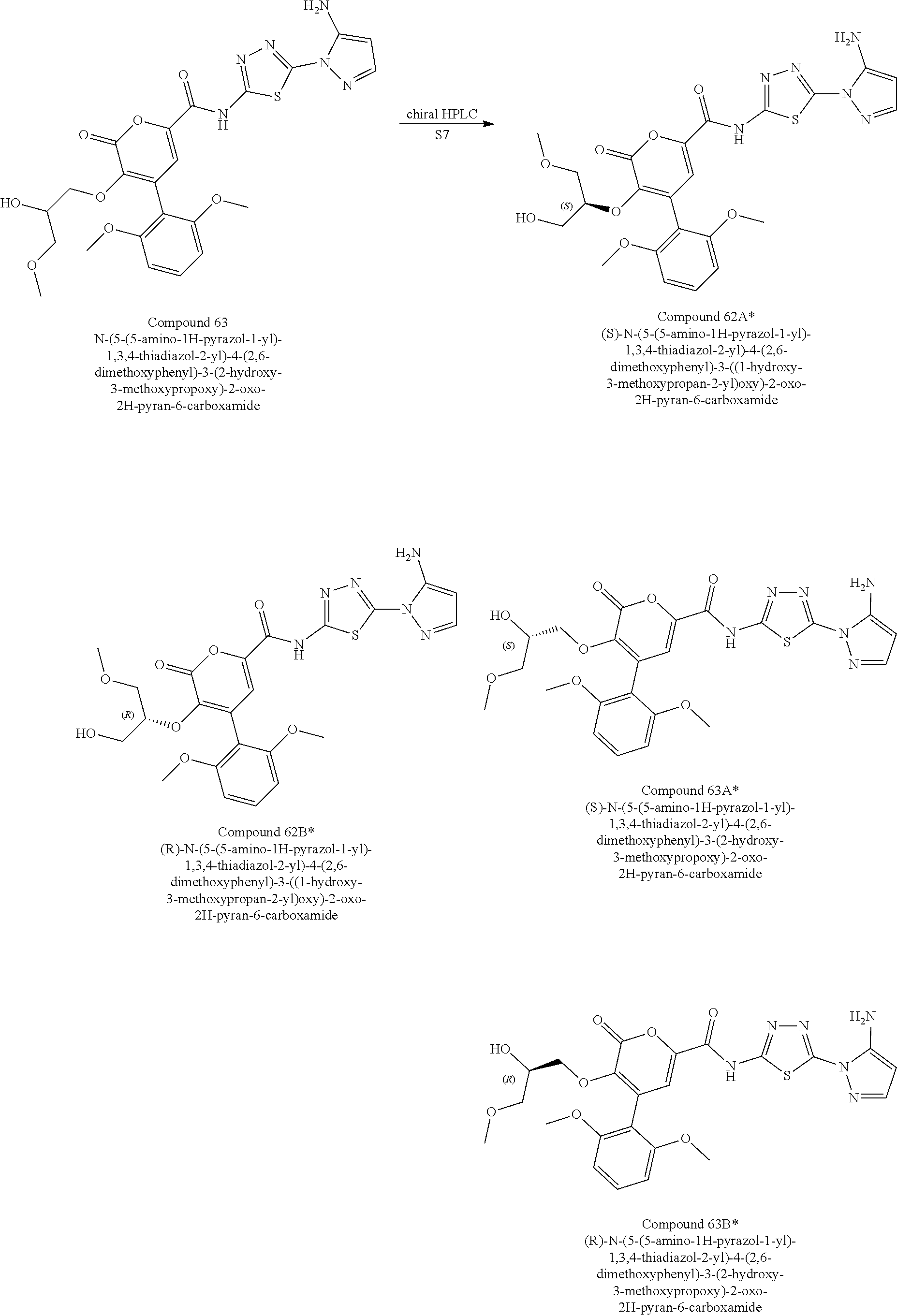

Step 1: To a solution of glycerin methyl ether (1 g, 9.4 mmol, 1 equiv) and 1H-imidazole (1.28 g, 18.8 mmol, 2 equiv) in dichloromethane (DCM) (20 mL) was added tert-butyl(chloro)diphenylsilane (2.85 g, 10.4 mmol, 1.1 equiv) dropwise at room temperature. The resulting mixture was stirred for 2 h at room temperature then diluted with water (50 mL) and extracted with dichloromethane (DCM) (3×50 mL). The combined organic layers were washed with brine (30 mL), dried over anhydrous Na₂SO₄, filtered, and concentrated under reduced pressure. The crude residue was purified by silica gel column chromatography (eluting with petroleum ether/ethyl acetate (PE/EtOAc) (8:1)) to afford a mixture of tert-butyl(2-hydroxy-3-methoxypropoxy)diphenylsilane and 2-((tert-butyldiphenylsilyl)oxy)-3-methoxypropan-1-ol (2.2 g, 68% yield). LCMS (ES, m/z)=345.1 [M+H]+.

Step 2: To a solution containing the mixture of tert-butyl(2-hydroxy-3-methoxypropoxy)diphenylsilane and 2-((tert-butyldiphenylsilyl)oxy)-3-methoxypropan-1-ol from Step 1 (2.2 g, 6.4 mmol, 1.2 equiv), triphenyl phosphine (PPh₃) (2.10 g, 7.98 mmol, 1.5 equiv) and methyl 5-hydroxy-4-iodo-6-oxopyran-2-carboxylate (product of Step 1, Example 11) (1.58 g, 5.34 mmol, 0.8 equiv) in tetrahydrofuran (THF) (25 mL) was added di-tert-butyl azodicarboxylate (DBAD) (1.84 g, 7.98 mmol, 1.5 equiv) dropwise at 0° C. under nitrogen atmosphere. The resulting mixture was stirred overnight at room temperature under nitrogen atmosphere then diluted with water (80 mL) and extracted with ethyl acetate (EtOAc) (3×200 mL). The combined organic layers were washed with brine (100 mL), dried over anhydrous Na₂SO₄, filtered, and concentrated under reduced pressure. The crude residue was purified by silica gel column chromatography (eluting with petroleum ether/ethyl acetate (PE/EtOAc) (10:1) to afford a mixture of methyl 5-({1-[(tert-butyldiphenylsilyl)oxy]-3-methoxypropan-2-yl)oxy}-4-iodo-6-oxopyran-2-carboxylate and methyl 3-(2-((tert-butyldiphenylsilyl)oxy)-3-methoxypropoxy)-4-iodo-2-oxo-2H-pyran-6-carboxylate (3 g, 90% yield). LCMS (ES, m/z)=623.1 [M+H]+.

Step 3: To a solution containing the mixture of methyl 5-({1-[(tert-butyldiphenylsilyl)oxy]-3-methoxypropan-2-yl}oxy)-4-iodo-6-oxopyran-2-carboxylate and methyl 3-(2-((tert-butyldiphenylsilyl)oxy)-3-methoxypropoxy)-4-iodo-2-oxo-2H-pyran-6-carboxylate from Step 2 (1.5 g, 2.4 mmol, 1 equiv) in tetrahydrofuran (THF) (15 mL) was added trimethylstannanol (872 mg, 4.82 mmol, 2 equiv). The resulting mixture was stirred for 2 h at room temperature then concentrated under reduced pressure. The crude residue was purified by C18 reverse phase flash chromatography (mobile phase. MeCN in water (0.05% TFA), 60% to 75% gradient in 15 min; detector, UV 254 nm) to provide a mixture of 5-({1-[(tert-butyldiphenylsilyl)oxy]-3-methoxypropan-2-yl)oxy}-4-iodo-6-oxopyran-2-carboxylic acid and 3-(2-((tert-butyldiphenylsilyl)oxy)-3-methoxypropoxy)-4-iodo-2-oxo-2H-pyran-6-carboxylic acid (1.1 g, 68% yield). LCMS (ES, m/z)=609.1 [M+H]+.

Step 4: To a solution containing the mixture of 5-({1-[(tert-butyldiphenylsilyl)oxy]-3-methoxypropan-2-yl}oxy)-4-iodo-6-oxopyran-2-carboxylic acid and 3-(2-((tert-butyldiphenylsilyl)oxy)-3-methoxypropoxy)-4-iodo-2-oxo-2H-pyran-6-carboxylic acid from Step 3 (1.5 g, 2.5 mmol, 1 equiv), chloro-N,N,N′,N′-tetramethylformamidinium hexafluorophosphate (TCFH) (2.10 g, 7.39 mmol, 3 equiv) and N-methylimidazole (NMI) (1.42 g, 17.2 mmol, 7 equiv) in acetonitrile (MeCN) (30 mL) was added N-(1-(5-amino-1,3,4-thiadiazol-2-yl)-1H-pyrazol-5-yl)acetamide (product of Step 3, Example 5; “ADT amine reagent”) (663 mg, 2.96 mmol, 1.2 equiv) at room temperature. The resulting mixture was stirred for 1 h at room temperature then diluted with water and extracted with ethyl acetate (EtOAc) (3×300 mL). The combined organic layers were washed with brine (100 mL), dried over anhydrous Na₂SO₄, filtered, and concentrated under reduced pressure. The crude residue was purified by C18 reverse phase flash chromatography (MeCN in water (50 mmol/L NH₄HCO₃), 45% to 65% gradient in 15 min: detector, UV 254 nm) to provide a mixture of 5-({1-[(tert-butyldiphenylsilyl)oxy]-3-methoxypropan-2-yl)oxy}-N-[5-(5-acetamidopyrazol-1-yl)-1,3,4-thiadiazol-2-yl]-4-iodo-6-oxopyran-2-carboxamide and N-(5-(5-acetamido-1H-pyrazol-1-yl)-1,3,4-thiadiazol-2-yl)-3-(2-((tert-butyldiphenylsilyl)oxy)-3-methoxypropoxy)-4-iodo-2-oxo-2H-pyran-6-carboxamide (400 mg, 18% yield). LCMS (ES, m/z)=815.3 [M+H]+.

Step 5: A solution containing the mixture of 5-({1-[(tert-butyldiphenylsilyl)oxy]-3-methoxypropan-2-yl}oxy)-N-[5-(5-acetamidopyrazol-1-yl)-1,3,4-thiadiazol-2-yl]-4-iodo-6-oxopyran-2-carboxamide and N-(5-(5-acetamido-1H-pyrazol-1-yl)-1,3,4-thiadiazol-2-yl)-3-(2-((tert-butyldiphenylsilyl)oxy)-3-methoxypropoxy)-4-iodo-2-oxo-2H-pyran-6-carboxamide from Step 4 (“halo-pyrone reagent”) (400 mg, 0.49 mmol, 1 equiv), 2,6-dimethoxyphenylboronic acid (“aryl boron reagent”) (134 mg, 0.74 mmol, 1.50 equiv), [1,1′-bis(di-tert-butylphosphino)ferrocene]dichloropalladium(II) (Pd(dtbpf)Cl₂) (64 mg, 0.098 mmol, 0.2 equiv) and K₃PO₄ (208 mg, 0.98 mmol, 2equiv) in N,N-dimethylformamide (DMF) (18 mL) and H₂O (3 mL) was stirred overnight at 80° C. The reaction mixture was then cooled to rt and directly purified by C18 reverse phase flash chromatography (mobile phase, MeCN in water (50 mmol/L NH₄HCO₃), 50% to 65% gradient in 15 min; detector, UV 254 nm) to provide a mixture of 5-({1-[(tert-butyldiphenylsilyl)oxy]-3-methoxypropan-2-yl}oxy)-4-(2,6-dimethoxyphenyl)-N-[5-(5-acetamidopyrazol-1-yl)-1,3,4-thiadiazol-2-yl]-6-oxopyran-2-carboxamide and N-(5-(5-acetamido-1H-pyrazol-1-yl)-1,3,4-thiadiazol-2-yl)-3-(2-((tert-butyldiphenylsilyl)oxy)-3-methoxypropoxy)-4-(2,6-dimethoxyphenyl)-2-oxo-2H-pyran-6-carboxamide (270 mg, 60% yield). LCMS (ES, m/z)=825.4 [M+H]+.

Steps 6-7: A solution containing the mixture of 5-((1-[(tert-butyldiphenylsilyl)oxy]-3-methoxypropan-2-yl)oxy)-4-(2,6-dimethoxyphenyl)-N-[5-(5-acetamidopyrazol-1-yl)-1,3,4-thiadiazol-2-yl]-6-oxopyran-2-carboxamide and N-(5-(5-acetamido-1H-pyrazol-1-yl)-1,3,4-thiadiazol-2-yl)-3-(2-((tert-butyldiphenylsilyl)oxy)-3-methoxypropoxy)-4-(2,6-dimethoxyphenyl)-2-oxo-2H-pyran-6-carboxamide from Step 5 (270 mg, 0.33 mmol, 1 equiv) in conc. HCl (4 mL) was stirred for 2 h at room temperature then concentrated under reduced pressure. The crude residue was purified by C18 reverse phase flash chromatography (mobile phase, MeCN in water (0.05% TFA), 30% to 50% gradient in 15 min: detector, UV 254 nm) followed by additional purification by Chiral-Prep-HPLC (CHIRAL ART Cellulose-SC, 2*25 cm; Mobile Phase A: Hexanes (0.2% FA), Mobile Phase B: EtOH:DCM=1:1; Flow rate: 20 mL/min; Gradient: 60% B to 60% B in 12 min; Wave Length: 220/254 nm) to afford (R)—N-(5-(5-amino-1H-pyrazol-1-yl)-1,3,4-thiadiazol-2-yl)-4-(2,6-dimethoxyphenyl)-3-((1-hydroxy-3-methoxypropan-2-yl)oxy)-2-oxo-2H-pyran-6-carboxamide (17.0 mg, 9% yield) (Compound 62B*) as the second eluting peak (chiral RT=10.39 min) and a mixture of (S)—N-(5-(5-amino-1H-pyrazol-1-yl)-1,3,4-thiadiazol-2-yl)-4-(2,6-dimethoxyphenyl)-3-((1-hydroxy-3-methoxypropan-2-yl)oxy)-2-oxo-2H-pyran-6-carboxamide (Compound 62A*), (S)—N-(5-(5-amino-1H-pyrazol-1-yl)-1,3,4-thiadiazol-2-yl)-4-(2,6-dimethoxyphenyl)-3-(2-hydroxy-3-methoxypropoxy)-2-oxo-2H-pyran-6-carboxamide (Compound 63A*), and (R)—N-(5-(5-amino-1H-pyrazol-1-yl)-1,3,4-thiadiazol-2-yl)-4-(2,6-dimethoxyphenyl)-3-(2-hydroxy-3-methoxypropoxy)-2-oxo-2H-pyran-6-carboxamide (Compound 63B*) which co-eluted as the first eluting peak (chiral RT=8.01). This mixture of isomers were then separated by additional chiral prep-HPLC (Lux Sum Cellulose-4, 2.12*25 cm: Mobile Phase A: Hexanes (0.1% TFA), Mobile Phase B: MeOH:EtOH=1:1; Flow rate: 20 mL/min: Gradient: 45% B to 45% B in 26 min; Wave Length: 220/254 nm) to afford (S)—N-(5-(5-amino-1H-pyrazol-1-yl)-1,3,4-thiadiazol-2-yl)-4-(2,6-dimethoxyphenyl)-3-(2-hydroxy-3-methoxypropoxy)-2-oxo-2H-pyran-6-carboxamide (3.3 mg, 2% yield) (Compound 63A*) as the first eluting peak (chiral RT=19.32 min), (R)—N-(5-(5-amino-1H-pyrazol-1-yl)-1,3,4-thiadiazol-2-yl)-4-(2,6-dimethoxyphenyl)-3-(2-hydroxy-3-methoxypropoxy)-2-oxo-2H-pyran-6-carboxamide (4.3 mg, 2% yield) (Compound 63B*) as the second eluting peak (chiral RT=21.32 min), and (S)—N-(5-(5-amino-1H-pyrazol-1-yl)-1,3,4-thiadiazol-2-yl)-4-(2,6-dimethoxyphenyl)-3-((1-hydroxy-3-methoxypropan-2-yl)oxy)-2-oxo-2H-pyran-6-carboxamide (10.7 mg, 6% yield) (Compound 62A*) as the third eluting peak (chiral RT=23.84 min). Stereochemistry arbitrarily assigned.

Compound 62A*: LCMS (ES, m/z)=543.1 [M−H]—. ¹H NMR (400 MHz, DMSO-d6) δ 7.48 (s, 1H), 7.42 (dd, J=8.4, 2.4 Hz, 1H), 7.26 (s, 1H), 6.79 (d, J=8.4 Hz, 2H), 5.46 (d, J=2.0 Hz, 1H), 4.46-4.43 (m, 1H), 3.76 (s, 6H), 3.53-3.17 (m, 5H), 3.06 (s, 3H).

Compound 62B*: LCMS (ES, m/z)=545.1 [M+H]+, ¹H NMR (400 MHz, DMSO-d6) δ 7.48 (s, 1H), 7.42 (dd, J=8.4, 2.4 Hz, 1H), 7.26 (s, 1H), 6.79 (d, J=8.4 Hz, 2H), 5.46 (d, J=2.0 Hz, 1H), 4.46-4.43 (m, 1H), 3.76 (s, 6H), 3.53-3.17 (m, 5H), 3.06 (s, 3H).

Compound 63A*: LCMS (ES, m/z)=545.2 [M+H]+, ¹H NMR (400 MHz, DMSO-d6) δ 7.48 (s, 1H), 7.43 (dd, J=8.4, 2.4 Hz, 1H), 7.26 (s, 1H); 6.80 (d, J=8.4 Hz, 2H), 5.46 (d, J=2.0 Hz, 1H), 4.03 (dd, J=9.6, 5.6 Hz, 1H), 3.91 (dd, J=9.6, 5.6 Hz, 1H), 3.76 (s, 6H), 3.60-3.58 (m, 1H), 3.10 (s, 3H), 3.07-3.03 (m, 2H).

Compound 63B*: LCMS (ES, m/z)=543.1 [M−H]−, ¹H NMR (400 MHz, DMSO-d6) δ 7.48 (s, 1H), 7.43 (dd, J=8.4, 2.4 Hz, 1H), 7.26 (s, 1H); 6.80 (d, J=8.4 Hz, 2H), 5.46 (d, J=2.0 Hz, 1H), 4.03 (dd, J=9.6, 5.6 Hz, 1H), 3.91 (dd, J=9.6, 5.6 Hz, 1H), 3.76 (s, 6H), 3.60-3.58 (m, 1H), 3.10 (s, 3H), 3.07-3.03 (m, 2H);

### Example 44: N-(5-(5-amino-1H-pyrazol-1-yl)-1,3,4-thiadiazol-2-yl)-4-(2-(2-cyanoethoxy)-6-methoxyphenyl)-3-(2-methoxyethoxy)-2-oxo-2H-pyran-6-carboxamide (Compound 67), N-(5-(5-acetamido-1H-pyrazol-1-yl)-1,3,4-thiadiazol-2-yl)-4-(2-(2-cyanoethoxy)-6-methoxyphenyl)-3-(2-methoxyethoxy)-2-oxo-2H-pyran-6-carboxamide (Compound 67-Ac), N-(5-(5-acetamido-1H-pyrazol-1-yl)-1,3,4-thiadiazol-2-yl)-4-(2-(2-hydroxyethoxy)-6-methoxyphenyl)-3-(2-methoxyethoxy)-2-oxo-2H-pyran-6-carboxamide (Compound 35-Ac), and N-(5-(5-amino-1H-pyrazol-1-yl)-1,3,4-thiadiazol-2-yl)-4-(2-(2-hydroxyethoxy)-6-methoxyphenyl)-3-(2-methoxyethoxy)-2-oxo-2H-pyran-6-carboxamide (Compound 35)

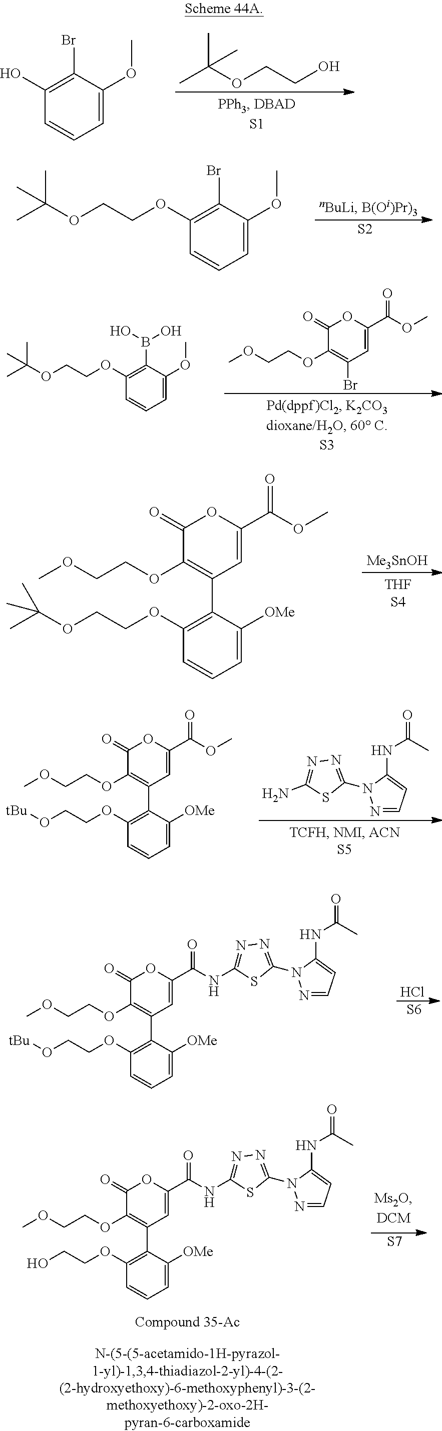

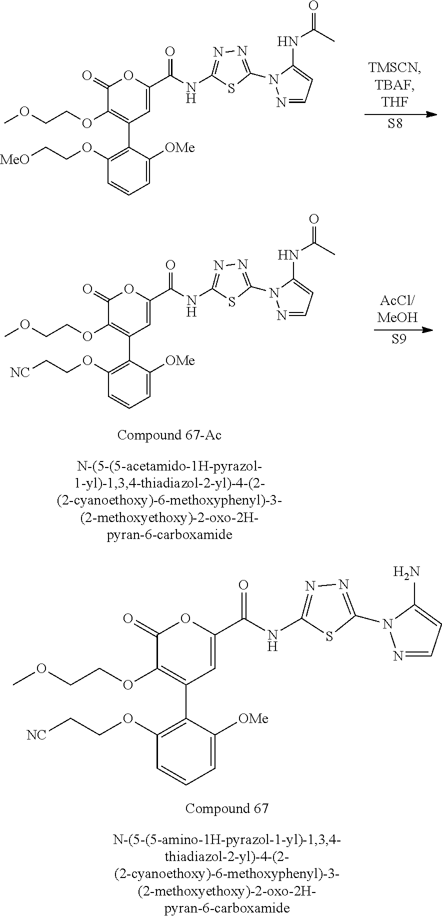

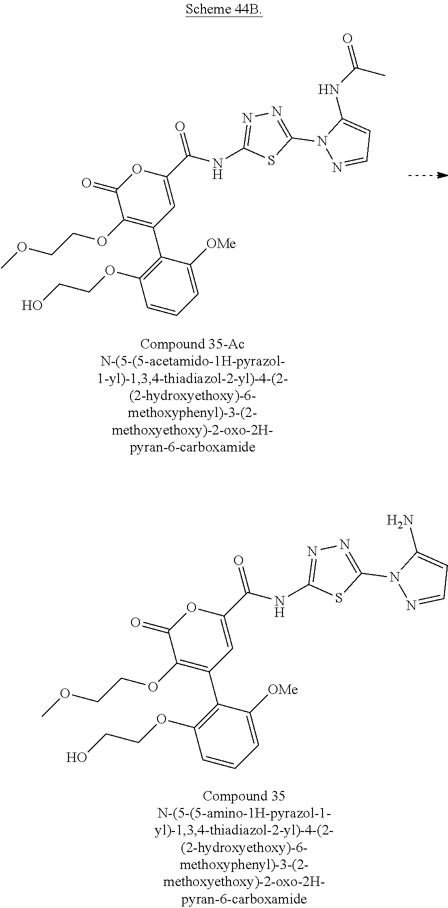

Steps 1-2: 2-bromo-1-(2-(tert-butoxy)ethoxy)-3-methoxybenzene was prepared according Step 1 of Example 22 using 2-(tert-butoxy)ethan-1-ol in place of 2-methoxyethanol. To a stirred solution of 2-bromo-1-(2-(tert-butoxy)ethoxy)-3-methoxybenzene (1 g, 3.3 mmol, 1 equiv) in tetrahydrofuran (THF) (20 mL) was added n-butyl lithium (n-BuLi) (1.5 mL, 4.7 mmol, 1.1 equiv) dropwise at −78° C. under nitrogen atmosphere. The mixture was stirred for 1 h at −78° C. under nitrogen then triisopropyl borate (B(OiPr)₃) (1.5 mL, 0.66 mmol, 2 equiv) was added dropwise at −78° C. The resulting mixture was stirred for an additional 1 h at room temperature then quenched with methanol (MeOH) at room temperature and concentrated under reduced pressure. The crude residue was purified by C18 reverse phase flash chromatography (mobile phase, MeCN in water (10 mmol/LN NH₄HCO₃), 0% to 53% gradient in 10 min; detector, UV 254 nm) to provide 2-[2-(tert-butoxy)ethoxy]-6-methoxyphenylboronic acid (500 mg, 56% yield). LCMS (ES, m/z)=269.0 [M+H]+.

Step 3: A solution of 2-[2-(tert-butoxy)ethoxy]-6-methoxyphenylboronic acid (300 mg, 1.12 mmol, 1 equiv), K—CO₃ (464 mg, 3.36 mmol, 3equiv) and methyl 4-bromo-5-(2-methoxyethoxy)-6-oxopyran-2-carboxylate (product of Step 1, Example 3) (344 mg, 1.12 mmol, 1 equiv) in 1,4-dioxane (10 mL) and H₂O (1 mL) was stirred for 1 h at 60° C. under nitrogen atmosphere then cooled to r and concentrated under reduced pressure. The crude residue was purified by C18 revers phase flash chromatography (mobile phase, MeCN in water (10 mmol/L NH₄HCO₃), 0% to 64% gradient in 10 min; detector, UV 254 nm) to provide methyl 4-{2-[2-(tert-butoxy)ethoxy]-6-methoxyphenyl}-5-(2-methoxyethoxy)-6-oxopyran-2-carboxylate (340 mg, 67% yield). LCMS (ES, m/z)=451.0 [M+H]+.

Step 4: A solution of methyl 4-{2-[2-(tert-butoxy)ethoxy]-6-methoxyphenyl}-5-(2-methoxyethoxy)-6-oxopyran-2-carboxylate (330 mg, 0.733 mmol, 1 equiv) and trimethyltin hydroxide (265 mg, 1.47 mmol, 2 equiv) in tetrahydrofuran (THF) (5 mL) was stirred overnight at room temperature under nitrogen atmosphere. The mixture was then concentrated under reduced pressure and purified by C18 reverse phase flash chromatography (mobile phase, MeCN in water (0.1% FA), 0% to 54% gradient in 10 min; detector, UV 254 nm) to provide 4-{2-[2-(tert-butoxy)ethoxy]-6-methoxyphenyl}-5-(2-methoxyethoxy)-6-oxopyran-2-carboxylic acid (275 mg, 86% yield). LCMS (ES, m/z)=437.0 [M+H]+.

Step 5: To a stirred mixture of 4-{2-[2-(tert-butoxy)ethoxy]-6-methoxyphenyl}-5-(2-methoxyethoxy)-6-oxopyran-2-carboxylic acid (“aryl-pyrone reagent”) (260 mg, 0.596 mmol, 1 equiv) and N-(1-(5-amino-1,3,4-thiadiazol-2-yl)-1H-pyrazol-5-yl)acetamide (product of Step 3, Example 5; “ADT amine reagent”) (160 mg, 0.715 mmol, 1.2 equiv) in N,N-dimethylformamide (DMF) (5 mL) was added chloro-N,N,N′,N′-tetramethylformamidinium hexafluorophosphate (TCFH) (501 mg, 1.79 mmol, 3 equiv) in portions at room temperature under nitrogen atmosphere, and the reaction was stirred at rt until complete by LCMS. The mixture was then concentrated under reduced pressure and purified by C18 reverse phase flash chromatography (mobile phase, MeCN in water (10 mmol/L NH₄HCO₃), 0% to 33% gradient in 10 min; detector, UV 254 nm) to provide 4-{2-[2-(tert-butoxy)ethoxy]-6-methoxyphenyl}-N-[5-(5-acetamidopyrazol-1-yl)-1,3,4-thiadiazol-2-yl]-5-(2-methoxyethoxy)-6-oxopyran-2-carboxamide (320 mg, 84% yield). LCMS (ES, m/z)=643.0 [M+H]+.

Step 6: A solution of 4-{2-[2-(tert-butoxy)ethoxy]-6-methoxyphenyl}-N-[5-(5-acetamidopyrazol-1-yl)-1,3,4-thiadiazol-2-yl]-5-(2-methoxyethoxy)-6-oxopyran-2-carboxamide (300 mg, 0.467 mmol, 1 equiv) in HCl (4 mL of 4 M in 1,4-dioxane) was stirred for 2 h at room temperature then concentrated under reduced pressure to provide N-(5-(5-acetamido-1H-pyrazol-1-yl)-1,3,4-thiadiazol-2-yl)-4-(2-(2-hydroxyethoxy)-6-methoxyphenyl)-3-(2-methoxyethoxy)-2-oxo-2H-pyran-6-carboxamide (Compound 35-Ac) (220 mg, 80% yield) which was used in the next step directly without further purification. LCMS (ES, m/z)=587.2 [M+H]+.

Step 7: To a stirred solution of N-(5-(5-acetamido-1H-pyrazol-1-yl)-1,3,4-thiadiazol-2-yl)-4-(2-(2-hydroxyethoxy)-6-methoxyphenyl)-3-(2-methoxyethoxy)-2-oxo-2H-pyran-6-carboxamide (Compound 35-Ac) (70 mg, 0.12 mmol, 1 equiv) in dichloromethane (DCM) (3 mL) was added triethylamine (TEA) (36 mg, 0.36 mmol, 3 equiv), methanesulfonic anhydride (31 mg, 0.18 mmol, 1.5 equiv) dropwise at 0° C. under nitrogen atmosphere. The resulting mixture was stirred for 2 h at room temperature under nitrogen atmosphere then diluted with water (5 mL) and extracted with dichloromethane (DCM) (3×10 mL). The combined organic layers were washed with brine, dried over anhydrous Na₂SO₄, filtered, and concentrated under reduced pressure to provide 2-[2-(6-{[5-(5-acetamidopyrazol-1-yl)-1,3,4-thiadiazol-2-yl]carbamoyl}-3-(2-methoxyethoxy)-2-oxopyran-4-yl)-3-methoxyphenoxy]ethyl methanesulfonate (90 mg, 100% yield), which was used directly in the next step without further purification. LCMS (ES, m/z)=665.1 [M+H]+.

Step 8: To a stirred solution of 2-[2-(6-{[5-(5-acetamidopyrazol-1-yl)-1,3,4-thiadiazol-2-yl]carbamoyl}-3-(2-methoxyethoxy)-2-oxopyran-4-yl)-3-methoxyphenoxy]ethyl methanesulfonate (80 mg, 0.12 mmol, 1 equiv) in tetrahydrofuran (THF) (2 mL) was added tetrabutylammonium fluoride (TBAF) (47 mg, 0.18 mmol, 1.5 equiv) and trimethylsilyl cyanide (TMSCN) (24 mg, 0.24 mmol, 2 equiv) at room temperature under nitrogen atmosphere. The resulting mixture was stirred for 1 h at room temperature then diluted with water (5 mL) and extracted with ethyl acetate (EtOAc) (3×10 mL). The combined organic layers were washed with brine, dried over anhydrous Na₂SO₄, filtered, and concentrated under reduced pressure. The resulting residue was purified by C18 reverse phase flash chromatography (mobile phase, MeCN in water (0.1% TFA), 10% to 50% gradient in 10 min: detector. UV 254 nm) to provide N-(5-(5-acetamido-1H-pyrazol-1-yl)-1,3,4-thiadiazol-2-yl)-4-(2-(2-cyanoethoxy)-6-methoxyphenyl)-3-(2-methoxyethoxy)-2-oxo-2H-pyran-6-carboxamide (Compound 67-Ac) (36 mg, 50% yield). LCMS (ES, m/z)=596.2 [M+H]+.

Step 9: To a stirred solution of N-(5-(5-acetamido-1H-pyrazol-1-yl)-1,3,4-thiadiazol-2-yl)-4-(2-(2-cyanoethoxy)-6-methoxyphenyl)-3-(2-methoxyethoxy)-2-oxo-2H-pyran-6-carboxamide (Compound 67-Ac) (30 mg, 0.05 mmol, 1 equiv) in MeOH (3 mL) was added AcCl (8 mg, 0.10 mmol, 2 equiv) at room temperature under nitrogen atmosphere. The resulting mixture was stirred for 2 h at room temperature then concentrated under reduced pressure. The crude residue was purified by C18 reverse phase flash chromatography (conditions: column, C18; mobile phase, MeCN in Water (10 mmol/L TFA), 10% to 50% gradient in 10 min; detector, UV 254 nm) to provide N-(5-(5-amino-1H-pyrazol-1-yl)-1,3,4-thiadiazol-2-yl)-4-(2-(2-cyanoethoxy)-6-methoxyphenyl)-3-(2-methoxyethoxy)-2-oxo-2H-pyran-6-carboxamide (Compound 67) (4.1 mg, 14% yield). LCMS (ES, m/z)=554.3 [M+H]+; ¹H NMR (300 MHz, DMSO-d6) δ 7.43-7.38 (m, 2H), 7.06 (s, 1H), 6.83-6.80 (m, 2H), 6.62 (br, 2H), 5.40 (d, J=1.8 Hz, 1H), 4.21-4.13 (m, 4H), 3.77 (s, 3H), 3.38 (t, J=4.8 Hz, 2H), 3.10 (s, 3H), 2.88 (t, J=4.8 Hz, 2H).

N-(5-(5-amino-1H-pyrazol-1-yl)-1,3,4-thiadiazol-2-yl)-4-(2-(2-hydroxyethoxy)-6-methoxyphenyl)-3-(2-methoxyethoxy)-2-oxo-2H-pyran-6-carboxamide (Compound 35) may be prepared following above Step 9 of Example 44 by acidic deprotection of N-(5-(5-acetamido-1H-pyrazol-1-yl)-1,3,4-thiadiazol-2-yl)-4-(2-(2-hydroxyethoxy)-6-methoxyphenyl)-3-(2-methoxyethoxy)-2-oxo-2H-pyran-6-carboxamide (Compound 35-Ac).

### Example 45: N-(5-(5-amino-1H-pyrazol-1-yl)-1,3,4-thiadiazol-2-yl)-4-(2,6-dimethoxyphenyl)-3-(2-(2-hydroxyethoxy)ethoxy)-2-oxo-2H-pyran-6-carboxamide (Compound 68)

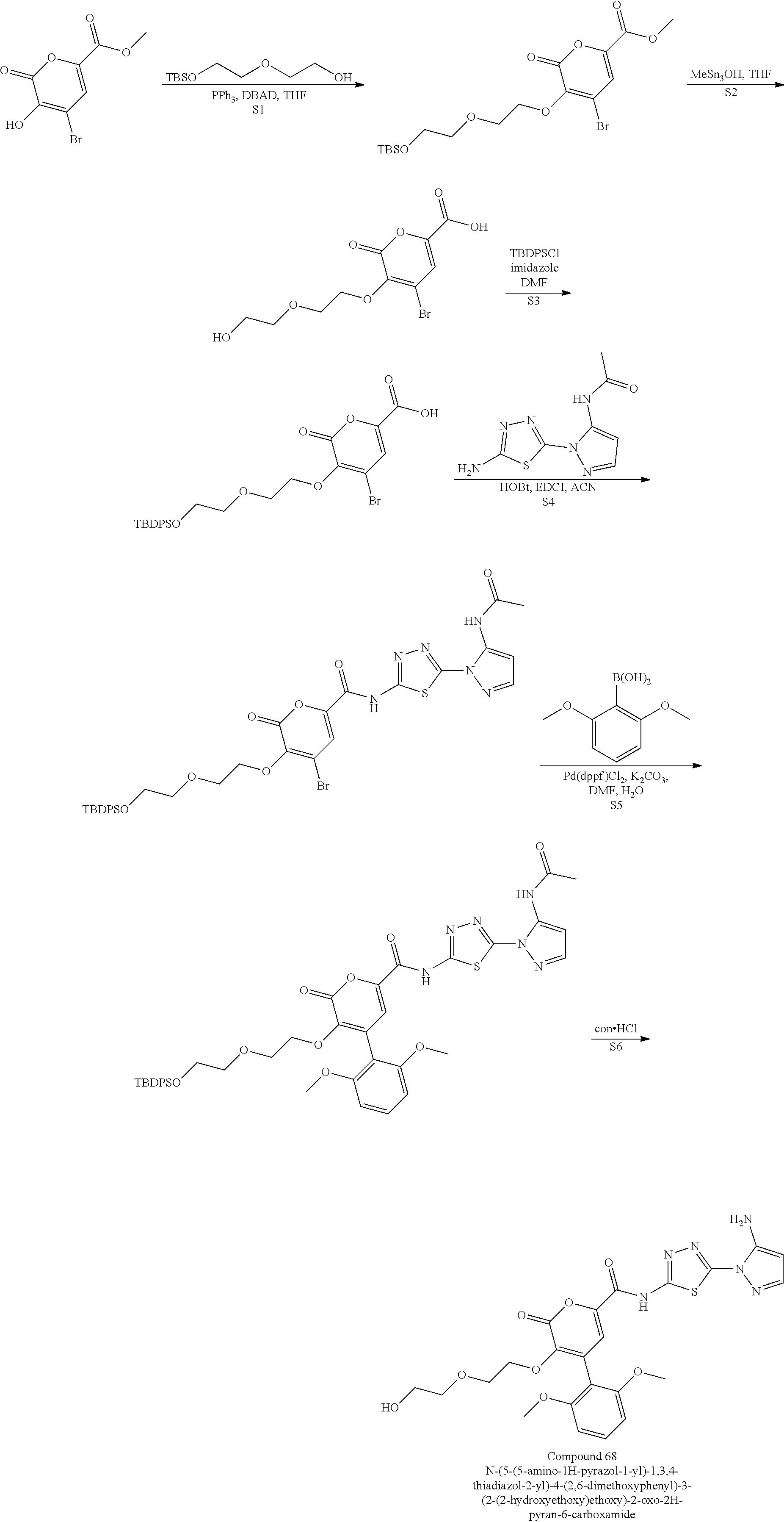

Step 1: Methyl 4-bromo-5-(2-{2-[(tert-butyldimethylsilyl)oxy]ethoxy}ethoxy)-6-oxopyran-2-carboxylate was prepared according to Step 2 of Example 27 using 2-(2-[(tert-butyldimethylsilyl)oxy]ethoxy}ethanol in place of 2,2-dimethylpropane-1,3-diol and di-tert-butyl azodicarboxylate (DBAD) in place of diethyl azodicarboxylate (DEAD). LCMS (ES, m/z)=451.1 [M+H]+.

Step 2: 4-Bromo-3-(2-(2-hydroxyethoxy)ethoxy)-2-oxo-2H-pyran-6-carboxylic acid was prepared according to Step 3 of Example 27 using methyl 4-bromo-5-(2-{2-[(tert-butyldimethylsilyl)oxy]ethoxy}ethoxy)-6-oxopyran-2-carboxylate in place of methyl 4-bromo-5-{3-[(tert-butyldiphenylsilyl)oxy]-2,2-dimethylpropoxy}-6-oxopyran-2-carboxylate. LCMS (ES, m/z)=559.1 [M+H]+.

Step 3: To a stirred solution of 4-bromo-5-[2-(2-hydroxyethoxy)ethoxy]-6-oxopyran-2-carboxylic acid (450 mg, 1.39 mmol, 1 equiv) in N,N-dimethylformamide (DMF) (5 mL) was added tert-butyl(chloro)diphenylsilane (TBDPSCl) (766 mg, 2.79 mmol, 2 equiv) and imidazole (284 mg, 4.18 mmol, 3 equiv) at room temperature. The resulting mixture was stirred for 10 h at room temperature then diluted with water (30 mL) and extracted with ethyl acetate (EtOAc) (3×50 mL). The combined organic layers were washed with brine (2×10 mL), dried over anhydrous Na₂SO₄, filtered, and concentrated under reduced pressure. The crude residue was purified by C18 reverse phase flash chromatography (MeCN in water, 10% to 50% gradient in 10 min; detector. UV 254 nm) to provide 4-bromo-5-(2-{2-[(tert-butyldiphenylsilyl)oxy]ethoxy}ethoxy)-6-oxopyran-2-carboxylic acid (300 mg, 38% yield). LCMS (ES, m/z)=561.1 [M+H]+.

Steps 4-6: N-(5-(5-amino-1H-pyrazol-1-yl)-1,3,4-thiadiazol-2-yl)-4-(2,6-dimethoxyphenyl)-3-(2-(2-hydroxyethoxy)ethoxy)-2-oxo-2H-pyran-6-carboxamide (Compound 68) was prepared according to Steps 3-5 of Example 36 using 4-bromo-5-(2-{2-[(tert-butyldiphenylsilyl)oxy]ethoxy}ethoxy)-6-oxopyran-2-carboxylic acid in place of 4-bromo-5-([(2S)-1-methoxypropan-2-yl]oxy-6-oxopyran-2-carboxylic acid. LCMS (ES, m/z)=545.1 [M+H]+: ¹H NMR (400 MHz, DMSO-d6) δ 13.20 (br, 1H), 7.48 (d, J=2.0 Hz, 1H), 7.43 (d, J=8.4 Hz, 1H), 7.29 (s, 1H), 6.79 (d, J=8.4 Hz, 2H), 6.66 (s, 2H), 5.45 (d, J=2.0 Hz, 1H), 4.49 (br, 1H), 4.22-4.16 (m, 2H), 3.78 (s, 6H), 3.48 (t, J=4.8 Hz, 2H), 3.38 (t, J=5.2 Hz, 2H), 3.27 (t, J=5.2 Hz, 2H).

### Example 46: N-(5-(5-amino-1H-pyrazol-1-yl)-1,3,4-thiadiazol-2-yl)-4-(2,6-dimethoxyphenyl)-3-(((1S,2S)-2-hydroxycyclopentyl)oxy)-2-oxo-2H-pyran-6-carboxamide (Compound 71A*), N-(5-(5-amino-1H-pyrazol-1-yl)-1,3,4-thiadiazol-2-yl)-4-(2,6-dimethoxyphenyl)-3-(((1R,2R)-2-hydroxycyclopentyl)oxy)-2-oxo-2H-pyran-6-carboxamide (Compound 71B*), and N-(5-(5-acetamido-1H-pyrazol-1-yl)-1,3,4-thiadiazol-2-yl)-4 (2,6-dimethoxyphenyl)-3-(((1,2-trans)-2-hydroxycyclopentyl)oxy)-2-oxo-2H-pyran-6-carboxamide (Compound 71-Ac)

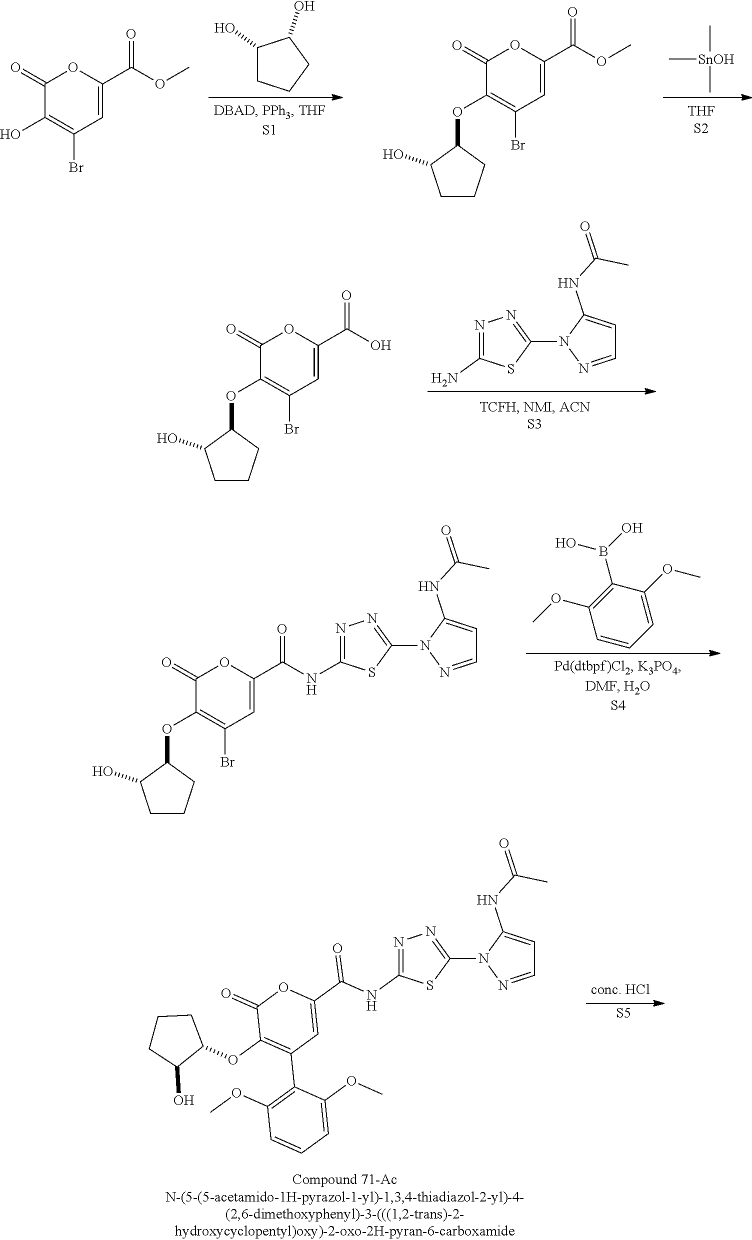

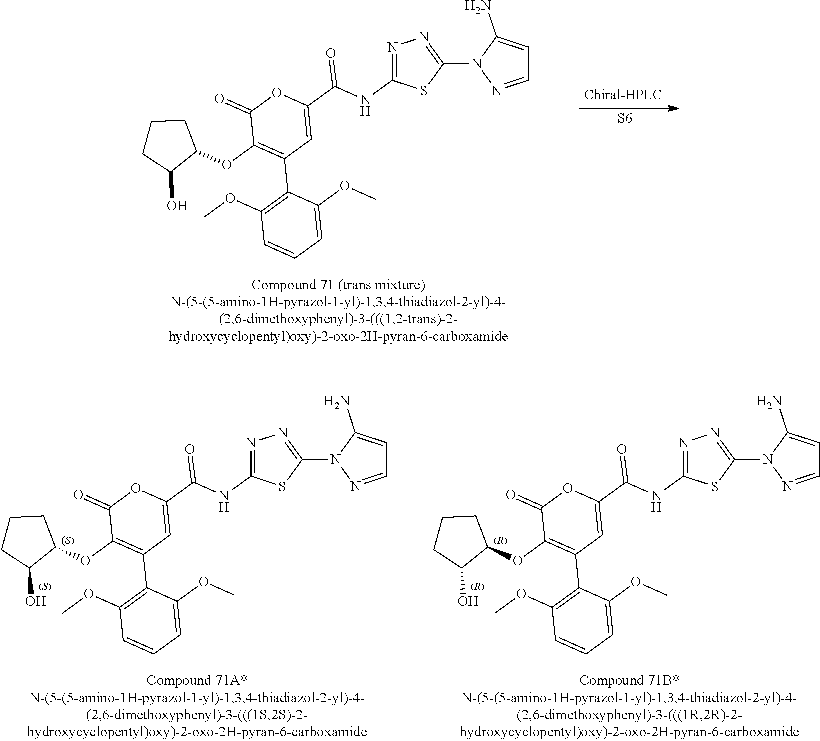

Steps 1-5: A trans mixture of N-(5-(5-amino-1H-pyrazol-1-yl)-1,3,4-thiadiazol-2-yl)-4-(2,6-dimethoxyphenyl)-3-(((1,2-trans)-2-hydroxycyclopentyl)oxy)-2-oxo-2H-pyran-6-carboxamide (Compound 71) was prepared according to Steps 2-6 of Example 13 using (1,2-cis)-cyclopentane-1,2-diol in place of 2-(t-butoxy)ethanol and methyl 4-bromo-3-hydroxy-2-oxo-2H-pyran-6-carboxylate in place of methyl 4-iodo-3-hydroxy-2-oxo-2H-pyran-6-carboxylate.

Step 6: The trans isomers of N-(5-(5-amino-1H-pyrazol-1-yl)-1,3,4-thiadiazol-2-yl)-4-(2,6-dimethoxyphenyl)-3-(((1,2-trans)-2-hydroxycyclopentyl)oxy)-2-oxo-2H-pyran-6-carboxamide (Compound 71) was separated by chiral prep-HPLC (CHIRALPAK IE, 2*25 cm; Mobile Phase A: Hexanes (0.1% TFA), Mobile Phase B: MeOH:DCM=1:1; Flow rate: 20 mL/min; Gradient: 60% B to 60% B in 14 min; Wave Length: 220/254 nm) to afford N-(5-(5-amino-1H-pyrazol-1-yl)-1,3,4-thiadiazol-2-yl)-4-(2,6-dimethoxyphenyl)-3-(((1S,2S)-2-hydroxycyclopentyl)oxy)-2-oxo-2H-pyran-6-carboxamide (Compound 71A*) as the first eluting peak (chiral RT=10.6 min) and N-(5-(5-amino-1H-pyrazol-1-yl)-1,3,4-thiadiazol-2-yl)-4-(2,6-dimethoxyphenyl)-3-(((1R,2R)-2-hydroxycyclopentyl)oxy)-2-oxo-2H-pyran-6-carboxamide (Compound 71B*) as the second eluting peak (chiral RT=13.18 min). Stereochemistry arbitrarily assigned.

Compound 71A*: LCMS (ES, m/z)=541.1 [M+H]+, ¹H NMR (40 MHz, DMSO-d6) δ 7.59 (s, 1H), 7.48 (d, J=6.0 Hz, 1H), 7.29 (s, 1H), 6.79-6.77 (m, 2H), 5.46 (d, J=2.0 Hz, 1H), 4.72 (d, J=4.8 Hz, 1H), 3.82-3.75 (m, 1H), 3.71 (s, 6H), 1.67-1.51 (m, 1H), 1.43-1.43 (m, 2H), 1.20-1.09 (m, 3H).

Compound 71B*: LCMS (ES, m/z)=541.1 [M+H]+, ¹H NMR (400 MHz, DMSO-d6) δ 7.59 (s, 1H), 7.48 (d, J=6.0 Hz, 1H), 7.29 (s, 1H), 6.79-6.77 (m, 2H), 5.46 (d, J=2.0 Hz, 1H), 4.72 (d, J=4.8 Hz, 1H), 3.82-3.75 (m, 1H), 3.71 (s, 6H), 1.67-1.51 (m, 1H), 1.43-1.43 (m, 2H), 1.20-1.09 (m, 3H).

### Example 47: N-(5-(5-amino-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 73) and N-(5-(5-acetamido-1H-pyrazol-1-yl)-1,3,4-thiadiazol-2-yl)-3-(2-methoxy-2-methylpropoxy)-4-(3-methoxypyridin-2-yl)-2-oxo-2H-pyran-6-carboxamide (Compound 73-Ac)

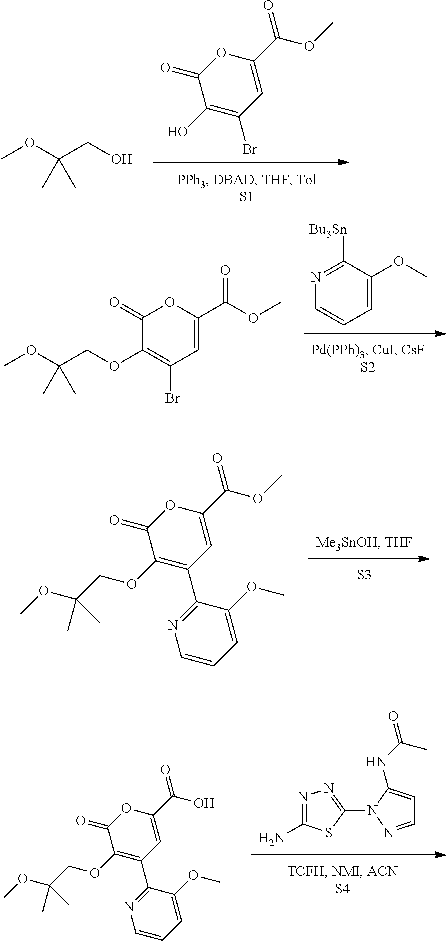

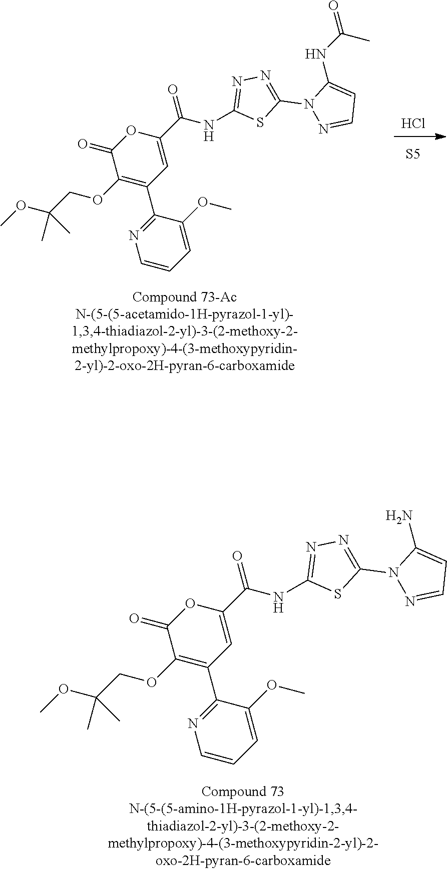

Step 1: Methyl 4-bromo-5-(2-methoxy-2-methylpropoxy)-6-oxopyran-2-carboxylate was prepared according to Step 1 of Example 24 using 2-methoxy-2-methylpropan-1-ol in place of 3-methoxy-2,2-dimethylpropan-1-ol and methyl 5-hydroxy-4-bromo-6-oxopyran-2-carboxylate in place of methyl 5-hydroxy-4-iodo-6-oxopyran-2-carboxylate. ¹H NMR (400 MHz, Chloroform-d) δ 7.31 (s, 1H), 4.28 (s, 2H), 3.94 (s, 3H), 3.28 (s, 3H), 1.31 (s, 6H).

Step 2: Methyl 5-(2-methoxy-2-methylpropoxy)-4-(3-methoxypyridin-2-yl)-6-oxopyran-2-carboxylate was prepared according to Step 4 of Example 24 using methyl 4-bromo-5-(2-methoxy-2-methylpropoxy)-6-oxopyran-2-carboxylate in place of N-[5-(5-acetamidopyrazol-1-yl)-1,3,4-thiadiazol-2-yl]-4-iodo-5-(3-methoxy-2,2-dimethylpropoxy)-6-oxopyran-2-carboxamide. LCMS (ES, m/z)=364.0 [M+H]+.

Step 3: 5-(2-methoxy-2-methylpropoxy)-4-(3-methoxypyridin-2-yl)-6-oxopyran-2-carboxylic acid was prepared according to Step 2 of Example 24 using methyl 5-(2-methoxy-2-methylpropoxy)-4-(3-methoxypyridin-2-yl)-6-oxopyran-2-carboxylate in place of methyl 4-iodo-5-(3-methoxy-2,2-dimethylpropoxy)-6-oxopyran-2-carboxylate. LCMS (ES, m/z)=350.1 [M+H]+.

Steps 4-5: N-(5-(5-amino-1H-pyrazol-1-yl)-1,3,4-thiadiazol-2-yl)-3-(3-methoxy-2,2-dimethylpropoxy)-4-(3-methoxypyridin-2-yl)-2-oxo-2H-pyran-6-carboxamide (Compound 73) was prepared according to Steps 4-5 of Example 16 using 5-(2-methoxy-2-methylpropoxy)-4-(3-methoxypyridin-2-yl)-6-oxopyran-2-carboxylic acid (“aryl-pyrone reagent”) in place of 4-(2,6-dimethoxyphenyl)-5-[(1,3-dimethoxypropan-2-yl)oxy]-6-oxopyran-2-carboxylic acid, which was coupled with N-(1-(5-amino-1,3,4-thiadiazol-2-yl)-1H-pyrazol-5-yl)acetamide (product of Step 3, Example 5; “ADT amine reagent”) to provide N-(5-(5-acetamido-1H-pyrazol-1-yl)-1,3,4-thiadiazol-2-yl)-3-(2-methoxy-2-methylpropoxy)-4-(3-methoxypyridin-2-yl)-2-oxo-2H-pyran-6-carboxamide (Compound 73-Ac), which was then deprotected under acidic conditions. LCMS (ES, m/z)=514.2 [M+H]+: ¹H NMR (400 MHz, DMSO-d6) δ 13.30 (br, 1H), 8.30-8.29 (m, 1H), 7.66-7.64 (m, 1H), 7.56-7.45 (m, 3H), 6.57 (s, 1H), 5.45 (d, J=2.0 Hz, 1H), 3.97 (s, 2H), 3.87 (s, 3H), 2.95 (s, 3H), 0.91 (s, 6H).

### Example 48: N-(5-(5-amino-1H-pyrazol-1-yl)-1,3,4-thiadiazol-2-yl)-4-(2-cyano-6-(difluoromethoxy)phenyl)-3-(2-methoxyethoxy)-2-oxo-2H-pyran-6-carboxamide (Compound 74) and N-(5-(5-acetamido-1H-pyrazol-1-yl)-1,3,4-thiadiazol-2-yl)-4-(2-cyano-6-(difluoromethoxy)phenyl)-3-(2-methoxyethoxy)-2-oxo-2H-pyran-6-carboxamide (Compound 74-Ac)

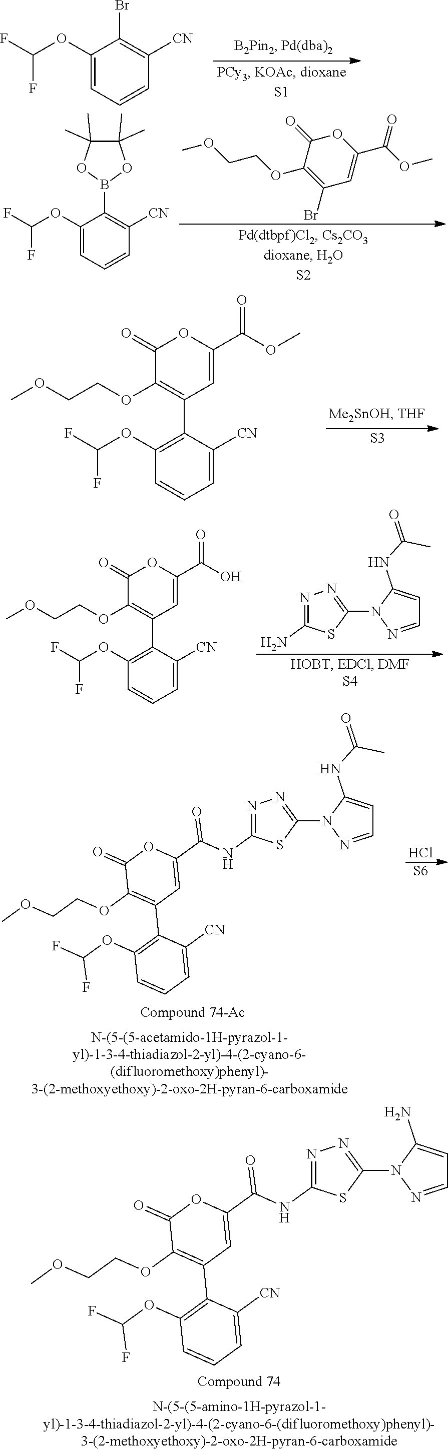

Steps 1-2: Methyl 4-[2-cyano-6-(difluoromethoxy)phenyl]-5-(2-methoxyethoxy)-6-oxopyran-2-carboxylate was prepared according to Steps 1-2 of Example 23 using 2-bromo-3-(difluoromethoxy)benzonitrile in place of 4-bromo-3,5-dimethoxypyridine. LCMS (ES, m/z)=396.0 [M+H]+.

Steps 3-5: N-(5-(5-amino-1H-pyrazol-1-yl)-1,3,4-thiadiazol-2-yl)-4-(2-cyano-6-(difluoromethoxy)phenyl)-3-(2-methoxyethoxy)-2-oxo-2H-pyran-6-carboxamide (Compound 74) was prepared according to Steps 3-5 of Example 32 using methyl 4-[2-cyano-6-(difluoromethoxy)phenyl]-5-(2-methoxyethoxy)-6-oxopyran-2-carboxylate in place of methyl 5-{3-[(tert-butyldimethylsilyl)oxy]propoxy}-4-(2,6-dimethoxyphenyl)-6-oxopyran-2-carboxylate. LCMS (ES, m/z)=546.1 [M+H]+: ¹H NMR (400 MHz, DMSO-d6) δ 7.93 (dd. J=7.2, 0.8 Hz, 1H), 7.80-7.71 (m, 2H), 7.55 (s, 1H), 7.49 (s, 1H), 7.46-7.09 (m, 1H), 4.39-4.32 (m, 2H), 3.37-3.34 (m, 2H), 3.03 (s, 3H).

### Example 49: (S)—N-(5-(5-amino-1H-pyrazol-1-yl)-1,3,4-thiadiazol-2-yl)-3-((1-hydroxypropan-2-yl)oxy)-4-(3-methoxypyridin-2-yl)-2-oxo-2H-pyran-6-carboxamide (Compound 107A) and (S)—N-(5-(5-acetamido-1H-pyrazol-1-yl)-1,3,4-thiadiazol-2-yl)-3-((1-hydroxypropan-2-yl)oxy)-4-(3-methoxypyridin-2-yl)-2-oxo-2H-pyran-6-carboxamide (Compound 107A-Ac)

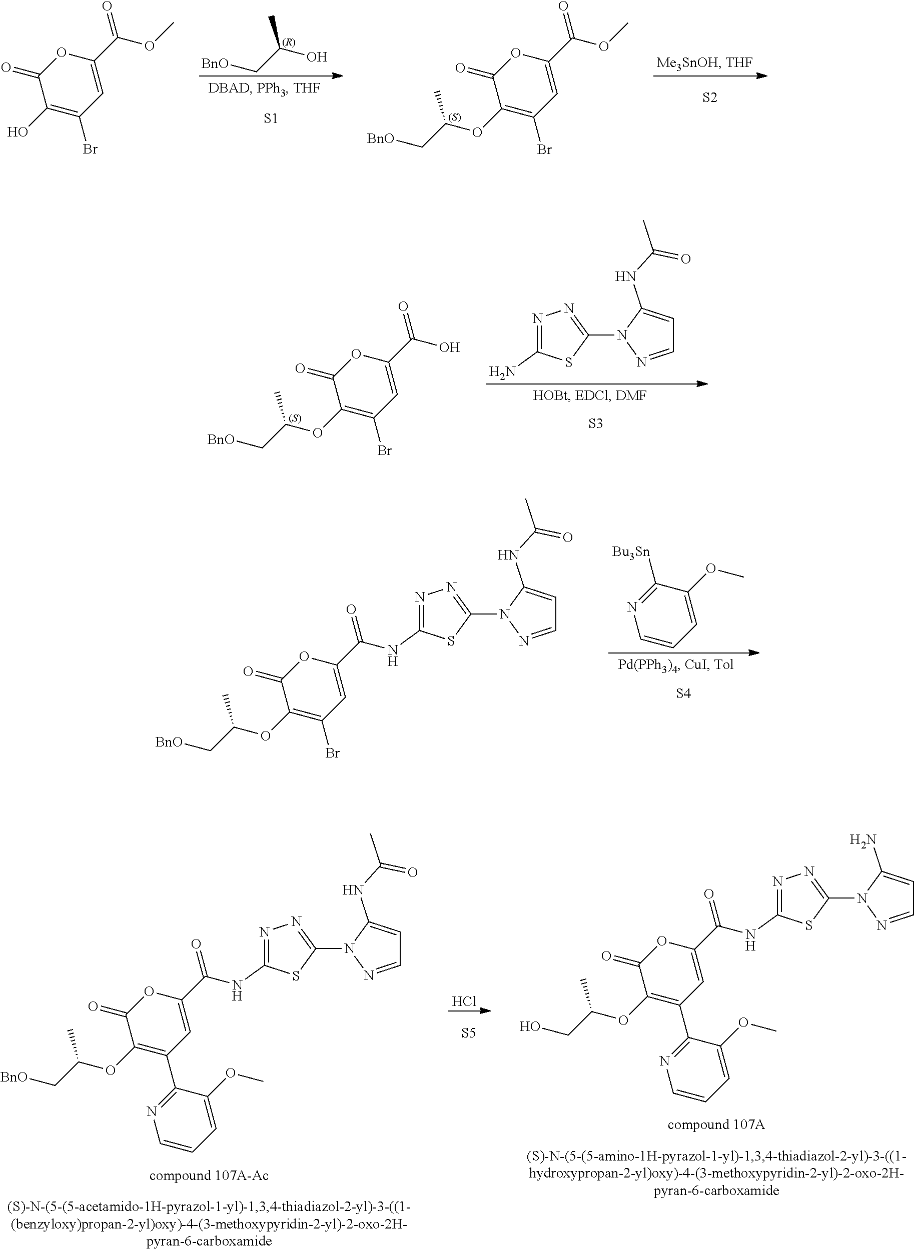

Step 1: To a stirred solution of methyl 4-bromo-5-hydroxy-6-oxopyran-2-carboxylate (4.00 g, 16.1 mmol, 1.0 equiv), (2R)-1-(benzyloxy)propan-2-ol (4.00 g, 24.1 mmol, 1.5 equiv), triphenylphosphine (PPh₃) (6.32 g, 24.1 mmol, 1.5 equiv) in tetrahydrofuran (THF) (40 mL, 494 mmol, 30.7 equiv) was added di-tert-butyl azodicarboxylate (DBAD) (5.55 g, 24.1 mmol, 1.5 equiv) at 0° C., and then the resulting mixture was stirred for 2 h at room temperature. The resulting solution was concentrated under vacuum, and then the residue was purified by reversed-phase flash chromatography with the following conditions: column, C18 silica gel; mobile phase, acetonitrile (MeCN) in water (0.1% TFA), 10% to 50% gradient in 10 min; detector, UV 254 nm. This resulted in methyl 5-{[(2S)-1-(benzyloxy)propan-2-yl]oxy}-4-bromo-6-oxopyran-2-carboxylate (7.00 g, quantitative). LCMS (ES, m/z): 397.0 [M+H]⁺.

Step 2: To a stirred solution of methyl 5-{[(2S)-1-(benzyloxy)propan-2-yl]oxy}-4-bromo-6-oxopyran-2-carboxylate (2.00 g, 5.04 mmol, 1.0 equiv) in tetrahydrofuran (THF) (20 mL) was added trimethyltin hydroxide (1.38 g, 7.58 mmol, 1.5 equiv) at room temperature, and then the resulting mixture was stirred for 2 h at room temperature. The resulting mixture was concentrated under reduced pressure. The residue was purified by reversed-phase flash chromatography with the following conditions: column, C18 silica gel; mobile phase, acetonitrile (MeCN) in water (0.1% TFA), 10/0 to 50% gradient in 10 min; detector, UV 254 nm. This resulted in 5-{[(2S)-1-(benzyloxy)propan-2-yl]oxy}-4-bromo-6-oxopyran-2-carboxylic acid (1.20 g, 62% yield). LCMS (ES, m/z): 383.0 [M+H]⁺.

Step 3: To a stirred solution of 5-{[(2S)-1-(benzyloxy)propan-2-yl]oxy}-4-bromo-6-oxopyran-2-carboxylic acid (600 mg, 1.57 mmol, 1.0 equiv), chloro-N,N,N′,N′-tetramethylformamidinium hexafluorophosphate (TCFH) (653 mg, 2.33 mmol, 1.5 equiv) and N-methylimidazole (NMI)(381 mg, 4.64 mmol, 3.0 equiv) in acetonitrile (MeCN) (10 mL) was added N-[2-(5-amino-1,3,4-thiadiazol-2-yl)pyrazol-3-yl]acetamide (279 mg, 1.24 mmol, 0.79 equiv) at room temperature. The resulting mixture was stirred for 2 h at room temperature. The precipitated solids were collected by filtration and washed with MeCN (3×5 mL) to afford 5-{[(2S)-1-(benzyloxy)propan-2-yl]oxy}-4-bromo-N-[5-(5-acetamidopyrazol-1-yl)-1,3,4-thiadiazol-2-yl]-6-oxopyran-2-carboxamide (500 mg, 54% yield). LCMS (ES, m/z): 589.1 [M+H]⁺.

Step 4: To a stirred solution of 5-{[(2S)-1-(benzyloxy)propan-2-yl]oxy}-4-bromo-N-[5-(5-acetamidopyrazol-1-yl)-1,3,4-thiadiazol-2-yl]-6-oxopyran-2-carboxamide (200 mg, 0.0170 mmol, 1.0 equiv) in toluene (2 ml) were added 3-methoxy-2-(tributylstannyl)pyridine (70 mg, 0.018 mmol, 1.0 equiv), tetrakis(triphenylphosphine)palladium(0) (Pd(PPh₃)₄) (80 mg, 0.0030 mmol, 0.20 equiv) and copper(I) iodide (40 mg, 0.0050 mmol, 0.31 equiv) at room temperature. The resulting mixture was stirred for 2 h at 100° C. The resulting mixture was filtered, the filter cake was washed with methanol (2×1 mL). After filtration, the filtrate was concentrated under reduced pressure. This resulted in 5-([(2S)-1-(benzyloxy)propan-2-yl]oxy-N-[5-(5-acetamidopyrazol-1-yl)-1,3,4-thiadiazol-2-yl]-4-(3-methoxypyridin-2-yl)-6-oxopyran-2-carboxamide (50 mg, 90% yield). LCMS (ES, m/z): 617.3 [M+1]⁺.

Step 5: A solution of 5-([(2S)-1-(benzyloxy)propan-2-yl]oxy-N-[5-(5-acetamidopyrazol-1-yl)-1,3,4-thiadiazol-2-yl]-4-(3-methoxypyridin-2-yl)-6-oxopyran-2-carboxamide (50 mg, 0.081 mmol, 1.0 equiv) in concentrated HCl (5 mL) was stirred for 1 h at room temperature. The resulting mixture was concentrated under reduced pressure. The residue was purified by reversed-phase flash chromatography with the following conditions: column, C18 silica gel; mobile phase, acetonitrile (MeCN) in water (0.1% TFA), 10% to 50% gradient in 10 min; detector, UV 254 nm. This resulted in (S)—N-(5-(5-amino-1H-pyrazol-1-yl)-1,3,4-thiadiazol-2-yl)-3-((1-hydroxypropan-2-yl)oxy)-4-(3-methoxypyridin-2-yl)-2-oxo-2H-pyran-6-carboxamide (Compound 107A) (5.7 mg, 14% yield). LCMS (ES, m/z): 486.1 [M+1]⁺. ¹H NMR (400 MHz, DMSO-d₆) δ 13.34 (br s, 1H), 8.27-8.22 (m, 1H), 7.70-7.64 (m, 1H), 7.59-7.50 (m, 3H), 6.64 (br s, 1H), 5.45 (d, J=2.0 Hz, 1H), 4.65-4.54 (m, 2H), 3.93 (s, 3H), 3.39-3.24 (m, 2H), 1.06-1.04 (m, 3H).

### Example 50: N-(5-(5-amino-1H-pyrazol-1-yl)-1,3,4-thiadiazol-2-yl)-4-(2,6-dimethoxyphenyl)-3-(((1r,4r)-4-hydroxycyclohexyl)oxy)-2-oxo-2H-pyran-6-carboxamide (Compound 108A)

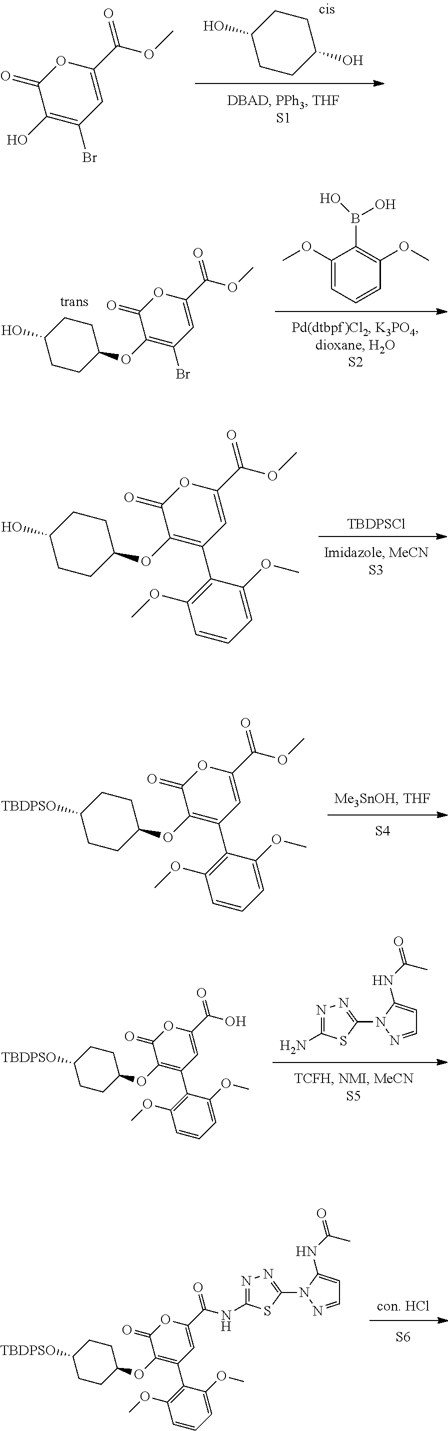

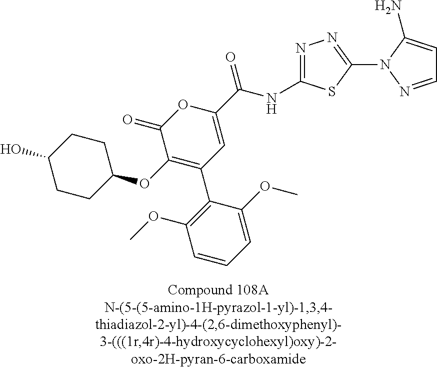 [SMILES: COc1cccc(OC)c1-c1cc(C(=O)Nc2nnc(-n3nccc3N)s2)oc(=O)c1O[C@H]1CC[C@H](O)CC1]

Step 1: To a stirred solution of methyl 4-bromo-5-hydroxy-6-oxopyran-2-carboxylate (1.00 g, 4.02 mmol, 1.0 equiv), triphenylphosphine (PPh₃) (1.58 g, 6.02 mmol, 1.5 equiv) and cis-(1s,4s)-cyclohexane-1,4-diol (0.930 g, 8.03 mmol, 2.0 equiv) in tetrahydrofuran (THF) (20 mL) were added di-tert-butyl azodicarboxylate (DBAD) (1.39 g, 6.02 mmol, 1.5 equiv) dropwise at 0° C. under nitrogen atmosphere. The resulting mixture was stirred for overnight at room temperature under nitrogen atmosphere. The resulting mixture was diluted with water (50 mL), extracted with ethyl acetate (EtOAc) (3×50 mL). The combined organic layers were washed with brine (30 mL), dried over anhydrous Na₂SO₄. After filtration, the filtrate was concentrated under reduced pressure. The residue was purified by silica gel column chromatography, eluted with petroleum ether/ethyl acetate (1:1) to afford methyl 4-bromo-6-oxo-5-([trans-4-hydroxycyclohexyl]oxypyran-2-carboxylate (560 mg, 40% yield). LCMS (ES, m/z): 347.0 [M+H]⁺.

Step 2: A solution of methyl 4-bromo-6-oxo-5-([trans-4-hydroxycyclohexyl]oxypyran-2-carboxylate (400 mg, 1.15 mmol, 1 equiv), [1,1′-Bis(di-tert-butylphosphino) ferrocene]dichloropalladium(II) (Pd(DtBPF)Cl₂) (158 mg, 0.242 mmol, 0.21 equiv), K₃PO₄ (480 mg, 2.26 mmol, 2.0 equiv) and 2,6-dimethoxyphenylboronic acid (321 mg, 1.76 mmol, 1.5 equiv) in dioxane (6 mL) and H₂O (1 mL) was stirred for 1 h at 80° C. under nitrogen atmosphere. The resulting mixture was diluted with water (50 mL), extracted with ethyl acetate (3×50 mL). The combined organic layers were washed with brine (1×20 mL), dried over anhydrous Na₂SO₄. After filtration, the filtrate was concentrated under reduced pressure. The residue was purified by silica gel column chromatography, eluted with petroleum ether/ethyl acetate (1:1) to afford methyl 4-(2,6-dimethoxyphenyl)-6-oxo-5-([trans-4-hydroxycyclohexyl]oxypyran-2-carboxylate (290 mg, 62% yield). LCMS (ES, m/z): 405.1 [M+H]⁺.

Step 3: To a solution of methyl 4-(2,6-dimethoxyphenyl)-6-oxo-5-([trans-4-hydroxycyclohexyl]oxypyran-2-carboxylate (280 mg, 0.692 mmol, 1.0 equiv) and imidazole (95 mg, 1.40 mmol, 2.0 equiv) in acetonitrile (5 mL) was added tert-butyldiphenylchlorosilane (TBDPSCl) (286 mg, 1.04 mmol, 1.5 equiv) at room temperature, and then the resulting solution was stirred for 2 h at room temperature. The resulting mixture was diluted with water (30 mL), extracted with ethyl acetate (3×30 mL). The combined organic layers were washed with brine (1×15 mL), dried over anhydrous Na₂SO₄. After filtration, the filtrate was concentrated under reduced pressure. The residue was purified by silica gel column chromatography, eluted with petroleum ether/ethyl acetate (5:1), to afford methyl 4-(2,6-dimethoxyphenyl)-6-oxo-5-([trans-4-[(tert-butyldiphenylsilyl)oxy]cyclohexyl]oxypyran-2-carboxylate (330 mg, 74% yield). LCMS (ES, m/z): 643.2 [M+H]+.

Step 4: To a stirred solution of methyl 4-(2,6-dimethoxyphenyl)-6-oxo-5-([trans-4-[(tert-butyldiphenylsilyl)oxy]cyclohexyl]oxypyran-2-carboxylate (320 mg, 0.498 mmol, 1.0 equiv) in tetrahydrofuran (THF) (7 mL) was added trimethyltin hydroxide (180 mg, 0.995 mmol, 2.0 equiv) dropwise at room temperature. The resulting mixture was stirred for overnight at room temperature. The resulting mixture was concentrated under reduced pressure. The residue was purified by reversed-phase flash chromatography with the following conditions: column, C18 silica gel; mobile phase, acetonitrile (MeCN) in water (0.05% TFA), 10% to 50% gradient in 10 min; detector, UV 254 nm. This resulted in 4-(2,6-dimethoxyphenyl)-6-oxo-5-([(1r,4r)-4-[(tert-butyldiphenylsilyl)oxy]cyclohexyl]oxypyran-2-carboxylic acid (270 mg, 86% yield). LCMS (ES, m/z): 629.2 [M+H]⁺.

Step 5: To a solution of 4-(2,6-dimethoxyphenyl)-6-oxo-5-([(1r,4r)-4-[(tert-butyldiphenylsilyl)oxy]cyclohexyl]oxypyran-2-carboxylic acid (250 mg, 0.398 mmol, 1.0 equiv) in acetonitrile (MeCN) (5 mL) was added N-[2-(5-amino-1,3,4-thiadiazol-2-yl)pyrazol-3-yl]acetamide (90 mg, 0.401 mmol, 1.01 equiv), chloro-N,N,N′,N′-tetramethylformamidinium hexafluorophosphate (TCFH) (167 mg, 0.595 mmol, 1.5 equiv) and N-methylimidazole (NMI) (163 mg, 1.98 mmol, 5.0 equiv) at room temperature, and then the resulting solution was stirred for 1 h at room temperature. The reaction was quenched by the addition of water (20 mL) at room temperature. The precipitated solids were collected by filtration and washed with water (3×2 mL). This resulted in 4-(2,6-dimethoxyphenyl)-N-[5-(5-acetamidopyrazol-1-yl)-1,3,4-thiadiazol-2-yl]-6-oxo-5-([trans-4-[(tert-butyldiphenylsilyl)oxy]cyclohexyl]oxypyran-2-carboxamide (300 mg, 90% yield). LCMS (ES, m/z): 835.2 [M+H]+.

Step 6: A solution of 4-(2,6-dimethoxyphenyl)-N-[5-(5-acetamidopyrazol-1-yl)-1,3,4-thiadiazol-2-yl]-6-oxo-5-([trans-4-[(tert-butyldiphenylsilyl)oxy]cyclohexyl]oxypyran-2-carboxamide (100 mg, 0.120 mmol, 1.0 equiv) in concentrated HCl (3 mL) was stirred for 4 h at room temperature. The resulting mixture was concentrated under reduced pressure. The residue was purified by reversed-phase flash chromatography with the following conditions: column, C18 silica gel; mobile phase, acetonitrile (MeCN) in water (0.05% TFA), 20% to 50% gradient in 10 min: detector, UV 254 nm. This resulted in N-(5-(5-amino-1H-pyrazol-1-yl)-1,3,4-thiadiazol-2-yl)-4-(2,6-dimethoxyphenyl)-3-(((1r,4r)-4-hydroxycyclohexyl)oxy)-2-oxo-2H-pyran-6-carboxamide (Compound 108A) (15.9 mg, 23% yield). LCMS (ES, m/z): 555.1 [M+H]⁺; ¹H NMR (400 MHz, DMSO-d₆) δ 13.19 (s, 1H), 7.48-7.41 (m, 2H), 7.29 (s, 1H), 6.79 (d, J=8.4 Hz, 2H), 6.65 (s, 2H), 5.45 (d, J=2.0 Hz, 1H), 4.38-4.28 (m, 2H), 3.76 (s, 6H), 3.38-3.33 (m, 1H), 1.80-1.65 (m, 2H), 1.55-1.43 (m, 2H), 1.24-0.98 (m, 4H).

### Example 51: N-(5-(5-amino-1H-pyrazol-1-yl)-1,3,4-thiadiazol-2-yl)-3-(((1R,3S)-3-hydroxycyclopentyl)oxy)-4-(3-methoxypyridin-2-yl)-2-oxo-2H-pyran-6-carboxamide (Compound 111A*) and N-(5-(5-amino-1H-pyrazol-1-yl)-1,3,4-thiadiazol-2-yl)-3-(((1S,3R)-3-hydroxycyclopentyl)oxy)-4-(3-methoxypyridin-2-yl)-2-oxo-2H-pyran-6-carboxamide (Compound 111B*)

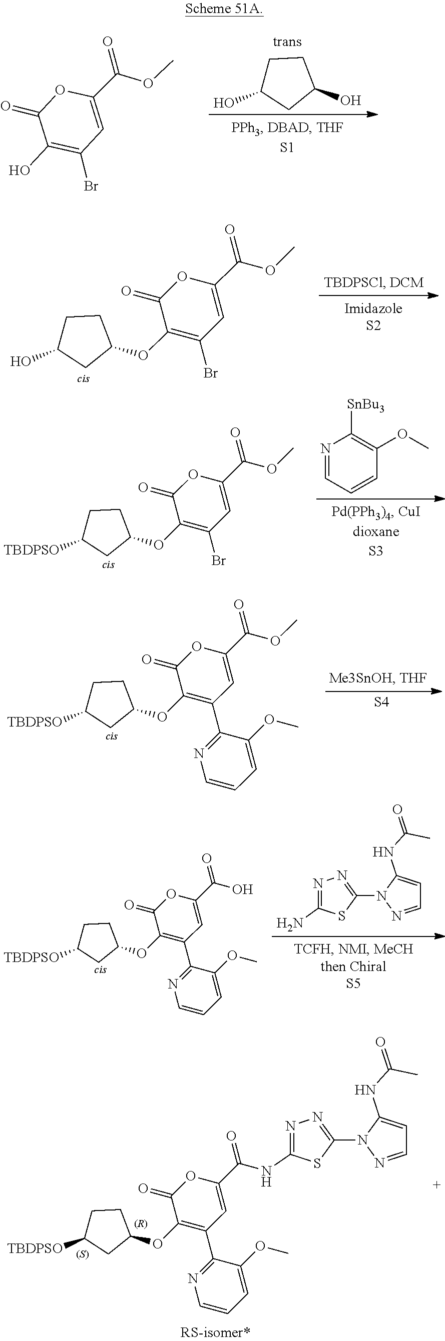

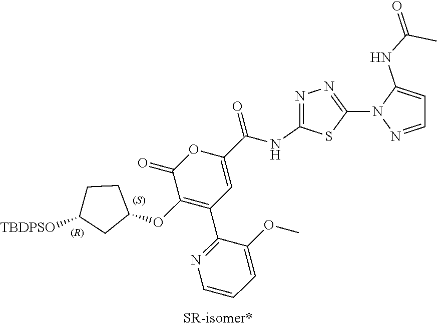

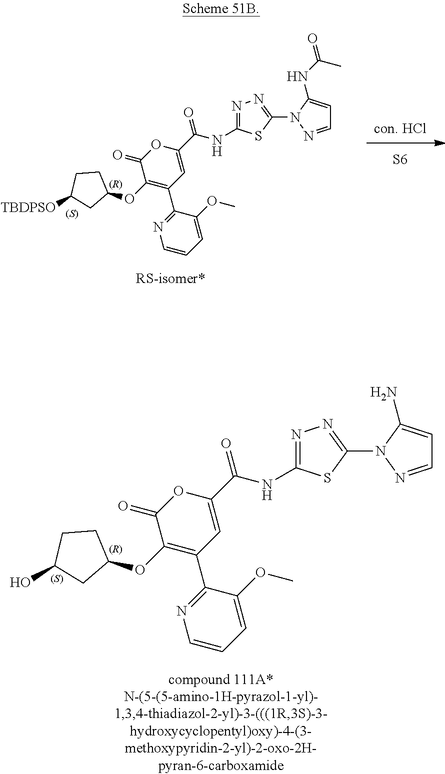

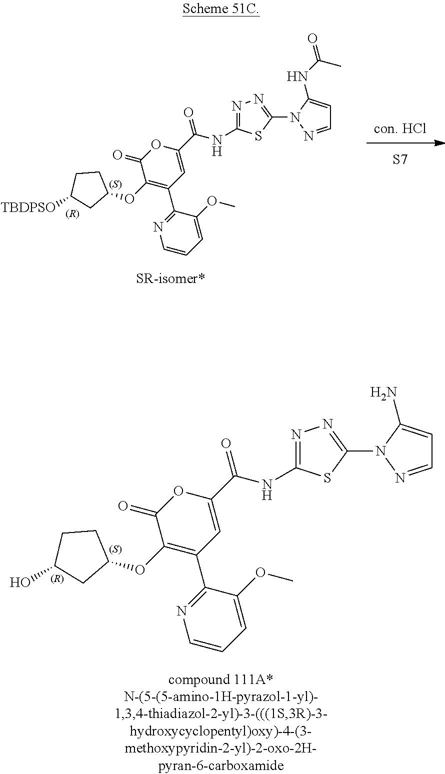

Step 1: To a solution of methyl 4-bromo-5-hydroxy-6-oxopyran-2-carboxylate (1.3 g, 5.221 mmol, 1.00 equiv), trans-cyclopentane-1,3-diol (1.00 g, 9.79 mmol, 1.9 equiv) and triphenylphosphine (PPh₃) (2.06 g, 7.85 mmol, 1.5 equiv) in tetrahydrofuran (THF) (20 mL) was added di-tert-butyl azodicarboxylate (DBAD) (1.80 g, 7.82 mmol, 1.5 equiv) at 0° C. under nitrogen, and then the resulting mixture was stirred for 48 h at 50° C. The reaction was diluted with water (100 mL) at room temperature, extracted with ethyl acetate (3×80 mL). The combined organic layers were dried over anhydrous Na₂SO₄. After filtration, the filtrate was concentrated under reduced pressure. The residue was purified by silica gel column chromatography, eluted with petroleum ether (PE)/ethyl acetate (EA) (2:3) to afford methyl 4-bromo-5-{[cis-3-hydroxycyclopentyl]oxy}-6-oxopyran-2-carboxylate (1.3 g, 71% yield). LCMS (ES, m/z): 332.9 [M+H]⁺.

Step 2: To a solution of methyl 4-bromo-5-{[cis-3-hydroxycyclopentyl]oxy}-6-oxopyran-2-carboxylate (1.3 g, 3.902 mmol, 1 equiv) and Imidazole (540 mg, 7.932 mmol, 2.03 equiv) in DCM (15 mL) was added TBDPSCl (1.7 g, 6.185 mmol, 1.58 equiv) at room temperature, and then the resulting mixture was stirred for 1 h at room temperature. The reaction was diluted with water (150 mL) at room temperature, extracted with ethyl acetate (EtOAc) (3×70 mL). The combined organic layers were dried over anhydrous Na₂SO₄. After filtration, the filtrate was concentrated under reduced pressure. The residue was purified by silica gel column chromatography, eluted with petroleum ether (PE)/ethyl acetate (EA) (1:9) to afford methyl 4-bromo-5-{[cis-3-[(tert-butyldiphenylsilyl)oxy]cyclopentyl]oxy}-6-oxopyran-2-carboxylate (1.6 g, 68% yield). LCMS (ES, m/z): 571.1[M+H]⁺.

Step 3: To a stirred solution of methyl 4-bromo-5-{[cis-3-[(tert-butyldiphenylsilyl)oxy]cyclopentyl]oxy}-6-oxopyran-2-carboxylate (500 mg, 0.875 mmol, 1 equiv), tetrakis (triphenylphosphine)palladium (0) (Pd(PPh₃)₄) (200 mg, 0.173 mmol, 0.20 equiv) and copper(I) iodide (35.0 mg, 0.184 mmol, 0.21 equiv) in dioxane (7.5 mL) was added 3-methoxy-2-(tributylstannyl)pyridine (700 mg, 1.76 mmol, 2.0 equiv) at room temperature under nitrogen, and then the resulting mixture was stirred for 1 h at 100° C. under nitrogen atmosphere. The reaction was diluted with water (50 mL) at room temperature, extracted with ethyl acetate (3×30 mL). The combined organic layers were dried over anhydrous Na₂SO₄. After filtration, the filtrate was concentrated under reduced pressure. The residue was purified by silica gel column chromatography, eluted with petroleum ether (PE)/ethyl acetate (EA) (3:7) to afford methyl 5-{[cis-3-[(tert-butyldiphenylsilyl)oxy]cyclopentyl]oxy}-4-(3-methoxypyridin-2-yl)-6-oxopyran-2-carboxylate (400 mg, 38% yield). LCMS (ES, m/z): 600.2 [M+H]⁺.

Step 4: To a stirred solution of methyl 5-{[cis-3-[(tert-butyldiphenylsilyl)oxy]cyclopentyl]oxy}-4-(3-methoxypyridin-2-yl)-6-oxopyran-2-carboxylate (400 mg, 0.667 mmol, 1.0 equiv) in tetrahydrofuran (THF) (10 mL) was added trimethylstannanol (Me₃SnOH) (200 mg, 1.11 mmol, 1.7 equiv) at room temperature, and then the resulting mixture was stirred for 1 h at 50° C. The resulting mixture was concentrated under reduced pressure. The residue was purified by reversed-phase flash chromatography with the following conditions: column, C18 silica gel; mobile phase, acetonitrile in water (0.05% TFA), 70% to 85% gradient in 15 min; detector, UV 254 nm, to afford 5-{[cis-3-[(tert-butyldiphenylsilyl)oxy]cyclopentyl]oxy}-4-(3-methoxypyridin-2-yl)-6-oxopyran-2-carboxylic acid (240 mg, 58% yield). LCMS (ES, m/z): 586.3 [M+H]⁺.

Step 5: To a stirred solution of 5-([cis-3-[(tert-butyldiphenylsilyl)oxy]cyclopentyl]oxy)-4-(3-methoxypyridin-2-yl)-6-oxopyran-2-carboxylic acid (230 mg, 0.393 mmol, 1.0 equiv), chloro-N,N,N′,N′-tetramethylformamidinium hexafluorophosphate (TCFH) (165 mg, 0.588 mmol, 1.5 equiv) and N-methyl imidazole (NMI) (97 mg, 1.181 mmol, 3.01 equiv) in acetonitrile (6 mL) was added and N-[2-(5-amino-1,3,4-thiadiazol-2-yl)pyrazol-3-yl]acetamide (69.0 mg, 0.308 mmol, 0.78 equiv) at room temperature, and then the resulting mixture was stirred for 1 h at room temperature. The precipitated solids were collected by filtration and washed with acetonitrile (5×2 mL). The resulting mixture was concentrated under reduced pressure. And the residue was purified by trituration with dimethylsulfoxide (DMSO)/acetonitrile (MeCN)/water (1:2:1), and then the precipitated solids were collected by filtration and concentrated under vacuum to afford 5-{[cis-3-[(tert-butyldiphenylsilyl)oxy]cyclopentyl]oxy}-N-[5-(5-acetamidopyrazol-1-yl)-1,3,4-thiadiazol-2-yl]-4-(3-methoxypyridin-2-yl)-6-oxopyran-2-carboxamide (120 mg, racemic) as an off-white mixture solid. And then the racemic product was separated by prep-chiral-HPLC with the following conditions (Column: CHIRAL ART Amylose-SB 2*25 cm, 5 μm; mobile phase A: hexanes (0.1% TFA), mobile phase B: 2-propanol:dichloromethane=1:1; flow rate: 20 mL/min; gradient: 50% B to 50% B in 20 min; wavelengths: 220/254 nm) to afford (N-(5-(5-acetamido-1H-pyrazol-1-yl)-1,3,4-thiadiazol-2-yl)-3-(((1R,3S)-3-((tert-butyldiphenylsilyl)oxy)cyclopentyl)oxy)-4-(3-methoxypyridin-2-yl)-2-oxo-2H-pyran-6-carboxamide (RS-isomer*; RT(min): 10.19; 60 mg; 18% yield; LCMS (ES, m/z): 792.3 [M+H]⁺) and N-(5-(5-acetamido-1H-pyrazol-1-yl)-1,3,4-thiadiazol-2-yl)-3-(((1S,3R)-3-((tert-butyldiphenylsilyl)oxy)cyclopentyl)oxy)-4-(3-methoxypyridin-2-yl)-2-oxo-2H-pyran-6-carboxamide (SR-isomer*; Chiral RT(min): 17.4; 60 mg; 18% yield; LCMS (ES, m/z): 792.3 [M+H]+). Stereochemistry arbitrarily assigned.

Step 6: A solution of the RS-isomer* (60 mg, 0.076 mmol, 1.0 equiv) in con. HCl (4 mL) was stirred for 5 h at room temperature. The resulting mixture was concentrated under reduced pressure. The residue was purified by reversed-phase flash chromatography with the following conditions: column, C18 silica gel; mobile phase, acetonitrile in water (0.05% TFA), 28% to 38% gradient in 10 min; detector, UV 254 nm, to afford N-(5-(5-amino-1H-pyrazol-1-yl)-1,3,4-thiadiazol-2-yl)-3-(((1R,3S)-3-hydroxycyclopentyl)oxy)-4-(3-methoxypyridin-2-yl)-2-oxo-2H-pyran-6-carboxamide (Compound 111A*) (21.7 mg, 56% yield).

Step 7: A solution of the SR-isomer* (60 mg, 0.076 mmol, 1.0 equiv) in con. HCl (4 mL) was stirred for 5 h at room temperature. The resulting mixture was concentrated under reduced pressure. The residue was purified by reversed-phase flash chromatography with the following conditions: column, C18 silica gel; mobile phase, acetonitrile in water (0.05% TFA), 28% to 38% gradient in 15 min: detector. UV 254 nm, to afford N-(5-(5-amino-1H-pyrazol-1-yl)-1,3,4-thiadiazol-2-yl)-3-(((1S,3R)-3-hydroxycyclopentyl)oxy)-4-(3-methoxypyridin-2-yl)-2-oxo-2H-pyran-6-carboxamide (Compound 111B*) (23.9 mg, 61% yield).

Compound 111A*: LCMS (ES, m/z): 512.1[M+H]⁺. ¹H NMR (400 MHz, DMSO-dt) S 13.26 (s, 1H), 8.29 (d, J=4.8 Hz, 1H), 7.66 (d, J=8.4 Hz, 1H), 7.53 (dd. J=8.4, 4.8 Hz, 1H), 7.48 (d, J=1.6 Hz, 2H), 6.65 (s, 2H), 5.45 (d, J=2.0 Hz, 1H), 4.92-4.89 (m, 1H), 3.94-3.90 (n, 1H), 3.87 (s, 3H), 3.41-3.35 (m, 1H), 2.12-2.06 (m, 1H), 1.59-1.55 (m, 3H), 1.42-1.38 (m, 1H), 1.23-1.15 (m, 1H).

Compound 111B*: LCMS (ES, m/z): 512.1 [M+H]⁺. ¹H NMR (400 MHz, DMSO-d₆) δ 13.26 (s, 1H), 8.29 (d, J=4.8 Hz, 1H), 7.66 (d, J=8.4 Hz, 1H), 7.53 (dd, J=8.4, 4.8 Hz, 1H), 7.48 (d, J=1.6 Hz, 2H), 6.65 (s, 2H), 5.45 (d, J=2.0 Hz, 1H), 4.92-4.89 (m, 1H), 3.94-3.90 (m, 1H), 3.87 (s, 3H), 3.41-3.35 (m, 1H), 2.12-2.06 (m, 1H), 1.59-1.55 (m, 3H), 1.42-1.38 (m, 1H), 1.23-1.15 (m, 1H).

### Example 52: N-(5-(5-amino-1H-pyrazol-1-yl)-1,3,4-thiadiazol-2-yl)-3-(((1R,3R)-3-methoxycyclohexyl)oxy)-4-(3-methoxypyridin-2-yl)-2-oxo-2H-pyran-6-carboxamide (compound 113A*), N-(5-(5-amino-1H-pyrazol-1-yl)-1,3,4-thiadiazol-2-yl)-3-(((1S,3S)-3-methoxycyclohexyl)oxy)-4-(3-methoxypyridin-2-yl)-2-oxo-2H-pyran-6-carboxamide (compound 113B*), N-(5-(5-amino-1H-pyrazol-1-yl)-1,3,4-thiadiazol-2-yl)-3-(((1S,3R)-3-methoxycyclohexyl)oxy)-4-(3-methoxypyridin-2-yl)-2-oxo-2H-pyran-6-carboxamide (compound 113C*) and N-(5-(5-amino-1H-pyrazol-1-yl)-1,3,4-thiadiazol-2-yl)-3-(((1R,3S)-3-methoxycyclohexyl)oxy)-4-(3-methoxypyridin-2-yl)-2-oxo-2H-pyran-6-carboxamide (compound 113D*)

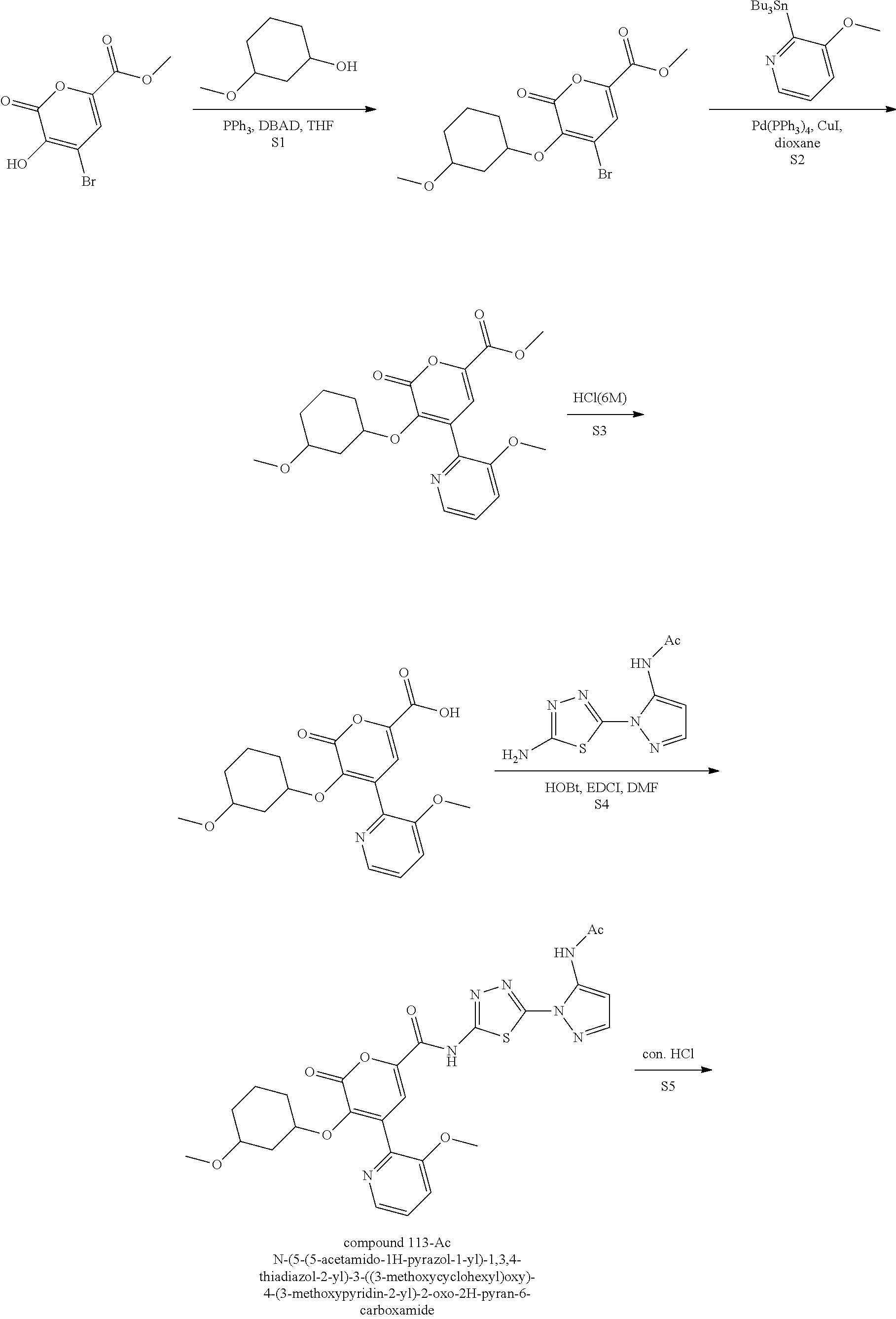

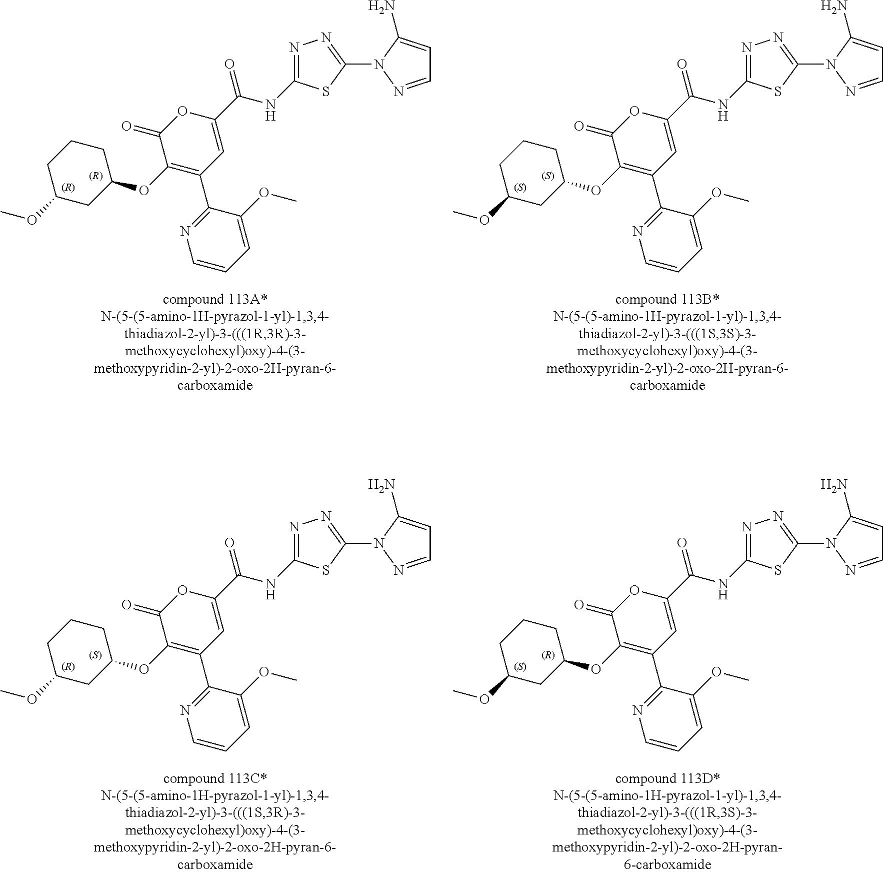

Step 1: To a stirred solution of methyl 4-bromo-5-hydroxy-6-oxopyran-2-carboxylate (1.00 g, 4.02 mmol, 1.0 equiv), 3-methoxycyclohexan-1-ol (626 mg, 4.81 mmol, 1.2 equiv) and triphenylphosphine (1.58 g, 6.02 mmol, 1.5 equiv) in tetrahydrofuran (THF) (10 mL) was added di-tert-butyl azodicarboxylate (DBAD) (1.38 g, 6.02 mmol, 1.5 equiv) at room temperature. The resulting mixture was stirred overnight at room temperature. The reaction was quenched with water (80 ml) at room temperature, extracted with ethyl acetate (3×80 mL). The combined organic layers were dried over anhydrous Na₂SO₄. After filtration, the filtrate was concentrated under reduced pressure. The residue was purified by silica gel column chromatography, eluted with petroleum ether (PE)/ethyl acetate (EA) (8:1) to afford methyl 4-bromo-5-[(3-methoxycyclohexyl)oxy]-6-oxopyran-2-carboxylate (1.8 g, 62% yield). LCMS (ES, m/z): 361.0 [M+H]J.

Step 2: To a solution of 4-bromo-5-[(3-methoxycyclohexyl)oxy]-6-oxopyran-2-carboxylate (1.50 g, 2.49 mmol, 1.0 equiv, 60% purity), tetrakis(triphenylphosphine)palladium(0) (Pd(PPh₃)₄) (576 mg, 0.498 mmol, 0.20 equiv) and copper(I) iodide (95 mg, 0.50 mmol, 0.20 equiv) in dioxane (40 mL) was added 3-methoxy-2-(tributylstannyl)pyridine (993 mg, 2.49 mmol, 1.0 equiv) dropwise at room temperature under vacuum, and then the resulting solution was stirred for 2 h at 100° C. under nitrogen atmosphere. The reaction was quenched with sat. KF (aq.) at room temperature, extracted with ethyl acetate (3×200 mL). The combined organic layers were dried over anhydrous Na₂SO₄. After filtration, the filtrate was concentrated under reduced pressure. The residue was purified by silica gel column chromatography, eluted with petroleum ether (PE)/ethyl acetate (EA) (10:1) to afford methyl 5-[(3-methoxycyclohexyl)oxy]-4-(3-methoxypyridin-2-yl)-6-oxopyran-2-carboxylate (700 mg, 65% yield). LCMS (ES, m/z): 390.1 [M+H]⁺.

Step 3: A solution of methyl 5-[(3-methoxycyclohexyl)oxy]-4-(3-methoxypyridin-2-yl)-6-oxopyran-2-carboxylate (1.10 g, 2.82 mmol, 1.0 equiv) in HCl (30 mL, 6M) was stirred for 3 h at 80° C. The resulting mixture was concentrated under reduced pressure to afford 5-[(3-methoxycyclohexyl)oxy]-4-(3-methoxypyridin-2-yl)-6-oxopyran-2-carboxylic acid (900 mg, 85% yield). LCMS (ES, m/z): 376.1 [M+H]⁺.

Step 4: To a solution of 5-[(3-methoxycyclohexyl)oxy]-4-(3-methoxypyridin-2-yl)-6-oxopyran-2-carboxylic acid (1.00 g, 2.66 mmol, 1.0 equiv), hydroxybenzotriazole (HOBT) (540 mg, 4.00 mmol, 1.5 equiv) and 1-ethyl-3-(3-dimethylaminopropyl)carbodiimide (EDCI) (1.02 g, 5.33 mmol, 2.0 equiv) in N,N-dimethylformamide (DMF) (20 mL) was added N-[2-(5-amino-1,3,4-thiadiazol-2-yl)pyrazol-3-yl]acetamide (478 mg, 2.13 mmol, 0.80 equiv) in portions at room temperature, and then the resulting solution was stirred for 1 h at room temperature. The reaction was quenched by the addition of water (100 mL) at room temperature, And then the precipitated solids were collected by filtration and washed with water (3×10 mL), and then the solid was concentrated under vacuum to afford N-[5-(5-acetamidopyrazol-1-yl)-1,3,4-thiadiazol-2-yl]-5-[(3-methoxycyclohexyl)oxy]-4-(3-methoxypyridin-2-yl)-6-oxopyran-2-carboxamide (Compound 113-Ac) (750 mg, 48% yield). LCMS (ES, m/z): 582.1 [M+H]⁺.

Step 5: A solution of N-[5-(5-acetamidopyrazol-1-yl)-1,3,4-thiadiazol-2-yl]-5-[(3-methoxycyclohexyl)oxy]-4-(3-methoxypyridin-2-yl)-6-oxopyran-2-carboxamide (Compound 113-Ac) (700 mg, 1.20 mmol, 1.0 equiv) in concentrated HCl (14 mL) was stirred for 4 h at room temperature. The resulting mixture was concentrated under reduced pressure. The residue was dissolved in dimethylsulfoxide (DMSO) (5 mL) and then poured into water (80 mL) and stirred for 30 min at room temperature, and then the precipitated solids were collected by filtration and washed with water (3×5 mL), and then the solid was dried under vacuum to afford (500 mg) as the mixture of 4 isomers, and then the mixture was separated by Prep-chiral-HPLC with the following conditions (Column: CHIRALPAK IE, 2*25 cm, 5 μm; Mobile Phase A: Hexanes (0.1% TFA), mobile phase B: ethanol:dichloromethane=1:1; flow rate: 20 mL/min; gradient: 80% B to 80% B in 22 min; wavelengths: 220/254 nm; afforded N-(5-(5-amino-1H-pyrazol-1-yl)-1,3,4-thiadiazol-2-yl)-3-(((1R,3R)-3-methoxycyclohexyl)oxy)-4-(3-methoxypyridin-2-yl)-2-oxo-2H-pyran-6-carboxamide (Compound 113A*; RT(min): 11.68) as the first eluting peak; N-(5-(5-amino-1H-pyrazol-1-yl)-1,3,4-thiadiazol-2-yl)-3-(((1S,3S)-3-methoxycyclohexyl)oxy)-4-(3-methoxypyridin-2-yl)-2-oxo-2H-pyran-6-carboxamide (Compound 113B*; RT(min): 13.86) as the second eluting peak; and a mixture of N-(5-(5-amino-1H-pyrazol-1-yl)-1,3,4-thiadiazol-2-yl)-3-(((IS,3R)-3-methoxycyclohexyl)oxy)-4-(3-methoxypyridin-2-yl)-2-oxo-2H-pyran-6-carboxamide (Compounds 113C*) and N-(5-(5-amino-1H-pyrazol-1-yl)-1,3,4-thiadiazol-2-yl)-3-(((1R,3S)-3-methoxycyclohexyl)oxy)-4-(3-methoxypyridin-2-yl)-2-oxo-2H-pyran-6-carboxamide (Compound 113D*) as the third eluting peak (RT(min): 16.20). The mixture of Compounds 113C* and 113D* (300 mg) was further purified by prep-chiral-HPLC (CHIRAL ART Cellulose-SC, 2*25 cm, 5 μm; mobile phase A: hexanes (0.1% TFA), mobile phase B: (ethanol:dichloromethane=1:1); flow rate: 20 mL/min; gradient: isocratic 45; wavelengths: 220/254 nm) to provide Compound 113C* as the first eluting peak (RT(min): 11.48), and Compound 113D* as the second eluting peak (RT(min): 13.73). *Stereochemistry arbitrarily assigned.

Compound 113A*: 74.2 mg, 11% yield. LCMS (ES, m/z): 540.1 [M+H]+; 1H NMR (400 MHz, DMSO-d6) δ 13.35 (s, 1H), 8.31 (d, J=4.4 Hz, 1H), 7.66 (d, J=8.4 Hz, 1H), 7.54 (dd, J=8.4, 4.4 Hz, 1H), 7.47-7.41 (m, 2H), 6.65 (s, 2H), 5.43 (d, J=1.6 Hz, 1H), 4.73-4.69 (m, 1H), 3.86 (s, 3H), 3.05 (s, 3H), 2.93-2.89 (m, 1H), 1.69-1.65 (m, 1H), 1.55-1.30 (m, 5H), 114-1.07 (m, 2H).

Compound 113B*: 40.7 mg, 6% yield. LCMS (ES, m/z): 540.1[M+H]+; 1H NMR (400 MHz, DMSO-d6) δ 13.32 (s, 1H), 8.30 (d, J=4.8 Hz, 1H), 7.65 (d, J=8.4 Hz, 1H), 7.53-7.48 (m, 3H), 6.66 (s, 2H), 5.44 (d, J=1.6 Hz, 1H), 4.32-4.26 (m, 1H), 3.85 (s, 3H), 3.16 (s, 3H), 3.03-2.98 (m, 1H), 2.21-2.17 (m, 1H), 1.85-1.79 (m, 2H), 1.63-1.59 (m, 1H), 1.10-1.02 (m, 1H), 0.93-0.77 (m, 3H).

Compound 113C*: 34.0 mg, 5% yield. LCMS (ES, m/z): 540.1 [M+H]+; 1H NMR (400 MHz, DMSO-d6) δ13.35 (s, 1H), 8.31 (d, J=4.4 Hz, 1H), 7.66 (d, J=8.4 Hz, 1H), 7.54 (dd. J=8.4, 4.4 Hz, 1H), 7.47-7.41 (m, 2H), 6.65 (s, 2H), 5.43 (d, J=1.6 Hz, 1H), 4.73-4.69 (m, 1H), 3.86 (s, 3H), 3.05 (s, 3H), 2.93-2.89 (m, 1H), 1.69-1.65 (m, 1H), 1.55-1.30 (m, 5H), 114-1.07 (m, 2H).

Compound 113D*: 31.9 mg, 5% yield. LCMS (ES, m/z): 540.1 [M+H]+; 1H NMR (400 MHz, DMSO-d6) δ 13.32 (s, 1H), 8.30 (d, J=4.8 Hz, 1H), 7.65 (d, J=8.4 Hz, 1H), 7.53-7.48 (m, 3H), 6.66 (s, 2H), 5.44 (d, J=1.6 Hz, 1H), 4.32-4.26 (m, 1H), 3.85 (s, 3H), 3.16 (s, 3H), 3.03-2.98 (m, 1H), 2.21-2.17 (m, 1H), 1.85-1.79 (m, 2H), 1.63-1.59 (m, 1H), 1.10-1.02 (m, 1H), 0.93-0.77 (m, 3H).

Compounds provided in below Table A were synthesized, or may be synthesized, following the Procedure as described, with reference to the Examples. LC-MS data is provided for each compound synthesized. Dashed lines (--) indicate no data available. The Asterix (*) next to the Compound Number (#) signifies that arbitrary stereochemistry is assigned. “Rac-X” signifies a mixture of 2 or more stereoisomers.

TABLE A
Additional Compounds

| | | LCMS | |
|---|---|---|---|
| # | Name | [M + 1]+ | Procedure |
| 2 | 3-methoxy-N-(5-(5-methyl-1H- | 488.05 | Compound 2 was prepared |
| | pyrazol-1-yl)-1,3,4-thiadiazol-2- | | according to Example 1, Step 10 |
| | yl)-4-(2-(methylsulfonyl)phenyl)- | | using (2- |
| | 2-oxo-2H-pyran-6-carboxamide | | (methylsulfonyl)phenyl)boronic |
| | | | acid as the “aryl boron reagent”, |
| | | | Cs₂CO₃ in place of K₂CO₃ as the |
| | | | base, and dioxane/H₂O in place of |
| | | | DMF as solvent |
| 11 | N-(5-(5-amino-1H-pyrazol-1-yl)- | 499.15 | Compound 11 was prepared |
| | 1,3,4-thiadiazol-2-yl)-4-(2- | | according to Example 7 using (2- |
| | methoxy-6-methylphenyl)-3-(2- | | methoxy-6-methylphenyl)boronic |
| | methoxyethoxy)-2-oxo-2H-pyran- | | acid in place of 3-methoxy-2- |
| | 6-carboxamide | | (4,4,5,5-tetramethyl-1,3,2- |
| | | | dioxaborolan-2-yl)benzonitrile |
| 13 | N-(5-(5-amino-1H-pyrazol-1-yl)- | 503.2 | Compound 13 was prepared |
| | 1,3,4-thiadiazol-2-yl)-4-(2-fluoro- | | according to Example 5 Steps 3-4 |
| | 6-methoxyphenyl)-3-(2- | | using 2-fluoro-6- |
| | methoxyethoxy)-2-oxo-2H-pyran- | | methoxyphenylboronic acid in |
| | 6-carboxamide | | place of 2,6- |
| | | | dimethoxyphenylboronic acid |
| 30 | N-(5-(5-amino-1H-pyrazol-1-yl)- | 557.1 | Compound 30 was prepared |
| | 1,3,4-thiadiazol-2-yl)-4-(2,6- | | according to Example 16 using |
| | dimethoxyphenyl)-3-(2-(2- | | diglycol methyl ether in place of |
| | methoxyethoxy)ethoxy)-2-oxo-2H- | | 1,3-dimethoxypropan-2-ol |
| | pyran-6-carboxamide | | |
| 41 | N-(5-(5-amino-1H-pyrazol-1-yl)- | 505.1 | Compound 41 was prepared |
| | 1,3,4-thiadiazol-2-yl)-4-(2-chloro- | | according to Example 5 Steps 5-6 |
| | 6-hydroxyphenyl)-3-(2- | | using N-[5-(5-acetamidopyrazol-1- |
| | methoxyethoxy)-2-oxo-2H-pyran- | | yl)-1,3,4-thiadiazol-2-yl]-4-iodo-5- |
| | 6-carboxamide | | (2-methoxyethoxy)-6-oxopyran-2- |
| | | | carboxamide in place of 4-bromo- |
| | | | N-[5-(5-acetamidopyrazol-1-yl)- |
| | | | 1,3,4-thiadiazol-2-yl]-5-(2- |
| | | | methoxyethoxy)-6-oxopyran-2- |
| | | | carboxamide and 3-chloro-2- |
| | | | (4,4,5,5-tetramethyl-1,3,2- |
| | | | dioxaborolan-2-yl)phenol in place |
| | | | of 2,6-dimethoxyphenylboronic |
| | | | acid |
| 42 | N-(5-(5-amino-1H-pyrazol-1-yl)- | 541.2 | Compound 42 was prepared |
| | 1,3,4-thiadiazol-2-yl)-4-(2,6- | | according to Example 16 using (1- |
| | dimethoxyphenyl)-3-((1- | | methoxycyclopropyl)methanol in |
| | methoxycyclopropyl)methoxy)-2- | | place of 1,3-dimethoxypropan-2-ol |
| | oxo-2H-pyran-6-carboxamide | | and methyl 4-bromo-5-hydroxy-6- |
| | | | oxopyran-2-carboxylate in place of |
| | | | methyl 4-iodo-5-hydroxy-6- |
| | | | oxopyran-2-carboxylate |
| 49 | N-(5-(5-amino-1H-pyrazol-1-yl)- | 516.1 | Compound 49 was prepared |
| | 1,3,4-thiadiazol-2-yl)-4-(3,6- | | according to Example 15 using 2- |
| | dimethoxypyridin-2-yl)-3-(2- | | bromo-3,6-dimethoxypyridine in |
| | methoxyethoxy)-2-oxo-2H-pyran- | | place of 3-bromo-2,4- |
| | 6-carboxamide | | dimethoxypyridine |
| 53 | (S)-N-(5-(5-amino-1H-pyrazol-1- | 529.1 | Compound 53 was prepared |
| | yl)-1,3,4-thiadiazol-2-yl)-4-(2,6- | | according to Example 35 using |
| | dimethoxyphenyl)-3-(2- | | (2S)-2-methoxypropan-1-ol in |
| | methoxypropoxy)-2-oxo-2H- | | place of (2R)-2-(benzyloxy) |
| | pyran-6-carboxamide | | propan-1-ol and methyl 4-bromo-5- |
| | | | hydroxy-6-oxopyran-2-carboxylate |
| | | | in place of methyl 4-iodo-5- |
| | | | hydroxy-6-oxopyran-2-carboxylate |
| 54 | (R)-N-(5-(5-amino-1H-pyrazol-1- | 529.1 | Compound 54 was prepared |
| | yl)-1,3,4-thiadiazol-2-yl)-4-(2,6- | | according to Example 35 using (R)- |
| | dimethoxyphenyl)-3-(2- | | 2-methoxypropan-1-ol in place of |
| | methoxypropoxy)-2-oxo-2H- | | (2R)-2-(benzyloxy) propan-1-ol |
| | pyran-6-carboxamide | | and methyl 4-bromo-5-hydroxy-6- |
| | | | oxopyran-2-carboxylate in place of |
| | | | methyl 4-iodo-5-hydroxy-6- |
| | | | oxopyran-2-carboxylate |
| 58 | N-(5-(5-amino-1H-pyrazol-1-yl)- | 520.1 | Compound 58 was prepared |
| | 1,3,4-thiadiazol-2-yl)-4-(5-chloro- | | according to Example 15 using 2- |
| | 3-methoxypyridin-2-yl)-3-(2- | | bromo-5-chloro-3-methoxypyridine |
| | methoxyethoxy)-2-oxo-2H-pyran- | | in place of 3-bromo-2,4- |
| | 6-carboxamide | | dimethoxypyridine |
| 61 | N-(5-(5-amino-1H-pyrazol-1-yl)- | 522.1 | Compound 61 was prepared |
| | 1,3,4-thiadiazol-2-yl)-3-(2- | | according to Example 24 using 2- |
| | (difluoromethoxy)ethoxy)-4-(3- | | (difluoromethoxy)ethanol in place |
| | methoxypyridin-2-yl)-2-oxo-2H- | | of 3-methoxy-2,2-dimethylpropan- |
| | pyran-6-carboxamide | | 1-ol, DIAD in place of DBAD, and |
| | | | methyl 4-bromo-5-hydroxy-6- |
| | | | oxopyran-2-carboxylate in place of |
| | | | methyl 4-iodo-5-hydroxy-6- |
| | | | oxopyran-2-carboxylate |
| 66 | N-(5-(5-amino-1H-pyrazol-1-yl)- | 511.0 | Compound 66 was prepared |
| | 1,3,4-thiadiazol-2-yl)-4-(4-cyano- | | according to Example 42 using 3- |
| | 2-methoxypyridin-3-yl)-3-(2- | | bromo-2-methoxypyridine-4- |
| | methoxyethoxy)-2-oxo-2H-pyran- | | carbonitrile in place of 2-bromo- |
| | 6-carboxamide | | 3,5-dimethoxypyridine |
| 69 | N-(5-(5-amino-1H-pyrazol-1-yl)- | 568.2 | Compound 69 was prepared |
| | 1,3,4-thiadiazol-2-yl)-4-(2,6- | | according to Example 36 using 1- |
| | dimethoxyphenyl)-2-oxo-3-(2- | | piperidineethanol in place of (2R)- |
| | (piperidin-1-yl)ethoxy)-2H-pyran- | | 1-methoxypropan-2-ol |
| | 6-carboxamide | | |
| 70 | N-(5-(5-amino-1H-pyrazol-1-yl)- | 512.1 | Compound 70 was prepared |
| | 1,3,4-thiadiazol-2-yl)-4-(3- | | according to Example 42 using 2- |
| | cyclopropoxypyridin-2-yl)-3-(2- | | bromo-3-cyclopropoxypyridine in |
| | methoxyethoxy)-2-oxo-2H-pyran- | | place of 2-bromo-3,5- |
| | 6-carboxamide | | dimethoxypyridine |
| 38A* | (S)-N-(5-(5-amino-1H-pyrazol-1- | 545.1 | Compounds 38A* and 38B* were |
| | yl)-1,3,4-thiadiazol-2-yl)-4-(2,6- | | prepared according to Example 43 |
| | dimethoxyphenyl)-3-(3-hydroxy-2- | | using 2-methoxypropane-1,3-diol |
| | methoxypropoxy)-2-oxo-2H- | | in place of glycerin methyl ether; |
| | pyran-6-carboxamide | | followed by chiral HPLC |
| 38B* | (R)-N-(5-(5-amino-1H-pyrazol-1- | 545.4 | separation. |
| | yl)-1,3,4-thiadiazol-2-yl)-4-(2,6- | | |
| | dimethoxyphenyl)-3-(3-hydroxy-2- | | |
| | methoxypropoxy)-2-oxo-2H- | | |
| | pyran-6-carboxamide | | |
| 44 | N-(5-(5-amino-1H-pyrazol-1-yl)- | 586.2 | Compound 44 was prepared |
| | 1,3,4-thiadiazol-2-yl)-4-(2,6- | | according to Example 18 using 4- |
| | dimethoxyphenyl)-2-oxo-3-(2- | | thiomorpholineethanol in place of |
| | thiomorpholinoethoxy)-2H-pyran- | | 4-morpholineethanol. |
| | 6-carboxamide | | |
| 52A | (S)-N-(5-(5-amino-1H-pyrazol-1- | 527.2 | Compound 52A was prepared |
| | yl)-1,3,4-thiadiazol-2-yl)-4-(2,6- | | according to Example 46 using |
| | dimethoxyphenyl)-2-oxo-3- | | (3R)-oxolan-3-ol in place of (1,2- |
| | ((tetrahydrofuran-3-yl)oxy)-2H- | | cis)-cyclopentane-1,2-diol. The |
| | pyran-6-carboxamide | | compound was purified by |
| | | | reversed-phase chromatography |
| | | | using the following conditions: |
| | | | Column, C18 silica gel; mobile |
| | | | phase, MeCN in Water, 20% to |
| | | | 40% gradient in 10 min; detector, |
| | | | UV 254 nm. |
| 52B | (R)-N-(5-(5-amino-1H-pyrazol-1- | 527.1 | Compound 52B was prepared |
| | yl)-1,3,4-thiadiazol-2-yl)-4-(2,6- | | according to Example 46 using |
| | dimethoxyphenyl)-2-oxo-3- | | (3S)-oxolan-3-ol in place of (1,2- |
| | ((tetrahydrofuran-3-yl)oxy)-2H- | | cis)-cyclopentane-1,2-diol. The |
| | pyran-6-carboxamide | | compound was purified by |
| | | | reversed-phase chromatography |
| | | | using the following conditions: |
| | | | Column, C18 silica gel; mobile |
| | | | phase, MeCN in Water, 50% to |
| | | | 60% gradient in 10 min; detector, |
| | | | UV 254 nm. |
| 56A | (S)-N-(5-(5-amino-1H-pyrazol-1- | 541.1 | Compound 56A was prepared |
| | yl)-1,3,4-thiadiazol-2-yl)-4-(2,6- | | according to Example 33 using |
| | dimethoxyphenyl)-2-oxo-3- | | (2S)-oxolan-2-ylmethanol in place |
| | ((tetrahydrofuran-2-yl)methoxy)- | | of 1,4-dioxan-2-ylmethanol. |
| | 2H-pyran-6-carboxamide | | Compound 56A was purified by |
| | | | reversed-phase Prep-HPLC with |
| | | | the following conditions: Column: |
| | | | XBridge Prep OBD C18 Column, |
| | | | 30*150 mm, 5 μm; Mobile Phase A: |
| | | | Water(10 mmol/L NH₄HCO₃), |
| | | | Mobile Phase B: ACN; Flow rate: |
| | | | 60 mL/min; Gradient: 18% B to |
| | | | 28% B in 7.8 min; Wave Length: |
| | | | 254/252 nm; RT(min): 12.25. |
| 56B | (R)-N-(5-(5-amino-1H-pyrazol-1- | 541.2 | Compound 56B was prepared |
| | yl)-1,3,4-thiadiazol-2-yl)-4-(2,6- | | according to Example 33 using |
| | dimethoxyphenyl)-2-oxo-3- | | (2R)-oxolan-2-ylmethanol in place |
| | ((tetrahydrofuran-2-yl)methoxy)- | | of 1,4-dioxan-2-ylmethanol. |
| | 2H-pyran-6-carboxamide | | Compound 56B was purified by |
| | | | Prep-HPLC with the following |
| | | | conditions (Column: XBridge Prep |
| | | | Phenyl OBD Column19*250 mm; |
| | | | Mobile Phase A: |
| | | | Water(0.05% TFA), Mobile Phase |
| | | | B: acetonitrile; Flow rate: 25 |
| | | | mL/min; Gradient: 40% B to 50% |
| | | | B in 10 min; Wave Length: |
| | | | 254/220 nm; RT(min): 13) |
| 57A | N-(5-(5-amino-1H-pyrazol-1-yl)- | 527.1 | Compound 57A was prepared |
| | 1,3,4-thiadiazol-2-yl)-4-(2,6- | | according to Example 33 using |
| | dimethoxyphenyl)-3-((1,3-trans)-3- | | (1,3-cis)-cyclobutane-1,3-diol in |
| | hydroxycyclobutoxy)-2-oxo-2H- | | place of 1,4-dioxan-2-ylmethanol. |
| | pyran-6-carboxamide | | Compound 57A was purified by |
| | | | reversed-phase flash |
| | | | chromatography with the following |
| | | | conditions: column, C18 silica gel; |
| | | | mobile phase, MeCN in Water |
| | | | (0.1% TFA), 10% to 50% gradient |
| | | | in 20 min RT:(15 min); detector, |
| | | | UV 254 nm. |
| 57B | N-(5-(5-amino-1H-pyrazol-1-yl)- | 527.1 | Compound 57B was prepared |
| | 1,3,4-thiadiazol-2-yl)-4-(2,6- | | according to Example 33 using |
| | dimethoxyphenyl)-3-((1,3-cis)-3- | | (1,3-trans)-cyclobutane-1,3-diol in |
| | hydroxycyclobutoxy)-2-oxo-2H- | | place of 1,4-dioxan-2-ylmethanol. |
| | pyran-6-carboxamide | | |
| 64A* | N-(5-(5-amino-1H-pyrazol-1-yl)- | 541.1 | Compounds 64A* and 64B* were |
| | 1,3,4-thiadiazol-2-yl)-4-(2,6- | | prepared according to Example 45 |
| | dimethoxyphenyl)-3-(((1R,3R)-3- | | using (1,3-cis)-cyclopentane-1,3- |
| | hydroxycyclopentyl)oxy)-2-oxo- | | diol in place of 2-{2-[(tert- |
| | 2H-pyran-6-carboxamide | | butyldimethylsilyl)oxy]ethoxy}ethanol. |
| 64B* | N-(5-(5-amino-1H-pyrazol-1-yl)- | | Compound 64A was purified |
| | 1,3,4-thiadiazol-2-yl)-4-(2,6- | | by reversed-phase flash |
| | dimethoxyphenyl)-3-(((1S,3S)-3- | | chromatography with the following |
| | hydroxycyclopentyl)oxy)-2-oxo- | | conditions: column, C18 silica gel; |
| | 2H-pyran-6-carboxamide | | mobile phase, MeCN in Water, |
| | | | 40% to 60% gradient in 10 min; |
| | | | detector, UV 254 nm. |
| 64C* | N-(5-(5-amino-1H-pyrazol-1-yl)- | 541.3 | Compounds 64C* and 64D* were |
| | 1,3,4-thiadiazol-2-yl)-4-(2,6- | | prepared according to Example 45 |
| | dimethoxyphenyl)-3-(((1R,3S)-3- | | using (1,3-trans)-cyclopentane-1,3- |
| | hydroxycyclopentyl)oxy)-2-oxo- | | diol in place of 2-{2-[(tert- |
| | 2H-pyran-6-carboxamide | | butyldimethylsilyl)oxy]ethoxy}ethanol. |
| 64D* | N-(5-(5-amino-1H-pyrazol-1-yl)- | | |
| | 1,3,4-thiadiazol-2-yl)-4-(2,6- | | |
| | dimethoxyphenyl)-3-(((1S,3R)-3- | | |
| | hydroxycyclopentyl)oxy)-2-oxo- | | |
| | 2H-pyran-6-carboxamide | | |
| 65 | 3-(2-(2-thia-6-azaspiro[3.3]heptan- | 598.2 | Compound 65 was prepared |
| | 6-yl)ethoxy)-N-(5-(5-amino-1H- | | according to Example 33 using 2- |
| | pyrazol-1-yl)-1,3,4-thiadiazol-2- | | (t-butoxy)ethanol in place of 1,4- |
| | yl)-4-(2,6-dimethoxyphenyl)-2- | | dioxan-2-ylmethanol in Step 1. |
| | oxo-2H-pyran-6-carboxamide | | Then, before engaging the material |
| | | | in step 3, additional manipulation |
| | | | was followed: t-Bu deprotection |
| | | | using HCl (see Example 13, step 6) |
| | | | followed by mesylate formation |
| | | | (see Example 26, step 2) and SN₂ |
| | | | reaction using 2-thia-6- |
| | | | azaspiro[3.3]heptane hemioxalate |
| | | | and K₂CO₃ in MeCN. |
| 72 | 3-(2-(1-thia-6-azaspiro[3.3]heptan- | 598.2 | Compound 72 was prepared |
| | 6-yl)ethoxy)-N-(5-(5-amino-1H- | | according to Example 33 using 2- |
| | pyrazol-1-yl)-1,3,4-thiadiazol-2- | | (t-butoxy)ethanol in place of 1,4- |
| | yl)-4-(2,6-dimethoxyphenyl)-2- | | dioxan-2-ylmethanol in Step 1. |
| | oxo-2H-pyran-6-carboxamide | | Then, before engaging the material |
| | | | in step 3, the additional steps were |
| | | | taken: t-Bu deprotection using HCl |
| | | | (see Example 13, step 6) followed |
| | | | by mesylate formation (see |
| | | | Example 26, step 2) and SN₂ |
| | | | reaction using 1-Thia-6- |
| | | | azaspiro[3.3]heptane hydrochloride |
| | | | and K₂CO₃ in MeCN. |
| 75 | N-(5-(5-amino-1H-pyrazol-1-yl)- | 600.2 | Compound 75 was prepared |
| | 1,3,4-thiadiazol-2-yl)-4-(2,6- | | according to Example 33 using 4- |
| | dimethoxyphenyl)-2-oxo-3-(2-(3- | | (2-hydroxyethyl)thiomorpholin-3- |
| | oxothiomorpholino)ethoxy)-2H- | | one in place of 1,4-dioxan-2- |
| | pyran-6-carboxamide | | ylmethanol. |
| 76A* | (R)-N-(5-(5-amino-1H-pyrazol-1- | — | Compounds 76A* and 76B* may |
| | yl)-1,3,4-thiadiazol-2-yl)-4-(2,6- | | be prepared according to Example |
| | dimethoxyphenyl)-3-(2-hydroxy-2- | | 33 using 1-(tetrahydro-2H- |
| | (tetrahydro-2H-thiopyran-4- | | thiopyran-4-yl)ethane-1,2-diol (see |
| | yl)ethoxy)-2-oxo-2H-pyran-6- | | J. Am. Chem. Soc. 1954, 76, 5703- |
| | carboxamide | | 5705 for its preparation) in place of |
| 76B* | (S)-N-(5-(5-amino-1H-pyrazol-1- | — | 1,4-dioxan-2-ylmethanol; followed |
| | yl)-1,3,4-thiadiazol-2-yl)-4-(2,6- | | by chiral HPLC separation. |
| | dimethoxyphenyl)-3-(2-hydroxy-2- | | |
| | (tetrahydro-2H-thiopyran-4- | | |
| | yl)ethoxy)-2-oxo-2H-pyran-6- | | |
| | carboxamide | | |
| 77A* | (R)-N-(5-(5-amino-1H-pyrazol-1- | — | Compounds 77A* and 77B* may |
| | yl)-1,3,4-thiadiazol-2-yl)-4-(2,6- | | be prepared according to Example |
| | dimethoxyphenyl)-3-(2-hydroxy-2- | | 33 using 1-(thietan-3-yl)ethane-1,2- |
| | (thietan-3-yl)ethoxy)-2-oxo-2H- | | diol in place of 1,4-dioxan-2- |
| | pyran-6-carboxamide | | ylmethanol; followed by chiral |
| | | | HPLC separation. 1-(thietan-3- |
| 77B* | (S)-N-(5-(5-amino-1H-pyrazol-1- | — | yl)ethane-1,2-diol can be made |
| | yl)-1,3,4-thiadiazol-2-yl)-4-(2,6- | | using similar conditions as |
| | dimethoxyphenyl)-3-(2-hydroxy-2- | | described in J. Am. Chem. Soc. |
| | (thietan-3-yl)ethoxy)-2-oxo-2H- | | 1954, 76, 5703-5705 starting from |
| | pyran-6-carboxamide | | thietane-3-carboxylic acid. |
| 78A* | (S)-N-(5-(5-amino-1H-pyrazol-1- | 630.1 | Compounds 78A* and 78B* were |
| | yl)-1,3,4-thiadiazol-2-yl)-4-(2,6- | | prepared using methyl 5-hydroxy- |
| | dimethoxyphenyl)-3-((1-methoxy- | | 4-bromo-6-oxopyran-2-carboxylate |
| | 3-thiomorpholinopropan-2-yl)oxy)- | | (instead of methyl 5-hydroxy-4- |
| | 2-oxo-2H-pyran-6-carboxamide | | iodo-6-oxopyran-2-carboxylate) |
| 78B* | (R)-N-(5-(5-amino-1H-pyrazol-1- | | and 5-({1-[(tert- |
| | yl)-1,3,4-thiadiazol-2-yl)-4-(2,6- | | butyldiphenylsilyl)oxy]-3- |
| | dimethoxyphenyl)-3-((1-methoxy- | | methoxypropan-2-yl}oxy)-4-(2,6- |
| | 3-thiomorpholinopropan-2-yl)oxy)- | | dimethoxyphenyl)-N-[5-(5- |
| | 2-oxo-2H-pyran-6-carboxamide | | acetamidopyrazol-1-yl)-1,3,4- |
| | | | thiadiazol-2-yl]-6-oxopyran-2- |
| | | | carboxamide (made according to |
| | | | Example 43, steps 1-5), then |
| | | | performing a silyl deprotection |
| | | | using TBAF, a mesylation (see |
| | | | Example 26, step 2), followed by |
| | | | SN₂ reaction using thiomorpholine |
| | | | and acetate deprotection (see |
| | | | Example 43, step 6); followed by |
| | | | chiral HPLC separation. |
| 79A* | (R)-N-(5-(5-amino-1H-pyrazol-1- | 616.0 | Compounds 79A* and 79B* were |
| | yl)-1,3,4-thiadiazol-2-yl)-4-(2,6- | | prepared according to steps 2-6 of |
| | dimethoxyphenyl)-3-(3-hydroxy-2- | | Example 43, but using methyl 5- |
| | thiomorpholinopropoxy)-2-oxo- | | hydroxy-4-bromo-6-oxopyran-2- |
| | 2H-pyran-6-carboxamide | | carboxylate and 2- |
| 79B* | (S)-N-(5-(5-amino-1H-pyrazol-1- | 616.1 | thiomorpholinopropane-1,3-diol in |
| | yl)-1,3,4-thiadiazol-2-yl)-4-(2,6- | | Step 2 instead of methyl 5- |
| | dimethoxyphenyl)-3-(3-hydroxy-2- | | hydroxy-4-iodo-6-oxopyran-2- |
| | thiomorpholinopropoxy)-2-oxo- | | carboxylate and tert-butyl(2- |
| | 2H-pyran-6-carboxamide | | hydroxy-3- |
| | | | methoxypropoxy)diphenylsilane. |
| | | | 2-Thiomorpholinopropane-1,3-diol |
| | | | was prepared starting from 3- |
| | | | (benzyloxy)propane-1,2-diol using |
| | | | a modified silyl protection |
| | | | described in step 1 of Example 43 |
| | | | (Et₃N and DMAP replacing |
| | | | imidazole), followed by |
| | | | mesylation, SN₂ reaction using |
| | | | thiomorpholine as the nucleophile |
| | | | and deprotection of the silyl group |
| | | | using TBAF followed by chiral |
| | | | HPLC separation. |
| 80A* | (R)-N-(5-(5-amino-1H-pyrazol-1- | — | Compounds 80A* and 80B* may |
| | yl)-1,3,4-thiadiazol-2-yl)-4-(2,6- | | be prepared using N-(5-(5- |
| | dimethoxyphenyl)-3-(3-methoxy- | | acetamido-1H-pyrazol-1-yl)-1,3,4- |
| | 2-thiomorpholinopropoxy)-2-oxo- | | thiadiazol-2-yl)-3-(2-((tert- |
| | 2H-pyran-6-carboxamide | | butyldiphenylsilyl)oxy)-3- |
| 80B* | (S)-N-(5-(5-amino-1H-pyrazol-1- | — | methoxypropoxy)-4-(2,6- |
| | yl)-1,3,4-thiadiazol-2-yl)-4-(2,6- | | dimethoxyphenyl)-2-oxo-2H- |
| | dimethoxyphenyl)-3-(3-methoxy- | | pyran-6-carboxamide (made |
| | 2-thiomorpholinopropoxy)-2-oxo- | | according to Example 43, steps 1- |
| | 2H-pyran-6-carboxamide | | 5), then performing a mesylation |
| | | | (see Example 26, step 2), followed |
| | | | by SN₂ reaction using |
| | | | thiomorpholine and acetate |
| | | | deprotection (see Example 43, step |
| | | | 6); followed by chiral HPLC |
| | | | separation. |
| 81A* | (S)-N-(5-(5-amino-1H-pyrazol-1- | 617.2 | Compounds 81A* and 81* were |
| | yl)-1,3,4-thiadiazol-2-yl)-4-(2,6- | | prepared according to Example 33 |
| | dimethoxyphenyl)-3-((1-hydroxy- | | using 1-((tert- |
| | 3-thiomorpholinopropan-2-yl)oxy)- | | butyldiphenylsilyl)oxy)-3- |
| | 2-oxo-2H-pyran-6-carboxamide | | thiomorpholinopropan-2-ol in place |
| 81B* | (R)-N-(5-(5-amino-1H-pyrazol-1- | 616.2 | of 1,4-dioxan-2-ylmethanol; |
| | yl)-1,3,4-thiadiazol-2-yl)-4-(2,6- | | followed by chiral HPLC |
| | dimethoxyphenyl)-3-((1-hydroxy- | | separation. 1-((Tert- |
| | 3-thiomorpholinopropan-2-yl)oxy)- | | butyldiphenylsilyl)oxy)-3- |
| | 2-oxo-2H-pyran-6-carboxamide | | thiomorpholinopropan-2-ol was |
| | | | prepared in 2 steps by reacting |
| | | | glycidol with thiomorpholine and a |
| | | | base, followed by silyl protection |
| | | | (see step 1 of Example 37 using 3- |
| | | | (morpholin-4-yl)propane-1,2-diol |
| | | | in place of 2-bromo-3- |
| | | | (hydroxymethyl)benzonitrile)). |
| 82A* | (S)-N-(5-(5-amino-1H-pyrazol-1- | 630.2 | Compounds 82A* and 82B* were |
| | yl)-1,3,4-thiadiazol-2-yl)-4-(2,6- | | prepared following Example 33 |
| | dimethoxyphenyl)-3-(2-(2- | | using (S)-2- |
| | ((methylthio)methyl)morpholino)ethoxy)- | | ((methylthio)methyl)morpholine in |
| | 2-oxo-2H-pyran-6-carboxamide | | place of 1,4-dioxan-2-ylmethanol. |
| 82B* | (R)-N-(5-(5-amino-1H-pyrazol-1- | 630.2 | (S)-2-((methylthio)methyl)morpholine |
| | yl)-1,3,4-thiadiazol-2-yl)-4-(2,6- | | was from (S)-morpholin-2- |
| | dimethoxyphenyl)-3-(2-(2- | | ylmethanol in 4 steps: CBz |
| | ((methylthio)methyl)morpholino)ethoxy)- | | protection using CbzCl and a base, |
| | 2-oxo-2H-pyran-6-carboxamide | | followed by mesylation (see |
| | | | Example 26, step 2), SN₂ using |
| | | | sodium thiomethoxide and CBz |
| | | | deprotection in TFA at 50° C. |
| 83A* | (R)-3-((1,4-oxathian-2- | 573.0 | Compounds 83A* and 83B* were |
| | yl)methoxy)-N-(5-(5-amino-1H- | | prepared following Example 33 |
| | pyrazol-1-yl)-1,3,4-thiadiazol-2- | | using (1,4-oxathian-2-yl)methanol |
| | yl)-4-(2,6-dimethoxyphenyl)-2- | | in place of 1,4-dioxan-2- |
| | oxo-2H-pyran-6-carboxamide | | ylmethanol. |
| 83B* | (S)-3-((1,4-oxathian-2- | | |
| | yl)methoxy)-N-(5-(5-amino-1H- | | |
| | pyrazol-1-yl)-1,3,4-thiadiazol-2- | | |
| | yl)-4-(2,6-dimethoxyphenyl)-2- | | |
| | oxo-2H-pyran-6-carboxamide | | |
| 84A* | (S)-N-(5-(5-amino-1H-pyrazol-1- | 614.2 | Compounds 84A* and 84B* were |
| | yl)-1,3,4-thiadiazol-2-yl)-4-(2,6- | | prepared using methyl 5-hydroxy- |
| | dimethoxyphenyl)-3-((1-methoxy- | | 4-bromo-6-oxopyran-2-carboxylate |
| | 3-morpholinopropan-2-yl)oxy)-2- | | (instead of methyl 5-hydroxy-4- |
| | oxo-2H-pyran-6-carboxamide | | iodo-6-oxopyran-2-carboxylate) |
| 84B* | (R)-N-(5-(5-amino-1H-pyrazol-1- | | and 5-({1-[(tert- |
| | yl)-1,3,4-thiadiazol-2-yl)-4-(2,6- | | butyldiphenylsilyl)oxy]-3- |
| | dimethoxyphenyl)-3-((1-methoxy- | | methoxypropan-2-yl}oxy)-4-(2,6- |
| | 3-morpholinopropan-2-yl)oxy)-2- | | dimethoxyphenyl)-N-[5-(5- |
| | oxo-2H-pyran-6-carboxamide | | acetamidopyrazol-1-yl)-1,3,4- |
| | | | thiadiazol-2-yl]-6-oxopyran-2- |
| | | | carboxamide (made according to |
| | | | Example 43, steps 1-5), then |
| | | | performing a silyl deprotection |
| | | | using TBAF, a mesylation (see |
| | | | Example 26, step 2), followed by |
| | | | SN₂ reaction using morpholine and |
| | | | acetate deprotection (see Example |
| | | | 43, step 6); followed by chiral |
| | | | HPLC separation. |
| Rac-85 | N-(5-(5-amino-1H-pyrazol-1-yl)- | 600.2 | Compound 85 (comprising a |
| | 1,3,4-thiadiazol-2-yl)-4-(2,6- | | mixture of 85A* and 85B*) was |
| | dimethoxyphenyl)-3-(3-hydroxy-2- | | prepared according to steps 2-6 of |
| | morpholinopropoxy)-2-oxo-2H- | | Example 43, but using 2- |
| | pyran-6-carboxamide | | (morpholin-4-yl)propane-1,3-diol |
| 85A* | (R)-N-(5-(5-amino-1H-pyrazol-1- | | in Step 2 instead of tert-butyl(2- |
| | yl)-1,3,4-thiadiazol-2-yl)-4-(2,6- | | hydroxy-3- |
| | dimethoxyphenyl)-3-(3-hydroxy-2- | | methoxypropoxy)diphenylsilane. |
| | morpholinopropoxy)-2-oxo-2H- | | 2-(morpholin-4-yl)propane-1,3-diol |
| | pyran-6-carboxamide | | could be prepared starting from 3- |
| 85B* | (S)-N-(5-(5-amino-1H-pyrazol-1- | | (benzyloxy)propane-1,2-diol using |
| | yl)-1,3,4-thiadiazol-2-yl)-4-(2,6- | | a modified silyl protection |
| | dimethoxyphenyl)-3-(3-hydroxy-2- | | described in step 1 of Example 43 |
| | morpholinopropoxy)-2-oxo-2H- | | (Et₃N and DMAP replacing |
| | pyran-6-carboxamide | | imidazole), followed by |
| | | | mesylation, followed by SN₂ |
| | | | reaction using morpholine as the |
| | | | nucleophile and deprotection of the |
| | | | silyl group using TBAF. |
| 86A* | (R)-N-(5-(5-amino-1H-pyrazol-1- | — | Compounds 86A* and 86B* may |
| | yl)-1,3,4-thiadiazol-2-yl)-4-(2,6- | | be prepared according to Example |
| | dimethoxyphenyl)-3-(3-methoxy- | | 33 using 2-(benzyloxy)-3- |
| | 2-morpholinopropoxy)-2-oxo-2H- | | methoxypropan-1-ol (made |
| | pyran-6-carboxamide | | following reported conditions in J. |
| 86B* | (S)-N-(5-(5-amino-1H-pyrazol-1- | — | Med. Chem. 1987, 30, 792-797.) in |
| | yl)-1,3,4-thiadiazol-2-yl)-4-(2,6- | | place of 1,4-dioxan-2-ylmethanol |
| | dimethoxyphenyl)-3-(3-methoxy- | | in Step 1. Then, before engaging |
| | 2-morpholinopropoxy)-2-oxo-2H- | | the material in step 3, additional |
| | pyran-6-carboxamide | | manipulation are needed; benzyl |
| | | | deprotection using conc. HCl (see |
| | | | Example 33, step 5) followed by |
| | | | mesylate formation (see Example |
| | | | 26, step 2) and SN₂ reaction using |
| | | | morpholine and triethylamine. |
| | | | Steps 3-6 of Example 33 could then |
| | | | be followed. |
| Rac-87 | N-(5-(5-amino-1H-pyrazol-1-yl)- | 600.2 | Compound 87 (comprising a |
| | 1,3,4-thiadiazol-2-yl)-4-(2,6- | | mixture of 87A* and 87B*) was |
| | dimethoxyphenyl)-3-((1-hydroxy- | | prepared according to Example 33 |
| | 3-morpholinopropan-2-yl)oxy)-2- | | using 1-[(tert- |
| | oxo-2H-pyran-6-carboxamide | | butyldiphenylsilyl)oxy]-3- |
| 87A* | (S)-N-(5-(5-amino-1H-pyrazol-1- | | (morpholin-4-yl)propan-2-ol |
| | yl)-1,3,4-thiadiazol-2-yl)-4-(2,6- | | (prepared following step 1 of |
| | dimethoxyphenyl)-3-((1-hydroxy- | | Example 37 using 3-(morpholin-4- |
| | 3-morpholinopropan-2-yl)oxy)-2- | | yl)propane-1,2-diol in place of 2- |
| | oxo-2H-pyran-6-carboxamide | | bromo-3- |
| 87B* | (R)-N-(5-(5-amino-1H-pyrazol-1- | | (hydroxymethyl)benzonitrile) in |
| | yl)-1,3,4-thiadiazol-2-yl)-4-(2,6- | | place of 1,4-dioxan-2-ylmethanol. |
| | dimethoxyphenyl)-3-((1-hydroxy- | | The compound was purified by |
| | 3-morpholinopropan-2-yl)oxy)-2- | | reversed-phase chromatography |
| | oxo-2H-pyran-6-carboxamide | | using the following conditions: |
| | | | Column, C18 silica gel; mobile |
| | | | phase, MeCN in Water (0.1% |
| | | | TFA), 20% to 45% gradient in 15 |
| | | | min; detector, UV 254 nm. |
| 88 | N-(5-(5-amino-1H-pyrazol-1-yl)- | 600.2 | Compound 88 was prepared |
| | 1,3,4-thiadiazol-2-yl)-4-(2,6- | | according to Example 33 using 3- |
| | dimethoxyphenyl)-2-oxo-3-(3- | | ((tert- |
| | thiomorpholinopropoxy)-2H- | | butyldimethylsilyl)oxy)propan-1-ol |
| | pyran-6-carboxamide | | in place of 1,4-dioxan-2- |
| | | | ylmethanol in step 1. Then, before |
| | | | engaging the material in step 3, |
| | | | additional manipulation are needed; |
| | | | silyl deprotection using conc. HCl |
| | | | (see Example 33, step 5) followed |
| | | | by mesylate formation (see |
| | | | Example 26, step 2) and SN₂ |
| | | | reaction using thiomorpholine and |
| | | | K₂CO₃ in DMF. Steps 3-5 of |
| | | | Example 33 were then followed. |
| 89 | 3-(2-(1,4-thiazepan-4-yl)ethoxy)- | 600.1 | Compound 89 was prepared |
| | N-(5-(5-amino-1H-pyrazol-1-yl)- | | according to Example 33 using 2- |
| | 1,3,4-thiadiazol-2-yl)-4-(2,6- | | (t-butoxy)ethanol in place of 1,4- |
| | dimethoxyphenyl)-2-oxo-2H- | | dioxan-2-ylmethanol in Step 1. |
| | pyran-6-carboxamide | | Then, before engaging the material |
| | | | in step 3, additional manipulation |
| | | | were followed: t-Bu deprotection |
| | | | using HCl (see Example 13, step 6) |
| | | | followed by mesylate formation |
| | | | (see Example 26, step 2) and SN₂ |
| | | | reaction using 1,4-thiazepane and |
| | | | K₂CO₃ in MeCN at 80° C. Steps 3- |
| | | | 5 of Example 33 were then |
| | | | followed. |
| 90 | N-(5-(5-amino-1H-pyrazol-1-yl)- | 600.1 | Compound 90 was prepared |
| | 1,3,4-thiadiazol-2-yl)-4-(2,6- | | according to Example 33 using tert- |
| | dimethoxyphenyl)-2-oxo-3-(2-oxo- | | butyl 2-hydroxyacetate in place of |
| | 2-thiomorpholinoethoxy)-2H- | | 1,4-dioxan-2-ylmethanol in Step 1. |
| | pyran-6-carboxamide | | Then, before engaging the material |
| | | | in step 3, additional manipulation |
| | | | were needed; t-Bu deprotection |
| | | | using HCl (see Example 13, step 6) |
| | | | followed by amide coupling (see |
| | | | Example 4, step 3) using |
| | | | thiomorpholine in place of |
| | | | ammonium chloride. Steps 3-5 of |
| | | | Example 33 were then followed. |
| 91A* | (R)-N-(5-(5-amino-1H-pyrazol-1- | 616.1 | Compounds 91A* and 91B* were |
| | yl)-1,3,4-thiadiazol-2-yl)-4-(2,6- | | prepared according to Example 33 |
| | dimethoxyphenyl)-3-(2-hydroxy-3- | | using 2-(benzyloxy)-3- |
| | thiomorpholinopropoxy)-2-oxo- | | thiomorpholinopropan-1-ol in place |
| | 2H-pyran-6-carboxamide | | of 1,4-dioxan-2-ylmethanol in Step |
| 91B* | (S)-N-(5-(5-amino-1H-pyrazol- | | 1. 2-(benzyloxy)-3- |
| | 1yl)-1,3,4-thiadiazol-2-yl)-4-(2,6- | | thiomorpholinopropan-1-ol could |
| | dimethoxyphenyl)-3-(2-hydroxy-3- | | be prepared using a 4 steps |
| | thiomorpholinopropoxy)-2-oxo- | | sequence; silyl protection (see |
| | 2H-pyran-6-carboxamide | | Example 27, step 1), followed by |
| | | | mesylate formation (see Example |
| | | | 26, step 2), neat SN₂ reaction using |
| | | | thiomorpholine and silyl |
| | | | deprotection using TBAF. Then, |
| | | | steps 3-5 of Example 33 were |
| | | | followed. |
| 92A* | (R)-N-(5-(5-amino-1H-pyrazol-1- | 559.2 | Compounds 92A* and 92B* were |
| | yl)-1,3,4-thiadiazol-2-yl)-4-(2,6- | | prepared according to Example 33 |
| | dimethoxyphenyl)-3-(3-hydroxy-2- | | using 2- |
| | (methoxymethyl)propoxy)-2-oxo- | | ((benzyloxy)methyl)propane-1,3- |
| | 2H-pyran-6-carboxamide | | diol in place of 1,4-dioxan-2- |
| 92B* | (S)-N-(5-(5-amino-1H-pyrazol-1- | 559.2 | ylmethanol in Step 1. |
| | yl)-1,3,4-thiadiazol-2-yl)-4-(2,6- | | |
| | dimethoxyphenyl)-3-(3-hydroxy-2- | | |
| | (methoxymethyl)propoxy)-2-oxo- | | |
| | 2H-pyran-6-carboxamide | | |
| 93A* | 3-(2-((1S,4S)-2-thia-5- | 598.2 | Compounds 93A* and 93B* were |
| | azabicyclo[2.2.1]heptan-5- | | prepared according to Example 33 |
| | yl)ethoxy)-N-(5-(5-amino-1H- | | using 2-(t-butoxy)ethanol in place |
| | pyrazol-1-yl)-1,3,4-thiadiazol-2- | | of 1,4-dioxan-2-ylmethanol in Step |
| | yl)-4-(2,6-dimethoxyphenyl)-2- | | 1. Then, before engaging the |
| | oxo-2H-pyran-6-carboxamide | | material in step 3, additional |
| 93B* | 3-(2-((1R,4R)-2-thia-5- | 598.1 | manipulation were needed; t-Bu |
| | azabicyclo[2.2.1]heptan-5- | | deprotection using HCl (see |
| | yl)ethoxy)-N-(5-(5-amino-1H- | | Example 13, step 6) followed by |
| | pyrazol-1-yl)-1,3,4-thiadiazol-2- | | mesylate formation (see Example |
| | yl)-4-(2,6-dimethoxyphenyl)-2- | | 26, step 2) and SN₂ reaction using |
| | oxo-2H-pyran-6-carboxamide | | 2-thia-5-azabicyclo[2.2.1 ]heptane |
| | | | hydrochloride and K₂CO₃ in MeCN |
| | | | at 80° C. Steps 3-6 of Example 33 |
| | | | were then followed. |
| 94 | 3-(2-(3-thia-6- | 598.2 | Compound 94 was prepared |
| | azabicyclo[3.1.1]heptan-6- | | according to Example 33 using 2- |
| | yl)ethoxy)-N-(5-(5-amino-1H- | | (t-butoxy)ethanol in place of 1,4- |
| | pyrazol-1-yl)-1,3,4-thiadiazol-2- | | dioxan-2-ylmethanol in Step 1. |
| | yl)-4-(2,6-dimethoxyphenyl)-2- | | Then, before engaging the material |
| | oxo-2H-pyran-6-carboxamide | | in step 3, additional manipulation |
| | | | were needed: t-Bu deprotection |
| | | | using HCl (see Example 13, step 6) |
| | | | followed by mesylate formation |
| | | | (see Example 26, step 2) and SN₂ |
| | | | reaction using 3-thia-6- |
| | | | azabicyclo[3.1.1]heptane and |
| | | | K₂CO₃ in MeCN at 80° C. Steps 3- |
| | | | 5 of Example 33 were then |
| | | | followed. |
| 95 | 3-(2-(9-thia-3- | 612.2 | Compound 95 was prepared |
| | azabicyclo[3.3.1]nonan-3- | | according to Example 33 using 2- |
| | yl)ethoxy)-N-(5-(5-amino-1H- | | (t-butoxy)ethanol in place of 1,4- |
| | pyrazol-1-yl)-1,3,4-thiadiazol-2- | | dioxan-2-ylmethanol in Step 1. |
| | yl)-4-(2,6-dimethoxyphenyl)-2- | | Then, before engaging the material |
| | oxo-2H-pyran-6-carboxamide | | in step 3, additional manipulation |
| | | | were followed: t-Bu deprotection |
| | | | using HCl (see Example 13, step 6) |
| | | | followed by mesylate formation |
| | | | (see Example 26, step 2) and SN₂ |
| | | | reaction using 9-thia-3- |
| | | | azabicyclo[3.3.1]nonane and |
| | | | K₂CO₃ in MeCN at 80° C. Steps 3- |
| | | | 5 of Example 33 were then |
| | | | followed. |
| 96A* | (S)-N-(5-(5-amino-1H-pyrazol-1- | 616.2 | Compounds 96A* and 96B* were |
| | yl)-1,3,4-thiadiazol-2-yl)-4-(2,6- | | prepared according to Example 33 |
| | dimethoxyphenyl)-3-(2-(3- | | using 2-(t-butoxy)ethanol in place |
| | (hydroxymethyl)thiomorpholino)ethoxy)- | | of 1,4-dioxan-2-ylmethanol in Step |
| | 2-oxo-2H-pyran-6-carboxamide | | 1. Then, before engaging the |
| 96B* | (R)-N-(5-(5-amino-1H-pyrazol-1- | | material in step 3, additional |
| | yl)-1,3,4-thiadiazol-2-yl)-4-(2,6- | | manipulation were followed: t-Bu |
| | dimethoxyphenyl)-3-(2-(3- | | deprotection using HCl (see |
| | (hydroxymethyl)thiomorpholino)ethoxy)- | | Example 13, step 6) followed by |
| | 2-oxo-2H-pyran-6-carboxamide | | mesylate formation (see Example |
| | | | 26, step 2) and SN₂ reaction using |
| | | | 3-(((tert- |
| | | | butyldiphenylsilyl)oxy)methyl)thio- |
| | | | morpholine (previously prepared |
| | | | following a 3-steps sequence: |
| | | | DIBAL-H reduction of 4-(tert- |
| | | | butyl) 3-ethyl thiomorpholine-3,4- |
| | | | dicarboxylate, followed by TBDPS |
| | | | protection using imidazole and |
| | | | TBDPSCl, and Boc deprotection |
| | | | using HCl) and K₂CO₃ in MeCN at |
| | | | 80° C. Steps 3-5 of Example 33 |
| | | | were then followed. |
| 97A* | N-(5-(5-amino-1H-pyrazol-1-yl)- | — | Compounds 97A*, 97B*, 97C* and |
| | 1,3,4-thiadiazol-2-yl)-4-(2,6- | | 97D* may be prepared according to |
| | dimethoxyphenyl)-3-(((1R,2R)-1- | | Example 33 using 1- |
| | hydroxy-2- | | (hydroxymethyl)-2- |
| | methoxycyclopentyl)methoxy)-2- | | methoxycyclopentan-1-ol |
| | oxo-2H-pyran-6-carboxamide | | (previously made following a |
| 97B* | N-(5-(5-amino-1H-pyrazol-1-yl)- | — | dihydroxylation using OsO₄ on 1- |
| | 1,3,4-thiadiazol-2-yl)-4-(2,6- | | methoxy-2-methylenecyclopentane, |
| | dimethoxyphenyl)-3-(((1R,2S)-1- | | the later being prepared following |
| | hydroxy-2- | | reference J. Organomet. Chem. |
| | methoxycyclopentyl)methoxy)-2- | | 1987, 321, 279-290) in place of |
| | oxo-2H-pyran-6-carboxamide | | 1,4-dioxan-2-ylmethanol in Step 1. |
| 97C* | N-(5-(5-amino-1H-pyrazol-1-yl)- | — | |
| | 1,3,4-thiadiazol-2-yl)-4-(2,6- | | |
| | dimethoxyphenyl)-3-(((1S,2R)-1- | | |
| | hydroxy-2- | | |
| | methoxycyclopentyl)methoxy)-2- | | |
| | oxo-2H-pyran-6-carboxamide | | |
| 97D* | N-(5-(5-amino-1H-pyrazol-1-yl)- | — | |
| | 1,3,4-thiadiazol-2-yl)-4-(2,6- | | |
| | dimethoxyphenyl)-3-(((1S,2S)-1- | | |
| | hydroxy-2- | | |
| | methoxycyclopentyl)methoxy)-2- | | |
| | oxo-2H-pyran-6-carboxamide | | |
| 98A* | N-(5-(5-amino-1H-pyrazol-1-yl)- | — | Compounds 98A* and 98B* may |
| | 1,3,4-thiadiazol-2-yl)-3-(((1s,4s)- | | be prepared according to Example |
| | 1,4- | | 33 using 4-((tert- |
| | dihydroxycyclohexyl)methoxy)-4- | | butyldiphenylsilyl)oxy)-1- |
| | (2,6-dimethoxyphenyl)-2-oxo-2H- | | (hydroxymethyl)cyclohexan-1-ol |
| | pyran-6-carboxamide | | (previously prepared using a 4- |
| 98B* | N-(5-(5-amino-1H-pyrazol-1-yl)- | — | steps sequence: TBDPS protection |
| | 1,3,4-thiadiazol-2-yl)-3-(((1r,4r)- | | of 4-hydroxycyclohexan-1-one |
| | 1,4- | | using TBDPSCl and imidazole in |
| | dihydroxycyclohexyl)methoxy)-4- | | DCM, followed by wittig |
| | (2,6-dimethoxyphenyl)-2-oxo-2H- | | olefination using |
| | pyran-6-carboxamide | | triphenylphosphonium bromide |
| | | | with n-butyl lithium in THE, |
| | | | epoxyde formation using m- |
| | | | chloroperoxybenzoic acid and then |
| | | | epoxyde opening using calcium |
| | | | ammonium nitrate in acetonitrile |
| | | | and water) in place of 1,4-dioxan- |
| | | | 2-ylmethanol in Step 1. |
| 99A* | N-(5-(5-amino-1H-pyrazol-1-yl)- | 599.2 | Compounds 99A* and 99B* were |
| | 1,3,4-thiadiazol-2-yl)-4-(2,6- | | prepared according to Example 33 |
| | dimethoxyphenyl)-3-(((1s,4s)-4- | | using (4-((tert- |
| | hydroxy-1- | | butyldiphenylsilyl)oxy)-1- |
| | methoxycyclohexyl)methoxy)-2- | | methoxycyclohexyl)methanol |
| | oxo-2H-pyran-6-carboxamide | | (previously prepared using a 3- |
| 99B* | N-(5-(5-amino-1H-pyrazol-1-yl)- | | steps sequence: NaBH₄ reduction |
| | 1,3,4-thiadiazol-2-yl)-4-(2,6- | | in MeOH, followed by TBDPS |
| | dimethoxyphenyl)-3-(((1r,4r)-4- | | protection of 4- |
| | hydroxy-1- | | hydroxycyclohexan-1-one using |
| | methoxycyclohexyl)methoxy)-2- | | TBDPSCl and imidazole in DMF |
| | oxo-2H-pyran-6-carboxamide | | and then ester reduction using |
| | | | lithium aluminium hydride) in |
| | | | place of 1,4-dioxan-2-ylmethanol |
| | | | in Step 1. |
| 100A* | (S)-N-(5-(5-amino-1H-pyrazol-1- | 616.2 | Compounds 100A* and 100B* |
| | yl)-1,3,4-thiadiazol-2-yl)-4-(2,6- | | were prepared according to |
| | dimethoxyphenyl)-3-(2-(2- | | Example 33 using 2-(t- |
| | (hydroxymethyl)thiomorpholino)ethoxy)- | | butoxy)ethanol in place of 1,4- |
| | 2-oxo-2H-pyran-6-carboxamide | | dioxan-2-ylmethanol in Step 1. |
| 100B* | (R)-N-(5-(5-amino-1H-pyrazol-1- | | Then, before engaging the material |
| | yl)-1,3,4-thiadiazol-2-yl)-4-(2,6- | | in step 3, additional manipulation |
| | dimethoxyphenyl)-3-(2-(2- | | were followed: t-Bu deprotection |
| | (hydroxymethyl)thiomorpholino)ethoxy)- | | using HCl (see Example 13, step 6) |
| | 2-oxo-2H-pyran-6-carboxamide | | followed by mesylate formation |
| | | | (see Example 26, step 2) and SN₂ |
| | | | reaction using 2-(((tert- |
| | | | butyldiphenylsilyl)oxy)methyl)thio- |
| | | | morpholine (previously prepared |
| | | | following a 3-steps sequence: |
| | | | DIBAL-H reduction of 4-(tert- |
| | | | butyl) 2-ethyl thiomorpholine-2,4- |
| | | | dicarboxylate, followed by TBDPS |
| | | | protection using imidazole and |
| | | | TBDPSCl, and Boc deprotection |
| | | | using HCl) and K₂CO₃ in MeCN at |
| | | | 80° C. Steps 3-5 of Example 33 |
| | | | were then followed. |
| 101A | (R)-N-(5-(5-amino-1H-pyrazol-1- | — | Compound 101A may be prepared |
| | yl)-1,3,4-thiadiazol-2-yl)-4-(2,6- | | according to Example 33 using (R)- |
| | dimethoxyphenyl)-3-(oxetan-2- | | oxetan-2-ylmethanol in place of |
| | ylmethoxy)-2-oxo-2H-pyran-6- | | 1,4-dioxan-2-ylmethanol in Step 1. |
| | carboxamide | | |
| 101B | (S)-N-(5-(5-amino-1H-pyrazol-1- | — | Compound 101B may be prepared |
| | yl)-1,3,4-thiadiazol-2-yl)-4-(2,6- | | according to Example 33 using (S)- |
| | dimethoxyphenyl)-3-(oxctan-2- | | oxetan-2-ylmethanol in place of |
| | ylmethoxy)-2-oxo-2H-pyran-6- | | 1,4-dioxan-2-ylmethanol in Step 1. |
| | carboxamide | | |
| 102A* | N-(5-(5-amino-1H-pyrazol-1-yl)- | 571.2 | Compound 102A* was prepared |
| | 1,3,4-thiadiazol-2-yl)-4-(2,6- | | according to Example 33 using 2- |
| | dimethoxyphenyl)-3-(2-((1s,3s)-3- | | (t-butoxy)ethanol in place of 1,4- |
| | hydroxycyclobutoxy)ethoxy)-2- | | dioxan-2-ylmethanol in Step 1. |
| | oxo-2H-pyran-6-carboxamide | | Then, before engaging the material |
| | | | in step 3, additional manipulation |
| | | | were followed: t-Bu deprotection |
| | | | using HCl (see Example 13, step 6) |
| | | | followed by mesylate formation |
| | | | (see Example 26, step 2) and SN₂ |
| | | | reaction using cis-1,3- |
| | | | cyclobutanediol and sodium |
| | | | hydride in THF. Steps 3-5 of |
| | | | Example 33 were then followed. |
| 102B* | N-(5-(5-amino-1H-pyrazol-1-yl)- | 571.2 | Compound 102B* was prepared |
| | 1,3,4-thiadiazol-2-yl)-4-(2,6- | | according to Example 33 using 2- |
| | dimethoxyphenyl)-3-(2-((1r,3r)-3- | | (t-butoxy)ethanol in place of 1,4- |
| | hydroxycyclobutoxy)ethoxy)-2- | | dioxan-2-ylmethanol in Step 1. |
| | oxo-2H-pyran-6-carboxamide | | Then, before engaging the material |
| | | | in step 3, additional manipulation |
| | | | were needed; t-Bu deprotection |
| | | | using HCl (see Example 13, step 6) |
| | | | followed by mesylate formation |
| | | | (see Example 26, step 2) and SN₂ |
| | | | reaction using trans-1,3- |
| | | | cyclobutanediol and sodium |
| | | | hydride in THF. Steps 3-5 of |
| | | | Example 33 could then be |
| | | | followed. |
| 103 | N-(5-(5-amino-1H-pyrazol-1-yl)- | — | Compound 103 may be prepared |
| | 1,3,4-thiadiazol-2-yl)-4-(3- | | according to Example 47 using 2- |
| | methoxypyridin-2-yl)-2-oxo-3-(2- | | (t-butoxy)ethanol in place of 3- |
| | (thietan-3-yloxy)ethoxy)-2H- | | methoxy-2,2-dimethylpropan-1-ol |
| | pyran-6-carboxamide | | in Step 1. Then, before engaging |
| | | | the material in step 3, additional |
| | | | manipulation were needed; t-Bu |
| | | | deprotection using HCl (see |
| | | | Example 13, step 6) followed by |
| | | | mesylate formation (see Example |
| | | | 26, step 2) and SN₂ reaction using |
| | | | 3-thietanol and sodium hydride in |
| | | | THF. Steps 3-5 of Example 33 |
| | | | could then be followed. |
| 104 | N-(5-(5-amino-1H-pyrazol-1-yl)- | — | Compound 104 may be prepared |
| | 1,3,4-thiadiazol-2-yl)-4-(3- | | according to Example 47 using 2- |
| | methoxypyridin-2-yl)-2-oxo-3-(2- | | (t-butoxy)ethanol in place of 3- |
| | ((tetrahydro-2H-thiopyran-4- | | methoxy-2,2-dimethylpropan-1-ol |
| | yl)oxy)ethoxy)-2H-pyran-6- | | in Step 1. Then, before engaging |
| | carboxamide | | the material in step 3, additional |
| | | | manipulation were needed; t-Bu |
| | | | deprotection using HCl (see |
| | | | Example 13, step 6) followed by |
| | | | mesylate formation (see Example |
| | | | 26, step 2) and SN₂ reaction using |
| | | | tetrahydro-2H-thiopyran-4-ol and |
| | | | sodium hydride in THF. Steps 3-5 |
| | | | of Example 33 could then be |
| | | | followed. |
| 105A* | (R)-N-(5-(5-amino-1H-pyrazol-1- | — | Compounds 105A* and 105B* |
| | yl)-1,3,4-thiadiazol-2-yl)-4-(2,6- | | may be prepared according to |
| | dimethoxyphenyl)-3-(2-hydroxy-2- | | Example 33 using 2-((tert- |
| | (oxetan-3-yl)ethoxy)-2-oxo-2H- | | butyldimethylsilyl)oxy)-2-(oxetan- |
| | pyran-6-carboxamide | | 3-yl)ethan-1-ol (previously |
| 105B* | (S)-N-(5-(5-amino-1H-pyrazol-1- | — | prepared in a 2 steps sequence: |
| | yl)-1,3,4-thiadiazol-2-yl)-4-(2,6- | | silyl protection on ethyl 2-hydroxy- |
| | dimethoxyphenyl)-3-(2-hydroxy-2- | | 2-(oxetan-3-yl)acetate (see step 1 |
| | (oxetan-3-yl)ethoxy)-2-oxo-2H- | | of Example 37) followed by ester |
| | pyran-6-carboxamide | | reduction using lithium aluminium |
| | | | hydride) in place of 1,4-dioxan-2- |
| | | | ylmethanol in Step 1. Steps 3-6 of |
| | | | Example 33 could then be |
| | | | followed. |
| 106A* | (S)-N-(5-(5-amino-1H-pyrazol- | 541.2 | Compounds 106A* and 106B* |
| | 1yl)-1,3,4-thiadiazol-2-yl)-3-(1- | | were prepared according to |
| | cyclopropyl-2-hydroxyethoxy)-4- | | Example 33 using 2-((tert- |
| | (2,6-dimethoxyphenyl)-2-oxo-2H- | | butyldimethylsilyl)oxy)-1- |
| | pyran-6-carboxamide | | cyclopropylethan-1-ol, previously |
| 106B* | (R)-N-(5-(5-amino-1H-pyrazol-1- | 541.2 | prepared by doing a silyl protection |
| | yl)-1,3,4-thiadiazol-2-yl)-3-(1- | | on 1-cyclopropyl-1,2-ethanediol |
| | cyclopropyl-2-hydroxyethoxy)-4- | | (see step 1 of Example 37), in place |
| | (2,6-dimethoxyphenyl)-2-oxo-2H- | | of 1,4-dioxan-2-ylmethanol in Step |
| | pyran-6-carboxamide | | 1. Steps 2-6 of Example 33 were |
| | | | then followed. |
| 107B | (R)-N-(5-(5-amino-1H-pyrazol-1- | 486.1 | Compound 107B was prepared |
| | yl)-1,3,4-thiadiazol-2-yl)-3-((1- | | according to Example 49 using |
| | hydroxypropan-2-yl)oxy)-4-(3- | | (2S)-1-(benzyloxy)propan-2-ol in |
| | methoxypyridin-2-yl)-2-oxo-2H- | | place of (2R)-1- |
| | pyran-6-carboxamide | | (benzyloxy)propan-2-ol. |
| 108B | N-(5-(5-amino-1H-pyrazol-1-yl)- | 555.2 | Compound 108B was prepared |
| | 1,3,4-thiadiazol-2-yl)-4-(2,6- | | according to Example 50 using |
| | dimethoxyphenyl)-3-(((1s,4s)-4- | | (1r,4r)-cyclohexane-1,4-diol in |
| | hydroxycyclohexyl)oxy)-2-oxo- | | place of (1s,4s)-cyclohexane-1,4- |
| | 2H-pyran-6-carboxamide | | diol. |
| 109A* | N-(5-(5-amino-1H-pyrazol-1-yl)- | 555.1 | Compounds 109A* and 109B* |
| | 1,3,4-thiadiazol-2-yl)-4-(2,6- | | were prepared according to |
| | dimethoxyphenyl)-3-(((1R,3R)-3- | | Example 50 using (cis)- |
| | hydroxycyclohexyl)oxy)-2-oxo- | | cyclohexane-1,3-diol in place of |
| | 2H-pyran-6-carboxamide | | (1s,4s)-cyclohexane-1,4-diol. After |
| 109B* | N-(5-(5-amino-1H-pyrazol-1-yl)- | 555.1 | step 5, both enantiomers were |
| | 1,3,4-thiadiazol-2-yl)-4-(2,6- | | separated by prep-chiral-HPLC |
| | dimethoxyphenyl)-3-(((1S,3S)-3- | | with the following conditions |
| | hydroxycyclohexyl)oxy)-2-oxo- | | (column: CHIRALPAK SA 2*25 |
| | 2H-pyran-6-carboxamide | | cm, 5 μm; mobile phase A: |
| | | | Hexanes (0.1% TFA), mobile |
| | | | phase B: (ethanol:DCM = 1:1); |
| | | | flow rate: 20 mL/min; gradient: |
| | | | isocratic 20%. The first eluting |
| | | | peak RT(min): 12.234 was used to |
| | | | make 109A* and the second eluting |
| | | | peak (RT2(min): 16.927) was used |
| | | | to make 109B* following step 6 of |
| | | | Example 50. |
| 110A* | N-(5-(5-amino-1H-pyrazol-1-yl)- | 555.1 | Compounds 110A* and 110B* |
| | 1,3,4-thiadiazol-2-yl)-4-(2,6- | | were prepared according to |
| | dimethoxyphenyl)-3-(((1R,2R)-2- | | Example 50 using (cis)- |
| | hydroxycyclohexyl)oxy)-2-oxo- | | cyclohexane-1,2-diol in place of |
| | 2H-pyran-6-carboxamide | | (1s,4s)-cyclohexane-1,4-diol. After |
| 110B* | N-(5-(5-amino-1H-pyrazol-1-yl)- | 555.1 | step 5, both enantiomers were |
| | 1,3,4-thiadiazol-2-yl)-4-(2,6- | | separated by prep-chiral-HPLC |
| | dimethoxyphenyl)-3-(((1S,2S)-2- | | with the following conditions |
| | hydroxycyclohexyl)oxy)-2-oxo- | | (column: CHIRALPAK SA 2*25 |
| | 2H-pyran-6-carboxamide | | cm, 5 μm; mobile phase A: |
| | | | Hexanes (0.2% TFA), mobile |
| | | | phase B: (ethanol:DCM = 1:1); |
| | | | flow rate: 20 mL/min; gradient: |
| | | | isocratic 25%. The first eluting |
| | | | peak (RT(min): 8.84) was used to |
| | | | make 110A* and the second eluting |
| | | | peak (RT(min): 11.41) was used to |
| | | | make 110B* following step 6 of |
| | | | Example 50. |
| 112A* | N-(5-(5-amino-1H-pyrazol-1-yl)- | 512.2 | Compounds 112A* and 112B* |
| | 1,3,4-thiadiazol-2-yl)-3-(((1R,3R)- | | were prepared according to |
| | 3-hydroxycyclopentyl)oxy)-4-(3- | | Example 51 using (cis)- |
| | methoxypyridin-2-yl)-2-oxo-2H- | | cyclohexane-1,3-diol in place of |
| | pyran-6-carboxamide | | trans-cyclopentane-1,3-diol. After |
| 112B* | N-(5-(5-amino-1H-pyrazol-1-yl)- | 512.2 | step 5, both enantiomers were |
| | 1,3,4-thiadiazol-2-yl)-3-(((1S,3S)- | | separated by prep-chiral-HPLC |
| | 3-hydroxycyclopentyl)oxy)-4-(3- | | with the following conditions |
| | methoxypyridin-2-yl)-2-oxo-2H- | | (column: CHIRALPAK SA 2*25 |
| | pyran-6-carboxamide | | cm, 5 μm; mobile phase A: |
| | | | Hexanes (0.2% TFA), mobile |
| | | | phase B: (ethanol:DCM = 1:1); |
| | | | flow rate: 20 mL/min; gradient: |
| | | | isocratic 60%. The first eluting |
| | | | peak (RT(min): 9.20) was used to |
| | | | make 112A* and the second eluting |
| | | | peak (RT(min): 12.03) was used to |
| | | | make 112B* |

### Assay Methods

cGAS catalyzes the cyclization of ATP and GTP to produce cGAMP, which is the activating ligand of STING. Human cGAS (hcGAS) inhibition can thus be quantified by measuring how much cGAMP is formed either indirectly, by monitoring ATP-depletion as in the hcGAS Kinase-Glo assay (as described below), or directly, such as using the hcGAS LCMS assay (as described below).

1. hcGAS Kinase-Glo Assay

Compounds may be tested for their human-cGAS (h-cGAS) inhibition activity using the methodology reported in Lama et al., “Development of human cGAS-specific small molecule inhibitors for repression of dsDNA-triggered interferon expression”, Nature Communications 10, Article number: 2261 (2019), with slight changes to some conditions as shown in Table B.

TABLE B
Summary of hcGAS Kinase-Glo assay conditions

| Reagent/Conditions | | Lama et al. | Changes to Lama |
|---|---|---|---|
| Enzyme | hcGAS (nM) | 100 | 40 |
| Buffer | Tris(hydroxymethyl)aminomethane | 20 | 20 |
| | hydrochloride (Tris-HCl) pH 7.4 (mM) | | |
| | MgCl₂ (mM) | 5 | 10 |
| | NaCl (mM) | 150 | 25 |
| | Tween ™-20 (%) | 0.01 | 0.01 |
| | ZnCl₂ (μM) | 1 | 1 |
| | Dithiothreitol (DTT) (mM) | 1 | 1 |
| | Dimethylsulfoxide (DMSO) (%) | 0.5 | 5 |
| Substrates | Adenosine triphosphate (ATP) (μM) | 100 | 100 |
| | Guanosine triphosphate (GTP) (μM) | 100 | 100 |
| | Double stranded DNA (dsDNA) (nM) | 25 | 25 |
| Assay | Plate (wells) | 384 | 384 |
| | Incubation length (h) | 7 | 3 |
| | Total volume (μL) | 20 | 20 |
| | Kinase-Glo Max (μL) | 20 | 20 |

2. hGAS LCMS Assay Conditions

Compounds were tested for their hcGAS inhibition activity using direct measurement of cGAMP production by LC/MS. Briefly, compounds were incubated with enzyme and substrates for 4 hours (see Table C), before the reaction was stopped with 3 volumes of 70/30 Acetonitrile/H₂O mix containing 0.15 uM cGAMP-¹³C₁₀¹⁵N₅ as internal standard and mixed for 5 mmn. After centrifugation (3700 rpm, 10 mM, 100 Celsius), 100 uL of each reaction was collected and mixed to equal volume of acetonitrile (MeCN). Samples were then analyzed on Qexactive (Column: XBridge BEH Amide Column, 130 Å, 3.5 μm, 3 mm×50 mm. Mobile phase A: 10 mM Ammonium acetate in 95/5 H₂O/MeCN (0.5% DMSO), Mobile phase B: MeCN (0.5% DMSO).

The results of the assay, expressed as IC₅₀ values, are reported in Table D with the following designations: A represents an IC₅₀ value <0.005 μM (with A1 representing an IC₅₀ value <0.0005 μM, A2 representing an IC₅₀ value ≥0.0005 μM and <0.001 μM, and A3 representing an IC₅₀ value ≥0.001 μM and <0.005 μM); B represents an IC₅₀ value ≥0.005 μM and <0.02 μM; C represents an IC₅₀ value ≥0.02 μM and <0.06 μM; and D represents an IC₅₀ value ≥0.06 μM.

TABLE C
Summary of hcGAS LCMS assay conditions

| | | |
|---|---|---|
| Enzyme | hcGAS (nM) | 0.5 |
| Buffer | Tris(hydroxymethyl)aminomethane | 20 |
| | hydrochloride (Tris-HCl) pH 7.4 (mM) | |
| | MgCl₂ (mM) | 10 |
| | NaCl (mM) | 25 |
| | Tween ™-20 (%) | 0.01 |
| | ZnCl₂ (μM) | 1 |
| | Dithiothreitol (DTT) (mM) | 1 |
| | Dimethylsulfoxide (DMSO) (%) | 5 |
| Substrates | ATP (μM) | 100 |
| | Guanosine triphosphate (GTP) (μM) | 100 |
| | Double stranded DNA (dsDNA) (nM) | 25 |
| Assay | Plate (wells) | 96 |
| | Incubation length (h) | 4 |
| | Total volume (μL) | 40 |

### 3. Activity Data

Activity data obtained from the above described hcGAS LCMS assay method is provided in

TABLE D
Dashed lines (—) indicate no data available.
Table D. Activity Data

| | hcGAS LCMS |
|---|---|
| Comp'd # | IC₅₀ (uM) |
| 1 | A2 |
| 2 | B |
| 3 | C |
| 4 | B |
| 5 | A1 |
| 6 | A2 |
| 6-Ac | — |
| 7 | A3 |
| 8 | A2 |
| 8-Ac | — |
| 9 | A2 |
| 9-Ac | — |
| 10 | A3 |
| 10-Ac | — |
| 11 | A2 |
| 12 | A2 |
| 12-Ac | — |
| 13 | A2 |
| 14 | C |
| 15A* | A1 |
| 15B* | A1 |
| 15-Ac | — |
| 16 | — |
| 17 | A1 |
| 17-Ac-OtBu | — |
| 18 | A3 |
| 19 | A3 |
| 19-Ac | — |
| 20 | A3 |
| 20-Ac | — |
| 21 | A3 |
| 21-Ac | — |
| 22 | A3 |
| 22-Ac | — |
| 23 | A2 |
| 24A* | A1 |
| 24B* | A1 |
| 24-Ac | — |
| 25 | — |
| 25-Ac | — |
| 26 | A1 |
| 26-OtBu | — |
| 27 | A3 |
| 27-Ac | — |
| 28 | A3 |
| 28-Ac | — |
| 29 | A3 |
| 29-Ac | — |
| 30 | A2 |
| 31 | B |
| 32 | A3 |
| 32-Ac | — |
| 33 | A3 |
| 34A* | A1 |
| 34B* | A1 |
| 34-Ac-OtBu | — |
| 35 | — |
| 35-Ac | — |
| 36 | A2 |
| 37A* | A2 |
| 37B* | A3 |
| 37-Ac | — |
| 38A* | A2 |
| 38B* | A2 |
| 39 | A3 |
| 39-Ac | — |
| 40 | A2 |
| 40-Ac | — |
| 41 | A1 |
| 42 | A2 |
| 43A* | A2 |
| 43B | A2 |
| 43-Ac | — |
| 44 | A1 |
| 45 | A1 |
| 45-Ac | — |
| 46A | A2 |
| 46B | A1 |
| 47A | A3 |
| 47A-Ac | — |
| 47B | A1 |
| 47B-Ac | — |
| 48 | A2 |
| 49 | A3 |
| 50 | A1 |
| 51 | A2 |
| 51-Ac | — |
| 52A* | A1 |
| 52B* | A2 |
| 53 | A1 |
| 54 | A2 |
| 55A* | A2 |
| 55B* | A2 |
| 55-Ac-OtBu | — |
| 56A* | A2 |
| 56B* | A2 |
| 57A | A2 |
| 57B | A1 |
| 58 | A2 |
| 59 | A1 |
| 59-Ac | — |
| 60 | A3 |
| 60-Ac | — |
| 61 | A3 |
| 62A* | A2 |
| 62B* | A2 |
| 63A* | A2 |
| 63B* | A2 |
| 64A*/B* | A2 |
| 64C*/D* | A2 |
| 65 | A2 |
| 66 | A2 |
| 67 | A3 |
| 67-Ac | — |
| 68 | A1 |
| 69 | B |
| 70 | A2 |
| 71A* | A2 |
| 71B* | A2 |
| 71-Ac | — |
| 72 | A2 |
| 73 | A3 |
| 73-Ac | — |
| 74 | A2 |
| 74-Ac | — |
| 75 | A2 |
| 76A* | — |
| 76B* | — |
| 77A* | — |
| 77B* | — |
| 78A*/78B* | B |
| 79A* | A3 |
| 79B* | A3 |
| 80A* | — |
| 80B* | — |
| 81A* | A3 |
| 81B* | A3 |
| 82A* | A3 |
| 82B* | A2 |
| 83A*/83B* | A2 |
| 84A*/84B* | B |
| Rac-85 | A3 |
| 86A* | — |
| 86B* | — |
| Rac-87 | A3 |
| 88 | A2 |
| 89 | A2 |
| 90 | A1 |
| 91A*/91B* | A2 |
| 92A* | A2 |
| 92B* | A3 |
| 93A* | A2 |
| 93B* | A2 |
| 94 | A2 |
| 95 | — |
| 96A*/96B* | A3 |
| 97A* | — |
| 97B* | — |
| 97C* | — |
| 97D* | — |
| 98A* | — |
| 98B* | — |
| 99A*/99B* | A3 |
| 100A*/100B* | A3 |
| 101A* | — |
| 101B* | — |
| 102A* | A2 |
| 102B* | A2 |
| 103 | — |
| 104 | — |
| 105A* | — |
| 105B* | — |
| 106A* | A3 |
| 106B* | A3 |
| 107A | A3 |
| 107A-Ac | — |
| 107B | A2 |
| 107B-Ac | — |
| 108A | A1 |
| 108B | A3 |
| 109A* | A3 |
| 109B* | A2 |
| 110A* | A3 |
| 110B* | A2 |
| 111A* | A2 |
| 111B* | A2 |
| 112A* | A3 |
| 112B* | A3 |
| 113-Ac | — |
| 113A* | A3 |
| 113B* | A2 |
| 113C* | D |
| 113D* | B |

### OTHER EMBODIMENTS

Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims or embodiments is introduced into another claim or embodiment. For example, any claim that is dependent on another claim can be modified to include one or more limitations found in any other claim that is dependent on the same base claim. Where the present disclosure recites elements presented as lists, e.g., in Markush group format, each subgroup of the elements is also disclosed, and any element(s) can be removed from the group. It should be further understood that when any variable (e.g., an R group) is present more than one time in a given Markush structure (e.g., 2 or more times), and that variable may be selected from a given list of two or more elements, unless otherwise stated or understood within the context of the present disclosure, that variable (e.g., the R group) at each repeated occurrence (e.g., 2 or more times) is independent of each other, being independently selected from that given list.

It should also be understood that, in general, where the present disclosure, or aspects of the present disclosure, is/are referred to as comprising particular elements and/or features, certain embodiments of the present disclosure or aspects of the present disclosure also consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are understood to be included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the present disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

The scope of the present embodiments described herein is not intended to be limited to the above Description, but also includes that as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 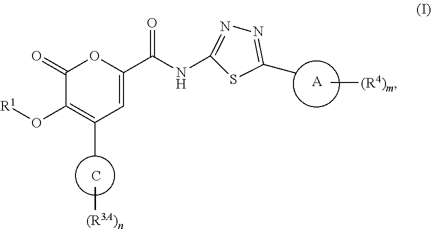
or a pharmaceutically acceptable salt thereof, wherein:
Ring A is a 5-membered monocyclic heteroaryl;
R¹ is C₁-C₆ alkyl, C₂-C₆ alkenyl, C₂-C₆ alkynyl, -L₃-(C₃-C₆ carbocyclyl), or -L₃-(4- to 10-membered heterocyclyl), wherein the alkyl, alkenyl, alkynyl, carbocyclyl, and heterocyclyl are independently substituted with 0, 1, 2, 3, or 4 R^{1A}; each R^{1A} is independently halogen, —OR^{1B}, —N(R^{1B})₂, —SR^{1B}, —C(═O)OR^{1B}, —C(═O)N(R^{1C})₂, —(C₁-C₃ alkylene)-OR^{1B}, or —(C₁-C₃ alkylene)-SR^{1B}, or two instances of R^{1A} are taken together to form ═O; each R^{1B} is independently hydrogen, C₁-C₄ alkyl, or C₁-C₄ haloalkyl, wherein the alkyl and haloalkyl are independently substituted with 0, 1, 2, 3, or 4 R^{1D}; each R^{1C} is independently hydrogen, C₁-C₃ alkyl, C₁-C₃ haloalkyl, or -ORiF; each R^{1D} is independently halogen, —OR^{1F}, or —N(R^{1F})₂; and each R^{1F} is independently hydrogen, C₁-C₃ alkyl, or C₁-C₃haloalkyl;
L₃ is a bond, C₁-C₃ alkylene, or —(C₁-C₃ alkylene)-O—, wherein the alkylene is independently substituted with 0, 1, 2, 3, or 4 R^{1E}; and each R^{1E} is independently —(C₁-C₃ alkylene)-OR^{1B} or —OR^{1B}, or two instances of R^{1E} are taken together to form ═O;
Ring C is a C₆-C₁₀ aryl or 5- to 10-membered heteroaryl;
each R^{3A} is independently C₁-C₃ alkyl, C₂-C₃ alkenyl, C₂-C₃ alkynyl, halogen, -L₁-CN, -L₁-SOR^{3C}, -L₁-SO₂R^{3C}, -L₁-SR^{3B}, -L₁-PO(R^{3C})₂, -L₁-OR^{3B}, -L₁-N(R^{3B})₂, -L₁-C(═O)N(R^{3B})₂, -L₁-C(═O)OR^{3B}, -L₁-(C₃-C₆ carbocyclyl), -L₁-(4- to 6-membered heterocyclyl), -L₁-(C₆₋₁₀ aryl), or -L₁-(5- to 10-membered heteroaryl), or two R^{3A} groups are joined, with the atoms to which they are attached, to form C₆ aryl, 5- to 6-membered heteroaryl, C₃-C₆ carbocyclyl, or 4- to 6-membered heterocyclyl, and wherein the alkyl, alkenyl, alkynyl, carbocyclyl, heterocyclyl, aryl, and heteroaryl are independently substituted with 0, 1, 2, 3, or 4 R^{3D};
each R^{3B} is independently hydrogen, C₁-C₃ alkyl, C₃-C₆ carbocyclyl, or 4- to 6-membered heterocyclyl, wherein the alkyl, carbocyclyl, and heterocyclyl are independently substituted with 0, 1, 2, 3, or 4 R^{3D};
each R^{3C} is independently C₁-C₃ alkyl or C₁-C₃ haloalkyl;
each R^{3D} is independently halogen, —OR^{3E}, —CN, C₁-C₃ alkyl, or C₁-C₃ haloalkyl;
each R^{3E} is independently hydrogen, C₁-C₄ alkyl, or C₁-C₄haloalkyl;
each R⁴ is independently halogen, —CN, -L₂-OR^{4A}, -L₂-N(R^{4B})₂, C₁-C₆ alkyl, or C₁-C₆ haloalkyl;
each R^{4A} and R^{4B} are independently hydrogen, C₁-C₃ alkyl, C₁-C₃ haloalkyl, or —C(═O)R^{4C}, wherein R^{4C} is C₁-C₆ alkyl or C₁-C₆ haloalkyl;
each L₁ and L₂ is independently a bond, C₁-C₃ alkylene, or C₁-C₃ haloalkylene;
m is 0, 1, or 2; and
n is 0, 1, 2, 3, or 4.

2. The compound of claim 1, wherein the compound is of Formula (I′): 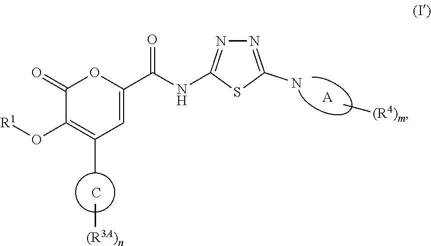
or a pharmaceutically acceptable salt thereof, wherein a nitrogen atom of the heteroaryl Ring A is directly linked to the thiadiazole moiety.

3. The compound of claim 1 or 2, or a pharmaceutically acceptable salt thereof, wherein Ring C is phenyl substituted with 0, 1, 2, 3, or 4 R^{3A}.

4. The compound of claim 1 or 2, or a pharmaceutically acceptable salt thereof, wherein Ring C is a 6-membered heteroaryl, wherein the heteroaryl has 1 ring N atom.

5. The compound of claim 1 or 2, or a pharmaceutically acceptable salt thereof, wherein Ring C is a 5-membered heteroaryl, wherein the heteroaryl has 2 ring N atoms.

6. The compound of any one of the preceding claims, or a pharmaceutically acceptable salt thereof, wherein Ring C is selected from one of the following formulae:

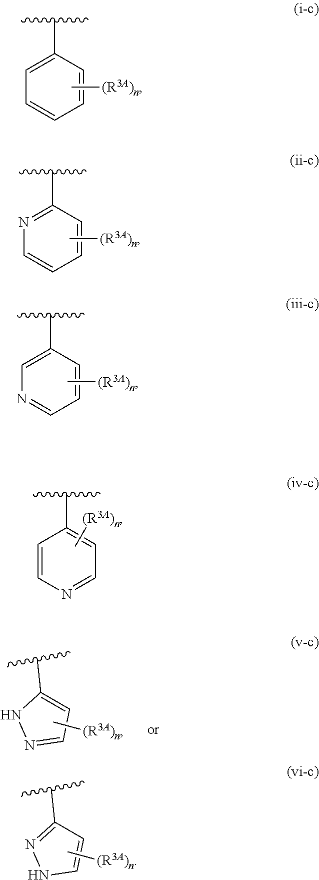

7. The compound of any one of the preceding claims, wherein the compound is of Formula (I-d): 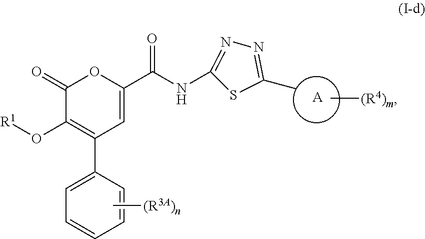
or a pharmaceutically acceptable salt thereof.

8. The compound of any one of the preceding claims, wherein the compound is of Formula (I-e-1), (I-e-2), or (I-e-3): 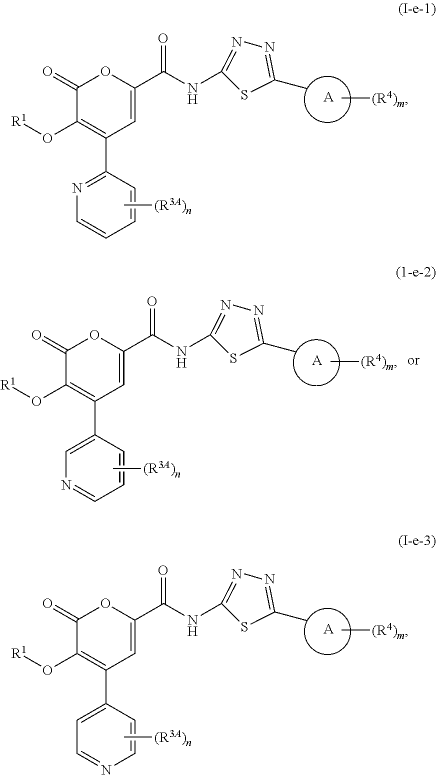
or a pharmaceutically acceptable salt thereof.

9. The compound of any one of the preceding claims, wherein the compound is of Formula (I-f-1) or (I-f-2): 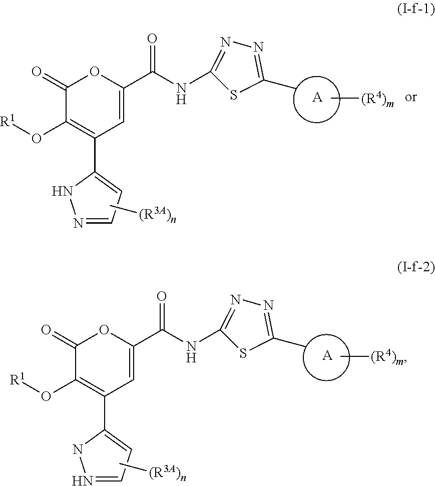
or a pharmaceutically acceptable salt thereof.

10. The compound of any one of the preceding claims, or a pharmaceutically acceptable salt thereof, wherein at least one R^{3A} is independently —CN, —OCH₃, —OCHF₂, —CH₃, or Cl.

11. The compound of any one of the preceding claims, or a pharmaceutically acceptable salt thereof, wherein Ring C is of one of the following formulae:

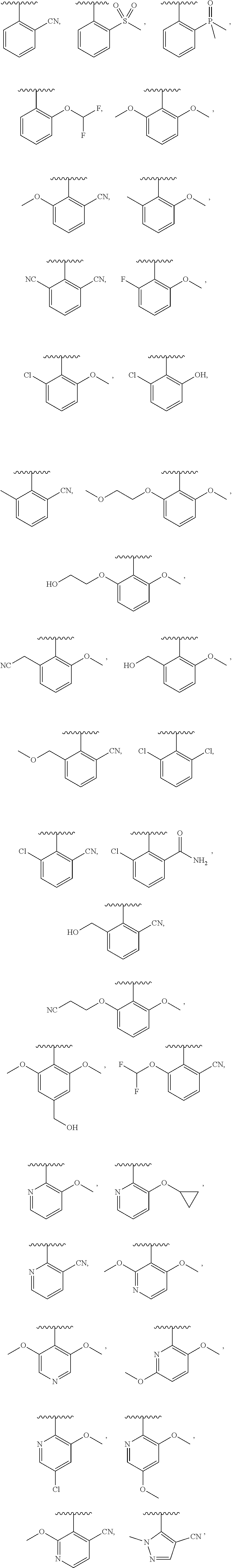

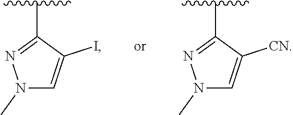

12. The compound of any one of the preceding claims, or a pharmaceutically acceptable salt thereof, wherein R¹ is C₁-C₆ alkyl substituted with 1 or 2 R^{1A}; each R^{1A} is independently —OR^{1B}; and each R^{1B} is independently hydrogen or C₁-C₃ alkyl substituted with 0 R^{1D}.

13. The compound of any one of the preceding claims, or a pharmaceutically acceptable salt thereof, wherein R¹ is -L₃-(C₃-C₆ carbocyclyl), wherein the carbocyclyl is substituted with 1 R^{1A}; R^{1A} is —OR^{1B}; R^{1B} is hydrogen; and L₃ is bond or —(C₁-C₃ alkylene)-O—.

14. The compound of any one of the preceding claims, or a pharmaceutically acceptable salt thereof, wherein R¹ is -L₃-(4- to 10-membered heterocyclyl), wherein the heterocyclyl is substituted with 0 R^{1A}; and L₃ is C₁-C₃ alkylene substituted with 0 R^{1E}.

15. The compound of any one of the preceding claims, or a pharmaceutically acceptable salt thereof, wherein R¹ is one of the following formulae:

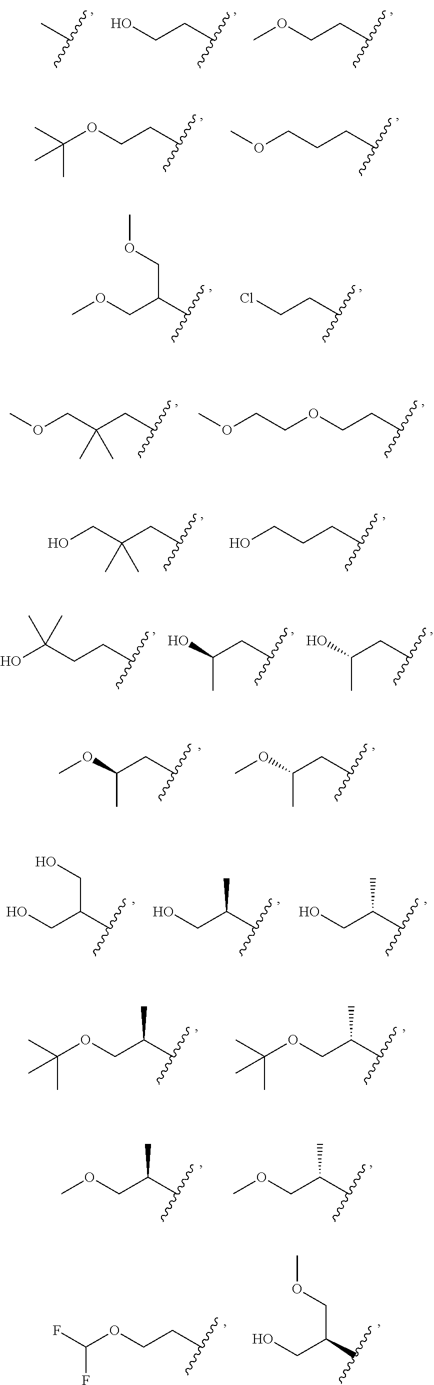

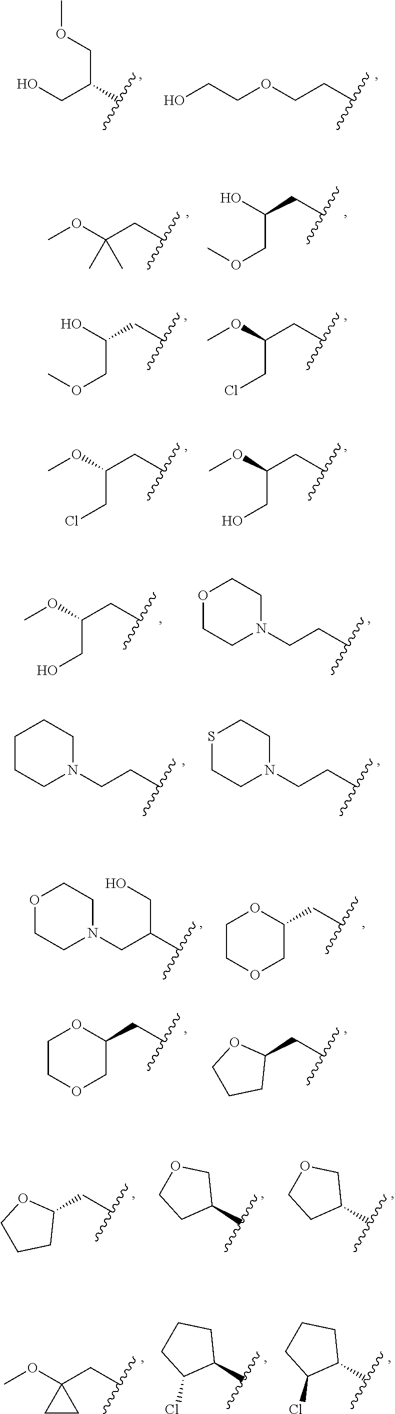

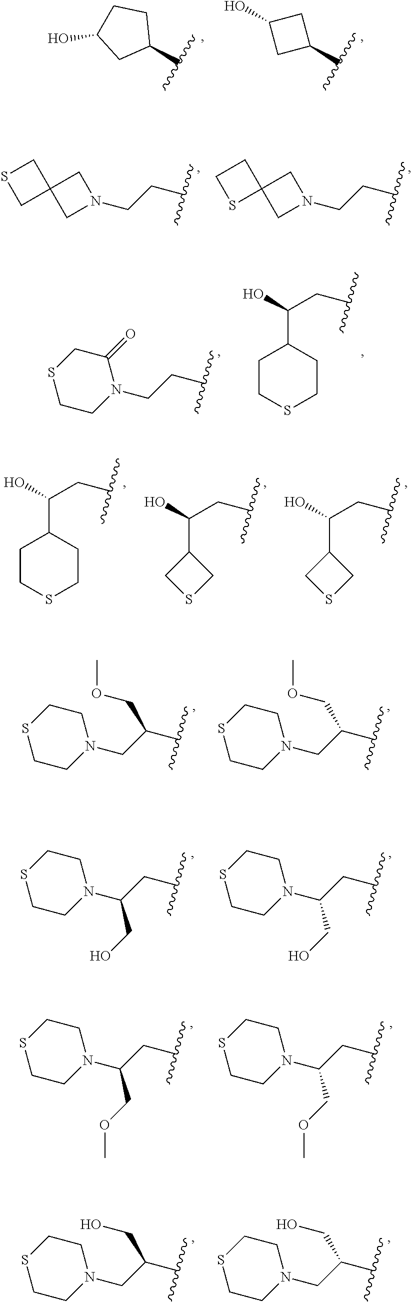

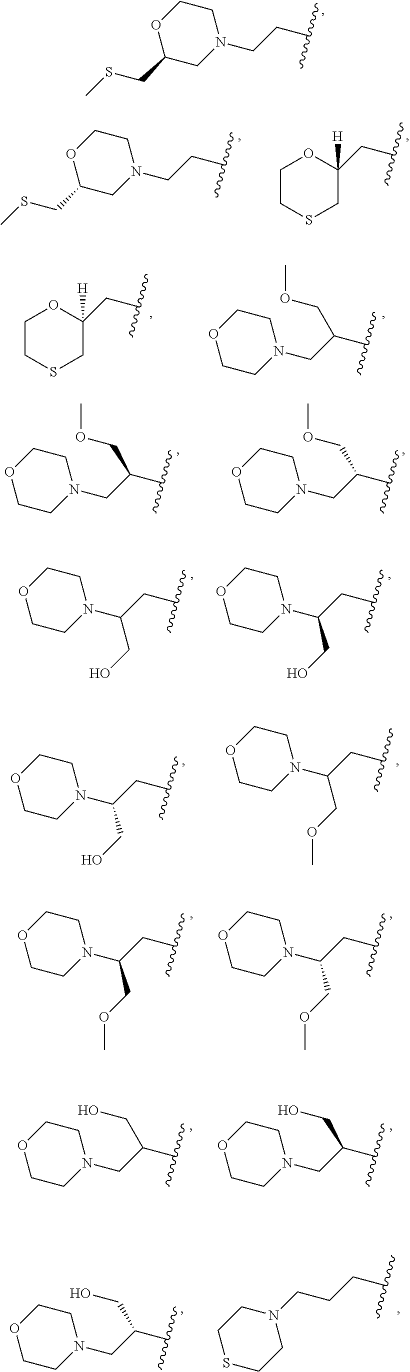

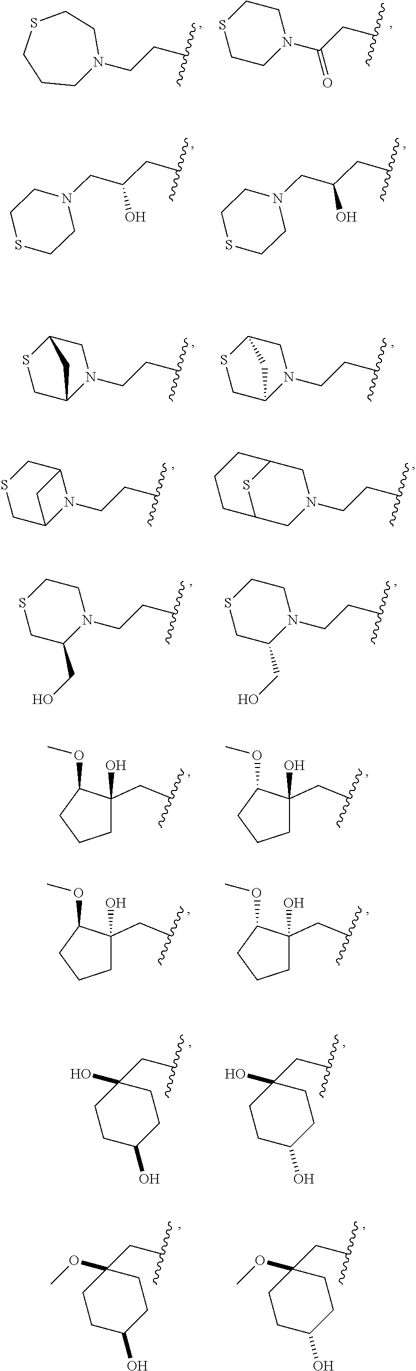

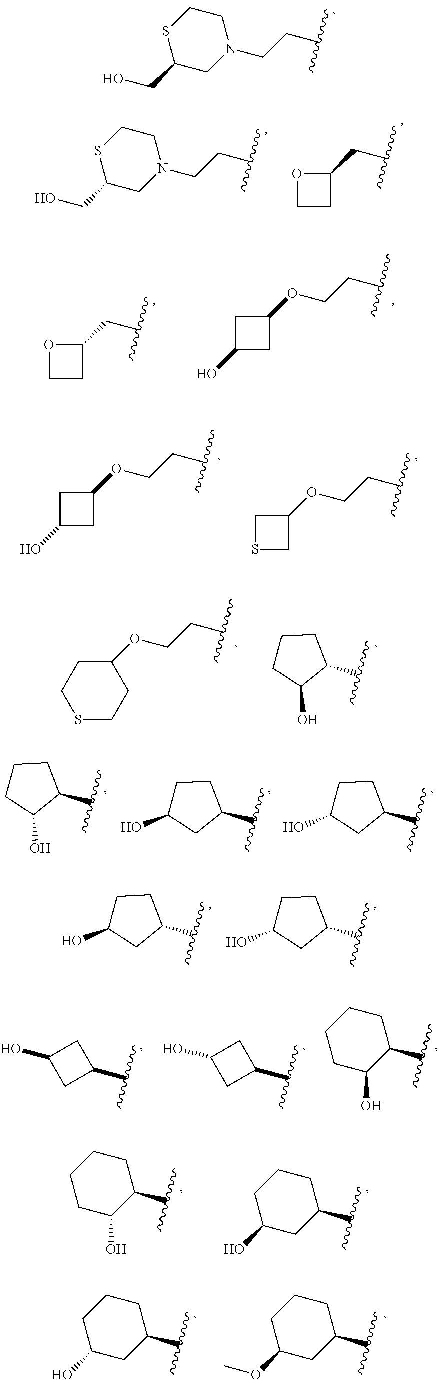

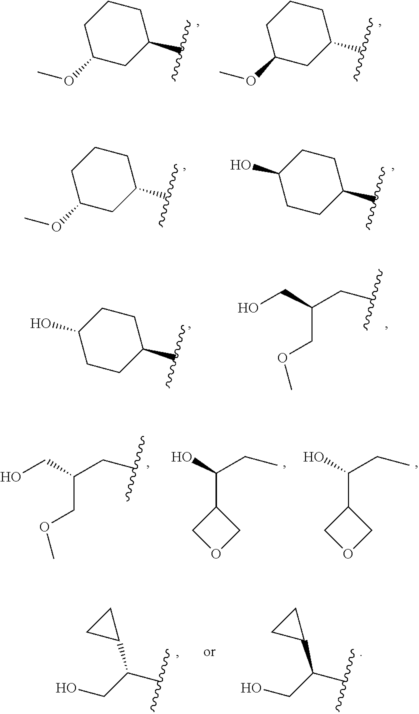

16. The compound of any one of the preceding claims, or a pharmaceutically acceptable salt thereof, wherein Ring A is one of the following formulae:

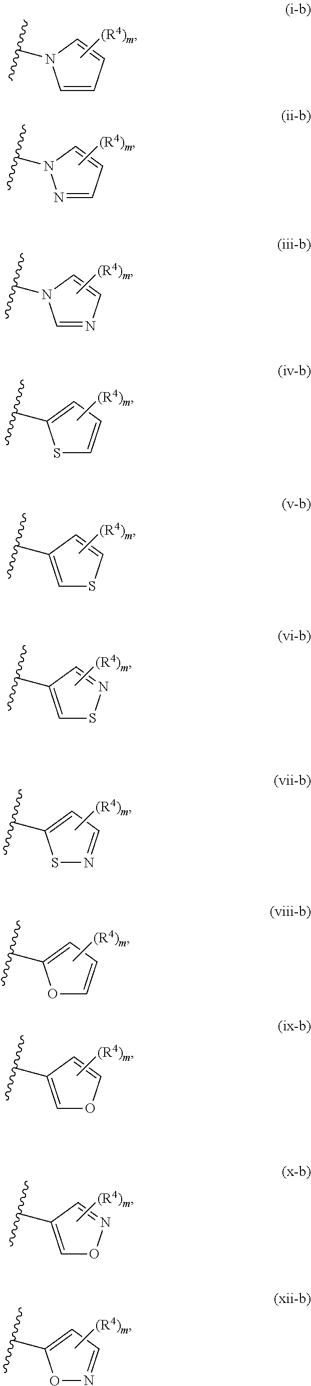

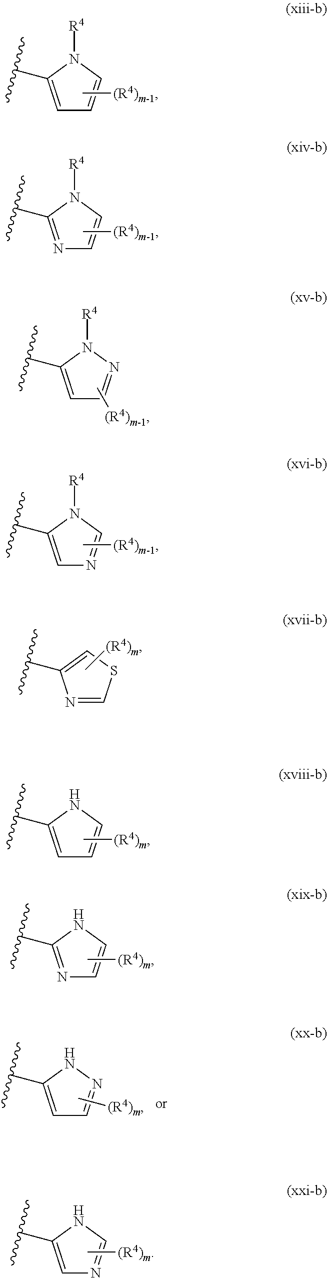

17. The compound of any one of the preceding claims, or a pharmaceutically acceptable salt thereof, wherein Ring A is one of the following formulae:

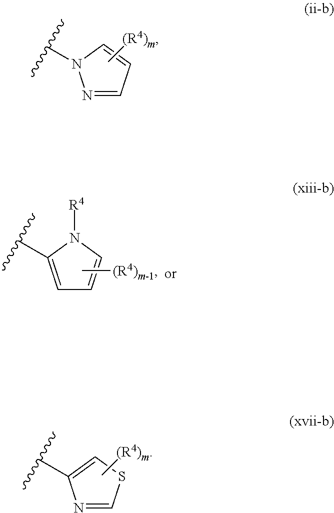

18. The compound of any one of the preceding claims, or a pharmaceutically acceptable salt thereof, wherein each R⁴ is independently halogen, —CN, -L₂-OR^{4A}, -L₂-N(R⁴)₂, C₁-C₆ alkyl, or C₁-C₆ haloalkyl.

19. The compound of any one of the preceding claims, or a pharmaceutically acceptable salt thereof, wherein each R⁴ is independently —CH₃ or —NH₂.

20. The compound of any one of the preceding claims, or a pharmaceutically acceptable salt thereof, wherein Ring A is:

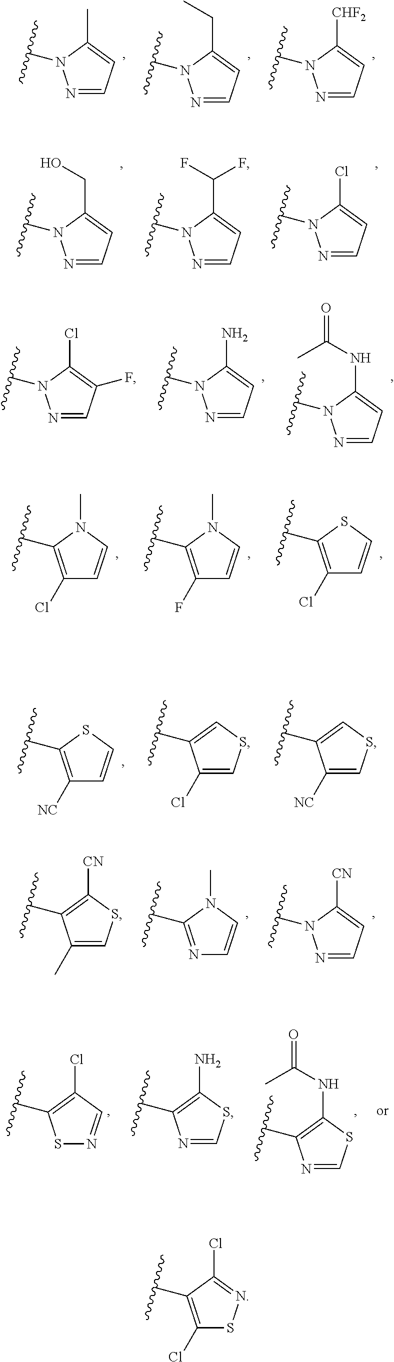

21. The compound of any one of the preceding claims, or a pharmaceutically acceptable salt thereof, wherein Ring A is:

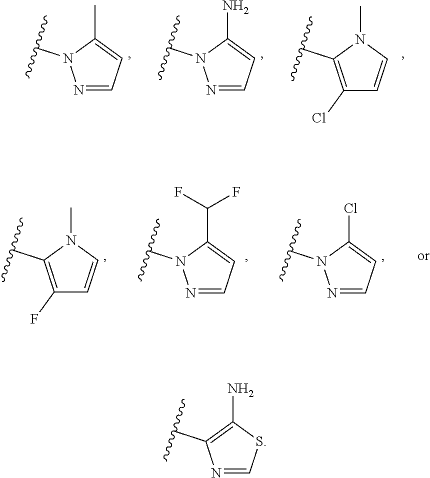

22. The compound of any one of the preceding claims, or a pharmaceutically acceptable salt thereof, wherein m is 1.

23. The compound of any one of the preceding claims, or a pharmaceutically acceptable salt thereof, wherein m is 2.

24. The compound of any one of the preceding claims, or a pharmaceutically acceptable salt thereof, wherein n is 1.

25. The compound of any one of the preceding claims, or a pharmaceutically acceptable salt thereof, wherein n is 2.

26. The compound of claim 1, wherein the compound is selected from those in Table 1 or Table 2, and pharmaceutically acceptable salts thereof.

27. A pharmaceutical composition comprising the compound of any one of the preceding claims, or a pharmaceutically acceptable salt thereof, and a pharmaceutically acceptable carrier.

28. A method of treating a disease or disorder in a subject in need thereof comprising administering to the subject a compound of any one of the preceding claims, or a pharmaceutically acceptable salt thereof, or a pharmaceutical composition thereof.

29. A method of modulating cGAS activity in a cell comprising contacting the cell with a compound of any one of the preceding claims, or a pharmaceutically acceptable salt thereof.

30. A method of preparing a compound of Formula (I): 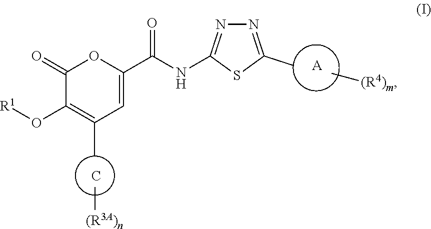
or a salt thereof, wherein Ring A, Ring C, R¹, R^{3A}, R⁴, m, and n are defined in claim 1, the method comprising peptide coupling of a compound of Formula (H-1), or salt thereof, with a compound of Formula (N), or salt thereof: 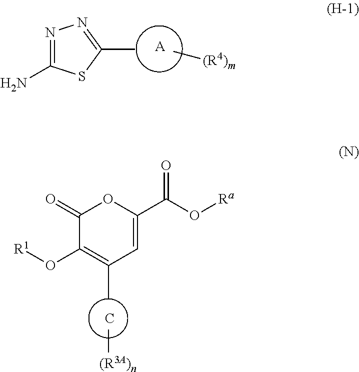
wherein R^{a} is hydrogen, C₁₋₆ alkyl, or C₁₋₆ haloalkyl, to provide a compound of Formula (I), or salt thereof.

31. The method of claim 30, wherein the compound of Formula (H-1), or salt thereof, is of Formula (H-2): 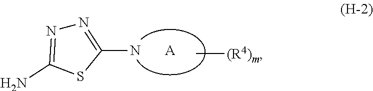
or salt thereof,
and wherein the method provides a compound of Formula (I′): 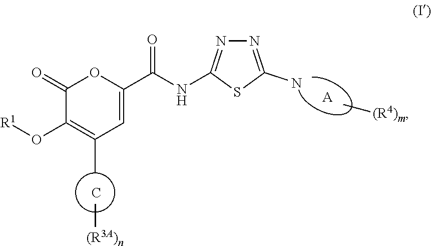
or salt thereof.

32. The method of claim 30 or 31, further comprising cross-coupling a compound of Formula (K-1), (K-2), or (K-3), or salt thereof, with a compound of Formula (D), or salt thereof, 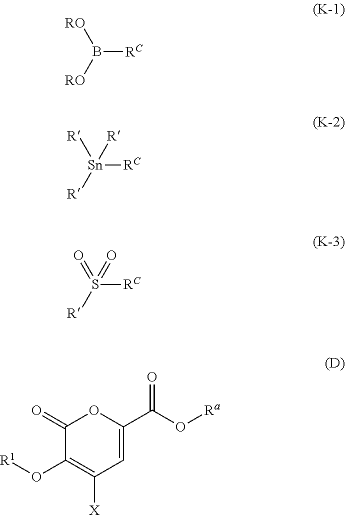
wherein:
R^{a} is C₁₋₆ alkyl or C₁₋₆ haloalkyl;
R^{C} is Ring C;
X is Cl, Br, or I;
each R is independently H, an optionally substituted C₁₋₆alkyl, or two R groups are joined to form a 5-6 membered ring; and
each R′ is independently an optionally substituted C₁₋₆ alkyl;
to provide a compound of Formula (N), or salt thereof.

33. A method of preparing a compound of Formula (I): 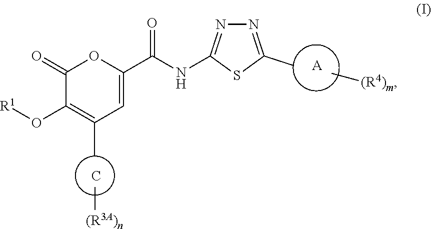
or a salt thereof, wherein Ring A, Ring C, R¹, R^{3A}, R⁴, m, and n are defined in claim 1, the method comprising cross-coupling a compound of Formula (K-1), (K-2), or (K-3), or salt thereof, with a compound of Formula (J-1), or salt thereof: 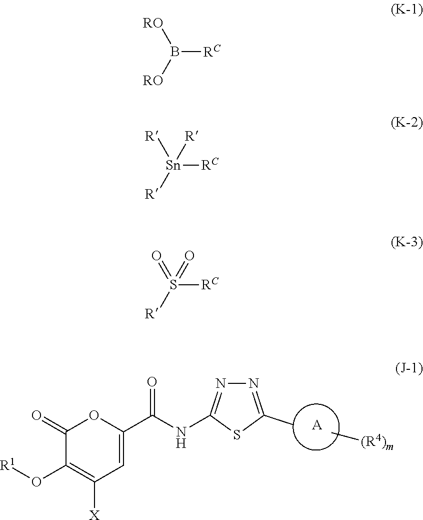
wherein:
R^{C} is Ring C;
X is Cl, Br, or I;
each R is independently H, an optionally substituted C₁₋₆alkyl, or two R groups are joined to form a 5-6 membered ring; and
each R′ is independently an optionally substituted C₁₋₆ alkyl;
to provide a compound of Formula (I), or salt thereof.

34. The method of claim 33, wherein the compound of Formula (J-1), or salt thereof, is of Formula (J-2): 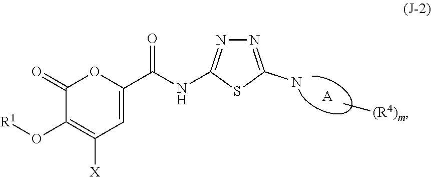
or salt thereof,
and wherein the method provides a compound of Formula (I′): 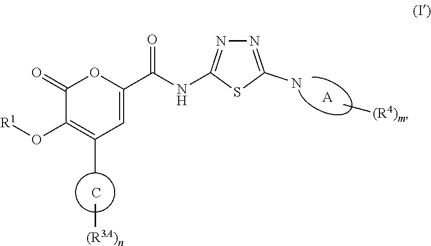
or salt thereof.

35. The method of claim 33, further comprising peptide coupling of a compound of Formula (H-1), or salt thereof, with a compound of Formula (D), or salt thereof: 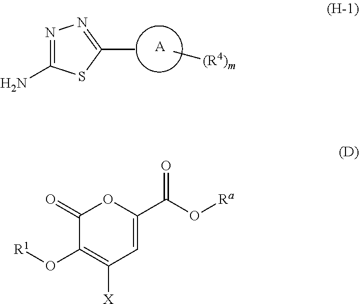
to provide a compound of Formula (J-1), or salt thereof.

36. The method of claim 34, further comprising peptide coupling of a compound of Formula (H-2), or salt thereof, with a compound of Formula (D), or salt thereof: 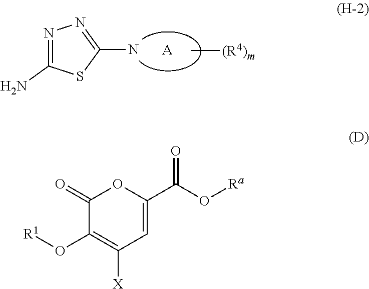
to provide a compound of Formula (J-2), or salt thereof.

37. The method of claim 30 or 35, further comprising reacting a hydrazine carbothioamide of Formula (G), or salt thereof, with a carboxylic acid containing compound of Formula (F-1), or salt thereof, or nitrile containing compound of Formula (F-2), or salt thereof: 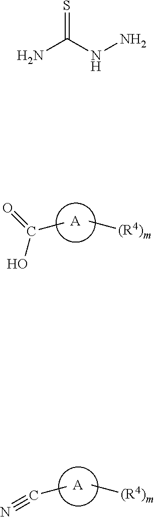
to provide a compound of Formula (H-1), or salt thereof.

38. The method of claim 31 or 36, further comprising coupling a compound Formula (M), or salt thereof, wherein Y is Cl, Br, or I, with an amine of Formula (L), or salt thereof: 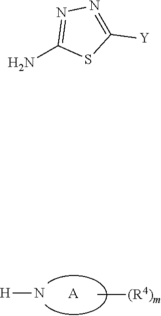
to provide a compound of Formula (H-2), or salt thereof.

39. The method of any one of claims 32, 35, and 36, further comprising:
(a) Protecting a compound of Formula (A), or salt thereof, to provide an alkyl ester of Formula (B): 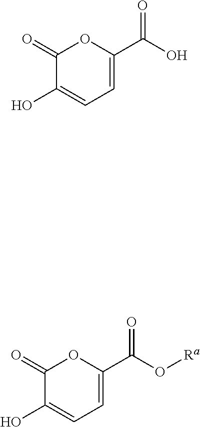
or salt thereof, wherein R^{a} is C₁₋₆ alkyl or C₁₋₆ haloalkyl;
(b) Halogenating the compound of Formula (B), or salt thereof, to provide a compound of Formula (C): 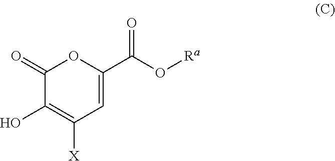
or salt thereof, wherein X is Cl, Br, or I;
(c) Protecting the hydroxyl group of the compound of Formula (C), or salt thereof, to provide a compound of Formula (D): 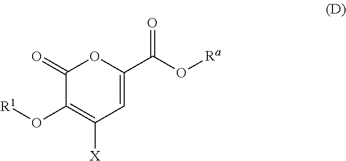
or salt thereof, wherein R¹ is as defined in claim 1; and
(d) optionally, deprotecting the compound of Formula (D), or salt thereof, to provide a carboxylic acid of Formula (D), wherein R^{a} is hydrogen.
